SEQUENCE LISTING (554 sequences)

SEQ ID NO: 1 - protein, 539 aa, synthetic construct
MSWKVVIIFSLLITPQHGLKESYLEESCSTITEGYLSVLRTGWYTNVFTLEVGDVENLTCADGPSLIKTELDLTKSALRELKTVSADQLAREEQIENPRQSRFVLGAIALGVATAAAVTAGVAIAKTIRLESEVTAIKNALKKTNEAVSTLGNGVRVLATAVRELKDFVSKNLTRAINKNKCDIDDLKMAVSFSQFNRRFLNVVRQFSDNAGITPAISLDLMTDAELARAVSNMPTSAGQIKLMLENRAMVRRKGFGILIGVYGSSVIYMVQLPIFGVIDTPCWIVKAAPSCSEKKGNYACLLREDQGWYCQNAGSTVYYPNEKDCETRGDHVFCDTAAGINVAEQSKECNINISTTNYPCKVSTGRHPISMVALSPLGALVACYKGVSCSIGSNRVGIIKQLNKGCSYITNQDADTVTIDNTVYQLSKVEGEQHVIKGRPVSSSFDPVKFPEDQFNVALDQVFENIENSQALVDQSNRILSSAEKGNTGFIIVIILIAVLGSSMILVSIFIIIKKTKKPTGAPPELSGVTNNGFIPHS

SEQ ID NO: 2 - protein, 539 aa, synthetic construct
MSWKVVIIFSLLITPQHGLKESYLEESCSTITEGYLSVLRTGWYTNVFTLEVGDVENLTCADGPSLIKTELDLTKSALRELRTVSADQLAREEQIENPRQSRFVLGAIALGVATAAAVTAGVAIAKTIRLESEVTAIKNALKKTNEAVSTLGNGVRVLATAVRELKDFVSKNLTRAINKNKCDIADLKMAVSFSQFNRRFLNVVRQFSDNAGITPAISLDLMTDAELARAVSNMPTSAGQIKLMLENRAMVRRKGFGILIGVYGSSVIYMVQLPIFGVIDTPCWIVKAAPSCSEKKGNYACLLREDQGWYCQNAGSTVYYPNEKDCETRGDHVFCDTAAGINVAEQSKECNINISTTNYPCKVSTGRHPISMVALSPLGALVACYKGVSCSIGSNRVGIIKQLNKGCSYITNQDADTVTIDNTVYQLSKVEGEQHVIKGRPVSSSFDPVKFPEDQFNVALDQVFESIENSQALVDQSNRILSSAEKGNTGFIIVIILIAVLGSTMILVSVFIIIKKTKKPTGAPPELSGVTNNGFIPHN

SEQ ID NO: 3 - protein, 539 aa, synthetic construct
MSWKVVIIFSLLITPQHGLKESYLEESCSTITEGYLSVLRTGWYTNVFTLEVGDVENLTCSDGPSLIKTELDLTKSALRELKTVSADQLAREEQIENPRQSRFVLGAIALGVATAAAVTAGVAIAKTIRLESEVTAIKNALKTTNEAVSTLGNGVRVLATAVRELKDFVSKNLTRAINKNKCDIDDLKMAVSFSQFNRRFLNVVRQFSDNAGITPAISLDLMTDAELARAVSNMPTSAGQIKLMLENRAMVRRKGFGILIGVYGSSVIYMVQLPIFGVIDTPCWIVKAAPSCSEKKGNYACLLREDQGWYCQNAGSTVYYPNEKDCETRGDHVFCDTAAGINVAEQSKECNINISTTNYPCKVSTGRHPISMVALSPLGALVACYKGVSCSIGSNRVGIIKQLNKGCSYITNQDADTVTIDNTVYQLSKVEGEQHVIKGRPVSSSFDPIKFPEDQFNVALDQVFENIENSQALVDQSNRILSSAEKGNTGFIIVIILIAVLGSSMILVSIFIIIKKTKKPTGAPPELSGVTNNGFIPHS

SEQ ID NO: 4 - protein, 539 aa, synthetic construct
MSWKVMIIISLLITPQHGLKESYLEESCSTITEGYLSVLRTGWYTNVFTLEVGDVENLTCTDGPSLIKTELDLTKSALRELKTVSADQLAREEQIENPRQSRFVLGAIALGVATAAAVTAGIAIAKTIRLESEVNAIKGALKTTNEAVSTLGNGVRVLATAVRELKEFVSKNLTSAINKNKCDIADLKMAVSFSQFNRRFLNVVRQFSDNAGITPAISLDLMNDAELARAVSYMPTSAGQIKLMLENRAMVRRKGFGILIGVYGSSVIYMVQLPIFGVINTPCWIIKAAPSCSEKDGNYACLLREDQGWYCKNAGSTVYYPNEKDCETRGDHVFCDTAAGINVAEQSRECNINISTTNYPCKVSTGRHPISMVALSPLGALVACYKGVSCSIGSNQVGIIKQLPKGCSYITNQDADTVTIDNTVYQLSKVEGEQHVIKGRPVSSSFDPIRFPEDQFNVALDQVFESIENSQALVDQSNKILNSAEKGNTGFIIVIILIAVLGLTMISVSIIIIIKKTRKPTGAPPELNGVTNGGFIPHS

SEQ ID NO: 5 - protein, 539 aa, synthetic construct
MSWKVMIIISLLITPQHGLKESYLEESCSTITEGYLSVLRTGWYTNVFTLEVGDVENLTCTDGPSLIKTELDLTKSALRELKTVSADQLAREEQIENPRQSRFVLGAIALGVATAAAVTAGIAIAKTIRLESEVNAIKGALKQTNEAVSTLGNGVRVLATAVRELKEFVSKNLTSAINRNKCDIADLKMAVSFSQFNRRFLNVVRQFSDNAGITPAISLDLMTDAELARAVSYMPTSAGQIKLMLENRAMVRRKGFGILIGVYGSSVIYMVQLPIFGVIDTPCWIIKAAPSCSEKNGNYACLLREDQGWYCKNAGSTVYYPNEKDCETRGDHVFCDTAAGINVAEQSRECNINISTTNYPCKVSTGRHPISMVALSPLGALVACYKGVSCSIGSNRVGIIKQLPKGCSYITNQDADTVTIDNTVYQLSKVEGEQHVIKGRPVSSSFDPIRFPEDQFNVALDQVFESIENSQALVEQSNKILNSAEKGNTGFIIVIILVAVLGLTMISVSIIIIIKKTRKPTGAPPELNGVTNGGFIPHS

SEQ ID NO: 6 - protein, 539 aa, synthetic construct
MSWKVMIIISLLITPQHGLKESYLEESCSTITEGYLSVLRTGWYTNVFTLEVGDVENLTCTDGPSLIKTELDLTKSALRELKTVSADQLAREEQIENPRQSRFVLGAIALGVATAAAVTAGIAIAKTIRLESEVNAIKGALKTTNEAVSTLGNGVRVLATAVRELKEFVSKNLTSAINKNKCDIADLKMAVSFSQFNRRFLNVVRQFSDNAGITPAISLDLMNDAELARAVSYMPTSAGQIKLMLENRAMVRRKGFGILIGVYGSSVIYMVQLPIFGVINTPCWIIKAAPSCSEKDGNYACLLREDQGWYCKNAGSTVYYPNEKDCETRGDHVFCDTAAGINVAEQSRECNINISTTNYPCKVSTGRHPISMVALSPLGALVACYKGVSCSTGSNQVGIIKQLPKGCSYITNQDADTVTIDNTVYQLSKVEGEQHVIKGRPVSNSFDPIRFPEDQFNVALDQVFESIENSQALVDQSNKILNSAEKGNTGFIIVIILIAVLGLTMISVSIIIIIKKTRKPAGAPPELNGVTNGGFIPHS

SEQ ID NO: 7 - protein, 27 aa, synthetic construct
GYIPEAPRDGQAYVRKDGEWVLLSTFL

SEQ ID NO: 8 - protein, 4 aa, synthetic construct
GSGS

SEQ ID NO: 9 - protein, 4 aa, synthetic construct
GGGS

SEQ ID NO: 10 - protein, 4 aa, synthetic construct
SAIG

SEQ ID NO: 11 - protein, 387 aa, synthetic construct
FVLGAIALGVATAAAVTAGVAIAKTIRLESEVTAIKNCLKKTNEAVCTLGNGVRVLATAVRELKDFVSKNLTRAINKNKCDIDDLKMAVSFSQFNRRFLNVVRQFSDNAGITPAISLDLMTDAELARAVSNMPTSAAQIKLMLENRAMVRRKGFGILIGVYGSSVIYMVQLPIFGVIDTPCWIVKAAPSCSEKKGNYACLLREDQGWYCQNAGSTVYYPNEKDCETRGDHVFCDTAAGINVAEQSKECNINISTTNYPCKVSIGRHPISMVALSPLGALVACYKGVSCSIGSNRVGIIKQLNKGCSYICNQDADTVTIDNTVYQLSKVEGECHVIKGRPVSSSFDPVKFPEDQFNVPLDQVFENIENSQALVDQSNRILSSAEKGNT

SEQ ID NO: 12 - protein, 84 aa, synthetic construct
LKESYLEESCSTITEGYLSVLRTGWYTNVFILEVGDVENLTCADGPSLIKTELDLTKSALRELKTVSADQLAREEQIENPRQSR

SEQ ID NO: 13 - protein, 387 aa, synthetic construct
FVLGAIALGVATAAAVTAGVAIAKTIRLESEVTAIKNCLKKTNEAVCTLGNGVRVLATAVRELKDFVSKNLTRAINKNKCDIADLKMAVSFSQFNRRFLNVVRQFSDNAGITPAISLDLMTDAELARAVSNMPTSAAQIKLMLENRAMVRRKGFGILIGVYGSSVIYMVQLPIFGVIDTPCWIVKAAPSCSEKKGNYACLLREDQGWYCQNAGSTVYYPNEKDCETRGDHVFCDTAAGINVAEQSKECNINISTTNYPCKVSIGRHPISMVALSPLGALVACYKGVSCSIGSNRVGIIKQLNKGCSYICNQDADTVTIDNTVYQLSKVEGECHVIKGRPVSSSFDPVKFPEDQFNVPLDQVFESIENSQALVDQSNRILSSAEKGNT

SEQ ID NO: 14 - protein, 84 aa, synthetic construct
LKESYLEESCSTITEGYLSVLRTGWYTNVFILEVGDVENLTCADGPSLIKTELDLTKSALRELRTVSADQLAREEQIENPRQSR

SEQ ID NO: 15 - protein, 387 aa, synthetic construct
FVLGAIALGVATAAAVTAGVAIAKTIRLESEVTAIKNCLKTTNEAVCTLGNGVRVLATAVRELKDFVSKNLTRAINKNKCDIDDLKMAVSFSQFNRRFLNVVRQFSDNAGITPAISLDLMTDAELARAVSNMPTSAAQIKLMLENRAMVRRKGFGILIGVYGSSVIYMVQLPIFGVIDTPCWIVKAAPSCSEKKGNYACLLREDQGWYCQNAGSTVYYPNEKDCETRGDHVFCDTAAGINVAEQSKECNINISTTNYPCKVSIGRHPISMVALSPLGALVACYKGVSCSIGSNRVGIIKQLNKGCSYICNQDADTVTIDNTVYQLSKVEGECHVIKGRPVSSSFDPIKFPEDQFNVPLDQVFENIENSQALVDQSNRILSSAEKGNT

SEQ ID NO: 16 - protein, 84 aa, synthetic construct
LKESYLEESCSTITEGYLSVLRTGWYTNVFILEVGDVENLTCSDGPSLIKTELDLTKSALRELKTVSADQLAREEQIENPRQSR

SEQ ID NO: 17 - protein, 387 aa, synthetic construct
FVLGAIALGVATAAAVTAGIAIAKTIRLESEVNAIKGCLKTTNEAVCTLGNGVRVLATAVRELKEFVSKNLTSAINKNKCDIADLKMAVSFSQFNRRFLNVVRQFSDNAGITPAISLDLMNDAELARAVSYMPTSAAQIKLMLENRAMVRRKGFGILIGVYGSSVIYMVQLPIFGVINTPCWIIKAAPSCSEKDGNYACLLREDQGWYCKNAGSTVYYPNEKDCETRGDHVFCDTAAGINVAEQSRECNINISTTNYPCKVSIGRHPISMVALSPLGALVACYKGVSCSIGSNQVGIIKQLPKGCSYICNQDADTVTIDNTVYQLSKVEGECHVIKGRPVSSSFDPIRFPEDQFNVPLDQVFESIENSQALVDQSNKILNSAEKGNT

SEQ ID NO: 18 - protein, 84 aa, synthetic construct
LKESYLEESCSTITEGYLSVLRTGWYTNVFILEVGDVENLTCTDGPSLIKTELDLTKSALRELKTVSADQLAREEQIENPRQSR

SEQ ID NO: 19 - protein, 387 aa, synthetic construct
FVLGAIALGVATAAAVTAGIAIAKTIRLESEVNAIKGCLKQTNEAVCTLGNGVRVLATAVRELKEFVSKNLTSAINRNKCDIADLKMAVSFSQFNRRFLNVVRQFSDNAGITPAISLDLMTDAELARAVSYMPTSAAQIKLMLENRAMVRRKGFGILIGVYGSSVIYMVQLPIFGVIDTPCWIIKAAPSCSEKNGNYACLLREDQGWYCKNAGSTVYYPNEKDCETRGDHVFCDTAAGINVAEQSRECNINISTTNYPCKVSIGRHPISMVALSPLGALVACYKGVSCSIGSNRVGIIKQLPKGCSYICNQDADTVTIDNTVYQLSKVEGECHVIKGRPVSSSFDPIRFPEDQFNVPLDQVFESIENSQALVEQSNKILNSAEKGNT

SEQ ID NO: 20 - protein, 84 aa, synthetic construct; identical to SEQ ID NO: 18

SEQ ID NO: 21 - protein, 387 aa, synthetic construct
FVLGAIALGVATAAAVTAGIAIAKTIRLESEVNAIKGCLKTTNEAVCTLGNGVRVLATAVRELKEFVSKNLTSAINKNKCDIADLKMAVSFSQFNRRFLNVVRQFSDNAGITPAISLDLMNDAELARAVSYMPTSAAQIKLMLENRAMVRRKGFGILIGVYGSSVIYMVQLPIFGVINTPCWIIKAAPSCSEKDGNYACLLREDQGWYCKNAGSTVYYPNEKDCETRGDHVFCDTAAGINVAEQSRECNINISTTNYPCKVSIGRHPISMVALSPLGALVACYKGVSCSTGSNQVGIIKQLPKGCSYICNQDADTVTIDNTVYQLSKVEGECHVIKGRPVSNSFDPIRFPEDQFNVPLDQVFESIENSQALVDQSNKILNSAEKGNT

SEQ ID NO: 22 - protein, 84 aa, synthetic construct; identical to SEQ ID NO: 18

SEQ ID NO: 23 - protein, 387 aa, synthetic construct
FVLGAIALGVATAAAVTAGVAIAKTIRLESEVTAIKNALKKTNEAVSTLGNGVRVLATAVRELKDFVSKNLTRAINKNKCDIDDLKMAVSFSQFNRRFLNVVRQFSDNAGITPAISLDLMTDAELARAVSNMPTSAGQIKLMLENRAMVRRKGFGILIGVYGSSVIYMVQLPIFGVIDTPCWIVKAAPSCSEKKGNYACLLREDQGWYCQNAGSTVYYPNEKDCETRGDHVFCDTAAGINVAEQSKECNINISTTNYPCKVSTCRHPISMVALSPLGALVACYKGVSCSIGSNRVGIIKQLNKGCSYITNQDADTVTIDNTVYQLSKVEGEQHVIKGRPVSSSFDPVKFPECQFNVALDQVFENIENSQALVDQSNRILSSAEKGNT

SEQ ID NO: 24 - protein, 84 aa, synthetic construct
LKESYLEESCSTITEGYLSVLRTGWYTNVFTLEVGDVENLTCADGPSLIKTELDLTKSALRELKTVSADQLAREEQIENPRQSR

SEQ ID NO: 25 - protein, 387 aa, synthetic construct
FVLGAIALGVATAAAVTAGVAIAKTIRLESEVTAIKNALKKTNEAVSTLGNGVRVLATAVRELKDFVSKNLTRAINKNKCDIDDLKMAVSFSQFNRRFLNVVRQFSDNAGITPAISLDLMTDAELARAVSNMPTSAGQIKLMLENRAMVRRKGFGILIGVYGSSVIYMVQLPIFGVIDTPCWIVKAAPSCSEKKGNYACLLREDQGWYCQNAGSTVYYPNEKDCETRGDHVFCDTAAGINVAEQSKECNINISTTNYPCKVSTGRHPISMVALSPLGALVACYKGVSCSIGSNRVGIIKQLNKGCSYICNQDADTVTIDNTVYQLSKVEGECHVIKGRPVSSSFDPVKFPEDQFNVALDQVFENIENSQALVDQSNRILSSAEKGNT

SEQ ID NO: 26 - protein, 84 aa, synthetic construct; identical to SEQ ID NO: 24

SEQ ID NO: 27 - protein, 387 aa, synthetic construct
FVLGAIALGVATAAAVTAGVAIAKTIRLESEVTACKNALKKTNEAVSTLGNGVRVLCTAVRELKDFVSKNLTRAINKNKCDIDDLKMAVSFSQFNRRFLNVVRQFSDNAGITPAISLDLMTDAELARAVSNMPTSAGQIKLMLENRAMVRRKGFGILIGVYGSSVIYMVQLPIFGVIDTPCWIVKAAPSCSEKKGNYACLLREDQGWYCQNAGSTVYYPNEKDCETRGDHVFCDTAAGINVAEQSKECNINISTTNYPCKVSTGRHPISMVALSPLGALVACYKGVSCSIGSNRVGIIKQLNKGCSYITNQDADTVTIDNTVYQLSKVEGEQHVIKGRPVSSSFDPVKFPEDQFNVALDQVFENIENSQALVDQSNRILSSAEKGNT

SEQ ID NO: 28 - protein, 84 aa, synthetic construct; identical to SEQ ID NO: 24

SEQ ID NO: 29 - protein, 387 aa, synthetic construct
FVLGAIALGVATAAAVTAGVAIAKTIRLESEVTAIKNCLKKTNEAVCTLGNGVRVLATAVRELKDFVSKNLTRAINKNKCDIDDLKMAVSFSQFNRRFLNVVRQFSDNAGITPAISLDLMTDAELARAVSNMPTSAGQIKLMLENRAMVRRKGFGILIGVYGSSVIYMVQLPIFGVIDTPCWIVKAAPSCSEKKGNYACLLREDQGWYCQNAGSTVYYPNEKDCETRGDHVFCDTAAGINVAEQSKECNINISTTNYPCKVSTGRHPISMVALSPLGALVACYKGVSCSIGSNRVGIIKQLNKGCSYITNQDADTVTIDNTVYQLSKVEGEQHVIKGRPVSSSFDPVKFPEDQFNVALDQVFENIENSQALVDQSNRILSSAEKGNT

SEQ ID NO: 30 - protein, 84 aa, synthetic construct; identical to SEQ ID NO: 24

SEQ ID NO: 31 - protein, 387 aa, synthetic construct
FVLGAIALGVATAAAVTAGVAIAKTIRLESEVTAIKNACKKTNEAVSTLGNGVRVLCTAVRELKDFVSKNLTRAINKNKCDIDDLKMAVSFSQFNRRFLNVVRQFSDNAGITPAISLDLMTDAELARAVSNMPTSAGQIKLMLENRAMVRRKGFGILIGVYGSSVIYMVQLPIFGVIDTPCWIVKAAPSCSEKKGNYACLLREDQGWYCQNAGSTVYYPNEKDCETRGDHVFCDTAAGINVAEQSKECNINISTTNYPCKVSTGRHPISMVALSPLGALVACYKGVSCSIGSNRVGIIKQLNKGCSYITNQDADTVTIDNTVYQLSKVEGEQHVIKGRPVSSSFDPVKFPEDQFNVALDQVFENIENSQALVDQSNRILSSAEKGNT

SEQ ID NO: 32 - protein, 84 aa, synthetic construct; identical to SEQ ID NO: 24

SEQ ID NO: 33 - protein, 387 aa, synthetic construct
FVLGAIALGVATAAAVTAGVAIAKTIRLESEVTAIKNACKKTNEAVSTLGNGVRVLATCVRELKDFVSKNLTRAINKNKCDIDDLKMAVSFSQFNRRFLNVVRQFSDNAGITPAISLDLMTDAELARAVSNMPTSAGQIKLMLENRAMVRRKGFGILIGVYGSSVIYMVQLPIFGVIDTPCWIVKAAPSCSEKKGNYACLLREDQGWYCQNAGSTVYYPNEKDCETRGDHVFCDTAAGINVAEQSKECNINISTTNYPCKVSTGRHPISMVALSPLGALVACYKGVSCSIGSNRVGIIKQLNKGCSYITNQDADTVTIDNTVYQLSKVEGEQHVIKGRPVSSSFDPVKFPEDQFNVALDQVFENIENSQALVDQSNRILSSAEKGNT

SEQ ID NO: 34 - protein, 84 aa, synthetic construct; identical to SEQ ID NO: 24

SEQ ID NO: 35 - protein, 387 aa, synthetic construct
FVLGAIALGVATAAAVTAGVAIAKTIRLESEVTAIKNALKKTNCAVSTLGNGVRVLACAVRELKDFVSKNLTRAINKNKCDIDDLKMAVSFSQFNRRFLNVVRQFSDNAGITPAISLDLMTDAELARAVSNMPTSAGQIKLMLENRAMVRRKGFGILIGVYGSSVIYMVQLPIFGVIDTPCWIVKAAPSCSEKKGNYACLLREDQGWYCQNAGSTVYYPNEKDCETRGDHVFCDTAAGINVAEQSKECNINISTTNYPCKVSTGRHPISMVALSPLGALVACYKGVSCSIGSNRVGIIKQLNKGCSYITNQDADTVTIDNTVYQLSKVEGEQHVIKGRPVSSSFDPVKFPEDQFNVALDQVFENIENSQALVDQSNRILSSAEKGNT

SEQ ID NO: 36 - protein, 84 aa, synthetic construct; identical to SEQ ID NO: 24

SEQ ID NO: 37 - protein, 387 aa, synthetic construct
FVLGAIALGVATAAAVTAGVAIAKTIRLESEVTAIKNALKKTNEACSTLGNGVRVCATAVRELKDFVSKNLTRAINKNKCDIDDLKMAVSFSQFNRRFLNVVRQFSDNAGITPAISLDLMTDAELARAVSNMPTSAGQIKLMLENRAMVRRKGFGILIGVYGSSVIYMVQLPIFGVIDTPCWIVKAAPSCSEKKGNYACLLREDQGWYCQNAGSTVYYPNEKDCETRGDHVFCDTAAGINVAEQSKECNINISTTNYPCKVSTGRHPISMVALSPLGALVACYKGVSCSIGSNRVGIIKQLNKGCSYITNQDADTVTIDNTVYQLSKVEGEQHVIKGRPVSSSFDPVKFPEDQFNVALDQVFENIENSQALVDQSNRILSSAEKGNT

SEQ ID NO: 38 - protein, 84 aa, synthetic construct; identical to SEQ ID NO: 24

SEQ ID NO: 39 - protein, 387 aa, synthetic construct
FVLGAIALGVATAAAVTAGVAIAKTIRLESEVTAIKNCLKKTNEAVCTLGNGVRVLATAVRELKDFVSKNLTRAINKNKCDIDDLKMAVSFSQFNRRFLNVVRQFSDNAGITPAISLDLMTDAELARAVSNMPTSAGQIKLMLENRAMVRRKGFGILIGVYGSSVIYMVQLPIFGVIDTPCWIVKAAPSCSEKKGNYACLLREDQGWYCQNAGSTVYYPNEKDCETRGDHVFCDTAAGINVAEQSKECNINISTTNYPCKVSTGRHPISMVALSPLGALVACYKGVSCSIGSNRVGIIKQLNKGCSYICNQDADTVTIDNTVYQLSKVEGECHVIKGRPVSSSFDPVKFPEDQFNVALDQVFENIENSQALVDQSNRILSSAEKGNT

SEQ ID NO: 40 - protein, 84 aa, synthetic construct; identical to SEQ ID NO: 24

SEQ ID NO: 41 - protein, 387 aa, synthetic construct
FVLGAIALGVATAAAVTAGVAIAKTIRLESEVTAIKNACKKTNEAVSTLGNGVRVLATCVRELKDFVSKNLTRAINKNKCDIDDLKMAVSFSQFNRRFLNVVRQFSDNAGITPAISLDLMTDAELARAVSNMPTSAGQIKLMLENRAMVRRKGFGILIGVYGSSVIYMVQLPIFGVIDTPCWIVKAAPSCSEKKGNYACLLREDQGWYCQNAGSTVYYPNEKDCETRGDHVFCDTAAGINVAEQSKECNINISTTNYPCKVSTGRHPISMVALSPLGALVACYKGVSCSIGSNRVGIIKQLNKGCSYICNQDADTVTIDNTVYQLSKVEGECHVIKGRPVSSSFDPVKFPEDQFNVALDQVFENIENSQALVDQSNRILSSAEKGNT

SEQ ID NO: 42 - protein, 84 aa, synthetic construct; identical to SEQ ID NO: 24

SEQ ID NO: 43 - protein, 387 aa, synthetic construct
FVLGAIALGVATAAAVTAGVAIAKTIRLESEVTAIKNALKKTNEAVSTLGNGVRVLATAVRELKDFVSKNLTRAINKNKCDIDDLKMAVSFSQFNRRFLNVVRQFSDNAGITPAISLDLMTDAELARAVSNMPTSAGQIKLMLENRAMVRRKGFGILIGVYGSSVIYMVQLPIFGVIDTPCWIVKAAPSCSEKKGNYACLLREDQGWYCQNAGSTVYYPNEKDCETRGDHVFCDTAAGINVAEQSKECNINISTTNYPCKVSTGRHPISMVALSPLGALVACYKGVSCSIGSNRVGIIKQLNKGCSYITNQDADTVTIDNTVYQLSKVEGEQHVIKGRPVSSSFDPVKFPEDQFNVALDQVFENIENSQALVDQSNRILSSAEKGNT

SEQ ID NO: 44 - protein, 84 aa, synthetic construct
LKESYLEESCSTITEGYLSVLRTGWYTNVFTLEVGDVENLTCADGPSPIKTELDLTKSALRELKTVSADQLAREEQIENPRQSR

SEQ ID NO: 45 - protein, 387 aa, synthetic construct
FVLGAIAPGVATAAAVTAGVAIAKTIRLESEVTAIKNALKKTNEAVSTLGNGVRVLATAVRELKDFVSKNLTRAINKNKCDIDDLKMAVSFSQFNRRFLNVVRQFSDNAGITPAISLDLMTDAELARAVSNMPTSAGQIKLMLENRAMVRRKGFGILIGVYGSSVIYMVQLPIFGVIDTPCWIVKAAPSCSEKKGNYACLLREDQGWYCQNAGSTVYYPNEKDCETRGDHVFCDTAAGINVAEQSKECNINISTTNYPCKVSTGRHPISMVALSPLGALVACYKGVSCSIGSNRVGIIKQLNKGCSYITNQDADTVTIDNTVYQLSKVEGEQHVIKGRPVSSSFDPVKFPEDQFNVALDQVFENIENSQALVDQSNRILSSAEKGNT

SEQ ID NO: 46 - protein, 84 aa, synthetic construct; identical to SEQ ID NO: 24

SEQ ID NO: 47 - protein, 387 aa, synthetic construct
FVLGAIALGVATAAAVTAGVAIAKTIRLEPEVTAIKNALKKTNEAVSTLGNGVRVLATAVRELKDFVSKNLTRAINKNKCDIDDLKMAVSFSQFNRRFLNVVRQFSDNAGITPAISLDLMTDAELARAVSNMPTSAGQIKLMLENRAMVRRKGFGILIGVYGSSVIYMVQLPIFGVIDTPCWIVKAAPSCSEKKGNYACLLREDQGWYCQNAGSTVYYPNEKDCETRGDHVFCDTAAGINVAEQSKECNINISTTNYPCKVSTGRHPISMVALSPLGALVACYKGVSCSIGSNRVGIIKQLNKGCSYITNQDADTVTIDNTVYQLSKVEGEQHVIKGRPVSSSFDPVKFPEDQFNVALDQVFENIENSQALVDQSNRILSSAEKGNT

SEQ ID NO: 48 - protein, 84 aa, synthetic construct; identical to SEQ ID NO: 24

SEQ ID NO: 49 - protein, 387 aa, synthetic construct
FVLGAIALGVATAAAVTAGVAIAKTIRLESEVTAIKNALKKTPEAVSTLGNGVRVLATAVRELKDFVSKNLTRAINKNKCDIDDLKMAVSFSQFNRRFLNVVRQFSDNAGITPAISLDLMTDAELARAVSNMPTSAGQIKLMLENRAMVRRKGFGILIGVYGSSVIYMVQLPIFGVIDTPCWIVKAAPSCSEKKGNYACLLREDQGWYCQNAGSTVYYPNEKDCETRGDHVFCDTAAGINVAEQSKECNINISTTNYPCKVSTGRHPISMVALSPLGALVACYKGVSCSIGSNRVGIIKQLNKGCSYITNQDADTVTIDNTVYQLSKVEGEQHVIKGRPVSSSFDPVKFPEDQFNVALDQVFENIENSQALVDQSNRILSSAEKGNT

SEQ ID NO: 50 - protein, 84 aa, synthetic construct; identical to SEQ ID NO: 24

SEQ ID NO: 51 - protein, 387 aa, synthetic construct
FVLGAIALGVATAAAVTAGVAIAKTIRLESEVTAIKNALKKTNEAVSTLGNGVRVLATAVRELKDFVSKNLTRAINKNKCDIDDPKMAVSFSQFNRRFLNVVRQFSDNAGITPAISLDLMTDAELARAVSNMPTSAGQIKLMLENRAMVRRKGFGILIGVYGSSVIYMVQLPIFGVIDTPCWIVKAAPSCSEKKGNYACLLREDQGWYCQNAGSTVYYPNEKDCETRGDHVFCDTAAGINVAEQSKECNINISTTNYPCKVSTGRHPISMVALSPLGALVACYKGVSCSIGSNRVGIIKQLNKGCSYITNQDADTVTIDNTVYQLSKVEGEQHVIKGRPVSSSFDPVKFPEDQFNVALDQVFENIENSQALVDQSNRILSSAEKGNT

SEQ ID NO: 52 - protein, 84 aa, synthetic construct; identical to SEQ ID NO: 24

SEQ ID NO: 53 - protein, 387 aa, synthetic construct
FVLGAIALGVATAAAVTAGVAIAKTIRLESEVTAIKNALKKTNEAVSTLGNGVRVLATAVRELKDFVSKNLTRAINKNKCDIDDLKMAVSFSQFNRRFLNVVRQFSDNAGITPAISLDLMTDAELARAVSNMPTSAGQIKLMLENRAMVRRKGFGILIGVYGSSVIYMVQLPIFGVIDTPCWIVKAAPSCSEKKGNYACLLREDQGWYCQNAGSTVYYPNEKDCETRGDHVFCDTAAGINVAEQSKECNINISTTNYPCKVSTGRHPISMVALSPLGALVACYKGVSCSIGSNRVGIIKQLNKGCSYITNQDADTVTIDNTVYQLSKVEGEQHVIKGRPVSSSFDPPKFPEDQFNVALDQVFENIENSQALVDQSNRILSSAEKGNT

SEQ ID NO: 54 - protein, 84 aa, synthetic construct; identical to SEQ ID NO: 24

SEQ ID NO: 55 - protein, 387 aa, synthetic construct
FVLGAIALGVATAAAVTAGVAIAKTIRLESEVTAIKNALKKTNEAVSTLGNGVRVLATAVRELKDFVSKNLTRAINKNKCDIDDLKMAVSFSQFNRRFLNVVRQFSDNAGITPAISLDLMTDAELARAVSNMPTSAGQIKLMLENRAMVRRKGFGILIGVYGSSVIYMVQLPIFGVIDTPCWIVKAAPSCSEKKGNYACLLREDQGWYCQNAGSTVYYPNEKDCETRGDHVFCDTAAGINVAEQSKECNINISTTNYPCKVSTGRHPISMVALSPLGALVACYKGVSCSIGSNRVGIIKQLNKGCSYITNQDADTVTIDNTVYQLSKVEGEQHVIKGRPVSSSFDPVKFPEDQFNVPLDQVFENIENSQALVDQSNRILSSAEKGNT

SEQ ID NO: 56 - protein, 84 aa, synthetic construct; identical to SEQ ID NO: 24

SEQ ID NO: 57 - protein, 387 aa, synthetic construct
FVLAAIALGVATAAAVTAGVAIAKTIRLESEVTAIKNALKKTNEAVSTLGNGVRVLATAVRELKDFVSKNLTRAINKNKCDIDDLKMAVSFSQFNRRFLNVVRQFSDNAGITPAISLDLMTDAELARAVSNMPTSAGQIKLMLENRAMVRRKGFGILIGVYGSSVIYMVQLPIFGVIDTPCWIVKAAPSCSEKKGNYACLLREDQGWYCQNAGSTVYYPNEKDCETRGDHVFCDTAAGINVAEQSKECNINISTTNYPCKVSTGRHPISMVALSPLGALVACYKGVSCSIGSNRVGIIKQLNKGCSYITNQDADTVTIDNTVYQLSKVEGEQHVIKGRPVSSSFDPVKFPEDQFNVALDQVFENIENSQALVDQSNRILSSAEKGNT

SEQ ID NO: 58 - protein, 84 aa, synthetic construct; identical to SEQ ID NO: 24

SEQ ID NO: 59 - protein, 387 aa, synthetic construct
FVLGAIALGVATAAAVTAAVAIAKTIRLESEVTAIKNALKKTNEAVSTLGNGVRVLATAVRELKDFVSKNLTRAINKNKCDIDDLKMAVSFSQFNRRFLNVVRQFSDNAGITPAISLDLMTDAELARAVSNMPTSAGQIKLMLENRAMVRRKGFGILIGVYGSSVIYMVQLPIFGVIDTPCWIVKAAPSCSEKKGNYACLLREDQGWYCQNAGSTVYYPNEKDCETRGDHVFCDTAAGINVAEQSKECNINISTTNYPCKVSTGRHPISMVALSPLGALVACYKGVSCSIGSNRVGIIKQLNKGCSYITNQDADTVTIDNTVYQLSKVEGEQHVIKGRPVSSSFDPVKFPEDQFNVALDQVFENIENSQALVDQSNRILSSAEKGNT

SEQ ID NO: 60 - protein, 84 aa, synthetic construct; identical to SEQ ID NO: 24

SEQ ID NO: 61 - protein, 386 aa, synthetic construct
FVLGAIALGVATAAAVTAGVAIAKTIRLESEVTAIKNALKKTNEAVSTLGNGVRVLATAVRELKDFVSKNLTRAINKNKCDIDDLKMAVSFSQFNRRFLNVVRQFSDNAGITPAISLDLMTDAELARAVSNMPTSAAQIKLMLENRAMVRRKGFGILIGVYGSSVIYMVQLPIFGVIDTPCWIVKAAPSCSEKKGNYACLLREDQGWYCQNAGSTVYYPNEKDCETRGDHVFCDTAAGINVAEQSKECNINISTTNYPCKVSTGRHPISMVALSPLGALVACYKGVSCSIGSNRVGIIKQLNKGCSYITNQDADTVTIDNTVYQLSKVEGEQHVIKGRPVSSSFDPVKFPEDQFNVALDQVFENIENSQALVDQSNRILSSAEKGN

SEQ ID NO: 62 - protein, 84 aa, synthetic construct; identical to SEQ ID NO: 24

SEQ ID NO: 63 - protein, 387 aa, synthetic construct; identical to SEQ ID NO: 43

SEQ ID NO: 64 - protein, 84 aa, synthetic construct; identical to SEQ ID NO: 12

SEQ ID NO: 65 - protein, 387 aa, synthetic construct
FVLGAIALGVATAAAVTAGVAIAKTIRLESEVTAIKNALKKTNEAVTTLGNGVRVLATAVRELKDFVSKNLTRAINKNKCDIDDLKMAVSFSQFNRRFLNVVRQFSDNAGITPAISLDLMTDAELARAVSNMPTSAGQIKLMLENRAMVRRKGFGILIGVYGSSVIYMVQLPIFGVIDTPCWIVKAAPSCSEKKGNYACLLREDQGWYCQNAGSTVYYPNEKDCETRGDHVFCDTAAGINVAEQSKECNINISTTNYPCKVSTGRHPISMVALSPLGALVACYKGVSCSIGSNRVGIIKQLNKGCSYITNQDADTVTIDNTVYQLSKVEGEQHVIKGRPVSSSFDPVKFPEDQFNVALDQVFENIENSQALVDQSNRILSSAEKGNT

SEQ ID NO: 66 - protein, 84 aa, synthetic construct; identical to SEQ ID NO: 24

SEQ ID NO: 67 - protein, 387 aa, synthetic construct
FVLGAIALGVATAAAVTAGVAIAKTIRLESEVTAIKNALKKTNEAVSTLGNGVRVLVTAVRELKDFVSKNLTRAINKNKCDIDDLKMAVSFSQFNRRFLNVVRQFSDNAGITPAISLDLMTDAELARAVSNMPTSAGQIKLMLENRAMVRRKGFGILIGVYGSSVIYMVQLPIFGVIDTPCWIVKAAPSCSEKKGNYACLLREDQGWYCQNAGSTVYYPNEKDCETRGDHVFCDTAAGINVAEQSKECNINISTTNYPCKVSTGRHPISMVALSPLGALVACYKGVSCSIGSNRVGIIKQLNKGCSYITNQDADTVTIDNTVYQLSKVEGEQHVIKGRPVSSSFDPVKFPEDQFNVALDQVFENIENSQALVDQSNRILSSAEKGNT

SEQ ID NO: 68 - protein, 84 aa, synthetic construct; identical to SEQ ID NO: 24

SEQ ID NO: 69 - protein, 387 aa, synthetic construct
FVLGAIALGVATAAAVTAGVAIAKTIRLESEVTAIKNALKKTNEAVSTLGNGVRVLATAVRELKDFVSKNLTRAINKNKCDIDDLKMAVSFSQFNRRFLNVVRQFSDNAGITPAISLDLMTDAELARAVSNMPTSAGQIKLMLENRAMVRRKGFGILIGVYGSSVIYMVQLPIFGVIDTPCWIVKAAPICSEKKGNYACLLREDQGWYCQNAGSTVYYPNEKDCETRGDHVFCDTAAGINVAEQSKECNINISTTNYPCKVSTGRHPISMVALSPLGALVACYKGVSCSIGSNRVGIIKQLNKGCSYITNQDADTVTIDNTVYQLSKVEGEQHVIKGRPVSSSFDPVKFPEDQFNVALDQVFENIENSQALVDQSNRILSSAEKGNT

SEQ ID NO: 70 - protein, 84 aa, synthetic construct; identical to SEQ ID NO: 24

SEQ ID NO: 71 - protein, 387 aa, synthetic construct
FVLGAIALGVATAAAVTAGVAIAKTIRLESEVTAIKNALKKTNEAVSTLGNGVRVLATAVRELKDFVSKNLTRAINKNKCDIDDLKMAVSFSQFNRRFLNVVRQFSDNAGITPAISLDLMTDAELARAVSNMPTSAGQIKLMLENRAMVRRKGFGILIGVYGSSVIYMVQLPIFGVIDTPCWIVKAAPSCSEKKGNYACLLREDQGWYCQNAGSTVYYPNEKDCETRGDHVFCDTAAGINVAEQSKECNINISTTNYPCKVSIGRHPISMVALSPLGALVACYKGVSCSIGSNRVGIIKQLNKGCSYITNQDADTVTIDNTVYQLSKVEGEQHVIKGRPVSSSFDPVKFPEDQFNVALDQVFENIENSQALVDQSNRILSSAEKGNT

SEQ ID NO: 72 - protein, 84 aa, synthetic construct; identical to SEQ ID NO: 24

SEQ ID NO: 73 - protein, 387 aa, synthetic construct
FVLGAIALGVATAAAVTAGVAIAKTIRLESEVTAIKNALKKTNEAVSTLGNGVRVLATAVRELKDFVSKNLTRAINKNKCDIDDLKMAVSFSQFNRRFLNVVRQFSDNAGITPAISLDLMTDAELARAVSNMPTSAGQIKLMLENRAMVRRKGFGILIGVYGSSVIYMVQLPIFGVIDTPCWIVKAAPSCSEKKGNYACLLREDQGWYCQNAGSTVYYPNEKDCETRGDHVFCDTAAGINVAEQSKECNINISTTNYPCKVSTGRHPISMVALSPLGALVACYKGVSCSIGSNRVGIIKQLNKGCSYITNQDADTVTIDNTVYQLSKVEGEQHVIKGRPVSSSFDPVKFPEDQFNVALDQVFENIENSQAFVDQSNRILSSAEKGNT

SEQ ID NO: 74 - protein, 84 aa, synthetic construct; identical to SEQ ID NO: 24

SEQ ID NO: 75 - protein, 387 aa, synthetic construct
FVLGAIALGVATAAAVTAGVAIAKTIRLESEVTAIKNCLKKTNEAVCTLGNGVRVLATAVRELKDFVSKNLTRAINKNKCDIDDLKMAVSFSQFNRRFLNVVRQFSDNAGITPAISLDLMTDAELARAVSNMPTSAGQIKLMLENRAMVRRKGFGILIGVYGSSVIYMVQLPIFGVIDTPCWIVKAAPSCSEKKGNYACLLREDQGWYCQNAGSTVYYPNEKDCETRGDHVFCDTAAGINVAEQSKECNINISTTNYPCKVSTGRHPISMVALSPLGALVACYKGVSCSIGSNRVGIIKQLNKGCSYICNQDADTVTIDNTVYQLSKVEGECHVIKGRPVSSSFDPVKFPEDQFNVPLDQVFENIENSQALVDQSNRILSSAEKGNT

SEQ ID NO: 76 - protein, 84 aa, synthetic construct; identical to SEQ ID NO: 24

SEQ ID NO: 77 - protein, 387 aa, synthetic construct
FVLGAIALGVATAAAVTAGVAIAKTIRLESEVTAIKNACKKTNEAVSTLGNGVRVLATCVRELKDFVSKNLTRAINKNKCDIDDLKMAVSFSQFNRRFLNVVRQFSDNAGITPAISLDLMTDAELARAVSNMPTSAGQIKLMLENRAMVRRKGFGILIGVYGSSVIYMVQLPIFGVIDTPCWIVKAAPSCSEKKGNYACLLREDQGWYCQNAGSTVYYPNEKDCETRGDHVFCDTAAGINVAEQSKECNINISTTNYPCKVSTGRHPISMVALSPLGALVACYKGVSCSIGSNRVGIIKQLNKGCSYICNQDADTVTIDNTVYQLSKVEGECHVIKGRPVSSSFDPVKFPEDQFNVPLDQVFENIENSQALVDQSNRILSSAEKGNT

SEQ ID NO: 78 - protein, 84 aa, synthetic construct; identical to SEQ ID NO: 24

SEQ ID NO: 79 - protein, 387 aa, synthetic construct
FVLGAIALGVATAAAVTAGVAIAKTIRLESEVTAIKNCLKKTNEAVCTLGNGVRVLATAVRELKDFVSKNLTRAINKNKCDIDDLKMAVSFSQFNRRFLNVVRQFSDNAGITPAISLDLMTDAELARAVSNMPTSAAQIKLMLENRAMVRRKGFGILIGVYGSSVIYMVQLPIFGVIDTPCWIVKAAPSCSEKKGNYACLLREDQGWYCQNAGSTVYYPNEKDCETRGDHVFCDTAAGINVAEQSKECNINISTTNYPCKVSTGRHPISMVALSPLGALVACYKGVSCSIGSNRVGIIKQLNKGCSYICNQDADTVTIDNTVYQLSKVEGECHVIKGRPVSSSFDPVKFPEDQFNVALDQVFENIENSQALVDQSNRILSSAEKGNT

SEQ ID NO: 80 - protein, 84 aa, synthetic construct; identical to SEQ ID NO: 24

SEQ ID NO: 81 - protein, 387 aa, synthetic construct
FVLGAIALGVATAAAVTAGVAIAKTIRLESEVTAIKNACKKTNEAVSTLGNGVRVLATCVRELKDFVSKNLTRAINKNKCDIDDLKMAVSFSQFNRRFLNVVRQFSDNAGITPAISLDLMTDAELARAVSNMPTSAAQIKLMLENRAMVRRKGFGILIGVYGSSVIYMVQLPIFGVIDTPCWIVKAAPSCSEKKGNYACLLREDQGWYCQNAGSTVYYPNEKDCETRGDHVFCDTAAGINVAEQSKECNINISTTNYPCKVSTGRHPISMVALSPLGALVACYKGVSCSIGSNRVGIIKQLNKGCSYICNQDADTVTIDNTVYQLSKVEGECHVIKGRPVSSSFDPVKFPEDQFNVALDQVFENIENSQALVDQSNRILSSAEKGNT

SEQ ID NO: 82 - protein, 84 aa, synthetic construct; identical to SEQ ID NO: 24

SEQ ID NO: 83 - protein, 387 aa, synthetic construct; identical to SEQ ID NO: 39

SEQ ID NO: 84 - protein, 84 aa, synthetic construct; identical to SEQ ID NO: 12

SEQ ID NO: 85 - protein, 387 aa, synthetic construct
FVLGAIALGVATAAAVTAGVAIAKTIRLESEVTAIKNCLKKTNEAVCTLGNGVRVLATAVRELKDFVSKNLTRAINKNKCDIDDLKMAVSFSQFNRRFLNVVRQFSDNAGITPAISLDLMTDAELARAVSNMPTSAGQIKLMLENRAMVRRKGFGILIGVYGSSVIYMVQLPIFGVIDTPCWIVKAAPSCSEKKGNYACLLREDQGWYCQNAGSTVYYPNEKDCETRGDHVFCDTAAGINVAEQSKECNINISTTNYPCKVSIGRHPISMVALSPLGALVACYKGVSCSIGSNRVGIIKQLNKGCSYICNQDADTVTIDNTVYQLSKVEGECHVIKGRPVSSSFDPVKFPEDQFNVALDQVFENIENSQALVDQSNRILSSAEKGNT

SEQ ID NO: 86 - protein, 84 aa, synthetic construct; identical to SEQ ID NO: 24

SEQ ID NO: 87 - protein, 387 aa, synthetic construct; identical to SEQ ID NO: 41

SEQ ID NO: 88 - protein, 84 aa, synthetic construct; identical to SEQ ID NO: 12

SEQ ID NO: 89 - protein, 387 aa, synthetic construct
FVLGAIALGVATAAAVTAGVAIAKTIRLESEVTAIKNACKKTNEAVSTLGNGVRVLATCVRELKDFVSKNLTRAINKNKCDIDDLKMAVSFSQFNRRFLNVVRQFSDNAGITPAISLDLMTDAELARAVSNMPTSAGQIKLMLENRAMVRRKGFGILIGVYGSSVIYMVQLPIFGVIDTPCWIVKAAPSCSEKKGNYACLLREDQGWYCQNAGSTVYYPNEKDCETRGDHVFCDTAAGINVAEQSKECNINISTTNYPCKVSIGRHPISMVALSPLGALVACYKGVSCSIGSNRVGIIKQLNKGCSYICNQDADTVTIDNTVYQLSKVEGECHVIKGRPVSSSFDPVKFPEDQFNVALDQVFENIENSQALVDQSNRILSSAEKGNT

SEQ ID NO: 90 - protein, 84 aa, synthetic construct; identical to SEQ ID NO: 24

SEQ ID NO: 91 - protein, 387 aa, synthetic construct
FVLGAIALGVATAAAVTAGVAIAKTIRLESEVTAIKNACKKTNEAVTTLGNGVRVLATCVRELKDFVSKNLTRAINKNKCDIDDLKMAVSFSQFNRRFLNVVRQFSDNAGITPAISLDLMTDAELARAVSNMPTSAGQIKLMLENRAMVRRKGFGILIGVYGSSVIYMVQLPIFGVIDTPCWIVKAAPSCSEKKGNYACLLREDQGWYCQNAGSTVYYPNEKDCETRGDHVFCDTAAGINVAEQSKECNINISTTNYPCKVSTGRHPISMVALSPLGALVACYKGVSCSIGSNRVGIIKQLNKGCSYICNQDADTVTIDNTVYQLSKVEGECHVIKGRPVSSSFDPVKFPEDQFNVALDQVFENIENSQALVDQSNRILSSAEKGNT

SEQ ID NO: 92 - protein, 84 aa, synthetic construct; identical to SEQ ID NO: 24

SEQ ID NO: 93 - protein, 387 aa, synthetic construct; identical to SEQ ID NO: 11

SEQ ID NO: 94 - protein, 84 aa, synthetic construct; identical to SEQ ID NO: 12

SEQ ID NO: 95 - protein, 387 aa, synthetic construct
FVLGAIALGVATAAAVTAGVAIAKTIRLESEVTAIKNACKKTNEAVTTLGNGVRVLATCVRELKDFVSKNLTRAINKNKCDIDDLKMAVSFSQFNRRFLNVVRQFSDNAGITPAISLDLMTDAELARAVSNMPTSAAQIKLMLENRAMVRRKGFGILIGVYGSSVIYMVQLPIFGVIDTPCWIVKAAPSCSEKKGNYACLLREDQGWYCQNAGSTVYYPNEKDCETRGDHVFCDTAAGINVAEQSKECNINISTTNYPCKVSIGRHPISMVALSPLGALVACYKGVSCSIGSNRVGIIKQLNKGCSYICNQDADTVTIDNTVYQLSKVEGECHVIKGRPVSSSFDPVKFPEDQFNVPLDQVFENIENSQALVDQSNRILSSAEKGNT

SEQ ID NO: 96 - protein, 84 aa, synthetic construct; identical to SEQ ID NO: 12

SEQ ID NO: 97 - protein, 387 aa, synthetic construct
FVLGAIALGVATAAAVTAGVAIAKTIRLESEVTAIKNALKKTNCAVTTLGNGVRVLACAVRELKDFVSKNLTRAINKNKCDIDDLKMAVSFSQFNRRFLNVVRQFSDNAGITPAISLDLMTDAELARAVSNMPTSAAQIKLMLENRAMVRRKGFGILIGVYGSSVIYMVQLPIFGVIDTPCWIVKAAPSCSEKKGNYACLLREDQGWYCQNAGSTVYYPNEKDCETRGDHVFCDTAAGINVAEQSKECNINISTTNYPCKVSIGRHPISMVALSPLGALVACYKGVSCSIGSNRVGIIKQLNKGCSYICNQDADTVTIDNTVYQLSKVEGECHVIKGRPVSSSFDPVKFPEDQFNVPLDQVFENIENSQALVDQSNRILSSAEKGNT

SEQ ID NO: 98 - protein, 84 aa, synthetic construct; identical to SEQ ID NO: 12

SEQ ID NO: 99 - protein, 539 aa, synthetic construct
MSWKVMIIISLLITPQHGLKESYLEESCSTITEGYLSVLRTGWYTNVFTLEVGDVENLTCTDGPSLIKTELDLTKSALRELKTVSADQLAREEQIENPRQSRFVLGAIALGVATAAAVTAGIAIAKTIRLESEVNAIKGALKTTNEAVSTLGNGVRVLATAVRELKEFVSKNLTSAINKNKCDIADLKMAVSFSQFNRRFLNVVRQFSDNAGITPAISLDLMNDAELARAVSYMPTSAGQIKLMLENRAMVRRKGFGILIGVYGSSVIYMVQLPIFGVINTPCWIIKAAPSCSEKDGNYACLLREDQGWYCKNAGSTVYYPNEKDCETRGDHVFCDTAAGINVAEQSRECNINISTTNYPCKVSTGRHPISMVALSPLGALVACYKGVSCSIGSNQVGIIKQLPKGCSYITNQDADTVTIDNTVYQLSKVEGEQHVIKGRPVSNSFDPIRFPEDQFNVALDQVFESIENSQALVDQSNKILNSAEKGNTGFIIVIILIAVLGLTMISVSIIIIIKKTRKPAGAPPELNGVTNGGFIPHS

SEQ ID NO: 100 - protein, 387 aa, synthetic construct
FVLGAIALGVATAAAVTAGIAIAKTIRLESEVNAIKGCLKTTNEAVCTLGNGVRVLATAVRELKEFVSKNLTSAINKNKCDIADLKMAVSFSQFNRRFLNVVRQFSDNAGITPAISLDLMNDAELARAVSYMPTSAAQIKLMLENRAMVRRKGFGILIGVYGSSVIYMVQLPIFGVINTPCWIIKAAPSCSEKDGNYACLLREDQGWYCKNAGSTVYYPNEKDCETRGDHVFCDTAAGINVAEQSRECNINISTTNYPCKVSIGRHPISMVALSPLGALVACYKGVSCSIGSNQVGIIKQLPKGCSYICNQDADTVTIDNTVYQLSKVEGECHVIKGRPVSNSFDPIRFPEDQFNVPLDQVFESIENSQALVDQSNKILNSAEKGNT

SEQ ID NO: 101 - protein, 84 aa, synthetic construct; identical to SEQ ID NO: 18

SEQ ID NO: 102 - DNA, 1617 bp, synthetic construct
  source 1..1617: mol_type = other DNA
atgagctggaaagtcatgatcatcatcagcctgctgatcacccctcagcacggcctgaaagagagctacctggaagaaagctgcagcaccatcaccgagggctacctgagcgtgctgagaaccggctggtacaccaacgtgttcaccctggaagtgggcgacgtggaaaacctgacctgcacagatggccccagcctgatcaagaccgagctggatctgacaaagagcgccctgcgcgagctgaaaaccgtgtctgcagatcagctggccagagaggaacagatcgagaaccccagacagagcagattcgtgctgggagctatcgccctgggagttgctacagctgctgctgtgacagccggaatcgccattgccaagaccatccggctggaaagcgaagtgaacgccatcaagggcgcactgaaaaccaccaacgaggccgtgtctaccctcggcaacggtgttagagtgctggccacagccgtgcgggaactgaaagaattcgtgtccaagaacctgaccagcgccatcaacaagaacaagtgcgacattgccgacctgaagatggccgtgtccttcagccagttcaaccggcggttcctgaatgtcgtgcggcagttctctgacaacgccggcatcacaccagccattagcctggacctgatgaacgacgccgaactggctagagccgtgtcttacatgcctacctctgccggccagatcaagctgatgctggaaaacagagccatggtccgacggaaaggcttcggcatcctgatcggcgtgtacggcagcagcgtgatctacatggtgcagctgcctatcttcggcgtgatcaacaccccttgctggatcatcaaggccgctcctagctgcagcgagaaggacggcaattacgcctgcctgctgagagaggaccaaggctggtactgcaagaatgccggcagcaccgtgtactaccccaacgagaaggattgcgagacacggggcgatcacgtgttctgtgatacagccgccggaatcaacgtggccgagcagagcagagagtgcaacatcaacatcagcaccacaaactacccctgcaaggtgtccatcggcagacaccctatcagcatggtggctctgtctccactgggagccctggtggcttgttataagggcgtgtcctgtagcatcggcagcaatcaagtgggcatcatcaagcagctgcccaagggctgctcctacatcaccaatcaggacgccgacaccgtgaccatcgacaataccgtgtatcagctgagcaaggtggaaggcgaacagcacgtgatcaagggcagacctgtgtccaacagcttcgaccccatcagattccccgaggaccagttcaatgtggccctggaccaggtgttcgagagcatcgagaatagccaggctctggtggaccagtccaacaagatcctgaactccgccgagaagggcaacaccggcttcatcatcgtgatcatcctgattgccgtgctgggcctgaccatgatcagcgtgtccatcatcattatcatcaagaaaacgcggaagcccgccggcgcccctccagaacttaatggcgtgaccaacggcggcttcattccccactct

SEQ ID NO: 103 - RNA, 1617 nt, synthetic construct
  source 1..1617: mol_type = other RNA
atgagctggaaagtcatgatcatcatcagcctgctgatcacccctcagcacggcctgaaagagagctacctggaagaaagctgcagcaccatcaccgagggctacctgagcgtgctgagaaccggctggtacaccaacgtgttcaccctggaagtgggcgacgtggaaaacctgacctgcacagatggccccagcctgatcaagaccgagctggatctgacaaagagcgccctgcgcgagctgaaaaccgtgtctgcagatcagctggccagagaggaacagatcgagaaccccagacagagcagattcgtgctgggagctatcgccctgggagttgctacagctgctgctgtgacagccggaatcgccattgccaagaccatccggctggaaagcgaagtgaacgccatcaagggcgcactgaaaaccaccaacgaggccgtgtctaccctcggcaacggtgttagagtgctggccacagccgtgcgggaactgaaagaattcgtgtccaagaacctgaccagcgccatcaacaagaacaagtgcgacattgccgacctgaagatggccgtgtccttcagccagttcaaccggcggttcctgaatgtcgtgcggcagttctctgacaacgccggcatcacaccagccattagcctggacctgatgaacgacgccgaactggctagagccgtgtcttacatgcctacctctgccggccagatcaagctgatgctggaaaacagagccatggtccgacggaaaggcttcggcatcctgatcggcgtgtacggcagcagcgtgatctacatggtgcagctgcctatcttcggcgtgatcaacaccccttgctggatcatcaaggccgctcctagctgcagcgagaaggacggcaattacgcctgcctgctgagagaggaccaaggctggtactgcaagaatgccggcagcaccgtgtactaccccaacgagaaggattgcgagacacggggcgatcacgtgttctgtgatacagccgccggaatcaacgtggccgagcagagcagagagtgcaacatcaacatcagcaccacaaactacccctgcaaggtgtccatcggcagacaccctatcagcatggtggctctgtctccactgggagccctggtggcttgttataagggcgtgtcctgtagcatcggcagcaatcaagtgggcatcatcaagcagctgcccaagggctgctcctacatcaccaatcaggacgccgacaccgtgaccatcgacaataccgtgtatcagctgagcaaggtggaaggcgaacagcacgtgatcaagggcagacctgtgtccaacagcttcgaccccatcagattccccgaggaccagttcaatgtggccctggaccaggtgttcgagagcatcgagaatagccaggctctggtggaccagtccaacaagatcctgaactccgccgagaagggcaacaccggcttcatcatcgtgatcatcctgattgccgtgctgggcctgaccatgatcagcgtgtccatcatcattatcatcaagaaaacgcggaagcccgccggcgcccctccagaacttaatggcgtgaccaacggcggcttcattccccactct

SEQ ID NO: 104 - DNA, 1617 bp, synthetic construct
  source 1..1617: mol_type = other DNA
atgagctggaaagtcatgatcatcatcagcctgctgatcacccctcagcacggcctgaaagagagctacctggaagaaagctgcagcaccatcaccgagggctacctgagcgtgctgagaaccggctggtacaccaacgtgttcatcctggaagtgggcgacgtggaaaacctgacctgcacagatggccccagcctgatcaagaccgagctggatctgacaaagagcgccctgcgcgagctgaaaaccgtgtctgcagatcagctggccagagaggaacagatcgagaaccccagacagagcagattcgtgctgggagctatcgccctgggagttgctacagctgctgctgtgacagccggaatcgccattgccaagaccatccggctggaaagcgaagtgaacgccatcaagggcgcactgaaaaccaccaacgaggccgtgtctaccctcggcaacggtgttagagtgctggccacagccgtgcgggaactgaaagaattcgtgtccaagaacctgaccagcgccatcaacaagaacaagtgcgacattgccgacctgaagatggccgtgtccttcagccagttcaaccggcggttcctgaatgtcgtgcggcagttctctgacaacgccggcatcacaccagccattagcctggacctgatgaacgacgccgaactggctagagccgtgtcttacatgcctacctctgccggccagatcaagctgatgctggaaaacagagccatggtccgacggaaaggcttcggcatcctgatcggcgtgtacggcagcagcgtgatctacatggtgcagctgcctatcttcggcgtgatcaacaccccttgctggatcatcaaggccgctcctagctgcagcgagaaggacggcaattacgcctgcctgctgagagaggaccaaggctggtactgcaagaatgccggcagcaccgtgtactaccccaacgagaaggattgcgagacacggggcgatcacgtgttctgtgatacagccgccggaatcaacgtggccgagcagagcagagagtgcaacatcaacatcagcaccacaaactacccctgcaaggtgtccatcggcagacaccctatcagcatggtggctctgtctccactgggagccctggtggcttgttataagggcgtgtcctgtagcatcggcagcaatcaagtgggcatcatcaagcagctgcccaagggctgctcctacatcaccaatcaggacgccgacaccgtgaccatcgacaataccgtgtatcagctgagcaaggtggaaggcgaacagcacgtgatcaagggcagacctgtgtccaacagcttcgaccccatcagattccccgaggaccagttcaatgtggccctggaccaggtgttcgagagcatcgagaatagccaggctctggtggaccagtccaacaagatcctgaactccgccgagaagggcaacaccggcttcatcatcgtgatcatcctgattgccgtgctgggcctgaccatgatcagcgtgtccatcatcattatcatcaagaaaacgcggaagcccgccggcgcccctccagaacttaatggcgtgaccaacggcggcttcattccccactct

SEQ ID NO: 105 - RNA, 1617 nt, synthetic construct
  source 1..1617: mol_type = other RNA
atgagctggaaagtcatgatcatcatcagcctgctgatcacccctcagcacggcctgaaagagagctacctggaagaaagctgcagcaccatcaccgagggctacctgagcgtgctgagaaccggctggtacaccaacgtgttcatcctggaagtgggcgacgtggaaaacctgacctgcacagatggccccagcctgatcaagaccgagctggatctgacaaagagcgccctgcgcgagctgaaaaccgtgtctgcagatcagctggccagagaggaacagatcgagaaccccagacagagcagattcgtgctgggagctatcgccctgggagttgctacagctgctgctgtgacagccggaatcgccattgccaagaccatccggctggaaagcgaagtgaacgccatcaagggcgcactgaaaaccaccaacgaggccgtgtctaccctcggcaacggtgttagagtgctggccacagccgtgcgggaactgaaagaattcgtgtccaagaacctgaccagcgccatcaacaagaacaagtgcgacattgccgacctgaagatggccgtgtccttcagccagttcaaccggcggttcctgaatgtcgtgcggcagttctctgacaacgccggcatcacaccagccattagcctggacctgatgaacgacgccgaactggctagagccgtgtcttacatgcctacctctgccggccagatcaagctgatgctggaaaacagagccatggtccgacggaaaggcttcggcatcctgatcggcgtgtacggcagcagcgtgatctacatggtgcagctgcctatcttcggcgtgatcaacaccccttgctggatcatcaaggccgctcctagctgcagcgagaaggacggcaattacgcctgcctgctgagagaggaccaaggctggtactgcaagaatgccggcagcaccgtgtactaccccaacgagaaggattgcgagacacggggcgatcacgtgttctgtgatacagccgccggaatcaacgtggccgagcagagcagagagtgcaacatcaacatcagcaccacaaactacccctgcaaggtgtccatcggcagacaccctatcagcatggtggctctgtctccactgggagccctggtggcttgttataagggcgtgtcctgtagcatcggcagcaatcaagtgggcatcatcaagcagctgcccaagggctgctcctacatcaccaatcaggacgccgacaccgtgaccatcgacaataccgtgtatcagctgagcaaggtggaaggcgaacagcacgtgatcaagggcagacctgtgtccaacagcttcgaccccatcagattccccgaggaccagttcaatgtggccctggaccaggtgttcgagagcatcgagaatagccaggctctggtggaccagtccaacaagatcctgaactccgccgagaagggcaacaccggcttcatcatcgtgatcatcctgattgccgtgctgggcctgaccatgatcagcgtgtccatcatcattatcatcaagaaaacgcggaagcccgccggcgcccctccagaacttaatggcgtgaccaacggcggcttcattccccactct

SEQ ID NO: 106-127 - skipped (22 entries)

SEQ ID NO: 128 - protein, 544 aa, synthetic construct
MSWKVVIIFSLLITPQHGLKESYLEESCSTITEGYLSVLRTGWYTNVFTLEVGDVENLTCADGPSLIKTELDLTKSALRELKTVSADQLAREEQIENPRQSRFVLGAIALGVATAAAVTAGVAIAKTIRLESEVTAIKNALKKTNEAVSTLGNGVRVLATAVRELKDFVSKNLTRAINKNKCDIDDLKMAVSFSQFNRRFLNVVRQFSDNAGITPAISLDLMTDAELARAVSNMPTSAGQIKLMLENRAMVRRKGFGILIGVYGSSVIYMVQLPIFGVIDTPCWIVKAAPSCSEKKGNYACLLREDQGWYCQNAGSTVYYPNEKDCETRGDHVFCDTAAGINVAEQSKECNINISTTNYPCKVSTGRHPISMVALSPLGALVACYKGVSCSIGSNRVGIIKQLNKGCSYITNQDADTVTIDNTVYQLSKVEGEQHVIKGRPVSSSFDPVKFPEDQFNVALDQVFENIENSQALVDQSNRILSSAEKGNTGGGSGYIPEAPRDGQAYVRKDGEWVLLSTFLGRSLEVLFQGPGSAWSHPQFEKAG

SEQ ID NO: 129 - protein, 544 aa, synthetic construct
MSWKVMIIISLLITPQHGLKESYLEESCSTITEGYLSVLRTGWYTNVFTLEVGDVENLTCTDGPSLIKTELDLTKSALRELKTVSADQLAREEQIENPRQSRFVLGAIALGVATAAAVTAGIAIAKTIRLESEVNAIKGALKTTNEAVSTLGNGVRVLATAVRELKEFVSKNLTSAINKNKCDIADLKMAVSFSQFNRRFLNVVRQFSDNAGITPAISLDLMNDAELARAVSYMPTSAGQIKLMLENRAMVRRKGFGILIGVYGSSVIYMVQLPIFGVINTPCWIIKAAPSCSEKDGNYACLLREDQGWYCKNAGSTVYYPNEKDCETRGDHVFCDTAAGINVAEQSRECNINISTTNYPCKVSTGRHPISMVALSPLGALVACYKGVSCSIGSNQVGIIKQLPKGCSYITNQDADTVTIDNTVYQLSKVEGEQHVIKGRPVSSSFDPIRFPEDQFNVALDQVFESIENSQALVDQSNKILNSAEKGNTGGGSGYIPEAPRDGQAYVRKDGEWVLLSTFLGRSLEVLFQGPGSAWSHPQFEKAG

SEQ ID NO: 130 - protein, 387 aa, synthetic construct
FVLGAIALGVATAAAVTAGIAIAKTIRLESEVNAIKGALKTTNEAVSTLGNGVRVLATAVRELKEFVSKNLTSAINKNKCDIADLKMAVSFSQFNRRFLNVVRQFSDNAGITPAISLDLMNDAELARAVSYMPTSAGQIKLMLENRAMVRRKGFGILIGVYGSSVIYMVQLPIFGVINTPCWIIKAAPSCSEKDGNYACLLREDQGWYCKNAGSTVYYPNEKDCETRGDHVFCDTAAGINVAEQSRECNINISTTNYPCKVSTGRHPISMVALSPLGALVACYKGVSCSIGSNQVGIIKQLPKGCSYICNQDADTVTIDNTVYQLSKVEGECHVIKGRPVSSSFDPIRFPEDQFNVALDQVFESIENSQALVDQSNKILNSAEKGNT

SEQ ID NO: 131 - protein, 84 aa, synthetic construct
LKESYLEESCSTITEGYLSVLRTGWYTNVFTLEVGDVENLTCTDGPSLIKTELDLTKSALRELKTVSADQLAREEQIENPRQSR

SEQ ID NO: 132 - protein, 387 aa, synthetic construct
FVLGAIALGVATAAAVTAGIAIAKTIRLESEVNAIKGCLKTTNEAVCTLGNGVRVLATAVRELKEFVSKNLTSAINKNKCDIADLKMAVSFSQFNRRFLNVVRQFSDNAGITPAISLDLMNDAELARAVSYMPTSAGQIKLMLENRAMVRRKGFGILIGVYGSSVIYMVQLPIFGVINTPCWIIKAAPSCSEKDGNYACLLREDQGWYCKNAGSTVYYPNEKDCETRGDHVFCDTAAGINVAEQSRECNINISTTNYPCKVSTGRHPISMVALSPLGALVACYKGVSCSIGSNQVGIIKQLPKGCSYITNQDADTVTIDNTVYQLSKVEGEQHVIKGRPVSSSFDPIRFPEDQFNVALDQVFESIENSQALVDQSNKILNSAEKGNT

SEQ ID NO: 133 - protein, 84 aa, synthetic construct; identical to SEQ ID NO: 131

SEQ ID NO: 134 - protein, 387 aa, synthetic construct
FVLGAIALGVATAAAVTAGIAIAKTIRLESEVNAIKGACKTTNEAVSTLGNGVRVLATCVRELKEFVSKNLTSAINKNKCDIADLKMAVSFSQFNRRFLNVVRQFSDNAGITPAISLDLMNDAELARAVSYMPTSAGQIKLMLENRAMVRRKGFGILIGVYGSSVIYMVQLPIFGVINTPCWIIKAAPSCSEKDGNYACLLREDQGWYCKNAGSTVYYPNEKDCETRGDHVFCDTAAGINVAEQSRECNINISTTNYPCKVSTGRHPISMVALSPLGALVACYKGVSCSIGSNQVGIIKQLPKGCSYITNQDADTVTIDNTVYQLSKVEGEQHVIKGRPVSSSFDPIRFPEDQFNVALDQVFESIENSQALVDQSNKILNSAEKGNT

SEQ ID NO: 135 - protein, 84 aa, synthetic construct; identical to SEQ ID NO: 131

SEQ ID NO: 136 - protein, 387 aa, synthetic construct
FVLGAIALGVATAAAVTAGIAIAKTIRLESEVNAIKGALKTTNCAVSTLGNGVRVLACAVRELKEFVSKNLTSAINKNKCDIADLKMAVSFSQFNRRFLNVVRQFSDNAGITPAISLDLMNDAELARAVSYMPTSAGQIKLMLENRAMVRRKGFGILIGVYGSSVIYMVQLPIFGVINTPCWIIKAAPSCSEKDGNYACLLREDQGWYCKNAGSTVYYPNEKDCETRGDHVFCDTAAGINVAEQSRECNINISTTNYPCKVSTGRHPISMVALSPLGALVACYKGVSCSIGSNQVGIIKQLPKGCSYITNQDADTVTIDNTVYQLSKVEGEQHVIKGRPVSSSFDPIRFPEDQFNVALDQVFESIENSQALVDQSNKILNSAEKGNT

SEQ ID NO: 137 - protein, 84 aa, synthetic construct; identical to SEQ ID NO: 131

SEQ ID NO: 138 - protein, 387 aa, synthetic construct
FVLGAIALGVATAAAVTAGIAIAKTIRLESEVNAIKGALKTTNEAVSCLGNGVCVLATAVRELKEFVSKNLTSAINKNKCDIADLKMAVSFSQFNRRFLNVVRQFSDNAGITPAISLDLMNDAELARAVSYMPTSAGQIKLMLENRAMVRRKGFGILIGVYGSSVIYMVQLPIFGVINTPCWIIKAAPSCSEKDGNYACLLREDQGWYCKNAGSTVYYPNEKDCETRGDHVFCDTAAGINVAEQSRECNINISTTNYPCKVSTGRHPISMVALSPLGALVACYKGVSCSIGSNQVGIIKQLPKGCSYITNQDADTVTIDNTVYQLSKVEGEQHVIKGRPVSSSFDPIRFPEDQFNVALDQVFESIENSQALVDQSNKILNSAEKGNT

SEQ ID NO: 139 - protein, 84 aa, synthetic construct; identical to SEQ ID NO: 131

SEQ ID NO: 140 - protein, 387 aa, synthetic construct
FVLGAIALGVATAAAVTAGIAIAKTIRLESEVNAIKGCLKTTNEAVCTLGNGVRVLATAVRELKEFVSKNLTSAINKNKCDIADLKMAVSFSQFNRRFLNVVRQFSDNAGITPAISLDLMNDAELARAVSYMPTSAGQIKLMLENRAMVRRKGFGILIGVYGSSVIYMVQLPIFGVINTPCWIIKAAPSCSEKDGNYACLLREDQGWYCKNAGSTVYYPNEKDCETRGDHVFCDTAAGINVAEQSRECNINISTTNYPCKVSTGRHPISMVALSPLGALVACYKGVSCSIGSNQVGIIKQLPKGCSYICNQDADTVTIDNTVYQLSKVEGECHVIKGRPVSSSFDPIRFPEDQFNVALDQVFESIENSQALVDQSNKILNSAEKGNT

SEQ ID NO: 141 - protein, 84 aa, synthetic construct; identical to SEQ ID NO: 131

SEQ ID NO: 142 - protein, 387 aa, synthetic construct
FVLGAIALGVATAAAVTAGIAIAKTIRLESEVNAIKGACKTTNEAVSTLGNGVRVLATCVRELKEFVSKNLTSAINKNKCDIADLKMAVSFSQFNRRFLNVVRQFSDNAGITPAISLDLMNDAELARAVSYMPTSAGQIKLMLENRAMVRRKGFGILIGVYGSSVIYMVQLPIFGVINTPCWIIKAAPSCSEKDGNYACLLREDQGWYCKNAGSTVYYPNEKDCETRGDHVFCDTAAGINVAEQSRECNINISTTNYPCKVSTGRHPISMVALSPLGALVACYKGVSCSIGSNQVGIIKQLPKGCSYICNQDADTVTIDNTVYQLSKVEGECHVIKGRPVSSSFDPIRFPEDQFNVALDQVFESIENSQALVDQSNKILNSAEKGNT

SEQ ID NO: 143 - protein, 84 aa, synthetic construct; identical to SEQ ID NO: 131

SEQ ID NO: 144-145 - skipped (2 entries)

SEQ ID NO: 146 - protein, 387 aa, synthetic construct
FVLGAIALGVATAAAVTAGIAIAKTIRLESEVNAIKGALKTTNEAVSTLGNGVRVLATAVRELKEFVSKNLTSAINKNKCDIADLKMAVSFSQFNRRFLNVVRQFSDNAGITPAISLDLMNDAELARAVSYMPTSAAQIKLMLENRAMVRRKGFGILIGVYGSSVIYMVQLPIFGVINTPCWIIKAAPSCSEKDGNYACLLREDQGWYCKNAGSTVYYPNEKDCETRGDHVFCDTAAGINVAEQSRECNINISTTNYPCKVSTGRHPISMVALSPLGALVACYKGVSCSIGSNQVGIIKQLPKGCSYITNQDADTVTIDNTVYQLSKVEGEQHVIKGRPVSSSFDPIRFPEDQFNVALDQVFESIENSQALVDQSNKILNSAEKGNT

SEQ ID NO: 147 - protein, 84 aa, synthetic construct; identical to SEQ ID NO: 131

SEQ ID NO: 148 - protein, 387 aa, synthetic construct
FVLGAIALGVATAAAVTAGIAIAKTIRLESEVNAIKGALKTTNEAVSTLGNGVRVLATAVRELKEFVSKNLTSAINKNKCDIADLKMAVSFSQFNRRFLNVVRQFSDNAGITPAISLDLMNDAELARAVSYMPTSAGQIKLMLENRAMVRRKGFGILIGVYGSSVIYMVQLPIFGVINTPCWIIKAAPSCSEKDGNYACLLREDQGWYCKNAGSTVYYPNEKDCETRGDHVFCDTAAGINVAEQSRECNINISTTNYPCKVSTGRHPISMVALSPLGALVACYKGVSCSIGSNQVGIIKQLPKGCSYITNQDADTVTIDNTVYQLSKVEGEQHVIKGRPVSSSFDPIRFPEDQFNVALDQVFESIENSQALVDQSNKILNSAEKGNT

SEQ ID NO: 149 - protein, 84 aa, synthetic construct; identical to SEQ ID NO: 18

SEQ ID NO: 150 - protein, 386 aa, synthetic construct
FVLGAIALGVATAAAVTAGIAIAKTIRLESEVNAIKGALKTTNEAVTTLGNGVRVLATAVRELKEFVSKNLTSAINKNKCDIADLKMAVSFSQFNRRFLNVVRQFSDNAGITPAISLDLMNDAELARAVSYMPTSAGQIKLMLENRAMVRRKGFGILIGVYGSSVIYMVQLPIFGVINTPCWIIKAAPSCSEKDGNYACLLREDQGWYCKNAGSTVYYPNEKDCETRGDHVFCDTAAGINVAEQSRECNINISTTNYPCKVSTGRHPISMVALSPLGALVACYKGVSCSIGSNQVGIIKQLPKGCSYITNQDADTVTIDNTVYQLSKVEGEQHVIKGRPVSSSFDPIRFPEDQFNVALDQVFESIENSQALVDQSNKILNSAEKGN

SEQ ID NO: 151 - protein, 84 aa, synthetic construct; identical to SEQ ID NO: 131

SEQ ID NO: 152 - protein, 387 aa, synthetic construct
FVLGAIALGVATAAAVTAGIAIAKTIRLESEVNAIKGALKTTNEAVSTLGNGVRVLATAVRELKEFVSKNLTSAINKNKCDIADLKMAVSFSQFNRRFLNVVRQFSDNAGITPAISLDLMNDAELARAVSYMPTSAGQIKLMLENRAMVRRKGFGILIGVYGSSVIYMVQLPIFGVINTPCWIIKAAPSCSEKDGNYACLLREDQGWYCKNAGSTVYYPNEKDCETRGDHVFCDTAAGINVAEQSRECNINISTTNYPCKVSIGRHPISMVALSPLGALVACYKGVSCSIGSNQVGIIKQLPKGCSYITNQDADTVTIDNTVYQLSKVEGEQHVIKGRPVSSSFDPIRFPEDQFNVALDQVFESIENSQALVDQSNKILNSAEKGNT

SEQ ID NO: 153 - protein, 84 aa, synthetic construct; identical to SEQ ID NO: 131

SEQ ID NO: 154 - protein, 387 aa, synthetic construct
FVLGAIALGVATAAAVTAGIAIAKTIRLESEVNAIKGALKTTNEAVSCLGNGVCVLATAVRELKEFVSKNLTSAINKNKCDIADLKMAVSFSQFNRRFLNVVRQFSDNAGITPAISLDLMNDAELARAVSYMPTSAGQIKLMLENRAMVRRKGFGILIGVYGSSVIYMVQLPIFGVINTPCWIIKAAPSCSEKDGNYACLLREDQGWYCKNAGSTVYYPNEKDCETRGDHVFCDTAAGINVAEQSRECNINISTTNYPCKVSTGRHPISMVALSPLGALVACYKGVSCSIGSNQVGIIKQLPKGCSYITNQDADTVTIDNTVYQLSKVEGEQHVIKGRPVSSSFDPIRFPEDQFNVPLDQVFESIENSQALVDQSNKILNSAEKGNT

SEQ ID NO: 155 - protein, 84 aa, synthetic construct; identical to SEQ ID NO: 131

SEQ ID NO: 156 - protein, 387 aa, synthetic construct
FVLGAIALGVATAAAVTAGIAIAKTIRLESEVNAIKGCLKTTNEAVCTLGNGVRVLATAVRELKEFVSKNLTSAINKNKCDIADLKMAVSFSQFNRRFLNVVRQFSDNAGITPAISLDLMNDAELARAVSYMPTSAGQIKLMLENRAMVRRKGFGILIGVYGSSVIYMVQLPIFGVINTPCWIIKAAPSCSEKDGNYACLLREDQGWYCKNAGSTVYYPNEKDCETRGDHVFCDTAAGINVAEQSRECNINISTTNYPCKVSTGRHPISMVALSPLGALVACYKGVSCSIGSNQVGIIKQLPKGCSYICNQDADTVTIDNTVYQLSKVEGECHVIKGRPVSSSFDPIRFPEDQFNVPLDQVFESIENSQALVDQSNKILNSAEKGNT

SEQ ID NO: 157 - protein, 84 aa, synthetic construct; identical to SEQ ID NO: 131

SEQ ID NO: 158 - protein, 387 aa, synthetic construct
FVLGAIALGVATAAAVTAGIAIAKTIRLESEVNAIKGACKTTNEAVSTLGNGVRVLATCVRELKEFVSKNLTSAINKNKCDIADLKMAVSFSQFNRRFLNVVRQFSDNAGITPAISLDLMNDAELARAVSYMPTSAGQIKLMLENRAMVRRKGFGILIGVYGSSVIYMVQLPIFGVINTPCWIIKAAPSCSEKDGNYACLLREDQGWYCKNAGSTVYYPNEKDCETRGDHVFCDTAAGINVAEQSRECNINISTTNYPCKVSTGRHPISMVALSPLGALVACYKGVSCSIGSNQVGIIKQLPKGCSYICNQDADTVTIDNTVYQLSKVEGECHVIKGRPVSSSFDPIRFPEDQFNVPLDQVFESIENSQALVDQSNKILNSAEKGNT

SEQ ID NO: 159 - protein, 84 aa, synthetic construct; identical to SEQ ID NO: 131

SEQ ID NO: 160 - protein, 387 aa, synthetic construct
FVLGAIALGVATAAAVTAGIAIAKTIRLESEVNAIKGCLKTTNEAVCTLGNGVRVLATAVRELKEFVSKNLTSAINKNKCDIADLKMAVSFSQFNRRFLNVVRQFSDNAGITPAISLDLMNDAELARAVSYMPTSAGQIKLMLENRAMVRRKGFGILIGVYGSSVIYMVQLPIFGVINTPCWIIKAAPSCSEKDGNYACLLREDQGWYCKNAGSTVYYPNEKDCETRGDHVFCDTAAGINVAEQSRECNINISTTNYPCKVSTGRHPISMVALSPLGALVACYKGVSCSIGSNQVGIIKQLPKGCSYITNQDADTVTIDNTVYQLSKVEGEQHVIKGRPVSSSFDPIRFPEDQFNVPLDQVFESIENSQALVDQSNKILNSAEKGNT

SEQ ID NO: 161 - protein, 84 aa, synthetic construct; identical to SEQ ID NO: 131

SEQ ID NO: 162 - protein, 387 aa, synthetic construct
FVLGAIALGVATAAAVTAGIAIAKTIRLESEVNAIKGCLKTTNEAVCTLGNGVRVLATAVRELKEFVSKNLTSAINKNKCDIADLKMAVSFSQFNRRFLNVVRQFSDNAGITPAISLDLMNDAELARAVSYMPTSAAQIKLMLENRAMVRRKGFGILIGVYGSSVIYMVQLPIFGVINTPCWIIKAAPSCSEKDGNYACLLREDQGWYCKNAGSTVYYPNEKDCETRGDHVFCDTAAGINVAEQSRECNINISTTNYPCKVSTGRHPISMVALSPLGALVACYKGVSCSIGSNQVGIIKQLPKGCSYICNQDADTVTIDNTVYQLSKVEGECHVIKGRPVSSSFDPIRFPEDQFNVALDQVFESIENSQALVDQSNKILNSAEKGNT

SEQ ID NO: 163 - protein, 84 aa, synthetic construct; identical to SEQ ID NO: 131

SEQ ID NO: 164 - protein, 387 aa, synthetic construct
FVLGAIALGVATAAAVTAGIAIAKTIRLESEVNAIKGACKTTNEAVSTLGNGVRVLATCVRELKEFVSKNLTSAINKNKCDIADLKMAVSFSQFNRRFLNVVRQFSDNAGITPAISLDLMNDAELARAVSYMPTSAAQIKLMLENRAMVRRKGFGILIGVYGSSVIYMVQLPIFGVINTPCWIIKAAPSCSEKDGNYACLLREDQGWYCKNAGSTVYYPNEKDCETRGDHVFCDTAAGINVAEQSRECNINISTTNYPCKVSTGRHPISMVALSPLGALVACYKGVSCSIGSNQVGIIKQLPKGCSYICNQDADTVTIDNTVYQLSKVEGECHVIKGRPVSSSFDPIRFPEDQFNVALDQVFESIENSQALVDQSNKILNSAEKGNT

SEQ ID NO: 165 - protein, 84 aa, synthetic construct; identical to SEQ ID NO: 131

SEQ ID NO: 166 - protein, 387 aa, synthetic construct; identical to SEQ ID NO: 138

SEQ ID NO: 167 - protein, 84 aa, synthetic construct; identical to SEQ ID NO: 18

SEQ ID NO: 168 - protein, 387 aa, synthetic construct; identical to SEQ ID NO: 132

SEQ ID NO: 169 - protein, 84 aa, synthetic construct; identical to SEQ ID NO: 18

SEQ ID NO: 170 - protein, 387 aa, synthetic construct; identical to SEQ ID NO: 142

SEQ ID NO: 171 - protein, 84 aa, synthetic construct; identical to SEQ ID NO: 18

SEQ ID NO: 172 - protein, 387 aa, synthetic construct
FVLGAIALGVATAAAVTAGIAIAKTIRLESEVNAIKGACKTTNEAVTTLGNGVRVLATCVRELKEFVSKNLTSAINKNKCDIADLKMAVSFSQFNRRFLNVVRQFSDNAGITPAISLDLMNDAELARAVSYMPTSAGQIKLMLENRAMVRRKGFGILIGVYGSSVIYMVQLPIFGVINTPCWIIKAAPSCSEKDGNYACLLREDQGWYCKNAGSTVYYPNEKDCETRGDHVFCDTAAGINVAEQSRECNINISTTNYPCKVSTGRHPISMVALSPLGALVACYKGVSCSIGSNQVGIIKQLPKGCSYITNQDADTVTIDNTVYQLSKVEGEQHVIKGRPVSSSFDPIRFPEDQFNVALDQVFESIENSQALVDQSNKILNSAEKGNT

SEQ ID NO: 173 - protein, 84 aa, synthetic construct; identical to SEQ ID NO: 131

SEQ ID NO: 174 - protein, 387 aa, synthetic construct; identical to SEQ ID NO: 172

SEQ ID NO: 175 - protein, 84 aa, synthetic construct; identical to SEQ ID NO: 18

SEQ ID NO: 176 - protein, 387 aa, synthetic construct
FVLGAIALGVATAAAVTAGIAIAKTIRLESEVNAIKGALKTTNEAVSCLGNGVCVLATAVRELKEFVSKNLTSAINKNKCDIADLKMAVSFSQFNRRFLNVVRQFSDNAGITPAISLDLMNDAELARAVSYMPTSAAQIKLMLENRAMVRRKGFGILIGVYGSSVIYMVQLPIFGVINTPCWIIKAAPSCSEKDGNYACLLREDQGWYCKNAGSTVYYPNEKDCETRGDHVFCDTAAGINVAEQSRECNINISTTNYPCKVSTGRHPISMVALSPLGALVACYKGVSCSIGSNQVGIIKQLPKGCSYITNQDADTVTIDNTVYQLSKVEGEQHVIKGRPVSSSFDPIRFPEDQFNVPLDQVFESIENSQALVDQSNKILNSAEKGNT

SEQ ID NO: 177 - protein, 84 aa, synthetic construct; identical to SEQ ID NO: 18

SEQ ID NO: 178 - protein, 387 aa, synthetic construct
FVLGAIALGVATAAAVTAGIAIAKTIRLESEVNAIKGCLKTTNEAVCTLGNGVRVLATAVRELKEFVSKNLTSAINKNKCDIADLKMAVSFSQFNRRFLNVVRQFSDNAGITPAISLDLMNDAELARAVSYMPTSAAQIKLMLENRAMVRRKGFGILIGVYGSSVIYMVQLPIFGVINTPCWIIKAAPSCSEKDGNYACLLREDQGWYCKNAGSTVYYPNEKDCETRGDHVFCDTAAGINVAEQSRECNINISTTNYPCKVSTGRHPISMVALSPLGALVACYKGVSCSIGSNQVGIIKQLPKGCSYICNQDADTVTIDNTVYQLSKVEGECHVIKGRPVSSSFDPIRFPEDQFNVPLDQVFESIENSQALVDQSNKILNSAEKGNT

SEQ ID NO: 179 - protein, 84 aa, synthetic construct; identical to SEQ ID NO: 18

SEQ ID NO: 180 - protein, 387 aa, synthetic construct; identical to SEQ ID NO: 17

SEQ ID NO: 181 - protein, 84 aa, synthetic construct; identical to SEQ ID NO: 18

SEQ ID NO: 182 - protein, 387 aa, synthetic construct
FVLGAIALGVATAAAVTAGIAIAKTIRLESEVNAIKGACKTTNEAVSTLGNGVRVLATCVRELKEFVSKNLTSAINKNKCDIADLKMAVSFSQFNRRFLNVVRQFSDNAGITPAISLDLMNDAELARAVSYMPTSAAQIKLMLENRAMVRRKGFGILIGVYGSSVIYMVQLPIFGVINTPCWIIKAAPSCSEKDGNYACLLREDQGWYCKNAGSTVYYPNEKDCETRGDHVFCDTAAGINVAEQSRECNINISTTNYPCKVSTGRHPISMVALSPLGALVACYKGVSCSIGSNQVGIIKQLPKGCSYICNQDADTVTIDNTVYQLSKVEGECHVIKGRPVSSSFDPIRFPEDQFNVPLDQVFESIENSQALVDQSNKILNSAEKGNT

SEQ ID NO: 183 - protein, 84 aa, synthetic construct; identical to SEQ ID NO: 18

SEQ ID NO: 184 - protein, 387 aa, synthetic construct
FVLGAIALGVATAAAVTAGIAIAKTIRLESEVNAIKGACKTTNEAVTTLGNGVRVLATCVRELKEFVSKNLTSAINKNKCDIADLKMAVSFSQFNRRFLNVVRQFSDNAGITPAISLDLMNDAELARAVSYMPTSAAQIKLMLENRAMVRRKGFGILIGVYGSSVIYMVQLPIFGVINTPCWIIKAAPSCSEKDGNYACLLREDQGWYCKNAGSTVYYPNEKDCETRGDHVFCDTAAGINVAEQSRECNINISTTNYPCKVSTGRHPISMVALSPLGALVACYKGVSCSIGSNQVGIIKQLPKGCSYICNQDADTVTIDNTVYQLSKVEGECHVIKGRPVSSSFDPIRFPEDQFNVPLDQVFESIENSQALVDQSNKILNSAEKGNT

SEQ ID NO: 185 - protein, 84 aa, synthetic construct; identical to SEQ ID NO: 131

SEQ ID NO: 186 - protein, 387 aa, synthetic construct; identical to SEQ ID NO: 184

SEQ ID NO: 187 - protein, 84 aa, synthetic construct; identical to SEQ ID NO: 18

SEQ ID NO: 188 - protein, 387 aa, synthetic construct
FVLGAIALGVATAAAVTAGIAIAKTIRLESEVNAIKGACKTTNEAVTTLGNGVRVLATCVRELKEFVSKNLTSAINKNKCDIADLKMAVSFSQFNRRFLNVVRQFSDNAGITPAISLDLMNDAELARAVSYMPTSAAQIKLMLENRAMVRRKGFGILIGVYGSSVIYMVQLPIFGVINTPCWIIKAAPSCSEKDGNYACLLREDQGWYCKNAGSTVYYPNEKDCETRGDHVFCDTAAGINVAEQSRECNINISTTNYPCKVSIGRHPISMVALSPLGALVACYKGVSCSIGSNQVGIIKQLPKGCSYICNQDADTVTIDNTVYQLSKVEGECHVIKGRPVSSSFDPIRFPEDQFNVPLDQVFESIENSQALVDQSNKILNSAEKGNT

SEQ ID NO: 189 - protein, 84 aa, synthetic construct; identical to SEQ ID NO: 18

SEQ ID NO: 190 - protein, 387 aa, synthetic construct; identical to SEQ ID NO: 154

SEQ ID NO: 191 - protein, 84 aa, synthetic construct; identical to SEQ ID NO: 18

SEQ ID NO: 192 - protein, 387 aa, synthetic construct; identical to SEQ ID NO: 160

SEQ ID NO: 193 - protein, 84 aa, synthetic construct; identical to SEQ ID NO: 18

SEQ ID NO: 194 - protein, 387 aa, synthetic construct; identical to SEQ ID NO: 158

SEQ ID NO: 195 - protein, 84 aa, synthetic construct; identical to SEQ ID NO: 18

SEQ ID NO: 196 - protein, 387 aa, synthetic construct
FVLGAIALGVATAAAVTAGIAIAKTIRLESEVNAIKGACKTTNEAVTTLGNGVRVLATCVRELKEFVSKNLTSAINKNKCDIADLKMAVSFSQFNRRFLNVVRQFSDNAGITPAISLDLMNDAELARAVSYMPTSAGQIKLMLENRAMVRRKGFGILIGVYGSSVIYMVQLPIFGVINTPCWIIKAAPSCSEKDGNYACLLREDQGWYCKNAGSTVYYPNEKDCETRGDHVFCDTAAGINVAEQSRECNINISTTNYPCKVSTGRHPISMVALSPLGALVACYKGVSCSIGSNQVGIIKQLPKGCSYITNQDADTVTIDNTVYQLSKVEGEQHVIKGRPVSSSFDPIRFPEDQFNVPLDQVFESIENSQALVDQSNKILNSAEKGNT

SEQ ID NO: 197 - protein, 84 aa, synthetic construct; identical to SEQ ID NO: 131

SEQ ID NO: 198 - protein, 387 aa, synthetic construct; identical to SEQ ID NO: 196

SEQ ID NO: 199 - protein, 84 aa, synthetic construct; identical to SEQ ID NO: 18

SEQ ID NO: 200 - protein, 387 aa, synthetic construct; identical to SEQ ID NO: 178

SEQ ID NO: 201 - protein, 84 aa, synthetic construct; identical to SEQ ID NO: 131

SEQ ID NO: 202 - protein, 387 aa, synthetic construct; identical to SEQ ID NO: 182

SEQ ID NO: 203 - protein, 84 aa, synthetic construct; identical to SEQ ID NO: 131

SEQ ID NO: 204 - protein, 387 aa, synthetic construct
FVLGAIALGVATAAAVTAGIAIAKTIRLESEVNAIKGALKTTNEAVTTLGNGVRVLATAVRELKEFVSKNLTSAINKNKCDIADLKMAVSFSQFNRRFLNVVRQFSDNAGITPAISLDLMNDAELARAVSYMPTSAGQIKLMLENRAMVRRKGFGILIGVYGSSVIYMVQLPIFGVINTPCWIIKAAPSCSEKDGNYACLLREDQGWYCKNAGSTVYYPNEKDCETRGDHVFCDTAAGINVAEQSRECNINISTTNYPCKVSTGRHPISMVALSPLGALVACYKGVSCSIGSNQVGIIKQLPKGCSYITNQDADTVTIDNTVYQLSKVEGEQHVIKGRPVSSSFDPIRFPEDQFNVPLDQVFESIENSQALVDQSNKILNSAEKGNT

SEQ ID NO: 205 - protein, 84 aa, synthetic construct; identical to SEQ ID NO: 18

SEQ ID NO: 206 - protein, 555 aa, synthetic construct
MQSSEILLLVYSSLLLSSSLCQIPVDKLSNVGVIINEGKLLKIAGSYESRYIVLSLVPSIDLQDGCGTTQIIQYKNLLNRLLIPLKDALDLQESLITITNDTTVTNDNPQTRFFGAVIGTIALGVATAAQITAGIALAEAREARKDIALIKDSIVKTHNSVEFIQRGIGEQIIALKTLQDFVNDEIRPAIGELRCETTALKLGIKLTQHYSELATAFSSNLGTIGEKSLTLQALSSLYSANITEILSTIKKDKSDIYDIIYTEQVKGTVIDVDLEKYMVTLLVKIPILSEIPGVLIYRASSISYNIEGEEWHVAIPNYIINKASSLGGADVTNCIESKLAYICPRDPTQLIPDNQQKCILGDVSKCPVTKVINNLVPKFAFINGGVVANCIASTCTCGTNRIPVNQDRSKGVTFLTYTNCGLIGINGIELYANKRGRDTTWGNQIIKVGPAVSIRPVDISLNLASATNFLEESKTELMKARAIISAVGGWHNKESTQIIIIIIVCILIIIICGILYYLYRVRRLLIMINSTNNSPINAYTLESRMKNPYMGNHSN

SEQ ID NO: 207 - protein, 555 aa, synthetic construct
MQSSEVFLLVYSSLLLSSSLCQIPIDKLSNVGVIINEGKLLKIAGSYESRYIVLSLVPSIDLQDGCGTTQIIQYKNLLNRLLIPLKDALDLQESLITITNDTTVTNDNPQTRFFGAVIGTIALGVATAAQITAGIALAEAREARKDIALIKDSIVKTHNSVEFIQRGIGEQIIALKTLQDFVNDEIRPAIGELRCETTALKLGIKLTQHYSELATAFSSNLGTIGEKSLTLQALSSLYSANITEILSTIKKDKSDIYDIIYTEQVKGTVIDVDLEKYMVTLLVKIPILSEIPGVLIYRASSISYNIEGEEWHVAIPSYIINKASSLGGADVTNCIESKLAYICPRDPTQLIPDNQQKCILGDVSKCPVTKVINNLVPKFAFINGGVVANCIASTCTCGTNRIPVNQDRSKGVTFLTYTNCGLIGINGIELYANKRGRDTTWGNQIIKVGPAVSIRPVDISLNLASATNFLEESKTELMRARAIISAVGGWHNKESTQIIIIIIVCVLIIIICGILYYLYRVRSLLIMINSTNNSPINAYTLESRMKNPYMGNHPN

SEQ ID NO: 208 - protein, 555 aa, synthetic construct
MQKSEILFLVYSSLLLSSSLCQIPVEKLSNVGVIINEGKLLKIAGSYESRYIVLSLVPSIDLQDGCGTTQIIQYKNLLNRLLIPLKDALDLQESLITITNDTTVTNDNPQTRFFGAVIGTIALGVATAAQITAGIALAEAREARKDIALIKDSIVKTHNSVELIQRGIGEQIIALKTLQDFVNDEIRPAIGELRCETTALKLGIKLTQHYSELATAFSSNLGTIGEKSLTLQALSSLYSANITEILSTTKKDKSDIYDIIYTEQVKGTVIDVDLEKYMVTLLVKIPILSEIPGVLIYRASSISYNIEGEEWHVAIPNYIINKASSLGGADVTNCIESKLAYICPRDPTQLIPDNQQKCILGDVSKCPVTKVINNLVPKFAFINGGVVANCIASTCTCGTNRIPVNQDRSRGVTFLTYTNCGLIGINGIELYANKRGRDTTWGNQIIKVGPAVSIRPVDISLNLASATNFLEESKTELMKARAIISAVGGWHNTESTQIIMIIIVCILIIIICGILYYLYRVRRLLVMINSTHNSPVNAYTLESRMRNPYMGNNSN

SEQ ID NO: 209 - protein, 555 aa, synthetic construct
MQSSEILILVYSSLLLSSSLCQIPVDKLSNVGVIINEGKLLKIAGSYESRYIVLSLVPSIDLQDGCGTTQIIQYKNLLNRLLIPLKDALDLQESLITITNDTTVTNDNPQTRFFGAVIGTIALGVATAAQITAGIALAEAREARKDIALIKDSIVKTHNSVEFIQRGIGEQIIALKTLQDFVNDEIRPAIGELRCETTALKLGIKLTQHYSELATAFSSNLGTIGEKSLTLQALSSLYSANITEILSTIKKDKSDIYDIIYTEQVKGTVIDVDLEKYMVTLLVKIPILSEIPGVLIYRASSISYNIEGEEWHVAIPNYIISKASSLGGADVTSCIESKLAYICPRDPTQLIPDNQQKCILGDVSKCPVTKVINNLVPKFAFINGGVVANCIASTCTCGTNRIPVNQDRSKGVTFLTYTNCGLIGINGIELYANKRGRDTTWGNQIIKVGPAVSIRPVDISLNLASATNFLEESKTELMKARAIISAVGGWHNTESTQIIIIIIVCILIIIICGILYYLYRVRRLLVMINSTNNSPINAYTLESRMRNPYMGNHSN

SEQ ID NO: 210 - protein, 555 aa, synthetic construct
MQSSEILILVYSSLLLSSSLCQIPVDKLSNVGVIINEGKLLKIAGSYESRYIVLSLVPSIDLQDGCGTTQIIQYKNLLNRLLIPLKDALDLQESLITITNDTTVTNDNPQTRFFGAVIGTIALGVATAAQITAGIALAEAREARKDIALIKDSIVKTHNSVEFIQRGIGEQIIALKTLQDFVNDEIRPAIGELRCETTALKLGIKLTQHYSELATAFSSNLGTIGEKSLTLQALSSLYSANITEILSTIKKDKSDIYDIIYTEQVKGTVIDVDLEKYMVTLLVKIPILSEIPGVLIYRASSISYNIEGEEWHVAIPNYIISKASSLGGADVTNCIESKLAYICPRDPTQLIPDNQQKCILGDVSKCPVTKVINNLVPKFAFINGGVVANCIASTCTCGTNRIPVNQDRSKGVTFLTYTNCGLIGINGIELYANKRGRDTTWGNQIIKVGPAVSIRPVDISLNLASATNFLEESKTELMKARAIISAVGGWHNTESTQIIIIIIVCILIIIICGILYYLYRIRRLLVMINSTNNSPINAYTLESRMRNPYMGNHSN

SEQ ID NO: 211 - protein, 532 aa, synthetic construct
MQSSEILLLVYSSLLLSSSLCQIPVDKLSNVGVIINEGKLLKIAGSYESRYIVLSLVPSIDLQDGCGTTQIIQYKNLLNRLLIPLKDALDLQESLITITNDTTVTNDNPQTRFFGAVIGTIALGVATAAQITAGIALAEAREARKDIALIKDSIVKTHNSVEFIQRGIGEQIIALKTLQDFVNDEIRPAIGELRCETTALKLGIKLTQHYSELATAFSSNLGTIGEKSLTLQALSSLYSANITEILSTIKKDKSDIYDIIYTEQVKGTVIDVDLEKYMVTLLVKIPILSEIPGVLIYRASSISYNIEGEEWHVAIPNYIINKASSLGGADVTNCIESKLAYICPRDPTQLIPDNQQKCILGDVSKCPVTKVINNLVPKFAFINGGVVANCIASTCTCGTNRIPVNQDRSKGVTFLTYTNCGLIGINGIELYANKRGRDTTWGNQIIKVGPAVSIRPVDISLNLASATNFLEESKTELGGGSGYIPEAPRDGQAYVRKDGEWVLLSTFLGRSLEVLFQGPGSAWSHPQFEKAG

SEQ ID NO: 212 - protein, 365 aa, synthetic construct
FFGAVIGTIALGVATAAQITACIALAEAREARKDIALIKDSIVKTHNSVEFIQRGIGEQIIALKTLQDFVNDEIRPAIGELRCETTALKLGIKLTQHYSELATAFSSNLGTIGEKSLTLQALSSLYSANITEILSTIKKDKSDIYDIIYTEQVKGTVIDVDLEKYMVTLLVKIPILSEIPGVLIYRASSISYNIEGEEWHVAIPNYIINKASSLGGADVTNCIESKLAYICPRDPTQLIPDNQQKCILGDVSKCPVTKVINNLVPKFAFINGGVVANCIASTCTCGTNRIPVNQDRSKGVTFLTYTNCGLIGINGIELYANKRGRDTTWGNQIIKVGPAVSIRPVDISLNLASATNFLEESKTEL

SEQ ID NO: 213 - protein, 91 aa, synthetic construct
QIPVDKLSNVGVIINEGKLLKIAGSYESRYIVLSLVPSIDLQDGCGTTQIIQYKNLLNRLLIPLKDALDLCESLITITNDTTVTNDNPQTR

SEQ ID NO: 214 - protein, 365 aa, synthetic construct
FFGAVIGTIALGVATPAQITAGIALAEAREARKDIALIKDSIVKTHNSVEFIQRGIGEQIIALKTLQDFVNDEIRPAIGELRCETTALKLGIKLTQHYSELATAFSSNLGTIGEKSLTLQALSSLYSANITEILSTIKKDKSDIYDIIYTEQVKGTVIDVDLEKYMVTLLVKIPILSEIPGVLIYRASSISYNIEGEEWHVAIPNYIINKASSLGGADVTNCIESKLAYICPRDPTQLIPDNQQKCILGDVSKCPVTKVINNLVPKFAFINGGVVANCIASTCTCGTNRIPVNQDRSKGVTFLTYTNCGLIGINGIELYANKRGRDTTWGNQIIKVGPAVSIRPVDISLNLASATNFLEESKTEL

SEQ ID NO: 215 - protein, 91 aa, synthetic construct
QIPVDKLSNVGVIINEGKLLKIAGSYESRYIVLSLVPSIDLQDGCGTTQIIQYKNLLNRLLIPLKDALDLQESLITITNDTTVTNDNPQTR

SEQ ID NO: 216 - protein, 365 aa, synthetic construct
FFGAVIGTIALGVATAAQITAGIALAEAREARKDIALIKDSIVKTHNSVEFIQRGIGEQIIALKTLQDFVNDEIRPAIGELRCETTALKLGIKLTQHYSELATAFSSNLGTIGEKSLTLQALSSLYSANITEILSTIKKDKSDIYDIIYTEQVKGTVIDVDLEKYMVTLLVKIPILSEIPGVLIYRASSISYNIEGEEWHVAIPNYIINKASSLGGADVTNCIESKLAYICPRDPTQLIPDNQQKCILGDVSKCPVTKVINNLVPKFAFINGGVVANCIASTCTCGTNRIPVNQDRSKGVTFLTYTNCGLIGINGIELYANKRGRDTTWGNQIIKVGPAVSIRPVDISLNLASLTNFLEESKTEL

SEQ ID NO: 217 - protein, 91 aa, synthetic construct; identical to SEQ ID NO: 215

SEQ ID NO: 218 - protein, 365 aa, synthetic construct
FFGAVIGTIALGVATAAQITAGIALAEAREARKDIALIKDSIVKTHNSVEFIQRGIGEQIIALKTLQDFVNDEIRPAIGELRCETTALKLGIKLTQHYSELATAFSSNLGTIGEKSLTLQALSSLYSANITEILSTIKKDKSDIYDIIYTEQVKGTVIDVDLEKYMVTLLVKIPILSEIPGVLIYRASSISYNIEGEEWHVAIPNYIINKASSLGGADVTNCIESKLAYICPRDPTQLIPDNQQKCILGDVSKCPVTKVINNLVPKFAFINGGVVANCIASTCTCGTNRIPVNQDRSKGVTFLTYTNCGLIGINGIELYANKRGRDTTWGNQIIKVGPAVSIRPVDISLNLASVTNFLEESKTEL

SEQ ID NO: 219 - protein, 91 aa, synthetic construct; identical to SEQ ID NO: 215

SEQ ID NO: 220 - protein, 365 aa, synthetic construct
FFGAVIGTIALGVATAAQITAGIALAEAREARKDIALIKDSIVKTHNSVEFIQRGIGEQIIALKTLQDFVNDEIRPAIGELRCETTALKLGIKLTQHYSELATAFSSNLGTIGEKSLTLQALSSLYSANITEILSTIKKDKSDIYDIIYTEQVKGTVIDVDLEKYMVTLLVKIPILSEIPGVLIYRASSISYNIEGEEWHVAIPNYIINKASSLGGADVTNCIESKLAYICPRDPTQLIPDNQQKCILGDVSKCPVTKVINNLVPKFAFINGGVVANCIASTCTCGTNRIPVNQDRSKGVTFLTYTNCGLIGINGIELYANKRGRDTTWGNQIIKVGPAVSIRPVDISLNLASATNFLEEVKTEL

SEQ ID NO: 221 - protein, 91 aa, synthetic construct; identical to SEQ ID NO: 215

SEQ ID NO: 222 - protein, 365 aa, synthetic construct
FFGAVIGTIALGVATAAQITAGIALAEAREARKDIALIKDSIVKTHNSVEFIQRGIGEQIIALKTLQDFVNDEIRPAIGELRCETTALKLGIKLTQHYSELATAFSSNLGTIGEKSLTLQALSSLYSANITEILSTIKKDKSDIYDIIYTEQVKGTVIDVDLEKYMVTLLVKIPILSEIPGVLIYRASSISYNIEGEEWHVAIPNYIINKASSLGGADVTNCIESKLAYICPRDPTQLIPDNQQKCILGDVSKCPVTKVINNLVPKFAFINGGVVANCIASTCTCGTNRIPVNQDRSKGVTFLTYTNCGLIGINGIELYANKRGRDTTWGNQIIKVGPAVSIRPVDISLNLASATNFLEELKTEL

SEQ ID NO: 223 - protein, 91 aa, synthetic construct; identical to SEQ ID NO: 215

SEQ ID NO: 224 - protein, 368 aa, synthetic construct
FFGAVIGTIALGVATAAQITAGIALAEAREARKDIALIKDSIVKTHNSVEFIQRGIGEQIIALKTLQDFVNDEIRPAIGELRCETTALKLGIKLTQHYSELATAFSSNLGTIGEKSLTLQALSSLYSANITEILSTIKKDKSDIYDIIYTEQVKGTVIDVDLEKYMVTLLVKIPILSEIPGVLIYRASSISYNIEGEEWHVAIPNYIINKASSLGGADVTNCIESKLAYICPRDPTQLIPDNQQKCILGDVSKCPVTKVINNLVPKFAFINGGVVANCIASTCTCGTNRIPVNQDRSKGVTFLTYTNCGLIGINGIELYANKRGRDTTWGNQIIKVGPAVSIRPVDISLNLASATNFLEESKTELMKL

SEQ ID NO: 225 - protein, 91 aa, synthetic construct; identical to SEQ ID NO: 215

SEQ ID NO: 226 - protein, 368 aa, synthetic construct
FFGAVIGTIALGVATAAQITAGIALAEAREARKDIALIKDSIVKTHNSVEFIQRGIGEQIIALKTLQDFVNDEIRPAIGELRCETTALKLGIKLTQHYSELATAFSSNLGTIGEKSLTLQALSSLYSANITEILSTIKKDKSDIYDIIYTEQVKGTVIDVDLEKYMVTLLVKIPILSEIPGVLIYRASSISYNIEGEEWHVAIPNYIINKASSLGGADVTNCIESKLAYICPRDPTQLIPDNQQKCILGDVSKCPVTKVINNLVPKFAFINGGVVANCIASTCTCGTNRIPVNQDRSKGVTFLTYTNCGLIGINGIELYANKRGRDTTWGNQIIKVGPAVSIRPVDISLNLASATNFLEESKTELMKV

SEQ ID NO: 227 - protein, 91 aa, synthetic construct; identical to SEQ ID NO: 215

SEQ ID NO: 228 - protein, 365 aa, synthetic construct
FFGAVIGTIALGVATAAQITAGIALAEAREARKDIALIKDSIVKTHNSVEFIQRGIGEQIIALKTLQDFVNDEIRPAIGELRCETTALKLGIKLTQHYSELATAFSSNLGTIGEKSLTLQALSSLYSANITEILSTIKKDKSDIYDIIYTEQVKGTVIDVDLEKYMVTLLVKIPILSEIPGVLIYRASSISYNIEGEEWHVAIPNYIINKASSLGGADVTNCIESKLAYICPRDPTQLIPDNQQKCILGDVSKCPVTKVINNLVPKFAFINGGVVANCIASTCTCGTNRIPVNQDRSKGVTFLTYTNCGLIGINGIELYANKRGRDTTWGNQIIKVGPAVSIRPVDISLNLASLTNFLEEAKTEL

SEQ ID NO: 229 - protein, 91 aa, synthetic construct; identical to SEQ ID NO: 215

SEQ ID NO: 230 - protein, 365 aa, synthetic construct
FFGAVIGTIALGVATAAQITAGIALAEAREARKDIALIKDSIVKTHNSVEFIQRGIGEQIIALKTLQDFVNDEIRPAIGELRCETTALKLGIKLTQHYSELATAFSSNLGTIGEKSLTLQALSSLYSANITEILSTIKKDKSDIYDIIYTEQVKGTVIDVDLEKYMVTLLVKIPILSEIPGVLIYRASSISYNIEGEEWHVAIPNYIINKASSLGGADVTNCIESKLAYICPRDPTQLIPDNQQKCILGDVSKCPVTKVINNLVPKFAFINGGVVANCIASTCTCGTNRIPVNQDRSKGVTFLTYTNCGLIGINGIELYANKRGRDTTWGNQIIKVGPAVSIRPVDISLNLASLTNFLEELKTEL

SEQ ID NO: 231 - protein, 91 aa, synthetic construct; identical to SEQ ID NO: 215

SEQ ID NO: 232 - protein, 365 aa, synthetic construct
FFGAVIGTIALGVATAAQITAGIALAEAREARKDIALIKDSIVKTHNSVEFIQRGIGEQIIALKTLQDFVNDEIRPAIGELRCETAALKLGIKLTQHYSELATAFSSNLGTIGEKSLTLQALSSLYSANITEILSTIKKDKSDIYDIIYTEQVKGTVIDVDLEKYMVTLLVKIPILSEIPGVLIYRASSISYNIEGEEWHVAIPNYIINKASSLGGADVTNCIESKLAYICPRDPTQLIPDNQQKCILGDVSKCPVTKVINNLVPKFAFINGGVVANCIASTCTCGTNRIPVNQDRSKGVTFLTYTNCGLIGINGIELYANKRGRDTTWGNQIIKVGPAVSIRPVDISLNLASATNFLEESKTEL

SEQ ID NO: 233 - protein, 91 aa, synthetic construct; identical to SEQ ID NO: 215

SEQ ID NO: 234 - protein, 365 aa, synthetic construct
FFGAVIGTIALGVATAAQITAGIALAEAREARKDIALIKDSIVKTHNSVEFIQRGIGEQIIALKTLQDFVNDEIRPAIGELRCETTALKLGIKLTQHYSELATAFSSNLGTIGEKSLTLQALSSLYSANITEILSTIKKDKSDIYDIIYTEQVKGTVIDVDLEKYMVTLLVKIPILSEIPGVLIYRASSISYNIEGEEWHVAIPNYIINKASSLGGADVTNCIESKLAYICPRDPTQLIPDNQQKCILGDVSKCPVTKVINNLVPKFAFINGGVVANCIASTCTCGTNRIPVNQDRSKGVTFLTYTNCGLIGINGIELYANKRGRDTTWGNQIIKVGPAVSIRPVDISLNLASATNFLEESKTEL

SEQ ID NO: 235 - protein, 91 aa, synthetic construct
QIPVDKLSNVGVIINEGKLLKIAGSYESRYIVLSLVPSIDLQDGCGTTQIIQYKNLLNRLLIPLKDALDLAESLITITNDTTVTNDNPQTR

SEQ ID NO: 236 - protein, 365 aa, synthetic construct
FFGAVIGTIALGVATAAQITAAIALAEAREARKDIALIKDSIVKTHNSVEFIQRGIGEQIIALKTLQDFVNDEIRPAIGELRCETTALKLGIKLTQHYSELATAFSSNLGTIGEKSLTLQALSSLYSANITEILSTIKKDKSDIYDIIYTEQVKGTVIDVDLEKYMVTLLVKIPILSEIPGVLIYRASSISYNIEGEEWHVAIPNYIINKASSLGGADVTNCIESKLAYICPRDPTQLIPDNQQKCILGDVSKCPVTKVINNLVPKFAFINGGVVANCIASTCTCGTNRIPVNQDRSKGVTFLTYTNCGLIGINGIELYANKRGRDTTWGNQIIKVGPAVSIRPVDISLNLASATNFLEESKTEL

SEQ ID NO: 237 - protein, 91 aa, synthetic construct; identical to SEQ ID NO: 235

SEQ ID NO: 238 - protein, 365 aa, synthetic construct; identical to SEQ ID NO: 234

SEQ ID NO: 239 - protein, 91 aa, synthetic construct
QIPVDKLSNVGVIINEGKLLKIAGSYESRYIVLSLVPSIDLQDGCGTTQIIQYKNLLNRLLIPLKDALDLLESLITITNDTTVTNDNPQTR

SEQ ID NO: 240 - protein, 365 aa, synthetic construct; identical to SEQ ID NO: 236

SEQ ID NO: 241 - protein, 91 aa, synthetic construct; identical to SEQ ID NO: 239

SEQ ID NO: 242 - protein, 365 aa, synthetic construct
FFGAVIGTIALGVATAAQITAGIALAEAREARKDIALIKDSIVKTHNSVEFIQRGIGEQIIALKTLQDFVNDEIRPAIGELRCETTALKLGIKLTQHYSELATAFSSNLGTIGEKSLTLQALSSLYSANITEILSTIKKDKSDIYDIIYTEQVKGTVIDVDLEKYMVTLLVKIPILSEIPGVLIYRASSISYNIEGEEWHVAIPNYIINKASSLGGADVTNCIESKLAYICPRDPTQLIPDNQQKCILGDVSKCPVTKVINNLVPKFAFINGGVVANCIASTCTCGTNRIPVNQDRSKGVTFLTYTNCGLIGINGIELYANKRGRDTTWGNQIIKVGPAVSIRPVDISLNLASVTNFLEEVKTEL

SEQ ID NO: 243 - protein, 91 aa, synthetic construct; identical to SEQ ID NO: 215

SEQ ID NO: 244 - protein, 365 aa, synthetic construct; identical to SEQ ID NO: 236

SEQ ID NO: 245 - protein, 91 aa, synthetic construct; identical to SEQ ID NO: 215

SEQ ID NO: 246 - protein, 368 aa, synthetic construct
FFGAVIGTIALGVATAAQITACIALAEAREARKDIALIKDSIVKTHNSVEFIQRGIGEQIIALKTLQDFVNDEIRPAIGELRCETTALKLGIKLTQHYSELATAFSSNLGTIGEKSLTLQALSSLYSANITEILSTIKKDKSDIYDIIYTEQVKGTVIDVDLEKYMVTLLVKIPILSEIPGVLIYRASSISYNIEGEEWHVAIPNYIINKASSLGGADVTNCIESKLAYICPRDPTQLIPDNQQKCILGDVSKCPVTKVINNLVPKFAFINGGVVANCIASTCTCGTNRIPVNQDRSKGVTFLTYTNCGLIGINGIELYANKRGRDTTWGNQIIKVGPAVSIRPVDISLNLASLTNFLEELKTELMKL

SEQ ID NO: 247 - protein, 91 aa, synthetic construct; identical to SEQ ID NO: 213

SEQ ID NO: 248 - protein, 365 aa, synthetic construct
FFGAVIGTIALGVATAAQITACIALAEAREARKDIALIKDSIVKTHNSVEFIQRGIGEQIIALKTLQDFVNDEIRPAIGELRCETTALKLGIKLTQHYSELATAFSSNLGTIGEKSLTLQALSSLYSANITEILSTIKKDKSDIYDIIYTEQVKGTVIDVDLEKYMVTLLVKIPILSEIPGVLIYRASSISYNIEGEEWHVAIPNYIINKASSLGGADVTNCIESKLAYICPRDPTQLIPDNQQKCILGDVSKCPVTKVINNLVPKFAFINGGVVANCIASTCTCGTNRIPVNQDRSKGVTFLTYTNCGLIGINGIELYANKRGRDTTWGNQIIKVGPAVSIRPVDISLNLASLTNFLEELKTEL

SEQ ID NO: 249 - protein, 91 aa, synthetic construct; identical to SEQ ID NO: 213

SEQ ID NO: 250 - protein, 368 aa, synthetic construct
FFGAVIGTIALGVATAAQITACIALAEAREARKDIALIKDSIVKTHNSVEFIQRGIGEQIIALKTLQDFVNDEIRPAIGELRCETTALKLGIKLTQHYSELATAFSSNLGTIGEKSLTLQALSSLYSANITEILSTIKKDKSDIYDIIYTEQVKGTVIDVDLEKYMVTLLVKIPILSEIPGVLIYRASSISYNIEGEEWHVAIPNYIINKASSLGGADVTNCIESKLAYICPRDPTQLIPDNQQKCILGDVSKCPVTKVINNLVPKFAFINGGVVANCIASTCTCGTNRIPVNQDRSKGVTFLTYTNCGLIGINGIELYANKRGRDTTWGNQIIKVGPAVSIRPVDISLNLASVTNFLEEVKTELMKV

SEQ ID NO: 251 - protein, 91 aa, synthetic construct; identical to SEQ ID NO: 213

SEQ ID NO: 252 - protein, 365 aa, synthetic construct
FFGAVIGTIALGVATAAQITACIALAEAREARKDIALIKDSIVKTHNSVEFIQRGIGEQIIALKTLQDFVNDEIRPAIGELRCETTALKLGIKLTQHYSELATAFSSNLGTIGEKSLTLQALSSLYSANITEILSTIKKDKSDIYDIIYTEQVKGTVIDVDLEKYMVTLLVKIPILSEIPGVLIYRASSISYNIEGEEWHVAIPNYIINKASSLGGADVTNCIESKLAYICPRDPTQLIPDNQQKCILGDVSKCPVTKVINNLVPKFAFINGGVVANCIASTCTCGTNRIPVNQDRSKGVTFLTYTNCGLIGINGIELYANKRGRDTTWGNQIIKVGPAVSIRPVDISLNLASVTNFLEEVKTEL

SEQ ID NO: 253 - protein, 91 aa, synthetic construct; identical to SEQ ID NO: 213

SEQ ID NO: 254 - protein, 368 aa, synthetic construct
FFGAVIGTIALGVATAAQITAAIALAEAREARKDIALIKDSIVKTHNSVEFIQRGIGEQIIALKTLQDFVNDEIRPAIGELRCETTALKLGIKLTQHYSELATAFSSNLGTIGEKSLTLQALSSLYSANITEILSTIKKDKSDIYDIIYTEQVKGTVIDVDLEKYMVTLLVKIPILSEIPGVLIYRASSISYNIEGEEWHVAIPNYIINKASSLGGADVTNCIESKLAYICPRDPTQLIPDNQQKCILGDVSKCPVTKVINNLVPKFAFINGGVVANCIASTCTCGTNRIPVNQDRSKGVTFLTYTNCGLIGINGIELYANKRGRDTTWGNQIIKVGPAVSIRPVDISLNLASLTNFLEELKTELMKL

SEQ ID NO: 255 - protein, 91 aa, synthetic construct; identical to SEQ ID NO: 215

SEQ ID NO: 256 - protein, 365 aa, synthetic construct
FFGAVIGTIALGVATAAQITAAIALAEAREARKDIALIKDSIVKTHNSVEFIQRGIGEQIIALKTLQDFVNDEIRPAIGELRCETTALKLGIKLTQHYSELATAFSSNLGTIGEKSLTLQALSSLYSANITEILSTIKKDKSDIYDIIYTEQVKGTVIDVDLEKYMVTLLVKIPILSEIPGVLIYRASSISYNIEGEEWHVAIPNYIINKASSLGGADVTNCIESKLAYICPRDPTQLIPDNQQKCILGDVSKCPVTKVINNLVPKFAFINGGVVANCIASTCTCGTNRIPVNQDRSKGVTFLTYTNCGLIGINGIELYANKRGRDTTWGNQIIKVGPAVSIRPVDISLNLASLTNFLEELKTEL

SEQ ID NO: 257 - protein, 91 aa, synthetic construct; identical to SEQ ID NO: 215

SEQ ID NO: 258 - protein, 368 aa, synthetic construct; identical to SEQ ID NO: 254

SEQ ID NO: 259 - protein, 91 aa, synthetic construct; identical to SEQ ID NO: 235

SEQ ID NO: 260 - protein, 365 aa, synthetic construct; identical to SEQ ID NO: 256

SEQ ID NO: 261 - protein, 91 aa, synthetic construct; identical to SEQ ID NO: 235

SEQ ID NO: 262 - protein, 368 aa, synthetic construct; identical to SEQ ID NO: 254

SEQ ID NO: 263 - protein, 91 aa, synthetic construct; identical to SEQ ID NO: 239

SEQ ID NO: 264 - protein, 365 aa, synthetic construct; identical to SEQ ID NO: 256

SEQ ID NO: 265 - protein, 91 aa, synthetic construct; identical to SEQ ID NO: 239

SEQ ID NO: 266 - protein, 368 aa, synthetic construct
FFGAVIGTIALGVATAAQITALIALAEAREARKDIALIKDSIVKTHNSVEFIQRGIGEQIIALKTLQDFVNDEIRPAIGELRCETTALKLGIKLTQHYSELATAFSSNLGTIGEKSLTLQALSSLYSANITEILSTIKKDKSDIYDIIYTEQVKGTVIDVDLEKYMVTLLVKIPILSEIPGVLIYRASSISYNIEGEEWHVAIPNYIINKASSLGGADVTNCIESKLAYICPRDPTQLIPDNQQKCILGDVSKCPVTKVINNLVPKFAFINGGVVANCIASTCTCGTNRIPVNQDRSKGVTFLTYTNCGLIGINGIELYANKRGRDTTWGNQIIKVGPAVSIRPVDISLNLASLTNFLEELKTELMKL

SEQ ID NO: 267 - protein, 91 aa, synthetic construct; identical to SEQ ID NO: 235

SEQ ID NO: 268 - protein, 368 aa, synthetic construct
FFGAVIGTIALGVATAAQITAAIALAEAREARKDIALIKDSIVKTHNSVEFIQRGIGEQIIALKTLQDFVNDEIRPAIGELRCETTALKLGIKLTQHYSELATAFSSNLGTIGEKSLTLQALSSLYSANITEILSTIKKDKSDIYDIIYTEQVKGTVIDVDLEKYMVTLLVKIPILSEIPGVLIYRASSISYNIEGEEWHVAIPNYIINKASSLGGADVTNCIESKLAYICPRDPTQLIPDNQQKCILGDVSKCPVTKVINNLVPKFAFINGGVVANCIASTCTCGTNRIPVNQDRSKGVTFLTYTNCGLIGINGIELYANKRGRDTTWGNQIIKVGPAVSIRPVDISLNLASITNFLEEIKTELMKL

SEQ ID NO: 269 - protein, 91 aa, synthetic construct; identical to SEQ ID NO: 239

SEQ ID NO: 270 - protein, 368 aa, synthetic construct
FFGAVIGTIALGVATAAQITAAIALAEAREARKDIALIKDSIVKTHNSVEFIQRGIGEQIIALKTLQDFVNDEIRPAIGELRCETTALKLGIKLTQHYSELATAFSSNLGTIGEKSLTLQALSSLYSANITEILSTIKKDKSDIYDIIYTEQVKGTVIDVDLEKYMVTLLVKIPILSEIPGVLIYRASSISYNIEGEEWHVAIPNYIINKASSLGGADVTNCIESKLAYICPRDPTQLIPDNQQKCILGDVSKCPVTKVINNLVPKFAFINGGVVANCIASTCTCGTNRIPVNQDRSKGVTFLTYTNCGLIGINGIELYANKRGRDTTWGNQIIKVGPAVSIRPVDISLNLASITNFLEEIKTELMKI

SEQ ID NO: 271 - protein, 91 aa, synthetic construct; identical to SEQ ID NO: 239

SEQ ID NO: 272 - protein, 368 aa, synthetic construct
GSGAVIGTIALGVATAAQITACIALAEAREARKDIALIKDSIVKTHNSVEFIQRGIGEQIIALKTLQDFVNDEIRPAIGELRCETTALKLGIKLTQHYSELATAFSSNLGTIGEKSLTLQALSSLYSANITEILSTIKKDKSDIYDIIYTEQVKGTVIDVDLEKYMVTLLVKIPILSEIPGVLIYRASSISYNIEGEEWHVAIPNYIINKASSLGGADVTNCIESKLAYICPRDPTQLIPDNQQKCILGDVSKCPVTKVINNLVPKFAFINGGVVANCIASTCTCGTNRIPVNQDRSKGVTFLTYTNCGLIGINGIELYANKRGRDTTWGNQIIKVGPAVSIRPVDISLNLASLTNFLEELKTELMKL

SEQ ID NO: 273 - protein, 91 aa, synthetic construct; identical to SEQ ID NO: 213

SEQ ID NO: 274 - protein, 368 aa, synthetic construct
GSGAVIGTIALGVATAAQITACIALAEAREARKDIALIKDSIVKTHNSVEFIQRGIGEQIIALKTLQDFVNDEIRPAIGELRCETTALKLGIKLTQHYSELATAFSSNLGTIGEKSLTLQALSSLYSANITEILSTIKKDKSDIYDIIYTEQVKGTVIDVDLEKYMVTLLVKIPILSEIPGVLIYRASSISYNIEGEEWHVAIPNYIINKASSLGGADVTNCIESKLAYICPRDPTQLIPDNQQKCILGDVSKCPVTKVINNLVPKFAFINGGVVANCIASTCTCGTNRIPVNQDRSKGVTFLTYTNCGLIGINGIELYANKRGRDTTWGNQIIKVGPAVSIRPVDISLNLASVTNFLEEVKTELMKV

SEQ ID NO: 275 - protein, 91 aa, synthetic construct; identical to SEQ ID NO: 213

SEQ ID NO: 276 - protein, 368 aa, synthetic construct
GSGAVIGTIALGVATAAQITAAIALAEAREARKDIALIKDSIVKTHNSVEFIQRGIGEQIIALKTLQDFVNDEIRPAIGELRCETTALKLGIKLTQHYSELATAFSSNLGTIGEKSLTLQALSSLYSANITEILSTIKKDKSDIYDIIYTEQVKGTVIDVDLEKYMVTLLVKIPILSEIPGVLIYRASSISYNIEGEEWHVAIPNYIINKASSLGGADVTNCIESKLAYICPRDPTQLIPDNQQKCILGDVSKCPVTKVINNLVPKFAFINGGVVANCIASTCTCGTNRIPVNQDRSKGVTFLTYTNCGLIGINGIELYANKRGRDTTWGNQIIKVGPAVSIRPVDISLNLASLTNFLEELKTELMKL

SEQ ID NO: 277 - protein, 91 aa, synthetic construct; identical to SEQ ID NO: 215

SEQ ID NO: 278 - protein, 367 aa, synthetic construct
GSGAVIGTIALGVATAAQITAAIALAEAREARKDIALIKDSIVKTHNSVEFIQRGIGEQIIALKTLQDFVNDEIRPAIGELRCETTALKLGIKLTQHYSELATAFSSNLGTIGEKSLTLQALSSLYSANITEILSTIKKDKSDIYDIIYTEQVKGTVIDVDLEKYMVTLLVKIPILSEIPGVLIYRASSISYNIEGEEWHVAIPNYIINKASSLGGADVTNCIESKLAYICPRDPTQLIPDNQQKCILGDVSKCPVTKVINNLVPKFAFINGGVVANCIASTCTCGTNRIPVNQDRSKGVTFLTYTNCGLIGINGIELYANKRGRDTTWGNQIIKVGPAVSIRPVDISLNLASLTNFLEELKTELMK

SEQ ID NO: 279 - protein, 91 aa, synthetic construct; identical to SEQ ID NO: 235

SEQ ID NO: 280 - protein, 368 aa, synthetic construct; identical to SEQ ID NO: 276

SEQ ID NO: 281 - protein, 91 aa, synthetic construct; identical to SEQ ID NO: 239

SEQ ID NO: 282 - protein, 368 aa, synthetic construct
GSGAVIGTIALGVATAAQITALIALAEAREARKDIALIKDSIVKTHNSVEFIQRGIGEQIIALKTLQDFVNDEIRPAIGELRCETTALKLGIKLTQHYSELATAFSSNLGTIGEKSLTLQALSSLYSANITEILSTIKKDKSDIYDIIYTEQVKGTVIDVDLEKYMVTLLVKIPILSEIPGVLIYRASSISYNIEGEEWHVAIPNYIINKASSLGGADVTNCIESKLAYICPRDPTQLIPDNQQKCILGDVSKCPVTKVINNLVPKFAFINGGVVANCIASTCTCGTNRIPVNQDRSKGVTFLTYTNCGLIGINGIELYANKRGRDTTWGNQIIKVGPAVSIRPVDISLNLASLTNFLEELKTELMKL

SEQ ID NO: 283 - protein, 91 aa, synthetic construct; identical to SEQ ID NO: 235

SEQ ID NO: 284 - protein, 368 aa, synthetic construct
GSGAVIGTIALGVATAAQITACIALAEAREARKDIALIKDSIVKTHNSVEFIQRGIGEQIIALKTLQDFVNDEIRPAIGELRCETTALKLGIKLTQHYSELATAFSSNLGTIGEKSLTLQALSSLYSANITEILSTIKKDKSDIYDIIYTEQVKGTVIDVDLEKYMVTLLVKIPILSEIPGVLIYRASSISYNIEGEEWHVAIPNYIINKASSLGGADVTNCIESKLAYICPRDPTQLIPDNQQKCILGDVSKCPVTKVINNLVPKFAFINGGVVANCIASTCTCGTNRIPVNQDRSKGVTFLTYTNCGLIGINGIELYANKRGRDTTWGNQIIKVGPAVSIRPVDISLNLASITNFLEEIKTELMKL

SEQ ID NO: 285 - protein, 91 aa, synthetic construct; identical to SEQ ID NO: 213

SEQ ID NO: 286 - protein, 365 aa, synthetic construct
GSGAVIGTIALGVATAAQITACIALAEAREARKDIALIKDSIVKTHNSVEFIQRGIGEQIIALKTLQDFVNDEIRPAIGELRCETTALKLGIKLTQHYSELATAFSSNLGTIGEKSLTLQALSSLYSANITEILSTIKKDKSDIYDIIYTEQVKGTVIDVDLEKYMVTLLVKIPILSEIPGVLIYRASSISYNIEGEEWHVAIPNYIINKASSLGGADVTNCIESKLAYICPRDPTQLIPDNQQKCILGDVSKCPVTKVINNLVPKFAFINGGVVANCIASTCTCGTNRIPVNQDRSKGVTFLTYTNCGLIGINGIELYANKRGRDTTWGNQIIKVGPAVSIRPVDISLNLASITNFLEEIKTEL

SEQ ID NO: 287 - protein, 91 aa, synthetic construct; identical to SEQ ID NO: 213

SEQ ID NO: 288 - protein, 368 aa, synthetic construct
GSGAVIGTIALGVATAAQITACIALAEAREARKDIALIKDSIVKTHNSVEFIQRGIGEQIIALKTLQDFVNDEIRPAIGELRCETTALKLGIKLTQHYSELATAFSSNLGTIGEKSLTLQALSSLYSANITEILSTIKKDKSDIYDIIYTEQVKGTVIDVDLEKYMVTLLVKIPILSEIPGVLIYRASSISYNIEGEEWHVAIPNYIINKASSLGGADVTNCIESKLAYICPRDPTQLIPDNQQKCILGDVSKCPVTKVINNLVPKFAFINGGVVANCIASTCTCGTNRIPVNQDRSKGVTFLTYTNCGLIGINGIELYANKRGRDTTWGNQIIKVGPAVSIRPVDISLNLASITNFLEEIKTELMKI

SEQ ID NO: 289 - protein, 91 aa, synthetic construct; identical to SEQ ID NO: 213

SEQ ID NO: 290 - protein, 365 aa, synthetic construct
GSGAVIGTIALGVATAAQITAAIALAEAREARKDIALIKDSIVKTHNSVEFIQRGIGEQIIALKTLQDFVNDEIRPAIGELRCETTALKLGIKLTQHYSELATAFSSNLGTIGEKSLTLQALSSLYSANITEILSTIKKDKSDIYDIIYTEQVKGTVIDVDLEKYMVTLLVKIPILSEIPGVLIYRASSISYNIEGEEWHVAIPNYIINKASSLGGADVTNCIESKLAYICPRDPTQLIPDNQQKCILGDVSKCPVTKVINNLVPKFAFINGGVVANCIASTCTCGTNRIPVNQDRSKGVTFLTYTNCGLIGINGIELYANKRGRDTTWGNQIIKVGPAVSIRPVDISLNLASLTNFLEELKTEL

SEQ ID NO: 291 - protein, 91 aa, synthetic construct; identical to SEQ ID NO: 215

SEQ ID NO: 292 - protein, 365 aa, synthetic construct
GSGAVIGTIALGVATAAQITAAIALAEAREARKDIALIKDSIVKTHNSVEFIQRGIGEQIIALKTLQDFVNDEIRPAIGELRCETTALKLGIKLTQHYSELATAFSSNLGTIGEKSLTLQALSSLYSANITEILSTIKKDKSDIYDIIYTEQVKGTVIDVDLEKYMVTLLVKIPILSEIPGVLIYRASSISYNIEGEEWHVAIPNYIINKASSLGGADVTNCIESKLAYICPRDPTQLIPDNQQKCILGDVSKCPVTKVINNLVPKFAFINGGVVANCIASTCTCGTNRIPVNQDRSKGVTFLTYTNCGLIGINGIELYANKRGRDTTWGNQIIKVGPAVSIRPVDISLNLASITNFLEEIKTEL

SEQ ID NO: 293 - protein, 91 aa, synthetic construct; identical to SEQ ID NO: 215

SEQ ID NO: 294 - DNA, 1617 bp, synthetic construct
  source 1..1617: mol_type = other DNA
atgctgatctccatcctgctgatcatcaccacaatgatcatggccagccactgccagatcgacatcaccaagctgcagcacgtgggcgtgctggtcaatagccctaagggcatgaagatcagccagaacttcgagacacgctacctgatcctgtctctgatccccaagatcgaggacagcaacagctgcggcgaccagcagatcaagcagtacaagcggctgctggacagactgatcatccctctgtacgacggcctgcggctgcagaaagatgtgatcgtgaccaatcaagagagcaacgagaacacagaccccagaaccgagagattcttcggcggcgtgatcggcacaatcgccctgggagttgctacaagcgcccagattacagccgccgtggctctggtggaagccaagcaggccagaagcgacatcgagaagctgaaagaggccatccgggacaccaacaaggccgtgcagtgcgtgcaaagcagcgtgggcaatctgatctgcgccattaagagcgtgcaggactacgtgaacaaagagatcgtcccctctatcgccagactgggatgtgaagctgccggactgcagctgggaattgccctgacacagcactacagcgagctgaccaacatcttcggcgacaacatcggcagcctgcaagagaagggcattaagctgcagggaatcgccagcctgtaccgcaccaacatcaccgagatcttcaccaccagcaccgtggataagtacgacatctacgacctgctgttcaccgagagcatcaaagtgcgcgtgatcgacgtggacctgaacgactacagcatcaccctgcaagtgcggctgcctctgctgaccagactgctgaacacccagatctacaaggtggacagcatctcctacaacatccagaaccgcgagtggtacatcccactgcctagccacattatgaccaagggcgcctttctcggcggagccgatgtgaaagagtgcatcgaggccttcagcagctacatctgccctagcgatcctggcttcgtgctgaaccacgagatggaaagctgcctgagcggcaacatctctcagtgccctagaaccaccgtgacctccgacatcgtgcccagatacgcctttgtgaatggcggcgtggtggccaactgcatcaccaccacctgtacctgcaacggcatcggcaaccggatcaaccagcctccagatcagggcgtgaagattatcacccacaaagagtgtaacaccatcggcatcaacggcatgctgttcaataccaacaaagagggcaccctggccttctacacccctgacgatatcaccctgaacaacagcgtggccctggatcctatcgacatctccatcgagctgaacaaggccaagagcgacctggaagaaagcaaagagtggatccggcggagcaaccagaagctggatagcatcggaagctggcaccagagcagcaccaccatcatcgtgatcctgattatgatgattatcctgttcatcatcaacattaccatcatcacgatcgccatcaagtactaccggatccagaaacggaaccgcgtggaccagaatgacaagccctacgtgctgacaaacaag

SEQ ID NO: 295 - RNA, 1617 nt, synthetic construct
  source 1..1617: mol_type = other RNA
atgctgatctccatcctgctgatcatcaccacaatgatcatggccagccactgccagatcgacatcaccaagctgcagcacgtgggcgtgctggtcaatagccctaagggcatgaagatcagccagaacttcgagacacgctacctgatcctgtctctgatccccaagatcgaggacagcaacagctgcggcgaccagcagatcaagcagtacaagcggctgctggacagactgatcatccctctgtacgacggcctgcggctgcagaaagatgtgatcgtgaccaatcaagagagcaacgagaacacagaccccagaaccgagagattcttcggcggcgtgatcggcacaatcgccctgggagttgctacaagcgcccagattacagccgccgtggctctggtggaagccaagcaggccagaagcgacatcgagaagctgaaagaggccatccgggacaccaacaaggccgtgcagtgcgtgcaaagcagcgtgggcaatctgatctgcgccattaagagcgtgcaggactacgtgaacaaagagatcgtcccctctatcgccagactgggatgtgaagctgccggactgcagctgggaattgccctgacacagcactacagcgagctgaccaacatcttcggcgacaacatcggcagcctgcaagagaagggcattaagctgcagggaatcgccagcctgtaccgcaccaacatcaccgagatcttcaccaccagcaccgtggataagtacgacatctacgacctgctgttcaccgagagcatcaaagtgcgcgtgatcgacgtggacctgaacgactacagcatcaccctgcaagtgcggctgcctctgctgaccagactgctgaacacccagatctacaaggtggacagcatctcctacaacatccagaaccgcgagtggtacatcccactgcctagccacattatgaccaagggcgcctttctcggcggagccgatgtgaaagagtgcatcgaggccttcagcagctacatctgccctagcgatcctggcttcgtgctgaaccacgagatggaaagctgcctgagcggcaacatctctcagtgccctagaaccaccgtgacctccgacatcgtgcccagatacgcctttgtgaatggcggcgtggtggccaactgcatcaccaccacctgtacctgcaacggcatcggcaaccggatcaaccagcctccagatcagggcgtgaagattatcacccacaaagagtgtaacaccatcggcatcaacggcatgctgttcaataccaacaaagagggcaccctggccttctacacccctgacgatatcaccctgaacaacagcgtggccctggatcctatcgacatctccatcgagctgaacaaggccaagagcgacctggaagaaagcaaagagtggatccggcggagcaaccagaagctggatagcatcggaagctggcaccagagcagcaccaccatcatcgtgatcctgattatgatgattatcctgttcatcatcaacattaccatcatcacgatcgccatcaagtactaccggatccagaaacggaaccgcgtggaccagaatgacaagccctacgtgctgacaaacaag

SEQ ID NO: 296 - DNA, 1617 bp, synthetic construct
  source 1..1617: mol_type = other DNA
atgctgatctccatcctgctgatcatcaccacaatgatcatggccagccactgccagatcgacatcaccaagctgcagcacgtgggcgtgctggtcaatagccctaagggcatgaagatcagccagaacttcgagacacgctacctgatcctgtctctgatccccaagatcgaggacagcaacagctgcggcgaccagcagatcaagcagtacaagcggctgctggacagactgatcatccctctgtacgacggcctgcggctgcagaaagatgtgatcgtgaccaatcaagagagcaacgagaacacagaccccagaaccgagagattcttcggcggcgtgatcggcacaatcgccctgggagttgctacaagcgcccagattacagccgccgtggctctggtggaagccaagcaggccagaagcgacatcgagaagctgaaagaggccatccgggacaccaacaaggccgtgcagtctgtgcaaagcagcgtgggcaatctgatcgtggccattaagagcgtgcaggactacgtgaacaaagagatcgtcccctctatcgccagactgggatgtgaagctgccggactgcagctgggaattgccctgacacagcactacagctgcctgaccaacatcttcggcgacaacatcggcagcctgcaagagaagggcattaagctgcagggaatcgccagctgctaccgcaccaacatcaccgagatcttcaccaccagcaccgtggataagtacgacatctacgacctgctgttcaccgagagcatcaaagtgcgcgtgatcgacgtggacctgaacgactacagcatcaccctgcaagtgcggctgcctctgctgaccagactgctgaacacccagatctacaaggtggacagcatctcctacaacatccagaaccgcgagtggtacatcccactgcctagccacattatgaccaagggcgcctttctcggcggagccgatgtgaaagagtgcatcgaggccttcagcagctacatctgccctagcgatcctggcttcgtgctgaaccacgagatggaaagctgcctgagcggcaacatctctcagtgccctagaaccaccgtgacctccgacatcgtgcccagatacgcctttgtgaatggcggcgtggtggccaactgcatcaccaccacctgtacctgcaacggcatcggcaaccggatcaaccagcctccagatcagggcgtgaagattatcacccacaaagagtgtaacaccatcggcatcaacggcatgctgttcaataccaacaaagagggcaccctggccttctacacccctgacgatatcaccctgaacaacagcgtggccctggatcctatcgacatctccatcgagctgaacaaggccaagagcgacctggaagaaagcaaagagtggatccggcggagcaaccagaagctggatagcatcggaagctggcaccagagcagcaccaccatcatcgtgatcctgattatgatgattatcctgttcatcatcaacattaccatcatcacgatcgccatcaagtactaccggatccagaaacggaaccgcgtggaccagaatgacaagccctacgtgctgacaaacaag

SEQ ID NO: 297 - RNA, 1617 nt, synthetic construct
  source 1..1617: mol_type = other RNA
atgctgatctccatcctgctgatcatcaccacaatgatcatggccagccactgccagatcgacatcaccaagctgcagcacgtgggcgtgctggtcaatagccctaagggcatgaagatcagccagaacttcgagacacgctacctgatcctgtctctgatccccaagatcgaggacagcaacagctgcggcgaccagcagatcaagcagtacaagcggctgctggacagactgatcatccctctgtacgacggcctgcggctgcagaaagatgtgatcgtgaccaatcaagagagcaacgagaacacagaccccagaaccgagagattcttcggcggcgtgatcggcacaatcgccctgggagttgctacaagcgcccagattacagccgccgtggctctggtggaagccaagcaggccagaagcgacatcgagaagctgaaagaggccatccgggacaccaacaaggccgtgcagtctgtgcaaagcagcgtgggcaatctgatcgtggccattaagagcgtgcaggactacgtgaacaaagagatcgtcccctctatcgccagactgggatgtgaagctgccggactgcagctgggaattgccctgacacagcactacagctgcctgaccaacatcttcggcgacaacatcggcagcctgcaagagaagggcattaagctgcagggaatcgccagctgctaccgcaccaacatcaccgagatcttcaccaccagcaccgtggataagtacgacatctacgacctgctgttcaccgagagcatcaaagtgcgcgtgatcgacgtggacctgaacgactacagcatcaccctgcaagtgcggctgcctctgctgaccagactgctgaacacccagatctacaaggtggacagcatctcctacaacatccagaaccgcgagtggtacatcccactgcctagccacattatgaccaagggcgcctttctcggcggagccgatgtgaaagagtgcatcgaggccttcagcagctacatctgccctagcgatcctggcttcgtgctgaaccacgagatggaaagctgcctgagcggcaacatctctcagtgccctagaaccaccgtgacctccgacatcgtgcccagatacgcctttgtgaatggcggcgtggtggccaactgcatcaccaccacctgtacctgcaacggcatcggcaaccggatcaaccagcctccagatcagggcgtgaagattatcacccacaaagagtgtaacaccatcggcatcaacggcatgctgttcaataccaacaaagagggcaccctggccttctacacccctgacgatatcaccctgaacaacagcgtggccctggatcctatcgacatctccatcgagctgaacaaggccaagagcgacctggaagaaagcaaagagtggatccggcggagcaaccagaagctggatagcatcggaagctggcaccagagcagcaccaccatcatcgtgatcctgattatgatgattatcctgttcatcatcaacattaccatcatcacgatcgccatcaagtactaccggatccagaaacggaaccgcgtggaccagaatgacaagccctacgtgctgacaaacaag

SEQ ID NO: 298 - DNA, 1617 bp, synthetic construct
  source 1..1617: mol_type = other DNA
atgctgatctccatcctgctgatcatcaccacaatgatcatggccagccactgccagatcgacatcaccaagctgcagcacgtgggcgtgctggtcaatagccctaagggcatgaagatcagccagaacttcgagacacgctacctgatcctgtctctgatccccaagatcgaggacagcaacagctgcggcgaccagcagatcaagcagtacaagcggctgctggacagactgatcatccctctgtacgacggcctgcggctgcagaaagatgtgatcgtgaccaatcaagagagcaacgagaacacagaccccagaaccgagagattcttcggcggcgtgatcggcacaatcgccctgggagttgctacaagcgcccagattacagccgccgtggctctggtggaagccaagcaggccagaagcgacatcgagaagctgaaagaggccatccgggacaccaacaaggccgtgcagtctgtgcaaagcagcgtgggcaatctgatcgtggccattaagagcgtgcaggactacgtgaacaaagagatcgtcccctctatcgccagactgggatgtgaagctgccggactgcagctgggaattgccctgacacagcactacagcgagctgaccaacatcttcggcgacaacatcggcagcctgcaagagaagggcattaagctgcagggaatcgccagcctgtaccgcaccaacatcaccgagatcttcaccaccagcaccgtggataagtacgacatctacgacctgctgttcaccgagagcatcaaagtgcgcgtgatcgacgtggacctgaacgactacagcatcaccctgcaagtgcggctgcctctgctgaccagactgctgaacacccagatctacaaggtggacagcatctcctacaacatccagaaccgcgagtggtacatcccactgcctagccacattatgaccaagggcgcctttctcggcggagccgatgtgaaagagtgcatcgaggccttcagcagctacatctgccctagcgatcctggcttcgtgctgaaccacgagatggaaagctgcctgagcggcaacatctctcagtgccctagaaccaccgtgacctccgacatcgtgcccagatacgcctttgtgaatggcggcgtggtggccaactgcatcaccaccacctgtacctgcaacggcatcggcaaccggatcaaccagcctccagatcagggcgtgaagattatcacccacaaagagtgtaacaccatcggcatcaacggcatgctgttcaataccaacaaagagggcaccctggccttctacacccctgacgatatcaccctgaacaacagcgtggccctggatcctatcgacatctccatcgagctgaacaagctcaagagcgacctggaagaactgaaagagtggatccggcggagcaaccagaagctggatagcatcggaagctggcaccagagcagcaccaccatcatcgtgatcctgattatgatgattatcctgttcatcatcaacattaccatcatcacgatcgccatcaagtactaccggatccagaaacggaaccgcgtggaccagaatgacaagccctacgtgctgacaaacaag

SEQ ID NO: 299 - RNA, 1617 nt, synthetic construct
  source 1..1617: mol_type = other RNA
atgctgatctccatcctgctgatcatcaccacaatgatcatggccagccactgccagatcgacatcaccaagctgcagcacgtgggcgtgctggtcaatagccctaagggcatgaagatcagccagaacttcgagacacgctacctgatcctgtctctgatccccaagatcgaggacagcaacagctgcggcgaccagcagatcaagcagtacaagcggctgctggacagactgatcatccctctgtacgacggcctgcggctgcagaaagatgtgatcgtgaccaatcaagagagcaacgagaacacagaccccagaaccgagagattcttcggcggcgtgatcggcacaatcgccctgggagttgctacaagcgcccagattacagccgccgtggctctggtggaagccaagcaggccagaagcgacatcgagaagctgaaagaggccatccgggacaccaacaaggccgtgcagtctgtgcaaagcagcgtgggcaatctgatcgtggccattaagagcgtgcaggactacgtgaacaaagagatcgtcccctctatcgccagactgggatgtgaagctgccggactgcagctgggaattgccctgacacagcactacagcgagctgaccaacatcttcggcgacaacatcggcagcctgcaagagaagggcattaagctgcagggaatcgccagcctgtaccgcaccaacatcaccgagatcttcaccaccagcaccgtggataagtacgacatctacgacctgctgttcaccgagagcatcaaagtgcgcgtgatcgacgtggacctgaacgactacagcatcaccctgcaagtgcggctgcctctgctgaccagactgctgaacacccagatctacaaggtggacagcatctcctacaacatccagaaccgcgagtggtacatcccactgcctagccacattatgaccaagggcgcctttctcggcggagccgatgtgaaagagtgcatcgaggccttcagcagctacatctgccctagcgatcctggcttcgtgctgaaccacgagatggaaagctgcctgagcggcaacatctctcagtgccctagaaccaccgtgacctccgacatcgtgcccagatacgcctttgtgaatggcggcgtggtggccaactgcatcaccaccacctgtacctgcaacggcatcggcaaccggatcaaccagcctccagatcagggcgtgaagattatcacccacaaagagtgtaacaccatcggcatcaacggcatgctgttcaataccaacaaagagggcaccctggccttctacacccctgacgatatcaccctgaacaacagcgtggccctggatcctatcgacatctccatcgagctgaacaagctcaagagcgacctggaagaactgaaagagtggatccggcggagcaaccagaagctggatagcatcggaagctggcaccagagcagcaccaccatcatcgtgatcctgattatgatgattatcctgttcatcatcaacattaccatcatcacgatcgccatcaagtactaccggatccagaaacggaaccgcgtggaccagaatgacaagccctacgtgctgacaaacaag

SEQ ID NO: 300 - protein, 539 aa, synthetic construct
MLISILLIITTMIMASHCQIDITKLQHVGVLVNSPKGMKISQNFETRYLILSLIPKIEDSNSCGDQQIKQYKRLLDRLIIPLYDGLRLQKDVIVTNQESNENTDPRTERFFGGVIGTIALGVATSAQITAAVALVEAKQARSDIEKLKEAIRDTNKAVQSVQSSVGNLIVAIKSVQDYVNKEIVPSIARLGCEAAGLQLGIALTQHYSELTNIFGDNIGSLQEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALDPIDISIELNKAKSDLEESKEWIRRSNQKLDSIGSWHQSSTTIIVILIMMIILFIINITIITIAIKYYRIQKRNRVDQNDKPYVLTNK

SEQ ID NO: 301 - protein, 539 aa, synthetic construct
MLISILSIITTMIMASHCQIDITKLQHVGVLVNSPKGMKISQNFETRYLILSLIPKIEDSNSCGDQQIKQYKRLLDRLIIPLYDGLRLQKDVIVTNQESNENTDPRTERFFGGVIGTIALGVATSAQITAAVALVEAKQARSDIEKLKEAIRDTNKAVQSVQSSIGNLIVAIKSVQDYVNKEIVPSIARLGCEAAGLQLGIALTQHYSELTNIFGDNIGSLQEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALDPIDISIELNKAKSDLEESKEWIRRSNQKLDSIGSWHQSSTTIIVILIMMIILFIINITIITIAIKYYRIQKRNRVDQNDKPYVLTNK

SEQ ID NO: 302 - protein, 539 aa, synthetic construct; identical to SEQ ID NO: 301

SEQ ID NO: 303 - protein, 539 aa, synthetic construct
MLISTLSIITTMIMASHCQIDITKLQHVGVLVNSPKGMKISQNFETRYLILSLIPKIEDSNSCGDQQIKQYKRLLDRLIIPLYDGLRLQKDVIVTNQESNENTDPRTERFFGGVIGTIALGVATSAQITAAVALVEAKQARSDIEKLKEAIRDTNKAVQSVQSSIGNLIVAIKSVQDYVNKEIVPSIARLGCEAAGLQLGIALTQHYSELTNIFGDNIGSLQEKGIKLQGIASLYRTNITEIFTTSTVDKYDIYDLLFTESIKVRVIDVDLNDYSITLQVRLPLLTRLLNTQIYKVDSISYNIQNREWYIPLPSHVMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALDPIDISIELNKAKSDLEESKKWIRRSNQKLDSIGSWHQSSTIIIIILIMMIILFIINITIITIAIKYYRIQKRNRVDQNDKPYVLTNK

SEQ ID NO: 304 - protein, 539 aa, synthetic construct
MPISILLIITTMIMASHCQIDITKLQHVGVLVNSPKGMKISQNFETRYLILSLIPKIEDSNSCGDQQIKQYKRLLDRLIIPLYDGLRLQKDVIVTNQESNENTDPRTERFFGGVIGTIALGVATSAQITAAVALVEAKQARSDIEKLKEAIRDTNKAVQSVQSSVGNLIVAIKSVQDYVNKEIVPSIARLGCEAAGLQLGIALTQHYSELTNIFGDNIGSLQEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFILNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALDPIDISIELNKAKSDLEESKEWIRRSNQKLDSIGSWHQSSTTIIVILIMMIILFIINITIITIAIKYYRIQKRNRMDQNDKPYVLTNK

SEQ ID NO: 305 - protein, 536 aa, synthetic construct
MLISILLIITTMIMASHCQIDITKLQHVGVLVNSPKGMKISQNFETRYLILSLIPKIEDSNSCGDQQIKQYKRLLDRLIIPLYDGLRLQKDVIVTNQESNENTDPRTERFFGGVIGTIALGVATSAQITAAVALVEAKQARSDIEKLKEAIRDTNKAVQSVQSSVGNLIVAIKSVQDYVNKEIVPSIARLGCEAAGLQLGIALTQHYSELTNIFGDNIGSLQEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALDPIDISIELNKAKSDLEESKEWIRRSNQKLGGGSGYIPEAPRDGQAYVRKDGEWVLLSTFLGRSLEVLFQGPGSAWSHPQFEKAG

SEQ ID NO: 306 - protein, 372 aa, synthetic construct
FFGGVIGTIALGVATSAQITAAVALVEAKQARSDIEKLKEAIRDTNKAVQSVQSSVGNLIVAIKSCQDYVNKEIVPSIARLGCEAAGLQLGICLTQHYSELTNIFGDNIGSLQEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALDPIDISIELNKAKSDLEESKEWIRRSNQKL

SEQ ID NO: 307 - protein, 91 aa, synthetic construct
QIDITKLQHVGVLVNSPKGMKISQNFETRYLILSLIPKIEDSNSCGDQQIKQYKRLLDRLIIPLYDGLRLQKDVIVTNQESNENTDPRTER

SEQ ID NO: 308 - protein, 372 aa, synthetic construct
FFGGVIGTIALGVATSAQITAAVALVEAKQARSDIEKLKEAIRDTNKAVQCVQSSVGNLICAIKSVQDYVNKEIVPSIARLGCEAAGLQLGIALTQHYSELTNIFGDNIGSLQEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALDPIDISIELNKAKSDLEESKEWIRRSNQKL

SEQ ID NO: 309 - protein, 91 aa, synthetic construct; identical to SEQ ID NO: 307

SEQ ID NO: 310 - protein, 372 aa, synthetic construct
FFGGVIGTIALGVATSAQITAAVALVEAKQARSDIEKLKEAIRDTNKAVQSVQSPVGNLIVAIKSVQDYVNKEIVPSIARLGCEAAGLQLGIALTQHYSELTNIFGDNIGSLQEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALDPIDISIELNKAKSDLEESKEWIRRSNQKL

SEQ ID NO: 311 - protein, 91 aa, synthetic construct; identical to SEQ ID NO: 307

SEQ ID NO: 312 - protein, 372 aa, synthetic construct
FFGGVIGTIALGVATSAQITAAVALVEAKQARSDIEKLKEAIRDTNKAVQSVQSSVGNLIVAIKSVQDYVNKEIVPSIARLGCEAAALQLGIALTQHYSELTNIFGDNIGSLQEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALDPIDISIELNKAKSDLEESKEWIRRSNQKL

SEQ ID NO: 313 - protein, 91 aa, synthetic construct; identical to SEQ ID NO: 307

SEQ ID NO: 314 - protein, 372 aa, synthetic construct
FFGGVIGTIALGVATSAQITAAVALVEAKQARSDIEKLKEAIRDTNKAVQSVQSSVGNLIVAIKSVQDYVNKEIVPSIARLGCEAAGLQLGIALTQHYSELTNIFGDNIPSLQEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALDPIDISIELNKAKSDLEESKEWIRRSNQKL

SEQ ID NO: 315 - protein, 91 aa, synthetic construct; identical to SEQ ID NO: 307

SEQ ID NO: 316 - protein, 372 aa, synthetic construct
FFGGVIGTIALGVATSAQITAAVALVEAKQARSDIEKLKEAIRDTNKAVQSVQSSVGNLIVAIKSVQDYVNKEIVPSIARLGCEAAGLQLGIALTQHYSELTNIFGDNIGSLQEKGIKLQAIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALDPIDISIELNKAKSDLEESKEWIRRSNQKL

SEQ ID NO: 317 - protein, 91 aa, synthetic construct; identical to SEQ ID NO: 307

SEQ ID NO: 318 - protein, 372 aa, synthetic construct
FFGGVIGTIALGVATSAQITAAVALVEAKQARSDIEKLKEAIRDTNKAVQSVQSSVGNLIVAIKSVQDYVNKLIVPSIARLGCEAAGLQLGIALTQHYSELTNIFGDNIGSLQEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALDPIDISIELNKAKSDLEESKEWIRRSNQKL

SEQ ID NO: 319 - protein, 91 aa, synthetic construct; identical to SEQ ID NO: 307

SEQ ID NO: 320 - protein, 372 aa, synthetic construct
FFGGVIGTIALGVATSAQITAAVALVEAKQARSDIEKLKEAIRDTNKAVQSVQSSVGNLIVAIKSVQDYVNKEIVPSIARLGCEAAGLQLGIALTQHYSELTNIFGDNIGSLQEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALDPIDISIELNKAKSDLEEAKEWIRRSNQKL

SEQ ID NO: 321 - protein, 91 aa, synthetic construct; identical to SEQ ID NO: 307

SEQ ID NO: 322 - protein, 372 aa, synthetic construct
FFGGVIGTIALGVATSAQITAAVALVEAKQARSDIEKLKEAIRDTNKAVQSVQSSVGNLIVAIKSVQDYVNKEIVPSIARLGCEAAGLQLGIALTQHYSELTNIFGDNIGSLQEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALDPIDISIELNKAKSDLEESKEWIRRANQKL

SEQ ID NO: 323 - protein, 91 aa, synthetic construct; identical to SEQ ID NO: 307

SEQ ID NO: 324 - protein, 372 aa, synthetic construct
FFGGVIGTIALGVATSAQITAAVALVEAKQARSDIEKLKEAIRDTNKAVQSVQSSVGNLIVAIKSVQDYVNKEIVPSIARLGCEAAGLQLGIALTQHYSELTNIFGDNIGSLQEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALDPIDISIELNKAKSDLEEAKEWIRRANQKL

SEQ ID NO: 325 - protein, 91 aa, synthetic construct; identical to SEQ ID NO: 307

SEQ ID NO: 326 - protein, 372 aa, synthetic construct
FFGGVIGTIALGVATSAQITAAVALVEAKQARSDIEKLKEAIRDTNKAVQSVQSSVGNLIVAIKSVQDYVNKEIVPSIARLGCEAAGLQLGIALTQHYSELTNIFGDNIGSLQEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALDPIDISIELNKLKSDLEESKEWIRRSNQKL

SEQ ID NO: 327 - protein, 91 aa, synthetic construct; identical to SEQ ID NO: 307

SEQ ID NO: 328 - protein, 372 aa, synthetic construct
FFGGVIGTIALGVATSAQITAAVALVEAKQARSDIEKLKEAIRDTNKAVQSVQSSVGNLIVAIKSVQDYVNKEIVPSIARLGCEAAGLQLGIALTQHYSELTNIFGDNIGSLQEKGIKLQAIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALDPIDISIELNKAKSDLEEAKEWIRRANQKL

SEQ ID NO: 329 - protein, 91 aa, synthetic construct; identical to SEQ ID NO: 307

SEQ ID NO: 330 - protein, 372 aa, synthetic construct
FFGGVIGTIALGVATSAQITAAVALVEAKQARSDIEKLKEAIRDTNKAVQSVQSSVGNLIVAIKSVQDYVNKEIVPSIARLGCEAAGLQLGIALTQHYSELTNIFGDNIGSLQEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALDPIDISIELNKLKSDLEEAKEWIRRANQKL

SEQ ID NO: 331 - protein, 91 aa, synthetic construct; identical to SEQ ID NO: 307

SEQ ID NO: 332 - protein, 372 aa, synthetic construct
FFGGVIGTIALGVATSAQITAAVALVEAKQARSDIEKLKEAIRDTNKAVQSVQSSVGNLIVAIKSVQDYVNKEIVPSIARLGCEAAGLQLGIALTQHYSELTNIFGDNIGSLQEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALDPISISIELNKAKSDLEESKEWIRRSNQKL

SEQ ID NO: 333 - protein, 91 aa, synthetic construct; identical to SEQ ID NO: 307

SEQ ID NO: 334 - protein, 372 aa, synthetic construct
FFGGVIGTIALGVATSAQITAAVALVEAKQARSDIEKLKEAIRDTNKAVQSVQSSVGNLIVAIKSVQDYVNKEIVPSIARLGCEAAGLQLGIALTQHYSELTNIFGDNIGSLQEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALDPIDISIELNKAKSDLEESKEWIRRSNQKL

SEQ ID NO: 335 - protein, 91 aa, synthetic construct
QIDITKLQHVGVLVNSPKGMKISQNFETRYLILSLIPKIEDSNSCGDQQIKQYKRLLDRLIIPLYDGLRLQKDVIVTNQESNENTDPGSAS

SEQ ID NO: 336 - protein, 372 aa, synthetic construct
FFGGVIGTIALGVATSAQITAAVALVEAKQARSDIEKLKEAIRDTNKAVQSVCSSVGNCIVAIKSVQDYVNKEIVPSIARLGCEAAGLQLGIALTQHYSELTNIFGDNIGSLQEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALDPIDISIELNKAKSDLEEAKEWIRRANQKL

SEQ ID NO: 337 - protein, 91 aa, synthetic construct; identical to SEQ ID NO: 307

SEQ ID NO: 338 - protein, 372 aa, synthetic construct
FFGGVIGTIALGVATSAQITAAVALVEAKQARSDIEKLKEAIRDTNKAVQCVQSSVGNLICAIKSVQDYVNKEIVPSIARLGCEAAGLQLGIALTQHYSELTNIFGDNIGSLQEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALDPIDISIELNKAKSDLEEAKEWIRRANQKL

SEQ ID NO: 339 - protein, 91 aa, synthetic construct; identical to SEQ ID NO: 307

SEQ ID NO: 340 - protein, 372 aa, synthetic construct
FFGGVIGTIALGVATSAQITAAVALVEAKQARSDIEKLKEAIRDTNKAVQSVCSSVGNCIVAIKSVQDYVNKEIVPSIARLGCEAAGLQLGIALTQHYSELTNIFGDNIGSLQEKGIKLQAIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALDPIDISIELNKVKSDLEESKEWYRRSNQKL

SEQ ID NO: 341 - protein, 91 aa, synthetic construct; identical to SEQ ID NO: 307

SEQ ID NO: 342 - protein, 372 aa, synthetic construct
FFGGVIGTIALGVATSAQITAAVALVEAKQARSDIEKLKEAIRDTNKAVQSVCSSVGNCIVAIKSVQDYVNKEIVPSIARLGCEAAGLQLGIALTQHYSELTNIFGDNIGSLQEKGIKLQAIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALDPIDISIELNKAKSDLEEAKEWIRRANQKL

SEQ ID NO: 343 - protein, 91 aa, synthetic construct; identical to SEQ ID NO: 307

SEQ ID NO: 344 - protein, 372 aa, synthetic construct
FFGGVIGTIALGVATSAQITAAVALVEAKQARSDIEKLKEAIRDTNKAVQSVCSSVGNCIVAIKSVQDYVNKEIVPSIARLGCEAAGLQLGIALTQHYSELTNIFGDNIGSLQEKGIKLQAIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALDPIDISIELNKLKSDLEESKEWIRRSNQKL

SEQ ID NO: 345 - protein, 91 aa, synthetic construct; identical to SEQ ID NO: 307

SEQ ID NO: 346 - protein, 372 aa, synthetic construct
FFGGVIGTIALGVATSAQITAAVALVEAKQARSDIEKLKEAIRDTNKAVQSVCSSVGNCIVAIKSVQDYVNKEIVPSIARLGCEAAGLQLGIALTQHYSELTNIFGDNIGSLQEKGIKLQAIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALDPIDISIELNKLKSDLEEAKEWIRRANQKL

SEQ ID NO: 347 - protein, 91 aa, synthetic construct; identical to SEQ ID NO: 307

SEQ ID NO: 348 - protein, 372 aa, synthetic construct
FFGGVIGTIALGVATSAQITAAVALVEAKQARSDIEKLKEAIRDTNKAVQCVQSSVGNLICAIKSVQDYVNKEIVPSIARLGCEAAGLQLGIALTQHYSELTNIFGDNIGSLQEKGIKLQAIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALDPIDISIELNKVKSDLEESKEWYRRSNQKL

SEQ ID NO: 349 - protein, 91 aa, synthetic construct; identical to SEQ ID NO: 307

SEQ ID NO: 350 - protein, 372 aa, synthetic construct
FFGGVIGTIALGVATSAQITAAVALVEAKQARSDIEKLKEAIRDTNKAVQCVQSSVGNLICAIKSVQDYVNKEIVPSIARLGCEAAGLQLGIALTQHYSELTNIFGDNIGSLQEKGIKLQAIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALDPIDISIELNKAKSDLEEAKEWIRRANQKL

SEQ ID NO: 351 - protein, 91 aa, synthetic construct; identical to SEQ ID NO: 307

SEQ ID NO: 352 - protein, 372 aa, synthetic construct
FFGGVIGTIALGVATSAQITAAVALVEAKQARSDIEKLKEAIRDTNKAVQCVQSSVGNLICAIKSVQDYVNKEIVPSIARLGCEAAGLQLGIALTQHYSELTNIFGDNIGSLQEKGIKLQAIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALDPIDISIELNKLKSDLEESKEWIRRSNQKL

SEQ ID NO: 353 - protein, 91 aa, synthetic construct; identical to SEQ ID NO: 307

SEQ ID NO: 354 - protein, 372 aa, synthetic construct
FFGGVIGTIALGVATSAQITAAVALVEAKQARSDIEKLKEAIRDTNKAVQCVQSSVGNLICAIKSVQDYVNKEIVPSIARLGCEAAGLQLGIALTQHYSELTNIFGDNIGSLQEKGIKLQAIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALDPIDISIELNKLKSDLEEAKEWIRRANQKL

SEQ ID NO: 355 - protein, 91 aa, synthetic construct; identical to SEQ ID NO: 307

SEQ ID NO: 356 - protein, 372 aa, synthetic construct
FFGGVIGTIALGVATSAQITAAVALVEAKQARSDIEKLKEAIRDTNKAVQCVQSSVGNLICAIKSVQDYVNKEIVPSIARLGCEAAGLQLGIALTQHYSELTNIFGDNIGSLQEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALDPIDISIELNKLKSDLEESKEWIRRSNQKL

SEQ ID NO: 357 - protein, 91 aa, synthetic construct; identical to SEQ ID NO: 307

SEQ ID NO: 358 - protein, 124 aa, synthetic construct
EVQLVESGGGLVKPGGSLRLSCAASGFTFSSYSMNWVRQAPGKGLEWVSSISASSSYSDYADSAKGRFTISRDNAKTSLFLQMNSLRAEDTAIYFCARARATGYSSITPYFDIWGQGTLVTVSS

SEQ ID NO: 359 - protein, 110 aa, synthetic construct
QSVVTQTPSVSGAPGQRVTISCTGSSSNIGAGYDVHWYQQLPGTAPKLLIYDNNNRPSGVPDRFSASKSGTSASLAITGLQAEDEADYYCQSYDRNLSGVFGTGTKVTVL

SEQ ID NO: 360 - protein, 123 aa, synthetic construct
QVQLQQSGAEVKKPGASVKVSCKASGYTFTSYGISWVRQAPGQGLEWMGWISGYNGNTNYAQTFQGTFTMTTDTSTSTAYMELRRLRSGDTAVYYCARDRYYASGSYNGMDVWGQGTTVTVSS

SEQ ID NO: 361 - protein, 110 aa, synthetic construct
QSALTQPPSASGAPGQRVTVSCSGSSSNVGSSSVYWYQQLPGTAPKLLIYRNNQRPSGVPDRFSGSKSGTSASLAISGLRSEDEADYYCAAWDDSLSGWVFGGGTKLTVL

SEQ ID NO: 362 - protein, 122 aa, synthetic construct
QVQLVQSGAEVKKPGSSVKVSCEASGGTFSNYVISWVRQAPGQGLEWMGGIIPVFATAKYAQKFQGRVTIIADQSTNTAYLELSSLRSDDTAVYFCASVDSGGTWWDAFAIWGQGTMVTVSS

SEQ ID NO: 363 - protein, 110 aa, synthetic construct
QAVVTQPRSVSGSPGQAVTISCTGTSSDVGGYNYVSWYQHHPGKAPKLIIYDVTKRPSGVPDRFSGSKSGNTASLTISGLQTEYEADYYCCSYAGSYTLVFGGGTKLTVL

SEQ ID NO: 364 - protein, 221 aa, synthetic construct
VQLQESGPGLVKPSETLSLTCTVSGGSVSSYYWSWIRQPPGKGLEWIGNIYYSGTTKYNPSLKSRVTISVDVSKNQFSLNLISVTAADTAVYFCARQVKSGWFVQPFDYWGQGALVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPK

SEQ ID NO: 365 - protein, 207 aa, synthetic construct
QMTQSPPSLSAYVGDRVTITCRASQAIANYLAWFQQKPGKAPKSLIYAASTLQSGVPSRFSGSGSGTDFTLTISSLQPEDFATYYCHQYNTYPITFGQGTRLEIKRRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKS

SEQ ID NO: 366 - protein, 368 aa, synthetic construct; identical to SEQ ID NO: 254

SEQ ID NO: 367 - protein, 91 aa, synthetic construct; identical to SEQ ID NO: 215

SEQ ID NO: 368 - protein, 368 aa, synthetic construct
FFGAVIGTIALGVATAAQITAAIALAEAREARKDIALIKDSIVKTHNSVEFIQRGIGEQIIALKTLQDFVNDEIRPAIGELRCETTALKLGIKLTQHYSELATAFSSNLGTIGEKSLTLQALSSLYSANITEILSTIKKDKSDIYDIIYTEQVKGTVIDVDLEKYMVTLLVKIPILSEIPGVLIYRASSISYNIEGEEWHVAIPSYIINKASSLGGADVTNCIESKLAYICPRDPTQLIPDNQQKCILGDVSKCPVTKVINNLVPKFAFINGGVVANCIASTCTCGTNRIPVNQDRSKGVTFLTYTNCGLIGINGIELYANKRGRDTTWGNQIIKVGPAVSIRPVDISLNLASLTNFLEELKTELMRL

SEQ ID NO: 369 - protein, 91 aa, synthetic construct
QIPIDKLSNVGVIINEGKLLKIAGSYESRYIVLSLVPSIDLQDGCGTTQIIQYKNLLNRLLIPLKDALDLQESLITITNDTTVTNDNPQTR

SEQ ID NO: 370 - protein, 368 aa, synthetic construct
FFGAVIGTIALGVATAAQITAAIALAEAREARKDIALIKDSIVKTHNSVELIQRGIGEQIIALKTLQDFVNDEIRPAIGELRCETTALKLGIKLTQHYSELATAFSSNLGTIGEKSLTLQALSSLYSANITEILSTTKKDKSDIYDIIYTEQVKGTVIDVDLEKYMVTLLVKIPILSEIPGVLIYRASSISYNIEGEEWHVAIPNYIINKASSLGGADVTNCIESKLAYICPRDPTQLIPDNQQKCILGDVSKCPVTKVINNLVPKFAFINGGVVANCIASTCTCGTNRIPVNQDRSRGVTFLTYTNCGLIGINGIELYANKRGRDTTWGNQIIKVGPAVSIRPVDISLNLASLTNFLEELKTELMKL

SEQ ID NO: 371 - protein, 91 aa, synthetic construct
QIPVEKLSNVGVIINEGKLLKIAGSYESRYIVLSLVPSIDLQDGCGTTQIIQYKNLLNRLLIPLKDALDLQESLITITNDTTVTNDNPQTR

SEQ ID NO: 372 - protein, 368 aa, synthetic construct
FFGAVIGTIALGVATAAQITAAIALAEAREARKDIALIKDSIVKTHNSVEFIQRGIGEQIIALKTLQDFVNDEIRPAIGELRCETTALKLGIKLTQHYSELATAFSSNLGTIGEKSLTLQALSSLYSANITEILSTIKKDKSDIYDIIYTEQVKGTVIDVDLEKYMVTLLVKIPILSEIPGVLIYRASSISYNIEGEEWHVAIPNYIISKASSLGGADVTSCIESKLAYICPRDPTQLIPDNQQKCILGDVSKCPVTKVINNLVPKFAFINGGVVANCIASTCTCGTNRIPVNQDRSKGVTFLTYTNCGLIGINGIELYANKRGRDTTWGNQIIKVGPAVSIRPVDISLNLASLTNFLEELKTELMKL

SEQ ID NO: 373 - protein, 91 aa, synthetic construct; identical to SEQ ID NO: 215

SEQ ID NO: 374 - protein, 368 aa, synthetic construct
FFGAVIGTIALGVATAAQITAAIALAEAREARKDIALIKDSIVKTHNSVEFIQRGIGEQIIALKTLQDFVNDEIRPAIGELRCETTALKLGIKLTQHYSELATAFSSNLGTIGEKSLTLQALSSLYSANITEILSTIKKDKSDIYDIIYTEQVKGTVIDVDLEKYMVTLLVKIPILSEIPGVLIYRASSISYNIEGEEWHVAIPNYIISKASSLGGADVTNCIESKLAYICPRDPTQLIPDNQQKCILGDVSKCPVTKVINNLVPKFAFINGGVVANCIASTCTCGTNRIPVNQDRSKGVTFLTYTNCGLIGINGIELYANKRGRDTTWGNQIIKVGPAVSIRPVDISLNLASLTNFLEELKTELMKL

SEQ ID NO: 375 - protein, 91 aa, synthetic construct; identical to SEQ ID NO: 215

SEQ ID NO: 376 - protein, 372 aa, synthetic construct; identical to SEQ ID NO: 328

SEQ ID NO: 377 - protein, 91 aa, synthetic construct; identical to SEQ ID NO: 307

SEQ ID NO: 378 - protein, 372 aa, synthetic construct
FFGGVIGTIALGVATSAQITAAVALVEAKQARSDIEKLKEAIRDTNKAVQSVQSSVGNLIVAIKSVQDYVNNEIVPSITRLGCEAAGLQLGIALTQHYSELTNIFGDNIGSLQEKGIKLQAIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALDPIDISIELNKAKSDLEEAKEWIRRANQKL

SEQ ID NO: 379 - protein, 91 aa, synthetic construct; identical to SEQ ID NO: 307

SEQ ID NO: 380 - protein, 372 aa, synthetic construct
FFGGVIGTIALGVATSAQITAAVALVEAKQARSDIEKLKEAIRDTNKAVQSVQSSIGNLIVAIKSVQDYVNKEIVPSIARLGCEAAGLQLGIALTQHYSELTNIFGDNIGSLQEKGIKLQAIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALDPIDISIELNKAKSDLEEAKEWIRRANQKL

SEQ ID NO: 381 - protein, 91 aa, synthetic construct; identical to SEQ ID NO: 307

SEQ ID NO: 382 - protein, 372 aa, synthetic construct
FFGGVIGTIALGVATSAQITAAVALVEAKQARSDIEKLKEAIRDTNKAVQSVQSSIGNLIVAIKSVQDYVNKEIVPSIARLGCEAAGLQLGIALTQHYSELTNIFGDNIGSLQEKGIKLQAIASLYRTNITEIFTTSTVDKYDIYDLLFTESIKVRVIDVDLNDYSITLQVRLPLLTRLLNTQIYKVDSISYNIQNREWYIPLPSHVMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALDPIDISIELNKAKSDLEEAKKWIRRANQKL

SEQ ID NO: 383 - protein, 91 aa, synthetic construct; identical to SEQ ID NO: 307

SEQ ID NO: 384 - protein, 372 aa, synthetic construct
FFGGVIGTIALGVATSAQITAAVALVEAKQARSDIEKLKEAIRDTNKAVQSVQSSVGNLIVAIKSVQDYVNKEIVPSIARLGCEAAGLQLGIALTQHYSELTNIFGDNIGSLQEKGIKLQAIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFILNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALDPIDISIELNKAKSDLEEAKEWIRRANQKL

SEQ ID NO: 385 - protein, 91 aa, synthetic construct; identical to SEQ ID NO: 307

SEQ ID NO: 386 - protein, 387 aa, synthetic construct
FVLGAIALGVATAAAVTAGVAIAKTIRLESEVTAIKNALKKTNEAVSTLGNGVRVLATAVRELKDFVSKNLTRAINKNKCDIDDLKMAVSFSQFNRRFLNVVRQFSDNAGITPAISLDLMTDAELARAVSNMPTSAGQIKLMLENRAMVRRKGFGILIGVYGSSVIYMVQLPIFGVIDTPCWIVKAAPSCSEKKGNYACLLREDQGWYCQNAGSTVYYPNEKDCETRGDHVFCDTAAGINVAEQSKECNINISTTNYPCKVSTGRHPISMVALSPLGALVACYKGVSCSIGSNRVGIIKQLNKGCSYITNQDADTVTIDNTVYQLSKVEGEQHVIKGRPVSSSFDPVKFPEDQFNVALDQVFENIENSQALVDCCNRILSSAEKGNT

SEQ ID NO: 387 - protein, 84 aa, synthetic construct; identical to SEQ ID NO: 24

SEQ ID NO: 388 - protein, 387 aa, synthetic construct
FVLGAIALGVATAAAVTAGIAIAKTIRLESEVNAIKGALKTTNEAVSTLGNGVRVLATAVRELKEFVSKNLTSAINKNKCDIADLKMAVSFSQFNRRFLNVVRQFSDNAGITPAISLDLMNDAELARAVSYMPTSAGQIKLMLENRAMVRRKGFGILIGVYGSSVIYMVQLPIFGVINTPCWIIKAAPSCSEKDGNYACLLREDQGWYCKNAGSTVYYPNEKDCETRGDHVFCDTAAGINVAEQSRECNINISTTNYPCKVSTGRHPISMVALSPLGALVACYKGVSCSIGSNQVGIIKQLPKGCSYITNQDADTVTIDNTVYQLSKVEGEQHVIKGRPVSSSFDPIRFPEDQFNVPLDQVFESIENSQALVDQSNKILNSAEKGNT

SEQ ID NO: 389 - protein, 84 aa, synthetic construct; identical to SEQ ID NO: 131

SEQ ID NO: 390 - DNA, 1617 bp, synthetic construct
  source 1..1617: mol_type = other DNA
atgagctggaaggtggtcatcatcttcagcctgctgatcacccctcagcacggcctgaaagagagctacctggaagaaagctgcagcaccatcaccgagggctacctgagcgtgctgagaaccggctggtacaccaacgtgttcaccctggaagtgggcgacgtggaaaacctgacctgtgccgatggacccagcctgatcaagaccgagctggacctgacaaagagcgccctgcgggaactgaaaaccgtgtctgcagatcagctggccagagaggaacagatcgagaaccccagacagagcagattcgtgctgggagctatcgccctgggagttgctacagctgctgctgtgacagctggcgtggccattgccaagaccatccggctggaaagcgaagtgaccgccatcaagaactgcctgaaaaagaccaacgaggccgtgtgcaccctcggcaatggcgttagagtgctggccacagccgtccgcgagctgaaggatttcgtgtccaagaacctgaccagggccatcaacaagaacaagtgcgacatcgacgacctgaagatggccgtgtccttcagccagttcaaccggcggttcctgaatgtcgtgcggcagttctctgacaacgccggcatcacaccagccatcagcctggatctgatgaccgatgccgaactggctagagccgtgtccaacatgcctacatctgccggccagatcaagctgatgctggaaaacagagccatggtccgacggaaaggcttcggcatcctgatcggcgtgtacggcagcagcgtgatctacatggtgcagctgcctatcttcggcgtgatcgacaccccttgctggatcgtgaaagccgctcctagctgcagcgagaagaagggcaattacgcctgcctgctgagagaggaccaaggctggtactgtcagaatgccggcagcaccgtgtactaccccaacgagaaggactgcgagacaagaggcgaccacgtgttctgtgataccgccgctggaatcaacgtggccgagcagagcaaagagtgcaacatcaacatcagcaccacaaactacccctgcaaggtgtccaccggcagacaccctatcagcatggtggctctgtctccactgggagccctggtggcttgttataagggcgtgtcctgtagcatcggcagcaacagagtgggcatcatcaagcagctgaacaagggctgctcctacatcaccaaccaggacgccgataccgtgaccatcgacaataccgtgtatcagctgagcaaggtggaaggcgaacagcacgtgatcaagggcagacctgtgtccagcagcttcgaccccgtgaagttccccgaggaccagttcaatgtggccctggaccaggtgttcgagaacatcgagaactctcaggctctggtggaccagtccaaccggattctgtctagcgccgagaagggaaacaccggcttcatcatcgtgatcatcctgattgccgtgctgggctccagcatgatcctggtgtccatctttatcatcatcaaaaagacgaagaagcccacaggcgcccctccagaactgtctggcgtgaccaacaatggcttcatccctcacagc

SEQ ID NO: 391 - RNA, 1617 nt, synthetic construct
  source 1..1617: mol_type = other RNA
atgagctggaaggtggtcatcatcttcagcctgctgatcacccctcagcacggcctgaaagagagctacctggaagaaagctgcagcaccatcaccgagggctacctgagcgtgctgagaaccggctggtacaccaacgtgttcaccctggaagtgggcgacgtggaaaacctgacctgtgccgatggacccagcctgatcaagaccgagctggacctgacaaagagcgccctgcgggaactgaaaaccgtgtctgcagatcagctggccagagaggaacagatcgagaaccccagacagagcagattcgtgctgggagctatcgccctgggagttgctacagctgctgctgtgacagctggcgtggccattgccaagaccatccggctggaaagcgaagtgaccgccatcaagaactgcctgaaaaagaccaacgaggccgtgtgcaccctcggcaatggcgttagagtgctggccacagccgtccgcgagctgaaggatttcgtgtccaagaacctgaccagggccatcaacaagaacaagtgcgacatcgacgacctgaagatggccgtgtccttcagccagttcaaccggcggttcctgaatgtcgtgcggcagttctctgacaacgccggcatcacaccagccatcagcctggatctgatgaccgatgccgaactggctagagccgtgtccaacatgcctacatctgccggccagatcaagctgatgctggaaaacagagccatggtccgacggaaaggcttcggcatcctgatcggcgtgtacggcagcagcgtgatctacatggtgcagctgcctatcttcggcgtgatcgacaccccttgctggatcgtgaaagccgctcctagctgcagcgagaagaagggcaattacgcctgcctgctgagagaggaccaaggctggtactgtcagaatgccggcagcaccgtgtactaccccaacgagaaggactgcgagacaagaggcgaccacgtgttctgtgataccgccgctggaatcaacgtggccgagcagagcaaagagtgcaacatcaacatcagcaccacaaactacccctgcaaggtgtccaccggcagacaccctatcagcatggtggctctgtctccactgggagccctggtggcttgttataagggcgtgtcctgtagcatcggcagcaacagagtgggcatcatcaagcagctgaacaagggctgctcctacatcaccaaccaggacgccgataccgtgaccatcgacaataccgtgtatcagctgagcaaggtggaaggcgaacagcacgtgatcaagggcagacctgtgtccagcagcttcgaccccgtgaagttccccgaggaccagttcaatgtggccctggaccaggtgttcgagaacatcgagaactctcaggctctggtggaccagtccaaccggattctgtctagcgccgagaagggaaacaccggcttcatcatcgtgatcatcctgattgccgtgctgggctccagcatgatcctggtgtccatctttatcatcatcaaaaagacgaagaagcccacaggcgcccctccagaactgtctggcgtgaccaacaatggcttcatccctcacagc

SEQ ID NO: 392 - DNA, 1617 bp, synthetic construct
  source 1..1617: mol_type = other DNA
atgagctggaaggtggtcatcatcttcagcctgctgatcacccctcagcacggcctgaaagagagctacctggaagaaagctgcagcaccatcaccgagggctacctgagcgtgctgagaaccggctggtacaccaacgtgttcaccctggaagtgggcgacgtggaaaacctgacctgtgccgatggacccagcctgatcaagaccgagctggacctgacaaagagcgccctgcgggaactgaaaaccgtgtctgcagatcagctggccagagaggaacagatcgagaaccccagacagagcagattcgtgctgggagctatcgccctgggagttgctacagctgctgctgtgacagctggcgtggccattgccaagaccatccggctggaaagcgaagtgaccgccatcaagaactgcctgaaaaagaccaacgaggccgtgtgcaccctcggcaatggcgttagagtgctggccacagccgtccgcgagctgaaggatttcgtgtccaagaacctgaccagggccatcaacaagaacaagtgcgacatcgacgacctgaagatggccgtgtccttcagccagttcaaccggcggttcctgaatgtcgtgcggcagttctctgacaacgccggcatcacaccagccatcagcctggatctgatgaccgatgccgaactggctagagccgtgtccaacatgcctacatctgccggccagatcaagctgatgctggaaaacagagccatggtccgacggaaaggcttcggcatcctgatcggcgtgtacggcagcagcgtgatctacatggtgcagctgcctatcttcggcgtgatcgacaccccttgctggatcgtgaaagccgctcctagctgcagcgagaagaagggcaattacgcctgcctgctgagagaggaccaaggctggtactgtcagaatgccggcagcaccgtgtactaccccaacgagaaggactgcgagacaagaggcgaccacgtgttctgtgataccgccgctggaatcaacgtggccgagcagagcaaagagtgcaacatcaacatcagcaccacaaactacccctgcaaggtgtccaccggcagacaccctatcagcatggtggctctgtctccactgggagccctggtggcttgttataagggcgtgtcctgtagcatcggcagcaacagagtgggcatcatcaagcagctgaacaagggctgctcctacatctgcaaccaggacgccgataccgtgaccatcgacaataccgtgtatcagctgagcaaggtggaaggcgaatgccacgtgatcaagggcagacctgtgtccagcagcttcgaccccgtgaagttccccgaggaccagttcaatgtggccctggaccaggtgttcgagaacatcgagaactctcaggctctggtggaccagtccaaccggattctgtctagcgccgagaagggaaacaccggcttcatcatcgtgatcatcctgattgccgtgctgggctccagcatgatcctggtgtccatctttatcatcatcaaaaagacgaagaagcccacaggcgcccctccagaactgtctggcgtgaccaacaatggcttcatccctcacagc

SEQ ID NO: 393 - RNA, 1617 nt, synthetic construct
  source 1..1617: mol_type = other RNA
atgagctggaaggtggtcatcatcttcagcctgctgatcacccctcagcacggcctgaaagagagctacctggaagaaagctgcagcaccatcaccgagggctacctgagcgtgctgagaaccggctggtacaccaacgtgttcaccctggaagtgggcgacgtggaaaacctgacctgtgccgatggacccagcctgatcaagaccgagctggacctgacaaagagcgccctgcgggaactgaaaaccgtgtctgcagatcagctggccagagaggaacagatcgagaaccccagacagagcagattcgtgctgggagctatcgccctgggagttgctacagctgctgctgtgacagctggcgtggccattgccaagaccatccggctggaaagcgaagtgaccgccatcaagaactgcctgaaaaagaccaacgaggccgtgtgcaccctcggcaatggcgttagagtgctggccacagccgtccgcgagctgaaggatttcgtgtccaagaacctgaccagggccatcaacaagaacaagtgcgacatcgacgacctgaagatggccgtgtccttcagccagttcaaccggcggttcctgaatgtcgtgcggcagttctctgacaacgccggcatcacaccagccatcagcctggatctgatgaccgatgccgaactggctagagccgtgtccaacatgcctacatctgccggccagatcaagctgatgctggaaaacagagccatggtccgacggaaaggcttcggcatcctgatcggcgtgtacggcagcagcgtgatctacatggtgcagctgcctatcttcggcgtgatcgacaccccttgctggatcgtgaaagccgctcctagctgcagcgagaagaagggcaattacgcctgcctgctgagagaggaccaaggctggtactgtcagaatgccggcagcaccgtgtactaccccaacgagaaggactgcgagacaagaggcgaccacgtgttctgtgataccgccgctggaatcaacgtggccgagcagagcaaagagtgcaacatcaacatcagcaccacaaactacccctgcaaggtgtccaccggcagacaccctatcagcatggtggctctgtctccactgggagccctggtggcttgttataagggcgtgtcctgtagcatcggcagcaacagagtgggcatcatcaagcagctgaacaagggctgctcctacatctgcaaccaggacgccgataccgtgaccatcgacaataccgtgtatcagctgagcaaggtggaaggcgaatgccacgtgatcaagggcagacctgtgtccagcagcttcgaccccgtgaagttccccgaggaccagttcaatgtggccctggaccaggtgttcgagaacatcgagaactctcaggctctggtggaccagtccaaccggattctgtctagcgccgagaagggaaacaccggcttcatcatcgtgatcatcctgattgccgtgctgggctccagcatgatcctggtgtccatctttatcatcatcaaaaagacgaagaagcccacaggcgcccctccagaactgtctggcgtgaccaacaatggcttcatccctcacagc

SEQ ID NO: 394 - DNA, 1617 bp, synthetic construct
  source 1..1617: mol_type = other DNA
atgagctggaaggtggtcatcatcttcagcctgctgatcacccctcagcacggcctgaaagagagctacctggaagaaagctgcagcaccatcaccgagggctacctgagcgtgctgagaaccggctggtacaccaacgtgttcaccctggaagtgggcgacgtggaaaacctgacctgtgccgatggacccagcctgatcaagaccgagctggacctgacaaagagcgccctgcgggaactgaaaaccgtgtctgcagatcagctggccagagaggaacagatcgagaaccccagacagagcagattcgtgctgggagctatcgccctgggagttgctacagctgctgctgtgacagctggcgtggccattgccaagaccatccggctggaaagcgaagtgaccgccatcaagaactgcctgaaaaagaccaacgaggccgtgtgcaccctcggcaatggcgttagagtgctggccacagccgtccgcgagctgaaggatttcgtgtccaagaacctgaccagggccatcaacaagaacaagtgcgacatcgacgacctgaagatggccgtgtccttcagccagttcaaccggcggttcctgaatgtcgtgcggcagttctctgacaacgccggcatcacaccagccatcagcctggatctgatgaccgatgccgaactggctagagccgtgtccaacatgcctacatctgccggccagatcaagctgatgctggaaaacagagccatggtccgacggaaaggcttcggcatcctgatcggcgtgtacggcagcagcgtgatctacatggtgcagctgcctatcttcggcgtgatcgacaccccttgctggatcgtgaaagccgctcctagctgcagcgagaagaagggcaattacgcctgcctgctgagagaggaccaaggctggtactgtcagaatgccggcagcaccgtgtactaccccaacgagaaggactgcgagacaagaggcgaccacgtgttctgtgataccgccgctggaatcaacgtggccgagcagagcaaagagtgcaacatcaacatcagcaccacaaactacccctgcaaggtgtccaccggcagacaccctatcagcatggtggctctgtctccactgggagccctggtggcttgttataagggcgtgtcctgtagcatcggcagcaacagagtgggcatcatcaagcagctgaacaagggctgctcctacatctgcaaccaggacgccgataccgtgaccatcgacaataccgtgtatcagctgagcaaggtggaaggcgaatgccacgtgatcaagggcagacctgtgtccagcagcttcgaccccgtgaagttccccgaggaccagttcaatgtgcccctggaccaggtgttcgagaacatcgagaactctcaggctctggtggaccagtccaaccggattctgtctagcgccgagaagggaaacaccggcttcatcatcgtgatcatcctgattgccgtgctgggctccagcatgatcctggtgtccatctttatcatcatcaaaaagacgaagaagcccacaggcgcccctccagaactgtctggcgtgaccaacaatggcttcatccctcacagc

SEQ ID NO: 395 - RNA, 1617 nt, synthetic construct
  source 1..1617: mol_type = other RNA
atgagctggaaggtggtcatcatcttcagcctgctgatcacccctcagcacggcctgaaagagagctacctggaagaaagctgcagcaccatcaccgagggctacctgagcgtgctgagaaccggctggtacaccaacgtgttcaccctggaagtgggcgacgtggaaaacctgacctgtgccgatggacccagcctgatcaagaccgagctggacctgacaaagagcgccctgcgggaactgaaaaccgtgtctgcagatcagctggccagagaggaacagatcgagaaccccagacagagcagattcgtgctgggagctatcgccctgggagttgctacagctgctgctgtgacagctggcgtggccattgccaagaccatccggctggaaagcgaagtgaccgccatcaagaactgcctgaaaaagaccaacgaggccgtgtgcaccctcggcaatggcgttagagtgctggccacagccgtccgcgagctgaaggatttcgtgtccaagaacctgaccagggccatcaacaagaacaagtgcgacatcgacgacctgaagatggccgtgtccttcagccagttcaaccggcggttcctgaatgtcgtgcggcagttctctgacaacgccggcatcacaccagccatcagcctggatctgatgaccgatgccgaactggctagagccgtgtccaacatgcctacatctgccggccagatcaagctgatgctggaaaacagagccatggtccgacggaaaggcttcggcatcctgatcggcgtgtacggcagcagcgtgatctacatggtgcagctgcctatcttcggcgtgatcgacaccccttgctggatcgtgaaagccgctcctagctgcagcgagaagaagggcaattacgcctgcctgctgagagaggaccaaggctggtactgtcagaatgccggcagcaccgtgtactaccccaacgagaaggactgcgagacaagaggcgaccacgtgttctgtgataccgccgctggaatcaacgtggccgagcagagcaaagagtgcaacatcaacatcagcaccacaaactacccctgcaaggtgtccaccggcagacaccctatcagcatggtggctctgtctccactgggagccctggtggcttgttataagggcgtgtcctgtagcatcggcagcaacagagtgggcatcatcaagcagctgaacaagggctgctcctacatctgcaaccaggacgccgataccgtgaccatcgacaataccgtgtatcagctgagcaaggtggaaggcgaatgccacgtgatcaagggcagacctgtgtccagcagcttcgaccccgtgaagttccccgaggaccagttcaatgtgcccctggaccaggtgttcgagaacatcgagaactctcaggctctggtggaccagtccaaccggattctgtctagcgccgagaagggaaacaccggcttcatcatcgtgatcatcctgattgccgtgctgggctccagcatgatcctggtgtccatctttatcatcatcaaaaagacgaagaagcccacaggcgcccctccagaactgtctggcgtgaccaacaatggcttcatccctcacagc

SEQ ID NO: 396 - DNA, 1617 bp, synthetic construct
  source 1..1617: mol_type = other DNA
atgagctggaaggtggtcatcatcttcagcctgctgatcacccctcagcacggcctgaaagagagctacctggaagaaagctgcagcaccatcaccgagggctacctgagcgtgctgagaaccggctggtacaccaacgtgttcaccctggaagtgggcgacgtggaaaacctgacctgtgccgatggacccagcctgatcaagaccgagctggacctgacaaagagcgccctgcgggaactgaaaaccgtgtctgcagatcagctggccagagaggaacagatcgagaaccccagacagagcagattcgtgctgggagctatcgccctgggagttgctacagctgctgctgtgacagctggcgtggccattgccaagaccatccggctggaaagcgaagtgaccgccatcaagaactgcctgaaaaagaccaacgaggccgtgtgcaccctcggcaatggcgttagagtgctggccacagccgtccgcgagctgaaggatttcgtgtccaagaacctgaccagggccatcaacaagaacaagtgcgacatcgacgacctgaagatggccgtgtccttcagccagttcaaccggcggttcctgaatgtcgtgcggcagttctctgacaacgccggcatcacaccagccatcagcctggatctgatgaccgatgccgaactggctagagccgtgtccaacatgcctacatctgccgcccagatcaagctgatgctggaaaacagagccatggtccgacggaaaggcttcggcatcctgatcggcgtgtacggcagcagcgtgatctacatggtgcagctgcctatcttcggcgtgatcgacaccccttgctggatcgtgaaagccgctcctagctgcagcgagaagaagggcaattacgcctgcctgctgagagaggaccaaggctggtactgtcagaatgccggcagcaccgtgtactaccccaacgagaaggactgcgagacaagaggcgaccacgtgttctgtgataccgccgctggaatcaacgtggccgagcagagcaaagagtgcaacatcaacatcagcaccacaaactacccctgcaaggtgtccaccggcagacaccctatcagcatggtggctctgtctccactgggagccctggtggcttgttataagggcgtgtcctgtagcatcggcagcaacagagtgggcatcatcaagcagctgaacaagggctgctcctacatctgcaaccaggacgccgataccgtgaccatcgacaataccgtgtatcagctgagcaaggtggaaggcgaatgccacgtgatcaagggcagacctgtgtccagcagcttcgaccccgtgaagttccccgaggaccagttcaatgtggccctggaccaggtgttcgagaacatcgagaactctcaggctctggtggaccagtccaaccggattctgtctagcgccgagaagggaaacaccggcttcatcatcgtgatcatcctgattgccgtgctgggctccagcatgatcctggtgtccatctttatcatcatcaaaaagacgaagaagcccacaggcgcccctccagaactgtctggcgtgaccaacaatggcttcatccctcacagc

SEQ ID NO: 397 - RNA, 1617 nt, synthetic construct
  source 1..1617: mol_type = other RNA
atgagctggaaggtggtcatcatcttcagcctgctgatcacccctcagcacggcctgaaagagagctacctggaagaaagctgcagcaccatcaccgagggctacctgagcgtgctgagaaccggctggtacaccaacgtgttcaccctggaagtgggcgacgtggaaaacctgacctgtgccgatggacccagcctgatcaagaccgagctggacctgacaaagagcgccctgcgggaactgaaaaccgtgtctgcagatcagctggccagagaggaacagatcgagaaccccagacagagcagattcgtgctgggagctatcgccctgggagttgctacagctgctgctgtgacagctggcgtggccattgccaagaccatccggctggaaagcgaagtgaccgccatcaagaactgcctgaaaaagaccaacgaggccgtgtgcaccctcggcaatggcgttagagtgctggccacagccgtccgcgagctgaaggatttcgtgtccaagaacctgaccagggccatcaacaagaacaagtgcgacatcgacgacctgaagatggccgtgtccttcagccagttcaaccggcggttcctgaatgtcgtgcggcagttctctgacaacgccggcatcacaccagccatcagcctggatctgatgaccgatgccgaactggctagagccgtgtccaacatgcctacatctgccgcccagatcaagctgatgctggaaaacagagccatggtccgacggaaaggcttcggcatcctgatcggcgtgtacggcagcagcgtgatctacatggtgcagctgcctatcttcggcgtgatcgacaccccttgctggatcgtgaaagccgctcctagctgcagcgagaagaagggcaattacgcctgcctgctgagagaggaccaaggctggtactgtcagaatgccggcagcaccgtgtactaccccaacgagaaggactgcgagacaagaggcgaccacgtgttctgtgataccgccgctggaatcaacgtggccgagcagagcaaagagtgcaacatcaacatcagcaccacaaactacccctgcaaggtgtccaccggcagacaccctatcagcatggtggctctgtctccactgggagccctggtggcttgttataagggcgtgtcctgtagcatcggcagcaacagagtgggcatcatcaagcagctgaacaagggctgctcctacatctgcaaccaggacgccgataccgtgaccatcgacaataccgtgtatcagctgagcaaggtggaaggcgaatgccacgtgatcaagggcagacctgtgtccagcagcttcgaccccgtgaagttccccgaggaccagttcaatgtggccctggaccaggtgttcgagaacatcgagaactctcaggctctggtggaccagtccaaccggattctgtctagcgccgagaagggaaacaccggcttcatcatcgtgatcatcctgattgccgtgctgggctccagcatgatcctggtgtccatctttatcatcatcaaaaagacgaagaagcccacaggcgcccctccagaactgtctggcgtgaccaacaatggcttcatccctcacagc

SEQ ID NO: 398 - DNA, 1617 bp, synthetic construct
  source 1..1617: mol_type = other DNA
atgagctggaaggtggtcatcatcttcagcctgctgatcacccctcagcacggcctgaaagagagctacctggaagaaagctgcagcaccatcaccgagggctacctgagcgtgctgagaaccggctggtacaccaacgtgttcatcctggaagtgggcgacgtggaaaacctgacctgtgccgatggacccagcctgatcaagaccgagctggacctgacaaagagcgccctgcgggaactgaaaaccgtgtctgcagatcagctggccagagaggaacagatcgagaaccccagacagagcagattcgtgctgggagctatcgccctgggagttgctacagctgctgctgtgacagctggcgtggccattgccaagaccatccggctggaaagcgaagtgaccgccatcaagaactgcctgaaaaagaccaacgaggccgtgtgcaccctcggcaatggcgttagagtgctggccacagccgtccgcgagctgaaggatttcgtgtccaagaacctgaccagggccatcaacaagaacaagtgcgacatcgacgacctgaagatggccgtgtccttcagccagttcaaccggcggttcctgaatgtcgtgcggcagttctctgacaacgccggcatcacaccagccatcagcctggatctgatgaccgatgccgaactggctagagccgtgtccaacatgcctacatctgccgcccagatcaagctgatgctggaaaacagagccatggtccgacggaaaggcttcggcatcctgatcggcgtgtacggcagcagcgtgatctacatggtgcagctgcctatcttcggcgtgatcgacaccccttgctggatcgtgaaagccgctcctagctgcagcgagaagaagggcaattacgcctgcctgctgagagaggaccaaggctggtactgtcagaatgccggcagcaccgtgtactaccccaacgagaaggactgcgagacaagaggcgaccacgtgttctgtgataccgccgctggaatcaacgtggccgagcagagcaaagagtgcaacatcaacatcagcaccacaaactacccctgcaaggtgtccatcggcagacaccctatcagcatggtggctctgtctccactgggagccctggtggcttgttataagggcgtgtcctgtagcatcggcagcaacagagtgggcatcatcaagcagctgaacaagggctgctcctacatctgcaaccaggacgccgataccgtgaccatcgacaataccgtgtatcagctgagcaaggtggaaggcgaatgccacgtgatcaagggcagacctgtgtccagcagcttcgaccccgtgaagttccccgaggaccagttcaatgtgcccctggaccaggtgttcgagaacatcgagaactctcaggctctggtggaccagtccaaccggattctgtctagcgccgagaagggaaacaccggcttcatcatcgtgatcatcctgattgccgtgctgggctccagcatgatcctggtgtccatctttatcatcatcaaaaagacgaagaagcccacaggcgcccctccagaactgtctggcgtgaccaacaatggcttcatccctcacagc

SEQ ID NO: 399 - RNA, 1617 nt, synthetic construct
  source 1..1617: mol_type = other RNA
atgagctggaaggtggtcatcatcttcagcctgctgatcacccctcagcacggcctgaaagagagctacctggaagaaagctgcagcaccatcaccgagggctacctgagcgtgctgagaaccggctggtacaccaacgtgttcatcctggaagtgggcgacgtggaaaacctgacctgtgccgatggacccagcctgatcaagaccgagctggacctgacaaagagcgccctgcgggaactgaaaaccgtgtctgcagatcagctggccagagaggaacagatcgagaaccccagacagagcagattcgtgctgggagctatcgccctgggagttgctacagctgctgctgtgacagctggcgtggccattgccaagaccatccggctggaaagcgaagtgaccgccatcaagaactgcctgaaaaagaccaacgaggccgtgtgcaccctcggcaatggcgttagagtgctggccacagccgtccgcgagctgaaggatttcgtgtccaagaacctgaccagggccatcaacaagaacaagtgcgacatcgacgacctgaagatggccgtgtccttcagccagttcaaccggcggttcctgaatgtcgtgcggcagttctctgacaacgccggcatcacaccagccatcagcctggatctgatgaccgatgccgaactggctagagccgtgtccaacatgcctacatctgccgcccagatcaagctgatgctggaaaacagagccatggtccgacggaaaggcttcggcatcctgatcggcgtgtacggcagcagcgtgatctacatggtgcagctgcctatcttcggcgtgatcgacaccccttgctggatcgtgaaagccgctcctagctgcagcgagaagaagggcaattacgcctgcctgctgagagaggaccaaggctggtactgtcagaatgccggcagcaccgtgtactaccccaacgagaaggactgcgagacaagaggcgaccacgtgttctgtgataccgccgctggaatcaacgtggccgagcagagcaaagagtgcaacatcaacatcagcaccacaaactacccctgcaaggtgtccatcggcagacaccctatcagcatggtggctctgtctccactgggagccctggtggcttgttataagggcgtgtcctgtagcatcggcagcaacagagtgggcatcatcaagcagctgaacaagggctgctcctacatctgcaaccaggacgccgataccgtgaccatcgacaataccgtgtatcagctgagcaaggtggaaggcgaatgccacgtgatcaagggcagacctgtgtccagcagcttcgaccccgtgaagttccccgaggaccagttcaatgtgcccctggaccaggtgttcgagaacatcgagaactctcaggctctggtggaccagtccaaccggattctgtctagcgccgagaagggaaacaccggcttcatcatcgtgatcatcctgattgccgtgctgggctccagcatgatcctggtgtccatctttatcatcatcaaaaagacgaagaagcccacaggcgcccctccagaactgtctggcgtgaccaacaatggcttcatccctcacagc

SEQ ID NO: 400 - DNA, 1665 bp, synthetic construct
  source 1..1665: mol_type = other DNA
atgcagagcagcgagatcctgctgctggtgtacagctctctgctgctgagcagctccctgtgtcagatccccgtggacaagctgagcaacgtgggcgtgatcatcaacgagggcaagctgctgaagatcgccggcagctacgagagccggtacatcgtgctgtctctggtgcccagcatcgacctgcaggatggctgtggcaccacacagatcatccagtacaagaacctgctgaaccggctgctgatccctctgaaggatgccctggatctgcaagagagcctgatcaccatcaccaacgacaccaccgtgaccaacgataaccctcagaccagaggctccggcgccgtgatcggaacaatcgccctgggagttgctaccgccgctcagatcacagccggaattgctctggccgaagccagagaggccagaaaggatatcgccctgatcaaggacagcatcgtgaaaacccacaacagcgtggaattcatccagcgcggcatcggcgagcagatcattgccctgaaaaccctgcaggacttcgtgaacgacgagatcagacccgccatcggagagctgagatgcgagacaacagccctgaagctgggcatcaagctgacccagcactacagcgaactggccaccgcctttagcagcaacctgggcacaatcggcgagaagtccctgacactgcaggccctgagcagcctgtacagcgccaatatcacagagatcctgtccaccatcaagaaggacaagagcgacatctacgacatcatctacaccgagcaagtgaagggcaccgtgatcgacgtggacctggaaaagtacatggtcaccctgctggtcaagatccccatcctgtctgagatccctggcgtgctgatctacagagccagcagcatcagctacaacatcgagggcgaagaatggcacgtggcaatccccaactacatcatcaacaaggccagcagcctcggcggagccgatgtgacaaattgcatcgagagcaagctggcctacatctgccccagagatcccacacagctgatccccgacaaccagcagaagtgcatcctgggcgacgtgtccaagtgtcctgtgaccaaagtgatcaacaacctggtgcctaagttcgccttcatcaacggcggcgtggtggccaactgtatcgccagcacatgcacatgcggcaccaacagaatccccgtgaaccaggacagatccaagggcgtgaccttcctgacctacaccaactgtggcctgatcggcatcaatggcatcgagctgtacgccaacaagcggggcagagataccacctggggcaaccagatcatcaaagtgggccctgccgtgtccatccggcctgtggatatctctctgaacctggccagcatcaccaactttctggaagaaatcaagaccgaactcatgaaggccagagccatcatctctgccgttggcggctggcacaacaaagagagcacccagatcattatcatcatcatcgtctgcatcctgatcatcattatctgcggcatcctgtactacctgtacagagtgcggagactgctcatcatgatcaacagcaccaacaatagccccatcaacgcctacacactggaaagccggatgaagaacccctacatgggcaaccacagcaac

SEQ ID NO: 401 - RNA, 1665 nt, synthetic construct
  source 1..1665: mol_type = other RNA
atgcagagcagcgagatcctgctgctggtgtacagctctctgctgctgagcagctccctgtgtcagatccccgtggacaagctgagcaacgtgggcgtgatcatcaacgagggcaagctgctgaagatcgccggcagctacgagagccggtacatcgtgctgtctctggtgcccagcatcgacctgcaggatggctgtggcaccacacagatcatccagtacaagaacctgctgaaccggctgctgatccctctgaaggatgccctggatctgcaagagagcctgatcaccatcaccaacgacaccaccgtgaccaacgataaccctcagaccagaggctccggcgccgtgatcggaacaatcgccctgggagttgctaccgccgctcagatcacagccggaattgctctggccgaagccagagaggccagaaaggatatcgccctgatcaaggacagcatcgtgaaaacccacaacagcgtggaattcatccagcgcggcatcggcgagcagatcattgccctgaaaaccctgcaggacttcgtgaacgacgagatcagacccgccatcggagagctgagatgcgagacaacagccctgaagctgggcatcaagctgacccagcactacagcgaactggccaccgcctttagcagcaacctgggcacaatcggcgagaagtccctgacactgcaggccctgagcagcctgtacagcgccaatatcacagagatcctgtccaccatcaagaaggacaagagcgacatctacgacatcatctacaccgagcaagtgaagggcaccgtgatcgacgtggacctggaaaagtacatggtcaccctgctggtcaagatccccatcctgtctgagatccctggcgtgctgatctacagagccagcagcatcagctacaacatcgagggcgaagaatggcacgtggcaatccccaactacatcatcaacaaggccagcagcctcggcggagccgatgtgacaaattgcatcgagagcaagctggcctacatctgccccagagatcccacacagctgatccccgacaaccagcagaagtgcatcctgggcgacgtgtccaagtgtcctgtgaccaaagtgatcaacaacctggtgcctaagttcgccttcatcaacggcggcgtggtggccaactgtatcgccagcacatgcacatgcggcaccaacagaatccccgtgaaccaggacagatccaagggcgtgaccttcctgacctacaccaactgtggcctgatcggcatcaatggcatcgagctgtacgccaacaagcggggcagagataccacctggggcaaccagatcatcaaagtgggccctgccgtgtccatccggcctgtggatatctctctgaacctggccagcatcaccaactttctggaagaaatcaagaccgaactcatgaaggccagagccatcatctctgccgttggcggctggcacaacaaagagagcacccagatcattatcatcatcatcgtctgcatcctgatcatcattatctgcggcatcctgtactacctgtacagagtgcggagactgctcatcatgatcaacagcaccaacaatagccccatcaacgcctacacactggaaagccggatgaagaacccctacatgggcaaccacagcaac

SEQ ID NO: 402 - DNA, 1665 bp, synthetic construct
  source 1..1665: mol_type = other DNA
atgcagagcagcgagatcctgctgctggtgtacagctctctgctgctgagcagctccctgtgtcagatccccgtggacaagctgagcaacgtgggcgtgatcatcaacgagggcaagctgctgaagatcgccggcagctacgagagccggtacatcgtgctgtctctggtgcccagcatcgacctgcaggatggctgtggcaccacacagatcatccagtacaagaacctgctgaaccggctgctgatccctctgaaggatgccctggatctgtgcgagagcctgatcaccatcaccaacgacaccaccgtgaccaacgataaccctcagaccagaggcagcggcgccgtgatcggaacaatcgccctgggagttgctaccgccgctcagatcacagcctgcattgctctggccgaagccagagaggccagaaaggatatcgccctgatcaaggacagcatcgtgaaaacccacaacagcgtggaattcatccagcgcggcatcggcgagcagatcattgccctgaaaaccctgcaggacttcgtgaacgacgagatcagacccgccatcggagagctgagatgcgagacaacagccctgaagctgggcatcaagctgacccagcactacagcgaactggccaccgcctttagcagcaacctgggcacaatcggcgagaagtccctgacactgcaggccctgagcagcctgtacagcgccaatatcacagagatcctgtccaccatcaagaaggacaagagcgacatctacgacatcatctacaccgagcaagtgaagggcaccgtgatcgacgtggacctggaaaagtacatggtcaccctgctggtcaagatccccatcctgtctgagatccctggcgtgctgatctacagagccagcagcatcagctacaacatcgagggcgaagaatggcacgtggcaatccccaactacatcatcaacaaggccagcagcctcggcggagccgatgtgacaaattgcatcgagagcaagctggcctacatctgccccagagatcccacacagctgatccccgacaaccagcagaagtgcatcctgggcgacgtgtccaagtgtcctgtgaccaaagtgatcaacaacctggtgcctaagttcgccttcatcaacggcggcgtggtggccaactgtatcgccagcacatgcacatgcggcaccaacagaatccccgtgaaccaggacagatccaagggcgtgaccttcctgacctacaccaactgtggcctgatcggcatcaatggcatcgagctgtacgccaacaagcggggcagagataccacctggggcaaccagatcatcaaagtgggccctgccgtgtccatccggcctgtggatatctctctgaacctggccagcctgaccaactttctggaagaactgaagaccgaactcatgaagctgagagccatcatctctgccgttggcggctggcacaacaaagagagcacccagatcattatcatcatcatcgtctgcatcctgatcatcattatctgcggcatcctgtactacctgtacagagtgcggagactgctcatcatgatcaacagcaccaacaatagccccatcaacgcctacacactggaaagccggatgaagaacccctacatgggcaaccacagcaac

SEQ ID NO: 403 - RNA, 1665 nt, synthetic construct
  source 1..1665: mol_type = other RNA
atgcagagcagcgagatcctgctgctggtgtacagctctctgctgctgagcagctccctgtgtcagatccccgtggacaagctgagcaacgtgggcgtgatcatcaacgagggcaagctgctgaagatcgccggcagctacgagagccggtacatcgtgctgtctctggtgcccagcatcgacctgcaggatggctgtggcaccacacagatcatccagtacaagaacctgctgaaccggctgctgatccctctgaaggatgccctggatctgtgcgagagcctgatcaccatcaccaacgacaccaccgtgaccaacgataaccctcagaccagaggcagcggcgccgtgatcggaacaatcgccctgggagttgctaccgccgctcagatcacagcctgcattgctctggccgaagccagagaggccagaaaggatatcgccctgatcaaggacagcatcgtgaaaacccacaacagcgtggaattcatccagcgcggcatcggcgagcagatcattgccctgaaaaccctgcaggacttcgtgaacgacgagatcagacccgccatcggagagctgagatgcgagacaacagccctgaagctgggcatcaagctgacccagcactacagcgaactggccaccgcctttagcagcaacctgggcacaatcggcgagaagtccctgacactgcaggccctgagcagcctgtacagcgccaatatcacagagatcctgtccaccatcaagaaggacaagagcgacatctacgacatcatctacaccgagcaagtgaagggcaccgtgatcgacgtggacctggaaaagtacatggtcaccctgctggtcaagatccccatcctgtctgagatccctggcgtgctgatctacagagccagcagcatcagctacaacatcgagggcgaagaatggcacgtggcaatccccaactacatcatcaacaaggccagcagcctcggcggagccgatgtgacaaattgcatcgagagcaagctggcctacatctgccccagagatcccacacagctgatccccgacaaccagcagaagtgcatcctgggcgacgtgtccaagtgtcctgtgaccaaagtgatcaacaacctggtgcctaagttcgccttcatcaacggcggcgtggtggccaactgtatcgccagcacatgcacatgcggcaccaacagaatccccgtgaaccaggacagatccaagggcgtgaccttcctgacctacaccaactgtggcctgatcggcatcaatggcatcgagctgtacgccaacaagcggggcagagataccacctggggcaaccagatcatcaaagtgggccctgccgtgtccatccggcctgtggatatctctctgaacctggccagcctgaccaactttctggaagaactgaagaccgaactcatgaagctgagagccatcatctctgccgttggcggctggcacaacaaagagagcacccagatcattatcatcatcatcgtctgcatcctgatcatcattatctgcggcatcctgtactacctgtacagagtgcggagactgctcatcatgatcaacagcaccaacaatagccccatcaacgcctacacactggaaagccggatgaagaacccctacatgggcaaccacagcaac

SEQ ID NO: 404 - DNA, 1665 bp, synthetic construct
  source 1..1665: mol_type = other DNA
atgcagagcagcgagatcctgctgctggtgtacagctctctgctgctgagcagctccctgtgtcagatccccgtggacaagctgagcaacgtgggcgtgatcatcaacgagggcaagctgctgaagatcgccggcagctacgagagccggtacatcgtgctgtctctggtgcccagcatcgacctgcaggatggctgtggcaccacacagatcatccagtacaagaacctgctgaaccggctgctgatccctctgaaggatgccctggatctgcaagagagcctgatcaccatcaccaacgacaccaccgtgaccaacgataaccctcagaccagaggcagcggcgccgtgatcggaacaatcgccctgggagttgctaccgccgctcagatcacagccgccattgctctggccgaagccagagaggccagaaaggatatcgccctgatcaaggacagcatcgtgaaaacccacaacagcgtggaattcatccagcgcggcatcggcgagcagatcattgccctgaaaaccctgcaggacttcgtgaacgacgagatcagacccgccatcggagagctgagatgcgagacaacagccctgaagctgggcatcaagctgacccagcactacagcgaactggccaccgcctttagcagcaacctgggcacaatcggcgagaagtccctgacactgcaggccctgagcagcctgtacagcgccaatatcacagagatcctgtccaccatcaagaaggacaagagcgacatctacgacatcatctacaccgagcaagtgaagggcaccgtgatcgacgtggacctggaaaagtacatggtcaccctgctggtcaagatccccatcctgtctgagatccctggcgtgctgatctacagagccagcagcatcagctacaacatcgagggcgaagaatggcacgtggcaatccccaactacatcatcaacaaggccagcagcctcggcggagccgatgtgacaaattgcatcgagagcaagctggcctacatctgccccagagatcccacacagctgatccccgacaaccagcagaagtgcatcctgggcgacgtgtccaagtgtcctgtgaccaaagtgatcaacaacctggtgcctaagttcgccttcatcaacggcggcgtggtggccaactgtatcgccagcacatgcacatgcggcaccaacagaatccccgtgaaccaggacagatccaagggcgtgaccttcctgacctacaccaactgtggcctgatcggcatcaatggcatcgagctgtacgccaacaagcggggcagagataccacctggggcaaccagatcatcaaagtgggccctgccgtgtccatccggcctgtggatatctctctgaacctggccagcctgaccaactttctggaagaactgaagaccgaactcatgaagctgagagccatcatctctgccgttggcggctggcacaacaaagagagcacccagatcattatcatcatcatcgtctgcatcctgatcatcattatctgcggcatcctgtactacctgtacagagtgcggagactgctcatcatgatcaacagcaccaacaatagccccatcaacgcctacacactggaaagccggatgaagaacccctacatgggcaaccacagcaac

SEQ ID NO: 405 - RNA, 1665 nt, synthetic construct
  source 1..1665: mol_type = other RNA
atgcagagcagcgagatcctgctgctggtgtacagctctctgctgctgagcagctccctgtgtcagatccccgtggacaagctgagcaacgtgggcgtgatcatcaacgagggcaagctgctgaagatcgccggcagctacgagagccggtacatcgtgctgtctctggtgcccagcatcgacctgcaggatggctgtggcaccacacagatcatccagtacaagaacctgctgaaccggctgctgatccctctgaaggatgccctggatctgcaagagagcctgatcaccatcaccaacgacaccaccgtgaccaacgataaccctcagaccagaggcagcggcgccgtgatcggaacaatcgccctgggagttgctaccgccgctcagatcacagccgccattgctctggccgaagccagagaggccagaaaggatatcgccctgatcaaggacagcatcgtgaaaacccacaacagcgtggaattcatccagcgcggcatcggcgagcagatcattgccctgaaaaccctgcaggacttcgtgaacgacgagatcagacccgccatcggagagctgagatgcgagacaacagccctgaagctgggcatcaagctgacccagcactacagcgaactggccaccgcctttagcagcaacctgggcacaatcggcgagaagtccctgacactgcaggccctgagcagcctgtacagcgccaatatcacagagatcctgtccaccatcaagaaggacaagagcgacatctacgacatcatctacaccgagcaagtgaagggcaccgtgatcgacgtggacctggaaaagtacatggtcaccctgctggtcaagatccccatcctgtctgagatccctggcgtgctgatctacagagccagcagcatcagctacaacatcgagggcgaagaatggcacgtggcaatccccaactacatcatcaacaaggccagcagcctcggcggagccgatgtgacaaattgcatcgagagcaagctggcctacatctgccccagagatcccacacagctgatccccgacaaccagcagaagtgcatcctgggcgacgtgtccaagtgtcctgtgaccaaagtgatcaacaacctggtgcctaagttcgccttcatcaacggcggcgtggtggccaactgtatcgccagcacatgcacatgcggcaccaacagaatccccgtgaaccaggacagatccaagggcgtgaccttcctgacctacaccaactgtggcctgatcggcatcaatggcatcgagctgtacgccaacaagcggggcagagataccacctggggcaaccagatcatcaaagtgggccctgccgtgtccatccggcctgtggatatctctctgaacctggccagcctgaccaactttctggaagaactgaagaccgaactcatgaagctgagagccatcatctctgccgttggcggctggcacaacaaagagagcacccagatcattatcatcatcatcgtctgcatcctgatcatcattatctgcggcatcctgtactacctgtacagagtgcggagactgctcatcatgatcaacagcaccaacaatagccccatcaacgcctacacactggaaagccggatgaagaacccctacatgggcaaccacagcaac

SEQ ID NO: 406 - DNA, 1665 bp, synthetic construct
  source 1..1665: mol_type = other DNA
atgcagagcagcgagatcctgctgctggtgtacagctctctgctgctgagcagctccctgtgtcagatccccgtggacaagctgagcaacgtgggcgtgatcatcaacgagggcaagctgctgaagatcgccggcagctacgagagccggtacatcgtgctgtctctggtgcccagcatcgacctgcaggatggctgtggcaccacacagatcatccagtacaagaacctgctgaaccggctgctgatccctctgaaggatgccctggatctgcaagagagcctgatcaccatcaccaacgacaccaccgtgaccaacgataaccctcagaccagattcttcggcgccgtgatcggaacaatcgccctgggagttgctaccgccgctcagatcacagccgccattgctctggccgaagccagagaggccagaaaggatatcgccctgatcaaggacagcatcgtgaaaacccacaacagcgtggaattcatccagcgcggcatcggcgagcagatcattgccctgaaaaccctgcaggacttcgtgaacgacgagatcagacccgccatcggagagctgagatgcgagacaacagccctgaagctgggcatcaagctgacccagcactacagcgaactggccaccgcctttagcagcaacctgggcacaatcggcgagaagtccctgacactgcaggccctgagcagcctgtacagcgccaatatcacagagatcctgtccaccatcaagaaggacaagagcgacatctacgacatcatctacaccgagcaagtgaagggcaccgtgatcgacgtggacctggaaaagtacatggtcaccctgctggtcaagatccccatcctgtctgagatccctggcgtgctgatctacagagccagcagcatcagctacaacatcgagggcgaagaatggcacgtggcaatccccaactacatcatcaacaaggccagcagcctcggcggagccgatgtgacaaattgcatcgagagcaagctggcctacatctgccccagagatcccacacagctgatccccgacaaccagcagaagtgcatcctgggcgacgtgtccaagtgtcctgtgaccaaagtgatcaacaacctggtgcctaagttcgccttcatcaacggcggcgtggtggccaactgtatcgccagcacatgcacatgcggcaccaacagaatccccgtgaaccaggacagatccaagggcgtgaccttcctgacctacaccaactgtggcctgatcggcatcaatggcatcgagctgtacgccaacaagcggggcagagataccacctggggcaaccagatcatcaaagtgggccctgccgtgtccatccggcctgtggatatctctctgaacctggccagcctgaccaactttctggaagaactgaagaccgaactcatgaagctgagagccatcatctctgccgttggcggctggcacaacaaagagagcacccagatcattatcatcatcatcgtctgcatcctgatcatcattatctgcggcatcctgtactacctgtacagagtgcggagactgctcatcatgatcaacagcaccaacaatagccccatcaacgcctacacactggaaagccggatgaagaacccctacatgggcaaccacagcaac

SEQ ID NO: 407 - RNA, 1665 nt, synthetic construct
  source 1..1665: mol_type = other RNA
atgcagagcagcgagatcctgctgctggtgtacagctctctgctgctgagcagctccctgtgtcagatccccgtggacaagctgagcaacgtgggcgtgatcatcaacgagggcaagctgctgaagatcgccggcagctacgagagccggtacatcgtgctgtctctggtgcccagcatcgacctgcaggatggctgtggcaccacacagatcatccagtacaagaacctgctgaaccggctgctgatccctctgaaggatgccctggatctgcaagagagcctgatcaccatcaccaacgacaccaccgtgaccaacgataaccctcagaccagattcttcggcgccgtgatcggaacaatcgccctgggagttgctaccgccgctcagatcacagccgccattgctctggccgaagccagagaggccagaaaggatatcgccctgatcaaggacagcatcgtgaaaacccacaacagcgtggaattcatccagcgcggcatcggcgagcagatcattgccctgaaaaccctgcaggacttcgtgaacgacgagatcagacccgccatcggagagctgagatgcgagacaacagccctgaagctgggcatcaagctgacccagcactacagcgaactggccaccgcctttagcagcaacctgggcacaatcggcgagaagtccctgacactgcaggccctgagcagcctgtacagcgccaatatcacagagatcctgtccaccatcaagaaggacaagagcgacatctacgacatcatctacaccgagcaagtgaagggcaccgtgatcgacgtggacctggaaaagtacatggtcaccctgctggtcaagatccccatcctgtctgagatccctggcgtgctgatctacagagccagcagcatcagctacaacatcgagggcgaagaatggcacgtggcaatccccaactacatcatcaacaaggccagcagcctcggcggagccgatgtgacaaattgcatcgagagcaagctggcctacatctgccccagagatcccacacagctgatccccgacaaccagcagaagtgcatcctgggcgacgtgtccaagtgtcctgtgaccaaagtgatcaacaacctggtgcctaagttcgccttcatcaacggcggcgtggtggccaactgtatcgccagcacatgcacatgcggcaccaacagaatccccgtgaaccaggacagatccaagggcgtgaccttcctgacctacaccaactgtggcctgatcggcatcaatggcatcgagctgtacgccaacaagcggggcagagataccacctggggcaaccagatcatcaaagtgggccctgccgtgtccatccggcctgtggatatctctctgaacctggccagcctgaccaactttctggaagaactgaagaccgaactcatgaagctgagagccatcatctctgccgttggcggctggcacaacaaagagagcacccagatcattatcatcatcatcgtctgcatcctgatcatcattatctgcggcatcctgtactacctgtacagagtgcggagactgctcatcatgatcaacagcaccaacaatagccccatcaacgcctacacactggaaagccggatgaagaacccctacatgggcaaccacagcaac

SEQ ID NO: 408 - DNA, 1665 bp, synthetic construct
  source 1..1665: mol_type = other DNA
atgcagagcagcgagatcctgctgctggtgtacagctctctgctgctgagcagctccctgtgtcagatccccgtggacaagctgagcaacgtgggcgtgatcatcaacgagggcaagctgctgaagatcgccggcagctacgagagccggtacatcgtgctgtctctggtgcccagcatcgacctgcaggatggctgtggcaccacacagatcatccagtacaagaacctgctgaaccggctgctgatccctctgaaggatgccctggatctgcaagagagcctgatcaccatcaccaacgacaccaccgtgaccaacgataaccctcagaccagaggcagcggcgccgtgatcggaacaatcgccctgggagttgctaccgccgctcagatcacagccgccattgctctggccgaagccagagaggccagaaaggatatcgccctgatcaaggacagcatcgtgaaaacccacaacagcgtggaattcatccagcgcggcatcggcgagcagatcattgccctgaaaaccctgcaggacttcgtgaacgacgagatcagacccgccatcggagagctgagatgcgagacaacagccctgaagctgggcatcaagctgacccagcactacagcgaactggccaccgcctttagcagcaacctgggcacaatcggcgagaagtccctgacactgcaggccctgagcagcctgtacagcgccaatatcacagagatcctgtccaccatcaagaaggacaagagcgacatctacgacatcatctacaccgagcaagtgaagggcaccgtgatcgacgtggacctggaaaagtacatggtcaccctgctggtcaagatccccatcctgtctgagatccctggcgtgctgatctacagagccagcagcatcagctacaacatcgagggcgaagaatggcacgtggcaatccccaactacatcatcaacaaggccagcagcctcggcggagccgatgtgacaaattgcatcgagagcaagctggcctacatctgccccagagatcccacacagctgatccccgacaaccagcagaagtgcatcctgggcgacgtgtccaagtgtcctgtgaccaaagtgatcaacaacctggtgcctaagttcgccttcatcaacggcggcgtggtggccaactgtatcgccagcacatgcacatgcggcaccaacagaatccccgtgaaccaggacagatccaagggcgtgaccttcctgacctacaccaactgtggcctgatcggcatcaatggcatcgagctgtacgccaacaagcggggcagagataccacctggggcaaccagatcatcaaagtgggccctgccgtgtccatccggcctgtggatatctctctgaacctggccagcctgaccaactttctggaagaactgaagaccgaactcatgaaggccagagccatcatctctgccgttggcggctggcacaacaaagagagcacccagatcattatcatcatcatcgtctgcatcctgatcatcattatctgcggcatcctgtactacctgtacagagtgcggagactgctcatcatgatcaacagcaccaacaatagccccatcaacgcctacacactggaaagccggatgaagaacccctacatgggcaaccacagcaac

SEQ ID NO: 409 - RNA, 1665 nt, synthetic construct
  source 1..1665: mol_type = other RNA
atgcagagcagcgagatcctgctgctggtgtacagctctctgctgctgagcagctccctgtgtcagatccccgtggacaagctgagcaacgtgggcgtgatcatcaacgagggcaagctgctgaagatcgccggcagctacgagagccggtacatcgtgctgtctctggtgcccagcatcgacctgcaggatggctgtggcaccacacagatcatccagtacaagaacctgctgaaccggctgctgatccctctgaaggatgccctggatctgcaagagagcctgatcaccatcaccaacgacaccaccgtgaccaacgataaccctcagaccagaggcagcggcgccgtgatcggaacaatcgccctgggagttgctaccgccgctcagatcacagccgccattgctctggccgaagccagagaggccagaaaggatatcgccctgatcaaggacagcatcgtgaaaacccacaacagcgtggaattcatccagcgcggcatcggcgagcagatcattgccctgaaaaccctgcaggacttcgtgaacgacgagatcagacccgccatcggagagctgagatgcgagacaacagccctgaagctgggcatcaagctgacccagcactacagcgaactggccaccgcctttagcagcaacctgggcacaatcggcgagaagtccctgacactgcaggccctgagcagcctgtacagcgccaatatcacagagatcctgtccaccatcaagaaggacaagagcgacatctacgacatcatctacaccgagcaagtgaagggcaccgtgatcgacgtggacctggaaaagtacatggtcaccctgctggtcaagatccccatcctgtctgagatccctggcgtgctgatctacagagccagcagcatcagctacaacatcgagggcgaagaatggcacgtggcaatccccaactacatcatcaacaaggccagcagcctcggcggagccgatgtgacaaattgcatcgagagcaagctggcctacatctgccccagagatcccacacagctgatccccgacaaccagcagaagtgcatcctgggcgacgtgtccaagtgtcctgtgaccaaagtgatcaacaacctggtgcctaagttcgccttcatcaacggcggcgtggtggccaactgtatcgccagcacatgcacatgcggcaccaacagaatccccgtgaaccaggacagatccaagggcgtgaccttcctgacctacaccaactgtggcctgatcggcatcaatggcatcgagctgtacgccaacaagcggggcagagataccacctggggcaaccagatcatcaaagtgggccctgccgtgtccatccggcctgtggatatctctctgaacctggccagcctgaccaactttctggaagaactgaagaccgaactcatgaaggccagagccatcatctctgccgttggcggctggcacaacaaagagagcacccagatcattatcatcatcatcgtctgcatcctgatcatcattatctgcggcatcctgtactacctgtacagagtgcggagactgctcatcatgatcaacagcaccaacaatagccccatcaacgcctacacactggaaagccggatgaagaacccctacatgggcaaccacagcaac

SEQ ID NO: 410 - DNA, 1617 bp, synthetic construct
  source 1..1617: mol_type = other DNA
atgctgatctccatcctgctgatcatcaccacaatgatcatggccagccactgccagatcgacatcaccaagctgcagcacgtgggcgtgctggtcaatagccctaagggcatgaagatcagccagaacttcgagacacgctacctgatcctgtctctgatccccaagatcgaggacagcaacagctgcggcgaccagcagatcaagcagtacaagcggctgctggacagactgatcatccctctgtacgacggcctgcggctgcagaaagatgtgatcgtgaccaatcaagagagcaacgagaacacagaccccagaaccgagagattcttcggcggcgtgatcggcacaatcgccctgggagttgctacaagcgcccagattacagccgccgtggctctggtggaagccaagcaggccagaagcgacatcgagaagctgaaagaggccatccgggacaccaacaaggccgtgcagtgcgtgcaaagcagcgtgggcaatctgatctgcgccattaagagcgtgcaggactacgtgaacaaagagatcgtcccctctatcgccagactgggatgtgaagctgccggactgcagctgggaattgccctgacacagcactacagcgagctgaccaacatcttcggcgacaacatcggcagcctgcaagagaagggcattaagctgcagggaatcgccagcctgtaccgcaccaacatcaccgagatcttcaccaccagcaccgtggataagtacgacatctacgacctgctgttcaccgagagcatcaaagtgcgcgtgatcgacgtggacctgaacgactacagcatcaccctgcaagtgcggctgcctctgctgaccagactgctgaacacccagatctacaaggtggacagcatctcctacaacatccagaaccgcgagtggtacatcccactgcctagccacattatgaccaagggcgcctttctcggcggagccgatgtgaaagagtgcatcgaggccttcagcagctacatctgccctagcgatcctggcttcgtgctgaaccacgagatggaaagctgcctgagcggcaacatctctcagtgccctagaaccaccgtgacctccgacatcgtgcccagatacgcctttgtgaatggcggcgtggtggccaactgcatcaccaccacctgtacctgcaacggcatcggcaaccggatcaaccagcctccagatcagggcgtgaagattatcacccacaaagagtgtaacaccatcggcatcaacggcatgctgttcaataccaacaaagagggcaccctggccttctacacccctgacgatatcaccctgaacaacagcgtggccctggatcctatcgacatctccatcgagctgaacaaggccaagagcgacctggaagaagccaaagagtggatccggcgggccaaccagaagctggatagcatcggaagctggcaccagagcagcaccaccatcatcgtgatcctgattatgatgattatcctgttcatcatcaacattaccatcatcacgatcgccatcaagtactaccggatccagaaacggaaccgcgtggaccagaatgacaagccctacgtgctgacaaacaag

SEQ ID NO: 411 - RNA, 1617 nt, synthetic construct
  source 1..1617: mol_type = other RNA
atgctgatctccatcctgctgatcatcaccacaatgatcatggccagccactgccagatcgacatcaccaagctgcagcacgtgggcgtgctggtcaatagccctaagggcatgaagatcagccagaacttcgagacacgctacctgatcctgtctctgatccccaagatcgaggacagcaacagctgcggcgaccagcagatcaagcagtacaagcggctgctggacagactgatcatccctctgtacgacggcctgcggctgcagaaagatgtgatcgtgaccaatcaagagagcaacgagaacacagaccccagaaccgagagattcttcggcggcgtgatcggcacaatcgccctgggagttgctacaagcgcccagattacagccgccgtggctctggtggaagccaagcaggccagaagcgacatcgagaagctgaaagaggccatccgggacaccaacaaggccgtgcagtgcgtgcaaagcagcgtgggcaatctgatctgcgccattaagagcgtgcaggactacgtgaacaaagagatcgtcccctctatcgccagactgggatgtgaagctgccggactgcagctgggaattgccctgacacagcactacagcgagctgaccaacatcttcggcgacaacatcggcagcctgcaagagaagggcattaagctgcagggaatcgccagcctgtaccgcaccaacatcaccgagatcttcaccaccagcaccgtggataagtacgacatctacgacctgctgttcaccgagagcatcaaagtgcgcgtgatcgacgtggacctgaacgactacagcatcaccctgcaagtgcggctgcctctgctgaccagactgctgaacacccagatctacaaggtggacagcatctcctacaacatccagaaccgcgagtggtacatcccactgcctagccacattatgaccaagggcgcctttctcggcggagccgatgtgaaagagtgcatcgaggccttcagcagctacatctgccctagcgatcctggcttcgtgctgaaccacgagatggaaagctgcctgagcggcaacatctctcagtgccctagaaccaccgtgacctccgacatcgtgcccagatacgcctttgtgaatggcggcgtggtggccaactgcatcaccaccacctgtacctgcaacggcatcggcaaccggatcaaccagcctccagatcagggcgtgaagattatcacccacaaagagtgtaacaccatcggcatcaacggcatgctgttcaataccaacaaagagggcaccctggccttctacacccctgacgatatcaccctgaacaacagcgtggccctggatcctatcgacatctccatcgagctgaacaaggccaagagcgacctggaagaagccaaagagtggatccggcgggccaaccagaagctggatagcatcggaagctggcaccagagcagcaccaccatcatcgtgatcctgattatgatgattatcctgttcatcatcaacattaccatcatcacgatcgccatcaagtactaccggatccagaaacggaaccgcgtggaccagaatgacaagccctacgtgctgacaaacaag

SEQ ID NO: 412 - DNA, 1617 bp, synthetic construct
  source 1..1617: mol_type = other DNA
atgctgatctccatcctgctgatcatcaccacaatgatcatggccagccactgccagatcgacatcaccaagctgcagcacgtgggcgtgctggtcaatagccctaagggcatgaagatcagccagaacttcgagacacgctacctgatcctgtctctgatccccaagatcgaggacagcaacagctgcggcgaccagcagatcaagcagtacaagcggctgctggacagactgatcatccctctgtacgacggcctgcggctgcagaaagatgtgatcgtgaccaatcaagagagcaacgagaacacagaccccagaaccgagagattcttcggcggcgtgatcggcacaatcgccctgggagttgctacaagcgcccagattacagccgccgtggctctggtggaagccaagcaggccagaagcgacatcgagaagctgaaagaggccatccgggacaccaacaaggccgtgcagtctgtgcaaagcagcgtgggcaatctgatcgtggccattaagagcgtgcaggactacgtgaacaaagagatcgtcccctctatcgccagactgggatgtgaagctgccggactgcagctgggaattgccctgacacagcactacagcgagctgaccaacatcttcggcgacaacatcggcagcctgcaagagaagggcattaagctgcaggccatcgccagcctgtaccgcaccaacatcaccgagatcttcaccaccagcaccgtggataagtacgacatctacgacctgctgttcaccgagagcatcaaagtgcgcgtgatcgacgtggacctgaacgactacagcatcaccctgcaagtgcggctgcctctgctgaccagactgctgaacacccagatctacaaggtggacagcatctcctacaacatccagaaccgcgagtggtacatcccactgcctagccacattatgaccaagggcgcctttctcggcggagccgatgtgaaagagtgcatcgaggccttcagcagctacatctgccctagcgatcctggcttcgtgctgaaccacgagatggaaagctgcctgagcggcaacatctctcagtgccctagaaccaccgtgacctccgacatcgtgcccagatacgcctttgtgaatggcggcgtggtggccaactgcatcaccaccacctgtacctgcaacggcatcggcaaccggatcaaccagcctccagatcagggcgtgaagattatcacccacaaagagtgtaacaccatcggcatcaacggcatgctgttcaataccaacaaagagggcaccctggccttctacacccctgacgatatcaccctgaacaacagcgtggccctggatcctatcgacatctccatcgagctgaacaaggccaagagcgacctggaagaagccaaagagtggatccggcgggccaaccagaagctggatagcatcggaagctggcaccagagcagcaccaccatcatcgtgatcctgattatgatgattatcctgttcatcatcaacattaccatcatcacgatcgccatcaagtactaccggatccagaaacggaaccgcgtggaccagaatgacaagccctacgtgctgacaaacaag

SEQ ID NO: 413 - RNA, 1617 nt, synthetic construct
  source 1..1617: mol_type = other RNA
atgctgatctccatcctgctgatcatcaccacaatgatcatggccagccactgccagatcgacatcaccaagctgcagcacgtgggcgtgctggtcaatagccctaagggcatgaagatcagccagaacttcgagacacgctacctgatcctgtctctgatccccaagatcgaggacagcaacagctgcggcgaccagcagatcaagcagtacaagcggctgctggacagactgatcatccctctgtacgacggcctgcggctgcagaaagatgtgatcgtgaccaatcaagagagcaacgagaacacagaccccagaaccgagagattcttcggcggcgtgatcggcacaatcgccctgggagttgctacaagcgcccagattacagccgccgtggctctggtggaagccaagcaggccagaagcgacatcgagaagctgaaagaggccatccgggacaccaacaaggccgtgcagtctgtgcaaagcagcgtgggcaatctgatcgtggccattaagagcgtgcaggactacgtgaacaaagagatcgtcccctctatcgccagactgggatgtgaagctgccggactgcagctgggaattgccctgacacagcactacagcgagctgaccaacatcttcggcgacaacatcggcagcctgcaagagaagggcattaagctgcaggccatcgccagcctgtaccgcaccaacatcaccgagatcttcaccaccagcaccgtggataagtacgacatctacgacctgctgttcaccgagagcatcaaagtgcgcgtgatcgacgtggacctgaacgactacagcatcaccctgcaagtgcggctgcctctgctgaccagactgctgaacacccagatctacaaggtggacagcatctcctacaacatccagaaccgcgagtggtacatcccactgcctagccacattatgaccaagggcgcctttctcggcggagccgatgtgaaagagtgcatcgaggccttcagcagctacatctgccctagcgatcctggcttcgtgctgaaccacgagatggaaagctgcctgagcggcaacatctctcagtgccctagaaccaccgtgacctccgacatcgtgcccagatacgcctttgtgaatggcggcgtggtggccaactgcatcaccaccacctgtacctgcaacggcatcggcaaccggatcaaccagcctccagatcagggcgtgaagattatcacccacaaagagtgtaacaccatcggcatcaacggcatgctgttcaataccaacaaagagggcaccctggccttctacacccctgacgatatcaccctgaacaacagcgtggccctggatcctatcgacatctccatcgagctgaacaaggccaagagcgacctggaagaagccaaagagtggatccggcgggccaaccagaagctggatagcatcggaagctggcaccagagcagcaccaccatcatcgtgatcctgattatgatgattatcctgttcatcatcaacattaccatcatcacgatcgccatcaagtactaccggatccagaaacggaaccgcgtggaccagaatgacaagccctacgtgctgacaaacaag

SEQ ID NO: 414 - DNA, 1617 bp, synthetic construct
  source 1..1617: mol_type = other DNA
atgctgatctccatcctgctgatcatcaccacaatgatcatggccagccactgccagatcgacatcaccaagctgcagcacgtgggcgtgctggtcaatagccctaagggcatgaagatcagccagaacttcgagacacgctacctgatcctgtctctgatccccaagatcgaggacagcaacagctgcggcgaccagcagatcaagcagtacaagcggctgctggacagactgatcatccctctgtacgacggcctgcggctgcagaaagatgtgatcgtgaccaatcaagagagcaacgagaacacagaccccagaaccgagagattcttcggcggcgtgatcggcacaatcgccctgggagttgctacaagcgcccagattacagccgccgtggctctggtggaagccaagcaggccagaagcgacatcgagaagctgaaagaggccatccgggacaccaacaaggccgtgcagtgcgtgcaaagcagcgtgggcaatctgatctgcgccattaagagcgtgcaggactacgtgaacaaagagatcgtcccctctatcgccagactgggatgtgaagctgccggactgcagctgggaattgccctgacacagcactacagcgagctgaccaacatcttcggcgacaacatcggcagcctgcaagagaagggcattaagctgcaggccatcgccagcctgtaccgcaccaacatcaccgagatcttcaccaccagcaccgtggataagtacgacatctacgacctgctgttcaccgagagcatcaaagtgcgcgtgatcgacgtggacctgaacgactacagcatcaccctgcaagtgcggctgcctctgctgaccagactgctgaacacccagatctacaaggtggacagcatctcctacaacatccagaaccgcgagtggtacatcccactgcctagccacattatgaccaagggcgcctttctcggcggagccgatgtgaaagagtgcatcgaggccttcagcagctacatctgccctagcgatcctggcttcgtgctgaaccacgagatggaaagctgcctgagcggcaacatctctcagtgccctagaaccaccgtgacctccgacatcgtgcccagatacgcctttgtgaatggcggcgtggtggccaactgcatcaccaccacctgtacctgcaacggcatcggcaaccggatcaaccagcctccagatcagggcgtgaagattatcacccacaaagagtgtaacaccatcggcatcaacggcatgctgttcaataccaacaaagagggcaccctggccttctacacccctgacgatatcaccctgaacaacagcgtggccctggatcctatcgacatctccatcgagctgaacaaggccaagagcgacctggaagaagccaaagagtggatccggcgggccaaccagaagctggatagcatcggaagctggcaccagagcagcaccaccatcatcgtgatcctgattatgatgattatcctgttcatcatcaacattaccatcatcacgatcgccatcaagtactaccggatccagaaacggaaccgcgtggaccagaatgacaagccctacgtgctgacaaacaag

SEQ ID NO: 415 - RNA, 1617 nt, synthetic construct
  source 1..1617: mol_type = other RNA
atgctgatctccatcctgctgatcatcaccacaatgatcatggccagccactgccagatcgacatcaccaagctgcagcacgtgggcgtgctggtcaatagccctaagggcatgaagatcagccagaacttcgagacacgctacctgatcctgtctctgatccccaagatcgaggacagcaacagctgcggcgaccagcagatcaagcagtacaagcggctgctggacagactgatcatccctctgtacgacggcctgcggctgcagaaagatgtgatcgtgaccaatcaagagagcaacgagaacacagaccccagaaccgagagattcttcggcggcgtgatcggcacaatcgccctgggagttgctacaagcgcccagattacagccgccgtggctctggtggaagccaagcaggccagaagcgacatcgagaagctgaaagaggccatccgggacaccaacaaggccgtgcagtgcgtgcaaagcagcgtgggcaatctgatctgcgccattaagagcgtgcaggactacgtgaacaaagagatcgtcccctctatcgccagactgggatgtgaagctgccggactgcagctgggaattgccctgacacagcactacagcgagctgaccaacatcttcggcgacaacatcggcagcctgcaagagaagggcattaagctgcaggccatcgccagcctgtaccgcaccaacatcaccgagatcttcaccaccagcaccgtggataagtacgacatctacgacctgctgttcaccgagagcatcaaagtgcgcgtgatcgacgtggacctgaacgactacagcatcaccctgcaagtgcggctgcctctgctgaccagactgctgaacacccagatctacaaggtggacagcatctcctacaacatccagaaccgcgagtggtacatcccactgcctagccacattatgaccaagggcgcctttctcggcggagccgatgtgaaagagtgcatcgaggccttcagcagctacatctgccctagcgatcctggcttcgtgctgaaccacgagatggaaagctgcctgagcggcaacatctctcagtgccctagaaccaccgtgacctccgacatcgtgcccagatacgcctttgtgaatggcggcgtggtggccaactgcatcaccaccacctgtacctgcaacggcatcggcaaccggatcaaccagcctccagatcagggcgtgaagattatcacccacaaagagtgtaacaccatcggcatcaacggcatgctgttcaataccaacaaagagggcaccctggccttctacacccctgacgatatcaccctgaacaacagcgtggccctggatcctatcgacatctccatcgagctgaacaaggccaagagcgacctggaagaagccaaagagtggatccggcgggccaaccagaagctggatagcatcggaagctggcaccagagcagcaccaccatcatcgtgatcctgattatgatgattatcctgttcatcatcaacattaccatcatcacgatcgccatcaagtactaccggatccagaaacggaaccgcgtggaccagaatgacaagccctacgtgctgacaaacaag

SEQ ID NO: 416 - DNA, 1617 bp, synthetic construct
  source 1..1617: mol_type = other DNA
atgctgatctccatcctgctgatcatcaccacaatgatcatggccagccactgccagatcgacatcaccaagctgcagcacgtgggcgtgctggtcaatagccctaagggcatgaagatcagccagaacttcgagacacgctacctgatcctgtctctgatccccaagatcgaggacagcaacagctgcggcgaccagcagatcaagcagtacaagcggctgctggacagactgatcatccctctgtacgacggcctgcggctgcagaaagatgtgatcgtgaccaatcaagagagcaacgagaacacagaccccagaaccgagagattcttcggcggcgtgatcggcacaatcgccctgggagttgctacaagcgcccagattacagccgccgtggctctggtggaagccaagcaggccagaagcgacatcgagaagctgaaagaggccatccgggacaccaacaaggccgtgcagtgcgtgcaaagcagcgtgggcaatctgatctgcgccattaagagcgtgcaggactacgtgaacaaagagatcgtcccctctatcgccagactgggatgtgaagctgccggactgcagctgggaattgccctgacacagcactacagcgagctgaccaacatcttcggcgacaacatcggcagcctgcaagagaagggcattaagctgcaggccatcgccagcctgtaccgcaccaacatcaccgagatcttcaccaccagcaccgtggataagtacgacatctacgacctgctgttcaccgagagcatcaaagtgcgcgtgatcgacgtggacctgaacgactacagcatcaccctgcaagtgcggctgcctctgctgaccagactgctgaacacccagatctacaaggtggacagcatctcctacaacatccagaaccgcgagtggtacatcccactgcctagccacattatgaccaagggcgcctttctcggcggagccgatgtgaaagagtgcatcgaggccttcagcagctacatctgccctagcgatcctggcttcgtgctgaaccacgagatggaaagctgcctgagcggcaacatctctcagtgccctagaaccaccgtgacctccgacatcgtgcccagatacgcctttgtgaatggcggcgtggtggccaactgcatcaccaccacctgtacctgcaacggcatcggcaaccggatcaaccagcctccagatcagggcgtgaagattatcacccacaaagagtgtaacaccatcggcatcaacggcatgctgttcaataccaacaaagagggcaccctggccttctacacccctgacgatatcaccctgaacaacagcgtggccctggatcctatcgacatctccatcgagctgaacaagctcaagagcgacctggaagaaagcaaagagtggatccggcggagcaaccagaagctggatagcatcggaagctggcaccagagcagcaccaccatcatcgtgatcctgattatgatgattatcctgttcatcatcaacattaccatcatcacgatcgccatcaagtactaccggatccagaaacggaaccgcgtggaccagaatgacaagccctacgtgctgacaaacaag

SEQ ID NO: 417 - RNA, 1617 nt, synthetic construct
  source 1..1617: mol_type = other RNA
atgctgatctccatcctgctgatcatcaccacaatgatcatggccagccactgccagatcgacatcaccaagctgcagcacgtgggcgtgctggtcaatagccctaagggcatgaagatcagccagaacttcgagacacgctacctgatcctgtctctgatccccaagatcgaggacagcaacagctgcggcgaccagcagatcaagcagtacaagcggctgctggacagactgatcatccctctgtacgacggcctgcggctgcagaaagatgtgatcgtgaccaatcaagagagcaacgagaacacagaccccagaaccgagagattcttcggcggcgtgatcggcacaatcgccctgggagttgctacaagcgcccagattacagccgccgtggctctggtggaagccaagcaggccagaagcgacatcgagaagctgaaagaggccatccgggacaccaacaaggccgtgcagtgcgtgcaaagcagcgtgggcaatctgatctgcgccattaagagcgtgcaggactacgtgaacaaagagatcgtcccctctatcgccagactgggatgtgaagctgccggactgcagctgggaattgccctgacacagcactacagcgagctgaccaacatcttcggcgacaacatcggcagcctgcaagagaagggcattaagctgcaggccatcgccagcctgtaccgcaccaacatcaccgagatcttcaccaccagcaccgtggataagtacgacatctacgacctgctgttcaccgagagcatcaaagtgcgcgtgatcgacgtggacctgaacgactacagcatcaccctgcaagtgcggctgcctctgctgaccagactgctgaacacccagatctacaaggtggacagcatctcctacaacatccagaaccgcgagtggtacatcccactgcctagccacattatgaccaagggcgcctttctcggcggagccgatgtgaaagagtgcatcgaggccttcagcagctacatctgccctagcgatcctggcttcgtgctgaaccacgagatggaaagctgcctgagcggcaacatctctcagtgccctagaaccaccgtgacctccgacatcgtgcccagatacgcctttgtgaatggcggcgtggtggccaactgcatcaccaccacctgtacctgcaacggcatcggcaaccggatcaaccagcctccagatcagggcgtgaagattatcacccacaaagagtgtaacaccatcggcatcaacggcatgctgttcaataccaacaaagagggcaccctggccttctacacccctgacgatatcaccctgaacaacagcgtggccctggatcctatcgacatctccatcgagctgaacaagctcaagagcgacctggaagaaagcaaagagtggatccggcggagcaaccagaagctggatagcatcggaagctggcaccagagcagcaccaccatcatcgtgatcctgattatgatgattatcctgttcatcatcaacattaccatcatcacgatcgccatcaagtactaccggatccagaaacggaaccgcgtggaccagaatgacaagccctacgtgctgacaaacaag

SEQ ID NO: 418 - DNA, 1617 bp, synthetic construct
  source 1..1617: mol_type = other DNA
atgctgatctccatcctgctgatcatcaccacaatgatcatggccagccactgccagatcgacatcaccaagctgcagcacgtgggcgtgctggtcaatagccctaagggcatgaagatcagccagaacttcgagacacgctacctgatcctgtctctgatccccaagatcgaggacagcaacagctgcggcgaccagcagatcaagcagtacaagcggctgctggacagactgatcatccctctgtacgacggcctgcggctgcagaaagatgtgatcgtgaccaatcaagagagcaacgagaacacagaccccagaaccgagagattcttcggcggcgtgatcggcacaatcgccctgggagttgctacaagcgcccagattacagccgccgtggctctggtggaagccaagcaggccagaagcgacatcgagaagctgaaagaggccatccgggacaccaacaaggccgtgcagtgcgtgcaaagcagcgtgggcaatctgatctgcgccattaagagcgtgcaggactacgtgaacaaagagatcgtcccctctatcgccagactgggatgtgaagctgccggactgcagctgggaattgccctgacacagcactacagcgagctgaccaacatcttcggcgacaacatcggcagcctgcaagagaagggcattaagctgcaggccatcgccagcctgtaccgcaccaacatcaccgagatcttcaccaccagcaccgtggataagtacgacatctacgacctgctgttcaccgagagcatcaaagtgcgcgtgatcgacgtggacctgaacgactacagcatcaccctgcaagtgcggctgcctctgctgaccagactgctgaacacccagatctacaaggtggacagcatctcctacaacatccagaaccgcgagtggtacatcccactgcctagccacattatgaccaagggcgcctttctcggcggagccgatgtgaaagagtgcatcgaggccttcagcagctacatctgccctagcgatcctggcttcgtgctgaaccacgagatggaaagctgcctgagcggcaacatctctcagtgccctagaaccaccgtgacctccgacatcgtgcccagatacgcctttgtgaatggcggcgtggtggccaactgcatcaccaccacctgtacctgcaacggcatcggcaaccggatcaaccagcctccagatcagggcgtgaagattatcacccacaaagagtgtaacaccatcggcatcaacggcatgctgttcaataccaacaaagagggcaccctggccttctacacccctgacgatatcaccctgaacaacagcgtggccctggatcctatcgacatctccatcgagctgaacaagctcaagagcgacctggaagaagccaaagagtggatccggcgggccaaccagaagctggatagcatcggaagctggcaccagagcagcaccaccatcatcgtgatcctgattatgatgattatcctgttcatcatcaacattaccatcatcacgatcgccatcaagtactaccggatccagaaacggaaccgcgtggaccagaatgacaagccctacgtgctgacaaacaag

SEQ ID NO: 419 - RNA, 1617 nt, synthetic construct
  source 1..1617: mol_type = other RNA
atgctgatctccatcctgctgatcatcaccacaatgatcatggccagccactgccagatcgacatcaccaagctgcagcacgtgggcgtgctggtcaatagccctaagggcatgaagatcagccagaacttcgagacacgctacctgatcctgtctctgatccccaagatcgaggacagcaacagctgcggcgaccagcagatcaagcagtacaagcggctgctggacagactgatcatccctctgtacgacggcctgcggctgcagaaagatgtgatcgtgaccaatcaagagagcaacgagaacacagaccccagaaccgagagattcttcggcggcgtgatcggcacaatcgccctgggagttgctacaagcgcccagattacagccgccgtggctctggtggaagccaagcaggccagaagcgacatcgagaagctgaaagaggccatccgggacaccaacaaggccgtgcagtgcgtgcaaagcagcgtgggcaatctgatctgcgccattaagagcgtgcaggactacgtgaacaaagagatcgtcccctctatcgccagactgggatgtgaagctgccggactgcagctgggaattgccctgacacagcactacagcgagctgaccaacatcttcggcgacaacatcggcagcctgcaagagaagggcattaagctgcaggccatcgccagcctgtaccgcaccaacatcaccgagatcttcaccaccagcaccgtggataagtacgacatctacgacctgctgttcaccgagagcatcaaagtgcgcgtgatcgacgtggacctgaacgactacagcatcaccctgcaagtgcggctgcctctgctgaccagactgctgaacacccagatctacaaggtggacagcatctcctacaacatccagaaccgcgagtggtacatcccactgcctagccacattatgaccaagggcgcctttctcggcggagccgatgtgaaagagtgcatcgaggccttcagcagctacatctgccctagcgatcctggcttcgtgctgaaccacgagatggaaagctgcctgagcggcaacatctctcagtgccctagaaccaccgtgacctccgacatcgtgcccagatacgcctttgtgaatggcggcgtggtggccaactgcatcaccaccacctgtacctgcaacggcatcggcaaccggatcaaccagcctccagatcagggcgtgaagattatcacccacaaagagtgtaacaccatcggcatcaacggcatgctgttcaataccaacaaagagggcaccctggccttctacacccctgacgatatcaccctgaacaacagcgtggccctggatcctatcgacatctccatcgagctgaacaagctcaagagcgacctggaagaagccaaagagtggatccggcgggccaaccagaagctggatagcatcggaagctggcaccagagcagcaccaccatcatcgtgatcctgattatgatgattatcctgttcatcatcaacattaccatcatcacgatcgccatcaagtactaccggatccagaaacggaaccgcgtggaccagaatgacaagccctacgtgctgacaaacaag

SEQ ID NO: 420 - protein, 539 aa, synthetic construct
MLISILLIITTMIMASHCQIDITKLQHVGVLVNSPKGMKISQNFETRYLILSLIPKIEDSNSCGDQQIKQYKRLLDRLIIPLYDGLRLQKDVIVTNQESNENTDPRTERFFGGVIGTIALGVATSAQITAAVALVEAKQARSDIEKLKEAIRDTNKAVQSVQSSVGNLIVAIKSVQDYVNKEIVPSIARLGCEAAGLQLGIALTQHYSELTNIFGDNIGSLQEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALDPIDISIELNKAKSDLEESKEWIRRSNQKLDSIGGGSGYIPEAPRDGQAYVRKDGEWVLLSTFLGRSLEVLFQGPGSAWSHPQFEKAG

SEQ ID NO: 421-422 - skipped (2 entries)

SEQ ID NO: 423 - protein, 372 aa, synthetic construct
FFGGVIGTIALGVATSAQITAAVALVEAKQARSDIEKLKEAIRDTNKAVQSVQSSVGNLIVAIKSVQDYVNKEIVPSIARLGCEAAGLQLGIALTQHYSCLTNIFGDNIGSLQEKGIKLQGIASCYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALDPIDISIELNKAKSDLEESKEWIRRSNQKL

SEQ ID NO: 424 - protein, 91 aa, synthetic construct; identical to SEQ ID NO: 307

SEQ ID NO: 425 - protein, 372 aa, synthetic construct
FFGGVIGTIALGVATSAQITAAVALVEAKQARSDIEKLKEAIRDTNKAVQSVQSSVGNLIVAIKSVQDYVNKEIVPSIARLGCEAAGLQLGIALTQHYSCLTNIFGDNIGSLQEKGIKLQGIACLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALDPIDISIELNKAKSDLEESKEWIRRSNQKL

SEQ ID NO: 426 - protein, 91 aa, synthetic construct; identical to SEQ ID NO: 307

SEQ ID NO: 427 - protein, 372 aa, synthetic construct
FFGGVIGTIALGVATSAQITAAVALVEAKQARSDIEKLKEAIRDTNKAVQSVQSSVGNLIVAIKSVQDYVNKEIVPSIARLGCEAAGLQLGIALTQHYSCLTNIFGDNIGSLQEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALDPIDISIELNKAKSDLEESKEWIRRSNQKL

SEQ ID NO: 428 - protein, 91 aa, synthetic construct
QIDITKLQHVGVLVNSPKGMKISQNFETRYLILSLIPKIEDSNSCGDQQIKQYKRLLDRLIIPLYDCLRLQKDVIVTNQESNENTDPRTER

SEQ ID NO: 429 - protein, 372 aa, synthetic construct
FFGGVIGTIALGVATSAQITAAVALVEAKQARSDIEKLKEAIRDTNKAVQSVQSSVGNLIVAIKSVQDYVNKEIVPSIARLGCEAAGLQLGIALTQHYSELTNIFGDNIGSLQEKGIKLQGIASLYRTNITEIFTTSTVDKYDIYDLLFTESIKVRVIDVDLNDYSIVLQVRLPLLTRLLNTQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALDPIDISIELNKAKSDLEESKEWIRRSNQKL

SEQ ID NO: 430 - protein, 91 aa, synthetic construct; identical to SEQ ID NO: 307

SEQ ID NO: 431 - protein, 372 aa, synthetic construct
FFGGVIGTIALGVATSAQITAAVALVEAKQARSDIEKLKEAIRDTNKAVQSVQSSVGNLIVAIKSVQDYVNKEIVPSIARLGCEAAGLQLGIALTQHYSELTNIFGDNIGSLQEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALDPIDISIELNKLKSDLEELKEWIRRSNQKL

SEQ ID NO: 432 - protein, 91 aa, synthetic construct; identical to SEQ ID NO: 307

SEQ ID NO: 433 - protein, 372 aa, synthetic construct
FFGGVIGTIALGVATSAQITAAVALVEAKQARSDIEKLKEAIRDTNKAVQSVQSSVGNLIVAIKSVQDYVNKEIVPSIARLGCEAAGLQLGIALTQHYSELTNIFGDNIGSLQEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALDPIDISIELNKLKSDLEESKEWFRRSNQKL

SEQ ID NO: 434 - protein, 91 aa, synthetic construct; identical to SEQ ID NO: 307

SEQ ID NO: 435 - protein, 372 aa, synthetic construct
FFGGVIGTIALGVATSAQITAAVALVEAKQARSDIEKLKEAIRDTNKAVQSVQSSVGNLIVAIKSVQDYVNKEIVPSIARLGCEAAGLQLGIALTQHYSELTNIFGDNIGSLQEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALDPIDISIELNKIKSDLEEIKEWIRRSNQKL

SEQ ID NO: 436 - protein, 91 aa, synthetic construct; identical to SEQ ID NO: 307

SEQ ID NO: 437 - protein, 372 aa, synthetic construct
FFGGVIGTIALGVATSAQITAAVALVEAKQARSDIEKLKEAIRDTNKAVQCVQSSVGNLICAIKSVQDYVNKEIVPSIARLGCEAAGLQLGIALTQHYSCLTNIFGDNIGSLQEKGIKLQGIASCYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALDPIDISIELNKLKSDLEELKEWIRRSNQKL

SEQ ID NO: 438 - protein, 91 aa, synthetic construct; identical to SEQ ID NO: 307

SEQ ID NO: 439 - protein, 372 aa, synthetic construct
FFGGVIGTIALGVATSAQITAAVALVEAKQARSDIEKLKEAIRDTNKAVQCVQSSVGNLICAIKSVQDYVNKEIVPSIARLGCEAAGLQLGIALTQHYSCLTNIFGDNIGSLQEKGIKLQGIASCYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALDPIDISIELNKLKSDLEESKEWFRRSNQKL

SEQ ID NO: 440 - protein, 91 aa, synthetic construct; identical to SEQ ID NO: 307

SEQ ID NO: 441 - protein, 372 aa, synthetic construct
FFGGVIGTIALGVATSAQITAAVALVEAKQARSDIEKLKEAIRDTNKAVQCVQSSVGNLICAIKSVQDYVNKEIVPSIARLGCEAAGLQLGIALTQHYSCLTNIFGDNIGSLQEKGIKLQGIACLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALDPIDISIELNKLKSDLEELKEWIRRSNQKL

SEQ ID NO: 442 - protein, 91 aa, synthetic construct; identical to SEQ ID NO: 307

SEQ ID NO: 443 - protein, 372 aa, synthetic construct
FFGGVIGTIALGVATSAQITAAVALVEAKQARSDIEKLKEAIRDTNKAVQCVQSSVGNLICAIKSVQDYVNKEIVPSIARLGCEAAGLQLGIALTQHYSCLTNIFGDNIGSLQEKGIKLQGIACLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALDPIDISIELNKLKSDLEESKEWFRRSNQKL

SEQ ID NO: 444 - protein, 91 aa, synthetic construct; identical to SEQ ID NO: 307

SEQ ID NO: 445 - protein, 372 aa, synthetic construct
FFGGVIGTIALGVATSAQITAAVALVEAKQARSDIEKLKEAIRDTNKAVQCVQSSVGNLICAIKSVQDYVNKEIVPSIARLGCEAAGLQLGIALTQHYSCLTNIFGDNIGSLQEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALDPIDISIELNKLKSDLEELKEWIRRSNQKL

SEQ ID NO: 446 - protein, 91 aa, synthetic construct; identical to SEQ ID NO: 428

SEQ ID NO: 447 - protein, 372 aa, synthetic construct
FFGGVIGTIALGVATSAQITAAVALVEAKQARSDIEKLKEAIRDTNKAVQCVQSSVGNLICAIKSVQDYVNKEIVPSIARLGCEAAGLQLGIALTQHYSCLTNIFGDNIGSLQEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALDPIDISIELNKLKSDLEESKEWFRRSNQKL

SEQ ID NO: 448 - protein, 91 aa, synthetic construct; identical to SEQ ID NO: 428

SEQ ID NO: 449 - protein, 372 aa, synthetic construct
FFGGVIGTIALGVATSAQITAAVALVEAKQARSDIEKLKEAIRDTNKAVQCVQSSVGNLICAIKSVQDYVNKEIVPSIARLGCEAAGLQLGIALTQHYSCLTNIFGDNIGSLQEKGIKLQGIASCYRTNITEIFTTSTVDKYDIYDLLFTESIKVRVIDVDLNDYSIVLQVRLPLLTRLLNTQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALDPIDISIELNKLKSDLEELKEWIRRSNQKL

SEQ ID NO: 450 - protein, 91 aa, synthetic construct; identical to SEQ ID NO: 307

SEQ ID NO: 451 - protein, 372 aa, synthetic construct
FFGGVIGTIALGVATSAQITAAVALVEAKQARSDIEKLKEAIRDTNKAVQCVQSSVGNLICAIKSVQDYVNKEIVPSIARLGCEAAGLQLGIALTQHYSCLTNIFGDNIGSLQEKGIKLQGIASCYRTNITEIFTTSTVDKYDIYDLLFTESIKVRVIDVDLNDYSIVLQVRLPLLTRLLNTQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALDPIDISIELNKLKSDLEESKEWFRRSNQKL

SEQ ID NO: 452 - protein, 91 aa, synthetic construct; identical to SEQ ID NO: 307

SEQ ID NO: 453 - protein, 372 aa, synthetic construct
FFGGVIGTIALGVATSAQITAAVALVEAKQARSDIEKLKEAIRDTNKAVQCVQSSVGNLICAIKSVQDYVNKEIVPSIARLGCEAAGLQLGIALTQHYSCLTNIFGDNIGSLQEKGIKLQGIACLYRTNITEIFTTSTVDKYDIYDLLFTESIKVRVIDVDLNDYSIVLQVRLPLLTRLLNTQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALDPIDISIELNKLKSDLEELKEWIRRSNQKL

SEQ ID NO: 454 - protein, 91 aa, synthetic construct; identical to SEQ ID NO: 307

SEQ ID NO: 455 - protein, 372 aa, synthetic construct
FFGGVIGTIALGVATSAQITAAVALVEAKQARSDIEKLKEAIRDTNKAVQCVQSSVGNLICAIKSVQDYVNKEIVPSIARLGCEAAGLQLGIALTQHYSCLTNIFGDNIGSLQEKGIKLQGIACLYRTNITEIFTTSTVDKYDIYDLLFTESIKVRVIDVDLNDYSIVLQVRLPLLTRLLNTQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALDPIDISIELNKLKSDLEESKEWFRRSNQKL

SEQ ID NO: 456 - protein, 91 aa, synthetic construct; identical to SEQ ID NO: 307

SEQ ID NO: 457 - protein, 372 aa, synthetic construct
FFGGVIGTIALGVATSAQITAAVALVEAKQARSDIEKLKEAIRDTNKAVQCVQSSVGNLICAIKSVQDYVNKEIVPSIARLGCEAAGLQLGIALTQHYSCLTNIFGDNIGSLQEKGIKLQGIASLYRTNITEIFTTSTVDKYDIYDLLFTESIKVRVIDVDLNDYSIVLQVRLPLLTRLLNTQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALDPIDISIELNKLKSDLEESKEWFRRSNQKL

SEQ ID NO: 458 - protein, 91 aa, synthetic construct; identical to SEQ ID NO: 428

SEQ ID NO: 459 - protein, 372 aa, synthetic construct
FFGGVIGTIALGVATSAQITAAVALVEAKQARSDIEKLKEAIRDTNKAVQCVQSSVGNLICAIKSVQDYVNKEIVPSIARLGCEAAGLQLGIALTQHYSCLTNIFGDNIGSLQEKGIKLQGIASCYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALDPISISIELNKLKSDLEELKEWIRRSNQKL

SEQ ID NO: 460 - protein, 91 aa, synthetic construct; identical to SEQ ID NO: 307

SEQ ID NO: 461 - protein, 372 aa, synthetic construct
FFGGVIGTIALGVATSAQITAAVALVEAKQARSDIEKLKEAIRDTNKAVQCVQSSVGNLICAIKSVQDYVNKEIVPSIARLGCEAAGLQLGIALTQHYSCLTNIFGDNIGSLQEKGIKLQGIACLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALDPISISIELNKLKSDLEELKEWIRRSNQKL

SEQ ID NO: 462 - protein, 91 aa, synthetic construct; identical to SEQ ID NO: 307

SEQ ID NO: 463 - protein, 372 aa, synthetic construct
FFGGVIGTIALGVATSAQITAAVALVEAKQARSDIEKLKEAIRDTNKAVQCVQSSVGNLICAIKSVQDYVNKEIVPSIARLGCEAAGLQLGIALTQHYSCLTNIFGDNIGSLQEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALDPISISIELNKLKSDLEELKEWIRRSNQKL

SEQ ID NO: 464 - protein, 91 aa, synthetic construct; identical to SEQ ID NO: 428

SEQ ID NO: 465 - protein, 372 aa, synthetic construct
FFGGVIGTIALGVATSAQITAAVALVEAKQARSDIEKLKEAIRDTNKAVQCVQSSVGNLICAIKSVQDYVNKEIVPSIARLGCEAAGLQLGIALTQHYSCLTNIFGDNIGSLQEKGIKLQGIASCYRTNITEIFTTSTVDKYDIYDLLFTESIKVRVIDVDLNDYSIVLQVRLPLLTRLLNTQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALDPISISIELNKLKSDLEELKEWIRRSNQKL

SEQ ID NO: 466 - protein, 91 aa, synthetic construct; identical to SEQ ID NO: 307

SEQ ID NO: 467 - protein, 372 aa, synthetic construct
FFGGVIGTIALGVATSAQITAAVALVEAKQARSDIEKLKEAIRDTNKAVQCVQSSVGNLICAIKSVQDYVNKEIVPSIARLGCEAAGLQLGIALTQHYSCLTNIFGDNIGSLQEKGIKLQGIACLYRTNITEIFTTSTVDKYDIYDLLFTESIKVRVIDVDLNDYSIVLQVRLPLLTRLLNTQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALDPISISIELNKLKSDLEELKEWIRRSNQKL

SEQ ID NO: 468 - protein, 91 aa, synthetic construct; identical to SEQ ID NO: 307

SEQ ID NO: 469 - protein, 372 aa, synthetic construct
FFGGVIGTIALGVATSAQITAAVALVEAKQARSDIEKLKEAIRDTNKAVQCVQSSVGNLICAIKSVQDYVNKEIVPSIARLGCEAAGLQLGIALTQHYSCLTNIFGDNIGSLQEKGIKLQGIASLYRTNITEIFTTSTVDKYDIYDLLFTESIKVRVIDVDLNDYSIVLQVRLPLLTRLLNTQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALDPISISIELNKLKSDLEELKEWIRRSNQKL

SEQ ID NO: 470 - protein, 91 aa, synthetic construct; identical to SEQ ID NO: 428

SEQ ID NO: 471 - protein, 372 aa, synthetic construct
FFGGVIGTIALGVATSAQITAAVALVEAKQARSDIEKLKEAIRDTNKAVQCVQSSVGNLICAIKSVQDYVNKEIVPSIARLGCEAAGLQLGIALTQHYSELTNIFGDNIGSLQEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALDPIDISIELNKLKSDLEELKEWIRRSNQKL

SEQ ID NO: 472 - protein, 91 aa, synthetic construct; identical to SEQ ID NO: 307

SEQ ID NO: 473 - protein, 372 aa, synthetic construct
FFGGVIGTIALGVATSAQITAAVALVEAKQARSDIEKLKEAIRDTNKAVQCVQSSVGNLICAIKSVQDYVNKEIVPSIARLGCEAAGLQLGIALTQHYSELTNIFGDNIGSLQEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALDPIDISIELNKAKSDLEELKEWIRRSNQKL

SEQ ID NO: 474 - protein, 91 aa, synthetic construct; identical to SEQ ID NO: 307

SEQ ID NO: 475 - protein, 372 aa, synthetic construct; identical to SEQ ID NO: 437

SEQ ID NO: 476 - protein, 91 aa, synthetic construct; identical to SEQ ID NO: 335

SEQ ID NO: 477 - protein, 372 aa, synthetic construct; identical to SEQ ID NO: 441

SEQ ID NO: 478 - protein, 91 aa, synthetic construct; identical to SEQ ID NO: 335

SEQ ID NO: 479 - protein, 372 aa, synthetic construct; identical to SEQ ID NO: 445

SEQ ID NO: 480 - protein, 91 aa, synthetic construct
QIDITKLQHVGVLVNSPKGMKISQNFETRYLILSLIPKIEDSNSCGDQQIKQYKRLLDRLIIPLYDCLRLQKDVIVTNQESNENTDPGSAS

SEQ ID NO: 481 - protein, 372 aa, synthetic construct
GSGGVIGTIALGVATSAQITAAVALVEAKQARSDIEKLKEAIRDTNKAVQCVQSSVGNLICAIKSVQDYVNKEIVPSIARLGCEAAGLQLGIALTQHYSCLTNIFGDNIGSLQEKGIKLQGIASCYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALDPIDISIELNKLKSDLEELKEWIRRSNQKL

SEQ ID NO: 482 - protein, 91 aa, synthetic construct; identical to SEQ ID NO: 307

SEQ ID NO: 483 - protein, 372 aa, synthetic construct
GSGGVIGTIALGVATSAQITAAVALVEAKQARSDIEKLKEAIRDTNKAVQCVQSSVGNLICAIKSVQDYVNKEIVPSIARLGCEAAGLQLGIALTQHYSCLTNIFGDNIGSLQEKGIKLQGIACLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALDPIDISIELNKLKSDLEELKEWIRRSNQKL

SEQ ID NO: 484 - protein, 91 aa, synthetic construct; identical to SEQ ID NO: 307

SEQ ID NO: 485 - protein, 372 aa, synthetic construct
GSGGVIGTIALGVATSAQITAAVALVEAKQARSDIEKLKEAIRDTNKAVQCVQSSVGNLICAIKSVQDYVNKEIVPSIARLGCEAAGLQLGIALTQHYSELTNIFGDNIGSLQEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALDPIDISIELNKLKSDLEELKEWIRRSNQKL

SEQ ID NO: 486 - protein, 91 aa, synthetic construct; identical to SEQ ID NO: 307

SEQ ID NO: 487 - protein, 372 aa, synthetic construct
GSGGVIGTIALGVATSAQITAAVALVEAKQARSDIEKLKEAIRDTNKAVQCVQSSVGNLICAIKSVQDYVNKEIVPSIARLGCEAAGLQLGIALTQHYSELTNIFGDNIGSLQEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALDPIDISIELNKAKSDLEELKEWIRRSNQKL

SEQ ID NO: 488 - protein, 91 aa, synthetic construct; identical to SEQ ID NO: 307

SEQ ID NO: 489 - protein, 375 aa, synthetic construct
FFGGVIGTIALGVATSAQITAAVALVEAKQARSDIEKLKEAIRDTNKAVQCVQSSVGNLICAIKSVQDYVNKEIVPSIARLGCEAAGLQLGIALTQHYSELTNIFGDNIGSLQEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALDPIDISIELNKLKSDLEELKEWIRRSNQKLDSI

SEQ ID NO: 490 - protein, 91 aa, synthetic construct; identical to SEQ ID NO: 307

SEQ ID NO: 491 - protein, 375 aa, synthetic construct
FFGGVIGTIALGVATSAQITAAVALVEAKQARSDIEKLKEAIRDTNKAVQCVQSSVGNLICAIKSVQDYVNKEIVPSIARLGCEAAGLQLGIALTQHYSELTNIFGDNIGSLQEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALDPIDISIELNKLKSDLEESKEWIRRLNQKLDSI

SEQ ID NO: 492 - protein, 91 aa, synthetic construct; identical to SEQ ID NO: 307

SEQ ID NO: 493 - protein, 375 aa, synthetic construct
FFGGVIGTIALGVATSAQITAAVALVEAKQARSDIEKLKEAIRDTNKAVQCVQSSVGNLICAIKSVQDYVNKEIVPSIARLGCEAAGLQLGIALTQHYSCLTNIFGDNIGSLQEKGIKLQGIASCYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALDPIDISIELNKLKSDLEELKEWIRRSNQKLDSI

SEQ ID NO: 494 - protein, 91 aa, synthetic construct; identical to SEQ ID NO: 307

SEQ ID NO: 495 - protein, 375 aa, synthetic construct
FFGGVIGTIALGVATSAQITAAVALVEAKQARSDIEKLKEAIRDTNKAVQCVQSSVGNLICAIKSVQDYVNKEIVPSIARLGCEAAGLQLGIALTQHYSELTNIFGDNIGSLQEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALDPIDISIELNKAKSDLEELKEWIRRSNQKLDSI

SEQ ID NO: 496 - protein, 91 aa, synthetic construct; identical to SEQ ID NO: 307

SEQ ID NO: 497 - protein, 375 aa, synthetic construct
GSGGVIGTIALGVATSAQITAAVALVEAKQARSDIEKLKEAIRDTNKAVQCVQSSVGNLICAIKSVQDYVNKEIVPSIARLGCEAAGLQLGIALTQHYSELTNIFGDNIGSLQEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALDPIDISIELNKLKSDLEELKEWIRRSNQKLDSI

SEQ ID NO: 498 - protein, 91 aa, synthetic construct; identical to SEQ ID NO: 307

SEQ ID NO: 499 - protein, 375 aa, synthetic construct
GSGGVIGTIALGVATSAQITAAVALVEAKQARSDIEKLKEAIRDTNKAVQCVQSSVGNLICAIKSVQDYVNKEIVPSIARLGCEAAGLQLGIALTQHYSCLTNIFGDNIGSLQEKGIKLQGIASCYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALDPIDISIELNKLKSDLEELKEWIRRSNQKLDSI

SEQ ID NO: 500 - protein, 91 aa, synthetic construct; identical to SEQ ID NO: 307

SEQ ID NO: 501 - protein, 375 aa, synthetic construct
GSGGVIGTIALGVATSAQITAAVALVEAKQARSDIEKLKEAIRDTNKAVQCVQSSVGNLICAIKSVQDYVNKEIVPSIARLGCEAAGLQLGIALTQHYSELTNIFGDNIGSLQEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALDPIDISIELNKAKSDLEELKEWIRRSNQKLDSI

SEQ ID NO: 502 - protein, 91 aa, synthetic construct; identical to SEQ ID NO: 307

SEQ ID NO: 503 - DNA, 1617 bp, synthetic construct
  source 1..1617: mol_type = other DNA
atgctgatctccatcctgctgatcatcaccacaatgatcatggccagccactgccagatcgacatcaccaagctgcagcacgtgggcgtgctggtcaatagccctaagggcatgaagatcagccagaacttcgagacacgctacctgatcctgtctctgatccccaagatcgaggacagcaacagctgcggcgaccagcagatcaagcagtacaagcggctgctggacagactgatcatccctctgtacgacggcctgcggctgcagaaagatgtgatcgtgaccaatcaagagagcaacgagaacacagaccccagaaccgagagattcttcggcggcgtgatcggcacaatcgccctgggagttgctacaagcgcccagattacagccgccgtggctctggtggaagccaagcaggccagaagcgacatcgagaagctgaaagaggccatccgggacaccaacaaggccgtgcagtgcgtgcaaagcagcgtgggcaatctgatctgcgccattaagagcgtgcaggactacgtgaacaaagagatcgtcccctctatcgccagactgggatgtgaagctgccggactgcagctgggaattgccctgacacagcactacagctgcctgaccaacatcttcggcgacaacatcggcagcctgcaagagaagggcattaagctgcagggaatcgccagctgctaccgcaccaacatcaccgagatcttcaccaccagcaccgtggataagtacgacatctacgacctgctgttcaccgagagcatcaaagtgcgcgtgatcgacgtggacctgaacgactacagcatcaccctgcaagtgcggctgcctctgctgaccagactgctgaacacccagatctacaaggtggacagcatctcctacaacatccagaaccgcgagtggtacatcccactgcctagccacattatgaccaagggcgcctttctcggcggagccgatgtgaaagagtgcatcgaggccttcagcagctacatctgccctagcgatcctggcttcgtgctgaaccacgagatggaaagctgcctgagcggcaacatctctcagtgccctagaaccaccgtgacctccgacatcgtgcccagatacgcctttgtgaatggcggcgtggtggccaactgcatcaccaccacctgtacctgcaacggcatcggcaaccggatcaaccagcctccagatcagggcgtgaagattatcacccacaaagagtgtaacaccatcggcatcaacggcatgctgttcaataccaacaaagagggcaccctggccttctacacccctgacgatatcaccctgaacaacagcgtggccctggatcctatcgacatctccatcgagctgaacaagctcaagagcgacctggaagaactgaaagagtggatccggcggagcaaccagaagctggatagcatcggaagctggcaccagagcagcaccaccatcatcgtgatcctgattatgatgattatcctgttcatcatcaacattaccatcatcacgatcgccatcaagtactaccggatccagaaacggaaccgcgtggaccagaatgacaagccctacgtgctgacaaacaag

SEQ ID NO: 504 - RNA, 1617 nt, synthetic construct
  source 1..1617: mol_type = other RNA
atgctgatctccatcctgctgatcatcaccacaatgatcatggccagccactgccagatcgacatcaccaagctgcagcacgtgggcgtgctggtcaatagccctaagggcatgaagatcagccagaacttcgagacacgctacctgatcctgtctctgatccccaagatcgaggacagcaacagctgcggcgaccagcagatcaagcagtacaagcggctgctggacagactgatcatccctctgtacgacggcctgcggctgcagaaagatgtgatcgtgaccaatcaagagagcaacgagaacacagaccccagaaccgagagattcttcggcggcgtgatcggcacaatcgccctgggagttgctacaagcgcccagattacagccgccgtggctctggtggaagccaagcaggccagaagcgacatcgagaagctgaaagaggccatccgggacaccaacaaggccgtgcagtgcgtgcaaagcagcgtgggcaatctgatctgcgccattaagagcgtgcaggactacgtgaacaaagagatcgtcccctctatcgccagactgggatgtgaagctgccggactgcagctgggaattgccctgacacagcactacagctgcctgaccaacatcttcggcgacaacatcggcagcctgcaagagaagggcattaagctgcagggaatcgccagctgctaccgcaccaacatcaccgagatcttcaccaccagcaccgtggataagtacgacatctacgacctgctgttcaccgagagcatcaaagtgcgcgtgatcgacgtggacctgaacgactacagcatcaccctgcaagtgcggctgcctctgctgaccagactgctgaacacccagatctacaaggtggacagcatctcctacaacatccagaaccgcgagtggtacatcccactgcctagccacattatgaccaagggcgcctttctcggcggagccgatgtgaaagagtgcatcgaggccttcagcagctacatctgccctagcgatcctggcttcgtgctgaaccacgagatggaaagctgcctgagcggcaacatctctcagtgccctagaaccaccgtgacctccgacatcgtgcccagatacgcctttgtgaatggcggcgtggtggccaactgcatcaccaccacctgtacctgcaacggcatcggcaaccggatcaaccagcctccagatcagggcgtgaagattatcacccacaaagagtgtaacaccatcggcatcaacggcatgctgttcaataccaacaaagagggcaccctggccttctacacccctgacgatatcaccctgaacaacagcgtggccctggatcctatcgacatctccatcgagctgaacaagctcaagagcgacctggaagaactgaaagagtggatccggcggagcaaccagaagctggatagcatcggaagctggcaccagagcagcaccaccatcatcgtgatcctgattatgatgattatcctgttcatcatcaacattaccatcatcacgatcgccatcaagtactaccggatccagaaacggaaccgcgtggaccagaatgacaagccctacgtgctgacaaacaag

SEQ ID NO: 505 - DNA, 1617 bp, synthetic construct
  source 1..1617: mol_type = other DNA
atgctgatctccatcctgctgatcatcaccacaatgatcatggccagccactgccagatcgacatcaccaagctgcagcacgtgggcgtgctggtcaatagccctaagggcatgaagatcagccagaacttcgagacacgctacctgatcctgtctctgatccccaagatcgaggacagcaacagctgcggcgaccagcagatcaagcagtacaagcggctgctggacagactgatcatccctctgtacgacggcctgcggctgcagaaagatgtgatcgtgaccaatcaagagagcaacgagaacacagaccccagaaccgagagattcttcggcggcgtgatcggcacaatcgccctgggagttgctacaagcgcccagattacagccgccgtggctctggtggaagccaagcaggccagaagcgacatcgagaagctgaaagaggccatccgggacaccaacaaggccgtgcagtgcgtgcaaagcagcgtgggcaatctgatctgcgccattaagagcgtgcaggactacgtgaacaaagagatcgtcccctctatcgccagactgggatgtgaagctgccggactgcagctgggaattgccctgacacagcactacagctgcctgaccaacatcttcggcgacaacatcggcagcctgcaagagaagggcattaagctgcagggaatcgccagctgctaccgcaccaacatcaccgagatcttcaccaccagcaccgtggataagtacgacatctacgacctgctgttcaccgagagcatcaaagtgcgcgtgatcgacgtggacctgaacgactacagcatcaccctgcaagtgcggctgcctctgctgaccagactgctgaacacccagatctacaaggtggacagcatctcctacaacatccagaaccgcgagtggtacatcccactgcctagccacattatgaccaagggcgcctttctcggcggagccgatgtgaaagagtgcatcgaggccttcagcagctacatctgccctagcgatcctggcttcgtgctgaaccacgagatggaaagctgcctgagcggcaacatctctcagtgccctagaaccaccgtgacctccgacatcgtgcccagatacgcctttgtgaatggcggcgtggtggccaactgcatcaccaccacctgtacctgcaacggcatcggcaaccggatcaaccagcctccagatcagggcgtgaagattatcacccacaaagagtgtaacaccatcggcatcaacggcatgctgttcaataccaacaaagagggcaccctggccttctacacccctgacgatatcaccctgaacaacagcgtggccctggatcctatcgacatctccatcgagctgaacaagctcaagagcgacctggaagaaagcaaagagtggttccggcggagcaaccagaagctggatagcatcggaagctggcaccagagcagcaccaccatcatcgtgatcctgattatgatgattatcctgttcatcatcaacattaccatcatcacgatcgccatcaagtactaccggatccagaaacggaaccgcgtggaccagaatgacaagccctacgtgctgacaaacaag

SEQ ID NO: 506 - RNA, 1617 nt, synthetic construct
  source 1..1617: mol_type = other RNA
atgctgatctccatcctgctgatcatcaccacaatgatcatggccagccactgccagatcgacatcaccaagctgcagcacgtgggcgtgctggtcaatagccctaagggcatgaagatcagccagaacttcgagacacgctacctgatcctgtctctgatccccaagatcgaggacagcaacagctgcggcgaccagcagatcaagcagtacaagcggctgctggacagactgatcatccctctgtacgacggcctgcggctgcagaaagatgtgatcgtgaccaatcaagagagcaacgagaacacagaccccagaaccgagagattcttcggcggcgtgatcggcacaatcgccctgggagttgctacaagcgcccagattacagccgccgtggctctggtggaagccaagcaggccagaagcgacatcgagaagctgaaagaggccatccgggacaccaacaaggccgtgcagtgcgtgcaaagcagcgtgggcaatctgatctgcgccattaagagcgtgcaggactacgtgaacaaagagatcgtcccctctatcgccagactgggatgtgaagctgccggactgcagctgggaattgccctgacacagcactacagctgcctgaccaacatcttcggcgacaacatcggcagcctgcaagagaagggcattaagctgcagggaatcgccagctgctaccgcaccaacatcaccgagatcttcaccaccagcaccgtggataagtacgacatctacgacctgctgttcaccgagagcatcaaagtgcgcgtgatcgacgtggacctgaacgactacagcatcaccctgcaagtgcggctgcctctgctgaccagactgctgaacacccagatctacaaggtggacagcatctcctacaacatccagaaccgcgagtggtacatcccactgcctagccacattatgaccaagggcgcctttctcggcggagccgatgtgaaagagtgcatcgaggccttcagcagctacatctgccctagcgatcctggcttcgtgctgaaccacgagatggaaagctgcctgagcggcaacatctctcagtgccctagaaccaccgtgacctccgacatcgtgcccagatacgcctttgtgaatggcggcgtggtggccaactgcatcaccaccacctgtacctgcaacggcatcggcaaccggatcaaccagcctccagatcagggcgtgaagattatcacccacaaagagtgtaacaccatcggcatcaacggcatgctgttcaataccaacaaagagggcaccctggccttctacacccctgacgatatcaccctgaacaacagcgtggccctggatcctatcgacatctccatcgagctgaacaagctcaagagcgacctggaagaaagcaaagagtggttccggcggagcaaccagaagctggatagcatcggaagctggcaccagagcagcaccaccatcatcgtgatcctgattatgatgattatcctgttcatcatcaacattaccatcatcacgatcgccatcaagtactaccggatccagaaacggaaccgcgtggaccagaatgacaagccctacgtgctgacaaacaag

SEQ ID NO: 507 - DNA, 1617 bp, synthetic construct
  source 1..1617: mol_type = other DNA
atgctgatctccatcctgctgatcatcaccacaatgatcatggccagccactgccagatcgacatcaccaagctgcagcacgtgggcgtgctggtcaatagccctaagggcatgaagatcagccagaacttcgagacacgctacctgatcctgtctctgatccccaagatcgaggacagcaacagctgcggcgaccagcagatcaagcagtacaagcggctgctggacagactgatcatccctctgtacgacggcctgcggctgcagaaagatgtgatcgtgaccaatcaagagagcaacgagaacacagaccccagaaccgagagaggcagcggcggcgtgatcggcacaatcgccctgggagttgctacaagcgcccagattacagccgccgtggctctggtggaagccaagcaggccagaagcgacatcgagaagctgaaagaggccatccgggacaccaacaaggccgtgcagtgcgtgcaaagcagcgtgggcaatctgatctgcgccattaagagcgtgcaggactacgtgaacaaagagatcgtcccctctatcgccagactgggatgtgaagctgccggactgcagctgggaattgccctgacacagcactacagctgcctgaccaacatcttcggcgacaacatcggcagcctgcaagagaagggcattaagctgcagggaatcgccagctgctaccgcaccaacatcaccgagatcttcaccaccagcaccgtggataagtacgacatctacgacctgctgttcaccgagagcatcaaagtgcgcgtgatcgacgtggacctgaacgactacagcatcaccctgcaagtgcggctgcctctgctgaccagactgctgaacacccagatctacaaggtggacagcatctcctacaacatccagaaccgcgagtggtacatcccactgcctagccacattatgaccaagggcgcctttctcggcggagccgatgtgaaagagtgcatcgaggccttcagcagctacatctgccctagcgatcctggcttcgtgctgaaccacgagatggaaagctgcctgagcggcaacatctctcagtgccctagaaccaccgtgacctccgacatcgtgcccagatacgcctttgtgaatggcggcgtggtggccaactgcatcaccaccacctgtacctgcaacggcatcggcaaccggatcaaccagcctccagatcagggcgtgaagattatcacccacaaagagtgtaacaccatcggcatcaacggcatgctgttcaataccaacaaagagggcaccctggccttctacacccctgacgatatcaccctgaacaacagcgtggccctggatcctatcgacatctccatcgagctgaacaagctcaagagcgacctggaagaactgaaagagtggatccggcggagcaaccagaagctggatagcatcggaagctggcaccagagcagcaccaccatcatcgtgatcctgattatgatgattatcctgttcatcatcaacattaccatcatcacgatcgccatcaagtactaccggatccagaaacggaaccgcgtggaccagaatgacaagccctacgtgctgacaaacaag

SEQ ID NO: 508 - RNA, 1617 nt, synthetic construct
  source 1..1617: mol_type = other RNA
atgctgatctccatcctgctgatcatcaccacaatgatcatggccagccactgccagatcgacatcaccaagctgcagcacgtgggcgtgctggtcaatagccctaagggcatgaagatcagccagaacttcgagacacgctacctgatcctgtctctgatccccaagatcgaggacagcaacagctgcggcgaccagcagatcaagcagtacaagcggctgctggacagactgatcatccctctgtacgacggcctgcggctgcagaaagatgtgatcgtgaccaatcaagagagcaacgagaacacagaccccagaaccgagagaggcagcggcggcgtgatcggcacaatcgccctgggagttgctacaagcgcccagattacagccgccgtggctctggtggaagccaagcaggccagaagcgacatcgagaagctgaaagaggccatccgggacaccaacaaggccgtgcagtgcgtgcaaagcagcgtgggcaatctgatctgcgccattaagagcgtgcaggactacgtgaacaaagagatcgtcccctctatcgccagactgggatgtgaagctgccggactgcagctgggaattgccctgacacagcactacagctgcctgaccaacatcttcggcgacaacatcggcagcctgcaagagaagggcattaagctgcagggaatcgccagctgctaccgcaccaacatcaccgagatcttcaccaccagcaccgtggataagtacgacatctacgacctgctgttcaccgagagcatcaaagtgcgcgtgatcgacgtggacctgaacgactacagcatcaccctgcaagtgcggctgcctctgctgaccagactgctgaacacccagatctacaaggtggacagcatctcctacaacatccagaaccgcgagtggtacatcccactgcctagccacattatgaccaagggcgcctttctcggcggagccgatgtgaaagagtgcatcgaggccttcagcagctacatctgccctagcgatcctggcttcgtgctgaaccacgagatggaaagctgcctgagcggcaacatctctcagtgccctagaaccaccgtgacctccgacatcgtgcccagatacgcctttgtgaatggcggcgtggtggccaactgcatcaccaccacctgtacctgcaacggcatcggcaaccggatcaaccagcctccagatcagggcgtgaagattatcacccacaaagagtgtaacaccatcggcatcaacggcatgctgttcaataccaacaaagagggcaccctggccttctacacccctgacgatatcaccctgaacaacagcgtggccctggatcctatcgacatctccatcgagctgaacaagctcaagagcgacctggaagaactgaaagagtggatccggcggagcaaccagaagctggatagcatcggaagctggcaccagagcagcaccaccatcatcgtgatcctgattatgatgattatcctgttcatcatcaacattaccatcatcacgatcgccatcaagtactaccggatccagaaacggaaccgcgtggaccagaatgacaagccctacgtgctgacaaacaag

SEQ ID NO: 509 - DNA, 1617 bp, synthetic construct
  source 1..1617: mol_type = other DNA
atgctgatctccatcctgctgatcatcaccacaatgatcatggccagccactgccagatcgacatcaccaagctgcagcacgtgggcgtgctggtcaatagccctaagggcatgaagatcagccagaacttcgagacacgctacctgatcctgtctctgatccccaagatcgaggacagcaacagctgcggcgaccagcagatcaagcagtacaagcggctgctggacagactgatcatccctctgtacgacggcctgcggctgcagaaagatgtgatcgtgaccaatcaagagagcaacgagaacacagaccccagaaccgagagattcttcggcggcgtgatcggcacaatcgccctgggagttgctacaagcgcccagattacagccgccgtggctctggtggaagccaagcaggccagaagcgacatcgagaagctgaaagaggccatccgggacaccaacaaggccgtgcagtgcgtgcaaagcagcgtgggcaatctgatctgcgccattaagagcgtgcaggactacgtgaacaaagagatcgtcccctctatcgccagactgggatgtgaagctgccggactgcagctgggaattgccctgacacagcactacagcgagctgaccaacatcttcggcgacaacatcggcagcctgcaagagaagggcattaagctgcagggaatcgccagcctgtaccgcaccaacatcaccgagatcttcaccaccagcaccgtggataagtacgacatctacgacctgctgttcaccgagagcatcaaagtgcgcgtgatcgacgtggacctgaacgactacagcatcaccctgcaagtgcggctgcctctgctgaccagactgctgaacacccagatctacaaggtggacagcatctcctacaacatccagaaccgcgagtggtacatcccactgcctagccacattatgaccaagggcgcctttctcggcggagccgatgtgaaagagtgcatcgaggccttcagcagctacatctgccctagcgatcctggcttcgtgctgaaccacgagatggaaagctgcctgagcggcaacatctctcagtgccctagaaccaccgtgacctccgacatcgtgcccagatacgcctttgtgaatggcggcgtggtggccaactgcatcaccaccacctgtacctgcaacggcatcggcaaccggatcaaccagcctccagatcagggcgtgaagattatcacccacaaagagtgtaacaccatcggcatcaacggcatgctgttcaataccaacaaagagggcaccctggccttctacacccctgacgatatcaccctgaacaacagcgtggccctggatcctatcgacatctccatcgagctgaacaagctcaagagcgacctggaagaactgaaagagtggatccggcggagcaaccagaagctggatagcatcggaagctggcaccagagcagcaccaccatcatcgtgatcctgattatgatgattatcctgttcatcatcaacattaccatcatcacgatcgccatcaagtactaccggatccagaaacggaaccgcgtggaccagaatgacaagccctacgtgctgacaaacaag

SEQ ID NO: 510 - RNA, 1617 nt, synthetic construct
  source 1..1617: mol_type = other RNA
atgctgatctccatcctgctgatcatcaccacaatgatcatggccagccactgccagatcgacatcaccaagctgcagcacgtgggcgtgctggtcaatagccctaagggcatgaagatcagccagaacttcgagacacgctacctgatcctgtctctgatccccaagatcgaggacagcaacagctgcggcgaccagcagatcaagcagtacaagcggctgctggacagactgatcatccctctgtacgacggcctgcggctgcagaaagatgtgatcgtgaccaatcaagagagcaacgagaacacagaccccagaaccgagagattcttcggcggcgtgatcggcacaatcgccctgggagttgctacaagcgcccagattacagccgccgtggctctggtggaagccaagcaggccagaagcgacatcgagaagctgaaagaggccatccgggacaccaacaaggccgtgcagtgcgtgcaaagcagcgtgggcaatctgatctgcgccattaagagcgtgcaggactacgtgaacaaagagatcgtcccctctatcgccagactgggatgtgaagctgccggactgcagctgggaattgccctgacacagcactacagcgagctgaccaacatcttcggcgacaacatcggcagcctgcaagagaagggcattaagctgcagggaatcgccagcctgtaccgcaccaacatcaccgagatcttcaccaccagcaccgtggataagtacgacatctacgacctgctgttcaccgagagcatcaaagtgcgcgtgatcgacgtggacctgaacgactacagcatcaccctgcaagtgcggctgcctctgctgaccagactgctgaacacccagatctacaaggtggacagcatctcctacaacatccagaaccgcgagtggtacatcccactgcctagccacattatgaccaagggcgcctttctcggcggagccgatgtgaaagagtgcatcgaggccttcagcagctacatctgccctagcgatcctggcttcgtgctgaaccacgagatggaaagctgcctgagcggcaacatctctcagtgccctagaaccaccgtgacctccgacatcgtgcccagatacgcctttgtgaatggcggcgtggtggccaactgcatcaccaccacctgtacctgcaacggcatcggcaaccggatcaaccagcctccagatcagggcgtgaagattatcacccacaaagagtgtaacaccatcggcatcaacggcatgctgttcaataccaacaaagagggcaccctggccttctacacccctgacgatatcaccctgaacaacagcgtggccctggatcctatcgacatctccatcgagctgaacaagctcaagagcgacctggaagaactgaaagagtggatccggcggagcaaccagaagctggatagcatcggaagctggcaccagagcagcaccaccatcatcgtgatcctgattatgatgattatcctgttcatcatcaacattaccatcatcacgatcgccatcaagtactaccggatccagaaacggaaccgcgtggaccagaatgacaagccctacgtgctgacaaacaag

SEQ ID NO: 511 - DNA, 1617 bp, synthetic construct
  source 1..1617: mol_type = other DNA
atgagctggaaagtcatgatcatcatcagcctgctgatcacccctcagcacggcctgaaagagagctacctggaagaaagctgcagcaccatcaccgagggctacctgagcgtgctgagaaccggctggtacaccaacgtgttcaccctggaagtgggcgacgtggaaaacctgacctgcacagatggccccagccccatcaagaccgagctggatctgacaaagagcgccctgcgcgagctgaaaaccgtgtctgcagatcagctggccagagaggaacagatcgagaaccccagacagagcagattcgtgctgggagctatcgccctgggagttgctacagctgctgctgtgacagccggaatcgccattgccaagaccatccggctggaaagcgaagtgaacgccatcaagggcgcactgaaaaccaccaacgaggccgtgtctaccctcggcaacggtgttagagtgctggccacagccgtgcgggaactgaaagaattcgtgtccaagaacctgaccagcgccatcaacaagaacaagtgcgacattgccgacctgaagatggccgtgtccttcagccagttcaaccggcggttcctgaatgtcgtgcggcagttctctgacaacgccggcatcacaccagccattagcctggacctgatgaacgacgccgaactggctagagccgtgtcttacatgcctacctctgccggccagatcaagctgatgctggaaaacagagccatggtccgacggaaaggcttcggcatcctgatcggcgtgtacggcagcagcgtgatctacatggtgcagctgcctatcttcggcgtgatcaacaccccttgctggatcatcaaggccgctcctagctgcagcgagaaggacggcaattacgcctgcctgctgagagaggaccaaggctggtactgcaagaatgccggcagcaccgtgtactaccccaacgagaaggattgcgagacacggggcgatcacgtgttctgtgatacagccgccggaatcaacgtggccgagcagagcagagagtgcaacatcaacatcagcaccacaaactacccctgcaaggtgtccaccggcagacaccctatcagcatggtggctctgtctccactgggagccctggtggcttgttataagggcgtgtcctgtagcatcggcagcaatcaagtgggcatcatcaagcagctgcccaagggctgctcctacatcaccaatcaggacgccgacaccgtgaccatcgacaataccgtgtatcagctgagcaaggtggaaggcgaacagcacgtgatcaagggcagacctgtgtccaacagcttcgaccccatcagattccccgaggaccagttcaatgtggccctggaccaggtgttcgagagcatcgagaatagccaggctctggtggaccagtccaacaagatcctgaactccgccgagaagggcaacaccggcttcatcatcgtgatcatcctgattgccgtgctgggcctgaccatgatcagcgtgtccatcatcattatcatcaagaaaacgcggaagcccgccggcgcccctccagaacttaatggcgtgaccaacggcggcttcattccccactct

SEQ ID NO: 512 - RNA, 1617 nt, synthetic construct
  source 1..1617: mol_type = other RNA
atgagctggaaagtcatgatcatcatcagcctgctgatcacccctcagcacggcctgaaagagagctacctggaagaaagctgcagcaccatcaccgagggctacctgagcgtgctgagaaccggctggtacaccaacgtgttcaccctggaagtgggcgacgtggaaaacctgacctgcacagatggccccagccccatcaagaccgagctggatctgacaaagagcgccctgcgcgagctgaaaaccgtgtctgcagatcagctggccagagaggaacagatcgagaaccccagacagagcagattcgtgctgggagctatcgccctgggagttgctacagctgctgctgtgacagccggaatcgccattgccaagaccatccggctggaaagcgaagtgaacgccatcaagggcgcactgaaaaccaccaacgaggccgtgtctaccctcggcaacggtgttagagtgctggccacagccgtgcgggaactgaaagaattcgtgtccaagaacctgaccagcgccatcaacaagaacaagtgcgacattgccgacctgaagatggccgtgtccttcagccagttcaaccggcggttcctgaatgtcgtgcggcagttctctgacaacgccggcatcacaccagccattagcctggacctgatgaacgacgccgaactggctagagccgtgtcttacatgcctacctctgccggccagatcaagctgatgctggaaaacagagccatggtccgacggaaaggcttcggcatcctgatcggcgtgtacggcagcagcgtgatctacatggtgcagctgcctatcttcggcgtgatcaacaccccttgctggatcatcaaggccgctcctagctgcagcgagaaggacggcaattacgcctgcctgctgagagaggaccaaggctggtactgcaagaatgccggcagcaccgtgtactaccccaacgagaaggattgcgagacacggggcgatcacgtgttctgtgatacagccgccggaatcaacgtggccgagcagagcagagagtgcaacatcaacatcagcaccacaaactacccctgcaaggtgtccaccggcagacaccctatcagcatggtggctctgtctccactgggagccctggtggcttgttataagggcgtgtcctgtagcatcggcagcaatcaagtgggcatcatcaagcagctgcccaagggctgctcctacatcaccaatcaggacgccgacaccgtgaccatcgacaataccgtgtatcagctgagcaaggtggaaggcgaacagcacgtgatcaagggcagacctgtgtccaacagcttcgaccccatcagattccccgaggaccagttcaatgtggccctggaccaggtgttcgagagcatcgagaatagccaggctctggtggaccagtccaacaagatcctgaactccgccgagaagggcaacaccggcttcatcatcgtgatcatcctgattgccgtgctgggcctgaccatgatcagcgtgtccatcatcattatcatcaagaaaacgcggaagcccgccggcgcccctccagaacttaatggcgtgaccaacggcggcttcattccccactct

SEQ ID NO: 513 - DNA, 1617 bp, synthetic construct
  source 1..1617: mol_type = other DNA
atgagctggaaagtcatgatcatcatcagcctgctgatcacccctcagcacggcctgaaagagagctacctggaagaaagctgcagcaccatcaccgagggctacctgagcgtgctgagaaccggctggtacaccaacgtgttcaccctggaagtgggcgacgtggaaaacctgacctgcacagatggccccagcctgatcaagaccgagctggatctgacaaagagcgccctgcgcgagctgaaaaccgtgtctgcagatcagctggccagagaggaacagatcgagaaccccagacagagcagattcgtgctgggagctatcgccctgggagttgctacagctgctgctgtgacagccggaatcgccattgccaagaccatccggctggaaagcgaagtgaacgccatcaagggcgcactgaaaaccaccaacgaggccgtgtctaccctcggcaacggtgttagagtgctggccacagccgtgcgggaactgaaagaattcgtgtccaagaacctgaccagcgccatcaacaagaacaagtgcgacattgccgaccccaagatggccgtgtccttcagccagttcaaccggcggttcctgaatgtcgtgcggcagttctctgacaacgccggcatcacaccagccattagcctggacctgatgaacgacgccgaactggctagagccgtgtcttacatgcctacctctgccggccagatcaagctgatgctggaaaacagagccatggtccgacggaaaggcttcggcatcctgatcggcgtgtacggcagcagcgtgatctacatggtgcagctgcctatcttcggcgtgatcaacaccccttgctggatcatcaaggccgctcctagctgcagcgagaaggacggcaattacgcctgcctgctgagagaggaccaaggctggtactgcaagaatgccggcagcaccgtgtactaccccaacgagaaggattgcgagacacggggcgatcacgtgttctgtgatacagccgccggaatcaacgtggccgagcagagcagagagtgcaacatcaacatcagcaccacaaactacccctgcaaggtgtccaccggcagacaccctatcagcatggtggctctgtctccactgggagccctggtggcttgttataagggcgtgtcctgtagcatcggcagcaatcaagtgggcatcatcaagcagctgcccaagggctgctcctacatcaccaatcaggacgccgacaccgtgaccatcgacaataccgtgtatcagctgagcaaggtggaaggcgaacagcacgtgatcaagggcagacctgtgtccaacagcttcgaccccatcagattccccgaggaccagttcaatgtggccctggaccaggtgttcgagagcatcgagaatagccaggctctggtggaccagtccaacaagatcctgaactccgccgagaagggcaacaccggcttcatcatcgtgatcatcctgattgccgtgctgggcctgaccatgatcagcgtgtccatcatcattatcatcaagaaaacgcggaagcccgccggcgcccctccagaacttaatggcgtgaccaacggcggcttcattccccactct

SEQ ID NO: 514 - RNA, 1617 nt, synthetic construct
  source 1..1617: mol_type = other RNA
atgagctggaaagtcatgatcatcatcagcctgctgatcacccctcagcacggcctgaaagagagctacctggaagaaagctgcagcaccatcaccgagggctacctgagcgtgctgagaaccggctggtacaccaacgtgttcaccctggaagtgggcgacgtggaaaacctgacctgcacagatggccccagcctgatcaagaccgagctggatctgacaaagagcgccctgcgcgagctgaaaaccgtgtctgcagatcagctggccagagaggaacagatcgagaaccccagacagagcagattcgtgctgggagctatcgccctgggagttgctacagctgctgctgtgacagccggaatcgccattgccaagaccatccggctggaaagcgaagtgaacgccatcaagggcgcactgaaaaccaccaacgaggccgtgtctaccctcggcaacggtgttagagtgctggccacagccgtgcgggaactgaaagaattcgtgtccaagaacctgaccagcgccatcaacaagaacaagtgcgacattgccgaccccaagatggccgtgtccttcagccagttcaaccggcggttcctgaatgtcgtgcggcagttctctgacaacgccggcatcacaccagccattagcctggacctgatgaacgacgccgaactggctagagccgtgtcttacatgcctacctctgccggccagatcaagctgatgctggaaaacagagccatggtccgacggaaaggcttcggcatcctgatcggcgtgtacggcagcagcgtgatctacatggtgcagctgcctatcttcggcgtgatcaacaccccttgctggatcatcaaggccgctcctagctgcagcgagaaggacggcaattacgcctgcctgctgagagaggaccaaggctggtactgcaagaatgccggcagcaccgtgtactaccccaacgagaaggattgcgagacacggggcgatcacgtgttctgtgatacagccgccggaatcaacgtggccgagcagagcagagagtgcaacatcaacatcagcaccacaaactacccctgcaaggtgtccaccggcagacaccctatcagcatggtggctctgtctccactgggagccctggtggcttgttataagggcgtgtcctgtagcatcggcagcaatcaagtgggcatcatcaagcagctgcccaagggctgctcctacatcaccaatcaggacgccgacaccgtgaccatcgacaataccgtgtatcagctgagcaaggtggaaggcgaacagcacgtgatcaagggcagacctgtgtccaacagcttcgaccccatcagattccccgaggaccagttcaatgtggccctggaccaggtgttcgagagcatcgagaatagccaggctctggtggaccagtccaacaagatcctgaactccgccgagaagggcaacaccggcttcatcatcgtgatcatcctgattgccgtgctgggcctgaccatgatcagcgtgtccatcatcattatcatcaagaaaacgcggaagcccgccggcgcccctccagaacttaatggcgtgaccaacggcggcttcattccccactct

SEQ ID NO: 515 - DNA, 1617 bp, synthetic construct
  source 1..1617: mol_type = other DNA
atgagctggaaagtcatgatcatcatcagcctgctgatcacccctcagcacggcctgaaagagagctacctggaagaaagctgcagcaccatcaccgagggctacctgagcgtgctgagaaccggctggtacaccaacgtgttcaccctggaagtgggcgacgtggaaaacctgacctgcacagatggccccagcctgatcaagaccgagctggatctgacaaagagcgccctgcgcgagctgaaaaccgtgtctgcagatcagctggccagagaggaacagatcgagaaccccagacggcggagattcgtgctgggagctatcgccctgggagttgctacagctgctgctgtgacagccggaatcgccattgccaagaccatccggctggaaagcgaagtgaacgccatcaagggcgcactgaaaaccaccaacgaggccgtgtctaccctcggcaacggtgttagagtgctggccacagccgtgcgggaactgaaagaattcgtgtccaagaacctgaccagcgccatcaacaagaacaagtgcgacattgccgaccccaagatggccgtgtccttcagccagttcaaccggcggttcctgaatgtcgtgcggcagttctctgacaacgccggcatcacaccagccattagcctggacctgatgaacgacgccgaactggctagagccgtgtcttacatgcctacctctgccggccagatcaagctgatgctggaaaacagagccatggtccgacggaaaggcttcggcatcctgatcggcgtgtacggcagcagcgtgatctacatggtgcagctgcctatcttcggcgtgatcaacaccccttgctggatcatcaaggccgctcctagctgcagcgagaaggacggcaattacgcctgcctgctgagagaggaccaaggctggtactgcaagaatgccggcagcaccgtgtactaccccaacgagaaggattgcgagacacggggcgatcacgtgttctgtgatacagccgccggaatcaacgtggccgagcagagcagagagtgcaacatcaacatcagcaccacaaactacccctgcaaggtgtccaccggcagacaccctatcagcatggtggctctgtctccactgggagccctggtggcttgttataagggcgtgtcctgtagcatcggcagcaatcaagtgggcatcatcaagcagctgcccaagggctgctcctacatcaccaatcaggacgccgacaccgtgaccatcgacaataccgtgtatcagctgagcaaggtggaaggcgaacagcacgtgatcaagggcagacctgtgtccaacagcttcgaccccatcagattccccgaggaccagttcaatgtggccctggaccaggtgttcgagagcatcgagaatagccaggctctggtggaccagtccaacaagatcctgaactccgccgagaagggcaacaccggcttcatcatcgtgatcatcctgattgccgtgctgggcctgaccatgatcagcgtgtccatcatcattatcatcaagaaaacgcggaagcccgccggcgcccctccagaacttaatggcgtgaccaacggcggcttcattccccactct

SEQ ID NO: 516 - RNA, 1617 nt, synthetic construct
  source 1..1617: mol_type = other RNA
atgagctggaaagtcatgatcatcatcagcctgctgatcacccctcagcacggcctgaaagagagctacctggaagaaagctgcagcaccatcaccgagggctacctgagcgtgctgagaaccggctggtacaccaacgtgttcaccctggaagtgggcgacgtggaaaacctgacctgcacagatggccccagcctgatcaagaccgagctggatctgacaaagagcgccctgcgcgagctgaaaaccgtgtctgcagatcagctggccagagaggaacagatcgagaaccccagacggcggagattcgtgctgggagctatcgccctgggagttgctacagctgctgctgtgacagccggaatcgccattgccaagaccatccggctggaaagcgaagtgaacgccatcaagggcgcactgaaaaccaccaacgaggccgtgtctaccctcggcaacggtgttagagtgctggccacagccgtgcgggaactgaaagaattcgtgtccaagaacctgaccagcgccatcaacaagaacaagtgcgacattgccgaccccaagatggccgtgtccttcagccagttcaaccggcggttcctgaatgtcgtgcggcagttctctgacaacgccggcatcacaccagccattagcctggacctgatgaacgacgccgaactggctagagccgtgtcttacatgcctacctctgccggccagatcaagctgatgctggaaaacagagccatggtccgacggaaaggcttcggcatcctgatcggcgtgtacggcagcagcgtgatctacatggtgcagctgcctatcttcggcgtgatcaacaccccttgctggatcatcaaggccgctcctagctgcagcgagaaggacggcaattacgcctgcctgctgagagaggaccaaggctggtactgcaagaatgccggcagcaccgtgtactaccccaacgagaaggattgcgagacacggggcgatcacgtgttctgtgatacagccgccggaatcaacgtggccgagcagagcagagagtgcaacatcaacatcagcaccacaaactacccctgcaaggtgtccaccggcagacaccctatcagcatggtggctctgtctccactgggagccctggtggcttgttataagggcgtgtcctgtagcatcggcagcaatcaagtgggcatcatcaagcagctgcccaagggctgctcctacatcaccaatcaggacgccgacaccgtgaccatcgacaataccgtgtatcagctgagcaaggtggaaggcgaacagcacgtgatcaagggcagacctgtgtccaacagcttcgaccccatcagattccccgaggaccagttcaatgtggccctggaccaggtgttcgagagcatcgagaatagccaggctctggtggaccagtccaacaagatcctgaactccgccgagaagggcaacaccggcttcatcatcgtgatcatcctgattgccgtgctgggcctgaccatgatcagcgtgtccatcatcattatcatcaagaaaacgcggaagcccgccggcgcccctccagaacttaatggcgtgaccaacggcggcttcattccccactct

SEQ ID NO: 517 - DNA, 1617 bp, synthetic construct
  source 1..1617: mol_type = other DNA
atgagctggaaagtcatgatcatcatcagcctgctgatcacccctcagcacggcctgaaagagagctacctggaagaaagctgcagcaccatcaccgagggctacctgagcgtgctgagaaccggctggtacaccaacgtgttcaccctggaagtgggcgacgtggaaaacctgacctgcacagatggccccagcctgatcaagaccgagctggatctgacaaagagcgccctgcgcgagctgaaaaccgtgtctgcagatcagctggccagagaggaacagatcgagaaccccagacagagcagattcgtgctgggagctatcgccctgggagttgctacagctgctgctgtgacagccggaatcgccattgccaagaccatccggctggaaagcgaagtgaacgccatcaagggctgcctgaaaaccaccaacgaggccgtgtgcaccctcggcaacggtgttagagtgctggccacagccgtgcgggaactgaaagaattcgtgtccaagaacctgaccagcgccatcaacaagaacaagtgcgacattgccgaccccaagatggccgtgtccttcagccagttcaaccggcggttcctgaatgtcgtgcggcagttctctgacaacgccggcatcacaccagccattagcctggacctgatgaacgacgccgaactggctagagccgtgtcttacatgcctacctctgccggccagatcaagctgatgctggaaaacagagccatggtccgacggaaaggcttcggcatcctgatcggcgtgtacggcagcagcgtgatctacatggtgcagctgcctatcttcggcgtgatcaacaccccttgctggatcatcaaggccgctcctagctgcagcgagaaggacggcaattacgcctgcctgctgagagaggaccaaggctggtactgcaagaatgccggcagcaccgtgtactaccccaacgagaaggattgcgagacacggggcgatcacgtgttctgtgatacagccgccggaatcaacgtggccgagcagagcagagagtgcaacatcaacatcagcaccacaaactacccctgcaaggtgtccaccggcagacaccctatcagcatggtggctctgtctccactgggagccctggtggcttgttataagggcgtgtcctgtagcatcggcagcaatcaagtgggcatcatcaagcagctgcccaagggctgctcctacatcaccaatcaggacgccgacaccgtgaccatcgacaataccgtgtatcagctgagcaaggtggaaggcgaacagcacgtgatcaagggcagacctgtgtccaacagcttcgaccccatcagattccccgaggaccagttcaatgtggccctggaccaggtgttcgagagcatcgagaatagccaggctctggtggaccagtccaacaagatcctgaactccgccgagaagggcaacaccggcttcatcatcgtgatcatcctgattgccgtgctgggcctgaccatgatcagcgtgtccatcatcattatcatcaagaaaacgcggaagcccgccggcgcccctccagaacttaatggcgtgaccaacggcggcttcattccccactct

SEQ ID NO: 518 - RNA, 1617 nt, synthetic construct
  source 1..1617: mol_type = other RNA
atgagctggaaagtcatgatcatcatcagcctgctgatcacccctcagcacggcctgaaagagagctacctggaagaaagctgcagcaccatcaccgagggctacctgagcgtgctgagaaccggctggtacaccaacgtgttcaccctggaagtgggcgacgtggaaaacctgacctgcacagatggccccagcctgatcaagaccgagctggatctgacaaagagcgccctgcgcgagctgaaaaccgtgtctgcagatcagctggccagagaggaacagatcgagaaccccagacagagcagattcgtgctgggagctatcgccctgggagttgctacagctgctgctgtgacagccggaatcgccattgccaagaccatccggctggaaagcgaagtgaacgccatcaagggctgcctgaaaaccaccaacgaggccgtgtgcaccctcggcaacggtgttagagtgctggccacagccgtgcgggaactgaaagaattcgtgtccaagaacctgaccagcgccatcaacaagaacaagtgcgacattgccgaccccaagatggccgtgtccttcagccagttcaaccggcggttcctgaatgtcgtgcggcagttctctgacaacgccggcatcacaccagccattagcctggacctgatgaacgacgccgaactggctagagccgtgtcttacatgcctacctctgccggccagatcaagctgatgctggaaaacagagccatggtccgacggaaaggcttcggcatcctgatcggcgtgtacggcagcagcgtgatctacatggtgcagctgcctatcttcggcgtgatcaacaccccttgctggatcatcaaggccgctcctagctgcagcgagaaggacggcaattacgcctgcctgctgagagaggaccaaggctggtactgcaagaatgccggcagcaccgtgtactaccccaacgagaaggattgcgagacacggggcgatcacgtgttctgtgatacagccgccggaatcaacgtggccgagcagagcagagagtgcaacatcaacatcagcaccacaaactacccctgcaaggtgtccaccggcagacaccctatcagcatggtggctctgtctccactgggagccctggtggcttgttataagggcgtgtcctgtagcatcggcagcaatcaagtgggcatcatcaagcagctgcccaagggctgctcctacatcaccaatcaggacgccgacaccgtgaccatcgacaataccgtgtatcagctgagcaaggtggaaggcgaacagcacgtgatcaagggcagacctgtgtccaacagcttcgaccccatcagattccccgaggaccagttcaatgtggccctggaccaggtgttcgagagcatcgagaatagccaggctctggtggaccagtccaacaagatcctgaactccgccgagaagggcaacaccggcttcatcatcgtgatcatcctgattgccgtgctgggcctgaccatgatcagcgtgtccatcatcattatcatcaagaaaacgcggaagcccgccggcgcccctccagaacttaatggcgtgaccaacggcggcttcattccccactct

SEQ ID NO: 519 - DNA, 1617 bp, synthetic construct
  source 1..1617: mol_type = other DNA
atgagctggaaagtcatgatcatcatcagcctgctgatcacccctcagcacggcctgaaagagagctacctggaagaaagctgcagcaccatcaccgagggctacctgagcgtgctgagaaccggctggtacaccaacgtgttcaccctggaagtgggcgacgtggaaaacctgacctgcacagatggccccagcctgatcaagaccgagctggatctgacaaagagcgccctgcgcgagctgaaaaccgtgtctgcagatcagctggccagagaggaacagatcgagaaccccagacggcggagattcgtgctgggagctatcgccctgggagttgctacagctgctgctgtgacagccggaatcgccattgccaagaccatccggctggaaagcgaagtgaacgccatcaagggctgcctgaaaaccaccaacgaggccgtgtgcaccctcggcaacggtgttagagtgctggccacagccgtgcgggaactgaaagaattcgtgtccaagaacctgaccagcgccatcaacaagaacaagtgcgacattgccgaccccaagatggccgtgtccttcagccagttcaaccggcggttcctgaatgtcgtgcggcagttctctgacaacgccggcatcacaccagccattagcctggacctgatgaacgacgccgaactggctagagccgtgtcttacatgcctacctctgccggccagatcaagctgatgctggaaaacagagccatggtccgacggaaaggcttcggcatcctgatcggcgtgtacggcagcagcgtgatctacatggtgcagctgcctatcttcggcgtgatcaacaccccttgctggatcatcaaggccgctcctagctgcagcgagaaggacggcaattacgcctgcctgctgagagaggaccaaggctggtactgcaagaatgccggcagcaccgtgtactaccccaacgagaaggattgcgagacacggggcgatcacgtgttctgtgatacagccgccggaatcaacgtggccgagcagagcagagagtgcaacatcaacatcagcaccacaaactacccctgcaaggtgtccaccggcagacaccctatcagcatggtggctctgtctccactgggagccctggtggcttgttataagggcgtgtcctgtagcatcggcagcaatcaagtgggcatcatcaagcagctgcccaagggctgctcctacatcaccaatcaggacgccgacaccgtgaccatcgacaataccgtgtatcagctgagcaaggtggaaggcgaacagcacgtgatcaagggcagacctgtgtccaacagcttcgaccccatcagattccccgaggaccagttcaatgtggccctggaccaggtgttcgagagcatcgagaatagccaggctctggtggaccagtccaacaagatcctgaactccgccgagaagggcaacaccggcttcatcatcgtgatcatcctgattgccgtgctgggcctgaccatgatcagcgtgtccatcatcattatcatcaagaaaacgcggaagcccgccggcgcccctccagaacttaatggcgtgaccaacggcggcttcattccccactct

SEQ ID NO: 520 - RNA, 1617 nt, synthetic construct
  source 1..1617: mol_type = other RNA
atgagctggaaagtcatgatcatcatcagcctgctgatcacccctcagcacggcctgaaagagagctacctggaagaaagctgcagcaccatcaccgagggctacctgagcgtgctgagaaccggctggtacaccaacgtgttcaccctggaagtgggcgacgtggaaaacctgacctgcacagatggccccagcctgatcaagaccgagctggatctgacaaagagcgccctgcgcgagctgaaaaccgtgtctgcagatcagctggccagagaggaacagatcgagaaccccagacggcggagattcgtgctgggagctatcgccctgggagttgctacagctgctgctgtgacagccggaatcgccattgccaagaccatccggctggaaagcgaagtgaacgccatcaagggctgcctgaaaaccaccaacgaggccgtgtgcaccctcggcaacggtgttagagtgctggccacagccgtgcgggaactgaaagaattcgtgtccaagaacctgaccagcgccatcaacaagaacaagtgcgacattgccgaccccaagatggccgtgtccttcagccagttcaaccggcggttcctgaatgtcgtgcggcagttctctgacaacgccggcatcacaccagccattagcctggacctgatgaacgacgccgaactggctagagccgtgtcttacatgcctacctctgccggccagatcaagctgatgctggaaaacagagccatggtccgacggaaaggcttcggcatcctgatcggcgtgtacggcagcagcgtgatctacatggtgcagctgcctatcttcggcgtgatcaacaccccttgctggatcatcaaggccgctcctagctgcagcgagaaggacggcaattacgcctgcctgctgagagaggaccaaggctggtactgcaagaatgccggcagcaccgtgtactaccccaacgagaaggattgcgagacacggggcgatcacgtgttctgtgatacagccgccggaatcaacgtggccgagcagagcagagagtgcaacatcaacatcagcaccacaaactacccctgcaaggtgtccaccggcagacaccctatcagcatggtggctctgtctccactgggagccctggtggcttgttataagggcgtgtcctgtagcatcggcagcaatcaagtgggcatcatcaagcagctgcccaagggctgctcctacatcaccaatcaggacgccgacaccgtgaccatcgacaataccgtgtatcagctgagcaaggtggaaggcgaacagcacgtgatcaagggcagacctgtgtccaacagcttcgaccccatcagattccccgaggaccagttcaatgtggccctggaccaggtgttcgagagcatcgagaatagccaggctctggtggaccagtccaacaagatcctgaactccgccgagaagggcaacaccggcttcatcatcgtgatcatcctgattgccgtgctgggcctgaccatgatcagcgtgtccatcatcattatcatcaagaaaacgcggaagcccgccggcgcccctccagaacttaatggcgtgaccaacggcggcttcattccccactct

SEQ ID NO: 521 - DNA, 1617 bp, synthetic construct
  source 1..1617: mol_type = other DNA
atgagctggaaagtcatgatcatcatcagcctgctgatcacccctcagcacggcctgaaagagagctacctggaagaaagctgcagcaccatcaccgagggctacctgagcgtgctgagaaccggctggtacaccaacgtgttcatcctggaagtgggcgacgtggaaaacctgacctgcacagatggccccagcctgatcaagaccgagctggatctgacaaagagcgccctgcgcgagctgaaaaccgtgtctgcagatcagctggccagagaggaacagatcgagaaccccagacagagcagattcgtgctgggagctatcgccctgggagttgctacagctgctgctgtgacagccggaatcgccattgccaagaccatccggctggaaagcgaagtgaacgccatcaagggcgcactgaaaaccaccaacgaggccgtgtctaccctcggcaacggtgttagagtgctggccacagccgtgcgggaactgaaagaattcgtgtccaagaacctgaccagcgccatcaacaagaacaagtgcgacattgccgacctgaagatggccgtgtccttcagccagttcaaccggcggttcctgaatgtcgtgcggcagttctctgacaacgccggcatcacaccagccattagcctggacctgatgaacgacgccgaactggctagagccgtgtcttacatgcctacctctgccggccagatcaagctgatgctggaaaacagagccatggtccgacggaaaggcttcggcatcctgatcggcgtgtacggcagcagcgtgatctacatggtgcagctgcctatcttcggcgtgatcaacaccccttgctggatcatcaaggccgctcctagctgcagcgagaaggacggcaattacgcctgcctgctgagagaggaccaaggctggtactgcaagaatgccggcagcaccgtgtactaccccaacgagaaggattgcgagacacggggcgatcacgtgttctgtgatacagccgccggaatcaacgtggccgagcagagcagagagtgcaacatcaacatcagcaccacaaactacccctgcaaggtgtccaccggcagacaccctatcagcatggtggctctgtctccactgggagccctggtggcttgttataagggcgtgtcctgtagcatcggcagcaatcaagtgggcatcatcaagcagctgcccaagggctgctcctacatcaccaatcaggacgccgacaccgtgaccatcgacaataccgtgtatcagctgagcaaggtggaaggcgaacagcacgtgatcaagggcagacctgtgtccaacagcttcgaccccatcagattccccgaggaccagttcaatgtggccctggaccaggtgttcgagagcatcgagaatagccaggctctggtggaccagtccaacaagatcctgaactccgccgagaagggcaacaccggcttcatcatcgtgatcatcctgattgccgtgctgggcctgaccatgatcagcgtgtccatcatcattatcatcaagaaaacgcggaagcccgccggcgcccctccagaacttaatggcgtgaccaacggcggcttcattccccactct

SEQ ID NO: 522 - RNA, 1617 nt, synthetic construct
  source 1..1617: mol_type = other RNA
atgagctggaaagtcatgatcatcatcagcctgctgatcacccctcagcacggcctgaaagagagctacctggaagaaagctgcagcaccatcaccgagggctacctgagcgtgctgagaaccggctggtacaccaacgtgttcatcctggaagtgggcgacgtggaaaacctgacctgcacagatggccccagcctgatcaagaccgagctggatctgacaaagagcgccctgcgcgagctgaaaaccgtgtctgcagatcagctggccagagaggaacagatcgagaaccccagacagagcagattcgtgctgggagctatcgccctgggagttgctacagctgctgctgtgacagccggaatcgccattgccaagaccatccggctggaaagcgaagtgaacgccatcaagggcgcactgaaaaccaccaacgaggccgtgtctaccctcggcaacggtgttagagtgctggccacagccgtgcgggaactgaaagaattcgtgtccaagaacctgaccagcgccatcaacaagaacaagtgcgacattgccgacctgaagatggccgtgtccttcagccagttcaaccggcggttcctgaatgtcgtgcggcagttctctgacaacgccggcatcacaccagccattagcctggacctgatgaacgacgccgaactggctagagccgtgtcttacatgcctacctctgccggccagatcaagctgatgctggaaaacagagccatggtccgacggaaaggcttcggcatcctgatcggcgtgtacggcagcagcgtgatctacatggtgcagctgcctatcttcggcgtgatcaacaccccttgctggatcatcaaggccgctcctagctgcagcgagaaggacggcaattacgcctgcctgctgagagaggaccaaggctggtactgcaagaatgccggcagcaccgtgtactaccccaacgagaaggattgcgagacacggggcgatcacgtgttctgtgatacagccgccggaatcaacgtggccgagcagagcagagagtgcaacatcaacatcagcaccacaaactacccctgcaaggtgtccaccggcagacaccctatcagcatggtggctctgtctccactgggagccctggtggcttgttataagggcgtgtcctgtagcatcggcagcaatcaagtgggcatcatcaagcagctgcccaagggctgctcctacatcaccaatcaggacgccgacaccgtgaccatcgacaataccgtgtatcagctgagcaaggtggaaggcgaacagcacgtgatcaagggcagacctgtgtccaacagcttcgaccccatcagattccccgaggaccagttcaatgtggccctggaccaggtgttcgagagcatcgagaatagccaggctctggtggaccagtccaacaagatcctgaactccgccgagaagggcaacaccggcttcatcatcgtgatcatcctgattgccgtgctgggcctgaccatgatcagcgtgtccatcatcattatcatcaagaaaacgcggaagcccgccggcgcccctccagaacttaatggcgtgaccaacggcggcttcattccccactct

SEQ ID NO: 523 - protein, 5 aa, synthetic construct
SYGIS

SEQ ID NO: 524 - protein, 7 aa, synthetic construct
GYTFTSY

SEQ ID NO: 525 - protein, 17 aa, synthetic construct
WISGYNGNTNYAQTFQG

SEQ ID NO: 526 - protein, 6 aa, synthetic construct
SGYNGN

SEQ ID NO: 527 - protein, 14 aa, synthetic construct
DRYYASGSYNGMDV

SEQ ID NO: 528 - protein, 14 aa, synthetic construct
DRYYASGSYNGMDV

SEQ ID NO: 529 - protein, 13 aa, synthetic construct
SGSSSNVGSSSVY

SEQ ID NO: 530 - protein, 7 aa, synthetic construct
RNNQRPS

SEQ ID NO: 531 - protein, 11 aa, synthetic construct
AAWDDSLSGWV

SEQ ID NO: 532 - protein, 453 aa, synthetic construct
QVQLQQSGAEVKKPGASVKVSCKASGYTFTSYGISWVRQAPGQGLEWMGWISGYNGNTNYAQTFQGTFTMTTDTSTSTAYMELRRLRSGDTAVYYCARDRYYASGSYNGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 533 - protein, 216 aa, synthetic construct
QSALTQPPSASGAPGQRVTVSCSGSSSNVGSSSVYWYQQLPGTAPKLLIYRNNQRPSGVPDRFSGSKSGTSASLAISGLRSEDEADYYCAAWDDSLSGWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 534 - protein, 5 aa, synthetic construct
NYVIS

SEQ ID NO: 535 - protein, 7 aa, synthetic construct
GGTFSNY

SEQ ID NO: 536 - protein, 17 aa, synthetic construct
GIIPVFATAKYAQKFQG

SEQ ID NO: 537 - protein, 6 aa, synthetic construct
IPVFAT

SEQ ID NO: 538 - protein, 13 aa, synthetic construct
VDSGGTWWDAFAI

SEQ ID NO: 539 - protein, 13 aa, synthetic construct
VDSGGTWWDAFAI

SEQ ID NO: 540 - protein, 14 aa, synthetic construct
TGTSSDVGGYNYVS

SEQ ID NO: 541 - protein, 7 aa, synthetic construct
DVTKRPS

SEQ ID NO: 542 - protein, 10 aa, synthetic construct
CSYAGSYTLV

SEQ ID NO: 543 - protein, 452 aa, synthetic construct
QVQLVQSGAEVKKPGSSVKVSCEASGGTFSNYVISWVRQAPGQGLEWMGGIIPVFATAKYAQKFQGRVTIIADQSTNTAYLELSSLRSDDTAVYFCASVDSGGTWWDAFAIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 544 - protein, 216 aa, synthetic construct
QAVVTQPRSVSGSPGQAVTISCTGTSSDVGGYNYVSWYQHHPGKAPKLIIYDVTKRPSGVPDRFSGSKSGNTASLTISGLQTEYEADYYCCSYAGSYTLVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 545 - DNA, 369 bp, synthetic construct
  source 1..369: mol_type = other DNA
caggtgcagctacagcagtctggagctgaggtgaagaagcctggggcctctgtgaaggtctcatgcaaggcttctggttacacctttaccagctatggtatcagctgggtgcgccaggcccctggacaagggcttgagtggatggggtggatcagcggttacaatggaaacacaaactatgcacagacgttccagggcactttcaccatgaccacagacacatccacgagcacagcctacatggagctgaggaggctgcgatctggcgacacggccgtatactactgtgcgagagatcgctactatgcttcggggagttacaacggtatggacgtctggggccaagggaccacggtcactgtctcctca

SEQ ID NO: 546 - DNA, 330 bp, synthetic construct
  source 1..330: mol_type = other DNA
cagtctgccctgactcagccaccctcagcgtctggggcccccgggcagagggtcaccgtctcttgttctggaagcagctccaacgtcggaagtagttctgtatactggtaccagcagctgccaggaacggcccccaaactcctcatctataggaataatcagcggccctcaggggtccctgaccgattctctggctccaagtctggcacctcagcctccctggccatcagtgggctccggtccgaggatgaggctgattattactgtgcagcatgggatgacagcctgagtggttgggtcttcggcggagggaccaagctgaccgtccta

SEQ ID NO: 547 - DNA, 1359 bp, synthetic construct
  source 1..1359: mol_type = other DNA
caggtgcagctacagcagtctggagctgaggtgaagaagcctggggcctctgtgaaggtctcatgcaaggcttctggttacacctttaccagctatggtatcagctgggtgcgccaggcccctggacaagggcttgagtggatggggtggatcagcggttacaatggaaacacaaactatgcacagacgttccagggcactttcaccatgaccacagacacatccacgagcacagcctacatggagctgaggaggctgcgatctggcgacacggccgtatactactgtgcgagagatcgctactatgcttcggggagttacaacggtatggacgtctggggccaagggaccacggtcactgtctcctcagcgtcgaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggtaaa

SEQ ID NO: 548 - DNA, 647 bp, synthetic construct
  source 1..647: mol_type = other DNA
cagtctgccctgactcagccaccctcagcgtctggggcccccgggcagagggtcaccgtctcttgttctggaagcagctccaacgtcggaagtagttctgtatactggtaccagcagctgccaggaacggcccccaaactcctcatctataggaataatcagcggccctcaggggtccctgaccgattctctggctccaagtctggcacctcagcctccctggccatcagtgggctccggtccgaggatgaggctgattattactgtgcagcatgggatgacagcctgagtggttgggtcttcggcggagggaccaagctgaccgtcctaggtcacccaaggctgccccctcggtcactctgttcccgccctcctctgaggagcttcaagccaacaaggccacactggtgtgtctcataagtgacttctacccgggagccgtgacagtggcctggaaggcagatagcagccccgtcaaggcgggagtggagaccaccacaccctccaaacaaagcaacaacaagtacgcggccagcagctatctgagcctgacgcctgagcagtggaagtcccacagaagctacagctgccaggtcacgcatgaagggagcaccgtggagaagacagtggcccctacagaatgttca

SEQ ID NO: 549 - DNA, 366 bp, synthetic construct
  source 1..366: mol_type = other DNA
caggtccagcttgtacagtctggggctgaggtgaagaagcctgggtcctcggtgaaggtctcctgcgaggcttctggaggcaccttcagcaattacgttatcagttgggtgcgacaggcccctggacaagggcttgagtggatgggagggatcatcccagtctttgctacagcaaaatatgcacagaagttccagggcagagtcacgattatcgcggaccaatccacgaatacagcctacttggagctgagcagcctgagatctgacgacactgccgtttatttctgtgcgagcgtggatagtggtggtacttggtgggatgcttttgctatctggggccaagggacaatggtcaccgtctcctca

SEQ ID NO: 550 - DNA, 330 bp, synthetic construct
  source 1..330: mol_type = other DNA
caggctgtggtgacccagcctcgctcagtgtccgggtctcctggacaggcagtcaccatctcctgcactggaaccagcagtgatgttggtggttataactatgtctcctggtaccagcaccacccaggcaaagcccccaaactcatcatttatgatgtcactaagcggccctcaggggtccctgatcggttctctggctccaagtctggcaacacggcctccctgaccatctctgggctccagactgagtatgaggctgattattactgctgctcatatgcaggcagctacactttggttttcggcggagggaccaagctgaccgtccta

SEQ ID NO: 551 - DNA, 1356 bp, synthetic construct
  source 1..1356: mol_type = other DNA
caggtccagcttgtacagtctggggctgaggtgaagaagcctgggtcctcggtgaaggtctcctgcgaggcttctggaggcaccttcagcaattacgttatcagttgggtgcgacaggcccctggacaagggcttgagtggatgggagggatcatcccagtctttgctacagcaaaatatgcacagaagttccagggcagagtcacgattatcgcggaccaatccacgaatacagcctacttggagctgagcagcctgagatctgacgacactgccgtttatttctgtgcgagcgtggatagtggtggtacttggtgggatgcttttgctatctggggccaagggacaatggtcaccgtctcctcagcgtcgaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggtaaa

SEQ ID NO: 552 - DNA, 652 bp, synthetic construct
  source 1..652: mol_type = other DNA
caggctgtggtgacccagcctcgctcagtgtccgggtctcctggacaggcagtcaccatctcctgcactggaaccagcagtgatgttggtggttataactatgtctcctggtaccagcaccacccaggcaaagcccccaaactcatcatttatgatgtcactaagcggccctcaggggtccctgatcggttctctggctccaagtctggcaacacggcctccctgaccatctctgggctccagactgagtatgaggctgattattactgctgctcatatgcaggcagctacactttggttttcggcggagggaccaagctgaccgtcctaggtcagcccaaggctgccccctcggtcactctgttcccgccctcctctgaggagcttcaagccaacaaggccacactggtgtgtctcataagtgacttctacccgggagccgtgacagtggcctggaaggcagatagcagccccgtcaaggcgggagtggagaccaccacaccctccaaacaaagcaacaacaagtacgcggccagcagctatctgagcctgacgcctgagcagtggaagtcccacagaagctacagctgccaggtcacgcatgaagggagcaccgtggagaagacagtggcccctacagaatgttcatgag

SEQ ID NO: 553 - protein, 120 aa, synthetic construct
VQLQESGPGLVKPSETLSLTCTVSGGSVSSYYWSWIRQPPGKGLEWIGNIYYSGTTKYNPSLKSRVTISVDVSKNQFSLNLISVTAADTAVYFCARQVKSGWFVQPFDYWGQGALVTVSS

SEQ ID NO: 554 - protein, 105 aa, synthetic construct
QMTQSPPSLSAYVGDRVTITCRASQAIANYLAWFQQKPGKAPKSLIYAASTLQSGVPSRFSGSGSGTDFTLTISSLQPEDFATYYCHQYNTYPITFGQGTRLEIK

(19) United States
(12) Patent Application Publication
(10) Pub. No.: US 2024/0252614 A1
(43) Pub. Date: Aug. 1, 2024

(54) VACCINES AGAINST RESPIRATORY DISEASES

(71) Applicant: Pfizer Inc., New York, NY (US)

(72) Inventors: Damon Andrew Hollands Berman, Brewster, NY (US); Ye Che, Niantic, CT (US); Alexey Vyacheslavovitch Gribenko, New City, NY (US); Bridget Yih Jiin Huang, New York, NY (US); Weiqiang Li, Brooklyn, NY (US); Yan Li, Bronx, NY (US); Kena Anne Swanson, Pearl River, NY (US); Helen Chen Wang, Fort Lee, NJ (US); Sabine Susanne Wellnitz, Nanuet, NY (US); Kam Ho Wong, Westbury, NY (US); Qi Yang, Orangeburg, NY (US); Aiping Zhu, Fort Lee, NJ (US)

(21) Appl. No.: 18/413,734

(22) Filed: Jan. 16, 2024

Related U.S. Application Data
(60) Provisional application No. 63/611,808, filed on Dec. 19, 2023.
(60) Provisional application No. 63/610,006, filed on Dec. 14, 2023.
(60) Provisional application No. 63/586,506, filed on Sep. 29, 2023.
(60) Provisional application No. 63/480,504, filed on Jan. 18, 2023.

(51) Int. Cl. A61K 39/155; A61K 9/51; A61K 39/00; A61K 39/12; C07K 14/005; C07K 16/10

(52) U.S. Cl. CPC A61K 39/155; A61K 9/5123; A61K 39/12; C07K 14/005; C07K 16/10; A61K 2039/70

(57) ABSTRACT

The present disclosure relates to hMPV F, PIV3 F and PIV1 F protein mutants, nucleic acids or vectors encoding a hMPV F, PIV3 F and PIV1 F protein mutant, compositions comprising a hMPV F, PIV3 F and PIV1 F protein mutant or nucleic acid, and uses of the hMPV F, PIV3 F and PIV1 F protein mutants, nucleic acids or vectors, and compositions.

### RELATED APPLICATIONS

This application claims priority to U.S. Provisional Application No. 63/611,808 filed Dec. 19, 2023, U.S. Provisional Application No. 63/610,006 filed Dec. 14, 2023, U.S. Provisional Application No. 63/586,506 filed Sep. 29, 2023 and U.S. Provisional Application No. 63/480,504 filed Jan. 18, 2023. The entire content of each of the foregoing applications is herein incorporated by reference in its entirety.

### FIELD OF THE INVENTION

The present invention relates to vaccines in general and vaccines against respiratory viruses such as hMPV A, hMPV B, PIV1 and PIV3.

### REFERENCE TO SEQUENCE LISTING

This application is being filed electronically via EFS-Web and includes an electronically submitted sequence listing in .xml format. The .xml file contains a sequence listing entitled “PC072946A Sequence Listing.xml” created on Jan. 9, 2024 and having a size of 657 KB. The sequence listing contained in this .xml file is part of the specification and is incorporated herein by reference in its entirety.

### BACKGROUND OF THE INVENTION

Human paramyxoviruses and pneumoviruses are widespread pathogens, cause considerable disease burden, and include measles virus (MeV), mumps virus (MuV), respiratory syncytial virus (RSV), metapneumovirus (MPV), and parainfluenza virus types 1-4 (PIV1-4).

Human metapneumovirus (hMPV) is a respiratory virus that infects the lungs and breathing passages. HMPV is a clinically important respiratory viruses that result in substantial disease burden in children and account for significant pediatric hospitalization.

There is near ubiquitous infection by the age of five and re-infections continue to be a burden throughout life (van den Hoogen et al., 2001). However, infants (6-12 months), the elderly, and immunocompromised populations are at an increased risk of hospitalization with more severe disease such as pneumonia and bronchiolitis (Deffrasnes et al., 2007). Despite the disease burden that hMPV presents, there are no vaccines or therapeutics that have been approved for prevention or treatment.

hMPV is a member of the Pneumoviridae family, and its genome comprises three transmembrane surface glycoproteins: the attachment protein G, fusion protein F, and the small hydrophobic SH protein. There are two subtypes of hMPV, A and B. They differ primarily in the G glycoprotein, while the sequence of the F glycoprotein is more conserved between the two subtypes.

The mature F glycoprotein has three general domains: ectodomain (ED), transmembrane domain™, and a cytoplasmic tail©.

The F glycoprotein of hMPV is initially translated from the mRNA as a single 539-amino acid polypeptide precursor (referred to as “F0” or “F0 precursor”), which contains a signal peptide sequence (amino acids 1-18) at the N-terminus. Upon translation the signal peptide is removed by a signal peptidase in the endoplasmic reticulum.

The remaining portion of the F0 precursor (i.e., residues 18-539) may be further cleaved at position 102/103 by cellular proteases to generate two linked fragments designated F1 (C-terminal portion; amino acids 103-539) and F2 (N-terminal portion; amino acids 19-102). F1 contains a hydrophobic fusion peptide at its N-terminus and two heptad-repeat regions (HRA and HRB). HRA is near the fusion peptide, and HRB is near the TM domain. The F1 and F2 fragments are linked together through two disulfide bonds. Either the uncleaved F0 protein without the signal peptide sequence or a F1-F2 heterodimer can form a hMPV F protomer. Three such protomers assemble to form the final hMPV F protein complex, which is a homotrimer of the three protomers.

The F proteins of subtypes A and B are well conserved and an example sequence of the F0 precursor polypeptide for the A subtype is provided in SEQ ID NO: 1 (A2b strain (TN/95/3-54) GenBank GI: ACJ53569.1)), and for the B subtype is provided in SEQ ID NO: 4 (consensus sequence). SEQ ID NO:1 and SEQ ID NO:4 are both 539 amino acid sequences. The signal peptide sequence for SEQ ID NO:1 and SEQ ID NO:4 consists of amino acids 1-18.

One of the primary antigens explored for hMPV subunit vaccines is the F protein. The hMPV F protein trimer mediates fusion between the virion membrane and the host cellular membrane and also promotes the formation of syncytia. In the virion prior to fusion with the membrane of the host cell, the largest population of F molecules forms a lollipop-shaped structure, with the TM domain anchored in the viral envelope. This conformation is referred to as the prefusion conformation. Prefusion hMPV A F is recognized for example by monoclonal antibodies (mAbs) MPE8, without discrimination between oligomeric states. During hMPV entry into cells, the F protein rearranges from the prefusion state (which may be referred to herein as “pre-F”), through an intermediate extended structure, to a post-fusion state (“post-F”). During this rearrangement, the C-terminal coiled-coil of the prefusion molecule dissociates into its three constituent strands, which then wrap around the globular head and join three additional helices to form the post-fusion six helix bundle. If a prefusion hMPV F trimer is subjected to increasingly harsh chemical or physical conditions, such as elevated temperature, it undergoes structural changes. Initially, there is loss of trimeric structure (at least locally within the molecule), and then rearrangement to the post-fusion form, and then denaturation of the domains.

To prevent viral entry, F-specific neutralizing antibodies presumably must bind the prefusion conformation of F on the virion, or potentially the extended intermediate, before the viral envelope fuses with a cellular membrane. Thus, the prefusion form of the F protein is considered the preferred conformation as the desired vaccine antigen (Stewart Jones et al, PNAS 2021 Vol. 118 No. 39 and Hsieh et al, Nature Communications volume 13, Article number: 1299 (2022). However, the exact role of hMPV F prefusion form in eliciting immunogenicity is less established in comparison with RSV F. Upon extraction from a membrane with surfactants or expression as an ectodomain, physical or chemical stress, or storage, the F glycoprotein readily converts to the post-fusion form (Mas et al, 2016 PLoS Pathog 12(9): e1005859).

PIV1 and PIV3 (genus Respirovirus) are also important pediatric pathogens within the paramyxoviridae family, with lower incidence or disease severity caused by the paramyxovirus family members PIV2 and PIV4. While effective responses to measles and mumps can be induced by live attenuated viral vaccines, licensed vaccines for PIV1 and PIV3 have not been obtained using the same approach. Entry by these viruses also utilizes the viral fusion (F) glycoprotein, as disclosed above for hMPV.

The preparation of hMPV, PIV1 or PIV3 prefusion F as a vaccine antigen has remained a challenge. Since the neutralizing and protective antibodies function by interfering with virus entry, it is postulated that an F antigen that elicits only post-fusion specific antibodies is not expected to be as effective as an F antigen that elicits prefusion specific antibodies. Therefore, it is considered more desirable to utilize an F vaccine that contains a F protein immunogen in the prefusion form. Efforts to date have not yielded an hMPV, PIV1 or PIV3 vaccine that has been demonstrated in the clinic to elicit sufficient levels of protection to support licensure of an hMPV, PIV1 or PIV3 vaccine. Therefore, there is a need for immunogens derived from a hMPV, PIV1 and PIV3 F protein that have improved properties, such as increased expression for example when recombinantly expressed in mammalian cells, enhanced immunogenicity, or improved stability of the prefusion form, as compared with the corresponding native hMPV, PIV1 or PIV3 F protein, as well as compositions comprising such an immunogen, such as a vaccine.

There is also a need for respiratory vaccine comprising a combination of hMPV, PIV1 and/or PIV3 F protein antigen to provide protection against several virus causing respiratory diseases in a single vaccine.

### SUMMARY OF THE INVENTION

In some aspects, the present invention provides mutants of wild-type hMPV F proteins, wherein the mutants display introduced mutations in the amino acid sequence relative to the amino acid sequence of the corresponding wild-type hMPV F protein and are immunogenic against the wild-type hMPV F protein in the prefusion conformation or against a virus comprising the wild-type hMPV F protein. The amino acid mutations in the mutants include amino acid substitutions, deletions, or additions relative to a wild-type hMPV F protein.

In some embodiments, the present disclosure provides mutants of a wild-type hMPV F protein, wherein the introduced amino acid mutations are mutation of a pair of amino acid residues in a wild-type hMPV F protein to a pair of cysteines (“engineered disulfide mutation”). The introduced pair of cysteine residues allows for formation of a disulfide bond between the cysteine residues that stabilize the protein's conformation or oligomeric state, such as the prefusion conformation. Examples of specific pairs of such mutations include: 366C and 454C, 411C and 434C, 137C and 159C, 140C and 149C, 141C and 159C, 141C and 161C, 146C and 160C, 148C and 158C, and 150C and 156C, such as G366C and D454C, T411C and Q434C, 1137C and A159C, A140C and S149C, L141C and A159C, L141C and A161C, E146C and T160C, V148C and L158C and T150C and R156C.

In still other embodiments, the hMPV F protein mutants comprise amino acid mutations that are one or more cavity filling mutations. Examples of amino acids that may be replaced with the goal of cavity filling include small aliphatic (e.g. Gly, Ala, and Val) or small polar amino acids (e.g. Ser and Thr) and amino acids that are buried in the prefusion conformation, but exposed to solvent in the post-fusion conformation. Examples of the replacement amino acids include aliphatic amino acids (Val, lie, Leu and Met), aromatic amino acids (His, Phe, Tyr and Trp) and polar amino acids (Thr) with greater size than the replaced amino acids. In some specific embodiments, the hMPV F protein mutant comprises a cavity filling mutation at one or more positions, preferably one, two or three positions selected from 49, 149, 159, 291, 365 and 473. In some specific embodiments, the hMPV F protein mutant comprises a cavity filling mutation selected from the group consisting of:
(1) substitution of the amino acid at position 49, 291 or 365 with I, V, L, M, F, Y, or H;
(2) substitution of the amino acid at position 149 with T, V, or I;
(3) substitution of the amino acid at position 159 with V, I, or L;
(4) substitution of the amino acid at position 473 with F or W.

In some particular embodiments, a hMPV F protein mutant comprises at least one cavity filling mutation selected from the group consisting of: T49I, S149T, A159V, S2911, T365I and L473F.

In some particular embodiments, a hMPV F protein mutant comprises one, two orthree cavity filling mutations selected from the group consisting of: T49I, S149T, A159V, S2911, T365I and L473F.

In still other embodiments, the present disclosure provides hMPV F protein mutants, wherein the mutants comprise proline substitution mutations, which prevent the structural refolding that occurs during transit from the prefusion to post-fusion conformation. In some specific embodiments, the hMPV F protein mutant comprises of a proline substitution mutation selected from the group consisting of 66P, 110P, 132P, 145P, 187P, 449P and 459P, such as L66P, L110P, S132P, N145P, L187P, V449P and A459P. In a preferred embodiment, the hMPV F protein mutant comprises the proline substitution mutations A459P.

In still other embodiments, the present disclosure provides hMPV F protein mutants, wherein the mutants comprise glycine replacement mutations, which remove a glycine residue in the middle of an α-helix to improve protein stability.
In some specific embodiments, the hMPV F protein mutant comprises a glycine replacement mutation selected from the group consisting of G106A, G121A and G239A.
In a preferred embodiment, the hMPV F protein mutant comprises the glycine replacement mutation G239A.

In still other embodiments, the present disclosure provides hMPV F protein mutants, which comprise a combination of two or more different types of mutations selected from engineered disulfide mutations, cavity filling mutations, proline substitution mutations and glycine replacement mutations. In some particular embodiments, the present invention provides a mutant of a wild-type hMPV F protein, which comprises a combination of mutations relative to the corresponding wild-type hMPV F protein, wherein the combination of mutations is selected from the group consisting of:
(1) combination of 140C and 149C;
(2) combination of 140C, 149C, 411C and 434C;
(3) combination of 140C, 149C, 411C, 434C and 459P;
(4) combination of 140C, 149C, 411C, 434C and 365I;
(5) combination of 140C, 149C, 411C, 434C and G239A;
(6) combination of 140C, 149C, 411C, 434C, 459P, G239A, 49I and 365I;
(7) combination of 411C, 434C, 141C and 161C;
(8) combination of 411C, 434C, 141C, 161C and 459P;
(9) combination of 411C, 434C, 141C, 161C and 49I;
(10) combination of 411C, 434C, 141C, 161C and 365I;
(11) combination of 411C, 434C, 141C, 161C and G239A;
(12) combination of 411C, 434C, 141C, 161C and 149T;
(13) combination of 411C, 434C, 141C, 1610C, 459P, G239A, 49I, 149T and 365I; and, (14) combination of 4110C, 4340, 1460, 1600, 459P, G239A, 49I, 149T and 365I.

In some particular embodiments, the present invention provides a mutant of a wild-type hMPV F protein, which comprises a combination of mutations relative to the corresponding wild-type hMPV F protein, wherein the combination of mutations is selected from the group consisting of:
(1) combination of A140C and S149C;
(2) combination of A140C, S149C, T411C and Q434C;
(3) combination of A140C, S149C, T411C, Q434C and A459P;
(4) combination of A140C, S149C, T411C, Q434C and T365I;
(5) combination of A140C, S149C, T411C, Q434C and G239A;
(6) combination of A140C, S149C, T411C, Q434C, A459P, G239A, T49I and T365I;
(7) combination of T411C, Q434C, L141C and A161C;
(8) combination of T411C, Q434C, L141C, A161C and A459P;
(9) combination of T411C, Q434C, L141C, A161C and T49I;
(10) combination of T411C, Q434C, L141C, A161C and T365I;
(11) combination of T411C, Q434C, L141C, A161C and G239A;
(12) combination of T411C, Q434C, L141C, A161C and S149T;
(13) combination of T411C, Q434C, L141C, A161C, A459P, G239A, T49I, S149T and T365I; and,
(14) combination of T411C, Q434C, E146C, T160C, A459P, G239A, T49I, S149T and T365I.

In some particular embodiments, the present invention provides a mutant of a wild-type hMPV A F protein, which comprises a combination of mutations relative to the corresponding wild-type hMPV A F protein, wherein the combination of mutations is selected from the group consisting of:
(1) combination of A140C and S149C;
(2) combination of A140C, S149C, T411C and Q434C;
(3) combination of A140C, S149C, T411C, Q434C and A459P;
(4) combination of A140C, S149C, T411C, Q434C and T365I;
(5) combination of A140C, S149C, T411C, Q434C and G239;
(6) combination of A140C, S149C, T411C, Q434C, A459P, G239A, T49I and T365I;
(7) combination of T411C, Q434C, L141C and A161C;
(8) combination of T411C, Q434C, L141C, A161C and A459P;
(9) combination of T411C, Q434C, L141C, A161C and T49I;
(10) combination of T411C, Q434C, L141C, A161C and T365I;
(11) combination of T411C, Q434C, L141C, A161C and G239A;
(12) combination of T411C, Q434C, L141C, A161C and S149T;
(13) combination of T411C, Q434C, L141C, A161C, A459P, G239A, T49I, S149T and T365I; and,
(14) combination of T411C, Q434C, E146C, T160C, A459P, G239A, T49I, S149T and T365I.

In some particular embodiments, the present invention provides a mutant of a wild-type hMPV F protein, which comprises a combination of mutations relative to the corresponding wild-type hMPV F protein, wherein the combination of mutations is selected from the group consisting of
(1) T150C, R156C and A459P;
(2) T150C, R156C and T49I;
(3) T150C, R156C, T49I and A459P;
(4) A140C, S149C, T411C, and Q434C;
(5) L141C, A161C, T411C, and Q434C;
(6) A140C, S149C, T411C, Q434C, and A459P;
(7) A140C, S149C, G239A, T411C and Q434C;
(8) L141C, A161C, T411C, Q434C and A459P;
(9) L141C, A161C, G239A, T411C and Q434C;
(10) T49I, T150C, R156C, G239A and A459P;
(11) A140C, S149C, G239A, T411C, Q434C and A459P;
(12) T49I, A140C, S149C, G239A, T411C, Q434C and A459P;
(13) T49I, A140C, S149C, G239A, T365I, T411C, Q434C and A459P;
(14) L141C, A161C, G239A, T411C, Q434C and A459P;
(15) T49I, L141C, A161C, G239A, T411C, Q434C and A459P;
(16) L141C, A161C, S149T, G239A, T411C, Q434C and A459P;
(17) T49I, L141C, A161C, S149T, G239A, T411C, Q434C and A459P;
(18) T49I, L141C, A161C, S149T, G239A, T365I, T411C, Q434C and A459P;
(19) T49I, S149T and A459P;
(20) A140C, S149C and A459P;
(21) T49I, A140C and S149C;
(22) T49I, A140C, S149C and A459P;
(23) T49I, L141C, A161C, T411C and Q434C;
(24) T49I, L141C, A161C, T411C, Q434C and A459P;
(25) L141C, A161C and S149T;
(26) L141C, A161C, S149T and A459P;
(27) T49I, L141C, A161C and S149T, and,
(28) T49I, L141C, A161C, S149T and A459P.

In some particular embodiments, the present invention provides a mutant of a wild-type hMPV F protein, which comprises a combination of mutations relative to the corresponding wild-type hMPV F protein, wherein the combination of mutations is selected from the group consisting of
(1) L66P;
(2) L187P;
(4) A140C, S149C and L187P;
(5) T49I;
(6)T365I; and,
(7) T49I and T365I.

In some particular embodiments, the present invention provides a mutant of a wild-type hMPV F protein, which comprises a combination of mutations relative to the corresponding wild-type hMPV F protein, wherein the combination of mutations is selected from the group consisting of
(1) L187P, Q100R and S101R; and,
(2) A140C, S149C, L187P, Q100R and S101R.

In some particular embodiments, the present invention provides a mutant of a wild-type hMPV B F protein, which comprises a combination of mutations relative to the corresponding wild-type hMPV B F protein, wherein the combination of mutations is selected from the group consisting of
(1) T150C, R156C and A459P;
(2) T150C, R156C and T49I;
(3) T150C, R156C, T49I and A459P;
(4) A140C, S149C, T411C, and Q434C;
(5) L141C, A161C, T411C, and Q434C;
(6) A140C, S149C, T411C, Q434C, and A459P;
(7) A140C, S149C, G239A, T411C and Q434C;
(8) L141C, A161C, T411C, Q434C and A459P;
(9) L141C, A161C, G239A, T411C and Q434C;
(10) T49I, T150C, R156C, G239A and A459P;
(11) A140C, S149C, G239A, T411C, Q434C and A459P;
(12) T49I, A140C, S149C, G239A, T411C, Q434C and A459P;
(13) T49I, A140C, S149C, G239A, T365I, T411C, Q434C and A459P;
(14) L141C, A161C, G239A, T411C, Q434C and A459P;
(15) T49I, L141C, A161C, G239A, T411C, Q434C and A459P;
(16) L141C, A161C, S149T, G239A, T411C, Q434C and A459P;
(17) T49I, L141C, A161C, S149T, G239A, T411C, Q434C and A459P;
(18) T49I, L141C, A161C, S149T, G239A, T365I, T411C, Q434C and A459P;
(19) T49I, S149T and A459P;
(20) A140C, S149C and A459P;
(21) T49I, A140C and S149C;
(22) T49I, A140C, S149C and A459P;
(23) T49I, L141C, A161C, T411C and Q434C;
(24) T49I, L141C, A161C, T411C, Q434C and A459P;
(25) L141C, A161C and S149T;
(26) L141C, A161C, S149T and A459P;
(27) T49I, L141C, A161C and S149T, and,
(28) T49I, L141C, A161C, S149T and A459P.

In some particular embodiments, the present invention provides a mutant of a wild-type hMPV B F protein, which comprises a combination of mutations relative to the corresponding wild-type hMPV B F protein, wherein the combination of mutations is selected from the group consisting of
(1) L66P;
(2) L187P;
(4) A140C, S149C and L187P;
(5) T49I;
(6)T365I; and,
(7) T49I and T365I.

In some particular embodiments, the present invention provides a mutant of a wild-type hMPV B F protein, which comprises a combination of mutations relative to the corresponding wild-type hMPV B F protein, wherein the combination of mutations is selected from the group consisting of
(1) L187P, Q100R and S101R; and,
(2) A140C, S149C, L187P, Q100R and S101R.

In some aspects, the present invention provides mutants of wild-type PIV1 F proteins, wherein the mutants display introduced mutations in the amino acid sequence relative to the amino acid sequence of the corresponding wild-type PIV1 F protein and are immunogenic against the wild-type PIV1 F protein in the prefusion conformation or against a virus comprising the wild-type PIV1 F protein. The amino acid mutations in the mutants include amino acid substitutions, deletions, or additions relative to a wild-type PIV1 F protein.

In some embodiments, the present disclosure provides mutants of a wild-type PIV1 F protein, wherein the introduced amino acid mutations comprises at least one engineered disulfide mutation. Examples of specific pairs of such mutations include: Q92C-G134C.

In still other embodiments, the PIV1 F protein mutants comprise amino acid mutations that are one or more cavity filling mutations. In some specific embodiments, the PIV1 F protein mutant comprises a cavity filling mutation at one or more positions, preferably one, two or three position selected from 198, 92, 466, 473 or 480. In some particular embodiments, a PIV1 F protein mutant comprises at least one cavity filling mutation selected from the group consisting of T198A, Q92A, Q92L, A466L, A466V, A4661, S473V, S473L, S4731, S473A, A480L and A480V.

In still other embodiments, the present disclosure provides PIV1 F protein mutants, wherein the mutants comprise proline substitution mutations, which prevent the structural refolding that occurs during transit from the prefusion to post-fusion conformation.

In some specific embodiments, the PIV1 F protein mutant comprises of the proline substitution mutation A128P.

In still other embodiments, the present disclosure provides PIV1 F protein mutants, wherein the mutants comprise glycine replacement mutations, which remove a glycine residue in the middle of an α-helix to improve protein stability. In some specific embodiments, the PIV1 F protein mutant comprises a glycine replacement mutation selected from the group consisting of G134A or G134L.

In still other embodiments, the present disclosure provides PIV1 F protein mutants, wherein the mutants comprise a cleavage site mutation which prevents cleavage of the PIV1 F protein. In such case, the F1 and F2 polypeptides form a single polypeptide instead of two separate polypeptides linked by disulfide bonds. In some specific embodiments, the PIV1 F protein mutant comprises a the cleavage site mutations F113G and F114S.

In still other embodiments, the present disclosure provides PIV1 F protein mutants, which comprise a combination of two or more different types of mutations selected from engineered disulfide mutations, cavity filling mutations, proline substitution mutations, glycine replacement mutations and cleavage site mutations. In some particular embodiments, the present invention provides a mutant of a wild-type PIV1 F protein, which comprises a combination of mutations relative to the corresponding wild-type PIV1 F protein, wherein the combination of mutations is selected from the group consisting of:
(1) Q92C and G134C;
(2) A466L;
(3) A466V;
(4) S473V;
(5) S473L;
(6) A480L;
(7) A466L and S473A;
(8) A466L and S473L;
(9) T198A;
(10) G134A;
(11) A128P;
(12) F113G, F114S, Q92C, G134C, A466L, S473L and A480L;
(13) Q92C, G134C, A466L, S473L and A480L;
(14) Q92C, G134C, A466L and S473L;
(15) F113G, F114S, Q92C, G134C, A466V, S473V and A480V;
(16) Q92C, G134C, A466V, S473V and A480V;
(17) Q92C, G134C, A466V and S473V;
(18) F113G, F114S, A466L, S473L, A480L and G134A;
(19) A466L, S473L, A480L and G134A;
(20) A466L, S473L and G134A;
(21) F113G, F114S, A466L, S473L, A480L, Q92A and G134A;
(22) F113G, F114S, A466L, S473L and G134A;
(23) A466L, S473L, A480L, Q92A, G134A;
(24) A466L, S473L, Q92A, G134A;
(25) F113G, F114S, Q92L, G134A;
(26) A466L, S473L, A480L, Q92L and G134A;
(27) A466L, S473L, Q92L and G134A;
(28) F113G, F114S, A466L, S473L, A480L, Q92A and G134L;
(29) A466L, S473L, A480L, Q92A and G134L;
(30) F113G, F114S, Q92C, G134C, A4661, S4731 and A480L;
(31) F113G, F114S, Q92C, G134C, A4661 and, S4731; and,
(32) A4661, S4731, A480L, Q92L and G134A.

In some aspects, the present invention provides mutants of wild-type PIV3 F proteins, wherein the mutants display introduced mutations in the amino acid sequence relative to the amino acid sequence of the corresponding wild-type PIV3 F protein and are immunogenic against the wild-type PIV3 F protein in the prefusion conformation or against a virus comprising the wild-type PIV3 F protein. The amino acid mutations in the mutants include amino acid substitutions, deletions, or additions relative to a wild-type PIV3 F protein.

In some embodiments, the present disclosure provides mutants of a wild-type PIV3 F protein, wherein the introduced amino acid mutations comprises at least one engineered disulfide mutation. Examples of specific pairs of such mutations include: V175C-A202C, S160C-V170C, E209C-L234C, E209C, S233C, G85C-E209C and Q162C-L168C.

In still other embodiments, the PIV3 F protein mutants comprise amino acid mutations that are one or more cavity filling mutations. In some specific embodiments, the PIV3 F protein mutant comprises a cavity filling mutation at one or more positions, preferably one, two or three position selected from 277, 470, 477, 463 and 474. In some particular embodiments, a PIV3 F protein mutant comprises at least one cavity filling mutation selected from the group consisting of T277V, S470A, S470L, S477A, A463L, 1474F and 1474Y.

In still other embodiments, the present disclosure provides PIV3 F protein mutants, wherein the mutants comprise proline substitution mutations, which prevent the structural refolding that occurs during transit from the prefusion to post-fusion conformation. In some specific embodiments, the PIV3 F protein mutant comprises of the proline substitution mutation S164P and/or G219P.

In still other embodiments, the present disclosure provides PIV3 F protein mutants, wherein the mutants comprise glycine replacement mutations, which remove a glycine residue in the middle of an α-helix to improve protein stability. In some specific embodiments, the PIV3 F protein mutant comprises a glycine replacement mutation selected from the group consisting of G196A or G230A.

In still other embodiments, the present disclosure provides PIV3 F protein mutants, wherein the mutants comprise an electrostatic mutation which decreases ionic repulsion or increase ionic attraction between residues in a protein that are proximate to each other in the folded structure. In some specific embodiments, the PIV3 F protein mutant comprises the electrostatic mutation E182L and/or D455S.

In still other embodiments, the present disclosure provides PIV3 F protein mutants, which comprise a combination of two or more different types of mutations selected from engineered disulfide mutations, cavity filling mutations, proline substitution mutations, glycine replacement mutations and electrostatic mutations. In some particular embodiments, the present invention provides a mutant of a wild-type PIV3 F protein, which comprises a combination of mutations relative to the corresponding wild-type PIV3 F protein, wherein the combination of mutations is selected from the group consisting of:
(1) V175C and A202C;
(2) S160C and V170C;
(3) S164P;
(4) G196A;
(5) G219P;
(6) G230A;
(7) E182L;
(8) S470A;
(9) S477A;
(10) S470A and S477A;
(11) D455S;
(12) A463L;
(13) Q162C, L168C, S470A and S477A;
(14) S160C, V170C, S470A and S477A;
(15) G230A, S470A and S477A;
(16) A463L, S470A and S477A;
(17) E209C and L234C,
(18) A463L and S470L,
(19) S160C, V170C, E209C-L234C, A463L and S470L;
(20) S160C, V170C, E209C-L234C, A463L and 1474F;
(21) S160C, V170C, E209C-L234C, A463L, 1474F, F110G, F111S;
(22) S160C, V170C, A463L and S470L;
(23) Q162C, L168C, G230A, A463V and 1474Y;
(24) Q162C, L168C, G230A, S470A and S477A;
(25) Q162C, L168C, G230A and A463L;
(26) Q162C, L168C, G230A, A463L, S470A and S477A;
(27) S160C, V170C, G230A, A463V and 1474Y;
(28) S160C, V170C, G230A, S470A and S477A;
(29) S160C, V170C, G230A and A463L;
(30) S160C, V170C, G230A, A463L, S470A and S477A; and
(31) S160C, V170C and A463L;
(32) E209C and S233C;
(33) G85C and E209C;
(34) T277V;
(35) A463L and 1474F;
(36) A4631, S470I
(37) S160C, V170C, E209C, S233C, A463L and S470L;
(38) S160C, V170C, E209C, S233C, A463L and 1474F;
(39) S160C, V170C, G85C, E209C, A463L and S470L;
(40) S160C, V170C, G85C, E209C, A463L and 1474F;
(41) S160C, V170C, E209C, L234C, T277V, A463L and S470L;
(42) S160C, V170C, E209C, L234C, T277V, A463L and 1474F;
(43) S160C, V170C, E209C, S233C, T277V, A463L and S470L;
(44) S160C, V170C, E209C, S233C, T277V, A463L and 1474F;
(45) S160C, V170C, G85C, E209C, T277V, A463L and 1474F;
(46) S160C, V170C, E209C, L234C, D455S, A463L and S470L;
(47) S160C, V170C, E209C, S233C, D455S, A463L and S470L;
(48) S160C, V170C, G85C, E209C, D455S, A463L and S470L;
(49) S160C, V170C, E209C, L234C, T277V, D455S, A463L and S470L;
(50) S160C, V170C, E209C, S233C, T277V, D455S, A463L and S470L;
(51) S160C, V170C, G85C, E209C, T277V, D455S, A463L and S470L;
(52) S160C, V170C and S470L;
(53) R106G, T107S, E108A, R109S, S160C, V170C, E209C, L234C, A463L and S470L;
(54) R106G, T107S, E108A, R109S, S160C, V170C, E209C, S233C, A463L and S470L;
(55) R106G, T107S, E108A, R109S, S160C, V170C, G85C, E209C, A463L and S470L;
(56) F110G, F111S, S160C, V170C, E209C, L234C, A463L and S470L;
(57) F110G, F111S, S160C, V170C, E209C, S233C, A463L and S470L;
(58) F110G, F111S, S160C, V170C, A463L and S470L;
(59) F110G, F111S, S160C, V170C and S470L;
(60) S160C, V170C, A463L and S477L;
(61) S160C, V170C, E209C, L234C, A463L and S470L; and,
(62) S160C, V170C and S470L.

In some particular embodiments, the present invention provides a mutant of a wild-type PIV3 F protein, which comprises a combination of mutations relative to the corresponding wild-type PIV3 F protein, wherein the combination of mutations is selected from the group consisting of:
(1) G230A, S470A and S477A;
(2) S160C, V170C, G230A and A463L;
(3) S160C, V170C, S470A and S477A;
(4) S160C, V170C, G230A, S470A and S477A;
(5) S160C, V170C, G230A, A463L, S470A and S477A;
(6) S160C, V170C, E209C, L234C, A463L and S470L;
(7) S160C, V170C, E209C, L234C, A463L and 1474F;
(8) S160C, V170C, E209C, L234C, A463L, S470L, F110G, F111S; and,
(9) S160C, V170C, A463L and S470L, and,
(10) E209C and L234C.

In another aspect, the present invention provides nucleic acid molecules that encode a hMPV A, hMPV B, PIV1 or PIV3 F protein mutant described herein. In one embodiment, the present invention provides nucleic acid molecules that encode a hMPV A, hMPV B, PIV1 or PIV3 F protein mutant described herein. In a preferred embodiment, the nucleic acid is an RNA, more preferably an mRNA. In a preferred embodiment, the mRNA encodes a precursor F0 polypeptide that, when expressed in an appropriate cell, is processed into a full length hMPV A, hMPV B, PIV1 or PIV3 F protein mutant disclosed herein (e.g. comprising one or more mutations, a F1 polypeptide comprising the ectodomain, the transmembrane domain and the cytoplasmic domain and a F2 polypeptide). In a preferred embodiment, the nucleic acid is an mRNA comprising a chemically modified nucleotide. In a preferred embodiment, the nucleic acid is an mRNA comprising a chemically modified nucleotide, preferably 1-methylpseudouridine. Preferably, all the uridines of the RNA are replaced by 1-methylpseudouridine.

In another aspect, the invention provides immunogenic compositions that comprise (1) a hMPV A, hMPV B, PIV1 or PIV3 F protein mutant described in the disclosure, and/or (2) a nucleic acid, preferably mRNA or modRNA, or vector encoding such a hMPV A, hMPV B, PIV1 or PIV3 F protein mutant described in the disclosure.

In some embodiments, the Immunogenic composition comprises one, two, three or four mutants selected from the group consisting of:
(1) a hMPV A F protein mutant described in the disclosure or a nucleic acid, preferably mRNA, encoding such mutant;
(2) a hMPV B F protein mutant described in the disclosure or a nucleic acid, preferably mRNA, encoding such mutant;
(3) a PIV1 F protein mutant described in the disclosure or a nucleic acid, preferably mRNA, encoding such mutant; and,
a PIV3 F protein mutant described in the disclosure or a nucleic acid, preferably mRNA, encoding such mutant.

The present disclosure also relates to the use of a hMPV A, hMPV B, PIV1 or PIV3 F protein mutant, nucleic acids encoding a hMPV A, hMPV B, PIV1 or PIV3 F protein mutant, vectors for expressing a hMPV A, hMPV B, PIV1 or PIV3 F protein mutant, or compositions comprising a hMPV A, hMPV B, PIV1 or PIV3 F protein mutant or nucleic acids.

In several embodiments, the present disclosure provides a method of eliciting an immune response to hMPV A, hMPV B, PIV1 and/or PIV3 in a subject, comprising administering to the subject an effective amount of a hMPV A, hMPV B, PIV1 and/or PIV3 F protein mutant, a nucleic acid encoding a hMPV A, hMPV B, PIV1 and/or PIV3 F protein mutant, or a composition comprising a hMPV A, hMPV B, PIV1 and/or PIV3 F protein mutant or nucleic acid encoding such mutant.

### BRIEF DESCRIPTION OF THE DRAWINGS

FIG. 1A-1C provide a schematic representation of the hMPV precursor polypeptide F0 (FIG. 1A), PIV1 precursor polypeptide F0 (FIG. 1B) and PIV3 precursor polypeptide F0 (FIG. 1C).

FIG. 2A-2C provide a schematic representation of the hMPV-F modRNA (FIG. 2A), PIV3-F modRNA (FIG. 2B) and PIV1-F modRNA modRNA (FIG. 2C).

FIG. 3A provides 50% neutralizing titers in PD2 mouse sera raised against different recombinant hMPV F protein mutants with 3.0 μg F protein. Dotted line represents the limit of detection at 20.

FIG. 3B provides 50% neutralizing titers in PD2 mouse sera raised against different recombinant hMPV F protein mutants with 1.0 μg F protein. Dotted line represents the limit of detection at 20.

FIG. 3C provides 50% neutralizing titers in PD2 mouse sera raised against different recombinant hMPV F protein mutants with 1.0 μg F protein and LiNA-2 adjuvant. Dotted line represents the limit of detection at 20.

FIG. 4 provides 50% neutralizing titers in PD2 mouse sera raised against different hMPV F protein mutants with 0.5 μg LNP-formulated modRNA. Dotted line represents the limit of detection at 20.

FIG. 5A provides 50% neutralizing titers in PD2 mouse sera raised against different recombinant PIV3 F protein mutants with 0.25 μg F protein. Dotted line represents the limit of detection at 20.

FIG. 5B provides 50% neutralizing titers in PD2 mouse sera raised against different recombinant PIV3 F protein mutants with 1.0 μg F protein. Dotted line represents the limit of detection at 20.

FIG. 5C provides 50% neutralizing titers in PD2 mouse sera raised against different recombinant PIV3 F protein mutants with 1.0 μg F protein and LiNA-2 adjuvant. Dotted line represents the limit of detection at 20.

FIG. 6A provides 50% neutralizing titers in PD2 mouse sera raised against different PIV3 F protein designs with 0.05 μg LNP-formulated modRNA. Dotted line represents the limit of detection at 20.

FIG. 6B provides 50% neutralizing titers in PD2 mouse sera raised against different PIV3 F protein designs with 0.2 μg LNP-formulated modRNA. Dotted line represents the limit of detection at 20.

FIG. 7A provides 50% neutralizing titers in PD2 mouse sera raised against different recombinant PIV1 F protein mutants with 2.0 μg F protein. Dotted line represents the limit of detection at 20.

FIG. 7B provides 50% neutralizing titers in PD2 mouse sera raised against different recombinant PIV1 F protein mutants with 0.5 μg F protein and LiNA-2 adjuvant. Dotted line represents the limit of detection at 20.

FIG. 8 provides. 50% neutralizing titers in PD2 mouse sera raised against different PIV1 F protein designs with 0.2 μg LNP-formulated modRNA. Dotted line represents the limit of detection at 20.

FIG. 9A provides 50% neutralizing titers in PD2 mouse sera raised against different recombinant PIV3 F protein mutants with 1.0 μg F protein. Dotted line represents the limit of detection at 20.

FIG. 9B provides 50% neutralizing titers in PD2 mouse sera raised against different recombinant PIV3 F protein mutants with 0.5 μg F protein and LiNA-2 adjuvant. Dotted line represents the limit of detection at 20.

FIG. 10A provides 50% neutralizing titers in PD2 mouse sera raised against different PIV3 F protein designs with 0.05 μg LNP-formulated modRNA. Dotted line represents the limit of detection at 20.

FIG. 10B provides 50% neutralizing titers in PD2 mouse sera raised against different PIV3 F protein designs with 0.2 μg LNP-formulated modRNA. Dotted line represents the limit of detection at 20.

### DETAILED DESCRIPTION OF THE INVENTION

### A. Definitions

As used herein, the singular forms “a,” “an,” and “the,” refer to both the singular as well as plural, unless the context clearly indicates otherwise. For example, the term “an antigen” includes single or plural antigens and can be considered equivalent to the phrase “at least one antigen.”

The term “adjuvant” refers to a substance capable of enhancing, accelerating, or prolonging the body's immune response to the antigen in a vaccine (although it is not the target antigen of the vaccine itself). An adjuvant may be included in the vaccine composition, or may be administered separately from the vaccine.

The term “administration” refers to the introduction of a substance or composition into a subject by a chosen route. Administration can be local or systemic. For example, if the chosen route is intramuscular, the composition (such as a composition including a disclosed immunogen) is administered by introducing the composition into a muscle of the subject.

An “antibody” refers to an immunoglobulin molecule capable of specific binding to a target, such as a polypeptide, carbohydrate, polynucleotide, lipid, etc., through at least one antigen binding site, located in the variable region of the immunoglobulin molecule. As used herein, the term “antibody” can encompass any type of antibody (e.g. monospecific, bispecific), and includes portions of intact antibodies that retain the ability to bind to a given antigen (e.g. an “antigen-binding fragment”), and any other modified configuration of an immunoglobulin molecule that comprises an antigen binding site. An antibody includes an antibody of any class, such as IgG, IgA, or IgM (or sub-class thereof), and the antibody need not be of any particular class. Depending on the antibody amino acid sequence of the constant region of its heavy chains (HC), immunoglobulins can be assigned to different classes. There are five major classes of immunoglobulins: IgA, IgD, IgE, IgG, and IgM, and several of these may be further divided into subclasses (isotypes), e.g., IgG₁, IgG₂, IgG₃, IgG₄, IgA₁ and IgA₂. The heavy chain constant regions that correspond to the different classes of immunoglobulins are called alpha, delta, epsilon, gamma, and mu, respectively. The subunit structures and three-dimensional configurations of different classes of immunoglobulins are well known. Examples of antibody antigen-binding fragments and modified configurations include (i) a Fab fragment (a monovalent fragment consisting of the VL, VH, CL and CH1 domains); (ii) a F(ab′)2 fragment (a bivalent fragment comprising two Fab fragments linked by a disulfide bridge at the hinge region); and (iii) a Fv fragment consisting of the VL and VH domains of a single arm of an antibody. Furthermore, although the two domains of an Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv)); see e.g., Bird et al., Science 1988; 242:423-426 and Huston et al., Proc. Natl. Acad. Sci. 1988 USA 85:5879-5883. Other forms of single chain antibodies, such as diabodies are also encompassed.

In addition, further encompassed are antibodies that are missing a C-terminal lysine (K) amino acid residue on a heavy chain polypeptide (e.g. human IgG1 heavy chain comprises a terminal lysine). As is known in the art, the C-terminal lysine is sometimes clipped during antibody production, resulting in an antibody with a heavy chain lacking the C-terminal lysine. Alternatively, an antibody heavy chain may be produced using a nucleic acid that does not include a C-terminal lysine.

The term “antigen” refers to a molecule that can be recognized by an antibody. Examples of antigens include polypeptides, peptides, lipids, polysaccharides, and nucleic acids containing antigenic determinants, such as those recognized by an immune cell.

An “agonist” refers to a substance which promotes (e.g., induces, causes, enhances, or increases) the biological activity or effect of another molecule. The term agonist encompasses substances (such as an antibody) which bind to a molecule to promote the activity of that molecule.

An “antagonist” refers to a substance that prevents, blocks, inhibits, neutralizes, or reduces a biological activity or effect of another molecule, such as a receptor. The term antagonist encompasses substances (such as an antibody) which bind to a molecule to prevent or reduce the activity of that molecule.

The term “binding affinity” refers to the strength of the sum total of noncovalent interactions between a single binding site of a molecule (e.g., an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a molecule X for its partner Y can generally be represented by the dissociation constant (K_{D}). Affinity can be measured by common methods known in the art. Low-affinity antibodies generally bind antigen slowly and tend to dissociate readily, whereas high-affinity antibodies generally bind antigen faster and tend to remain bound longer. In particular, the term “binding affinity” is intended to refer to the dissociation rate of a particular antigen-antibody interaction. The K_{D} is the ratio of the rate of dissociation, also called the “off-rate (k_{off})” or “k_{d}” to the association rate, or “on-rate (kₒₙ)” or “kₐ”. Thus, K_{D} equals k_{off}/kₒₙ (or k_{d}/kₐ) and is expressed as a molar concentration (M). It follows that the smaller the K_{D}, the stronger the affinity of binding. Therefore, a K_{D} of 1 μM indicates weaker binding affinity compared to a K_{D} of 1 nM. K_{D} values for antibodies can be determined using methods well established in the art. One exemplary method for determining the K_{D} of an antibody is by using surface plasmon resonance (SPR), typically using a biosensor system such as BIACORE system. BIACORE kinetic analysis comprises analyzing the binding and dissociation of an antigen from chips with immobilized molecules (e.g., molecules comprising epitope binding domains), on their surface. Another method for determining the K_{D} of an antibody is by using Bio-Layer Interferometry, typically using OCTET® technology (Octet QK^{e}-system, ForteBio). Alternatively, or in addition, a KinExA (Kinetic Exclusion Assay) assay, available from Sapidyne Instruments (Boise, ID) can also be used.

A “bispecific antibody” refers to a molecule that has binding specificity for at least two different epitopes. In some embodiments, bispecific antibodies can bind simultaneously two different antigens. In other embodiments, the two different epitopes may reside on the same antigen.

A “chimeric antibody” refers to an antibody in which the variable region sequences are derived from one species and the constant region sequences are derived from another species, such as an antibody in which the variable region sequences are derived from a mouse antibody and the constant region sequences are derived from a human antibody.

The term “compete”, as used herein with regard to an antibody, means that a first antibody binds to an epitope in a manner sufficiently similar to the binding of a second antibody such that the result of binding of the second antibody with its cognate epitope is detectably decreased in the presence of the first antibody compared to the binding of the second antibody in the absence of the first antibody. The alternative, where the binding of the first antibody to its epitope is also detectably decreased in the presence of the second antibody, can, but need not be the case. That is, a first antibody can inhibit the binding of a second antibody to its epitope without that second antibody inhibiting the binding of the first antibody to its respective epitope. However, where each antibody detectably inhibits the binding of the other antibody with its cognate epitope or ligand, whether to the same, greater, or lesser extent, the antibodies are said to “cross-compete” with each other for binding of their respective epitope(s). Both competing and cross-competing antibodies are encompassed by the present invention.

Regardless of the mechanism by which such competition or cross-competition occurs (e.g., steric hindrance, conformational change, or binding to a common epitope, or portion thereof), the skilled artisan would appreciate, based upon the teachings provided herein, that such competing or cross-competing antibodies are encompassed and can be useful for the methods disclosed herein.

The term “conservative substitution” refers to the substitution of an amino acid with a chemically similar amino acid. Conservative amino acid substitutions providing functionally similar amino acids are well known in the art. The following six groups each contain amino acids that are conservative substitutions for one another:
1) alanine (A), serine (S), threonine (T);
2) aspartic acid (D), glutamic acid (E);
3) asparagine (N), glutamine (Q);
4) arginine (R), lysine (K);
5) isoleucine (I), leucine (L), methionine (M), valine (V); and
6) phenylalanine (F), tyrosine (Y), tryptophan (W).

A “constant region” of an antibody refers to the constant region of the antibody light chain or the constant region of the antibody heavy chain, either alone or in combination. An IgG heavy chain constant region contains three sequential immunoglobulin domains (CH1, CH2, and CH3), with a hinge region between the CH1 and CH2 domains. An IgG light chain constant region contains a single immunoglobulin domain (CL).

The term “degenerate variant” of a reference polynucleotide refers to a polynucleotide that differs in the nucleotide sequence from the reference polynucleotide but encodes the same polypeptide sequence as encoded by the reference polynucleotide. There are 20 natural amino acids, most of which are specified by more than one codon. For instance, the codons CGU, CGC, CGA, CGG, AGA, and AGG all encode the amino acid arginine. Thus, at every position where an arginine is specified within a protein encoding sequence, the codon can be altered to any of the corresponding codons described without altering the encoded protein. Because of the degeneracy of the genetic code, a large number of functionally identical nucleic acids encode any given polypeptide.

The term “effective amount” refers to an amount of agent that is sufficient to generate a desired response. For instance, this can be the amount necessary to inhibit viral replication or to measurably alter outward symptoms of the viral infection.

An “effector cell” refers to a leukocyte which express one or more FcRs and performs effector functions. In certain embodiments, effector cells express at least FcgRIII and perform ADCC effector function(s). Examples of leukocytes which mediate ADCC include peripheral blood mononuclear cells (PBMC), natural killer (NK) cells, monocytes, macrophages, cytotoxic T cells, and neutrophils. Effector cells may be isolated from a native source, e.g., from blood.

The term “epitope” (or “antigenic determinant” or “antigenic site”) refers to the region of an antigen to which an antibody, B cell receptor, or T cell receptor binds or responds. Epitopes can be formed from contiguous amino acids or noncontiguous amino acids juxtaposed by secondary, tertiary, or quaternary folding of a protein. Epitopes formed from contiguous amino acids are typically retained on exposure to denaturing solvents whereas epitopes formed by higher order folding are typically lost on treatment with denaturing solvents.

The term “F0 polypeptide” (F0) when used in connection with hMPV F protein, refers to the precursor polypeptide of the hMPV F protein, which is composed of a signal polypeptide sequence, a F1 polypeptide sequence and a F2 polypeptide sequence. With rare exceptions the F0 polypeptides of the known hMPV strains consist of 539 amino acids.

The term “F0 polypeptide” (F0) when used in connection with PIV1 F protein, refers to the precursor polypeptide of the PIV 1 F protein, which is composed of a signal polypeptide sequence, a F1 polypeptide sequence and a F2 polypeptide sequence. Examples of F0 polypeptides of known PIV1 strains are provided in Table 4 and consist of 555 amino acids.

The term “F0 polypeptide” (F0) when used in connection with PIV3 F protein, refers to the precursor polypeptide of the PIV3 F protein, which is composed of a signal polypeptide sequence, a F1 polypeptide sequence and a F2 polypeptide sequence. Examples of F0 polypeptides of known PIV1 strains are provided in Table 6 and consist of 539 amino acids.

The term “F1 polypeptide” (F1) when used in connection with hMPV F protein refers to a polypeptide chain of a mature hMPV F protein. Native F1 includes approximately residues 103-539 of the hMPV F0 precursor and is composed of from N- to C-terminus) an extracellular region (approximately residues 103-489), a transmembrane domain (approximately residues 490-514), and a cytoplasmic domain (also referred to as intracellular domain) (approximately residues 515-539). As used herein, the term encompasses both native F1 polypeptides and F1 polypeptides including modifications (e.g., amino acid substitutions, insertions, or deletion) from the native sequence, for example, modifications designed to stabilize a F mutant or to enhance the immunogenicity of a F mutant.

The term “F1 polypeptide” (F1) when used in connection with PIV1 F protein refers to a polypeptide chain of a mature PIV1 F protein. Native F1 includes approximately residues 113-555 of the PIV1 F0 precursor and is composed of from N- to C-terminus) an extracellular region (approximately residues 103-496), a transmembrane domain (approximately residues 497-517), and a cytoplasmic domain (also referred to as intracellular domain) (approximately residues 518-555). As used herein, the term encompasses both native F1 polypeptides and F1 polypeptides including modifications (e.g., amino acid substitutions, insertions, or deletion) from the native sequence, for example, modifications designed to stabilize a F mutant or to enhance the immunogenicity of a F mutant.

The term “F1 polypeptide” (F1) when used in connection with PIV3 protein refers to a polypeptide chain of a mature PIV3 F protein. Native F1 includes approximately residues 103-539 of the PIV3 F0 precursor and is composed of from N- to C-terminus) an extracellular region (approximately residues 103-493), a transmembrane domain (approximately residues 494-514), and a cytoplasmic domain (also referred to as intracellular domain) (approximately residues 515-539). As used herein, the term encompasses both native F1 polypeptides and F1 polypeptides including modifications (e.g., amino acid substitutions, insertions, or deletion) from the native sequence, for example, modifications designed to stabilize a F mutant or to enhance the immunogenicity of a F mutant.

The term “F2 polypeptide” (F2) when used in connection with hMPV F protein refers to the polypeptide chain of a mature hMPV F protein. Native F2 includes approximately residues 19-102 of the hMPV F0 precursor. As used herein, the term encompasses both native F2 polypeptides and F2 polypeptides including modifications (e.g., amino acid substitutions, insertions, or deletion) from the native sequence, for example, modifications designed to stabilize a F mutant or to enhance the immunogenicity of a F mutant. In native hMPV F protein, the F2 polypeptide is linked to the F1 polypeptide by two disulfide bonds to form a F2-F1 heterodimer.

The term “F2 polypeptide” (F2) when used in connection with PIV1 protein refers to the polypeptide chain of a mature PIV1 F protein. Native F2 includes approximately residues 22-112 of the PIV1 F0 precursor. As used herein, the term encompasses both native F2 polypeptides and F2 polypeptides including modifications (e.g., amino acid substitutions, insertions, or deletion) from the native sequence, for example, modifications designed to stabilize a F mutant or to enhance the immunogenicity of a F mutant. In native PIV2 F protein, the F2 polypeptide is linked to the F1 polypeptide by two disulfide bonds to form a F2-F1 heterodimer.

The term “F2 polypeptide” (F2) when used in connection with PIV3 F protein refers to the polypeptide chain of a mature PIV3 F protein. Native F2 includes approximately residues 19-109 of the PIV3 F0 precursor. As used herein, the term encompasses both native F2 polypeptides and F2 polypeptides including modifications (e.g., amino acid substitutions, insertions, or deletion) from the native sequence, for example, modifications designed to stabilize a F mutant or to enhance the immunogenicity of a F mutant. In native PIV3 F protein, the F2 polypeptide is linked to the F1 polypeptide by two disulfide bonds to form a F2-F1 heterodimer.

A “Fc domain” refers to the portion of an immunoglobulin (Ig) molecule that correlates to a crystallizable fragment obtained by papain digestion of an Ig molecule. As used herein, the term relates to the 2-chained constant region of an antibody, each chain excluding the first constant region immunoglobulin domain. Within an Fc domain, there are two “Fc chains” (e.g. a “first Fc chain” and a “second Fc chain”). “Fc chain” generally refers to the C-terminal portion of an antibody heavy chain. Thus, Fc chain refers to the last two constant region immunoglobulin domains (CH2 and CH3) of IgA, IgD, and IgG heavy chains, and the last three constant region immunoglobulin domains of IgE and IgM heavy chains, and optionally the flexible hinge N-terminal to these domains.

Although the boundaries of the Fc chain may vary, the human IgG heavy chain Fc chain is usually defined to comprise residues C226 or P230 to its carboxyl-terminus, wherein the numbering is according to the EU index of Edelman et al., Proc. Natl. Acad. Sci. USA 1969; 63(1):78-85 and as described in Kabat et al., 1991. Typically, the Fc chain comprises from about amino acid residue 236 to about 447 of the human IgG1 heavy chain constant region. “Fc chain” may refer to this polypeptide in isolation, or in the context of a larger molecule (e.g. in an antibody heavy chain or Fc fusion protein).

A “functional” Fc domain refers to an Fc domain that possesses at least one effector function of a native sequence Fc domain. Exemplary “effector functions” include Clq binding; complement dependent cytotoxicity (CDC); Fc receptor binding; antibody-dependent cell-mediated cytotoxicity (ADCC); phagocytosis; down-regulation of cell surface receptors (e.g., B cell receptor); and B cell activation, etc. Such effector functions generally require the Fc domain to be combined with a binding domain (e.g., an antibody variable region) and can be assessed using various assays known in the art for evaluating such antibody effector functions.

A “native sequence” Fc chain refers to a Fc chain that comprises an amino acid sequence identical to the amino acid sequence of an Fc chain found in nature. A “variant” Fc chain comprises an amino acid sequence which differs from that of a native sequence Fc chain by virtue of at least one amino acid modification

An “Fc receptor” (FcR) refers to a receptor that binds to the Fc region of an antibody. In some embodiments, an FcR is a native human FcR. In some embodiments, an FcR is one which binds an IgG antibody (a gamma receptor) and includes receptors of the FcgRI, FcgRII, and FcgRIII subclasses, including allelic variants and alternatively spliced forms of those receptors. FcgRII receptors include FcgRIIA (an “activating receptor”) and FcgRIIB (an “inhibiting receptor”), which have similar amino acid sequences that differ primarily in the cytoplasmic domains thereof. Activating receptor FcgRIIA contains an immunoreceptor tyrosine-based activation motif (ITAM) in its cytoplasmic domain. Inhibiting receptor FcgRIIB contains an immunoreceptor tyrosine-based inhibition motif (ITIM) in its cytoplasmic domain, (see, e.g., Daeron, Annu. Rev. Immunol. 1997; 15:203-234). FcRs are reviewed, for example, in Ravetch and Kinet, Annu. Rev. Immunol 1991; 9:457-92; Capel et al., Immunomethods 1994; 4:25-34; and de Haas et al., J. Lab. Clin. Med. 1995; 126:330-41. Other FcRs, including those to be identified in the future, are encompassed by the term “Fc receptor” herein. The term “Fc receptor” also includes the neonatal receptor, FcRn, which is responsible for the transfer of maternal IgGs to the fetus (Guyer et al., J. Immunol. 1976; 117:587 and Kim et al., J. Immunol. 1994; 24:249) and regulation of homeostasis of immunoglobulins. Methods of measuring binding to FcRn are known (see, e.g., Ghetie and Ward., Immunol. Today 1997; 18(12):592-598; Ghetie et al., Nature Biotechnology, 1997; 15(7):637-640; Hinton et al., J. Biol. Chem. 2004; 279(8):6213-6216; WO 2004/92219).

The term “foldon” or “foldon domain” refers to an amino acid sequence that is capable of forming trimers. One example of such foldon domains is the peptide sequence derived from bacteriophage T4 fibritin, which has the sequence of GYIPEAPRDGQAYVRKDGEWVLLSTFL (SEQ ID NO:7).

The term “mammal” refers to any animal species of the Mammalia class. Examples of mammals include: humans; non-human primates such as monkeys; laboratory animals such as rats, mice, guinea pigs; domestic animals such as cats, dogs, rabbits, cattle, sheep, goats, horses, and pigs; and captive wild animals such as lions, tigers, elephants, and the like.

The term “glycoprotein” refers to a protein that contains oligosaccharide chains (glycans) covalently attached to polypeptide side-chains. The carbohydrate is attached to the protein in a cotranslational or posttranslational modification known as glycosylation. The term “glycosylation site” refers to an amino acid sequence on the surface of a polypeptide, such as a protein, which accommodates the attachment of a glycan. An N-linked glycosylation site is triplet sequence of NX(S/T) in which N is asparagine, X is any residue except proline, and (S/T) is a serine or threonine residue. A glycan is a polysaccharide or oligosaccharide. Glycan may also be used to refer to the carbohydrate portion of a glycoconjugate, such as a glycoprotein, glycolipid, or a proteoglycan.

A “monoclonal antibody” (mAb) refers to an antibody that is derived from a single copy or clone, including e.g., any eukaryotic, prokaryotic, or phage clone. Monoclonal antibodies are highly specific, being directed against a single antigenic site. Furthermore, in contrast to polyclonal antibody preparations, which typically include different antibodies directed against different determinants (epitopes), each monoclonal antibody is directed against a single determinant on the antigen. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma method first described by Kohler and Milstein, 1975, Nature 256:495, or may be made by recombinant DNA methods such as described in U.S. Pat. No. 4,816,567. In another example, monoclonal antibodies may be isolated from phage libraries such as those generated using the techniques described in McCafferty et al., 1990, Nature 348:552-554.

A “monospecific antibody” refers to an antibody that comprises one or more antigen binding sites per molecule such that any and all binding sites of the antibody specifically recognize the identical epitope on the antigen. Thus, in cases where a monospecific antibody has more than one antigen binding site, the binding sites compete with each other for binding to one antigen molecule.

The term “hMPV-2 mAb” refers to an hMPV A F protein prefusion specific antibody which has a heavy chain variable domain comprising an amino acid sequence of SEQ ID NO:360 and a light chain variable domain comprising an amino acid sequence of SEQ ID NO:361.

The term “half maximal effective concentration (EC₅₀)” refers to the concentration of a therapeutic agent which causes a response halfway between the baseline and maximum after a specified exposure time. The therapeutic agent may cause inhibition or stimulation. The EC₅₀ value is commonly used, and is used herein, as a measure of potency.

The term “host cells” refers to cells in which a vector can be propagated and its DNA or RNA expressed. The cell may be prokaryotic or eukaryotic.

A “human antibody” refers to an antibody which possesses an amino acid sequence which corresponds to that of an antibody produced by a human or has been made using any technique for making fully human antibodies. For example, fully human antibodies may be obtained by using commercially available mice that have been engineered to express specific human immunoglobulin proteins, or by library (e.g. phage, yeast, or ribosome) display techniques for preparing fully human antibodies. This definition of a human antibody specifically excludes a humanized antibody comprising non-human antigen binding residues.

A “humanized” antibody refers to a non-human (e.g. murine) antibody that is a chimeric antibody that contains minimal sequence derived from non-human immunoglobulin. Preferably, humanized antibodies are human immunoglobulins (recipient antibody) in which residues from a CDR of the recipient are replaced by residues from a CDR of a non-human species (donor antibody) such as mouse, rat, or rabbit having the desired specificity, affinity, and capacity. The humanized antibody may comprise residues that are found neither in the recipient antibody nor in the imported CDR or framework sequences, but are included to further refine and optimize antibody performance. The term “identical” or percent “identity,” in the context of two or more nucleic acid or polypeptide sequences, refers to two or more sequences or subsequences that are the same or have a specified percentage of amino acid residues or nucleotides that are the same, when compared and aligned for maximum correspondence. Methods of alignment of sequences for comparison are well known in the art. Once aligned, the number of matches is determined by counting the number of positions where an identical nucleotide or amino acid residue is present in both sequences. The percent sequence identity is determined by dividing the number of matches either by the length of the sequence set forth in the identified sequence, or by an articulated length (such as 100 consecutive nucleotides or amino acid residues from a sequence set forth in an identified sequence), followed by multiplying the resulting value by 100. For example, a peptide sequence that has 1166 matches when aligned with a test sequence having 1554 amino acids is 75.0 percent identical to the test sequence (1166+1554*100=75.0).

Optimal alignment of sequences for comparison can be conducted, e.g., by the local homology algorithm of Smith and Waterman, Adv. Appl. Math. 2:482, 1981, by the homology alignment algorithm of Needleman and Wunsch, Mol. Biol. 48:443, 1970, by the search for similarity method of Pearson and Lipman, Proc. Nat'l. Acad. Sci. USA 85:2444, 1988, by computerized implementations of these algorithms (GAP, BESTFIT, FASTA, and TFASTA in the Wisconsin Genetics Software Package, Genetics Computer Group, 575 Science Dr., Madison, WI), or by manual alignment and visual inspection (see, e.g., Sambrook et al. (Molecular Cloning: A Laboratory Manual, 4th ed, Cold Spring Harbor, New York, 2012) and Ausubel et al. (In Current Protocols in Molecular Biology, John Wiley and Sons, New York, through supplement 104, 2013).

The term “immunogenic” refers to the ability of a substance to cause, elicit, stimulate, or induce an immune response against a particular antigen, in an animal, whether in the presence or absence of an adjuvant.

The term “immune response” refers to any detectable response of a cell or cells of the immune system of a host mammal to a stimulus (such as an immunogen), including, but not limited to, innate immune responses (e.g., activation of Toll receptor signaling cascade), cell-mediated immune responses (e.g., responses mediated by T cells, such as antigen-specific T cells, and non-specific cells of the immune system), and humoral immune responses (e.g., responses mediated by B cells, such as generation and secretion of antibodies into the plasma, lymph, and/or tissue fluids). Examples of immune responses include an alteration (e.g., increase) in Toll-like receptor activation, lymphokine (e.g., cytokine (e.g., Th1, Th2 or Th17 type cytokines) or chemokine) expression or secretion, macrophage activation, dendritic cell activation, T cell (e.g., CD4+ or CD8+ T cell) activation, NK cell activation, B cell activation (e.g., antibody generation and/or secretion), binding of an immunogen (e.g., antigen (e.g., immunogenic polypeptide)) to an MHC molecule, induction of a cytotoxic T lymphocyte (“CTL”) response, induction of a B cell response (e.g., antibody production), and, expansion (e.g., growth of a population of cells) of cells of the immune system (e.g., T cells and B cells), and increased processing and presentation of antigen by antigen presenting cells. The term “immune response” also encompasses any detectable response to a particular substance (such as an antigen or immunogen) by one or more components of the immune system of a vertebrate animal in vitro. The term “immunogen” refers to a compound, composition, or substance that is immunogenic as defined herein below.

The term ‘immunogenic composition” refers to a composition comprising an immunogen.

The term “MPE8” refers to an antibody described in Corti et al. [Corti, D., Bianchi, S., Vanzetta, F., Minola, A., Perez, L., Agatic, G., Lanzavecchia, A. Cross-neutralization of four paramyxoviruses by a human monoclonal antibody. Nature, 501(7467), 439-443 (2013)], which has a heavy chain variable domain comprising an amino acid sequence of SEQ ID NO:358 and a light chain variable domain comprising an amino acid sequence of SEQ ID NO:359.

The term “mutant” of a wild-type hMPV F protein, “mutant” of a hMPV F protein, “hMPV F protein mutant,” or “modified hMPV F protein” refers to a polypeptide that displays introduced mutations relative to a wild-type F protein and is immunogenic against the wild-type F protein.

The term “mutant” of a wild-type PIV1 F protein, “mutant” of a PIV1 F protein, “PIV1 F protein mutant,” or “modified PIV1 F protein” refers to a polypeptide that displays introduced mutations relative to a wild-type F protein and is immunogenic against the wild-type F protein.

The term “mutant” of a wild-type PIV3 F protein, “mutant” of a PIV3 F protein, “PIV3 F protein mutant,” or “modified PIV3 F protein” refers to a polypeptide that displays introduced mutations relative to a wild-type F protein and is immunogenic against the wild-type F protein.

The term “mutation” refers to deletion, addition, or substitution of amino acid residues in the amino acid sequence of a protein or polypeptide as compared to the amino acid sequence of a reference protein or polypeptide. Throughout the specification and claims, the substitution of an amino acid at one particular location in the protein sequence is referred to using a notation “(amino acid residue in wild type protein)(amino acid position)(amino acid residue in engineered protein)”. For example, a notation Y75 Arefers to a substitution of a tyrosine (Y) residue at the 75th position of the amino acid sequence of the reference protein by an alanine (A) residue (in a mutant of the reference protein). In cases where there is variation in the amino acid residue at the same position among different wild-type sequences, the amino acid code preceding the position number may be omitted in the notation, such as “75A.”

The term “native” or “wild-type” protein, sequence, or polypeptide refers to a naturally existing protein, sequence, or polypeptide that has not been artificially modified by selective mutations.

The term “pharmaceutically acceptable carriers” refers to a material or composition which, when combined with an active ingredient, is compatible with the active ingredient and does not cause toxic or otherwise unwanted reactions when administered to a subject, particularly a mammal. Examples of pharmaceutically acceptable carriers include solvents, surfactants, suspending agents, buffering agents, lubricating agents, emulsifiers, absorbents, dispersion media, coatings, and stabilizers.

The term “PIA174 mAb” refers to a PIV3 F protein prefusion specific antibody which has a heavy chain variable domain comprising an amino acid sequence of SEQ ID NO:364 and a light chain variable domain comprising an amino acid sequence of SEQ ID NO:365. The amino acid sequence of SEQ ID NO:364 comprises the heavy chain variable domains and constant domains of PIA174 mAb and the amino acid sequence of SEQ ID NO: 365 comprises the light chain variable domains and constant domains of PIA174 mAb. The heavy chain variable domain of PIA174 mAb has the amino acid sequence of SEQ ID NO:553. The light chain variable domain of PIA174 mAb has the amino acid sequence of SEQ ID NO:554.

The term “PIV1-8 mAb” (also referred to as hPIV1-8 mAb) refers to a PIV1 F protein prefusion specific antibody which has a heavy chain variable domain comprising an amino acid sequence of SEQ ID NO:362 and a light chain variable domain comprising an amino acid sequence of SEQ ID NO:363.

The term “prefusion-specific antibody” refers to an antibody that specifically binds to the F glycoprotein in a prefusion conformation, but does not bind to the F protein in a post-fusion conformation. Exemplary prefusion-specific antibodies include the MPE8, hMPV-2 and PIV1-8 antibody.

The term “prime-boost vaccination” refers to an immunotherapy regimen that includes administration of a first immunogenic composition (the primer vaccine) followed by administration of a second immunogenic composition (the booster vaccine) to a subject to induce an immune response. The primer vaccine and the booster vaccine typically contain the same immunogen and are presented in the same or similar format. However, they may also be presented in different formats, for example one in the form of a vector and the other in the form of a naked DNA plasmid. The skilled artisan will understand a suitable time interval between administration of the primer vaccine and the booster vaccine. Further, the primer vaccine, the booster vaccine, or both primer vaccine and the booster vaccine additionally include an adjuvant.

The term “prefusion conformation” refers to a structural conformation adopted by an F protein or mutant that can be specifically bound by a prefusion specific antibody such as for example MPE8 mAb for hMPV A, hMPV-2 mAb for hMPV B, PIV1-8 mAb for PIV1 and PIA174 mAb for PIV3.

The term “post-fusion conformation” refers to a structural conformation adopted by the F protein that is not specifically bound by MPE8 mAb, hMPV-2 mAb or PIV1-8. Native F protein adopts the post-fusion conformation subsequent to the fusion of the virus envelope with the host cellular membrane. F protein may also assume the post-fusion conformation outside the context of a fusion event, for example, under stress conditions such as heat and low osmolality, when extracted from a membrane, when expressed as an ectodomain, or upon storage.

The term “soluble protein” refers to a protein capable of dissolving in aqueous liquid and remaining dissolved. The solubility of a protein may change depending on the concentration of the protein in the water-based liquid, the buffering condition of the liquid, the concentration of other solutes in the liquid, for example salt and protein concentrations, and the temperature of the liquid.

The term “specifically bind,” in the context of the binding of an antibody to a given target molecule, refers to the binding of the antibody with the target molecule with higher affinity than its binding with other tested substances. For example, an antibody that specifically binds to the hMPV F protein in prefusion conformation is an antibody that binds hMPV F protein in prefusion conformation with higher affinity than it binds to the hMPV F protein in the post-fusion conformation.

The term “therapeutically effective amount” refers to the amount of agent that is sufficient to prevent, treat (including prophylaxis), reduce and/or ameliorate the symptoms and/or underlying causes of a disorder.

The term “vaccine” refers to a pharmaceutical composition comprising an immunogen that is capable of eliciting a prophylactic or therapeutic immune response in a subject. Typically, a vaccine elicits an antigen-specific immune response to an antigen of a pathogen, for example a viral pathogen.

A “variable region” of an antibody refers to the variable region of the antibody light chain or the variable region of the antibody heavy chain, either alone or in combination. As known in the art, the variable regions of the heavy and light chains each consist of four framework regions (FRs) connected by three complementarity determining regions (CDRs) also known as hypervariable regions, and contribute to the formation of the antigen binding site of antibodies. If variants of a subject variable region are desired, particularly with substitution in amino acid residues outside of a CDR region (e.g., in the framework region), appropriate amino acid substitution, preferably, conservative amino acid substitution, can be identified by comparing the subject variable region to the variable regions of other antibodies which contain CDR1 and CDR2 sequences in the same canonincal class as the subject variable region (Chothia and Lesk, J Mol Biol 196(4): 901-917, 1987).

In certain embodiments, definitive delineation of a CDR and identification of residues comprising the binding site of an antibody is accomplished by solving the structure of the antibody or solving the structure of the antibody-ligand complex. In certain embodiments, that can be accomplished by any of a variety of techniques known to those skilled in the art, such as X-ray crystallography. In certain embodiments, various methods of analysis can be employed to identify or approximate the CDR regions. In certain embodiments, various methods of analysis can be employed to identify or approximate the CDR regions. Examples of such methods include, but are not limited to, the Kabat definition, the Chothia definition, the AbM definition, the contact definition, the extended definition, and the conformational definition.

The Kabat definition is a standard for numbering the residues in an antibody and is typically used to identify CDR regions. See, e.g., Johnson & Wu, 2000, Nucleic Acids Res., 28: 214-8. The Chothia definition is similar to the Kabat definition, but the Chothia definition takes into account positions of certain structural loop regions. See, e.g., Chothia et al., 1986, J. Mol. Biol., 196: 901-17; Chothia et al., 1989, Nature, 342: 877-83. The extended definition is the combination of the Kabat and Chothia definitions. The AbM definition uses an integrated suite of computer programs produced by Oxford Molecular Group that model antibody structure. See, e.g., Martin et al., 1989, Proc Natl Acad Sci (USA), 86:9268-9272; “AbM™, A Computer Program for Modeling Variable Regions of Antibodies,” Oxford, UK; Oxford Molecular, Ltd. The AbM definition models the tertiary structure of an antibody from primary sequence using a combination of knowledge databases and ab initio methods, such as those described by Samudrala et al., 1999, “Ab Initio Protein Structure Prediction Using a Combined Hierarchical Approach,” in PROTEINS, Structure, Function and Genetics Suppl., 3:194-198. The contact definition is based on an analysis of the available complex crystal structures. See, e.g., MacCallum et al., 1996, J. Mol. Biol., 5:732-45. In another approach, referred to herein as the “conformational definition” of CDRs, the positions of the CDRs may be identified as the residues that make enthalpic contributions to antigen binding. See, e.g., Makabe et al., 2008, Journal of Biological Chemistry, 283:1156-1166. Still other CDR boundary definitions may not strictly follow one of the above approaches, but will nonetheless overlap with at least a portion of the Kabat CDRs, although they may be shortened or lengthened in light of prediction or experimental findings that particular residues or groups of residues do not significantly impact antigen binding. As used herein, a CDR may refer to CDRs defined by any approach known in the art, including combinations of approaches. The methods used herein may utilize CDRs defined according to any of these approaches. For any given embodiment containing more than one CDR, the CDRs may be defined in accordance with any one or more of Kabat, Chothia, extended, AbM, contact, or conformational definitions. Unless stated otherwise, the CDRs disclosed herein are defined in accordance with Kabat.

The term “vector” refers to a nucleic acid molecule capable of transporting or transferring a foreign nucleic acid molecule. The term encompasses both expression vectors and transcription vectors. The term “expression vector” refers to a vector capable of expressing the insert in the target cell, and generally contains control sequences, such as enhancer, promoter, and terminator sequences, that drive expression of the insert. The term “transcription vector” refers to a vector capable of being transcribed but not translated. Transcription vectors are used to amplify their insert. The foreign nucleic acid molecule is referred to as “insert” or “transgene.” A vector generally consists of an insert and a larger sequence that serves as the backbone of the vector. Based on the structure or origin of vectors, major types of vectors include plasmid vectors, cosmid vectors, phage vectors such as lambda phage, viral vectors such as adenovirus (Ad) vectors, and artificial chromosomes.

### B. hMPV Mutants

The present disclosure relates to hMPV F protein mutants, immunogenic compositions comprising the hMPV F protein mutants, methods for producing the hMPV F protein mutants, compositions comprising the hMPV F protein mutants, and nucleic acids that encode the hMPV F protein mutants.

### 1. EXEMPLARY EMBODIMENTS (E) OF THE INVENTION

Exemplary embodiments (E) of the invention provided herein include:
E1. A mutant of a wild-type hMPV F protein, which mutant comprises a F1 polypeptide and a F2 polypeptide, wherein the mutant comprises at least one amino acid mutation relative to the amino acid sequence of the wild-type hMPV F protein, and wherein the amino acid mutation is selected from the group consisting of:
(1) at least one engineered disulfide bond mutation;
(2) at least one cavity filling mutation;
(3) at least one proline substitution mutation;
(4) at least one glycine replacement mutation;
(5) a combination of at least one engineered disulfide mutation and at least one cavity filling mutation;
(6) a combination of at least one engineered disulfide mutation and at least one proline substitution mutation;
(7) a combination of at least one engineered disulfide mutation and a least one glycine replacement mutation;
(8) a combination of at least one engineered disulfide mutation, at least one cavity filling mutation and at least one proline substitution mutation;
(9) a combination of at least one engineered disulfide mutation, at least one cavity filling mutation, and a least one glycine replacement mutation;
(10) a combination of at least one engineered disulfide mutation, at least one proline substitution mutation and a least one glycine replacement mutation; and,
(11) a combination of at least one engineered disulfide mutation, at least one cavity filling mutation, at least one proline substitution mutation and a least one glycine replacement mutation.
E2. The mutant according E1, wherein the mutant comprises an engineered disulfide mutation selected from the group consisting of G366C and D454C, T411C and Q434C, 1137C and A159C, A140C and S149C, L141C and A159C, L141C and A161C, E146C and T160C, V148C and L158C and T150C and R156C.
E3. The mutant according to E1 or E2, wherein the engineered disulfide mutation is selected from the group consisting of T411C and Q434C, A140C and S149C, L141C and A161C, E146C and T160C and T150C and R156C.
E4. The mutant according to E2, wherein the engineered disulfide mutation is G366C and D454C.
E5. The mutant according to E2, wherein the engineered disulfide mutation is T411C and Q434C.
E6. The mutant according to E2, wherein the engineered disulfide mutation is 1137C and A159C.
E7. The mutant according E2, wherein the engineered disulfide mutation is A140C and S149C.
E8. The mutant according to E2, wherein the engineered disulfide mutation is L141C and A159C.
E9. The mutant according to E2, wherein the engineered disulfide mutation is L141C and A161C.
E10. The mutant according to E2, wherein the engineered disulfide mutation is E146C and T160C.
E11. The mutant according to E2, wherein the engineered disulfide mutation is V148C and L158C.
E12. The mutant according to E2, wherein the engineered disulfide mutation is T150C and R156C.
E13. The mutant according to E1, wherein the mutant comprises two engineered disulfide mutations selected from T411C and Q434C, A140C and S149C, L141C and A161C and E146C and T160C.
E14. The mutant according to E13, wherein the two engineered disulfide mutations are selected from T411C and Q434C and A140C and S149C.
E15. The mutant according to E13, wherein the two engineered disulfide mutations are selected from T411C and Q434C and L141C and A161C.
E16. The mutant according to any one of E1 to E15, wherein the mutant comprises a cavity filling mutation.
E17. The mutant according to E16, wherein the cavity filling mutation is selected from T49I, S149T, A159V, S2911, T365I and L473F.
E18. The mutant according to E17, wherein the cavity filling mutation is selected from T49I, S149T and T365I.
E19. The mutant according to E17, wherein the cavity filling mutation is T49I.
E20. The mutant according to E17, wherein the cavity filling mutation is S149T.
E21. The mutant according to E17, wherein the cavity filling mutation is A159V.
E22. The mutant according to E17, wherein the cavity filling mutation is S2911.
E23. The mutant according to E17, wherein the cavity filling mutation is T365I.
E24. The mutant according to E17, wherein the cavity filling mutation is L473F.
E25. The mutant according to E16, wherein the mutant comprises two cavity filling mutations selected from T49I, S149T, A159V, S2911, T365I and L473F.
E26. The mutant according to E25, wherein the mutant comprises two cavity filling mutations selected from T49I, S149T and T365I.
E27. The mutant according to E26, wherein the cavity filling mutations are T49I and S149T E28. The mutant according to E26, wherein the cavity filling mutations are T49I and T365I.
E29. The mutant according to E26, wherein the cavity filling mutations are S149T and T365I.
E30. The mutant according to E16, wherein the mutant comprises the cavity filling mutations T49I, S149T and T365I.
E31. The mutant according to any one of E1 to E30, wherein the mutant comprises a proline substitution mutation.
E32. The mutant according to E31, wherein the proline substitution mutation is selected from the group consisting of L66P, L110P, S132P, N145P, L187P, V449P and A459P.
E33. The mutant according to E32, wherein the proline substitution mutation is L66P.
E34. The mutant according to E32, wherein the proline substitution mutation is L11 OP.
E35. The mutant according to E32, wherein the proline substitution mutation is S132P.
E36. The mutant according to E32, wherein the proline substitution mutation is N145P.
E37. The mutant according to E32, wherein the proline substitution mutation is L187P.
E38. The mutant according to E32, wherein the proline substitution mutation is V449P.
E39. The mutant according to E32, wherein the proline substitution mutation is A459P.
E40. The mutant according to any one of E1 to E39, wherein the mutant comprises a glycine replacement mutation.
E41. The mutant according to E40, wherein the glycine replacement mutation is selected from the group consisting of G106A, G121A and G239A.
E42. The mutant according to any one of E2 to E41 wherein the mutant comprises the mutations Q100R and S101R.
E43. The mutant according to E1, wherein the mutant comprises the mutations selected from from the group consisting of:
(1) A140C and S149C,
(2) A140C, S149C, T411C and Q434C;
(3) A140C, S149C, T411C, Q434C and A459P;
(4) A140C, S149C, T411C, Q434C and T365I;
(5) A140C, S149C, T411C, Q434C and G239A;
(6) A140C, S149C, T411C, Q434C, A459P, G239A, T49I and T365I;
(7) T411C, Q434C, L141C and A161C;
(8) T411C, Q434C, L141C, A161C and A459P;
(9) T411C, Q434C, L141C, A161C and T49I;
(10) T411C, Q434C, L141C, A161C and T365I;
(11) T411C, Q434C, L141C, A161C and G239A;
(12) T411C, Q434C, L141C, A161C and S149T;
(13) T411C, Q434C, L141C, A161C, A459P, G239A, T49I, S149T and T365I, and,
(14) T411C, Q434C, E146C, T160C, A459P, G239A, T49I, S149T and T365I.
E44. The mutant according E1, wherein the mutant comprises the mutations selected from the group consisting of:
(1) T150C, R156C and A459P;
(2) T150C, R156C and T49I;
(3) T150C, R156C, T49I and A459P;
(4) A140C, S149C, T411C, and Q434C;
(5) L141C, A161C, T411C, and 434C;
(6) A140C, S149C, T411C, Q434C, and A459P;
(7) A140C, S149C, G239A, T411C and Q434C;
(8) L141C, A161C, T411C, Q434C and A459P;
(9) L141C, A161C, G239A, T411C and Q434C;
(10) T49I, T150C, R156C, G239A and A459P;
(11) A140C, S149C, G239A, T411C, Q434C and A459P;
(12)T49I, A140C, S149C, G239A, T4110, Q434C and A459P;
(13)T49I, A140C, G1490, G239A, T365I, T411, Q434C and A459P;
(14) L141C, A161C, G239A, T411C, Q434C and A459P;
(15) T49I, L141C, A1610C, G239A, T411C, Q434C and A459P;
(16) L141C, A161C, S149T, G239A, T411C, Q434C and A459P;
(17) T49I, L141C, A161C, S149T, G239A, T411C, Q434C and A459P;
(18) T49I, L1410C, A1610C, S149T, G239A, T365I, T4110, Q434C and A459P;
(19) T49I, S149T and A459P;
(20) A140C, S149C and A459P;
(21) T49I, A140C and S149C;
(22) T49I, A140C, S149C and A459P;
(23) T49I, L141C, A161C, T411C and Q434C;
(24) T49I, L141C, A161C, T411C, Q434C and A459P;
(25) L141C, A161C and S149T;
(26) L141C, A161C, S149T and A459P;
(27) T49I, L141C, A161C and S149T, and,
(28) T49I, L141C, A161C, S149T and A459P.
E45. The mutant according E1, wherein the mutant comprises the mutations selected from the group consisting of:
(1) L66P;
(2) L187P;
(4) A140C, S149C and L187P;
(5) T49I;
(6)T365I; and,
(7) T49I and T365I.
E46. The mutant according E1, wherein the mutant comprises the mutations selected from the group consisting of:
(1) L187P, Q100R and S101R; and,
(2) A140C, S149C, L187P, Q100R and S101R.
E47. The mutant according to E1 wherein
(a) the mutant comprises a cysteine (C) at position 140 (140C) and at position 149 (149C), and wherein the mutant comprises a F1 polypeptide and a F2 polypeptide selected from the group consisting of:
   (1) a F2 polypeptide comprising the amino acid sequence of SEQ ID NO:30 and a F1 polypeptide comprising the amino acid sequence of SEQ ID NO:29;
   (2) a F2 polypeptide comprising an amino acid sequence that is at least 97%, 98% or 99% identical to the amino acid sequence of SEQ ID NO:30 and a F1 polypeptide comprising an amino acid sequence that is at least 97%, 98%, or 99% identical to the amino acid sequence of SEQ ID NO:29 or;
(b) the mutant comprises a cysteine (C) at positions 411, 434, 140 and 149 and wherein the mutant comprises a F1 polypeptide and a F2 polypeptide selected from the group consisting of:
   (1) F2 polypeptide comprising the amino acid sequence of SEQ ID NO: 40 and a F1 polypeptide comprising the amino acid sequence of SEQ ID NO:39;
   (2) a F2 polypeptide comprising an amino acid sequence that is at least 97%, 98% or 99% identical to the amino acid sequence of SEQ ID NO:40 and a F1 polypeptide comprising an amino acid sequence that is at least 97%, 98%, or 99% identical to the amino acid sequence of SEQ ID NO:39, or
(c) the mutant comprises a cysteine (C) at positions 411, 434, 140 and 149 and a proline at position 459 and wherein the mutant comprises a F1 polypeptide and a F2 polypeptide selected from the group consisting of:
   (1) F2 polypeptide comprising the amino acid sequence of SEQ ID NO: 76 and a F1 polypeptide comprising the amino acid sequence of SEQ ID NO:75;
   (2) a F2 polypeptide comprising an amino acid sequence that is at least 97%, 98% or 99% identical to the amino acid sequence of SEQ ID NO:76 and a F1 polypeptide comprising an amino acid sequence that is at least 97%, 98%, or 99% identical to the amino acid sequence of SEQ ID NO:75, or
(d) the mutant comprises a cysteine (C) at positions 411, 434, 140 and 149 and an alanine at position 239 and wherein the mutant comprises a F1 polypeptide and a F2 polypeptide selected from the group consisting of:
   (1) F2 polypeptide comprising the amino acid sequence of SEQ ID NO: 80 and a F1 polypeptide comprising the amino acid sequence of SEQ ID NO:79;
   (2) a F2 polypeptide comprising an amino acid sequence that is at least 97%, 98% or 99% identical to the amino acid sequence of SEQ ID NO:80 and a F1 polypeptide comprising an amino acid sequence that is at least 97%, 98%, or 99% identical to the amino acid sequence of SEQ ID NO:79, or
(e) the mutant comprises a cysteine (C) at positions 411, 434, 140 and 149, a proline at position 459, an alanine at position 239 and an isoleucine at position 49 and 365 and wherein the mutant comprises a F1 polypeptide and a F2 polypeptide selected from the group consisting of:
   (1) F2 polypeptide comprising the amino acid sequence of SEQ ID NO: 94 and a F1 polypeptide comprising the amino acid sequence of SEQ ID NO:93;
   (2) a F2 polypeptide comprising an amino acid sequence that is at least 97%, 98% or 99% identical to the amino acid sequence of SEQ ID NO:94 and a F1 polypeptide comprising an amino acid sequence that is at least 97%, 98%, or 99% identical to the amino acid sequence of SEQ ID NO:93.
E48. The mutant according to any one of E1 to E47, wherein the F1 polypeptide lacks the entire cytoplasmic domain.
E49. The mutant according to any one of E1 to E48, wherein the F1 polypeptide lacks the cytoplasmic domain and a portion of or all entire transmembrane domain. Preferably, the F1 polypeptide lacks the cytoplasmic domain and the transmembrane domain.
E50. The mutant according to any one of E1 to E47, wherein the F1 polypeptide comprises the ectodomain, the transmembrane domain and the cytoplasmic domain. In a preferred embodiment, the mutant comprises the full length F1 polypeptide and the full length F2 polypeptide.
E51. The mutant according to any one of E1 to E49, wherein the mutant is linked to a trimerization domain. Preferably, the trimerization domain is a GCN4 leucine zipper or a phage T4 fibritin foldon.
E52. The mutant according to E51, wherein the trimerization domain is a phage T4 fibritin foldon.
E53. The mutant according to E52, wherein the trimerization domain is a phage T4 fibritin foldon of SEQ ID NO.7.
E54. The mutant according to any one of E50 to E53, wherein the trimerization domain is linked to the C-terminus of the F1 polypeptide.
E55. The mutant according to E54, wherein the trimerization domain is linked to the C-terminus of the F1 polypeptide via a linker.
E56. The mutant according to any one of E50 to E55, wherein the trimerization domain is linked to the C-terminus of the F1 polypeptide via a linker selected from the group consisting of GG, GS, GGGS or SAIG.
E57. The mutant according to E56, wherein the linker is GGGS.
E58. The mutant according to any one of E1 to E57, wherein the mutant is in the form of a trimer.
E59. The mutant according to any one of E1 to E58, wherein the mutant is in the prefusion conformation.
E60. The mutant according to any one of E1 to E59, wherein the mutant is in the prefusion conformation and specifically binds to an antibody (such as MPE8 mAb for hMPV A mutants or hMPV-2 mAb for hMPV B mutants) specific for the hMPV F ectodomain in the prefusion, but not postfusion, conformation.
E61. The mutant according to any one of E1 to E60, wherein the mutant is in the prefusion conformation and specifically binds to MPE8 mAb or hMPV-2 mAb as measured by ELISA, preferably as disclosed in the Examples.
E62. The mutant according to any one of E1 to E61, which has increased stability as compared with the corresponding wild-type hMPV F protein, wherein the stability is measured by binding of the mutant with antibody MPE8 (for hMPV A mutants) or hMPV-2 (for hMPV B mutants).
E63. The mutant of any one of E1 to E62 wherein the wild-type hMPV F protein is SEQ ID NO:1.
E64. The mutant of any one of E1 to E62 wherein the wild-type hMPV F protein is SEQ ID NO:2.
E65. The mutant of any one of E1 to E62 wherein the wild-type hMPV F protein is SEQ ID NO:3.
E66. The mutant of any one of E1 to E62 wherein the wild-type hMPV F protein is SEQ ID NO:4.
E67. The mutant of any one of E1 to E62 wherein the wild-type hMPV F protein is SEQ ID NO:5 or SEQ ID NO:6.
E68. The mutant of any one of E1 to E62 wherein the wild-type hMPV F protein is SEQ ID NO:99.
E69. The mutant of any one of E1 to E62 wherein the wild-type hMPV is of subtype A.
E70. The mutant of any one of E1 to E62 wherein the wild-type hMPV is of subtype B.
E71. The mutant of any one of E1 to E62 wherein the amino acid positions correspond to the amino acid sequence of a reference of SEQ ID NO:1.
E72. The mutant of any one of E1 to E62 wherein the amino acid positions correspond to the amino acid sequence of a reference of SEQ ID NO:4.
E73. A nucleic acid comprising at least one coding sequence encoding at least one mutant of a wild-type hMPV F protein according to any one of embodiments E1-E72, preferably E50, or an immunogenic fragment or immunogenic variant thereof, wherein the nucleic acid comprises at least one heterologous untranslated region (UTR).
E74. A nucleic acid according to any one of the preceding embodiments, wherein the at least one heterologous untranslated region is selected from at least one heterologous 5′-UTR and/or at least one heterologous 3′-UTR.
E75. A nucleic acid according to any one of the preceding embodiments, wherein the at least one heterologous 3′-UTR comprises or consists of a nucleic acid sequence having at least, at most, exactly, or between any two of 99%, 98%, 97%, 96%, 95%, 90%, 85%, or 80% identity to CΨCGAGCΨGGΨACΨGCAΨGCA CGCAAΨGCΨA GCΨGCCCCΨΨΨCCCGΨCCΨG GGΨACCCCGA GΨCΨCCCCCG ACCΨCGGGΨC CCAGGΨAΨGC WCCCACCWCC ACCWGCCCCA CWCACCACCW CWGCWAGWWC CAGACACCWC CCAAGCACGC AGCAAWGCAG CYCAAAACGC WWAGCCWAGC CACACCCCCA CGGGAAACAG CAGWGAWΨAA CCΨΨΨAGCAA WAAACGAAAG ΨΨΨAACΨAAG CΨAΨACΨAAC CCCAGGGΨΨG GΨCAAΨΨΨCG ΨGCCAGCCAC ACCCYGGAGC WAGC.
E76. A nucleic acid according to any one of the preceding embodiments, wherein the at least one heterologous 5′-UTR comprises or consists of a nucleic acid sequence having at least, at most, exactly, or between any two of 99%, 98%, 97%, 96%, 95%, 90%, 85%, or 80% identity to GAAΨAAAC ΨAGΨAΨΨCΨΨCYGGYCCCCA CAGACYCAGA GAGAACCCGC CACC.
E77. A nucleic acid according to any one of the preceding embodiments, wherein the nucleic acid comprises at least one poly(A) sequence, preferably comprising 30 to 200 adenosine nucleotides and/or at least one poly(C) sequence, preferably comprising 10 to 40 cytosine nucleotides.
E78. A nucleic acid according to any one of the preceding embodiments, wherein the nucleic acid is a DNA or an RNA.
E79. A nucleic acid according to any one of the preceding embodiments, wherein the nucleic acid is a coding RNA.
E80. A nucleic acid according to E79, wherein the coding RNA is an mRNA, a self-replicating RNA, a circular RNA, or a replicon RNA.
E81. A nucleic acid according to any one of the preceding embodiments, wherein the nucleic acid, preferably the coding RNA, is an mRNA.
E82. A nucleic acid according to E81, wherein the mRNA is not a replicon RNA or a self-replicating RNA.
E83. A nucleic acid according to any one of the preceding embodiments E80-E82, wherein the mRNA comprises at least one poly(A) sequence comprising 30 to 200 adenosine nucleotides and the 3′ terminal nucleotide is an adenosine.
E84. A nucleic acid according to any one of the preceding embodiments E78-E83, wherein the RNA, preferably the coding RNA, comprises a 5′-cap structure, preferably m7G, capO, cap1, cap2, a modified capO or a modified cap1 structure, preferably a 5′-cap1 structure.
E85. A nucleic acid according to any one of the preceding embodiments E78-E84, wherein the RNA is codon-optimized.
E86. A nucleic acid according to any one of the preceding embodiments E78-E85, wherein the RNA comprises a chemically modified nucleotide.
E87. A nucleic acid according to any one of the preceding embodiments E78-E86, wherein the RNA comprises 1-methylpseudouridine substitution. Preferably, all the uridines of the RNA are replaced by 1-methylpseudouridine.
E88. A nucleic acid according to any one of the preceding embodiments E78-E87, wherein the RNA is a purified RNA, preferably an RNA that has been purified by RP-HPLC and/or TFF.
E89. A nucleic according to any one of the preceding embodiments E78 to E88 wherein the RNA comprises the nucleic acid sequence of any of SEQ ID NO:391, SEQ ID NO:393, SEQ ID NO:395; SEQ ID NO:397, SEQ ID NO:399, SEQ ID NO:512, SEQ ID NO:514, SEQ ID NO:516, SEQ ID NO:518, SEQ ID NO:520, SEQ ID NO:522, SEQ ID NO:524 and SEQ ID NO:526.
E90. A composition comprising at least one nucleic acid according to any one of the preceding embodiments E73-E88.
E91. A composition comprising at least one nucleic acid according to any one of the preceding embodiments E73-E889, wherein the composition comprises at least one pharmaceutically acceptable carrier.
E92. A composition comprising at least one nucleic acid according to any one of the preceding embodiments E73-E889, wherein the composition is a multivalent composition comprising a plurality or at least more than one of the nucleic acid according to any one of E73 to E89.
E93. A composition comprising at least one nucleic acid according to any one of the preceding embodiments E73-E889, wherein the composition comprises RNA with an RNA integrity of 70% or more.
E94. A composition comprising at least one nucleic acid according to any one of the preceding embodiments E73-E889, wherein the composition comprises RNA with a capping degree of 70% or more, preferably wherein at least 70%, 80%, or 90% of the mRNA species comprise a Cap1 structure.
E95. A composition comprising at least one nucleic acid according to any one of the preceding embodiments E73-E8889, wherein the at least one nucleic acid is complexed or associated with or at least partially complexed or partially associated with one or more cationic or polycationic compound, preferably cationic or polycationic polymer, cationic or polycationic polysaccharide, cationic or polycationic lipid, cationic or polycationic protein, cationic or polycationic peptide, or any combinations thereof.
E96. A composition comprising at least one nucleic acid according to any one of the preceding embodiments E73-E889, wherein the at least one nucleic acid is complexed or associated with one or more lipids or lipid-based carriers, thereby forming liposomes, lipid nanoparticles (LNP), lipoplexes, and/or nanoliposomes, preferably encapsulating the at least one nucleic acid.
E97. A composition comprising at least one nucleic acid according to any one of the preceding embodiments E73-E889, wherein the at least one nucleic acid is complexed with one or more lipids thereby forming lipid nanoparticles.
E98. A composition according to any one of the preceding embodiments E96-E97, wherein the LNP comprises a cationic lipid according to formula III-3:

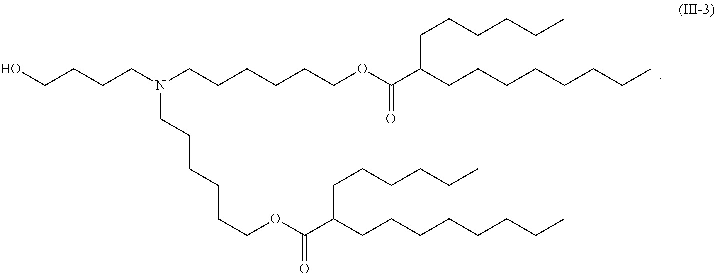 [SMILES: CCCCCCCCC(CCCCCC)C(=O)OCCCCCCN(CCCCO)CCCCCCOC(=O)C(CCCCCC)CCCCCCCC]

E99. A composition according to any one of the preceding embodiments E9696-E9898, wherein the LNP comprises a PEG lipid of formula (IVa):

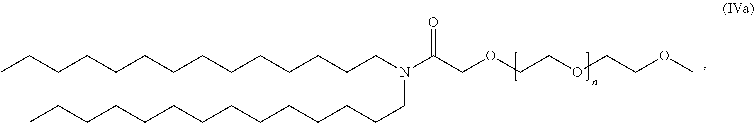

E100. A composition according to embodiment E9999, wherein n has a mean value ranging from 30 to 60, preferably wherein n has a mean value of about 45, 46, 47, 48, 49, 50, 51, 52, 53, 54, most preferably wherein n has a mean value of 49 or 45.
E101. A composition according to any one of the preceding embodiments E9696-E100100, wherein the LNP comprises a PEG lipid of formula (IVa):

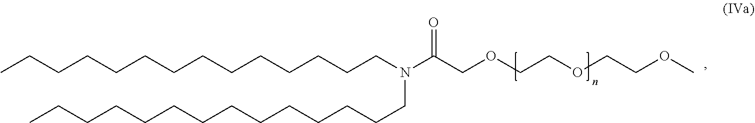

wherein n is an integer selected such that the average molecular weight of the PEG lipid is about 2500 g/mol.
E102. A composition according to any one of the preceding embodiments E9696-E101101, wherein the LNP comprises one or more neutral lipids and/or one or more steroid or steroid analogues.
E103. A composition according to any one of the preceding embodiments E9696-E1011, wherein the neutral lipid is 1,2-distearoyl-sn-glycero-3-phosphocholine (DSPC), preferably wherein the molar ratio of the cationic lipid to DSPC is in the range from about 2:1 to about 8:1.
E104. A composition according to any one of the preceding embodiments E9696-E103, wherein the steroid is cholesterol, preferably wherein the molar ratio of the cationic lipid to cholesterol is in the range from about 2:1 to about 1:1.
E105. A composition according to any one of the preceding embodiments E9696-104, wherein the LNP comprises (i) at least one cationic lipid, preferably a lipid of formula (III), more preferably lipid III-3; (ii) at least one neutral lipid, preferably 1,2-distearoyl-sn-glycero-3-phosphocholine (DSPC); (iii) at least one steroid or steroid analogue, preferably cholesterol; and (iv) at least one polymer conjugated lipid, preferably a PEG-lipid derived from formula (IVa, with n=49), wherein (i) to (iv) are in a molar ratio of about 20-60% cationic lipid, 5-25% neutral lipid, 25-55% sterol, and 0.5-15% PEG-lipid.
E106. A composition according to any one of the preceding embodiments E9696-E106, wherein the LNP comprises (i) at least one cationic lipid, preferably a lipid of formula (III), more preferably lipid III-3; (ii) at least one neutral lipid, preferably 1,2-distearoyl-sn-glycero-3-phosphocholine (DSPC); (iii) at least one steroid or steroid analogue, preferably cholesterol; and (iv) at least one polymer conjugated lipid, preferably a PEG-lipid derived from formula (IVa, with n=45), wherein (i) to (iv) are in a molar ratio of about 20-60% cationic lipid, 5-25% neutral lipid, 25-55% sterol, and 0.5-15% PEG-lipid.
E107. A composition according to any one of the preceding embodiments E105-E106, wherein (i) to (iv) are in a molar ratio of about 50:10:38.5:1.5, preferably 47.5:10:40.8:1.7 or more preferably 47.4:10:40.9:1.7.
E108. A composition according to any one of the preceding embodiments E9696-E107, wherein the nucleic acid is RNA and the composition comprises less than about 20% free (non complexed or non-encapsulated) RNA, preferably less than about 15% free RNA, more preferably less than about 10% free RNA.
E109. A composition according to any one of the preceding embodiments E966-E108, wherein the wt/wt ratio of lipid to nucleic acid is from about 10:1 to about 60:1, preferably from about 20:1 to about 30:1, for example about 25:1.
E110. A composition according to any one of the preceding embodiments E966-E109, wherein the n/p ratio of the LNPs encapsulating the nucleic acid is in a range from about 1 to about 10, preferably in a range from about 5 to about 7, more preferably about 6.
E111. A composition according to any one of the preceding embodiments E966-E110, wherein the composition has a polydispersity index (PDI) value of less than about 0.4, preferably of less than about 0.3, more preferably of less than about 0.2, most preferably of less than about 0.1.
E112. A composition according to any one of the preceding embodiments E966-E111, wherein the LNPs have a Z-average size in a range of about 60 nm to about 120 nm, preferably less than about 120 nm, more preferably less than about 100 nm, most preferably less than about 80 nm.
E113. A composition according to any one of the preceding embodiments E966-E112, wherein the LNPs comprise less than about 10%, 9%, 8%, 7%, 6%, 5%, 4%, 3%, 2%, 1% LNPs that have a particle size exceeding about 500 nm.
E114. A composition according to any one of the preceding embodiments E966-E113, wherein the LNPs comprise less than about 10%, 9%, 8%, 7%, 6%, 5%, 4%, 3%, 2%, 1% LNPs that have a particle size smaller than about 20 nm.
E115. A composition according to any one of the preceding embodiments E966-E114, wherein the LNP comprises (i) at least one cationic lipid; (ii) at least one neutral lipid; (iii) at least one steroid or steroid analogue; and (iv) at least one PEG-lipid, wherein (i) to (iv) are in a molar ratio of about 20-60% cationic lipid, 5-25% neutral lipid, 25-55% sterol, and 0.5-15% PEG-lipid.
E116. A composition according to any one of the preceding embodiments E966-E115, wherein the LNP comprises (i) at least one cationic lipid according to formula III-3; (ii) DSPC; (iii) cholesterol; and (iv) a PEG-lipid, according to formula IVa, wherein (i) to (iv) are in a molar ratio of about 20-60% cationic lipid, 5-25% neutral lipid, 25-55% sterol, and 0.5-15% PEG-lipid.
E117. A composition according to any one of the preceding embodiments E9090-E116, wherein the composition is a lyophilized composition.
E118. An immunogenic composition comprising a mutant according to any one of E1 to E72, a nucleic acid according to any one of E73 to E89 or a composition according to any one of E90 to E117.
E119. An immunogenic composition according to E118 comprising a mutant according to any one of E1 to E72, a nucleic acid according to any one of E73 to E89 or a composition according to any one of E90 to E117 wherein the wild-type hMPV F protein is of subtype A and, a mutant according to any one of E1 to E72, a nucleic acid according to any one of E73 to E89 or a composition according to any one of E90 to E117 wherein the wild-type hMPV F protein is of subtype B.
E120. An immunogenic composition according to any one of embodiments E118-E119, further comprising a PIV1 antigen selected from the group consisting of a mutant of a wild-type PIV1 F protein and a nucleic acid encoding a mutant of a wild-type PIV1 F protein.
E121. An immunogenic composition according to embodiment E120, wherein the PIV1 antigen is a mutant of a wild-type PIV1 F protein.
E122. An immunogenic composition according to embodiment E121, wherein the PIV1 antigen is a mutant of a wild-type PIV1 F protein from the present disclosure, preferably from any of E1 to E56 of section C of the present disclosure.
E123. An immunogenic composition according to embodiment E121, wherein the PIV1 antigen comprises a nucleic acid encoding a mutant of a wild-type PIV1 F protein.
E124. An immunogenic composition according to embodiment E123, wherein the PIV1 antigen comprises a nucleic acid encoding a mutant of a wild-type PIV1 F protein from the present disclosure, preferably from any of E57 to E73 of section C of the present disclosure.
E125. An immunogenic composition according to any one of claims E1188 to E1244, further comprising PIV3 antigen selected from the group consisting of a mutant of a wild-type PIV3 F protein and a nucleic acid encoding a mutant of a wild-type PIV3 F protein.
E126. An immunogenic composition according to embodiment E125, wherein the PIV3 antigen is a mutant of a wild-type PIV3 F protein.
E127. An immunogenic composition according to embodiment E125, wherein the PIV3 antigen is a mutant of a wild-type PIV3 F protein from the present disclosure, preferably from any of E1 to E52 of section D of the present disclosure.
E128. An immunogenic composition according to embodiment E125, wherein the PIV3 antigen is a mutant of a wild-type PIV3 F protein as disclosed in WO2018081289 or WO22207839.
E129. An immunogenic composition according to embodiment E125, wherein the PIV3 antigen comprises a nucleic acid encoding a mutant of a wild-type PIV3 F protein.
E130. An immunogenic composition according to embodiment E125, wherein the PIV3 antigen comprises a nucleic acid encoding a mutant of a wild-type PIV3 F protein from the present disclosure, preferably from any of E53 to E69 of section D of the present disclosure.
E131. An immunogenic composition according to embodiment E125, wherein the PIV3 antigen comprises a nucleic acid encoding a mutant of a wild-type PIV3 F protein as disclosed in WO2018081289 or WO2022207839.
E132. An immunogenic composition according to any one of E1188 to 131, further comprising an RSV antigen selected from the group consisting of a mutant of a wild-type RSV F protein of subtype A and a nucleic acid encoding a mutant of a wild-type RSV F protein of subtype A.
E133. An immunogenic composition according to embodiment E132, wherein the RSV antigen is a mutant of a wild-type RSV F protein of subtype A.
E134. An immunogenic composition according to embodiment E132, wherein the RSV antigen is a nucleic acid encoding a mutant of a wild-type RSV F protein of subtype A.
E135. An immunogenic composition according to embodiments E132-134, wherein the mutant of a wild-type RSV F protein of subtype A is disclosed in one of WO2009/079796, WO2010/149745, WO2011/008974, WO2014/160463, WO2014/174018, WO2014/202570, WO2015/013551, WO2015/177312, WO2017/005848, WO2017/174564, WO2017/005844, WO2017/109629, WO2022/002894 and WO2018/109220.
E136. An immunogenic composition according to any one of embodiments E1188 to E135, further comprising an RSV antigen selected from the group consisting of a mutant of a wild-type RSV F protein of subtype B and a nucleic acid encoding a mutant of a wild-type RSV F protein of subtype B.
E137. An immunogenic composition according to embodiment E136, wherein the RSV antigen is a mutant of a wild-type RSV F protein of subtype B.
E138. An immunogenic composition according to embodiment E136, wherein the RSV antigen comprises a nucleic acid encoding a mutant of a wild-type RSV F protein of subtype B.
E139. An immunogenic composition according to embodiment E137 or E138, wherein the mutant of a wild-type RSV F protein of subtype B is disclosed in one of WO2009/079796, WO2010/149745, WO2011/008974, WO2014/160463, WO2014/174018, WO2014/202570, WO2015/013551, WO2015/177312, WO2017/005848, WO2017/174564, WO2017/005844, WO2017/109629, WO2022/002894 and WO2018/109220.

### 2. hMPV F Protein Mutants

In some aspects, the present invention provides mutants of wild-type hMPV F proteins, wherein the mutants display introduced mutations in the amino acid sequence relative to the amino acid sequence of the corresponding wild-type hMPV F protein and are immunogenic against the wild-type hMPV F protein in the prefusion conformation or against a virus comprising the wild-type F protein. In certain embodiments, the hMPV F mutants possess certain beneficial characteristics, such as increased immunogenic properties or improved stability in the prefusion conformation of the mutants or prefusion trimeric conformation of the mutant, as compared to the corresponding wild-type F protein. In still other embodiments, the present disclosure provides hMPV F mutants that display one or more introduced mutations as described herein and bind to a prefusion specific antibody selected from MPE8 mAb (for hMPV A mutants) or hMPV-2 mAb (for hMPV B mutants).

The introduced amino acid mutations in the hMPV F protein mutants include amino acid substitutions, deletions, or additions. In some embodiments, the only mutations in the amino acid sequence of the mutants are amino acid substitutions relative to a wild-type hMPV F protein.

The amino acid sequence of a large number of native hMPV F proteins from different hMPV subtypes, as well as nucleic acid sequences encoding such proteins, is known in the art. For example, the sequence of several subtype A and B hMPV F0 precursor proteins are set forth in SEQ ID NOs:1 to 6 and 99.

The native hMPV F protein exhibits remarkable sequence conservation across hMPV subtypes. For example, hMPV subtypes A and B consensus sequences share about 94% sequence identity across the F0 precursor molecule. Nearly all identified hMPV F0 precursor sequences consist of 539 amino acids in length, with minor differences in length. Sequence identity across various native hMPV F proteins is known in the art (see, for example, Yang et al, Virology Journal 2009, 6:138).

In view of the substantial conservation of hMPV F protein sequences, a person of ordinary skill in the art can easily compare amino acid positions between different native hMPV F protein sequences to identify corresponding hMPV F protein amino acid positions between different hMPV strains and subtypes. For example, across nearly all identified native hMPV F0 precursor proteins, the protease cleavage site falls in the same amino acid positions. Thus, the conservation of native hMPV F protein sequences across strains and subtypes allows use of a reference hMPV F protein sequence for comparison of amino acids at particular positions in the hMPV F protein. For the purposes of this disclosure (unless context indicates otherwise), the hMPV F protein amino acid positions are given with reference to the sequence of the F0 precursor polypeptide set forth in SEQ ID NO: 1 (the amino acid sequence of the full length native F precursor polypeptide of the hMPV A2b strain; corresponding to Genbank Identifier ACJ53569.1 (amino acids) and EU857558.1 (nucleotides).

For the purposes of this disclosure (unless context indicates otherwise), the hMPV A F protein amino acid positions are given with reference to the sequence of the F0 precursor polypeptide set forth in SEQ ID NO: 1 (the amino acid sequence of the full length native F precursor polypeptide of the hMPV A2b strain; corresponding to Genbank Identifier ACJ53569.1 (amino acids) and EU857558.1 (nucleotides).

For the purposes of this disclosure (unless context indicates otherwise), the hMPV B F protein amino acid positions are given with reference to the sequence of the F0 precursor polypeptide set forth in SEQ ID NO: 4 (the amino acid sequence of the full length consensus F precursor polypeptide of the hMPV B strain).

The consensus sequence for hMPV B was obtained as follows: Whole genome sequences for hMPV B were downloaded from NCBI's GenBank database as GenBank file format. Fusion protein gene sequences were filtered by sequence length to only include complete coding DNA sequence features. Translated fusion protein sequences were then parsed from GenBank file and saved as FASTA file. Muscle v5 was used to perform multiple sequence alignment of collected sequences. A Position specific score matrices (PSSMs) was generated to summarize the alignment information. For each column in the alignment, the number of each amino acid letters is counted and totaled. The consensus sequence at each position was calculated as the most common amino acid type in PSSM table. The final consensus sequence was then extracted and saved as FASTA file.

However, it should be noted, and one of skill in the art will understand, that different hMPV F0 sequences may have different numbering systems, for example, if there are additional amino acid residues added or removed as compared to SEQ ID NO:1. As such, it is to be understood that when specific amino acid residues are referred to by their number, the description is not limited to only amino acids located at precisely that numbered position when counting from the beginning of a given amino acid sequence, but rather that the equivalent/corresponding amino acid residue in any and all hMPV F sequences is intended even if that residue is not at the same precise numbered position, for example if the hMPV sequence is shorter or longer than SEQ ID NO:1, or has insertions or deletions as compared to SEQ ID NO: 1.

2-1. Structure of the hMPV F Protein Mutants

The hMPV F protein mutants provided by the present disclosure comprise a F1 polypeptide and a F2 polypeptide. In several embodiments, the mutants further comprise a trimerization domain. In some embodiments, either the F1 polypeptide or the F2 polypeptide includes at least one introduced modification (e.g., amino acid substitution) as described in detail herein below. In some other embodiments, each of the F1 polypeptide and F2 polypeptide includes at least one introduced modification (e.g., amino acid substitution) as described in detail herein below.

2-1(a). F1 Polypeptide and F2 Polypeptide of the hMPV F Mutants

In some embodiments, the mutants are in the mature form of the hMPV F protein, which comprises two separate polypeptide chains, namely the F1 polypeptide and F2 polypeptide.

The F1 polypeptide chain of the mutant may be of the same length as the full length F1 polypeptide of the corresponding wild-type hMPV F protein; however, it may also have deletions, such as deletions of 1 up to 36 amino acid residues from the C-terminus of the full-length F1 polypeptide. A full-length F1 polypeptide of the hMPV F mutants corresponds to amino acid positions 103-539 of the native hMPV F0 precursor, and includes (from N- to C-terminus) an extracellular region (residues 103 to 489), a transmembrane domain (residues 490-514), and a cytoplasmic domain (residues 515-539). It should be noted that amino acid residues 490 onwards in a native F1 polypeptide sequence are optional sequences in a F1 polypeptide of the hMPV F mutants provided herein, and therefore may be absent from the F1 polypeptide of the mutant.

In some embodiments, the F1 polypeptide of the hMPV F mutants lacks the entire cytoplasmic domain. In other embodiments, the F1 polypeptide lacks the cytoplasmic domain and a portion of or all entire transmembrane domain. In some specific embodiments, the mutant comprises a F1 polypeptide wherein the amino acid residues from position 490 through 539 are absent. Typically, for mutants that are linked to trimerization domain, such as a foldon, amino acids 490 through 539 can be absent. Thus, in some specific embodiment, amino acid residues 490 through 539 are absent from the F1 polypeptide of the mutant. In still other specific embodiments, the F1 polypeptide of the hMPV F mutants comprises or consists of amino acid residues 103-489 of a native F0 polypeptide sequence, such as any of the F0 precursor sequence set forth in SEQ ID Nos: 1 to 6 and 99.

On the other hand, the F1 polypeptide of the hMPV F mutant may include a C-terminal linkage to a trimerization domain, such as a foldon. Many of the sequences of the hMPV F mutants disclosed herein include a sequence of a PreScission cleavage site and Strep Tag II that are not essential for the function of the hMPV F protein, such as for induction of an immune response. A person skilled in the art will recognize such sequences, and when appropriate, understand that these sequences are not included in a disclosed hMPV F mutant.

In the hMPV F mutants provided by the present disclosure, the F2 polypeptide chain may be of the same length as the full-length F2 polypeptide of the corresponding wild-type hMPV F protein; it may also have deletions, such as deletions of 1, 2, 3, 4, 5, 6, 7, or 8 amino acid residues from the N-terminus or C-terminus of the F2 polypeptide.

The mutant in F0 form (i.e., a single chain polypeptide comprising the F2 polypeptide joined to the F1 polypeptide) or F1-F2 heterodimer form may form a protomer. The mutant may also be in the form of a trimer, which comprises three of the same protomer. Further, the mutants may be glycosylated proteins (i.e., glycoproteins) or non-glycosylated proteins. The mutant in F0 form may include, or may lack, the signal peptide sequence.

The F1 polypeptide and F2 polypeptide of the hMPV F protein mutants to which one or more mutations are introduced can be from any wild-type hMPV F proteins known in the art or discovered in the future, including, without limitations, the F protein amino acid sequence of hMPV subtype A, and subtype B strains, or any other subtype. In some embodiments, the hMPV F mutant comprises a F1 and/or a F2 polypeptide from a hMPV A virus, for example, a F1 and/or F2 polypeptide from a known hMPV F0 precursor protein such for example those set forth in any one of SEQ ID NOs: 1 to 3 to which one or more mutations are introduced. In some other embodiments, the hMPV F mutant comprises a F1 and/or a F2 polypeptide from a hMPV B virus, for example, a F1 and/or F2 polypeptide from a known hMPV F0 precursor protein such as those set forth in any one of SEQ ID NOs: 4 to 6 or 99 to which one or more mutations are introduced.

In some embodiments, the hMPV F protein mutants comprise a F1-polypeptide, a F2 polypeptide, and one or more introduced amino acid mutations as described herein below, wherein the F1 polypeptide comprises 350 consecutive amino acids and is at least 90, 95, 98, or 99 percent identical to amino acids 103-489 of any of the sequence of SEQ ID NO:1 to 3, wherein the F2 polypeptide comprises 70 consecutive amino acids and is at least 90, 95, 98, or 99 percent identical to amino acids 21-102 of any of the sequence of SEQ ID NO:1 to 3 and wherein hMPV F protein mutant is stabilized in prefusion trimer conformation, whether as monomer or trimer.

In some embodiments, the hMPV F protein mutants comprise a F1-polypeptide, a F2 polypeptide, and one or more introduced amino acid mutations as described herein below, wherein the F1 polypeptide comprises 350 consecutive amino acids and is at least 90, 95, 98, or 99 percent identical to amino acids 103-489 of any of the sequence of SEQ ID NO:4 to 6 or 99, wherein the F2 polypeptide comprises 70 consecutive amino acids and is at least 90, 95, 98, or 99 percent identical to amino acids 21-102 of any of the sequence of SEQ ID NO:4 to 6 or 99 and wherein hMPV F protein mutant is stabilized in prefusion trimer conformation, whether as monomer or trimer.

2-1(b) Trimerization Domains

In several embodiments, the hMPV F mutant provided by the present disclosure is linked to a trimerization domain. In some embodiments, the trimerization domain promotes the formation of trimer of three F1/F2 heterodimers.

Several exogenous trimerization domains that promote formation of stable trimers of soluble proteins are known in the art. Non limiting examples of such trimerization domains that can be linked to a mutant provided by the present disclosure include: (1) the GCN4 leucine zipper (Harbury et al. 1993 Science 262: 1401-1407); (2) the trimerization motif from the lung surfactant protein (Hoppe et al. 1994 FEB S Lett 344: 191-195); (3) collagen (McAlinden et al. 2003 Biol Chem 278:42200-42207); and (4) the phage T4 fibritin foldon (Miroshnikov et al. 1998 Protein Eng 11:329-414).

Typically, the trimerization domain is positioned C-terminal to the F1 polypeptide. It may join directly to the F1 polypeptide chain. Optionally, the multimerization domain is connected to the F1 polypeptide via a linker, such as an amino acid linker, for example the sequence GG, GS, GGGS, or SAIG. The linker can also be a longer linker (for example, including the repeat sequence GG). A preferred linker is GGGS. Numerous conformationally neutral linkers are known in the art that can be used in the mutants provided by the present disclosure. In some embodiments, the F mutant comprising a foldon domain include a protease cleavage site for removing the foldon domain from the F1 polypeptide, such as a thrombin site between the F1 polypeptide and the foldon domain.

In some embodiments, a foldon domain is linked to a F mutant at the C-terminus of F1 polypeptide. In specific embodiments, the foldon domain is a T4 fibritin foldon domain, such as the amino acid sequence GYIPEAPRDGQAYVRKDGEWVLLSTFL (SEQ ID NO: 7).

2-2. Introduced Mutations in the hMPV F Protein Mutants

The hMPV F mutants provided by the present disclosure comprise a F1 polypeptide and a F2 polypeptide, wherein (1) either the F1 polypeptide or (2) the F2 polypeptide, or (3) both the F1 polypeptide and F2 polypeptide include one or more introduced amino acid mutations relative to the amino acid sequence of the corresponding native F protein. The introduction of such amino acid mutations in the hMPV F mutants confers a beneficial property to the mutants, such as enhanced immunogenicity, improved stability, improved expression or formation or improved stability of certain desired physical form or conformation of the mutants. Such introduced amino acid mutations are referred to as “engineered disulfide bond mutations,” “cavity filling mutations”, “proline substitution mutations” or “glycine replacement mutation”, and are described in detail herein below. hMPV F mutants that include any additional mutations are also encompassed by the invention so long as the immunogenic property of the mutants is not substantially adversely affected by the additional mutations.

2-2(a) Engineered Disulfide Bond Mutations

In some embodiments, hMPV F mutants provided by the present disclosure include one or more engineered disulfide bond mutations. The term “engineered disulfide bond mutation” refers to mutation of a pair of amino acid residues in a wild-type hMPV F protein to a pair of cysteine residues. The introduced pair of cysteine residues allows for formation of a disulfide bond between the introduced cysteine residues, which disulfide bond serves to stabilize the protein's conformation or oligomeric state, such as prefusion conformation. For stabilizing the prefusion conformation of the mutant, the residue pairs for mutation to cysteine should be in close proximity in the prefusion conformation but distant in the post-fusion conformation. Preferably, the distance between the pair of residues (e.g. the beta carbons) is less than 8 Ain a prefusion conformation, but more than 20 Ain a post-fusion conformation.

In some embodiments, the hMPV F protein mutants comprise only one engineered disulfide mutation (“single engineered disulfide mutation”). In some other embodiments, the hMPV F protein mutants comprise at least two engineered disulfide mutations, wherein each pair of the cysteine residues of the engineered disulfide mutations are appropriately positioned when hMPV F protein mutant is in prefusion conformation (“double engineered disulfide mutation”).

In some specific embodiments, the present disclosure provides a hMPV F mutant comprising at least one engineered disulfide bond mutation, wherein the mutant comprises the same introduced mutations that are in any of the exemplary mutants provided in Tables 9, 13-16, 19 and 23-28. The exemplary hMPV F mutants provided in Tables 9, 13-16, 19 and 23-28 are based on the same native F0 sequence of hMPV A strain TN/95/3-54 (SEQ ID NO:128) or the consensus F0 sequence of hMPV B strain (SEQ ID NO:129), depending on whether the mutants is a hMPV A or hMPV B F protein mutant. The same introduced mutations in each of the mutants can be made to a native F0 polypeptide sequence of any other hMPV subtype or strain to arrive at different hMPV F mutants, such as a native F0 polypeptide sequence set forth in any of the SEQ ID NOs: 1 to 6 and 99 or from any other hMPV A or B strain. hMPV F mutants that are based on a native F0 polypeptide sequence of any other hMPV subtype or strain and comprise any of the engineered disulfide mutations are also within the scope of the invention. In some particular embodiments, a hMPV F protein mutant comprises at least one engineered disulfide mutation selected from the group consisting of: 366C and 454C, 411C and 434C, 137C and 159C, 140C and 149C, 141C and 159C, 141C and 161C, 146C and 160C,148C and 158C and 150C and 156C, such as G366C and D454C, T411C and Q434C, 1137C and A159C, A140C and S149C, L141C and A159C, L141C and A161C, E146C and T160C, V148C and L158C and T150C and R156C.

2-2(b) Cavity Filling Mutations.

In other embodiments, the present disclosure provides hMPV F mutants that comprise one or more cavity filling mutations. The term “cavity filling mutation” refers to the substitution of an amino acid residue in the wild-type hMPV F protein by an amino acid that is expected to fill an internal cavity of the mature hMPV F protein. In one application, such cavity-filling mutations contribute to stabilizing the prefusion conformation of a hMPV F protein mutant. For example, the amino acids to be replaced for cavity-filling mutations typically include small aliphatic (e.g. Gly, Ala, and Val) or small polar amino acids (e.g. Ser and Thr). They may also include amino acids that are buried in the prefusion conformation, but exposed to solvent in the post-conformation. The replacement amino acids can aliphatic amino acids (Val, lie, Leu and Met), aromatic amino acid (His, Phe, Tyr and Trp), polar amino acids (Thr) with greater size than the replaced amino acids.

In some specific embodiments, a hMPV F protein mutant comprises one or more cavity filling mutations selected from the group consisting of:
(1) substitution of the amino acid at position 49, 291 or 365 with I, V, L, M, F, Y, H;
(2) substitution of the amino acid at position 149 with T, V, or I;
(3) substitution of the amino acid at position 159 with V, I or L;
(4) substitution of the amino acid at position 473 with F or W;

In some specific embodiments, the present disclosure provides a hMPV F mutant comprising one or more cavity filling mutations, wherein the mutant comprises the cavity filling mutations in any of the mutants provided in Tables 12, 15, 16, 22, 25, 26, 27 and 29. hMPV F mutants provided in Tables 12, 15, 16, 22, 25, 26, 27 and 29 are based on same native F0 sequence of hMPV A strain TN/95/3-54 (SEQ ID NO:128) or the consensus F0 sequence of hMPV B strain (SEQ ID NO:129), depending on whether the mutants is a hMPV A or hMPV B F protein mutant. The same introduced mutations in each of the mutants can be made to a native F0 polypeptide sequence of any other hMPV subtype or strain to arrive at different hMPV F mutants, such as a native F0 polypeptide sequence set forth in any of the SEQ ID NOs: 1 to 6 and 99 or from any other hMPV A or B strain. The hMPV F mutants that are based on a native F0 polypeptide sequence of any other hMPV subtype or strain and comprise any of the one or more cavity filling mutations are also within the scope of the invention. In some particular embodiments, a hMPV F protein mutant provided by the present disclosure comprises at least one cavity filling mutation selected from the group consisting of: T49I, S149T or T365I.

### 2-2 (c) Proline Substitution Mutations.

In still other embodiments, the present disclosure provides hMPV F protein mutants that include one or more proline substitution mutations. The term proline substitution mutations” refers to the substitution of an amine acid by a proline to prevent the structural refolding that occurs during transit from the prefusion to post-fusion conformation

In some specific embodiments, the hMPV F protein mutant comprises at least one proline substitution mutations selected from the group consisting of 66P, 110P, 132P, 145P, 187P, 449P and 459P, such as L66P, L110P, S132P, N145P, L187P, V449P and A459P. In some specific embodiments, the present disclosure provides a hMPV F mutant comprising one or more proline substitution mutations, wherein the mutant comprises the proline substitution mutations in any of the mutants provided in Tables 10, 13, 16, 20, 23 and 27-29. hMPV F mutants provided in Tables 10, 13, 16, 20, 23 and 27-29 are based on the same native F0 sequence of hMPV A strain TN/95/3-54 (SEQ ID NO:128) or the consensus F0 sequence of hMPV B strain (SEQ ID NO:129), depending on whether the mutants is a hMPV A or hMPV B F protein mutant. The same introduced mutations in each of the mutants can be made to a native F0 polypeptide sequence of any other hMPV subtype or strain to arrive at different hMPV F mutants, such as a native F0 polypeptide sequence set forth in any of the SEQ ID NOs: 1 to 6 and 99 or from any other hMPV A or B strain. hMPV F mutants that are based on a native F0 polypeptide sequence of any other hMPV subtype or strain and comprise any of the one or more proline substitution mutations are also within the scope of the invention. In some particular embodiments, the hMPV F protein mutant comprises mutation A459P. In some particular embodiments, the hMPV F protein mutant comprises mutation L66P or L187P.

### 2-2 (d) Glycine Replacement Mutations.

In still other embodiments, the present disclosure provides hMPV F protein mutants that include one or more glycine replacement mutation. The term “glycine replacement mutation” refers to the replacement of a glycine by another amino acid in the middle of an α-helix to improve protein stability, preferably an amino acid without Cβ substitution, such as Ala, Leu or Met.
In some specific embodiments, the hMPV F protein mutant comprises at least one glycine replacement mutation selected from the group consisting of G106A, G121A and G239A. In some specific embodiments, the present disclosure provides a hMPV F mutant comprising one or more glycine replacement mutations, wherein the mutant comprises the glycine replacement mutations in any of the mutants provided in Tables 11, 14, 16, 21, 24, 26 and 28. hMPV F mutants provided in Tables 11, 14, 16, 21, 24, 26 and 28 are based on the same native F0 sequence of hMPV A strain TN/95/3-54 (SEQ ID NO:128) or the consensus F0 sequence of hMPV B strain (SEQ ID NO:129), depending on whether the mutants is a hMPV A or hMPV b F protein mutant. The same introduced mutations in each of the mutants can be made to a native F0 polypeptide sequence of any other hMPV subtype or strain to arrive at different hMPV F mutants, such as a native F0 polypeptide sequence set forth in any of the SEQ ID NOs: 1 to 6 and 99 or from any other hMPV A or B strain. hMPV F mutants that are based on a native F0 polypeptide sequence of any other hMPV subtype or strain and comprise any of the one or more glycine replacement mutations are also within the scope of the invention. In some particular embodiments, the hMPV F protein mutant comprises mutation G239A.

### 2-2 (e) Combination of Engineered Disulfide Bond Mutations, Cavity Filling Mutations, Proline Substitution Mutation and Glycine Replacement Mutation.

In another aspect, the present disclosure provides hMPV F protein mutants, which comprise a combination of two or more different types of mutations selected from engineered disulfide bond mutations, cavity filling mutations, proline substitution mutation and glycine replacement mutation each as described above.

In some embodiments, the mutants comprise at least one engineered disulfide bond mutation and at least one cavity filling mutation. In some specific embodiments, the hMPV F mutants include a combination of mutations as noted in Tables 15, 16, 25, 26 and 27.

In some further embodiments, the hMPV F protein mutants comprise at least one engineered disulfide mutation and at least one proline substitution mutation. In some specific embodiments, the hMPV F mutants include a combination of mutations as noted in Tables 13, 16, 23 and 26-28.

In some further embodiments, the hMPV F protein mutants comprise at least one engineered disulfide mutation and at least one glycine replacement mutation. In some specific embodiments, the hMPV F mutants include a combination of mutations as noted in Table 14, 16, 24, 26 and 28.
In some further embodiments, the hMPV F protein mutants comprise at least one proline substitution mutation and at least one cavity filling mutations. In some specific embodiments, the hMPV F mutants include a combination of mutations as noted in Table 29.

In some further embodiments, the hMPV F protein mutants comprise at least one engineered disulfide mutation, at least one cavity filling mutation, and at least one proline substitution mutation. In some specific embodiments, the hMPV F mutants include a combination of mutations as noted in Tables 16, 26 and 27.

In some further embodiments, the hMPV F protein mutants comprise at least one engineered disulfide mutation, at least one at least one proline substitution mutation, and at least one glycine replacement mutation. In some specific embodiments, the hMPV F mutants include a combination of mutations as noted in Tables 16, 27 and 28.

In some further embodiments, the hMPV F protein mutants comprise at least one engineered disulfide mutation, at least one cavity filling mutation, at least one proline substitution mutation, and at least one glycine replacement mutation. In some specific embodiments, the hMPV F mutants include a combination of mutations as noted in Tables 16 and 26.

In some particular embodiments, the hMPV F protein mutant comprises mutation A140C, S149C and L187P.

In some particular embodiments, the hMPV F protein mutant comprises any of the above disclosed mutation or combination of mutations in combination with Q100R and S101R.

In some particular embodiments, the hMPV F protein mutant comprises any of the above disclosed mutation or combination of mutations in combination with any mutation disclosed in WO2022076669, such as for example E26C and G439C; N46C and L158C, T49C and A161C, L50C and V162C, E51C and R163C; E51C and K166C; V104C and N457C, L110C and N322C, A113C and D336C, A116C and A338C, A140C and A147C, S291C and S443C; S293C and S443C; S293C and S444C; S355C and V442C; T365C and V463C, S22C and H435C; G53C and K166C; G53C and V169C; E305C and N457C; S291C and L302C, V47C and A159C; T127C and N153C, G121C and I/F258C, F48C and T160C, and/or T365C and Q455C, L219K, V2311, S376T, G366S, S194Q, K166E, T49E, L187F, L473F, S347Q, H435E, H435D or H435N, G106W, A107F, T160M, L158W, 1128F, A190M, V118F, V118M, Q426W, L165F, V1911, T160V, S149V, 1137L, S1491, V1691, N46V, T49I, V/1122L, S192L, T317L, V162F, V162W, L1051, L105F, L105W, L1341, A117M, S347M, S347K, S347Q, V47M, G261M, 1268M, S470Y, V2311, A374V, 1217V, S355F, A86P, A107P, A113P, T114P, V148P, S443P, D461P, L130P, 1141P, K142P, E146P, L151P, N153P, V162P, A/D185P, D186P, L187P, K188P, N342P, A344P, L66N, L73E, N145E, Q195K, E453Q, L66D, K188R, H368R, D461E, T49E, V262D.

In some other particular embodiments, the present invention provides a hMPV F mutant, wherein the mutant comprises a cysteine (C) at position 140 (140C) and at position 149 (149C), and wherein the mutant comprises a F1 polypeptide and a F2 polypeptide selected from the group consisting of:
(1) a F2 polypeptide comprising the amino acid sequence of SEQ ID NO:30 and a F1 polypeptide comprising the amino acid sequence of SEQ ID NO:29;
(2) a F2 polypeptide comprising an amino acid sequence that is at least 97%, 98% or 99% identical to the amino acid sequence of SEQ ID NO:30 and a F1 polypeptide comprising an amino acid sequence that is at least 97%, 98%, or 99% identical to the amino acid sequence of SEQ ID NO:29.

In some other particular embodiments, the present invention provides a hMPV F mutant, wherein the mutant comprises a cysteine (C) at positions 411, 434, 140 and 149 and wherein the mutant comprises a F1 polypeptide and a F2 polypeptide selected from the group consisting of:
(1) F2 polypeptide comprising the amino acid sequence of SEQ ID NO: 40 and a F1 polypeptide comprising the amino acid sequence of SEQ ID NO:39;
(2) a F2 polypeptide comprising an amino acid sequence that is at least 97%, 98% or 99% identical to the amino acid sequence of SEQ ID NO:40 and a F1 polypeptide comprising an amino acid sequence that is at least 97%, 98%, or 99% identical to the amino acid sequence of SEQ ID NO:39.

In some other particular embodiments, the present invention provides a hMPV F mutant, wherein the mutant comprises a cysteine (C) at positions 411, 434, 140 and 149 and a proline at position 459 and wherein the mutant comprises a F1 polypeptide and a F2 polypeptide selected from the group consisting of:
(1) F2 polypeptide comprising the amino acid sequence of SEQ ID NO: 76 and a F1 polypeptide comprising the amino acid sequence of SEQ ID NO:75;
(2) a F2 polypeptide comprising an amino acid sequence that is at least 97%, 98% or 99% identical to the amino acid sequence of SEQ ID NO:76 and a F1 polypeptide comprising an amino acid sequence that is at least 97%, 98%, or 99% identical to the amino acid sequence of SEQ ID NO:75.

In some other particular embodiments, the present invention provides a hMPV F mutant, wherein the mutant comprises a cysteine (C) at positions 411, 434, 140 and 149 and an alanine at position 239 and wherein the mutant comprises a F1 polypeptide and a F2 polypeptide selected from the group consisting of:
(1) F2 polypeptide comprising the amino acid sequence of SEQ ID NO: 80 and a F1 polypeptide comprising the amino acid sequence of SEQ ID NO:79;
(2) a F2 polypeptide comprising an amino acid sequence that is at least 97%, 98% or 99% identical to the amino acid sequence of SEQ ID NO:80 and a F1 polypeptide comprising an amino acid sequence that is at least 97%, 98%, or 99% identical to the amino acid sequence of SEQ ID NO:79.

In some other particular embodiments, the present invention provides a hMPV F mutant, wherein the mutant comprises a cysteine (C) at positions 411, 434, 140 and 149, a proline at position 459, an alanine at position 239 and an isoleucine at position 49 and 365 and wherein the mutant comprises a F1 polypeptide and a F2 polypeptide selected from the group consisting of:
(1) F2 polypeptide comprising the amino acid sequence of SEQ ID NO: 94 and a F1 polypeptide comprising the amino acid sequence of SEQ ID NO:93;
(2) a F2 polypeptide comprising an amino acid sequence that is at least 97%, 98% or 99% identical to the amino acid sequence of SEQ ID NO:94 and a F1 polypeptide comprising an amino acid sequence that is at least 97%, 98%, or 99% identical to the amino acid sequence of SEQ ID NO:93.

The hMPV F protein mutants provided by the present disclosure can be prepared by routine methods known in the art, such as by expression in a recombinant host system using a suitable vector. Suitable recombinant host cells include, for example, insect cells, mammalian cells, avian cells, bacteria, and yeast cells. Examples of suitable insect cells include, for example, Sf9 cells, Sf21 cells, Tn5 cells, Schneider S2 cells, and High Five cells (a clonal isolate derived from the parental Trichoplusia ni BTI-TN-5B1-4 cell line (Invitrogen)). Examples of suitable mammalian cells include Chinese hamster ovary (CHO) cells, human embryonic kidney cells (HEK293 or Expi293 cells, typically transformed by sheared adenovirus type 5 DNA), NIH-3T3 cells, 293-T cells, Vero cells, and HeLa cells. Suitable avian cells include, for example, chicken embryonic stem cells (e.g., EBx® cells), chicken embryonic fibroblasts, chicken embryonic germ cells, quail fibroblasts (e.g. ELL-O), and duck cells. Suitable insect cell expression systems, such as baculovirus-vectored systems, are known to those of skill in the art and described in, e.g., Summers and Smith, Texas Agricultural Experiment Station Bulletin No. 1555 (1987). Materials and methods for baculovirus/insect cell expression systems are commercially available in kit form from, inter alia, Invitrogen, San Diego Calif. Avian cell expression systems are also known to those of skill in the art and described in, e.g., U.S. Pat. Nos. 5,340,740; 5,656,479; 5,830,510; 6,114,168; and 6,500,668. Similarly, bacterial and mammalian cell expression systems are also known in the art and described in, e.g., Yeast Genetic Engineering (Barr et al., eds., 1989) Butterworths, London.

A number of suitable vectors for expression of recombinant proteins in insect or mammalian cells are well-known and conventional in the art. Suitable vectors can contain a number of components, including, but not limited to one or more of the following: an origin of replication; a selectable marker gene; one or more expression control elements, such as a transcriptional control element (e.g., a promoter, an enhancer, a terminator), and/or one or more translation signals; and a signal sequence or leader sequence for targeting to the secretory pathway in a selected host cell (e.g., of mammalian origin or from a heterologous mammalian or non-mammalian species). For example, for expression in insect cells a suitable baculovirus expression vector, such as pFastBac (Invitrogen), is used to produce recombinant baculovirus particles. The baculovirus particles are amplified and used to infect insect cells to express recombinant protein. For expression in mammalian cells, a vector that will drive expression of the construct in the desired mammalian host cell (e.g., Chinese hamster ovary cells) is used.

The hMPV F protein mutant polypeptides can be purified using any suitable methods. For example, methods for purifying hMPV F protein mutant polypeptides by immunoaffinity chromatography are known in the art. Ruiz-Arguello et al., J. Gen. Virol., 85:3677-3687 (2004). Suitable methods for purifying desired proteins including precipitation and various types of chromatography, such as hydrophobic interaction, ion exchange, affinity, chelating, and size exclusion are well-known in the art. Suitable purification schemes can be created using two or more of these or other suitable methods. If desired, the hMPV F protein mutant polypeptides can include a “tag” that facilitates purification, such as an epitope tag, a strep II tag or a histidine (HIS) tag. Such tagged polypeptides can conveniently be purified, for example from conditioned media, by chelating chromatography or affinity chromatography. Below table 1 provides representative sequences from hMPV A and B F0 polypeptide

TABLE 1
F protein sequences from selected hMPV strains.

| Strain | SEQ | |
|---|---|---|
| (subtype)/ | ID | |
| GenBank_aa | NO | F0 protein sequence |
| TN/95/3-54 | 1 | MSWKVVIIFSLLITPQHGLKESYLEESCSTITEGYLSVLRTGWY |
| (A2b)/ | | TNVFTLEVGDVENLTCADGPSLIKTELDLTKSALRELKTVSAD |
| ACJ53569.1 | | QLAREEQIENPRQSRFVLGAIALGVATAAAVTAGVAIAKTIRLE |
| | | SEVTAIKNALKKTNEAVSTLGNGVRVLATAVRELKDFVSKNLT |
| | | RAINKNKCDIDDLKMAVSFSQFNRRFLNVVRQFSDNAGITPAI |
| | | SLDLMTDAELARAVSNMPTSAGQIKLMLENRAMVRRKGFGIL |
| | | IGVYGSSVIYMVQLPIFGVIDTPCWIVKAAPSCSEKKGNYACL |
| | | LREDQGWYCQNAGSTVYYPNEKDCETRGDHVFCDTAAGIN |
| | | VAEQSKECNINISTTNYPCKVSTGRHPISMVALSPLGALVACY |
| | | KGVSCSIGSNRVGIIKQLNKGCSYITNQDADTVTIDNTVYQLS |
| | | KVEGEQHVIKGRPVSSSFDPVKFPEDQFNVALDQVFENIENS |
| | | QALVDQSNRILSSAEKGNTGFIIVIILIAVLGSSMILVSIFIIIKKTK |
| | | KPTGAPPELSGVTNNGFIPHS |
| CAN00- | 2 | MSWKVVIIFSLLITPQHGLKESYLEESCSTITEGYLSVLRTGWY |
| 14 (A1)/ | | TNVFTLEVGDVENLTCADGPSLIKTELDLTKSALRELRTVSAD |
| AAN52913.1 | | QLAREEQIENPRQSRFVLGAIALGVATAAAVTAGVAIAKTIRLE |
| | | SEVTAIKNALKKTNEAVSTLGNGVRVLATAVRELKDFVSKNLT |
| | | RAINKNKCDIADLKMAVSFSQFNRRFLNVVRQFSDNAGITPAI |
| | | SLDLMTDAELARAVSNMPTSAGQIKLMLENRAMVRRKGFGIL |
| | | IGVYGSSVIYMVQLPIFGVIDTPCWIVKAAPSCSEKKGNYACL |
| | | LREDQGWYCQNAGSTVYYPNEKDCETRGDHVFCDTAAGIN |
| | | VAEQSKECNINISTTNYPCKVSTGRHPISMVALSPLGALVACY |
| | | KGVSCSIGSNRVGIIKQLNKGCSYITNQDADTVTIDNTVYQLS |
| | | KVEGEQHVIKGRPVSSSFDPVKFPEDQFNVALDQVFESIENS |
| | | QALVDQSNRILSSAEKGNTGFIIVIILIAVLGSTMILVSVFIIIKKT |
| | | KKPTGAPPELSGVTNNGFIPHN |
| TN/00/3-1 | 3 | MSWKWVIIFSLLITPQHGLKESYLEESCSTITEGYLSVLRTGWY |
| (A2a)/ | | TNVFTLEVGDVENLTCSDGPSLIKTELDLTKSALRELKTVSAD |
| ACJ53563.1 | | QLAREEQIENPRQSRFVLGAIALGVATAAAVTAGVAIAKTIRLE |
| | | SEVTAIKNALKTTNEAVSTLGNGVRVLATAVRELKDFVSKNLT |
| | | RAINKNKCDIDDLKMAVSFSQFNRRFLNVVRQFSDNAGITPAI |
| | | SLDLMTDAELARAVSNMPTSAGQIKLMLENRAMVRRKGFGIL |
| | | IGVYGSSVIYMVQLPIFGVIDTPCWIVKAAPSCSEKKGNYACL |
| | | LREDQGWYCQNAGSTVYYPNEKDCETRGDHVFCDTAAGIN |
| | | VAEQSKECNINISTTNYPCKVSTGRHPISMVALSPLGALVACY |
| | | KGVSCSIGSNRVGIIKQLNKGCSYITNQDADTVTIDNTVYQLS |
| | | KVEGEQHVIKGRPVSSSFDPIKFPEDQFNVALDQVFENIENS |
| | | QALVDQSNRILSSAEKGNTGFIIVIILIAVLGSSMILVSIFIIIKKTK |
| | | KPTGAPPELSGVTNNGFIPHS |
| Consensus | 4 | MSWKVMIIISLLITPQHGLKESYLEESCSTITEGYLSVLRTGWY |
| hMPV B | | TNVFTLEVGDVENLTCTDGPSLIKTELDLTKSALRELKTVSAD |
| | | QLAREEQIENPRQSRFVLGAIALGVATAAAVTAGIAIAKTIRLE |
| | | SEVNAIKGALKTTNEAVSTLGNGVRVLATAVRELKEFVSKNLT |
| | | SAINKNKCDIADLKMAVSFSQFNRRFLNVVRQFSDNAGITPAI |
| | | SLDLMNDAELARAVSYMPTSAGQIKLMLENRAMVRRKGFGIL |
| | | IGVYGSSVIYMVQLPIFGVINTPCWIIKAAPSCSEKDGNYACLL |
| | | REDQGWYCKNAGSTVYYPNEKDCETRGDHVFCDTAAGINV |
| | | AEQSRECNINISTTNYPCKVSTGRHPISMVALSPLGALVACYK |
| | | GVSCSIGSNQVGIIKQLPKGCSYITNQDADTVTIDNTVYQLSK |
| | | VEGEQHVIKGRPVSSSFDPIRFPEDQFNVALDQVFESIENSQ |
| | | ALVDQSNKILNSAEKGNTGFIIVIILIAVLGLTMISVSIIIIIKKTRKP |
| | | TGAPPELNGVTNGGFIPHS |
| JPS03-194 | 5 | MSWKVMIIISLLITPQHGLKESYLEESCSTITEGYLSVLRTGWY |
| (B1)/ | | TNVFTLEVGDVENLTCTDGPSLIKTELDLTKSALRELKTVSAD |
| AAS22117.1 | | QLAREEQIENPRQSRFVLGAIALGVATAAAVTAGIAIAKTIRLE |
| | | SEVNAIKGALKQTNEAVSTLGNGVRVLATAVRELKEFVSKNL |
| | | TSAINRNKCDIADLKMAVSFSQFNRRFLNVVRQFSDNAGITP |
| | | AISLDLMTDAELARAVSYMPTSAGQIKLMLENRAMVRRKGFG |
| | | ILIGVYGSSVIYMVQLPIFGVIDTPCWIIKAAPSCSEKNGNYAC |
| | | LLREDQGWYCKNAGSTVYYPNEKDCETRGDHVFCDTAAGIN |
| | | VAEQSRECNINISTTNYPCKVSTGRHPISMVALSPLGALVACY |
| | | KGVSCSIGSNRVGIIKQLPKGCSYITNQDADTVTIDNTVYQLS |
| | | KVEGEQHVIKGRPVSSSFDPIRFPEDQFNVALDQVFESIENS |
| | | QALVEQSNKILNSAEKGNTGFIIVIILVAVLGLTMISVSIIIIIKKTR |
| | | KPTGAPPELNGVTNGGFIPHS |
| HR18786-11 | 6 | MSWKVMIIISLLITPQHGLKESYLEESCSTITEGYLSVLRTGWY |
| (B2)/ | | TNVFTLEVGDVENLTCTDGPSLIKTELDLTKSALRELKTVSAD |
| ANW37992.1 | | QLAREEQIENPRQSRFVLGAIALGVATAAAVTAGIAIAKTIRLE |
| | | SEVNAIKGALKTTNEAVSTLGNGVRVLATAVRELKEFVSKNLT |
| | | SAINKNKCDIADLKMAVSFSQFNRRFLNVVRQFSDNAGITPAI |
| | | SLDLMNDAELARAVSYMPTSAGQIKLMLENRAMVRRKGFGIL |
| | | IGVYGSSVIYMVQLPIFGVINTPCWIIKAAPSCSEKDGNYACLL |
| | | REDQGWYCKNAGSTVYYPNEKDCETRGDHVFCDTAAGINV |
| | | AEQSRECNINISTTNYPCKVSTGRHPISMVALSPLGALVACYK |
| | | GVSCSTGSNQVGIIKQLPKGCSYITNQDADTVTIDNTVYQLSK |
| | | VEGEQHVIKGRPVSNSFDPIRFPEDQFNVALDQVFESIENSQ |
| | | ALVDQSNKILNSAEKGNTGFIIVIILIAVLGLTMISVSIIIIIKKTRKP |
| | | AGAPPELNGVTNGGFIPHS |
| 6073-B2 (B2)/ | 99 | MSWKVMIIISLLITPQHGLKESYLEESCSTITEGYLSVLRTGWY |
| QDA18370.1 | | TNVFTLEVGDVENLTCTDGPSLIKTELDLTKSALRELKTVSAD |
| | | QLAREEQIENPRQSRFVLGAIALGVATAAAVTAGIAIAKTIRLE |
| | | SEVNAIKGALKTTNEAVSTLGNGVRVLATAVRELKEFVSKNLT |
| | | SAINKNKCDIADLKMAVSFSQFNRRFLNVVRQFSDNAGITPAI |
| | | SLDLMNDAELARAVSYMPTSAGQIKLMLENRAMVRRKGFGIL |
| | | IGVYGSSVIYMVQLPIFGVINTPCWIIKAAPSCSEKDGNYACLL |
| | | REDQGWYCKNAGSTVYYPNEKDCETRGDHVFCDTAAGINV |
| | | AEQSRECNINISTTNYPCKVSTGRHPISMVALSPLGALVACYK |
| | | GVSCSIGSNQVGIIKQLPKGCSYITNQDADTVTIDNTVYQLSK |
| | | VEGEQHVIKGRPVSNSFDPIRFPEDQFNVALDQVFESIENSQ |
| | | ALVDQSNKILNSAEKGNTGFIIVIILIAVLGLTMISVSIIIIIKKTRKP |
| | | AGAPPELNGVTNGGFIPHS |

Table 2 provides the amino acid sequence of F1 polypeptide without transmembrane and intracellular domains and F2 polypeptide of variants of mutant hMPV083 (based on F protein sequence from TN/95/3-54 strain) to illustrate how a particular set of mutations applies to any hMPV A wild type F protein.

TABLE 2
Variants of Mutant hMPV083 and comprising introduced mutations T411C-Q434C,
A140C-S149C, A459P, G239A, T49I, T365I

| | | | Amino Acid Sequence: |
|---|---|---|---|
| | Poly- | SEQ | (residues 103-489 for F1 polypeptide and |
| Mutant | peptide | ID | residues 19-102 for F2 polypeptide) |
| hMPV083- | F1 | 11 | FVLGAIALGVATAAAVTAGVAIAKTIRLESEVTAIKNCLKKT |
| TN/95/3-54 | | | NEAVCTLGNGVRVLATAVRELKDFVSKNLTRAINKNKCDI |
| (A2b)/ | | | DDLKMAVSFSQFNRRFLNVVRQFSDNAGITPAISLDLMT |
| ACJ53569.1 | | | DAELARAVSNMPTSAAQIKLMLENRAMVRRKGFGILIGV |
| | | | YGSSVIYMVQLPIFGVIDTPCWIVKAAPSCSEKKGNYACL |
| | | | LREDQGWYCQNAGSTVYYPNEKDCETRGDHVFCDTAA |
| | | | GINVAEQSKECNINISTTNYPCKVSIGRHPISMVALSPLGA |
| | | | LVACYKGVSCSIGSNRVGIIKQLNKGCSYICNQDADTVTI |
| | | | DNTVYQLSKVEGECHVIKGRPVSSSFDPVKFPEDQFNVP |
| | | | LDQVFENIENSQALVDQSNRILSSAEKGNT |
| | F2 | 12 | LKESYLEESCSTITEGYLSVLRTGWYTNVFILEVGDVENL |
| | | | TCADGPSLIKTELDLTKSALRELKTVSADQLAREEQIENP |
| | | | RQSR |
| 083- | F1 | 13 | FVLGAIALGVATAAAVTAGVAIAKTIRLESEVTAIKNCLKKT |
| CAN00- | | | NEAVCTLGNGVRVLATAVRELKDFVSKNLTRAINKNKCDI |
| 14 (A1)/ | | | ADLKMAVSFSQFNRRFLNVVRQFSDNAGITPAISLDLMT |
| AAN52913.1 | | | DAELARAVSNMPTSAAQIKLMLENRAMVRRKGFGILIGV |
| | | | YGSSVIYMVQLPIFGVIDTPCWIVKAAPSCSEKKGNYACL |
| | | | LREDQGWYCQNAGSTVYYPNEKDCETRGDHVFCDTAA |
| | | | GINVAEQSKECNINISTTNYPCKVSIGRHPISMVALSPLGA |
| | | | LVACYKGVSCSIGSNRVGIIKQLNKGCSYICNQDADTVTI |
| | | | DNTVYQLSKVEGECHVIKGRPVSSSFDPVKFPEDQFNVP |
| | | | LDQVFESIENSQALVDQSNRILSSAEKGNT |
| | F2 | 14 | LKESYLEESCSTITEGYLSVLRTGWYTNVFILEVGDVENL |
| | | | TCADGPSLIKTELDLTKSALRELRTVSADQLAREEQIENP |
| | | | RQSR |
| 083- | F1 | 15 | FVLGAIALGVATAAAVTAGVAIAKTIRLESEVTAIKNCLKTT |
| TN/00/3-1 | | | NEAVCTLGNGVRVLATAVRELKDFVSKNLTRAINKNKCDI |
| (A2a)/ | | | DDLKMAVSFSQFNRRFLNVVRQFSDNAGITPAISLDLMT |
| ACJ53563.1 | | | DAELARAVSNMPTSAAQIKLMLENRAMVRRKGFGILIGV |
| | | | YGSSVIYMVQLPIFGVIDTPCWIVKAAPSCSEKKGNYACL |
| | | | LREDQGWYCQNAGSTVYYPNEKDCETRGDHVFCDTAA |
| | | | GINVAEQSKECNINISTTNYPCKVSIGRHPISMVALSPLGA |
| | | | LVACYKGVSCSIGSNRVGIIKQLNKGCSYICNQDADTVTI |
| | | | DNTVYQLSKVEGECHVIKGRPVSSSFDPIKFPEDQFNVP |
| | | | LDQVFENIENSQALVDQSNRILSSAEKGNT |
| | F2 | 16 | LKESYLEESCSTITEGYLSVLRTGWYTNVFILEVGDVENL |
| | | | TCSDGPSLIKTELDLTKSALRELKTVSADQLAREEQIENP |
| | | | RQSR |

Table 3 provides the amino acid sequence of F0 polypeptide without transmembrane and intracellular domains and F2 polypeptide of variants of mutant hMPV83 (based on hMPV B consensus sequence) to illustrate how a particular set of mutations applies to any hMPV B wild type F protein.

TABLE 3
Variants of Mutant hMPV083 and comprising introduced mutations T411C-Q434C,
A140C-S149C, A459P, G239A, T491, T365I

| | | | Amino Acid Sequence: |
|---|---|---|---|
| | Poly- | SEQ | (residues 103-489 for F1 polypeptide and |
| Mutant ID | peptide | ID | residues 19-102 for F2 polypeptide) |
| 083- | F1 | 17 | FVLGAIALGVATAAAVTAGIAIAKTIRLESEVNAIKGCLKTT |
| Consensus | | | NEAVCTLGNGVRVLATAVRELKEFVSKNLTSAINKNKCDI |
| hMPV B | | | ADLKMAVSFSQFNRRFLNVVRQFSDNAGITPAISLDLMN |
| | | | DAELARAVSYMPTSAAQIKLMLENRAMVRRKGFGILIGV |
| | | | YGSSVIYMVQLPIFGVINTPCWIIKAAPSCSEKDGNYACLL |
| | | | REDQGWYCKNAGSTVYYPNEKDCETRGDHVFCDTAAG |
| | | | INVAEQSRECNINISTTNYPCKVSIGRHPISMVALSPLGAL |
| | | | VACYKGVSCSIGSNQVGIIKQLPKGCSYICNQDADTVTID |
| | | | NTVYQLSKVEGECHVIKGRPVSSSFDPIRFPEDQFNVPL |
| | | | DQVFESIENSQALVDQSNKILNSAEKGNT |
| | F2 | 18 | LKESYLEESCSTITEGYLSVLRTGWYTNVFILEVGDVENL |
| | | | TCTDGPSLIKTELDLTKSALRELKTVSADQLAREEQIENP |
| | | | RQSR |
| 083- | F1 | 19 | FVLGAIALGVATAAAVTAGIAIAKTIRLESEVNAIKGCLKQT |
| JPS03-194 | | | NEAVCTLGNGVRVLATAVRELKEFVSKNLTSAINRNKCDI |
| (B1)/ | | | ADLKMAVSFSQFNRRFLNVVRQFSDNAGITPAISLDLMT |
| AAS22117.1 | | | DAELARAVSYMPTSAAQIKLMLENRAMVRRKGFGILIGV |
| | | | YGSSVIYMVQLPIFGVIDTPCWIIKAAPSCSEKNGNYACLL |
| | | | REDQGWYCKNAGSTVYYPNEKDCETRGDHVFCDTAAG |
| | | | INVAEQSRECNINISTTNYPCKVSIGRHPISMVALSPLGAL |
| | | | VACYKGVSCSIGSNRVGIIKQLPKGCSYICNQDADTVTID |
| | | | NTVYQLSKVEGECHVIKGRPVSSSFDPIRFPEDQFNVPL |
| | | | DQVFESIENSQALVEQSNKILNSAEKGNT |
| | F2 | 20 | LKESYLEESCSTITEGYLSVLRTGWYTNVFILEVGDVENL |
| | | | TCTDGPSLIKTELDLTKSALRELKTVSADQLAREEQIENP |
| | | | RQSR |
| 083- | F1 | 21 | FVLGAIALGVATAAAVTAGIAIAKTIRLESEVNAIKGCLKTT |
| HR18786-11 | | | NEAVCTLGNGVRVLATAVRELKEFVSKNLTSAINKNKCDI |
| (B2)/ | | | ADLKMAVSFSQFNRRFLNVVRQFSDNAGITPAISLDLMN |
| ANW37992.1 | | | DAELARAVSYMPTSAAQIKLMLENRAMVRRKGFGILIGV |
| | | | YGSSVIYMVQLPIFGVINTPCWIIKAAPSCSEKDGNYACLL |
| | | | REDQGWYCKNAGSTVYYPNEKDCETRGDHVFCDTAAG |
| | | | INVAEQSRECNINISTTNYPCKVSIGRHPISMVALSPLGAL |
| | | | VACYKGVSCSTGSNQVGIIKQLPKGCSYICNQDADTVTID |
| | | | NTVYQLSKVEGECHVIKGRPVSNSFDPIRFPEDQFNVPL |
| | | | DQVFESIENSQALVDQSNKILNSAEKGNT |
| | F2 | 22 | LKESYLEESCSTITEGYLSVLRTGWYTNVFILEVGDVENL |
| | | | TCTDGPSLIKTELDLTKSALRELKTVSADQLAREEQIENP |
| | | | RQSR |
| 083- | F1 | 100 | FVLGAIALGVATAAAVTAGIAIAKTIRLESEVNAIKGCLKTT |
| 6073-B2 | | | NEAVCTLGNGVRVLATAVRELKEFVSKNLTSAINKNKCDI |
| (B2)/ | | | ADLKMAVSFSQFNRRFLNVVRQFSDNAGITPAISLDLMN |
| QDA18370.1 | | | DAELARAVSYMPTSAAQIKLMLENRAMVRRKGFGILIGV |
| | | | YGSSVIYMVQLPIFGVINTPCWIIKAAPSCSEKDGNYACLL |
| | | | REDQGWYCKNAGSTVYYPNEKDCETRGDHVFCDTAAG |
| | | | INVAEQSRECNINISTTNYPCKVSIGRHPISMVALSPLGAL |
| | | | VACYKGVSCSIGSNQVGIIKQLPKGCSYICNQDADTVTID |
| | | | NTVYQLSKVEGECHVIKGRPVSNSFDPIRFPEDQFNVPL |
| | | | DQVFESIENSQALVDQSNKILNSAEKGNT |
| | F2 | 101 | LKESYLEESCSTITEGYLSVLRTGWYTNVFILEVGDVENL |
| | | | TCTDGPSLIKTELDLTKSALRELKTVSADQLAREEQIENP |
| | | | RQSR |

### 3. Nucleic Acids Encoding hMPV F Protein Mutants

In another aspect, the present invention provides nucleic acid molecules that encode a hMPV F protein mutant described herein above. These nucleic acid molecules include DNA, cDNA, and RNA sequences. Nucleic acid molecules that encode only a F2 polypeptide or only a F1 polypeptide of a hMPV F mutant are also encompassed by the invention. The nucleic acid molecule can be incorporated into a vector, such as an expression vector.

In some embodiments, the nucleic acid molecule encodes a precursor F0 polypeptide that, when expressed in an appropriate cell, is processed into a disclosed hMPV F mutant. In some embodiments, the nucleic acid molecule encodes a precursor F0 polypeptide that, when expressed in an appropriate cell, is processed into a disclosed hMPV F mutant, wherein the precursor F0 polypeptide includes, from N- to C-terminus, a signal peptide, a F2 polypeptide, and a F1 polypeptide. In some embodiments, the signal peptide comprises the amino acid sequence set forth as positions 1-18 of any one SEQ ID NOs: 1 to 6 and 99, wherein the amino acid positions correspond to the amino acid sequence of a reference of SEQ ID NO:1.

In a preferred embodiment, the nucleic acid is an RNA, more preferably an mRNA. In a preferred embodiment, the mRNA encodes a precursor F0 polypeptide that, when expressed in an appropriate cell, is processed into a full length hMPV F protein mutant disclosed herein (i.e. comprising one or more mutations, a full length F1 polypeptide and a full length F2 polypeptide). A full-length F1 polypeptide of the hMPV F mutants corresponds to amino acid positions 103-539 of the native hMPV F0 precursor, and includes (from N- to C-terminus) an extracellular region (residues 103 to 489), a transmembrane domain (residues 490-514), and a cytoplasmic domain (residues 515-539). In a preferred embodiment, the nucleic acid is an mRNA comprising a chemically modified nucleotide. In a preferred embodiment, the nucleic acid is an mRNA comprising a chemically modified nucleotide, preferably 1-methylpseudouridine. Preferably, all the uridines of the RNA are replaced by 1-methylpseudouridine.

In some embodiments, the nucleic acid molecule encodes a mutant selected from the group consisting of:
(1) a mutant comprising at least one engineered disulfide bond mutation;
(2) a mutant comprising at least one cavity filling mutation;
(3) a mutant comprising at least one proline substitution mutation;
(4) a mutant comprising at least one glycine replacement mutation;
(5) a mutant comprising a combination of at least one engineered disulfide mutation and at least one cavity filling mutation;
(6) a mutant comprising a combination of at least one engineered disulfide mutation and at least one proline substitution mutation;
(7) a mutant comprising a combination of at least one engineered disulfide mutation and a least one glycine replacement mutation;
(8) a mutant comprising a combination of at least one engineered disulfide mutation, at least one cavity filling mutation and at least one proline substitution mutation;
(9) a mutant comprising a combination of at least one engineered disulfide mutation, at least one cavity filling mutation, and a least one glycine replacement mutation;
(10) a mutant comprising a combination of at least one engineered disulfide mutation, at least one proline substitution mutation and a least one glycine replacement mutation; and,
(11) a mutant comprising a combination of at least one engineered disulfide mutation, at least one cavity filling mutation, at least one proline substitution mutation and a least one glycine replacement mutation.

In some specific embodiments, the present disclosure provides a nucleic acid molecule which encodes a mutant selected from the group consisting of:
(1) a mutant comprising a combination of substitutions 140C and 149C;
(2) a mutant comprising a combination of substitutions 140C, 149C, 411C and 434C;
(3) a mutant comprising a combination of substitutions 140C, 149C, 411C, 434C and 459P;
(4) a mutant comprising a combination of substitutions 140C, 149C, 411C, 434C and 365I;
(5) a mutant comprising a combination of substitutions 140C, 149C, 411C, 434C and G239A;
(6) a mutant comprising a combination of substitutions 140C, 149C, 411C, 434C, 459P, G239A, 49I and 365I;
(7) a mutant comprising a combination of substitutions 411C, 434C, 141C and 161C;
(8) a mutant comprising a combination of substitutions 411C, 434C, 141C, 161C and 459P;
(9) a mutant comprising a combination of substitutions 411C, 434C, 141C, 161C and 49I;
(10) a mutant comprising a combination of substitutions 411C, 434C, 141C, 161C and 365I;
(10) a mutant comprising a combination of substitutions 411C, 434C, 141C, 161C and G239A;
(10) a mutant comprising a combination of substitutions 411C, 434C, 141C, 161C and 149T;
(10) a mutant comprising a combination of substitutions 411C, 434C, 141C, 161C, 459P, G239A, 49I, 149T and 365I; and
(10) a mutant comprising a combination of substitutions 411C, 434C, 141C, 161C and 365I; and
(11) a mutant comprising a combination of substitutions 411C, 434C, 146C, 160C, 459P, G239A, 49I, 149T and 365I.

In some specific embodiments, the present disclosure provides a nucleic acid molecule, preferably a mRNA, more preferably a mRNA wherein all the uridines are replaced by 1-methylpseudouridine, said nucleic acid encoding a precursor F0 polypeptide that, when expressed in an appropriate cell, is processed into a full length hMPV F protein mutant disclosed herein comprising the mutations selected from the group of (1) A140C and S149C,
(2) A140C, S149C, T411C and Q434C,
(3) A140C, S149C, T411C, Q434C and A459P,
(4) A140C, S149C, T411C, Q434C and T365I,
(5) A140C, S149C, T411C, Q434C and G239A,
(6) A140C, S149C, T411C, Q434C, A459P, G239A, T49I and T365I,
(7) T411C, Q434C, L141C and A161C,
(8) T411C, Q434C, L141C, A161C and A459P,
(9) T411C, Q434C, L141C, A161C and T49I,
(10) T411C, Q434C, L141C, A161C and T365I,
(11) T411C, Q434C, L141C, A161C and G239A,
(12) T411C, Q434C, L141C, A161C and S149T,
(13) T411C, Q434C, L141C, A161C, A459P, G239A, T49I, S149T and T365I, and,
(14) T411C, Q434C, E146C, T160C, A459P, G239A, T49I, S149T and T365I.

In some specific embodiments, the present disclosure provides a nucleic acid molecule, preferably a mRNA, more preferably a mRNA wherein all the uridines are replaced by 1-methylpseudouridine, said nucleic acid encoding a precursor F0 polypeptide that, when expressed in an appropriate cell, is processed into a full length hMPV F protein mutant disclosed comprising the mutations selected from the group consisting of
(1) T150C, R156C and A459P;
(2) T150C, R156C and T49I;
(3) T150C, R156C, T49I and A459P;
(4) A140C, S149C, T411C, and Q434C;
(5) L141C, A161C, T411C, and 434C;
(6) A140C, S149C, T411C, Q434C, and A459P;
(7) A140C, S149C, G239A, T411C and Q434C;
(8) L141C, A161C, T411C, Q434C and A459P;
(9) L141C, A161C, G239A, T411C and Q434C;
(10) T49I, T150C, R156C, G239A and A459P;
(11) A140C, S149C, G239A, T411C, Q434C and A459P;
(12)T49I, A140C, S149C, G239A, T411C, Q434C and A459P;
(13)T49I, A140C, S149C, G239A, T365I, T411C, Q434C and A459P;
(14) L141C, A161C, G239A, T411C, Q434C and A459P;
(15) T49I, L141C, A161C, G239A, T411C, Q434C and A459P;
(16) L141C, A161C, S149T, G239A, T411C, Q434C and A459P;
(17) T49I, L141C, A161C, S149T, G239A, T411C, Q434C and A459P;
(18) T49I, L141C, A161C, S149T, G239A, T365I, T411C, Q434C and A459P;
(19) T49I, S149T and A459P;
(20) A140C, S149C and A459P;
(21) T49I, A140C and S149C;
(22) T49I, A140C, S149C and A459P;
(23) T49I, L141C, A161C, T411C and Q434C;
(24) T49I, L141C, A161C, T411C, Q434C and A459P;
(25) L141C, A161C and S149T;
(26) L141C, A161C, S149T and A459P;
(27) T49I, L141C, A161C and S149T, and,
(28) T49I, L141C, A161C, S149T and A459P.

In some specific embodiments, the present disclosure provides a nucleic acid molecule, preferably a mRNA, more preferably a mRNA wherein all the uridines are replaced by 1-methylpseudouridine, said nucleic acid encoding a precursor F0 polypeptide that, when expressed in an appropriate cell, is processed into a full length hMPV F protein mutant disclosed herein comprising the mutations selected from the group consisting of
(1) L66P;
(2) L187P;
(4) A140C, S149C and L187P;
(5) T49I;
(6)T365I; and,
(7) T49I and T365I.

In some specific embodiments, the present disclosure provides a nucleic acid molecule, preferably a mRNA, more preferably a mRNA wherein all the uridines are replaced by 1-methylpseudouridine, said nucleic acid encoding a precursor F0 polypeptide that, when expressed in an appropriate cell, is processed into a full length hMPV F protein mutant disclosed herein comprising the mutations selected from the group consisting of
(1) L187P, Q100R and S101R; and,
(2) A140C, S149C, L187P, Q100R and S101R.

### C. PIV1 Mutants

The present disclosure relates to PIV1 F protein mutants, immunogenic compositions comprising the PIV1 F protein mutants, methods for producing the PIV1 F protein mutants, compositions comprising the PIV1 F protein mutants, and nucleic acids that encode the PIV1 F protein mutants.

### 1. EXEMPLARY EMBODIMENTS (E) OF THE INVENTION

E1. A mutant of a wild-type PIV1 F protein, which mutant comprises a F1 polypeptide and a F2 polypeptide, wherein the mutant comprises at least one amino acid mutation relative to the amino acid sequence of the wild-type PIV1 F protein, and wherein the amino acid mutation is selected from the group consisting of:
(1) at least one engineered disulfide bond mutation;
(2) at least one cavity filling mutation;
(3) at least one proline substitution mutation;
(4) at least one glycine replacement mutation;
(5) a cleavage site mutation;
(6) a combination of at least one engineered disulfide mutation and at least one cavity filling mutation;
(7) a combination of at least one engineered disulfide mutation and at least one proline substitution mutation;
(8) a combination of at least one engineered disulfide mutation and a least one glycine replacement mutation;
(8) a combination of at least one engineered disulfide mutation, at least one cavity filling mutation and at least one proline substitution mutation;
(10) a combination of at least one engineered disulfide mutation, at least one cavity filling mutation, and a least one glycine replacement mutation;
(11) a combination of at least one engineered disulfide mutation, at least one proline substitution mutation and a least one glycine replacement mutation;
(12) a combination of at least one engineered disulfide mutation, at least one cavity filling mutation, at least one proline substitution mutation and a least one glycine replacement mutation
(13) a combination of a cleavage site mutation and at least one engineered disulfide mutation;
(14) a combination of a cleavage site mutation and at least one cavity filling mutation;
(15) a combination of a cleavage site mutation and at least one proline substitution mutation;
(16) a combination of a cleavage site mutation and at least one glycine replacement mutation;
(17) a combination of a cleavage site mutation and at least one engineered disulfide mutation and at least one cavity filling mutation;
(18) a combination of a cleavage site mutation and at least one engineered disulfide mutation and at least one proline substitution mutation;
(19) a combination of a cleavage site mutation and at least one engineered disulfide mutation and a least one glycine replacement mutation;
(20) a combination of a cleavage site mutation, at least one engineered disulfide mutation, at least one cavity filling mutation and at least one proline substitution mutation;
(21) a combination of a cleavage site mutation, at least one engineered disulfide mutation, at least one cavity filling mutation, and a least one glycine replacement mutation;
(22) a combination of a cleavage site mutation, at least one engineered disulfide mutation, at least one proline substitution mutation and a least one glycine replacement mutation;
(23) a combination of a cleavage site mutation, at least one engineered disulfide mutation, at least one cavity filling mutation, at least one proline substitution mutation and a least one glycine replacement mutation;
(24) a combination of a cleavage site mutation, at least one cavity filling mutation and at least one proline substitution mutation;
(25) a combination of a cleavage site mutation, at least one cavity filling mutation and a least one glycine replacement mutation;
(26) a combination of a cleavage site mutation, at least one proline substitution mutation and at least one glycine replacement mutation;
(27) a combination of at least one cavity filling mutation and at least one proline substitution mutation;
(28) a combination of at least one cavity filling mutation and a least one glycine replacement mutation (29) a combination of at least one proline substitution mutation and a least one glycine replacement mutation:
(30) a combination of at least one cavity filling mutation, at least one proline substitution mutation and a least one glycine replacement mutation.
E2. The mutant according to E1 wherein the mutant comprises an engineered disulfide mutation.
E3. The mutant according to E1 or E2 wherein the engineered disulfide mutation is Q92C-G134C.
E4. The mutant according to any one of E1 to E3, wherein the mutant comprises a cavity filling mutation.
E5. The mutant according to any one of E1 to E4, wherein the cavity filling mutation is selected from T198A, Q92A, Q92L, A466L, A466V, A4661, S473V, S473L, S4731, S473A, A480L and A480V.
E6. The mutant according to E5, wherein the cavity filling mutation is T198A.
E7. The mutant according to E5, wherein the cavity filling mutation is Q92A.
E8. The mutant according to E5, wherein the cavity filling mutation is Q92L.
E9. The mutant according to E5, wherein the cavity filling mutation is A466L.
E10. The mutant according to E5, wherein the cavity filling mutation is A466V.
E11. The mutant according to E5, wherein the cavity filling mutation is S473V.
E12. The mutant according to E5, wherein the cavity filling mutation is S473L.
E13. The mutant according to E5, wherein the cavity filling mutation is S4731.
E14. The mutant according to E5, wherein the cavity filling mutation is S473A.
E15. The mutant according to E5, wherein the cavity filling mutation is A480L.
E16. The mutant according to E5, wherein the cavity filling mutation is A480V.
E17. The mutant according to any one of E1 to E4, wherein the mutant comprises two or three cavity filling mutations selected from T198A, Q92A, Q92L, A466L, A466V, A4661, S473V, S473L, S4731, S473A, A480L and A480V.
E18. The mutant according to E17, wherein the cavity filling mutations are A466L and S473L.
E19. The mutant according to E17, wherein the cavity filling mutations are A4661 and S4731.
E20. The mutant according to any one of E18 or E19 further comprising the cavity filling mutation A480L or A480V.
E21. The mutant according to any one of E1 to E20, wherein the mutant comprises a proline substitution mutation.
E22. The mutant according to E21, wherein the proline substitution mutation is A128P.
E23. The mutant according to any one of E1 to E22, wherein the mutant comprises a glycine replacement mutation.
E24. The mutant according to E23, wherein the glycine replacement mutation is G134 Aor G134L.
E25. The mutant according to E24, wherein the glycine replacement mutation is G134A.
E26. The mutant according to any one of E1 to E25, wherein the mutant comprises a cleavage site mutation.
E27. The mutant according to E26, wherein the cleavage site mutation is F113G and F114S.
E28. The mutant according to E1, wherein the mutant comprises the mutations selected from from the group consisting of:
(1) Q92C-G134C;
(2) A466L;
(3) A466V;
(4) S473V;
(5) S473L;
(6) A480L;
(7) A466L and S473A;
(8) A466L and S473L;
(9) T198A;
(10) G134A;
(11) A128P;
(12) F113G, F114S, Q92C-G134C, A466L, S473L and A480L;
(13) Q92C-G134C, A466L, S473L and A480L;
(14) Q92C-G134C, A466L and S473L;
(15) F113G, F114S, Q92C-G134C, A466V, S473V and A480V;
(16) Q92C-G134C, A466V, S473V and A480V;
(17) Q92C-G134C, A466V and S473V;
(18) F113G, F114S, A466L, S473L, A480L and G134A;
(19) A466L, S473L, A480L and G134A;
(20) A466L, S473L and G134A;
(21) F113G, F114S, A466L, S473L, A480L, Q92A and G134A;
(22) F113G, F114S, A466L, S473L and G134A;
(23) A466L, S473L, A480L, Q92A, G134A;
(24) A466L, S473L, Q92A, G134A;
(25) F113G, F114S, Q92L, G134A;
(26) A466L, S473L, A480L, Q92L and G134A;
(27) A466L, S473L, Q92L and G134A;
(28) F113G, F114S, A466L, S473L, A480L, Q92A and G134L;
(29) A466L, S473L, A480L, Q92A and G134L;
(30) F113G, F114S, Q92C-G134C, A4661, S4731 and A480L;
(31) F113G, F114S, Q92C-G134C, A4661 and, S4731; and,
(32) A4661, S4731, A480L, Q92L and G134A.
E29. The mutant according to E1, wherein the mutant comprises the mutations A466L, S473L, A480L and G134A.
E30. The mutant according to E1, wherein the mutant comprises the mutations F113G, F114S, A466L, S473L and G134A.
E31. The mutant according to E1, wherein the mutant comprises the mutations F113G, F114S, A466L, S473L, A480L and G134A.
E32. The mutant according to E1, wherein the mutant comprises the mutations F113G, F114S, Q92C-G134C, A466L, S473L and A480L.
E33. The mutant according to E1 wherein
(a) the mutant comprises a leucine at position 466, 473 and 480 (466L, 473L and 480L) and an alanine at position 134 (134A) and wherein the mutant comprises a F1 polypeptide and a F2 polypeptide selected from the group consisting of:
   (1) a F2 polypeptide comprising the amino acid sequence of SEQ ID NO:255 and a F1 polypeptide comprising the amino acid sequence of SEQ ID NO:254;
   (2) a F2 polypeptide comprising an amino acid sequence that is at least 97%, 98% or 99% identical to the amino acid sequence of SEQ ID NO:255 and a F1 polypeptide comprising an amino acid sequence that is at least 97%, 98%, or 99% identical to the amino acid sequence of SEQ ID NO:254, or
(b) the mutant comprises a glycine (G) at position 113 (113G), a serine at position 114 (114S), a leucine at position 466 and 473 (466L and 473L) and an alanine at position 134 (134A) and wherein the mutant comprises a F1 polypeptide and a F2 polypeptide selected from the group consisting of:
   (1) a F2 polypeptide comprising the amino acid sequence of SEQ ID NO:291 and a F1 polypeptide comprising the amino acid sequence of SEQ ID NO:290;
   (2) a F2 polypeptide comprising an amino acid sequence that is at least 97%, 98% or 99% identical to the amino acid sequence of SEQ ID NO:291 and a F1 polypeptide comprising an amino acid sequence that is at least 97%, 98%, or 99% identical to the amino acid sequence of SEQ ID NO:290, (c) the mutant comprises a glycine (G) at position 113 (113G), a serine at position 114 (114S), a leucine at position 466, 473 and 480 (466L, 473L and 480L) and an alanine at position 134 (134A) and wherein the mutant comprises a F1 polypeptide and a F2 polypeptide selected from the group consisting of:
   (1) a F2 polypeptide comprising the amino acid sequence of SEQ ID NO:277 and a F1 polypeptide comprising the amino acid sequence of SEQ ID NO:276;
   (2) a F2 polypeptide comprising an amino acid sequence that is at least 97%, 98% or 99% identical to the amino acid sequence of SEQ ID NO:277 and a F1 polypeptide comprising an amino acid sequence that is at least 97%, 98%, or 99% identical to the amino acid sequence of SEQ ID NO:276, or,
(d) the mutant comprises a glycine (G) at position 113 (113G), a serine at position 114 (114S), a leucine at position 466, 473 and 480 (466L, 473L and 480L) and a cysteine at position 92 and 134 (92C and 134C) wherein the mutant comprises a F1 polypeptide and a F2 polypeptide selected from the group consisting of:
   (1) a F2 polypeptide comprising the amino acid sequence of SEQ ID NO:273 and a F1 polypeptide comprising the amino acid sequence of SEQ ID NO:272;
   (2) a F2 polypeptide comprising an amino acid sequence that is at least 97%, 98% or 99% identical to the amino acid sequence of SEQ ID NO:273 and a F1 polypeptide comprising an amino acid sequence that is at least 97%, 98%, or 99% identical to the amino acid sequence of SEQ ID NO:272.
E34. The mutant according to any one of E1 to E33, wherein the F1 polypeptide lacks the entire cytoplasmic domain.
E35. The mutant according to any one of E1 to E34, wherein the F1 polypeptide lacks the cytoplasmic domain and a portion of or all entire transmembrane domain. Preferably, the F1 polypeptide lacks the cytoplasmic domain and the transmembrane domain. Preferably, the F1 polypeptide comprises or consists of amino acid residues 113 to 477. Preferably, the F1 polypeptide comprises or consists of amino acid residues 113 to 480.
E36. The mutant according to any one of E1 to E33, wherein the F1 polypeptide comprises the ectodomain, the transmembrane domain and the cytoplasmic domain. In a preferred embodiment, the mutant comprises the full length F1 polypeptide and the full length F2 polypeptide.
E37. The mutant according to any one of E1 to E36, wherein the mutant is linked to a trimerization domain.
E38. The mutant according to E37, wherein the trimerization domain is a GCN4 leucine zipper or a phage T4 fibritin foldon.
E39. The mutant according to E38, wherein the trimerization domain is a phage T4 fibritin foldon.
E40. The mutant according to E39, wherein the trimerization domain is a phage T4 fibritin foldon of SEQ ID NO.7.
E41. The mutant according to any one of E37 to E40, wherein the trimerization domain is linked to the C-terminus of the F1 polypeptide.
E42. The mutant according to any one of E37 to E40, wherein the trimerization domain is linked to the C-terminus of the F1 polypeptide via a linker.
E43. The mutant according to E42, wherein the trimerization domain is linked to the C-terminus of the F1 polypeptide via a linker selected from the group consisting of GG, GS, GGGS or SAIG.
E44. The mutant according to E43, wherein the linker is GGGS.
E45. The mutant according to any one of E1 to E44, wherein the mutant is in the form of a trimer.
E46. The mutant according to any one of E1 to E45, wherein the mutant is in the prefusion conformation.
E47. The mutant according to any one of E1 to E45, wherein the mutant is in the prefusion conformation and specifically binds to an antibody (such as PIV1-8 mAb) specific for the PIV1 F ectodomain in the prefusion, but not postfusion, conformation.
E48. The mutant according to any one of E1 to E45, wherein the mutant is in the prefusion conformation and specifically binds to PIV1-8 mAb as measured by ELISA, preferably as disclosed in the Examples.
E49. The mutant according to any one of E1 to E45, which has increased stability as compared with the corresponding wild-type PIV1 F protein, wherein the stability is measured by binding of the mutant with antibody PIV1-8 mAb.
E50. The mutant of any one of E1 to E49 wherein the wild-type PIV1 F protein is SEQ ID NO:206.
E51. The mutant of any one of E1 to E49 wherein the wild-type PIV1 F protein is SEQ ID NO:207.
E52. The mutant of any one of E1 to E49 wherein the wild-type PIV1 F protein is SEQ ID NO:208.
E53. The mutant of any one of E1 to E49 wherein the wild-type PIV1 F protein is SEQ ID NO:209.
E54. The mutant of any one of E1 to E49 wherein the wild-type PIV1 F protein is SEQ ID NO:210.
E55. The mutant of any one of E1 to E49 wherein the wild-type PIV1 F protein is SEQ ID NO:211.
E56. The mutant of any one of E1 to E49 wherein the amino acid positions correspond to the amino acid sequence of a reference of SEQ ID NO:206.
E57. A nucleic acid comprising at least one coding sequence encoding at least one mutant of a wild-type PIV 1 F protein according to any one of embodiments E1-E56, preferably E36, or an immunogenic fragment or immunogenic variant thereof, wherein the nucleic acid comprises at least one heterologous untranslated region (UTR).
E58. A nucleic acid according to any one of the preceding embodiments, wherein the at least one heterologous untranslated region is selected from at least one heterologous 5′-UTR and/or at least one heterologous 3′-UTR.
E59. A nucleic acid according to any one of the preceding embodiments, wherein the at least one heterologous 3′-UTR comprises or consists of a nucleic acid sequence having at least, at most, exactly, or between any two of 99%, 98%, 97%, 96%, 95%, 90%, 85%, or 80% identity to CΨCGAGCΨGGΨACΨGCAΨGCA CGCAAΨGCΨÅ GCΨGCCCCΨΨΨCCCGΨCCΨG GGΨACCCCGA GΨCΨCCCCCG ACCΨCGGGΨC CCAGGΨAΨGC ΨCCCACCΨCC ACCYGCCCCA CYCACCACCY CΨGCΨAGΨΨC CAGACACCYC CCAAGCACGC AGCAAΨGCAG CΨCAAAACGC ΨΨAGCCΨAGC CACACCCCCA CGGGAAACAG CAGΨGAΨΨAA CCΨΨΨAGCAA ΨAAACGAAAG ΨΨΨAACΨAAG CΨAΨACΨAAC CCCAGGGΨΨG GΨCAAΨΨΨCG ΨGCCAGCCAC ACCCΨGGAGC YAGC.
E60. A nucleic acid according to any one of the preceding embodiments, wherein the at least one heterologous 5′-UTR comprises or consists of a nucleic acid sequence having at least, at most, exactly, or between any two of 99%, 98%, 97%, 96%, 95%, 90%, 85%, or 80% identity to GAAΨAAAC ΨAGΨAΨΨCΨΨCYGGYCCCCA CAGACYCAGA GAGAACCCGC CACC.
E61. A nucleic acid according to any one of the preceding embodiments, wherein the nucleic acid comprises at least one poly(A) sequence, preferably comprising 30 to 200 adenosine nucleotides and/or at least one poly(C) sequence, preferably comprising 10 to cytosine nucleotides.
E62. A nucleic acid according to any one of the preceding embodiments, wherein the nucleic acid is a DNA or an RNA.
E63. A nucleic acid according to any one of the preceding embodiments, wherein the nucleic acid is a coding RNA.
E64. A nucleic acid according to E63, wherein the coding RNA is an mRNA, a self-replicating RNA, a circular RNA, or a replicon RNA.
E65. A nucleic acid according to any one of the preceding embodiments, wherein the nucleic acid, preferably the coding RNA, is an mRNA.
E66. A nucleic acid according to E63, wherein the mRNA is not a replicon RNA or a self-replicating RNA.
E67. A nucleic acid according to any one of the preceding embodiments E63-E66, wherein the mRNA comprises at least one poly(A) sequence comprising 30 to 200 adenosine nucleotides and the 3′ terminal nucleotide is an adenosine.
E68. A nucleic acid according to any one of the preceding embodiments E62-E67, wherein the RNA, preferably the coding RNA, comprises a 5′-cap structure, preferably m7G, capO, cap1, cap2, a modified capO or a modified cap1 structure, preferably a 5′-cap1 structure.
E69. A nucleic acid according to any one of the preceding embodiments E62-E68, wherein the RNA is codon-optimized.
E70. A nucleic acid according to any one of the preceding embodiments E62-E69, wherein the RNA comprises a chemically modified nucleotide.
E71. A nucleic acid according to any one of the preceding embodiments E62-E70, wherein the RNA comprises 1-methylpseudouridine substitution. Preferably, all the uridines of the RNA are replaced by 1-methylpseudouridine.
E72. A nucleic acid according to any one of the preceding embodiments E62-E71, wherein the RNA is a purified RNA, preferably an RNA that has been purified by RP-HPLC and/or TFF.
E73. A nucleic according to any one of the preceding embodiments E62 to E72 wherein the RNA comprises the nucleic acid sequence of any of SEQ ID NO:403, SEQ ID NO:405, SEQ ID NO:407 and SEQ ID NO:409.
E74. A composition comprising at least one nucleic acid according to any one of the preceding embodiments E57-E73.
E75. A composition comprising at least one nucleic acid according to any one of the preceding embodiments E57-E73, wherein the composition comprises at least one pharmaceutically acceptable carrier.
E76. A composition comprising at least one nucleic acid according to any one of the preceding embodiments E57-E73, wherein the composition is a multivalent composition comprising a plurality or at least more than one of the nucleic acid according to any one of E57-E73.
E77. A composition comprising at least one nucleic acid according to any one of the preceding embodiments E57-E73, wherein the composition comprises RNA with an RNA integrity of 70% or more.
E78. A composition comprising at least one nucleic acid according to any one of the preceding embodiments E57-E73, wherein the composition comprises RNA with a capping degree of 70% or more, preferably wherein at least 70%, 80%, or 90% of the mRNA species comprise a Cap1 structure.
E79. A composition comprising at least one nucleic acid according to any one of the preceding embodiments E57-E73, wherein the at least one nucleic acid is complexed or associated with or at least partially complexed or partially associated with one or more cationic or polycationic compound, preferably cationic or polycationic polymer, cationic or polycationic polysaccharide, cationic or polycationic lipid, cationic or polycationic protein, cationic or polycationic peptide, or any combinations thereof.
E80. A composition comprising at least one nucleic acid according to any one of the preceding embodiments E57-E73, wherein the at least one nucleic acid is complexed or associated with one or more lipids or lipid-based carriers, thereby forming liposomes, lipid nanoparticles (LNP), lipoplexes, and/or nanoliposomes, preferably encapsulating the at least one nucleic acid.
E81. A composition comprising at least one nucleic acid according to any one of the preceding embodiments E57-E73, wherein the at least one nucleic acid is complexed with one or more lipids thereby forming lipid nanoparticles.
E82. A composition according to any one of the preceding embodiments E80-E81, wherein the LNP comprises a cationic lipid according to formula III-3:

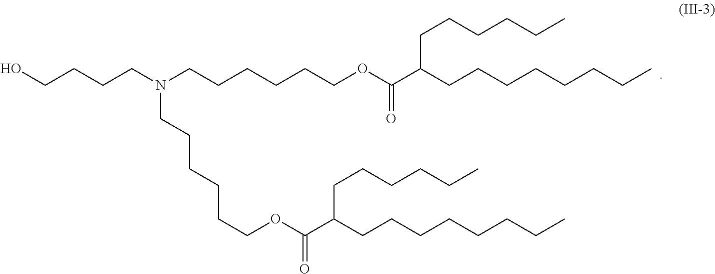 [SMILES: CCCCCCCCC(CCCCCC)C(=O)OCCCCCCN(CCCCO)CCCCCCOC(=O)C(CCCCCC)CCCCCCCC]

E83. A composition according to any one of the preceding embodiments E80-E82, wherein the LNP comprises a PEG lipid of formula (IVa):

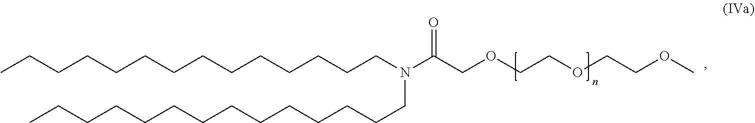

E84. A composition according to embodiment E83, wherein n has a mean value ranging from 30 to 60, preferably wherein n has a mean value of about 45, 46, 47, 48, 49, 50, 51, 52, 53, 54, most preferably wherein n has a mean value of 49 or 45.
E85. A composition according to any one of the preceding embodiments E80-E84, wherein the LNP comprises a PEG lipid of formula (IVa):

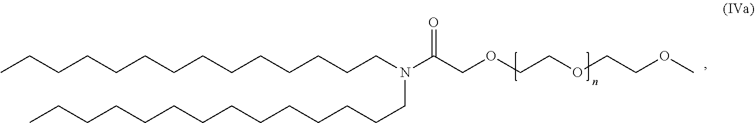

(Iva), wherein n is an integer selected such that the average molecular weight of the PEG lipid is about 2500 g/mol.
E86. A composition according to any one of the preceding embodiments E80-E85, wherein the LNP comprises one or more neutral lipids and/or one or more steroid or steroid analogues.
E87. A composition according to any one of the preceding embodiments E80-E86, wherein the neutral lipid is 1,2-distearoyl-sn-glycero-3-phosphocholine (DSPC), preferably wherein the molar ratio of the cationic lipid to DSPC is in the range from about 2:1 to about 8:1.
E88. A composition according to any one of the preceding embodiments E80-E87, wherein the steroid is cholesterol, preferably wherein the molar ratio of the cationic lipid to cholesterol is in the range from about 2:1 to about 1:1.
E89. A composition according to any one of the preceding embodiments E80-E88, wherein the LNP comprises (i) at least one cationic lipid, preferably a lipid of formula (III), more preferably lipid 111-3; (ii) at least one neutral lipid, preferably 1,2-distearoyl-sn-glycero-3-phosphocholine (DSPC); (iii) at least one steroid or steroid analogue, preferably cholesterol; and (iv) at least one polymer conjugated lipid, preferably a PEG-lipid derived from formula (IVa, with n=49), wherein (i) to (iv) are in a molar ratio of about 20-60% cationic lipid, 5-25% neutral lipid, 25-55% sterol, and 0.5-15% PEG-lipid.
E90. A composition according to any one of the preceding embodiments E80-E89, wherein the LNP comprises (i) at least one cationic lipid, preferably a lipid of formula (III), more preferably lipid 111-3; (ii) at least one neutral lipid, preferably 1,2-distearoyl-sn-glycero-3-phosphocholine (DSPC); (iii) at least one steroid or steroid analogue, preferably cholesterol; and (iv) at least one polymer conjugated lipid, preferably a PEG-lipid derived from formula (IVa, with n=45), wherein (i) to (iv) are in a molar ratio of about 20-60% cationic lipid, 5-25% neutral lipid, 25-55% sterol, and 0.5-15% PEG-lipid.
E91. A composition according to any one of the preceding embodiments E80-E90, wherein (i) to (iv) are in a molar ratio of about 50:10:38.5:1.5, preferably 47.5:10:40.8:1.7 or more preferably 47.4:10:40.9:1.7.
E92. A composition according to any one of the preceding embodiments E80-E91, wherein the nucleic acid is RNA and the composition comprises less than about 20% free (non complexed or non-encapsulated) RNA, preferably less than about 15% free RNA, more preferably less than about 10% free RNA.
E93. A composition according to any one of the preceding embodiments E80-E92, wherein the wt/wt ratio of lipid to nucleic acid is from about 10:1 to about 60:1, preferably from about 20:1 to about 30:1, for example about 25:1.
E94. A composition according to any one of the preceding embodiments E80-E93, wherein the n/p ratio of the LNPs encapsulating the nucleic acid is in a range from about 1 to about 10, preferably in a range from about 5 to about 7, more preferably about 6.
E95. A composition according to any one of the preceding embodiments E80-E94, wherein the composition has a polydispersity index (PDI) value of less than about 0.4, preferably of less than about 0.3, more preferably of less than about 0.2, most preferably of less than about 0.1.
E96. A composition comprise according to any one of the preceding embodiments E80-E95, wherein the LNPs have a Z-average size in a range of about 60 nm to about 120 nm, preferably less than about 120 nm, more preferably less than about 100 nm, most preferably less than about 80 nm.
E97. A composition according to any one of the preceding embodiments E80-E96, wherein the LNPs comprise less than about 10%, 9%, 8%, 7%, 6%, 5%, 4%, 3%, 2%, 1% LNPs that have a particle size exceeding about 500 nm.
E98. A composition according to any one of the preceding embodiments E80-E97, wherein the LNPs comprise less than about 10%, 9%, 8%, 7%, 6%, 5%, 4%, 3%, 2%, 1% LNPs that have a particle size smaller than about 20 nm.
E99. A composition according to any one of the preceding embodiments E80-E98, wherein the LNP comprises (i) at least one cationic lipid; (ii) at least one neutral lipid; (iii) at least one steroid or steroid analogue; and (iv) at least one PEG-lipid, wherein (i) to (iv) are in a molar ratio of about 20-60% cationic lipid, 5-25% neutral lipid, 25-55% sterol, and 0.5-15% PEG-lipid.
E100. A composition according to any one of the preceding embodiments E80-E99, wherein the LNP comprises (i) at least one cationic lipid according to formula III-3; (ii) DSPC; (iii) cholesterol; and (iv) a PEG-lipid, according to formula IVa, wherein (i) to (iv) are in a molar ratio of about 20-60% cationic lipid, 5-25% neutral lipid, 25-55% sterol, and 0.5-15% PEG-lipid. E101. A composition according to any one of the preceding embodiments E74-E100, wherein the composition is a lyophilized composition.
E102. An immunogenic composition comprising a mutant according to any one of E1 to E56, a nucleic acid according to any one of E57 to E73 or a composition according to any one of E73 to E101.
E103. An immunogenic composition according to embodiment E102, further comprising a hMPV A antigen selected from the group consisting of a mutant of a wild-type hMPV A F protein and a nucleic acid encoding a mutant of a wild-type hMPV A F protein. In one embodiment, the hMPV A antigen is selected from mutants of a wild-type hMPV A F protein and a nucleic acids encoding a mutant of a wild-type hMPV A F protein disclosed in any of WO16103238, WO20234300, WO21222639, WO22076669, WO22214678, WO23102373, WO23110618, WO23217988 and WO23102388. In one embodiment, the hMPV A antigen is a mutant of a wild-type hMPV A F protein or a nucleic acid encoding a mutant of a wild-type hMPV A F protein comprising the mutations of mutant 115-BV as disclosed in Battles et al, Nature communication 8:1528 (2017).
E104. An immunogenic composition according to embodiment E103, wherein the hMPV A antigen is a mutant of a wild-type hMPV A F protein.
E105. An immunogenic composition according to embodiment E103, wherein the hMPV A antigen is a mutant of a wild-type hMPV A F protein from the present disclosure, preferably from any of E1 to E72 of section B of the present disclosure.
E106. An immunogenic composition according to embodiment E103, wherein the hMPV A antigen comprises a nucleic acid encoding a mutant of a wild-type hMPV A F protein.
E107. An immunogenic composition according to embodiment E103, wherein the hMPV A antigen comprises a nucleic acid encoding a mutant of a wild-type hMPV A F protein from the present disclosure, preferably from any of E73 to E89 of section B of the present disclosure.
E108. An immunogenic composition according to any one of embodiments E102 to E107, further comprising a hMPV B antigen selected from the group consisting of a mutant of a wild-type hMPV B F protein and a nucleic acid encoding a mutant of a wild-type hMPV B F protein. In one embodiment, the hMPV B antigen is selected from mutants of a wild-type hMPV B F protein and nucleic acids encoding a mutant of a wild-type hMPV B F protein disclosed in any of WO16103238, WO20234300, WO21222639, WO22076669, WO22214678, WO23102373, WO23110618, WO23217988 and WO23102388. In one embodiment, the hMPV B antigen is a mutant of a wild-type hMPV B F protein or a nucleic acid encoding a mutant of a wild-type hMPV B F protein comprising the mutations of mutant 115-BV as disclosed in Battles et al, Nature communication 8:1528 (2017).
E109. An immunogenic composition according to embodiment E108, wherein the hMPV B antigen is a mutant of a wild-type hMPV B F protein.
E110. An immunogenic composition according to embodiment E108, wherein the hMPV B antigen is a mutant of a wild-type hMPV B F protein from the present disclosure, preferably from any of E1 to E72 of section B of the present disclosure.
E111. An immunogenic composition according to embodiment E108, wherein the hMPV B antigen comprises is a nucleic acid encoding a mutant of a wild-type hMPV B F protein.
E112. An immunogenic composition according to embodiment E108, wherein the hMPV B antigen comprises a nucleic acid encoding a mutant of a wild-type hMPV B F protein from the present disclosure, preferably from any of E73 to E89 of section B of the present disclosure.
E113. An immunogenic composition according to any one of embodiments E102 to E112, further comprising PIV3 antigen selected from the group consisting of a mutant of a wild-type PIV3 F protein and a nucleic acid encoding a mutant of a wild-type PIV3 F protein.
E114. An immunogenic composition according to embodiment E113, wherein the PIV3 antigen is a mutant of a wild-type PIV3 F protein.
E115. An immunogenic composition according to embodiment E113, wherein the PIV3 antigen is a mutant of a wild-type PIV3 F protein from the present disclosure, preferably from any of E1 to E52 of section D of the present disclosure.
E116. An immunogenic composition according to embodiment E113, wherein the PIV3 antigen is a mutant of a wild-type PIV3 F protein as disclosed in WO2018081289 or WO22207839.
E117. An immunogenic composition according to embodiment E113, wherein the PIV3 antigen comprises a nucleic acid encoding a mutant of a wild-type PIV3 F protein.
E118. An immunogenic composition according to embodiment E113, wherein the PIV3 antigen comprises a nucleic acid encoding a mutant of a wild-type PIV3 F protein from the present disclosure, preferably from any of E53 to E69 of section D of the present disclosure.
E119. An immunogenic composition according to embodiment E113, wherein the PIV3 antigen comprises a nucleic acid encoding a mutant of a wild-type PIV3 F protein as disclosed in WO2018081289 or WO2022207839.
E120. An immunogenic composition according to anyone of E102 to E119, further comprising an RSV antigen selected from the group consisting of a mutant of a wild-type RSV F protein of subtype A and a nucleic acid encoding a mutant of a wild-type RSV F protein of subtype A.
E121. An immunogenic composition according to embodiment E120, wherein the RSV antigen is a mutant of a wild-type RSV F protein of subtype A.
E122. An immunogenic composition according to embodiment E120, wherein the RSV antigen is a nucleic acid encoding a mutant of a wild-type RSV F protein of subtype A.
E123. An immunogenic composition according to embodiment E120, wherein the mutant of a wild-type RSV F protein of subtype A is disclosed in one of WO2009/079796, WO2010/149745, WO2011/008974, WO2014/160463, WO2014/174018, WO2014/202570, WO2015/013551, WO2015/177312, WO2017/005848, WO2017/174564, WO2017/005844, WO2017/109629, WO2022/002894 and WO2018/109220.
E124. An immunogenic composition according to any one of embodiments E102 to E123, further comprising an RSV antigen selected from the group consisting of a mutant of a wild-type RSV F protein of subtype B and a nucleic acid encoding a mutant of a wild-type RSV F protein of subtype B.
E125. An immunogenic composition according to embodiment E124, wherein the RSV antigen is a mutant of a wild-type RSV F protein of subtype B.
E126. An immunogenic composition according to embodiment E124, wherein the RSV antigen is a nucleic acid encoding a mutant of a wild-type RSV F protein of subtype B.
E127. An immunogenic composition according to embodiment E124, wherein the mutant of a wild-type RSV F protein of subtype B is disclosed in one of WO2009/079796, WO2010/149745, WO2011/008974, WO2014/160463, WO2014/174018, WO2014/202570, WO2015/013551, WO2015/177312, WO2017/005848, WO2017/174564, WO2017/005844, WO2017/109629, WO2022/002894 and WO2018/109220.

### 2. PIV1 F PROTEIN MUTANTS

In some aspects, the present invention provides mutants of wild-type PIV1 F proteins, wherein the mutants display introduced mutations in the amino acid sequence relative to the amino acid sequence of the corresponding wild-type PIV1 F protein and are immunogenic against the wild-type PIV1 F protein in the prefusion conformation or against a virus comprising the wild-type F protein. In certain embodiments, the PIV1 F mutants possess certain beneficial characteristics, such as increased immunogenic properties or improved stability in the prefusion conformation of the mutants or prefusion trimeric conformation of the mutant, as compared to the corresponding wild-type F protein. In still other embodiments, the present disclosure provides PIV1 F mutants that display one or more introduced mutations as described herein and bind to a prefusion specific antibody selected from PIV1-8 mAb.

The introduced amino acid mutations in the PIV1 F protein mutants include amino acid substitutions, deletions, or additions. In some embodiments, the only mutations in the amino acid sequence of the mutants are amino acid substitutions relative to a wild-type PIV1 F protein.

The amino acid sequence of a large number of native PIV1 F proteins from different strains, as well as nucleic acid sequences encoding such proteins, is known in the art. For example, the sequence of several PIV1 F0 precursor proteins are set forth in SeQ ID NOs:206 to 210.

The native PIV1 F protein exhibits remarkable sequence conservation across different strains.

In view of the substantial conservation of PIV1 F protein sequences, a person of ordinary skill in the art can easily compare amino acid positions between different native PIV1 F protein sequences to identify corresponding PIV1 F protein amino acid positions between different PIV1 strains. For example, across nearly all identified native PIV1 F0 precursor proteins, the protease cleavage site falls in the same amino acid positions. Thus, the conservation of native PIV1 F protein sequences across strains and subtypes allows use of a reference PIV1 F sequence for comparison of amino acids at particular positions in the PIV1 F protein. For the purposes of this disclosure (unless context indicates otherwise), the PIV1 F protein amino acid positions are given with reference to the sequence of the F0 precursor polypeptide set forth in SEQ ID NO: 206 (the amino acid sequence of the full length native F precursor polypeptide of the PIV1 strain; corresponding to Genbank Identifier AFP49460.1 (amino acids).

The consensus sequence for PIV1 (which correspond to SEQ ID NO: 206) was obtained as follows: Whole genome sequences for PIV1 were downloaded from NCBI's GenBank database as GenBank file format. Fusion protein gene sequences were filtered by sequence length to only include complete coding DNA sequence features. Translated fusion protein sequences were then parsed from GenBank file and saved as FASTA file. Muscle v5 was used to perform multiple sequence alignment of collected sequences. A Position specific score matrices (PSSMs) was generated to summarize the alignment information. For each column in the alignment, the number of each amino acid letters is counted and totaled. The consensus sequence at each position was calculated as the most common amino acid type in PSSM table. The final consensus sequence was then extracted and saved as FASTA file.

However, it should be noted, and one of skill in the art will understand, that different PIV1 F0 sequences may have different numbering systems, for example, if there are additional amino acid residues added or removed as compared to SEQ ID NO:206. As such, it is to be understood that when specific amino acid residues are referred to by their number, the description is not limited to only amino acids located at precisely that numbered position when counting from the beginning of a given amino acid sequence, but rather that the equivalent/corresponding amino acid residue in any and all PIV1 F sequences is intended even if that residue is not at the same precise numbered position, for example if the PIV1 sequence is shorter or longer than SEQ ID NO:206, or has insertions or deletions as compared to SEQ ID NO: 206.

### 2-1. Structure of the PIV1 F Protein Mutants

The PIV1 F protein mutants provided by the present disclosure comprise a F1 polypeptide and a F2 polypeptide. In several embodiments, the mutants further comprise a trimerization domain. In some embodiments, either the F1 polypeptide or the F2 polypeptide includes at least one introduced modification (e.g., amino acid substitution) as described in detail herein below. In some other embodiments, each of the F1 polypeptide and F2 polypeptide includes at least one introduced modification (e.g., amino acid substitution) as described in detail herein below.

2-1(a). F1 Polypeptide and F2 Polypeptide of the PIV1 F Mutants

In some embodiments, the mutants are in the mature form of the PIV1 F protein, which comprises two separate polypeptide chains, namely the F1 polypeptide and F2 polypeptide.

The F1 polypeptide chain of the mutant may be of the same length as the full length F1 polypeptide of the corresponding wild-type PIV1 F protein; however, it may also have deletions, such as deletions of 1 up to 36 amino acid residues from the C-terminus of the full-length F1 polypeptide. A full-length F1 polypeptide of the PIV1 F mutants corresponds to amino acid positions 113-555 of the native PIV1 F0 precursor, and includes (from N- to C-terminus) an extracellular region (residues 113 to 496), a transmembrane domain (residues 497-517), and a cytoplasmic domain (residues 518-555). It should be noted that amino acid residues 477 onwards in a native F1 polypeptide sequence are optional sequences in a F1 polypeptide of the PIV1 F mutants provided herein, and therefore may be absent from the F1 polypeptide of the mutant.

In some embodiments, the F1 polypeptide of the PIV1 F mutants lacks the entire cytoplasmic domain. In other embodiments, the F1 polypeptide lacks the cytoplasmic domain and a portion of or all entire transmembrane domain. In some specific embodiments, the mutant comprises a F1 polypeptide wherein the amino acid residues from position 477 through 555 are absent. Typically, for mutants that are linked to trimerization domain, such as a foldon, amino acids 477 through 555 can be absent. Thus, in some specific embodiment, amino acid residues 477 through 555 are absent from the F1 polypeptide of the mutant. In still other specific embodiments, the F1 polypeptide of the PIV1 F mutants comprises or consists of amino acid residues 103-477 of a native F0 polypeptide sequence, such as any of the F0 precursor sequence set forth in SEQ ID Nos: 206 to 210.

In some embodiments, the PIV1 F protein mutants comprise a mutation at position 480. In such case, the F1 polypeptide of the PIV1 F mutants comprises or consists of amino acid residues 103-480 of a native F0 polypeptide sequence.

On the other hand, the F1 polypeptide of the PIV1 F mutant may include a C-terminal linkage to a trimerization domain, such as a foldon. Many of the sequences of the PIV1 F mutants disclosed herein include a sequence of a PreScission cleavage site and Strep Tag II that are not essential for the function of the PIV1 F protein, such as for induction of an immune response. A person skilled in the art will recognize such sequences, and when appropriate, understand that these sequences are not included in a disclosed PIV1 F mutant.

In the PIV1 F mutants provided by the present disclosure, the F2 polypeptide chain may be of the same length as the full-length F2 polypeptide of the corresponding wild-type PIV1 F protein; it may also have deletions, such as deletions of 1, 2, 3, 4, 5, 6, 7, or 8 amino acid residues from the N-terminus or C-terminus of the F2 polypeptide.

The mutant in F0 form (i.e., a single chain polypeptide comprising the F2 polypeptide joined to the F1 polypeptide) or F1-F2 heterodimer form may form a protomer. The mutant may also be in the form of a trimer, which comprises three of the same protomer. Further, the mutants may be glycosylated proteins (i.e., glycoproteins) or non-glycosylated proteins. The mutant in F0 form may include, or may lack, the signal peptide sequence.

The F1 polypeptide and F2 polypeptide of the PIV1 F protein mutants to which one or more mutations are introduced can be from any wild-type PIV1 F proteins known in the art or discovered in the future, including, without limitations. In some embodiments, the PIV1 F mutant comprises a F1 and/or a F2 polypeptide from a PIV1 virus, from a known PIV1 F0 precursor protein such for example those set forth in any one of SeQ ID NOs: 206 to 210 to which one or more mutations are introduced.

In some embodiments, the PIV1 F protein mutants comprise a F1-polypeptide, a F2 polypeptide, and one or more introduced amino acid mutations as described herein below, wherein the F1 polypeptide comprises 350 consecutive amino acids and is at least 90, 95, 98, or 99 percent identical to amino acids 113-477 or 113-480 of any of the sequence of SEQ ID NO:206 to 210, wherein the F2 polypeptide comprises 70 consecutive amino acids and is at least 90, 95, 98, or 99 percent identical to amino acids 22-112 of any of the sequence of SEQ ID NO:206 to 210 and wherein PIV1 F protein mutant is stabilized in prefusion trimer conformation, whether as monomer or trimer.

2-1(b) Trimerization Domains

In several embodiments, the PIV1 F mutant provided by the present disclosure is linked to a trimerization domain. In some embodiments, the trimerization domain promotes the formation of trimer of three F1/F2 heterodimers.

Several exogenous trimerization domains that promote formation of stable trimers of soluble proteins are known in the art. Non limiting examples of such trimerization domains that can be linked to a mutant provided by the present disclosure include: (1) the GCN4 leucine zipper (Harbury et al. 1993 Science 262: 1401-1407); (2) the trimerization motif from the lung surfactant protein (Hoppe et al. 1994 FEB S Lett 344: 191-195); (3) collagen (McAlinden et al. 2003 Biol Chem 278:42200-42207); and (4) the phage T4 fibritin foldon (Miroshnikov et al. 1998 Protein Eng 11:329-414).

Typically, the trimerization domain is positioned C-terminal to the F1 polypeptide. It may join directly to the F1 polypeptide chain. Optionally, the multimerization domain is connected to the F1 polypeptide via a linker, such as an amino acid linker, for example the sequence GG, GS, GGGS, or SAIG. The linker can also be a longer linker (for example, including the repeat sequence GG). A preferred linker is GGGS. Numerous conformationally neutral linkers are known in the art that can be used in the mutants provided by the present disclosure. In some embodiments, the F mutant comprising a foldon domain include a protease cleavage site for removing the foldon domain from the F1 polypeptide, such as a thrombin site between the F1 polypeptide and the foldon domain.

In some embodiments, a foldon domain is linked to a F mutant at the C-terminus of F1 polypeptide. In specific embodiments, the foldon domain is a T4 fibritin foldon domain, such as the amino acid sequence GYIPEAPRDGQAYVRKDGEWVLLSTFL (SEQ ID NO: 7).

### 2-2. Introduced Mutations in the PIV1 F Protein Mutants

The PIV1 F mutants provided by the present disclosure comprise a F1 polypeptide and a F2 polypeptide, wherein (1) either the F1 polypeptide or (2) the F2 polypeptide, or (3) both the F1 polypeptide and F2 polypeptide include one or more introduced amino acid mutations relative to the amino acid sequence of the corresponding native F protein. The introduction of such amino acid mutations in the PIV1 F mutants confers a beneficial property to the mutants, such as enhanced immunogenicity, improved stability, improved expression or formation or improved stability of certain desired physical form or conformation of the mutants. Such introduced amino acid mutations are referred to as “engineered disulfide bond mutations,” “cavity filling mutations”, “proline substitution mutations”, “cleavage site mutation” or “glycine replacement mutation”.

The nature and purpose of “engineered disulfide bond mutations”, “cavity filling mutations”, “proline substitution mutations” and “glycine replacement mutation” are already disclosed above in connection with hMPV protein mutants.

The “cleavage site mutation” prevents cleavage of the PIV1 F protein mutants between amino acids 113 and 114. In such case, the F1 and F2 polypeptides form a single polypeptide instead of two separate polypeptides linked by disulfide bonds. An example of cleavage site mutation is F113G and F114S. PIV1 F protein mutants that include any additional mutations are also encompassed by the invention so long as the immunogenic property of the mutants is not substantially adversely affected by the additional mutations.

2-2(a) Engineered Disulfide Bond Mutations

In some embodiments, PIV1 protein F mutants provided by the present disclosure include one or more engineered disulfide bond mutations. The term “engineered disulfide bond mutation” refers to mutation of a pair of amino acid residues in a wild-type PIV1 F protein to a pair of cysteine residues. The introduced pair of cysteine residues allows for formation of a disulfide bond between the introduced cysteine residues, which disulfide bond serves to stabilize the protein's conformation or oligomeric state, such as prefusion conformation. For stabilizing the prefusion conformation of the mutant, the residue pairs for mutation to cysteine should be in close proximity in the prefusion conformation but distant in the post-fusion conformation. Preferably, the distance between the pair of residues (e.g. the beta carbons) is less than 8 Ain a prefusion conformation, but more than 20 Ain a post-fusion conformation.

In some embodiments, the PIV1 F protein mutants comprise only one engineered disulfide mutation (“single engineered disulfide mutation”). In some other embodiments, the PIV1 F protein mutants comprise at least two engineered disulfide mutations, wherein each pair of the cysteine residues of the engineered disulfide mutations are appropriately positioned when PIV1 F protein mutant is in prefusion conformation (“double engineered disulfide mutation”).

In some specific embodiments, the present disclosure provides a PIV1 F mutant comprising at least one engineered disulfide bond mutation, wherein the mutant comprises the same introduced mutations that are in any of the exemplary mutants provided in Tables 32, 36, and 37. The exemplary PIV1 F mutants provided in Tables 32, 36, and 37 are based on the same F0 sequence of PIV1 of SEQ ID NO:211. The same introduced mutations in each of the mutants can be made to a native F0 polypeptide sequence of any other PIV1 subtype or strain to arrive at different PIV1 F mutants, such as a native F0 polypeptide sequence set forth in any of the SeQ ID NOs: 206-210 or from any other PIV1 strain. PIV1 F mutants that are based on a native F0 polypeptide sequence of any other PIV1 subtype or strain and comprise any of the engineered disulfide mutations are also within the scope of the invention. In some particular embodiments, a PIV1 F protein mutant comprises at least one engineered disulfide mutation such as 92C and 134C, preferably Q92C-G134C.

2-2(b) Cavity Filling Mutations.

In other embodiments, the present disclosure provides PIV1 F mutants that comprise one or more cavity filling mutations. The term “cavity filling mutation” refers to the substitution of an amino acid residue in the wild-type PIV1 F protein by an amino acid that is expected to fill an internal cavity of the mature PIV1 F protein. In one application, such cavity-filling mutations contribute to stabilizing the prefusion conformation of a PIV1 F protein mutant. For example, the amino acids to be replaced for cavity-filling mutations typically include small aliphatic (e.g. Gly, Ala, and Val) or small polar amino acids (e.g. Ser and Thr). They may also include amino acids that are buried in the prefusion conformation, but exposed to solvent in the post-conformation. The replacement amino acids can aliphatic amino acids (Val, lie, Leu and Met), aromatic amino acid (His, Phe, Tyr and Trp), polar amino acids (Thr) with greater size than the replaced amino acids.

In some specific embodiments, a PIV1 F protein mutant comprises one or more cavity filling mutations at positions 198, 92, 466, 473 and 480, preferably 466, 473 and 480. In some specific embodiments, the present disclosure provides a PIV1 F mutant comprising one or more cavity filling mutations, wherein the mutant comprises the cavity filling mutations in any of the mutants provided in Tables 34 and 37. PIV1 F mutants provided in Tables 34 and 37 are based on same native F0 sequence of PIV1 of SEQ ID NO:211. The same introduced mutations in each of the mutants can be made to a native F0 polypeptide sequence of any other PIV1 subtype or strain to arrive at different PIV1 F mutants, such as a native F0 polypeptide sequence set forth in any of the SeQ ID NOs: 206-210 or from any other PIV1 strain. The PIV1 F mutants that are based on a native F0 polypeptide sequence of any other PIV1 subtype or strain and comprise any of the one or more cavity filling mutations are also within the scope of the invention. In some particular embodiments, a PIV1 F protein mutant provided by the present disclosure comprises at least one cavity filling mutation selected from the group consisting of: T198A, Q92A, Q92L, A466L, A466V, A4661, S473V, S473L, S4731, S473A, A480L and A480V.

In some particular embodiments, a PIV1 F protein mutant provided by the present disclosure comprises at least one cavity filling mutation selected from the group consisting of: A466L, S473L, A480L.

### 2-2 (c) Proline Substitution Mutations.

In still other embodiments, the present disclosure provides PIV1 F protein mutants that include one or more proline substitution mutations. The term proline substitution mutations” refers to the substitution of an amine acid by a proline to prevent the structural refolding that occurs during transit from the prefusion to post-fusion conformation

In some specific embodiments, the PIV1 F protein mutant comprises the proline mutation A128P. In some specific embodiments, the present disclosure provides a PIV1 F mutant comprising one or more proline substitution mutations provided in Tables 33. PIV F mutant provided in Table 33 is based on the native F0 sequence of PIV1 of SEQ ID NO:211. The same introduced mutation in the mutants can be made to a native F0 polypeptide sequence of any other PIV1 subtype or strain to arrive at different PIV1 F mutants, such as a native F0 polypeptide sequence set forth in any of the SeQ ID NOs: 206-210 or from any other PIV1 strain. PIV1 F mutants that are based on a native F0 polypeptide sequence of any other PIV1 subtype or strain and comprise any of the one or more promine substitution mutations are also within the scope of the invention. In some particular embodiments, the PIV1 F protein mutant comprises mutation A128P.

### 2-2 (d) Glycine Replacement Mutations.

In still other embodiments, the present disclosure provides PIV1 F protein mutants that include one or more glycine replacement mutation. The term “glycine replacement mutation” refers to the replacement of a glycine by another amino acid in the middle of an α-helix to improve protein stability, preferably an amino acid without Cβ substitution, such as Ala, Leu or Met. In some specific embodiments, the PIV1 F protein mutant comprises at least one glycine replacement mutation at position 134. In some specific embodiments, the present disclosure provides a PIV1 F mutant comprising one or more glycine replacement mutations, wherein the mutant comprises the glycine replacement mutation in the mutant provided in Table 35

PIV1 F mutants provided in Tables 35 is based on the native F0 sequence of PIV1 of SEQ ID NO:211. The same introduced mutations in each of the mutants can be made to a native F0 polypeptide sequence of any other PIV1 subtype or strain to arrive at different PIV1 F mutants, such as a native F0 polypeptide sequence set forth in any of the SeQ ID NOs: 206-210 or from any other PIV1 strain. PIV1 F mutants that are based on a native F0 polypeptide sequence of any other PIV1 subtype or strain and comprise any of the one or more glycine replacement mutations are also within the scope of the invention. In some particular embodiments, the PIV1 F protein mutant comprises mutation G134A or G134L, preferably G134A.

### 2-2 (e) Cleavage Site Mutation

The “cleavage site mutation” was introduced to prevent cleavage of the PIV1 F protein mutants between amino acids 112 and 113. However, it appeared that the PIV1 F protein mutants disclosed herein, when recombinantly expressed in CHO cells, were inefficiently cleaved between amino acids 112 and 113 even in the absence of any cleavage site mutation. As a result, the F1 and F2 polypeptides form a single polypeptide instead of two separate polypeptides linked by disulfide bonds. Unexpectedly, the “cleavage site mutation”, although not preventing cleavage which does not occur in the used expression system, provided some unexpected benefit in terms of thermal stability of the produced polypeptide.

In some specific embodiments, the present disclosure provides a PIV1 F mutant comprising one or more cleavage site mutations, wherein the mutant comprises the cleavage site mutation in the mutant provided in Table 37.

PIV1 F mutants provided in Tables 37 are based on the native F0 sequence of PIV1 of SEQ ID NO:211. The same introduced mutations in each of the mutants can be made to a native F0 polypeptide sequence of any other PIV1 subtype or strain to arrive at different PIV1 F mutants, such as a native F0 polypeptide sequence set forth in any of the SeQ ID NOs: 206-210 or from any other PIV1 strain. PIV1 F mutants that are based on a native F0 polypeptide sequence of any other PIV1 subtype or strain and comprise any of the one or more cleavage site mutations are also within the scope of the invention. In some particular embodiments, the PIV1 F protein mutant comprises the mutations F113G and F114S,

In some other particular embodiments, the present invention provides a PIV1 F mutant, wherein the mutant comprises a leucine at position 466, 473 and 480 (466L, 473L and 480L) and an alanine at position 134 (134A) wherein the mutant comprises a F1 polypeptide and a F2 polypeptide selected from the group consisting of:
(1) a F2 polypeptide comprising the amino acid sequence of SEQ ID NO:255 and a F1 polypeptide comprising the amino acid sequence of SEQ ID NO:254;
(2) a F2 polypeptide comprising an amino acid sequence that is at least 97%, 98% or 99% identical to the amino acid sequence of SEQ ID NO:255 and a F1 polypeptide comprising an amino acid sequence that is at least 97%, 98%, or 99% identical to the amino acid sequence of SEQ ID NO:254.

In some other particular embodiments, the present invention provides a PIV1 F mutant, wherein the mutant comprises a glycine (G) at position 113 (113G), a serine at position 114 (114S), a leucine at position 466 and 473 (466L and 473L) and an alanine at position 134 (134A) wherein the mutant comprises a F1 polypeptide and a F2 polypeptide selected from the group consisting of:
(1) a F2 polypeptide comprising the amino acid sequence of SEQ ID NO:291 and a F1 polypeptide comprising the amino acid sequence of SEQ ID NO:290;
(2) a F2 polypeptide comprising an amino acid sequence that is at least 97%, 98% or 99% identical to the amino acid sequence of SEQ ID NO:291 and a F1 polypeptide comprising an amino acid sequence that is at least 97%, 98%, or 99% identical to the amino acid sequence of SEQ ID NO:290.

In some other particular embodiments, the present invention provides a PIV1 F mutant, wherein the mutant comprises a glycine (G) at position 113 (113G), a serine at position 114 (114S), a leucine at position 466, 473 and 480 (466L, 473L and 480L) and an alanine at position 134 (134A) wherein the mutant comprises a F1 polypeptide and a F2 polypeptide selected from the group consisting of:
(1) a F2 polypeptide comprising the amino acid sequence of SEQ ID NO:277 and a F1 polypeptide comprising the amino acid sequence of SEQ ID NO:276;
(2) a F2 polypeptide comprising an amino acid sequence that is at least 97%, 98% or 99% identical to the amino acid sequence of SEQ ID NO:277 and a F1 polypeptide comprising an amino acid sequence that is at least 97%, 98%, or 99% identical to the amino acid sequence of SEQ ID NO:276.

In some other particular embodiments, the present invention provides a PIV1 F mutant, wherein the mutant comprises a glycine (G) at position 113 (113G), a serine at position 114 (114S), a leucine at position 466, 473 and 480 (466L, 473L and 480L) and a cysteine at position 92 and 134 (92C and 134C) wherein the mutant comprises a F1 polypeptide and a F2 polypeptide selected from the group consisting of:
(1) a F2 polypeptide comprising the amino acid sequence of SEQ ID NO:273 and a F1 polypeptide comprising the amino acid sequence of SEQ ID NO:272;
(2) a F2 polypeptide comprising an amino acid sequence that is at least 97%, 98% or 99% identical to the amino acid sequence of SEQ ID NO:273 and a F1 polypeptide comprising an amino acid sequence that is at least 97%, 98%, or 99% identical to the amino acid sequence of SEQ ID NO:272.

The PIV1 F protein mutants provided by the present disclosure can be prepared by routine methods known in the art, such as by expression in a recombinant host system using a suitable vector. Suitable recombinant host cells include, for example, insect cells, mammalian cells, avian cells, bacteria, and yeast cells. Examples of suitable insect cells include, for example, Sf9 cells, Sf21 cells, Tn5 cells, Schneider S2 cells, and High Five cells (a clonal isolate derived from the parental Trichoplusia ni BTI-TN-5B1-4 cell line (Invitrogen)). Examples of suitable mammalian cells include Chinese hamster ovary (CHO) cells, human embryonic kidney cells (HEK293 or Expi293 cells, typically transformed by sheared adenovirus type 5 DNA), NIH-3T3 cells, 293-T cells, Vero cells, and HeLa cells. Suitable avian cells include, for example, chicken embryonic stem cells (e.g., EBx® cells), chicken embryonic fibroblasts, chicken embryonic germ cells, quail fibroblasts (e.g. ELL-O), and duck cells. Suitable insect cell expression systems, such as baculovirus-vectored systems, are known to those of skill in the art and described in, e.g., Summers and Smith, Texas Agricultural Experiment Station Bulletin No. 1555 (1987). Materials and methods for baculovirus/insect cell expression systems are commercially available in kit form from, inter alia, Invitrogen, San Diego Calif. Avian cell expression systems are also known to those of skill in the art and described in, e.g., U.S. Pat. Nos. 5,340,740; 5,656,479; 5,830,510; 6,114,168; and 6,500,668. Similarly, bacterial and mammalian cell expression systems are also known in the art and described in, e.g., Yeast Genetic Engineering (Barr et al., eds., 1989) Butterworths, London.

A number of suitable vectors for expression of recombinant proteins in insect or mammalian cells are well-known and conventional in the art. Suitable vectors can contain a number of components, including, but not limited to one or more of the following: an origin of replication; a selectable marker gene; one or more expression control elements, such as a transcriptional control element (e.g., a promoter, an enhancer, a terminator), and/or one or more translation signals; and a signal sequence or leader sequence for targeting to the secretory pathway in a selected host cell (e.g., of mammalian origin or from a heterologous mammalian or non-mammalian species). For example, for expression in insect cells a suitable baculovirus expression vector, such as pFastBac (Invitrogen), is used to produce recombinant baculovirus particles. The baculovirus particles are amplified and used to infect insect cells to express recombinant protein. For expression in mammalian cells, a vector that will drive expression of the construct in the desired mammalian host cell (e.g., Chinese hamster ovary cells) is used.

The PIV1 F protein mutant polypeptides can be purified using any suitable methods. For example, methods for purifying PIV1 F protein mutant polypeptides by immunoaffinity chromatography are known in the art. Ruiz-Arguello et al., J. Gen. Virol., 85:3677-3687 (2004). Suitable methods for purifying desired proteins including precipitation and various types of chromatography, such as hydrophobic interaction, ion exchange, affinity, chelating, and size exclusion are well-known in the art. Suitable purification schemes can be created using two or more of these or other suitable methods. If desired, the PIV1 F protein mutant polypeptides can include a “tag” that facilitates purification, such as an epitope tag, a strep II tag or a histidine (HIS) tag. Such tagged polypeptides can conveniently be purified, for example from conditioned media, by chelating chromatography or affinity chromatography.

Below Table 4 provides representative sequences from PIV1 F0 polypeptide.

TABLE 4
F protein sequences from selected PIV1 strains.

| | SEQ | |
|---|---|---|
| Strain/ | ID | |
| GenBank_aa | NO | F0 protein sequence |
| HPIV1/WI/629- | 206 | MQSSEILLLVYSSLLLSSSLCQIPVDKLSNVGVIINEGKLLKIAGS |
| D00712/2009/ | | YESRYIVLSLVPSIDLQDGCGTTQIIQYKNLLNRLLIPLKDALDLQ |
| AFP49460.1 | | ESLITITNDTTVTNDNPQTRFFGAVIGTIALGVATAAQITAGIALA |
| | | EAREARKDIALIKDSIVKTHNSVEFIQRGIGEQIIALKTLQDFVND |
| | | EIRPAIGELRCETTALKLGIKLTQHYSELATAFSSNLGTIGEKSLT |
| | | LQALSSLYSANITEILSTIKKDKSDIYDIIYTEQVKGTVIDVDLEKY |
| | | MVTLLVKIPILSEIPGVLIYRASSISYNIEGEEWHVAIPNYIINKAS |
| | | SLGGADVTNCIESKLAYICPRDPTQLIPDNQQKCILGDVSKCPV |
| | | TKVINNLVPKFAFINGGVVANCIASTCTCGTNRIPVNQDRSKGV |
| | | TFLTYTNCGLIGINGIELYANKRGRDTTWGNQIIKVGPAVSIRPV |
| | | DISLNLASATNFLEESKTELMKARAIISAVGGWHNKESTQIIIIIIV |
| | | CILIIIICGILYYLYRVRRLLIMINSTNNSPINAYTLESRMKNPYMG |
| | | NHSN |
| HPIV1/Buenos | 207 | MQSSEVFLLVYSSLLLSSSLCQIPIDKLSNVGVIINEGKLLKIAGS |
| Aires/ARG/001/ | | YESRYIVLSLVPSIDLQDGCGTTQIIQYKNLLNRLLIPLKDALDLQ |
| 2016/ | | ESLITITNDTTVTNDNPQTRFFGAVIGTIALGVATAAQITAGIALA |
| MG773273.1 | | EAREARKDIALIKDSIVKTHNSVEFIQRGIGEQIIALKTLQDFVND |
| | | EIRPAIGELRCETTALKLGIKLTQHYSELATAFSSNLGTIGEKSLT |
| | | LQALSSLYSANITEILSTIKKDKSDIYDIIYTEQVKGTVIDVDLEKY |
| | | MVTLLVKIPILSEIPGVLIYRASSISYNIEGEEWHVAIPSYIINKAS |
| | | SLGGADVTNCIESKLAYICPRDPTQLIPDNQQKCILGDVSKCPV |
| | | TKVINNLVPKFAFINGGVVANCIASTCTCGTNRIPVNQDRSKGV |
| | | TFLTYTNCGLIGINGIELYANKRGRDTTWGNQIIKVGPAVSIRPV |
| | | DISLNLASATNFLEESKTELMRARAIISAVGGWHNKESTQIIIIIIV |
| | | CVLIIIICGILYYLYRVRSLLIMINSTNNSPINAYTLESRMKNPYMG |
| | | NHPN |
| Washington | 208 | MQKSEILFLVYSSLLLSSSLCQIPVEKLSNVGVIINEGKLLKIAGS |
| 1964/ | | YESRYIVLSLVPSIDLQDGCGTTQIIQYKNLLNRLLIPLKDALDLQ |
| NC_003461.1 | | ESLITITNDTTVTNDNPQTRFFGAVIGTIALGVATAAQITAGIALA |
| | | EAREARKDIALIKDSIVKTHNSVELIQRGIGEQIIALKTLQDFVND |
| | | EIRPAIGELRCETTALKLGIKLTQHYSELATAFSSNLGTIGEKSLT |
| | | LQALSSLYSANITEILSTTKKDKSDIYDIIYTEQVKGTVIDVDLEK |
| | | YMVTLLVKIPILSEIPGVLIYRASSISYNIEGEEWHVAIPNYIINKA |
| | | SSLGGADVTNCIESKLAYICPRDPTQLIPDNQQKCILGDVSKCP |
| | | VTKVINNLVPKFAFINGGVVANCIASTCTCGTNRIPVNQDRSRG |
| | | VTFLTYTNCGLIGINGIELYANKRGRDTTWGNQIIKVGPAVSIRP |
| | | VDISLNLASATNFLEESKTELMKARAIISAVGGWHNTESTQIIMIII |
| | | VCILIIIICGILYYLYRVRRLLVMINSTHNSPVNAYTLESRMRNPY |
| | | MGNNSN |
| HPIV1s/ | 209 | MQSSEILILVYSSLLLSSSLCQIPVDKLSNVGVIINEGKLLKIAGS |
| Zagreb.HR/50.13 | | YESRYIVLSLVPSIDLQDGCGTTQIIQYKNLLNRLLIPLKDALDLQ |
| (1833)/ | | ESLITITNDTTVTNDNPQTRFFGAVIGTIALGVATAAQITAGIALA |
| KT992807.1 | | EAREARKDIALIKDSIVKTHNSVEFIQRGIGEQIIALKTLQDFVND |
| | | EIRPAIGELRCETTALKLGIKLTQHYSELATAFSSNLGTIGEKSLT |
| | | LQALSSLYSANITEILSTIKKDKSDIYDIIYTEQVKGTVIDVDLEKY |
| | | MVTLLVKIPILSEIPGVLIYRASSISYNIEGEEWHVAIPNYIISKAS |
| | | SLGGADVTSCIESKLAYICPRDPTQLIPDNQQKCILGDVSKCPV |
| | | TKVINNLVPKFAFINGGVVANCIASTCTCGTNRIPVNQDRSKGV |
| | | TFLTYTNCGLIGINGIELYANKRGRDTTWGNQIIKVGPAVSIRPV |
| | | DISLNLASATNFLEESKTELMKARAIISAVGGWHNTESTQIIIIIIV |
| | | CILIIIICGILYYLYRVRRLLVMINSTNNSPINAYTLESRMRNPYM |
| | | GNHSN |
| HPIV1/FRA/ | 210 | MQSSEILILVYSSLLLSSSLCQIPVDKLSNVGVIINEGKLLKIAGS |
| 26503037/2006/ | | YESRYIVLSLVPSIDLQDGCGTTQIIQYKNLLNRLLIPLKDALDLQ |
| KF530208.1 | | ESLITITNDTTVTNDNPQTRFFGAVIGTIALGVATAAQITAGIALA |
| | | EAREARKDIALIKDSIVKTHNSVEFIQRGIGEQIIALKTLQDFVND |
| | | EIRPAIGELRCETTALKLGIKLTQHYSELATAFSSNLGTIGEKSLT |
| | | LQALSSLYSANITEILSTIKKDKSDIYDIIYTEQVKGTVIDVDLEKY |
| | | MVTLLVKIPILSEIPGVLIYRASSISYNIEGEEWHVAIPNYIISKAS |
| | | SLGGADVTNCIESKLAYICPRDPTQLIPDNQQKCILGDVSKCPV |
| | | TKVINNLVPKFAFINGGVVANCIASTCTCGTNRIPVNQDRSKGV |
| | | TFLTYTNCGLIGINGIELYANKRGRDTTWGNQIIKVGPAVSIRPV |
| | | DISLNLASATNFLEESKTELMKARAIISAVGGWHNTESTQIIIIIIV |
| | | CILIIIICGILYYLYRIRRLLVMINSTNNSPINAYTLESRMRNPYMG |
| | | NHSN |

Table 5 provides the amino acid sequence of F1 polypeptide without transmembrane and intracellular domains and F2 polypeptide of variants of mutant PIV1054 (based on F protein sequence from HPIV1/WI/629-D00712/2009 strain) to illustrate how a particular set of mutations applies to any PIV1 wild type F protein.

TABLE 5
Variants of Mutant PIV1054 and comprising introduced mutations A466L, S473L,
A480L, G134A

| | | | Amino Acid Sequence: |
|---|---|---|---|
| | Poly- | SEQ | (residues 113-480 for F1 polypeptide and |
| Mutant | peptide | ID | residues 22-112 for F2 polypeptide) |
| 054- | F1 | 366 | FFGAVIGTIALGVATAAQITAAIALAEAREARKDIALIKDSIV |
| HPIV1/WI/ | | | KTHNSVEFIQRGIGEQIIALKTLQDFVNDEIRPAIGELRCE |
| 629- | | | TTALKLGIKLTQHYSELATAFSSNLGTIGEKSLTLQALSSL |
| D00712/2009/ | | | YSANITEILSTIKKDKSDIYDIIYTEQVKGTVIDVDLEKYMVT |
| AFP49460.1 | | | LLVKIPILSEIPGVLIYRASSISYNIEGEEWHVAIPNYIINKAS |
| | | | SLGGADVTNCIESKLAYICPRDPTQLIPDNQQKCILGDVS |
| | | | KCPVTKVINNLVPKFAFINGGVVANCIASTCTCGTNRIPV |
| | | | NQDRSKGVTFLTYTNCGLIGINGIELYANKRGRDTTWGN |
| | | | QIIKVGPAVSIRPVDISLNLASLTNFLEELKTELMKL |
| | F2 | 367 | QIPVDKLSNVGVIINEGKLLKIAGSYESRYIVLSLVPSIDLQ |
| | | | DGCGTTQIIQYKNLLNRLLIPLKDALDLQESLITITNDTTVT |
| | | | NDNPQTR |
| 054- | F1 | 368 | FFGAVIGTIALGVATAAQITAAIALAEAREARKDIALIKDSIV |
| HPIV1/ | | | KTHNSVEFIQRGIGEQIIALKTLQDFVNDEIRPAIGELRCE |
| BuenosAires/ | | | TTALKLGIKLTQHYSELATAFSSNLGTIGEKSLTLQALSSL |
| ARG/ | | | YSANITEILSTIKKDKSDIYDIIYTEQVKGTVIDVDLEKYMVT |
| 001/2016/ | | | LLVKIPILSEIPGVLIYRASSISYNIEGEEWHVAIPSYIINKAS |
| MG773273.1 | | | SLGGADVTNCIESKLAYICPRDPTQLIPDNQQKCILGDVS |
| | | | KCPVTKVINNLVPKFAFINGGVVANCIASTCTCGTNRIPV |
| | | | NQDRSKGVTFLTYTNCGLIGINGIELYANKRGRDTTWGN |
| | | | QIIKVGPAVSIRPVDISLNLASLTNFLEELKTELMRL |
| | F2 | 369 | QIPIDKLSNVGVIINEGKLLKIAGSYESRYIVLSLVPSIDLQD |
| | | | GCGTTQIIQYKNLLNRLLIPLKDALDLQESLITITNDTTVTN |
| | | | DNPQTR |
| 054- | F1 | 370 | FFGAVIGTIALGVATAAQITAAIALAEAREARKDIALIKDSIV |
| Washington | | | KTHNSVELIQRGIGEQIIALKTLQDFVNDEIRPAIGELRCET |
| 1964/ | | | TALKLGIKLTQHYSELATAFSSNLGTIGEKSLTLQALSSLY |
| NC_003461.1 | | | SANITEILSTTKKDKSDIYDIIYTEQVKGTVIDVDLEKYMVT |
| | | | LLVKIPILSEIPGVLIYRASSISYNIEGEEWHVAIPNYIINKAS |
| | | | SLGGADVTNCIESKLAYICPRDPTQLIPDNQQKCILGDVS |
| | | | KCPVTKVINNLVPKFAFINGGVVANCIASTCTCGTNRIPV |
| | | | NQDRSRGVTFLTYTNCGLIGINGIELYANKRGRDTTWGN |
| | | | QIIKVGPAVSIRPVDISLNLASLTNFLEELKTELMKL |
| | F2 | 371 | QIPVEKLSNVGVIINEGKLLKIAGSYESRYIVLSLVPSIDLQ |
| | | | DGCGTTQIIQYKNLLNRLLIPLKDALDLQESLITITNDTTVT |
| | | | NDNPQTR |
| 054- | F1 | 372 | FFGAVIGTIALGVATAAQITAAIALAEAREARKDIALIKDSIV |
| HPIV1s/ | | | KTHNSVEFIQRGIGEQIIALKTLQDFVNDEIRPAIGELRCE |
| Zagreb.HR/50. | | | TTALKLGIKLTQHYSELATAFSSNLGTIGEKSLTLQALSSL |
| 13 | | | YSANITEILSTIKKDKSDIYDIIYTEQVKGTVIDVDLEKYMVT |
| (1833)/ | | | LLVKIPILSEIPGVLIYRASSISYNIEGEEWHVAIPNYIISKAS |
| KT992807.1 | | | SLGGADVTSCIESKLAYICPRDPTQLIPDNQQKCILGDVS |
| | | | KCPVTKVINNLVPKFAFINGGVVANCIASTCTCGTNRIPV |
| | | | NQDRSKGVTFLTYTNCGLIGINGIELYANKRGRDTTWGN |
| | | | QIIKVGPAVSIRPVDISLNLASLTNFLEELKTELMKL |
| | F2 | 373 | QIPVDKLSNVGVIINEGKLLKIAGSYESRYIVLSLVPSIDLQ |
| | | | DGCGTTQIIQYKNLLNRLLIPLKDALDLQESLITITNDTTVT |
| | | | NDNPQTR |
| 054- | F1 | 374 | FFGAVIGTIALGVATAAQITAAIALAEAREARKDIALIKDSIV |
| HPIV1/FRA/ | | | KTHNSVEFIQRGIGEQIIALKTLQDFVNDEIRPAIGELRCE |
| 26503037/2006/ | | | TTALKLGIKLTQHYSELATAFSSNLGTIGEKSLTLQALSSL |
| KF530208.1 | | | YSANITEILSTIKKDKSDIYDIIYTEQVKGTVIDVDLEKYMVT |
| | | | LLVKIPILSEIPGVLIYRASSISYNIEGEEWHVAIPNYIISKAS |
| | | | SLGGADVTNCIESKLAYICPRDPTQLIPDNQQKCILGDVS |
| | | | KCPVTKVINNLVPKFAFINGGVVANCIASTCTCGTNRIPV |
| | | | NQDRSKGVTFLTYTNCGLIGINGIELYANKRGRDTTWGN |
| | | | QIIKVGPAVSIRPVDISLNLASLTNFLEELKTELMKL |
| | F2 | 375 | QIPVDKLSNVGVIINEGKLLKIAGSYESRYIVLSLVPSIDLQ |
| | | | DGCGTTQIIQYKNLLNRLLIPLKDALDLQESLITITNDTTVT |
| | | | NDNPQTR |

### 3. Nucleic Acids Encoding PIV1 F Protein Mutants

In another aspect, the present invention provides nucleic acid molecules that encode a PIV1 F protein mutant described herein above. These nucleic acid molecules include DNA, cDNA, and RNA sequences. Nucleic acid molecules that encode only a F2 polypeptide or only a F1 polypeptide of a PIV1 F protein mutant are also encompassed by the invention. The nucleic acid molecule can be incorporated into a vector, such as an expression vector.

In some embodiments, the nucleic acid molecule encodes a precursor F0 polypeptide that, when expressed in an appropriate cell, is processed into a disclosed PIV1 F protein mutant. In some embodiments, the nucleic acid molecule encodes a precursor F0 polypeptide that, when expressed in an appropriate cell, is processed into a disclosed PIV1 F protein mutant, wherein the precursor F0 polypeptide includes, from N- to C-terminus, a signal peptide, a F2 polypeptide, and a F1 polypeptide. In some embodiments, the signal peptide comprises the amino acid sequence set forth as positions 1-21 of any one SEQ ID NOs: 206 to 210, wherein the amino acid positions correspond to the amino acid sequence of a reference of SEQ ID NO:206.

In a preferred embodiment, the nucleic acid is an RNA, more preferably an mRNA. In a preferred embodiment, the mRNA encodes a precursor F0 polypeptide that, when expressed in an appropriate cell, is processed into a full length PIV1 F protein mutant disclosed herein (i.e comprising one or more mutations, a full length F1 polypeptide and a full length F2 polypeptide). A full-length F1 polypeptide of the PIV1 F mutants corresponds to amino acid positions 113-555 of the native PIV1 F0 precursor, and includes (from N- to C-terminus) an extracellular region (residues 113 to 496), a transmembrane domain (residues 497-517), and a cytoplasmic domain (residues 518-555). In a preferred embodiment, the nucleic acid is an mRNA comprising a chemically modified nucleotide. In a preferred embodiment, the nucleic acid is an mRNA comprising a chemically modified nucleotide, preferably 1-methylpseudouridine. Preferably, all the uridines of the RNA are replaced by 1-methylpseudouridine.

In some embodiments, the nucleic acid molecule encodes a PIV1 F protein mutant selected from the group consisting of:
(1) a mutant comprising at least one engineered disulfide bond mutation;
(2) a mutant comprising at least one cavity filling mutation;
(3) a mutant comprising at least one proline substitution mutation;
(4) a mutant comprising at least one glycine replacement mutation;
(5) a mutant comprising at least one cleavage site mutation
(6) a mutant comprising a combination of at least one engineered disulfide mutation and at least one cavity filling mutation;
(7) a mutant comprising a combination of at least one engineered disulfide mutation and at least one proline substitution mutation;
(8) a mutant comprising a combination of at least one engineered disulfide mutation and a least one glycine replacement mutation;
(8) a mutant comprising a combination of at least one engineered disulfide mutation, at least one cavity filling mutation and at least one proline substitution mutation;
(10) a mutant comprising a combination of at least one engineered disulfide mutation, at least one cavity filling mutation, and a least one glycine replacement mutation;
(11) a mutant comprising a combination of at least one engineered disulfide mutation, at least one proline substitution mutation and a least one glycine replacement mutation;
(12) a mutant comprising a combination of at least one engineered disulfide mutation, at least one cavity filling mutation, at least one proline substitution mutation and a least one glycine replacement mutation
(13) a mutant comprising a combination of a cleavage site mutation and at least one engineered disulfide mutation;
(14) a mutant comprising a combination of a cleavage site mutation and at least one cavity filling mutation;
(15) a mutant comprising a combination of a cleavage site mutation and at least one proline substitution mutation;
(16) a mutant comprising a combination of a cleavage site mutation and at least one glycine replacement mutation;
(17) a mutant comprising a combination of a cleavage site mutation and at least one engineered disulfide mutation and at least one cavity filling mutation;
(18) a mutant comprising a combination of a cleavage site mutation and at least one engineered disulfide mutation and at least one proline substitution mutation;
(19) a mutant comprising a combination of a cleavage site mutation and at least one engineered disulfide mutation and a least one glycine replacement mutation;
(20) a mutant comprising a combination of a cleavage site mutation and at least one engineered disulfide mutation, at least one cavity filling mutation and at least one proline substitution mutation;
(21) a mutant comprising a combination of a cleavage site mutation and at least one engineered disulfide mutation, at least one cavity filling mutation, and a least one glycine replacement mutation;
(22) a mutant comprising a combination of a cleavage site mutation and at least one engineered disulfide mutation, at least one proline substitution mutation and a least one glycine replacement mutation;
(23) a mutant comprising a combination of a cleavage site mutation and at least one engineered disulfide mutation, at least one cavity filling mutation, at least one proline substitution mutation and a least one glycine replacement mutation;
(24) a mutant comprising a combination of a cleavage site mutation, at least one cavity filling mutation and at least one proline substitution mutation;
(25) a mutant comprising a combination of a cleavage site mutation, at least one cavity filling mutation and a least one glycine replacement mutation;
(26) a mutant comprising a combination of a cleavage site mutation, at least one proline substitution mutation and at least one glycine replacement mutation;
(27) a combination of at least one cavity filling mutation and at least one proline substitution mutation;
(28) a combination of at least one cavity filling mutation and a least one glycine replacement mutation (29) a combination of at least one proline substitution mutation and a least one glycine replacement mutation:
(30) a combination of at least one cavity filling mutation, at least one proline substitution mutation and a least one glycine replacement mutation.

In some specific embodiments, the present disclosure provides a nucleic acid molecule which encodes a mutant comprising the mutations selected from the group consisting of:
(1) Q92C-G134C;
(2) A466L;
(3) A466V;
(4) S473V;
(5) S473L;
(6) A480L;
(7) A466L and S473A;
(8) A466L and S473L;
(9) T198A;
(10) G134A;
(11) A128P;
(12) F113G, F114S, Q92C-G134C, A466L, S473L and A480L;
(13) Q92C-G134C, A466L, S473L and A480L;
(14) Q92C-G134C, A466L and S473L;
(15) F113G, F114S, Q92C-G134C, A466V, S473V and A480V;
(16) Q92C-G134C, A466V, S473V and A480V;
(17) Q92C-G134C, A466V and S473V;
(18) F113G, F114S, A466L, S473L, A480L and G134A;
(19) A466L, S473L, A480L and G134A;
(20) A466L, S473L and G134A;
(21) F113G, F114S, A466L, S473L, A480L, Q92A and G134A;
(22) F113G, F114S, A466L, S473L and G134A;
(23) A466L, S473L, A480L, Q92A, G134A;
(24) A466L, S473L, Q92A, G134A;
(25) F113G, F114S, Q92L, G134A;
(26) A466L, S473L, A480L, Q92L and G134A;
(27) A466L, S473L, Q92L and G134A;
(28) F113G, F114S, A466L, S473L, A480L, Q92A and G134L;
(29) A466L, S473L, A480L, Q92A and G134L;
(30) F113G, F114S, Q92C-G134C, A4661, S4731 and A480L;
(31) F113G, F114S, Q92C-G134C, A466 and, S4731; and,
(32) A4661, S4731, A480L, Q92L and G134A.

In some specific embodiments, the present disclosure provides a nucleic acid molecule, preferably a mRNA, more preferably a mRNA wherein all the uridines are replaced by 1-methylpseudouridine, said nucleic acid encoding a precursor F0 polypeptide that, when expressed in an appropriate cell, is processed into a full length PIV1 F protein mutant disclosed herein comprising the mutations selected from the group consisting of
(1) Q92C-G134C;
(2) A466L;
(3) A466V;
(4) S473V;
(5) S473L;
(6) A480L;
(7) A466L and S473A;
(8) A466L and S473L;
(9) T198A;
(10) G134A;
(11) A128P;
(12) F113G, F114S, Q92C-G134C, A466L, S473L and A480L;
(13) Q92C-G134C, A466L, S473L and A480L;
(14) Q92C-G134C, A466L and S473L;
(15) F113G, F114S, Q92C-G134C, A466V, S473V and A480V;
(16) Q92C-G134C, A466V, S473V and A480V;
(17) Q92C-G134C, A466V and S473V;
(18) F113G, F114S, A466L, S473L, A480L and G134A;
(19) A466L, S473L, A480L and G134A;
(20) A466L, S473L and G134A;
(21) F113G, F114S, A466L, S473L, A480L, Q92A and G134A;
(22) F113G, F114S, A466L, S473L and G134A;
(23) A466L, S473L, A480L, Q92A, G134A;
(24) A466L, S473L, Q92A, G134A;
(25) F113G, F114S, Q92L, G134A;
(26) A466L, S473L, A480L, Q92L and G134A;
(27) A466L, S473L, Q92L and G134A;
(28) F113G, F114S, A466L, S473L, A480L, Q92A and G134L;
(29) A466L, S473L, A480L, Q92A and G134L;
(30) F113G, F114S, Q92C-G134C, A4661, S4731 and A480L;
(31) F113G, F114S, Q92C-G134C, A4661 and, S4731; and,
(32) A4661, S4731, A480L, Q92L and G134A.

In some specific embodiments, the present disclosure provides a nucleic acid molecule, preferably a mRNA, more preferably a mRNA wherein all the uridines are replaced by 1-methylpseudouridine, said nucleic acid encoding a precursor F0 polypeptide that, when expressed in an appropriate cell, is processed into a full length PIV1 F protein mutant disclosed herein comprising the mutations F113G, F114S, Q92C-G134C, A466L, S473L and A480L.

### D. PIV3

The present disclosure relates to PIV3 F protein mutants, immunogenic compositions comprising the PIV3 F protein mutants, methods for producing the PIV3 protein mutants, compositions comprising the PIV3 F protein mutants, and nucleic acids that encode the PIV3 F protein mutants.

### 1. EXEMPLARY EMBODIMENTS (E) OF THE INVENTION

E1. A mutant of a wild-type PIV3 F protein, which mutant comprises a F1 polypeptide and a F2 polypeptide, wherein the mutant comprises at least one amino acid mutation relative to the amino acid sequence of the wild-type PIV3 F protein, and wherein the amino acid mutation is selected from the group consisting of:
(1) at least one engineered disulfide bond mutation;
(2) at least one cavity filling mutation;
(3) at least one proline substitution mutation;
(4) at least one glycine replacement mutation;
(5) an electrostatic mutation
(6) a combination of at least one engineered disulfide mutation and at least one cavity filling mutation;
(7) a combination of at least one engineered disulfide mutation and at least one proline substitution mutation;
(8) a combination of at least one engineered disulfide mutation and a least one glycine replacement mutation;
(8) a combination of at least one engineered disulfide mutation, at least one cavity filling mutation and at least one proline substitution mutation;
(10) a combination of at least one engineered disulfide mutation, at least one cavity filling mutation, and a least one glycine replacement mutation;
(11) a combination of at least one engineered disulfide mutation, at least one proline substitution mutation and a least one glycine replacement mutation;
(12) a combination of at least one engineered disulfide mutation, at least one cavity filling mutation, at least one proline substitution mutation and a least one glycine replacement mutation
(13) a combination of an electrostatic mutation and at least one engineered disulfide mutation;
(14) a combination of an electrostatic mutation and at least one cavity filling mutation;
(15) a combination of an electrostatic mutation and at least one proline substitution mutation;
(16) a combination of an electrostatic mutation and at least one glycine replacement mutation;
(17) a combination of an electrostatic mutation and at least one engineered disulfide mutation and at least one cavity filling mutation;
(18) a combination of an electrostatic mutation and at least one engineered disulfide mutation and at least one proline substitution mutation;
(19) a combination of an electrostatic mutation and at least one engineered disulfide mutation and a least one glycine replacement mutation;
(20) a combination of an electrostatic mutation and at least one engineered disulfide mutation, at least one cavity filling mutation and at least one proline substitution mutation;
(21) a combination of an electrostatic mutation and at least one engineered disulfide mutation, at least one cavity filling mutation, and a least one glycine replacement mutation;
(22) a combination of an electrostatic mutation and at least one engineered disulfide mutation, at least one proline substitution mutation and a least one glycine replacement mutation;
(23) a combination of an electrostatic mutation and at least one engineered disulfide mutation, at least one cavity filling mutation, at least one proline substitution mutation and a least one glycine replacement mutation;
(24) a combination of an electrostatic mutation, at least one cavity filling mutation and at least one proline substitution mutation;
(25) a combination of an electrostatic mutation, at least one cavity filling mutation and a least one glycine replacement mutation;
(26) a combination of an electrostatic mutation, at least one proline substitution mutation and at least one glycine replacement mutation;
(27) a cleavage site mutation, and,
(28) a cleavage site mutation in combination with the mutation or combination of mutations listed in above items (1) to (26).
E2. The mutant according to E1 wherein the mutant comprises an engineered disulfide mutation selected from the group consisting of V175C-A202C, S160C-V170C, E209C-L234C, E209C-S233C, G85C-E209C and Q162C-L168C.
E3. The mutant according to E2 wherein the engineered disulfide mutation is V175C-A202C or Q162C-L168C.
E4. The mutant according to E2 wherein the engineered disulfide mutation is S160C-V170C.
E5. The mutant according to E2 wherein the engineered disulfide mutation is E209C-L234C.
E6. The mutant according to E2 wherein the mutant comprises two engineered disulfide mutations selected from the group consisting of V175C-A202C, S160C-V170C, E209C-L234C and Q162C-L168C, preferably S160C-V170C and E209C-L234C.
E7. The mutant according to any one of E1 to E6, wherein the mutant comprises a cavity filling mutation.
E8. The mutant according to E7, wherein the cavity filling mutation is selected from the group consisting of T277V, S470A, S470L, S477A, A463L, 1474F and 1474Y.
E9. The mutant according to E7, wherein the cavity filling mutation is selected from the group consisting of S470A, 1474F S477A and A463L.
E10. The mutant according to E8, wherein the cavity filling mutation is S470A or S470L.
E11. The mutant according to E8, wherein the cavity filling mutation is S477A.
E12. The mutant according to E8, wherein the cavity filling mutation is A463L.
E13. The mutant according to E4, wherein the cavity filling mutation is 1474Y or 1474F.
E14. The mutant according to any one of E1 to E13, wherein the mutant comprises two or three cavity filling mutations selected from S470A, S470L, S477A, A463L, 1474F and 1474Y.
E15. The mutant according to E14, wherein the cavity filling mutations are S470A and S477A.
E16. The mutant according to E14, wherein the cavity filling mutations are A463L and 1474F, A463L and S470L or, A463L and 1474F.
E17. The mutant according to any one of E1 to E16, wherein the mutant comprises a proline substitution mutation.
E18. The mutant according to E17, wherein the proline substitution mutation is S164P or G219P.
E19. The mutant according to any one of E1 to E18, wherein the mutant comprises a cleavage site mutation.
E20. The mutant according to E19, wherein the cleavage site mutation comprises F110G and F111S.
E21. The mutant according to any one of E1 to E20, wherein the mutant comprises a glycine replacement mutation.
E22. The mutant according to E21, wherein the glycine replacement mutation is G196 Aor G230A.
E23. The mutant according to E21, wherein the glycine replacement mutation is G196A.
E24. The mutant according to E21, wherein the glycine replacement mutation is G230A.
E25. The mutant according to any one of E1 to E24, wherein the mutant comprises an electrostatic mutation.
E26. The mutant according to E25, wherein the electrostatic mutation is E182L or D455S.
E27. The mutant according to E1, wherein the mutant comprises the mutations selected from the group consisting of:
(1) V175C and A202C;
(2) S160C and V170C;
(3) S164P;
(4) G196A;
(5) G219P;
(6) G230A;
(7) E182L;
(8) S470A;
(9) S477A;
(10) S470A and S477A;
(11) D455S;
(12) A463L;
(13) Q162C, L168C, S470A and S477A;
(14) S160C, V170C, S470A and S477A;
(15) G230A, S470A and S477A;
(16) A463L, S470A and S477A;
(17) E209C and L234C;
(18) A463L and S470L;
(19) S160C, V170C, E209C, L234C, A463L and S470L;
(20) S160C, V170C, E209C, L234C, A463L and 1474F;
(21) S160C, V170C, E209C, L234C, A463L, S470L, F110G, F111S;
(22) S160C, V170C, A463L and S470L;
(23) Q162C, L168C, G230A, A463V and 1474Y;
(24) Q162C, L168C, G230A, S470A and S477A;
(25) Q162C, L168C, G230A and A463L;
(26) Q162C, L168C, G230A, A463L, S470A and S477A;
(27) S160C, V170C, G230A, A463V and 1474Y;
(28) S160C, V170C, G230A, S470A and S477A;
(29) S160C, V170C, G230A and A463L;
(30) S160C, V170C, G230A, A463L, S470A and S477A;
(31) S160C, V170C and A463L;
(32) E209C and S233C;
(33) G85C and E209C;
(34) T277V;
(35) A463L and 1474F;
(36) A4631, S470I (37) S160C, V170C, E209C, S233C, A463L and S470L;
(38) S160C, V170C, E209C, S233C, A463L and 1474F;
(39) S160C, V170C, G85C, E209C, A463L and S470L;
(40) S160C, V170C, G85C, E209C, A463L and 1474F;
(41) S160C, V170C, E209C, L234C, T277V, A463L and S470L;
(42) S160C, V170C, E209C, L234C, T277V, A463L and 1474F;
(43) S160C, V170C, E209C, S233C, T277V, A463L and S470L;
(44) S160C, V170C, E209C, S233C, T277V, A463L and 1474F;
(45) S160C, V170C, G85C, E209C, T277V, A463L and 1474F;
(46) S160C, V170C, E209C, L234C, D455S, A463L and S470L;
(47) S160C, V170C, E209C, S233C, D455S, A463L and S470L;
(48) S160C, V170C, G85C, E209C, D455S, A463L and S470L;
(49) S160C, V170C, E209C, L234C, T277V, D455S, A463L and S470L;
(50) S160C, V170C, E209C, S233C, T277V, D455S, A463L and S470L;
(51) S160C, V170C, G85C, E209C, T277V, D455S, A463L and S470L;
(52) S160C, V170C and S470L;
(53) R106G, T107S, E108A, R109S, S160C, V170C, E209C, L234C, A463L and S470L;
(54) R106G, T107S, E108A, R109S, S160C, V170C, E209C, S233C, A463L and S470L;
(55) R106G, T107S, E108A, R109S, S160C, V170C, G85C, E209C, A463L and S470L;
(56) F110G, F111S, S160C, V170C, E209C, L234C, A463L and S470L;
(57) F110G, F111S, S160C, V170C, E209C, S233C, A463L and S470L;
(58) F110G, F111S, S160C, V170C, A463L and S470L;
(59) F110G, F111S, S160C, V170C and S470L;
(60) S160C, V170C, A463L and S477L;
(61) S160C, V170C, E209C, L234C, A463L and S470L; and,
(62) S160C, V170C and S470L.
E28. The mutant according to E1, wherein the mutant comprises the mutations selected from the group consisting of:
(1) G230A, S470A and S477A;
(2) S160C, V170C, G230A and A463L;
(3) S160C, V170C, S470A and S477A;
(4) S160C, V170C, G230A, S470A and S477A;
(5) S160C, V170C, G230A, A463L, S470A and S477 Å
(6) S160C, V170C, E209C, L234C, A463L and S470L;
(7) S160C, V170C, E209C, L234C, A463L and 1474F;
(8) S160C, V170C, E209C, L234C, A463L, S470L, F110G, F111S; and,
(9) S160C, V170C, A463L and S470L, and,
(10) E209C and L234C.
E29. The mutant according to E1, wherein the mutant comprises the mutations selected from S160C, V170C, A463L and S470L and the F1 polypeptide comprises or consists of amino acid residues 110 to 484.
E30. The mutant according to any one of E1 to E29 wherein the mutant further comprises the mutations selected from substitution of the amino acid R106G, T107S, E108 Aand R109S.
E31. The mutant according to E1, wherein
(a) the mutant comprises an alanine at position 230, 470 and 477 (230A, 470A and 477A) wherein the mutant comprises a F1 polypeptide and a F2 polypeptide selected from the group consisting of:
   (1) a F2 polypeptide comprising the amino acid sequence of SEQ ID NO:329 and a F1 polypeptide comprising the amino acid sequence of SEQ ID NO:328;
   (2) a F2 polypeptide comprising an amino acid sequence that is at least 97%, 98% or 99% identical to the amino acid sequence of SEQ ID NO:329 and a F1 polypeptide comprising an amino acid sequence that is at least 97%, 98%, or 99% identical to the amino acid sequence of SEQ ID NO:328; or,
(b) the mutant comprises a cysteine at position 160 (160C) and 170 (170C), a leucine at position 463 (463L) and an alanine at position 230 (230A) wherein the mutant comprises a F1 polypeptide and a F2 polypeptide selected from the group consisting of:
   (1) a F2 polypeptide comprising the amino acid sequence of SEQ ID NO:353 and a F1 polypeptide comprising the amino acid sequence of SEQ ID NO:352;
   (2) a F2 polypeptide comprising an amino acid sequence that is at least 97%, 98% or 99% identical to the amino acid sequence of SEQ ID NO:353 and a F1 polypeptide comprising an amino acid sequence that is at least 97%, 98%, or 99% identical to the amino acid sequence of SEQ ID NO:352; or,
(c) the mutant comprises a cysteine at position 160 (160C) and 170 (170C) and an alanine at position 470 (470A) and 477 (477A) wherein the mutant comprises a F1 polypeptide and a F2 polypeptide selected from the group consisting of:
(1) a F2 polypeptide comprising the amino acid sequence of SEQ ID NO:339 and a F1 polypeptide comprising the amino acid sequence of SEQ ID NO:338;
(2) a F2 polypeptide comprising an amino acid sequence that is at least 97%, 98% or 99% identical to the amino acid sequence of SEQ ID NO:339 and a F1 polypeptide comprising an amino acid sequence that is at least 97%, 98%, or 99% identical to the amino acid sequence of SEQ ID NO:338; or,
(d) the mutant comprises a cysteine at position 160 (160C) and 170 (170C) and an alanine at position 230 (230A), 470 (470A) and 477 (477A) wherein the mutant comprises a F1 polypeptide and a F2 polypeptide selected from the group consisting of:
   (1) a F2 polypeptide comprising the amino acid sequence of SEQ ID NO:351 and a F1 polypeptide comprising the amino acid sequence of SEQ ID NO:350;
   (2) a F2 polypeptide comprising an amino acid sequence that is at least 97%, 98% or 99% identical to the amino acid sequence of SEQ ID NO:351 and a F1 polypeptide comprising an amino acid sequence that is at least 97%, 98%, or 99% identical to the amino acid sequence of SEQ ID NO:350; or,
(e) the mutant comprises a cysteine at position 160 (160C) and 170 (170C), a leucine at position 463 (463L) and an alanine at position 230 (230A), 470 (470A) and 477 (477A) wherein the mutant comprises a F1 polypeptide and a F2 polypeptide selected from the group consisting of:
   (1) a F2 polypeptide comprising the amino acid sequence of SEQ ID NO:355 and a F1 polypeptide comprising the amino acid sequence of SEQ ID NO:354;
   (2) a F2 polypeptide comprising an amino acid sequence that is at least 97%, 98% or 99% identical to the amino acid sequence of SEQ ID NO:355 and a F1 polypeptide comprising an amino acid sequence that is at least 97%, 98%, or 99% identical to the amino acid sequence of SEQ ID NO:354.
E32. The mutant according to E1, wherein
(a) the mutant comprises a cysteine at position 160 (160C), 170 (170C), 209 (209C) and 234 (234C) and a leucine at position 463 (463L) and 470 (470L), wherein the mutant comprises a F1 polypeptide and a F2 polypeptide selected from the group consisting of:
   (1) a F2 polypeptide comprising the amino acid sequence of SEQ ID NO:438 and a F1 polypeptide comprising the amino acid sequence of SEQ ID NO:437;
   (2) a F2 polypeptide comprising an amino acid sequence that is at least 97%, 98% or 99% identical to the amino acid sequence of SEQ ID NO:438 and a F1 polypeptide comprising an amino acid sequence that is at least 97%, 98%, or 99% identical to the amino acid sequence of SEQ ID NO:437; or,
(b) the mutant comprises a cysteine at position 160 (160C), 170 (170C), 209 (209C) and 234 (234C) and a leucine at position 463 (463L) and a phenylalanine at position 474 (474F), wherein the mutant comprises a F1 polypeptide and a F2 polypeptide selected from the group consisting of:
   (1) a F2 polypeptide comprising the amino acid sequence of SEQ ID NO:440 and a F1 polypeptide comprising the amino acid sequence of SEQ ID NO:439;
   (2) a F2 polypeptide comprising an amino acid sequence that is at least 97%, 98% or 99% identical to the amino acid sequence of SEQ ID NO:440 and a F1 polypeptide comprising an amino acid sequence that is at least 97%, 98%, or 99% identical to the amino acid sequence of SEQ ID NO:439; or,
(c) the mutant comprises a cysteine at position 160 (160C), 170 (170C), 209 (209C) and 234 (234C), a leucine at position 463 (463L) and 470 (470L), a glycine at position 110 (110G) and a serine at position 111 (111S) wherein the mutant comprises a F1 polypeptide and a F2 polypeptide selected from the group consisting of:
   (1) a F2 polypeptide comprising the amino acid sequence of SEQ ID NO:482 and a F1 polypeptide comprising the amino acid sequence of SEQ ID NO:481;
   (2) a F2 polypeptide comprising an amino acid sequence that is at least 97%, 98% or 99% identical to the amino acid sequence of SEQ ID NO:482 and a F1 polypeptide comprising an amino acid sequence that is at least 97%, 98%, or 99% identical to the amino acid sequence of SEQ ID NO:481; or,
(d) the mutant comprises a cysteine at position 160 (160C) and 170 (170C) and a leucine at position 463 (463L) and 470 (470L), wherein the mutant comprises a F1 polypeptide and a F2 polypeptide selected from the group consisting of:
   (1) a F2 polypeptide comprising the amino acid sequence of SEQ ID NO:494 and a F1 polypeptide comprising the amino acid sequence of SEQ ID NO:493;
   (2) a F2 polypeptide comprising an amino acid sequence that is at least 97%, 98% or 99% identical to the amino acid sequence of SEQ ID NO:494 and a F1 polypeptide comprising an amino acid sequence that is at least 97%, 98%, or 99% identical to the amino acid sequence of SEQ ID NO:493.
E33. The mutant according to any one of E1 to E32, wherein the F1 polypeptide lacks the entire cytoplasmic domain or wherein the F1 polypeptide lacks the cytoplasmic domain and a portion of or all entire transmembrane domain.
E34. The mutant according to any one of E1 to E30, wherein the F1 polypeptide lacks the cytoplasmic domain and the transmembrane domain E35. The mutant according to any one of E1 to E33, wherein the F1 polypeptide comprises or consists of amino acid residues 110 to 481.
E36. The mutant according to any one of E1 to E33, wherein the F1 polypeptide comprises or consists of amino acid residues 110 to 484.
E37. The mutant according to any one of E1 to E31, wherein the F1 polypeptide comprises the ectodomain, the transmembrane domain and the cytoplasmic domain. In a preferred embodiment, the mutant comprises the full length F1 polypeptide and the full length F2 polypeptide.
E38. The mutant according to any one of E1 to E37, wherein the mutant is linked to a trimerization domain.
E39. The mutant according to E38, wherein the trimerization domain is a GCN4 leucine zipper or a phage T4 fibritin foldon.
E40. The mutant according to E39, wherein the trimerization domain is a phage T4 fibritin foldon.
E41. The mutant according to E40, wherein the trimerization domain is a phage T4 fibritin foldon of SEQ ID NO.7.
E42. The mutant according to any one of E38 to E41, wherein the trimerization domain is linked to the C-terminus of the F1 polypeptide.
E43. The mutant according to any one of E38 to E42, wherein the trimerization domain is linked to the C-terminus of the F1 polypeptide via a linker.
E44. The mutant according to E43, wherein the trimerization domain is linked to the C-terminus of the F1 polypeptide via a linker selected from the group consisting of GG, GS, GGGS or SAIG.
E45. The mutant according to E44, wherein the linker is GGGS.
E46. The mutant according to any one of E1 to E45, wherein the mutant is in the form of a trimer.
E47. The mutant according to any one of E1 to E46, wherein the mutant is in the prefusion conformation.
E48. The mutant according to any one of E1 to E46, wherein the mutant is in the prefusion conformation and specifically binds to an antibody (such as PIA174 mAb) specific for the PIV3 F ectodomain in the prefusion, but not postfusion, conformation.
E49. The mutant according to any one of E1 to E47, wherein the mutant is in the prefusion conformation and specifically binds to PIA174 mAb as measured by ELISA, preferably as disclosed in the Examples.
E50. The mutant according to any one of E1 to E46, which has increased stability as compared with the corresponding wild-type PIV3 F protein, wherein the stability is measured by binding of the mutant with antibody PIA174 mAb.
E51. The mutant of any one of E1 to E46 wherein the wild-type PIV3 F protein is SEQ ID NO:300, SEQ ID NO:301, SEQ ID NO:302, SEQ ID NO:303 or SEQ ID NO:304. Preferably the wild-type PIV3 F protein is SEQ ID NO:300.
E52. The mutant of any one of E1 to E51 wherein the amino acid positions correspond to the amino acid sequence of a reference of SEQ ID NO:300
E53. A nucleic acid comprising at least one coding sequence encoding at least one mutant of a wild-type PIV3 F protein according to any one of embodiments E1-E52, preferably E37, or an immunogenic fragment or immunogenic variant thereof, wherein the nucleic acid comprises at least one heterologous untranslated region (UTR).
E54. A nucleic acid according to E53, wherein the at least one heterologous untranslated region is selected from at least one heterologous 5′-UTR and/or at least one heterologous 3′-UTR.
E55. A nucleic acid according to any one of the preceding embodiments, wherein the at least one heterologous 3′-UTR comprises or consists of a nucleic acid sequence having at least, at most, exactly, or between any two of 99%, 98%, 97%, 96%, 95%, 90%, 85%, or 80% identity to CΨCGAGCΨGGΨACΨGCAΨGCA CGCAAYGCYA GCYGCCCCΨΨΨCCCGYCCYG GGYACCCCGA GYCYCCCCCG ACCYCGGGYC CCAGGYAYGC YCCCACCYCC ACCYGCCCCA CYCACCACCY CYGCYAGYYC CAGACACCΨC CCAAGCACGC AGCAAΨGCAG CΨCAAAACGC ΨΨAGCCΨAGC CACACCCCCA CGGGAAACAG CAGΨGAΨΨAA CCΨΨΨAGCAA ΨAAACGAAAG ΨΨΨAACΨAAG CΨAΨACΨAAC CCCAGGGΨΨG GΨCAAΨΨΨCG ΨGCCAGCCAC ACCCΨGGAGC ΨAGC.
E56. A nucleic acid according to any one of the preceding embodiments, wherein the at least one heterologous 5′-UTR comprises or consists of a nucleic acid sequence having at least, at most, exactly, or between any two of 99%, 98%, 97%, 96%, 95%, 90%, 85%, or 80% identity to GAAΨA AAC ΨA GΨAΨΨCΨΨCΨGGΨCCCCA CAGACΨCAGA GAGAACCCGC CACC.
E57. A nucleic acid according to any one of the preceding embodiments, wherein the nucleic acid comprises at least one poly(A) sequence, preferably comprising 30 to 200 adenosine nucleotides and/or at least one poly(C) sequence, preferably comprising 10 to cytosine nucleotides.
E58. A nucleic acid according to any one of the preceding embodiments, wherein the nucleic acid is a DNA or an RNA.
E59. A nucleic acid according to any one of the preceding embodiments, wherein the nucleic acid is a coding RNA.
E60. A nucleic acid according to E59, wherein the coding RNA is an mRNA, a self-replicating RNA, a circular RNA, or a replicon RNA.
E61. A nucleic acid according to any one of the preceding embodiments, wherein the nucleic acid, preferably the coding RNA is an mRNA.
E62. A nucleic acid according to E1, wherein the mRNA is not a replicon RNA or a self-replicating RNA.
E63. A nucleic acid according to any one of the preceding embodiments E59-E62, wherein the mRNA comprises at least one poly(A) sequence comprising 30 to 200 adenosine nucleotides and the 3′ terminal nucleotide is an adenosine.
E64. A nucleic acid according to any one of the preceding embodiments E58-E63, wherein the RNA, preferably the coding RNA, comprises a 5′-cap structure, preferably m7G, capO, cap1, cap2, a modified capO or a modified cap1 structure, preferably a 5′-cap1 structure.
E65. A nucleic acid according to any one of the preceding embodiments E58-E64, wherein the RNA is codon-optimized.
E66. A nucleic acid according to any one of the preceding embodiments E58-E65, wherein the RNA comprises a chemically modified nucleotide.
E67. A nucleic acid according to any one of the preceding embodiments E58-E66, wherein the RNA comprises 1-methylpseudouridine substitution. Preferably, all the uridines of the RNA are replaced by 1-methylpseudouridine.
E68. A nucleic acid according to any one of the preceding embodiments E58-E67, wherein the RNA is a purified RNA, preferably an RNA that has been purified by RP-HPLC and/or TFF.
E69. A nucleic according to any one of the preceding embodiments E58 to E68 wherein the RNA comprises the nucleic acid sequence of any of SEQ ID NO:411, SEQ ID NO:413, SEQ ID NO:415, SEQ ID NO:417 and SEQ ID NO:419, SEQ ID NO:504, SEQ ID NO:506, SEQ ID NO:508 and SEQ ID NO:510, SEQ ID NO: 295, SEQ ID NO: 297 and SEQ ID NO: 299.
E70. A composition comprising at least one nucleic acid according to any one of the preceding embodiments E53-E69.
E71. A composition comprising at least one nucleic acid according to any one of the preceding embodiments E53-E69, wherein the composition comprises at least one pharmaceutically acceptable carrier.
E72. A composition comprising at least one nucleic acid according to any one of the preceding embodiments E53-E69, wherein the composition is a multivalent composition comprising a plurality or at least more than one of the nucleic acid according to E53-E69.
E73. A composition comprising at least one nucleic acid according to any one of the preceding embodiments E53-E69, wherein the composition comprises RNA with an RNA integrity of 70% or more.
E74. A composition comprising at least one nucleic acid according to any one of the preceding embodiments E53-E69, wherein the composition comprises RNA with a capping degree of 70% or more, preferably wherein at least 70%, 80%, or 90% of the mRNA species comprise a Cap1 structure.
E75. A composition comprising at least one nucleic acid according to any one of the preceding embodiments E53-E69, wherein the at least one nucleic acid is complexed or associated with or at least partially complexed or partially associated with one or more cationic or polycationic compound, preferably cationic or polycationic polymer, cationic or polycationic polysaccharide, cationic or polycationic lipid, cationic or polycationic protein, cationic or polycationic peptide, or any combinations thereof.
E76. A composition comprising at least one nucleic acid according to any one of the preceding embodiments E53-E69, wherein the at least one nucleic acid is complexed or associated with one or more lipids or lipid-based carriers, thereby forming liposomes, lipid nanoparticles (LNP), lipoplexes, and/or nanoliposomes, preferably encapsulating the at least one nucleic acid.
E77. A composition comprising at least one nucleic acid according to any one of the preceding embodiments E53-E69, wherein the at least one nucleic acid is complexed with one or more lipids thereby forming lipid nanoparticles.
E78. A composition according to any one of the preceding embodiments E76-E77, wherein the LNP comprises a cationic lipid according to formula III-3:
E79.

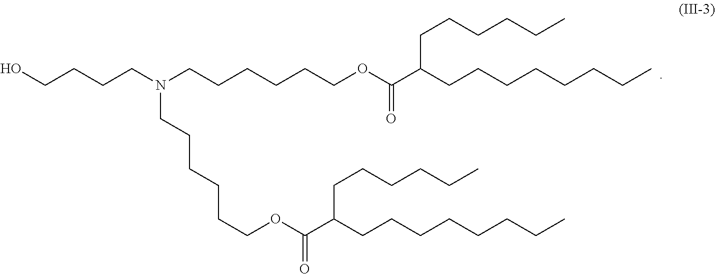 [SMILES: CCCCCCCCC(CCCCCC)C(=O)OCCCCCCN(CCCCO)CCCCCCOC(=O)C(CCCCCC)CCCCCCCC]

E80. A composition according to any one of the preceding embodiments E76-E78, wherein the LNP comprises a PEG lipid of formula (IVa):

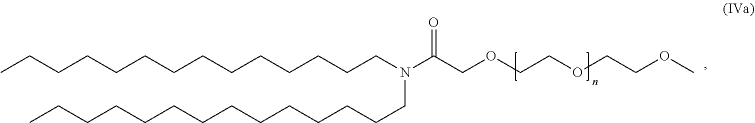

E81. A composition according to embodiment E79, wherein n has a mean value ranging from 30 to 60, preferably wherein n has a mean value of about 45, 46, 47, 48, 49, 50, 51, 52, 53, 54, most preferably wherein n has a mean value of 49 or 45.
E82. A composition according to any one of the preceding embodiments E76-E80, wherein the LNP comprises a PEG lipid of formula (IVa):

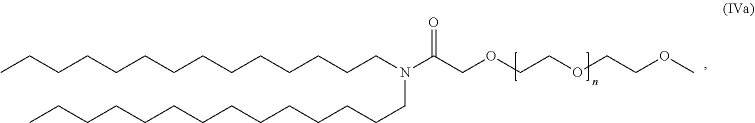

wherein n is an integer selected such that the average molecular weight of the PEG lipid is about 2500 g/mol.
E83. A composition according to any one of the preceding embodiments E76-E81, wherein the LNP comprises one or more neutral lipids and/or one or more steroid or steroid analogues.
E84. A composition according to any one of the preceding embodiments E76-E82, wherein the neutral lipid is 1,2-distearoyl-sn-glycero-3-phosphocholine (DSPC), preferably wherein the molar ratio of the cationic lipid to DSPC is in the range from about 2:1 to about 8:1.
E85. A composition according to any one of the preceding embodiments E76-E83, wherein the steroid is cholesterol, preferably wherein the molar ratio of the cationic lipid to cholesterol is in the range from about 2:1 to about 1:1.
E86. A composition according to any one of the preceding embodiments E76-E84, wherein the LNP comprises (i) at least one cationic lipid, preferably a lipid of formula (III), more preferably lipid 111-3; (ii) at least one neutral lipid, preferably 1,2-distearoyl-sn-glycero-3-phosphocholine (DSPC); (iii) at least one steroid or steroid analogue, preferably cholesterol; and (iv) at least one polymer conjugated lipid, preferably a PEG-lipid derived from formula (IVa, with n=49), wherein (i) to (iv) are in a molar ratio of about 20-60% cationic lipid, 5-25% neutral lipid, 25-55% sterol, and 0.5-15% PEG-lipid.
E87. A composition according to any one of the preceding embodiments E76-E85, wherein the LNP comprises (i) at least one cationic lipid, preferably a lipid of formula (III), more preferably lipid 111-3; (ii) at least one neutral lipid, preferably 1,2-distearoyl-sn-glycero-3-phosphocholine (DSPC); (iii) at least one steroid or steroid analogue, preferably cholesterol; and (iv) at least one polymer conjugated lipid, preferably a PEG-lipid derived from formula (IVa, with n=45), wherein (i) to (iv) are in a molar ratio of about 20-60% cationic lipid, 5-25% neutral lipid, 25-55% sterol, and 0.5-15% PEG-lipid.
E88. A composition according to any one of the preceding embodiments E76-E86, wherein (i) to (iv) are in a molar ratio of about 50:10:38.5:1.5, preferably 47.5:10:40.8:1.7 or more preferably 47.4:10:40.9:1.7.
E89. A composition according to any one of the preceding embodiments E76-E87, wherein the nucleic acid is RNA and the composition comprises less than about 20% free (non complexed or non-encapsulated) RNA, preferably less than about 15% free RNA, more preferably less than about 10% free RNA.
E90. A composition according to any one of the preceding embodiments E76-E88, wherein the wt/wt ratio of lipid to nucleic acid is from about 10:1 to about 60:1, preferably from about 20:1 to about 30:1, for example about 25:1.
E91. A composition according to any one of the preceding embodiments E76-E89, wherein the n/p ratio of the LNPs encapsulating the nucleic acid is in a range from about 1 to about 10, preferably in a range from about 5 to about 7, more preferably about 6.
E92. A composition according to any one of the preceding embodiments E76-E90, wherein the composition has a polydispersity index (PDI) value of less than about 0.4, preferably of less than about 0.3, more preferably of less than about 0.2, most preferably of less than about 0.1.
E93. A composition comprise according to any one of the preceding embodiments E76-E91, wherein the LNPs have a Z-average size in a range of about 60 nm to about 120 nm, preferably less than about 120 nm, more preferably less than about 100 nm, most preferably less than about 80 nm.
E94. A composition according to any one of the preceding embodiments E76-E92, wherein the LNPs comprise less than about 10%, 9%, 8%, 7%, 6%, 5%, 4%, 3%, 2%, 1% LNPs that have a particle size exceeding about 500 nm.
E95. A composition according to any one of the preceding embodiments E76-E93, wherein the LNPs comprise less than about 10%, 9%, 8%, 7%, 6%, 5%, 4%, 3%, 2%, 1% LNPs that have a particle size smaller than about 20 nm.
E96. A composition according to any one of the preceding embodiments E76-E94, wherein the LNP comprises (i) at least one cationic lipid; (ii) at least one neutral lipid; (iii) at least one steroid or steroid analogue; and (iv) at least one PEG-lipid, wherein (i) to (iv) are in a molar ratio of about 20-60% cationic lipid, 5-25% neutral lipid, 25-55% sterol, and 0.5-15% PEG-lipid.
E97. A composition according to any one of the preceding embodiments E76-E95, wherein the LNP comprises (i) at least one cationic lipid according to formula III-3; (ii) DSPC; (iii) cholesterol; and (iv) a PEG-lipid, according to formula IVa, wherein (i) to (iv) are in a molar ratio of about 20-60% cationic lipid, 5-25% neutral lipid, 25-55% sterol, and 0.5-15% PEG-lipid.
E98. A composition according to any one of the preceding embodiments E70-E96, wherein the composition is a lyophilized composition.
E99. An immunogenic composition comprising a mutant according to any one of E1 to E52, a nucleic acid according to any one of E53 to E69 or a composition according to E70 to E97.
E100. An immunogenic composition according to E98, further comprising a hMPV A antigen selected from the group consisting of a mutant of a wild-type hMPV A F protein and a nucleic acid encoding a mutant of a wild-type hMPV A F protein. In one embodiment, the hMPV A antigen is selected from mutants of a wild-type hMPV A F protein and a nucleic acids encoding a mutant of a wild-type hMPV A F protein disclosed in any of WO16103238, WO20234300, WO21222639, WO22076669, WO22214678, WO23102373, WO23110618, WO23217988 and WO23102388. In one embodiment, the hMPV A antigen is a mutant of a wild-type hMPV A F protein or a nucleic acid encoding a mutant of a wild-type hMPV A F protein comprising the mutations of mutant 115-BV as disclosed in Battles et al, Nature communication 8:1528 (2017).
E101. An immunogenic composition according to embodiment E99, wherein the hMPV A antigen is a mutant of a wild-type hMPV A F protein.
E102. An immunogenic composition according to embodiment E99, wherein the hMPV A antigen is a mutant of a wild-type hMPV A F protein from the present disclosure preferably from any of E1 to E72 of section B of the present disclosure.
E103. An immunogenic composition according to embodiment E99, wherein the hMPV A antigen comprises a nucleic acid encoding a mutant of a wild-type hMPV A F protein.
E104. An immunogenic composition according to embodiment E99, wherein the hMPV A antigen comprises a nucleic acid encoding a mutant of a wild-type hMPV A F protein from the present disclosure, preferably from any of E73 to E89 of section B of the present disclosure.
E105. An immunogenic composition according to any one of embodiments E98 to E103, further comprising a hMPV B antigen selected from the group consisting of a mutant of a wild-type hMPV B F protein and a nucleic acid encoding a mutant of a wild-type hMPV B F protein. In one embodiment, the hMPV B antigen is selected from mutants of a wild-type hMPV B F protein and a nucleic acids encoding a mutant of a wild-type hMPV B F protein disclosed in any of WO16103238, WO20234300, WO21222639, WO22076669, WO22214678, WO23102373, WO23110618, WO23217988 and WO23102388. In one embodiment, the hMPV B antigen is a mutant of a wild-type hMPV B F protein or a nucleic acid encoding a mutant of a wild-type hMPV B F protein comprising the mutations of mutant 115-BV as disclosed in Battles et al, Nature communication 8:1528 (2017).
E106. An immunogenic composition according to embodiment E104, wherein the hMPV B antigen is a mutant of a wild-type hMPV B F protein.
E107. An immunogenic composition according to embodiment E104, wherein the hMPV B antigen is a mutant of a wild-type hMPV B F protein from the present disclosure, preferably from any of E1 to E72 of section B of the present disclosure.
E108. An immunogenic composition according to embodiment E104, wherein the hMPV B antigen comprises a nucleic acid encoding a mutant of a wild-type hMPV B F protein.
E109. An immunogenic composition according to embodiment E104, wherein the hMPV B antigen comprises a nucleic acid encoding a mutant of a wild-type hMPV B F protein from the present disclosure, preferably from any of E73 to E89 of section B of the present disclosure.
E109. An immunogenic composition according to any one of embodiments E98 to E108, further comprising PIV1 antigen selected from the group consisting of a mutant of a wild-type PIV1 F protein and a nucleic acid encoding a mutant of a wild-type PIV1 F protein.
E110. An immunogenic composition according to embodiment E109, wherein the PIV1 antigen is a mutant of a wild-type PIV1 F protein.
E111. An immunogenic composition according to embodiment E109, wherein the PIV1 antigen is a mutant of a wild-type PIV1 F protein from the present disclosure, preferably from any of E1 to E56 of section C of the present disclosure.
E112. An immunogenic composition according to embodiment E109 wherein the PIV1 antigen comprises a nucleic acid encoding a mutant of a wild-type PIV1 F protein.
E113. An immunogenic composition according to embodiment E109, wherein the PIV1 antigen comprises a nucleic acid encoding a mutant of a wild-type PIV1 F protein from the present disclosure, preferably from any of E57 to E73 of section C of the present disclosure.
E114. An immunogenic composition according to embodiment E109, wherein the PIV1 antigen comprises a nucleic acid encoding a mutant of a wild-type PIV1 F protein as disclosed in WO2018081289 or WO2022207839.
E115. An immunogenic composition according to anyone of E98 to E114, further comprising an RSV antigen selected from the group consisting of a mutant of a wild-type RSV F protein of subtype A and a nucleic acid encoding a mutant of a wild-type RSV F protein of subtype A.
E116. An immunogenic composition according to embodiment E115, wherein the RSV antigen is a mutant of a wild-type RSV F protein of subtype A.
E117. An immunogenic composition according to embodiment E115, wherein the RSV antigen is a nucleic acid encoding a mutant of a wild-type RSV F protein of subtype A.
E118. An immunogenic composition according to embodiment E115, wherein the mutant of a wild-type RSV F protein of subtype A is disclosed in one of WO2009/079796, WO2010/149745, WO2011/008974, WO2014/160463, WO2014/174018, WO2014/202570, WO2015/013551, WO2015/177312, WO2017/005848, WO2017/174564, WO2017/005844, WO2017/109629, WO2022/002894 and WO2018/109220.
E119. An immunogenic composition according to any one of embodiments E98 to E118, further comprising an RSV antigen selected from the group consisting of a mutant of a wild-type RSV F protein of subtype B and a nucleic acid encoding a mutant of a wild-type RSV F protein of subtype B.
E120. An immunogenic composition according to embodiment E119, wherein the RSV antigen is a mutant of a wild-type RSV F protein of subtype B.
E121. An immunogenic composition according to embodiment E119, wherein the RSV antigen a nucleic acid encoding a mutant of a wild-type RSV F protein of subtype B.
E122. An immunogenic composition according to embodiment E119, wherein the mutant of a wild-type RSV F protein of subtype B is disclosed in one of WO2009/079796, WO2010/149745, WO2011/008974, WO2014/160463, WO2014/174018, WO2014/202570, WO2015/013551, WO2015/177312, WO2017/005848, WO2017/174564, WO2017/005844, WO2017/109629, WO2022/002894 and WO2018/109220.

### 2. PIV3 F PROTEIN MUTANTS

In some aspects, the present invention provides mutants of wild-type PIV3 F proteins, wherein the mutants display introduced mutations in the amino acid sequence relative to the amino acid sequence of the corresponding wild-type PIV3 F protein and are immunogenic against the wild-type PIV3 F protein in the prefusion conformation or against a virus comprising the wild-type F protein. In certain embodiments, the PIV3 F mutants possess certain beneficial characteristics, such as increased immunogenic properties or improved stability in the prefusion conformation of the mutants or prefusion trimeric conformation of the mutant, as compared to the corresponding wild-type F protein. In still other embodiments, the present disclosure provides PIV3 F mutants that display one or more introduced mutations as described herein and bind to a prefusion specific antibody selected from PIA174 mAb.

The introduced amino acid mutations in the PIV3 F protein mutants include amino acid substitutions, deletions, or additions. In some embodiments, the only mutations in the amino acid sequence of the mutants are amino acid substitutions relative to a wild-type PIV3 F protein.

The amino acid sequence of a large number of native PIV3 F proteins from different strains, as well as nucleic acid sequences encoding such proteins, is known in the art. For example, the sequence of several PIV3 F0 precursor proteins are set forth in SEQ ID NOs:300 to 304.

The native PIV3 F protein exhibits remarkable sequence conservation across different strains.

In view of the substantial conservation of PIV3 F protein sequences, a person of ordinary skill in the art can easily compare amino acid positions between different native PIV3 F protein sequences to identify corresponding PIV3 F protein amino acid positions between different PIV3 strains. For example, across nearly all identified native PIV3 F0 precursor proteins, the protease cleavage site falls in the same amino acid positions. Thus, the conservation of native PIV3 F protein sequences across strains and subtypes allows use of a reference PIV3 F sequence for comparison of amino acids at particular positions in the PIV3 F protein. For the purposes of this disclosure (unless context indicates otherwise), the PIV3 F protein amino acid positions are given with reference to the sequence of the F0 precursor polypeptide set forth in SEQ ID NO: 300 (the amino acid sequence of the full length native F precursor polypeptide of the PIV3 strain HPIV3/MEX/2545/2006; corresponding to Genbank Identifier AGT75285.1 (amino acids).

The consensus sequence for PIV3 (which correspond to SEQ ID NO: 300) was obtained as follows: Whole genome sequences for PIV3 were downloaded from NCBI's GenBank database as GenBank file format. Fusion protein gene sequences were filtered by sequence length to only include complete coding DNA sequence features. Translated fusion protein sequences were then parsed from GenBank file and saved as FASTA file. Muscle v5 was used to perform multiple sequence alignment of collected sequences. A Position specific score matrices (PSSMs) was generated to summarize the alignment information. For each column in the alignment, the number of each amino acid letters is counted and totaled. The consensus sequence at each position was calculated as the most common amino acid type in PSSM table. The final consensus sequence was then extracted and saved as FASTA file.

However, it should be noted, and one of skill in the art will understand, that different PIV3 F0 sequences may have different numbering systems, for example, if there are additional amino acid residues added or removed as compared to SEQ ID NO:300. As such, it is to be understood that when specific amino acid residues are referred to by their number, the description is not limited to only amino acids located at precisely that numbered position when counting from the beginning of a given amino acid sequence, but rather that the equivalent/corresponding amino acid residue in any and all PIV3 F sequences is intended even if that residue is not at the same precise numbered position, for example if the PIV3 sequence is shorter or longer than SEQ ID NO:300, or has insertions or deletions as compared to SEQ ID NO: 300.

### 2-1. Structure of the PIV3 F Protein Mutants

The PIV3 F protein mutants provided by the present disclosure comprise a F1 polypeptide and a F2 polypeptide. In several embodiments, the mutants further comprise a trimerization domain. In some embodiments, either the F1 polypeptide or the F2 polypeptide includes at least one introduced modification (e.g., amino acid substitution) as described in detail herein below. In some other embodiments, each of the F1 polypeptide and F2 polypeptide includes at least one introduced modification (e.g., amino acid substitution) as described in detail herein below.

2-1(a). F1 Polypeptide and F2 Polypeptide of the PIV3 F Mutants

The mature form of the PIV3 F protein comprises two separate polypeptide chains, namely the F1 polypeptide and F2 polypeptide bound by disulfide bonds. In some embodiments, the mutants of the disclosure are not cleaved and the F2 polypeptide and F1 polypeptide form a single polypeptide. The expression system (CHO cells) used for producing the mutants may not comprise the protease that would cleave the PIV3 F protein in a natural environment, thus would show limited cleavage.

The F1 polypeptide chain of the mutant may be of the same length as the full length F1 polypeptide of the corresponding wild-type PIV3 F protein; however, it may also have deletions, such as deletions of 1 up to 36 amino acid residues from the C-terminus of the full-length F1 polypeptide. A full-length F1 polypeptide of the PIV3 F mutants corresponds to amino acid positions 103-539 of the native PIV3 F0 precursor, and includes (from N- to C-terminus) an extracellular region (residues 103 to 493), a transmembrane domain (residues 494-514), and a cytoplasmic domain (residues 515-539). It should be noted that amino acid residues 481 onwards in a native F1 polypeptide sequence are optional sequences in a F1 polypeptide of the PIV3 F mutants provided herein, and therefore may be absent from the F1 polypeptide of the mutant.

In some embodiments, the F1 polypeptide of the PIV3 F mutants lacks the entire cytoplasmic domain. In other embodiments, the F1 polypeptide lacks the cytoplasmic domain and a portion of or all entire transmembrane domain. In some specific embodiments, the mutant comprises a F1 polypeptide wherein the amino acid residues from position 482 through 539 are absent. In some specific embodiments, the mutant comprises a F1 polypeptide wherein the amino acid residues from position 485 through 539 are absent. Typically, for mutants that are linked to trimerization domain, such as a foldon, amino acids 482 through 539 can be absent. Thus, in some specific embodiment, amino acid residues 482 through 539 are absent from the F1 polypeptide of the mutant. In still other specific embodiments, the F1 polypeptide of the PIV3 F mutants comprises or consists of amino acid residues 110-481 of a native F0 polypeptide sequence, such as any of the F0 precursor sequence set forth in SEQ ID Nos: 300 to 304.

On the other hand, the F1 polypeptide of the PIV3 F mutant may include a C-terminal linkage to a trimerization domain, such as a foldon. Many of the sequences of the PIV3 F mutants disclosed herein include a sequence of a PreScission cleavage site and Strep Tag II that are not essential for the function of the PIV3 F protein, such as for induction of an immune response. A person skilled in the art will recognize such sequences, and when appropriate, understand that these sequences are not included in a disclosed PIV3 F mutant.

In the PIV3 F mutants provided by the present disclosure, the F2 polypeptide chain may be of the same length as the full-length F2 polypeptide of the corresponding wild-type PIV3 F protein; it may also have deletions, such as deletions of 1, 2, 3, 4, 5, 6, 7, or 8 amino acid residues from the N-terminus or C-terminus of the F2 polypeptide.

The mutant in F0 form (i.e., a single chain polypeptide comprising the F2 polypeptide joined to the F1 polypeptide) or F1-F2 heterodimer form may form a protomer. The mutant may also be in the form of a trimer, which comprises three of the same protomer. Further, the mutants may be glycosylated proteins (i.e., glycoproteins) or non-glycosylated proteins. The mutant in F0 form may include, or may lack, the signal peptide sequence.

The F1 polypeptide and F2 polypeptide of the PIV3 F protein mutants to which one or more mutations are introduced can be from any wild-type PIV3 F proteins known in the art or discovered in the future, including, without limitations. In some embodiments, the PIV3 F mutant comprises a F1 and/or a F2 polypeptide from a PIV3 virus, from a known PIV3 F0 precursor protein such for example those set forth in any one of SEQ ID NOs: 300 to 304 to which one or more mutations are introduced.

In some embodiments, the PIV3 F protein mutants comprise a F1-polypeptide, a F2 polypeptide, and one or more introduced amino acid mutations as described herein below, wherein the F1 polypeptide comprises 350 consecutive amino acids and is at least 90, 95, 98, or 99 percent identical to amino acids 110-481 of any of the sequence of SEQ ID NO:300 to 304, wherein the F2 polypeptide comprises 70 consecutive amino acids and is at least 90, 95, 98, or 99 percent identical to amino acids 22-112 of any of the sequence of SEQ ID NO:300 to 304 and wherein PIV3 F protein mutant is stabilized in prefusion trimer conformation, whether as monomer or trimer.

2-1(b) Trimerization Domains

In several embodiments, the PIV3 F mutant provided by the present disclosure is linked to a trimerization domain. In some embodiments, the trimerization domain promotes the formation of trimer of three F1/F2 heterodimers.

Several exogenous trimerization domains that promote formation of stable trimers of soluble proteins are known in the art. Non limiting examples of such trimerization domains that can be linked to a mutant provided by the present disclosure include: (1) the GCN4 leucine zipper (Harbury et al. 1993 Science 262: 1401-1407); (2) the trimerization motif from the lung surfactant protein (Hoppe et al. 1994 FEB S Lett 344: 191-195); (3) collagen (McAlinden et al. 2003 Biol Chem 278:42200-42207); and (4) the phage T4 fibritin foldon (Miroshnikov et al. 1998 Protein Eng 11:329-414).

Typically, the trimerization domain is positioned C-terminal to the F1 polypeptide. It may join directly to the F1 polypeptide chain. Optionally, the multimerization domain is connected to the F1 polypeptide via a linker, such as an amino acid linker, for example the sequence GG, GS, GGGS, or SAIG. The linker can also be a longer linker (for example, including the repeat sequence GG). A preferred linker is GGGS. Numerous conformationally neutral linkers are known in the art that can be used in the mutants provided by the present disclosure. In some embodiments, the F mutant comprising a foldon domain include a protease cleavage site for removing the foldon domain from the F1 polypeptide, such as a thrombin site between the F1 polypeptide and the foldon domain.

In some embodiments, a foldon domain is linked to a F mutant at the C-terminus of F1 polypeptide. In specific embodiments, the foldon domain is a T4 fibritin foldon domain, such as the amino acid sequence GYIPEAPRDGQAYVRKDGEWVLLSTFL (SEQ ID NO: 7).

### 2-2. Introduced Mutations in the PIV3 F Protein Mutants

The PIV3 F mutants provided by the present disclosure comprise a F1 polypeptide and a F2 polypeptide, wherein (1) either the F1 polypeptide or (2) the F2 polypeptide, or (3) both the F1 polypeptide and F2 polypeptide include one or more introduced amino acid mutations relative to the amino acid sequence of the corresponding native F protein. The introduction of such amino acid mutations in the PIV3 F mutants confers a beneficial property to the mutants, such as enhanced immunogenicity, improved stability, improved expression or formation or improved stability of certain desired physical form or conformation of the mutants. Such introduced amino acid mutations are referred to as “engineered disulfide bond mutations,” “cavity filling mutations”, “proline substitution mutations” “cleavage site mutation” or “glycine replacement mutation” or electrostatic mutation”.

The nature and purpose of “engineered disulfide bond mutations”, “cavity filling mutations”, “proline substitution mutations” and “glycine replacement mutation” are already disclosed above in connection with hMPV protein mutants.

2-2(a) Engineered Disulfide Bond Mutations

In some embodiments, PIV3 protein F mutants provided by the present disclosure include one or more engineered disulfide bond mutations. The term “engineered disulfide bond mutation” refers to mutation of a pair of amino acid residues in a wild-type PIV3 F protein to a pair of cysteine residues. The introduced pair of cysteine residues allows for formation of a disulfide bond between the introduced cysteine residues, which disulfide bond serves to stabilize the protein's conformation or oligomeric state, such as prefusion conformation. For stabilizing the prefusion conformation of the mutant, the residue pairs for mutation to cysteine should be in close proximity in the prefusion conformation but distant in the post-fusion conformation. Preferably, the distance between the pair of residues (e.g. the beta carbons) is less than 8 Ain a prefusion conformation, but more than 20 Ain a post-fusion conformation.

In some embodiments, the PIV3 F protein mutants comprise only one engineered disulfide mutation (“single engineered disulfide mutation”). In some other embodiments, the PIV3 F protein mutants comprise at least two engineered disulfide mutations, wherein each pair of the cysteine residues of the engineered disulfide mutations are appropriately positioned when PIV3 F protein mutant is in prefusion conformation (“double engineered disulfide mutation”).

In some specific embodiments, the present disclosure provides a PIV3 F mutant comprising at least one engineered disulfide bond mutation, wherein the mutant comprises the same introduced mutations that are in any of the exemplary mutants provided in Tables 42 and 47.

The exemplary PIV3 F mutants provided in Tables 42 and 47 are based on the same F0 sequence of PIV3 of SEQ ID NO:305. The same introduced mutations in each of the mutants can be made to a native F0 polypeptide sequence of any other PIV3 subtype or strain to arrive at different PIV3 F mutants, such as a native F0 polypeptide sequence set forth in any of the SEQ ID NOs: 300-304 or from any other PIV3 strain. PIV3 F mutants that are based on a native F0 polypeptide sequence of any other PIV3 subtype or strain and comprise any of the engineered disulfide mutations are also within the scope of the invention. In some particular embodiments, a PIV3 F protein mutant comprises at least one engineered disulfide mutation such as 175C-202C, 160C-170C, 209C-234C, 209C-233C, 85C-209C and 162C-168C, preferably V175C-A202C, S160C-V170C, E209C-L234C, E209C-S233C, G85C-E209C and Q162C-L168C. In some particular embodiments, a PIV3 F protein mutant comprises at least one engineered disulfide mutation such as 160C-170C, preferably S160C-V170C.

2-2(b) Cavity Filling Mutations.

In other embodiments, the present disclosure provides PIV3 F mutants that comprise one or more cavity filling mutations. The term “cavity filling mutation” refers to the substitution of an amino acid residue in the wild-type PIV3 F protein by an amino acid that is expected to fill an internal cavity of the mature PIV3 F protein. In one application, such cavity-filling mutations contribute to stabilizing the prefusion conformation of a PIV3 F protein mutant. For example, the amino acids to be replaced for cavity-filling mutations typically include small aliphatic (e.g. Gly, Ala, and Val) or small polar amino acids (e.g. Ser and Thr). They may also include amino acids that are buried in the prefusion conformation, but exposed to solvent in the post-conformation. The replacement amino acids can aliphatic amino acids (Val, lie, Leu and Met), aromatic amino acid (His, Phe, Tyr and Trp), polar amino acids (Thr) with greater size than the replaced amino acids.

In some specific embodiments, a PIV3 F protein mutant comprises one or more cavity filling mutations at positions 463, 470, 474, and 477.

In some specific embodiments, the present disclosure provides a PIV3 F mutant comprising one or more cavity filling mutations, wherein the mutant comprises the cavity filling mutations in any of the mutants provided in Tables 44 and 47. PIV3 F mutants provided in Tables 44 and 47 are based on same native F0 sequence of PIV3 of SEQ ID NO:305. The same introduced mutations in each of the mutants can be made to a native F0 polypeptide sequence of any other PIV3 subtype or strain to arrive at different PIV3 F mutants, such as a native F0 polypeptide sequence set forth in any of the SEQ ID NOs: 300-304 or from any other PIV3 strain. The PIV3 F mutants that are based on a native F0 polypeptide sequence of any other PIV3 subtype or strain and comprise any of the one or more cavity filling mutations are also within the scope of the invention. In some particular embodiments, a PIV3 F protein mutant provided by the present disclosure comprises at least one cavity filling mutation selected from the group consisting of: S470A, S470L, S477A, A463L, 1474F and 1474Y.

### 2-2 (c) Proline Substitution Mutations.

In still other embodiments, the present disclosure provides PIV3 F protein mutants that include one or more proline substitution mutations. The term proline substitution mutations” refers to the substitution of an amine acid by a proline to prevent the structural refolding that occurs during transit from the prefusion to post-fusion conformation

In some specific embodiments, the PIV3 F protein mutant comprises the proline mutation S164P, G219P or S164P and G219P. In some specific embodiments, the present disclosure provides a PIV3 F mutant comprising one or more proline substitution mutations provided in Tables 43 and 44. PIV3 F mutant provided in Tables 43 and 44 is based on the native F0 sequence of PIV3 of SEQ ID NO:305. The same introduced mutation in the mutants can be made to a native F0 polypeptide sequence of any other PIV3 subtype or strain to arrive at different PIV3 F mutants, such as a native F0 polypeptide sequence set forth in any of the SEQ ID NOs:300-304 or from any other PIV3 strain. PIV3 F mutants that are based on a native F0 polypeptide sequence of any other PIV3 subtype or strain and comprise any of the one or more promine substitution mutations are also within the scope of the invention. In some particular embodiments, the PIV3 F protein mutant comprises mutation A128P.

### 2-2 (d) Glycine Replacement Mutations.

In still other embodiments, the present disclosure provides PIV3 F protein mutants that include one or more glycine replacement mutation. The term “glycine replacement mutation” refers to the replacement of a glycine by another amino acid in the middle of an α-helix to improve protein stability, preferably an amino acid without Cβ substitution, such as Ala, Leu or Met. In some specific embodiments, the present disclosure provides a PIV3 F mutant comprising one or more glycine replacement mutations, wherein the mutant comprises the glycine replacement mutation in the mutant provided in Table 44. PIV3 F mutants provided in Table 44 is based on the native F0 sequence of PIV3 of SEQ ID NO:305. The same introduced mutations in each of the mutants can be made to a native F0 polypeptide sequence of any other PIV3 subtype or strain to arrive at different PIV3 F mutants, such as a native F0 polypeptide sequence set forth in any of the SEQ ID NOs: 300-304 or from any other PIV3 strain. PIV3 F mutants that are based on a native F0 polypeptide sequence of any other PIV3 subtype or strain and comprise any of the one or more glycine replacement mutations are also within the scope of the invention. In some particular embodiments, the PIV3 F protein mutant comprises mutations G196A, G230A or G196A and G230A.

### 2-2 (e) Electrostatic Mutations

In still other embodiments, the present disclosure provides PIV3 F protein mutants that include one or more electrostatic mutations.

The term “electrostatic mutation” refers to an amino acid mutation introduced to a wild-type PIV 3 F protein that decreases ionic repulsion or increase ionic attraction between residues in a protein that are proximate to each other in the folded structure.

In some specific embodiments, the present disclosure provides a PIV3 F mutant comprising one or more electrostatic mutation, wherein the mutant comprises the electrostatic mutation in the mutant provided in Tables 44 and 45. PIV3 F mutants provided in Tables 44 and 45 is based on the native F0 sequence of PIV3 of SEQ ID NO:305. The same introduced mutations in each of the mutants can be made to a native F0 polypeptide sequence of any other PIV3 subtype or strain to arrive at different PIV3 F mutants, such as a native F0 polypeptide sequence set forth in any of the SEQ ID NOs: 300-304 or from any other PIV3 strain. PIV3 F mutants that are based on a native F0 polypeptide sequence of any other PIV3 subtype or strain and comprise any of the one or more electrostatic mutations are also within the scope of the invention. In some particular embodiments, the PIV3 F protein mutant comprises mutation E182L, D455S or E182L and D455S.

### 2-2 (f) Cleavage Site Mutation

The “cleavage site mutation” was introduced to prevent cleavage of the PIV3 F protein mutants between amino acids 109 and 110. However, it appeared that the PIV3 F protein mutants disclosed herein, when recombinantly expressed in CHO cells, were inefficiently cleaved between amino acids 109 and 110 even in the absence of any cleavage site mutation. As a result, the F1 and F2 polypeptides form a single polypeptide instead of two separate polypeptides linked by disulfide bonds. Unexpectedly, the “cleavage site mutation”, although not preventing cleavage which does not occur in the used expression system, provided some unexpected benefit in terms of thermal stability of the produced polypeptide.

In some specific embodiments, the present disclosure provides a PIV3 F mutant comprising one or more cleavage site mutations, wherein the mutant comprises the cleavage site mutation in the mutant provided in Table 46.

PIV3 F mutant provided in Tables 46 is based on the native F0 sequence of PIV3 of SEQ ID NO:305. The same introduced mutations in each of the mutants can be made to a native F0 polypeptide sequence of any other PIV3 subtype or strain to arrive at different PIV3 F mutants, such as a native F0 polypeptide sequence set forth in any of the SEQ ID NOs: 300-304 or from any other PIV3 strain. PIV3 F mutants that are based on a native F0 polypeptide sequence of any other PIV3 subtype or strain and comprise any of the one or more cleavage site mutations are also within the scope of the invention.

In some embodiment the cleavage site mutation comprises the following substitutions: R106G, T107S, E108A and R109S.

In some embodiment the cleavage site mutation comprises the following substitutions F110G and F111S.

PIV3 F mutants that include any additional mutations are also encompassed by the invention so long as the immunogenic property of the mutants is not substantially adversely affected by the additional mutations.

In some other particular embodiments, the present invention provides a PIV3 F mutant, wherein the mutant comprises an alanine at position 230, 470 and 477 (230AL, 470 Aand 477A) wherein the mutant comprises a F1 polypeptide and a F2 polypeptide selected from the group consisting of:
(1) a F2 polypeptide comprising the amino acid sequence of SEQ ID NO:329 and a F1 polypeptide comprising the amino acid sequence of SEQ ID NO:328;
(2) a F2 polypeptide comprising an amino acid sequence that is at least 97%, 98% or 99% identical to the amino acid sequence of SEQ ID NO:329 and a F1 polypeptide comprising an amino acid sequence that is at least 97%, 98%, or 99% identical to the amino acid sequence of SEQ ID NO:328.

In some other particular embodiments, the present invention provides a PIV3 F mutant, wherein the mutant comprises a cysteine at position 160 (160C) and 170 (170C), a leucine at position 463 (463L) and an alanine at position 230 (230A) wherein the mutant comprises a F1 polypeptide and a F2 polypeptide selected from the group consisting of:
(1) a F2 polypeptide comprising the amino acid sequence of SEQ ID NO:353 and a F1 polypeptide comprising the amino acid sequence of SEQ ID NO:352;
(2) a F2 polypeptide comprising an amino acid sequence that is at least 97%, 98% or 99% identical to the amino acid sequence of SEQ ID NO:353 and a F1 polypeptide comprising an amino acid sequence that is at least 97%, 98%, or 99% identical to the amino acid sequence of SEQ ID NO:352.

In some other particular embodiments, the present invention provides a PIV3 F mutant, wherein the mutant comprises a cysteine at position 160 (160C) and 170 (170C) and an alanine at position 470 (470A) and 477 (477A) wherein the mutant comprises a F1 polypeptide and a F2 polypeptide selected from the group consisting of:
(1) a F2 polypeptide comprising the amino acid sequence of SEQ ID NO:339 and a F1 polypeptide comprising the amino acid sequence of SEQ ID NO:338;
(2) a F2 polypeptide comprising an amino acid sequence that is at least 97%, 98% or 99% identical to the amino acid sequence of SEQ ID NO:339 and a F1 polypeptide comprising an amino acid sequence that is at least 97%, 98%, or 99% identical to the amino acid sequence of SEQ ID NO:338.

In some other particular embodiments, the present invention provides a PIV3 F mutant, wherein the mutant comprises a cysteine at position 160 (160C) and 170 (170C) and an alanine at position 230 (230A), 470 (470A) and 477 (477A) wherein the mutant comprises a F1 polypeptide and a F2 polypeptide selected from the group consisting of:
(1) a F2 polypeptide comprising the amino acid sequence of SEQ ID NO:351 and a F1 polypeptide comprising the amino acid sequence of SEQ ID NO:350;
(2) a F2 polypeptide comprising an amino acid sequence that is at least 97%, 98% or 99% identical to the amino acid sequence of SEQ ID NO:351 and a F1 polypeptide comprising an amino acid sequence that is at least 97%, 98%, or 99% identical to the amino acid sequence of SEQ ID NO:350.

In some other particular embodiments, the present invention provides a PIV3 F mutant, wherein the mutant comprises a cysteine at position 160 (160C) and 170 (170C), a leucine at position 463 (463L) and an alanine at position 230 (230A), 470 (470A) and 477 (477A) wherein the mutant comprises a F1 polypeptide and a F2 polypeptide selected from the group consisting of:
(1) a F2 polypeptide comprising the amino acid sequence of SEQ ID NO:355 and a F1 polypeptide comprising the amino acid sequence of SEQ ID NO:354;
(2) a F2 polypeptide comprising an amino acid sequence that is at least 97%, 98% or 99% identical to the amino acid sequence of SEQ ID NO:355 and a F1 polypeptide comprising an amino acid sequence that is at least 97%, 98%, or 99% identical to the amino acid sequence of SEQ ID NO:354.

The PIV3 F protein mutants provided by the present disclosure can be prepared by routine methods known in the art, such as by expression in a recombinant host system using a suitable vector. Suitable recombinant host cells include, for example, insect cells, mammalian cells, avian cells, bacteria, and yeast cells. Examples of suitable insect cells include, for example, Sf9 cells, Sf21 cells, Tn5 cells, Schneider S2 cells, and High Five cells (a clonal isolate derived from the parental Trichoplusia ni BTI-TN-5B1-4 cell line (Invitrogen)). Examples of suitable mammalian cells include Chinese hamster ovary (CHO) cells, human embryonic kidney cells (HEK293 or Expi293 cells, typically transformed by sheared adenovirus type 5 DNA), NIH-3T3 cells, 293-T cells, Vero cells, and HeLa cells. Suitable avian cells include, for example, chicken embryonic stem cells (e.g., EBx® cells), chicken embryonic fibroblasts, chicken embryonic germ cells, quail fibroblasts (e.g. ELL-O), and duck cells. Suitable insect cell expression systems, such as baculovirus-vectored systems, are known to those of skill in the art and described in, e.g., Summers and Smith, Texas Agricultural Experiment Station Bulletin No. 1555 (1987). Materials and methods for baculovirus/insect cell expression systems are commercially available in kit form from, inter alia, Invitrogen, San Diego Calif. Avian cell expression systems are also known to those of skill in the art and described in, e.g., U.S. Pat. Nos. 5,340,740; 5,656,479; 5,830,510; 6,114,168; and 6,500,668. Similarly, bacterial and mammalian cell expression systems are also known in the art and described in, e.g., Yeast Genetic Engineering (Barr et al., eds., 1989) Butterworths, London.

A number of suitable vectors for expression of recombinant proteins in insect or mammalian cells are well-known and conventional in the art. Suitable vectors can contain a number of components, including, but not limited to one or more of the following: an origin of replication; a selectable marker gene; one or more expression control elements, such as a transcriptional control element (e.g., a promoter, an enhancer, a terminator), and/or one or more translation signals; and a signal sequence or leader sequence for targeting to the secretory pathway in a selected host cell (e.g., of mammalian origin or from a heterologous mammalian or non-mammalian species). For example, for expression in insect cells a suitable baculovirus expression vector, such as pFastBac (Invitrogen), is used to produce recombinant baculovirus particles. The baculovirus particles are amplified and used to infect insect cells to express recombinant protein. For expression in mammalian cells, a vector that will drive expression of the construct in the desired mammalian host cell (e.g., Chinese hamster ovary cells) is used.

The PIV3 F protein mutant polypeptides can be purified using any suitable methods. For example, methods for purifying PIV3 F protein mutant polypeptides by immunoaffinity chromatography are known in the art. Ruiz-Arguello et al., J. Gen. Virol., 85:3677-3687 (2004). Suitable methods for purifying desired proteins including precipitation and various types of chromatography, such as hydrophobic interaction, ion exchange, affinity, chelating, and size exclusion are well-known in the art. Suitable purification schemes can be created using two or more of these or other suitable methods. If desired, the PIV3 F protein mutant polypeptides can include a “tag” that facilitates purification, such as an epitope tag, a strep II tag or a histidine (HIS) tag. Such tagged polypeptides can conveniently be purified, for example from conditioned media, by chelating chromatography or affinity chromatography.

TABLE 6
F protein sequences from selected PIV3 strains.

| | SEQ | |
|---|---|---|
| Strain/ | ID | |
| GenBank_aa | NO | F0 protein sequence |
| HPIV3/MEX/ | 300 | MLISILLIITTMIMASHCQIDITKLQHVGVLVNSPKGMKISQNFET |
| 2545/2006/ | | RYLILSLIPKIEDSNSCGDQQIKQYKRLLDRLIIPLYDGLRLQKDV |
| AGT75285.1 | | IVTNQESNENTDPRTERFFGGVIGTIALGVATSAQITAAVALVEA |
| | | KQARSDIEKLKEAIRDTNKAVQSVQSSVGNLIVAIKSVQDYVNK |
| | | EIVPSIARLGCEAAGLQLGIALTQHYSELTNIFGDNIGSLQEKGI |
| | | KLQGIASLYRTNITEIFTTSTVDKYDIYDLLFTESIKVRVIDVDLND |
| | | YSITLQVRLPLLTRLLNTQIYKVDSISYNIQNREWYIPLPSHIMTK |
| | | GAFLGGADVKECIEAFSSYICPSDPGFVLNHEMESCLSGNISQ |
| | | CPRTTVTSDIVPRYAFVNGGVVANCITTTCTCNGIGNRINQPPD |
| | | QGVKIITHKECNTIGINGMLFNTNKEGTLAFYTPDDITLNNSVAL |
| | | DPIDISIELNKAKSDLEESKEWIRRSNQKLDSIGSWHQSSTTIIVI |
| | | LIMMIILFIINITIITIAIKYYRIQKRNRVDQNDKPYVLTNK |
| HPIV3/ARG/ | 301 | MLASTLLIITTMIMASHCQIDITKLQHVGVLVNSPKGMKISQNFE |
| 10068/2004/ | | TRYLILSLIPKIEDSNSCGDQQIKQYKRLLDRLIIPLYDGLRLQKD |
| KF530245.1 | | VIVTNQESNENTDPRTERFFGGVIGTIALGVATSAQITAAVALVE |
| | | AKQARSDIEKLKEAIRDTNKAVQSVQSSVGNLIVAIKSVQDYVN |
| | | NEIVPSITRLGCEAAGLQLGIALTQHYSELTNIFGDNIGSLQEKG |
| | | IKLQGIASLYRTNITEIFTTSTVDKYDIYDLLFTESIKVRVIDVDLN |
| | | DYSITLQVRLPLLTRLLNTQIYKVDSISYNIQNREWYIPLPSHIMT |
| | | KGAFLGGADVKECIEAFSSYICPSDPGFVLNHEMESCLSGNIS |
| | | QCPRTTVTSDIVPRYAFVNGGWVANCITTTCTCNGIGNRINQPP |
| | | DQGVKIITHKECNTIGINGMLFNTNKEGTLAFYTPDDITLNNSVA |
| | | LDPIDISIELNKAKSDLEESKEWIRRSNQKLDSIGSWHQSSTTIIII |
| | | LIMMIILFIINVTIITIAIKYYRIQKRNRVDQNEKPYVLTNK |
| HPIV3/CHE/ | 302 | MLISILSIITTMIMASHCQIDITKLQHVGVLVNSPKGMKISQNFET |
| 1103040115/ | | RYLILSLIPKIEDSNSCGDQQIKQYKRLLDRLIIPLYDGLRLQKDV |
| 2011/ | | IVTNQESNENTDPRTERFFGGVIGTIALGVATSAQITAAVALVEA |
| KF530246.1 | | KQARSDIEKLKEAIRDTNKAVQSVQSSIGNLIVAIKSVQDYVNKE |
| | | IVPSIARLGCEAAGLQLGIALTQHYSELTNIFGDNIGSLQEKGIKL |
| | | QGIASLYRTNITEIFTTSTVDKYDIYDLLFTESIKVRVIDVDLNDY |
| | | SITLQVRLPLLTRLLNTQIYKVDSISYNIQNREWYIPLPSHIMTKG |
| | | AFLGGADVKECIEAFSSYICPSDPGFVLNHEMESCLSGNISQC |
| | | PRTTVTSDIVPRYAFVNGGVVANCITTTCTCNGIGNRINQPPDQ |
| | | GVKIITHKECNTIGINGMLFNTNKEGTLAFYTPDDITLNNSVALD |
| | | PIDISIELNKAKSDLEESKEWIRRSNQKLDSIGSWHQSSTTIIVILI |
| | | MMIILFIINITIITIAIKYYRIQKRNRVDQNDKPYVLTNK |
| HPIV3/Homo | 303 | MLISTLSIITTMIMASHCQIDITKLQHVGVLVNSPKGMKISQNFET |
| sapiens/PER/ | | RYLILSLIPKIEDSNSCGDQQIKQYKRLLDRLIIPLYDGLRLQKDV |
| FLA6024/2009/ | | IVTNQESNENTDPRTERFFGGVIGTIALGVATSAQITAAVALVEA |
| KJ672530.1 | | KQARSDIEKLKEAIRDTNKAVQSVQSSIGNLIVAIKSVQDYVNKE |
| | | IVPSIARLGCEAAGLQLGIALTQHYSELTNIFGDNIGSLQEKGIKL |
| | | QGIASLYRTNITEIFTTSTVDKYDIYDLLFTESIKVRVIDVDLNDY |
| | | SITLQVRLPLLTRLLNTQIYKVDSISYNIQNREWYIPLPSHVMTK |
| | | GAFLGGADVKECIEAFSSYICPSDPGFVLNHEMESCLSGNISQ |
| | | CPRTTVTSDIVPRYAFVNGGVVANCITTTCTCNGIGNRINQPPD |
| | | QGVKIITHKECNTIGINGMLFNTNKEGTLAFYTPDDITLNNSVAL |
| | | DPIDISIELNKAKSDLEESKKWIRRSNQKLDSIGSWHQSSTIIIIILI |
| | | MMIILFIINITIITIAIKYYRIQKRNRVDQNDKPYVLTNK |
| HPIV3/UK/395/ | 304 | MPISILLIITTMIMASHCQIDITKLQHVGVLVNSPKGMKISQNFET |
| 04/2015/ | | RYLILSLIPKIEDSNSCGDQQIKQYKRLLDRLIIPLYDGLRLQKDV |
| MH678693.1 | | IVTNQESNENTDPRTERFFGGVIGTIALGVATSAQITAAVALVEA |
| | | KQARSDIEKLKEAIRDTNKAVQSVQSSVGNLIVAIKSVQDYVNK |
| | | EIVPSIARLGCEAAGLQLGIALTQHYSELTNIFGDNIGSLQEKGI |
| | | KLQGIASLYRTNITEIFTTSTVDKYDIYDLLFTESIKVRVIDVDLND |
| | | YSITLQVRLPLLTRLLNTQIYKVDSISYNIQNREWYIPLPSHIMTK |
| | | GAFLGGADVKECIEAFSSYICPSDPGFILNHEMESCLSGNISQC |
| | | PRTTVTSDIVPRYAFVNGGVVANCITTTCTCNGIGNRINQPPDQ |
| | | GVKIITHKECNTIGINGMLFNTNKEGTLAFYTPDDITLNNSVALD |
| | | PIDISIELNKAKSDLEESKEWIRRSNQKLDSIGSWHQSSTTIIVILI |
| | | MMIILFIINITIITIAIKYYRIQKRNRMDQNDKPYVLTNK |

Table 7 provides the amino acid sequence of F1 polypeptide without transmembrane and intracellular domains and F2 polypeptide of variants of mutant PIV3110 (based on F protein sequence from HPIV3/MEX2545/2006 strain) to illustrate how a particular set of mutations applies to any PIV3 wild type F protein.

TABLE 7
Variants of Mutant PIV3110 and comprising introduced mutations G230A, S470A
and S477A

| | | | Amino Acid Sequence: |
|---|---|---|---|
| | Poly- | SEQ | (residues 110-481 for F1 polypeptide and |
| Mutant | peptide | ID | residues 19-109 for F2 polypeptide) |
| 110- | F1 | 376 | FFGGVIGTIALGVATSAQITAAVALVEAKQARSDIEKLKEA |
| HPIV3/MEX/ | | | IRDTNKAVQSVQSSVGNLIVAIKSVQDYVNKEIVPSIARLG |
| 2545/2006/ | | | CEAAGLQLGIALTQHYSELTNIFGDNIGSLQEKGIKLQAIA |
| AGT75285.1 | | | SLYRTNITEIFTTSTVDKYDIYDLLFTESIKVRVIDVDLNDY |
| | | | SITLQVRLPLLTRLLNTQIYKVDSISYNIQNREWYIPLPSHI |
| | | | MTKGAFLGGADVKECIEAFSSYICPSDPGFVLNHEMESC |
| | | | LSGNISQCPRTTVTSDIVPRYAFVNGGVVANCITTTCTCN |
| | | | GIGNRINQPPDQGVKIITHKECNTIGINGMLFNTNKEGTLA |
| | | | FYTPDDITLNNSVALDPIDISIELNKAKSDLEEAKEWIRRA |
| | | | NQKL |
| | F2 | 377 | QIDITKLQHVGVLVNSPKGMKISQNFETRYLILSLIPKIEDS |
| | | | NSCGDQQIKQYKRLLDRLIIPLYDGLRLQKDVIVTNQESN |
| | | | ENTDPRTER |
| 110- | F1 | 378 | FFGGVIGTIALGVATSAQITAAVALVEAKQARSDIEKLKEA |
| HPIV3/ARG/ | | | IRDTNKAVQSVQSSVGNLIVAIKSVQDYVNNEIVPSITRLG |
| 10068/2004/ | | | CEAAGLQLGIALTQHYSELTNIFGDNIGSLQEKGIKLQAIA |
| KF530245.1 | | | SLYRTNITEIFTTSTVDKYDIYDLLFTESIKVRVIDVDLNDY |
| | | | SITLQVRLPLLTRLLNTQIYKVDSISYNIQNREWYIPLPSHI |
| | | | MTKGAFLGGADVKECIEAFSSYICPSDPGFVLNHEMESC |
| | | | LSGNISQCPRTTVTSDIVPRYAFVNGGWVANCITTTCTCN |
| | | | GIGNRINQPPDQGVKIITHKECNTIGINGMLFNTNKEGTLA |
| | | | FYTPDDITLNNSVALDPIDISIELNKAKSDLEEAKEWIRRA |
| | | | NQKL |
| | F2 | 379 | QIDITKLQHVGVLVNSPKGMKISQNFETRYLILSLIPKIEDS |
| | | | NSCGDQQIKQYKRLLDRLIIPLYDGLRLQKDVIVTNQESN |
| | | | ENTDPRTER |
| 110- | F1 | 380 | FFGGVIGTIALGVATSAQITAAVALVEAKQARSDIEKLKEA |
| HPIV3/CHE/ | | | IRDTNKAVQSVQSSIGNLIVAIKSVQDYVNKEIVPSIARLG |
| 1103040115/ | | | CEAAGLQLGIALTQHYSELTNIFGDNIGSLQEKGIKLQAIA |
| 2011/ | | | SLYRTNITEIFTTSTVDKYDIYDLLFTESIKVRVIDVDLNDY |
| KF530246.1 | | | SITLQVRLPLLTRLLNTQIYKVDSISYNIQNREWYIPLPSHI |
| | | | MTKGAFLGGADVKECIEAFSSYICPSDPGFVLNHEMESC |
| | | | LSGNISQCPRTTVTSDIVPRYAFVNGGVVANCITTTCTCN |
| | | | GIGNRINQPPDQGVKIITHKECNTIGINGMLFNTNKEGTLA |
| | | | FYTPDDITLNNSVALDPIDISIELNKAKSDLEEAKEWIRRA |
| | | | NQKL |
| | F2 | 381 | QIDITKLQHVGVLVNSPKGMKISQNFETRYLILSLIPKIEDS |
| | | | NSCGDQQIKQYKRLLDRLIIPLYDGLRLQKDVIVTNQESN |
| | | | ENTDPRTER |
| 110- | F1 | 382 | FFGGVIGTIALGVATSAQITAAVALVEAKQARSDIEKLKEA |
| HPIV3/Homo | | | IRDTNKAVQSVQSSIGNLIVAIKSVQDYVNKEIVPSIARLG |
| sapiens/PER/ | | | CEAAGLQLGIALTQHYSELTNIFGDNIGSLQEKGIKLQAIA |
| FLA6024/ | | | SLYRTNITEIFTTSTVDKYDIYDLLFTESIKVRVIDVDLNDY |
| 2009/ | | | SITLQVRLPLLTRLLNTQIYKVDSISYNIQNREWYIPLPSHV |
| KJ672530.1 | | | MTKGAFLGGADVKECIEAFSSYICPSDPGFVLNHEMESC |
| | | | LSGNISQCPRTTVTSDIVPRYAFVNGGWVANCITTTCTCN |
| | | | GIGNRINQPPDQGVKIITHKECNTIGINGMLFNTNKEGTLA |
| | | | FYTPDDITLNNSVALDPIDISIELNKAKSDLEEAKKWIRRA |
| | | | NQKL |
| | F2 | 383 | QIDITKLQHVGVLVNSPKGMKISQNFETRYLILSLIPKIEDS |
| | | | NSCGDQQIKQYKRLLDRLIIPLYDGLRLQKDVIVTNQESN |
| | | | ENTDPRTER |
| 110- | F1 | 384 | FFGGVIGTIALGVATSAQITAAVALVEAKQARSDIEKLKEA |
| HPIV3/UK/ | | | IRDTNKAVQSVQSSVGNLIVAIKSVQDYVNKEIVPSIARLG |
| 395/04/2015/ | | | CEAAGLQLGIALTQHYSELTNIFGDNIGSLQEKGIKLQAIA |
| MH678693.1 | | | SLYRTNITEIFTTSTVDKYDIYDLLFTESIKVRVIDVDLNDY |
| | | | SITLQVRLPLLTRLLNTQIYKVDSISYNIQNREWYIPLPSHI |
| | | | MTKGAFLGGADVKECIEAFSSYICPSDPGFILNHEMESCL |
| | | | SGNISQCPRTTVTSDIVPRYAFVNGGVVANCITTTCTCN |
| | | | GIGNRINQPPDQGVKIITHKECNTIGINGMLFNTNKEGTLA |
| | | | FYTPDDITLNNSVALDPIDISIELNKAKSDLEEAKEWIRRA |
| | | | NQKL |
| | F2 | 385 | QIDITKLQHVGVLVNSPKGMKISQNFETRYLILSLIPKIEDS |
| | | | NSCGDQQIKQYKRLLDRLIIPLYDGLRLQKDVIVTNQESN |
| | | | ENTDPRTER |

### 3. Nucleic Acids Encoding PIV3 F Protein Mutants

In another aspect, the present invention provides nucleic acid molecules that encode a PIV3 F protein mutant described herein above. These nucleic acid molecules include DNA, cDNA, and RNA sequences. Nucleic acid molecules that encode only a F2 polypeptide or only a F1 polypeptide of a PIV3 F mutant are also encompassed by the invention. The nucleic acid molecule can be incorporated into a vector, such as an expression vector.

In some embodiments, the nucleic acid molecule encodes a precursor F0 polypeptide that, when expressed in an appropriate cell, is processed into a disclosed PIV3 F mutant. In some embodiments, the nucleic acid molecule encodes a precursor F0 polypeptide that, when expressed in an appropriate cell, is processed into a disclosed PIV3 F mutant, wherein the precursor F0 polypeptide includes, from N- to C-terminus, a signal peptide, a F2 polypeptide, and a F1 polypeptide. In some embodiments, the signal peptide comprises the amino acid sequence set forth as positions 1-21 of any one SEQ ID NOs: 300 to 304, wherein the amino acid positions correspond to the amino acid sequence of a reference of SEQ ID NO:300.

In a preferred embodiment, the nucleic acid is an RNA, more preferably an mRNA. In a preferred embodiment, the mRNA encodes a precursor F0 polypeptide that, when expressed in an appropriate cell, is processed into a full length PIV3 F protein mutant disclosed herein (i.e comprising one or more mutations, a full length F1 polypeptide and a full length F2 polypeptide). A full-length F1 polypeptide of the PIV3 F mutants corresponds to amino acid positions 103-539 of the native PIV3 F0 precursor, and includes (from N- to C-terminus) an extracellular region (residues 103 to 493), a transmembrane domain (residues 494-514), and a cytoplasmic domain (residues 515-539). In a preferred embodiment, the nucleic acid is an mRNA comprising a chemically modified nucleotide. In a preferred embodiment, the nucleic acid is an mRNA comprising a chemically modified nucleotide, preferably 1-methylpseudouridine. Preferably, all the uridines of the RNA are replaced by 1-methylpseudouridine.

In some specific embodiments, the present disclosure provides a nucleic acid molecule which encodes a mutant comprising the mutations selected from the group consisting of:
(1) V175C and A202C;
(2) S160C and V170C;
(3) S164P;
(4) G196A;
(5) G219P;
(6) G230A;
(7) E182L;
(8) S470A;
(9) S477A;
(10) S470A and S477A;
(11) D455S;
(12) A463L;
(13) Q162C, L168C, S470A and S477A;
(14) S160C, V170C, S470A and S477A;
(15) G230A, S470A and S477A;
(16) A463L, S470A and S477A;
(17) E209C and L234C,
(18) A463L and S470L,
(19) S160C, V170C, E209C-L234C, A463L and S470L;
(20) S160C, V170C, E209C, L234C, A463L and 1474F;
(21) S160C, V170C, E209C, L234C, A463L, S470L, F110G, F111S;
(22) S160C, V170C, A463L and S470L;
(23) Q162C, L168C, G230A, A463V and 1474Y;
(24) Q162C, L168C, G230A, S470A and S477A;
(25) Q162C, L168C, G230A and A463L;
(26) Q162C, L168C, G230A, A463L, S470A and S477A;
(27) S160C, V170C, G230A, A463V and 1474Y;
(28) S160C, V170C, G230A, S470A and S477A;
(29) S160C, V170C, G230A and A463L;
(30) S160C, V170C, G230A, A463L, S470A and S477A; and
(31) S160C, V170C and A463L.

In some specific embodiments, the present disclosure provides a nucleic acid molecule, preferably a mRNA, more preferably a mRNA wherein all the uridines are replaced by 1-methylpseudouridine, said nucleic acid encoding a precursor F0 polypeptide that, when expressed in an appropriate cell, is processed into a full length PIV3 F protein mutant disclosed herein comprising the mutations selected from the group consisting of
(1) V175C and A202C;
(2) S160C and V170C;
(3) S164P;
(4) G196A;
(5) G219P;
(6) G230A;
(7) E182L;
(8) S470A;
(9) S477A;
(10) S470A and S477A;
(11) D455S;
(12) A463L;
(13) Q162C, L168C, S470A and S477A;
(14) S160C, V170C, S470A and S477A;
(15) G230A, S470A and S477A;
(16) A463L, S470A and S477A;
(17) E209C and L234C;
(18) A463L and S470L;
(19) S160C, V170C, E209C, L234C, A463L and S470L;
(20) S160C, V170C, E209C, L234C, A463L and 1474F;
(21) S160C, V170C, E209C, L234C, A463L, S470L, F110G, F111S;
(22) S160C, V170C, A463L and S470L;
(23) Q162C, L168C, G230A, A463V and 1474Y;
(24) Q162C, L168C, G230A, S470A and S477A;
(25) Q162C, L168C, G230A and A463L;
(26) Q162C, L168C, G230A, A463L, S470A and S477A;
(27) S160C, V170C, G230A, A463V and 1474Y;
(28) S160C, V170C, G230A, S470A and S477A;
(29) S160C, V170C, G230A and A463L;
(30) S160C, V170C, G230A, A463L, S470A and S477A;
(31) S160C, V170C and A463L;
(32) E209C and S233C;
(33) G85C and E209C;
(34) T277V;
(35) A463L and 1474F;
(36) A4631, S470I (37) S160C, V170C, E209C, S233C, A463L and S470L;
(38) S160C, V170C, E209C, S233C, A463L and 1474F;
(39) S160C, V170C, G85C, E209C, A463L and S470L;
(40) S160C, V170C, G85C, E209C, A463L and 1474F;
(41) S160C, V170C, E209C, L234C, T277V, A463L and S470L;
(42) S160C, V170C, E209C, L234C, T277V, A463L and 1474F;
(43) S160C, V170C, E209C, S233C, T277V, A463L and S470L;
(44) S160C, V170C, E209C, S233C, T277V, A463L and 1474F;
(45) S160C, V170C, G85C, E209C, T277V, A463L and 1474F;
(46) S160C, V170C, E209C, L234C, D455S, A463L and S470L;
(47) S160C, V170C, E209C, S233C, D455S, A463L and S470L;
(48) S160C, V170C, G85C, E209C, D455S, A463L and S470L;
(49) S160C, V170C, E209C, L234C, T277V, D455S, A463L and S470L;
(50) S160C, V170C, E209C, S233C, T277V, D455S, A463L and S470L;
(51) S160C, V170C, G85C, E209C, T277V, D455S, A463L and S470L;
(52) S160C, V170C and S470L;
(53) R106G, T107S, E108A, R109S, S160C, V170C, E209C, L234C, A463L and S470L;
(54) R106G, T107S, E108A, R109S, S160C, V170C, E209C, S233C, A463L and S470L;
(55) R106G, T107S, E108A, R109S, S160C, V170C, G85C, E209C, A463L and S470L;
(56) F110G, F111S, S160C, V170C, E209C, L234C, A463L and S470L;
(57) F110G, F111S, S160C, V170C, E209C, S233C, A463L and S470L;
(58) F110G, F111S, S160C, V170C, A463L and S470L;
(59) F110G, F111S, S160C, V170C and S470L;
(60) S160C, V170C, A463L and S477L;
(61) S160C, V170C, E209C, L234C, A463L and S470L; and,
(62) S160C, V170C and S470L.

In some specific embodiments, the present disclosure provides a nucleic acid molecule, preferably a mRNA, more preferably a mRNA wherein all the uridines are replaced by 1-methylpseudouridine, said nucleic acid encoding a precursor F0 polypeptide that, when expressed in an appropriate cell, is processed into a full length PIV3 F protein mutant disclosed herein comprising the mutations selected from the group consisting of
(1) G230A, S470A and S477A;
(2) S160C, V170C, G230A and A463L;
(3) S160C, V170C, S470A and S477A;
(4) S160C, V170C, G230A, S470A and S477A;
(5) S160C, V170C, G230A, A463L, S470A and S477A;
(6) S160C, V170C, E209C, L234C, A463L and S470L;
(7) S160C, V170C, E209C, L234C, A463L and 1474F;
(8) S160C, V170C, E209C, L234C, A463L, S470L, F110G, F111S; and,
(9) S160C, V170C, A463L and S470L.
   E. Immunogenic Compositions Comprising a hMPV A, hMPV B, PIV1 and/or PIV3 F Protein Mutant and/or a Nucleic Acid Encoding a hMPV A, hMPV B, PIV1 or PIV3 F Protein Mutant

In another aspect, the invention provides immunogenic compositions that comprise (1) a hMPV A, hMPV B, PIV1 or PIV3 F protein mutant described in the disclosure, and/or (2) a nucleic acid molecule, preferably modRNA, or vector encoding such a hMPV A, hMPV B, PIV1 or PIV3 F protein mutant.

In one embodiment, the term modRNA, as used in this section, preferably refers to an mRNA encoding a precursor F0 polypeptide that, when expressed in an appropriate cell, is processed into a full length F protein mutant disclosed herein (i.e comprising one or more mutations, a full length polypeptide and a full length F2 polypeptide), preferably wherein all the uridines of the RNA are replaced by 1-methylpseudouridine.

In some embodiments, the immunogenic composition comprise one, two, three or four mutants selected from the group consisting of:
(1) a hMPV A F protein mutant or a nucleic acid, preferably a modRNA, encoding a protein mutant described in the disclosure;
(2) a hMPV B F protein mutant or a nucleic acid, preferably a modRNA encoding a protein mutant described in the disclosure;
(3) a PIV1 F protein mutant or a nucleic acid, preferably a modRNA encoding a protein mutant described in the disclosure, and,
(4) a PIV3 F protein mutant or a nucleic acid, preferably a modRNA encoding a protein mutant described in the disclosure.

In some embodiments, the immunogenic composition comprises a hMPV A F protein mutant or a nucleic acid, preferably a modRNA, encoding a hMPV A F protein mutant described in the disclosure.

In some embodiments, the immunogenic composition comprises a hMPV A F protein mutant or a nucleic acid, preferably a modRNA, encoding a hMPV A F protein mutant described in the disclosure and a hMPV B antigen.

In some embodiments, the immunogenic composition comprises a hMPV A F protein mutant or a nucleic acid, preferably a modRNA, encoding a hMPV A F protein mutant described in the disclosure and a hMPV B F protein mutant or a nucleic acid, preferably a modRNA, encoding a hMPV B F protein mutant described in the disclosure.

In some embodiments, the immunogenic composition comprises a hMPV A F protein mutant or a nucleic acid, preferably a modRNA, encoding a hMPV A F protein mutant described in the disclosure and a PIV1 antigen.

In some embodiments, the immunogenic composition comprises a hMPV A F protein mutant or a nucleic acid, preferably a modRNA, encoding a hMPV A F protein mutant described in the disclosure and a PIV1 F protein mutant or a nucleic acid, preferably a modRNA, encoding a PIV1 F protein mutant described in the disclosure.

In some embodiments, the immunogenic composition comprises a hMPV A F protein mutant or a nucleic acid, preferably a modRNA, encoding a hMPV A F protein mutant described in the disclosure and a PIV3 antigen.

In some embodiments, the immunogenic composition comprises a hMPV A F protein mutant or a nucleic acid, preferably a modRNA, encoding a hMPV A F protein mutant described in the disclosure and a PIV3 F protein mutant or a nucleic acid, preferably a modRNA, encoding a PIV3 F protein mutant described in the disclosure or a PIV3 F protein mutant or a nucleic acid, preferably a modRNA, encoding a PIV3 F protein mutant disclosed in WO2018081289 or WO22207839.

In some embodiments, the immunogenic composition comprises a hMPV A F protein mutant or a nucleic acid, preferably a modRNA, encoding a hMPV A F protein mutant described in the disclosure, a PIV1 antigen and a PIV3 antigen.

In some embodiments, the immunogenic composition comprises a hMPV A F protein mutant or a nucleic acid, preferably a modRNA, encoding a hMPV A F protein mutant described in the disclosure, a PIV1 protein mutant or a nucleic acid, preferably a modRNA, encoding a PIV1 F protein mutant described in the disclosure and a PIV3 protein mutant or a nucleic acid, preferably a modRNA, encoding a PIV3 F protein mutant described in the disclosure or disclosed in WO2018081289 or WO22207839.

In some embodiments, the immunogenic composition comprises a hMPV A F protein mutant or a nucleic acid, preferably a modRNA, encoding a hMPV A F protein mutant described in the disclosure, a hMPV B antigen antigen and a PIV3 antigen.

In some embodiments, the immunogenic composition comprises a hMPV A F protein mutant or a nucleic acid, preferably a modRNA, encoding a hMPV A F protein mutant described in the disclosure, a hMPV B F protein mutant or a nucleic acid, preferably a modRNA, encoding a hMPV B F protein mutant described in the disclosure and a PIV3 protein mutant or a nucleic acid, preferably a modRNA, encoding a PIV3 F protein mutant described in the disclosure or disclosed in WO2018081289 or WO22207839.

In some embodiments, the immunogenic composition comprises a hMPV A F protein mutant or a nucleic acid, preferably a modRNA, encoding a hMPV A F protein mutant described in the disclosure, a hMPV B antigen antigen and a PIV1 antigen.

In some embodiments, the immunogenic composition comprises a hMPV A F protein mutant or a nucleic acid, preferably a modRNA, encoding a hMPV A F protein mutant described in the disclosure, a hMPV B F protein mutant or a nucleic acid, preferably a modRNA, encoding a hMPV B F protein mutant described in the disclosure and a PIV1 protein mutant or a nucleic acid, preferably a modRNA, encoding a PIV1 F protein mutant described in the disclosure.

In some embodiments, the immunogenic composition comprises a hMPV A F protein mutant or a nucleic acid, preferably a modRNA, encoding a hMPV A F protein mutant described in the disclosure, a hMPV B antigen, a PIV1 antigen and a PIV3 antigen.

In some embodiments, the immunogenic composition comprises a hMPV A F protein mutant or a nucleic acid, preferably a modRNA, encoding a hMPV A F protein mutant described in the disclosure, a hMPV B F protein mutant or a nucleic acid, preferably a modRNA, encoding a hMPV B F protein mutant described in the disclosure, a PIV3 F protein mutant or a nucleic acid, preferably a modRNA, encoding a PIV3 F protein mutant described in the disclosure or disclosed in WO2018081289 or WO22207839 and a PIV1 protein mutant or a nucleic acid, preferably a modRNA, encoding a PIV1 F protein mutant described in the disclosure.

In some embodiments, the immunogenic composition comprises a hMPV B F protein mutant or a nucleic acid, preferably a modRNA, encoding a hMPV B F protein mutant described in the disclosure.

In some embodiments, the immunogenic composition comprises a hMPV B F protein mutant or a nucleic acid, preferably a modRNA, encoding a hMPV B F protein mutant described in the disclosure and a hMPV A antigen.

In some embodiments, the immunogenic composition comprises a hMPV B F protein mutant or a nucleic acid, preferably a modRNA, encoding a hMPV B F protein mutant described in the disclosure and a hMPV A F protein mutant or a nucleic acid, preferably a modRNA, encoding a hMPV A F protein mutant described in the disclosure.

In some embodiments, the immunogenic composition comprises a hMPV B F protein mutant or a nucleic acid, preferably a modRNA, encoding a hMPV B F protein mutant described in the disclosure and a PIV1 antigen.

In some embodiments, the immunogenic composition comprises a hMPV B F protein mutant or a nucleic acid, preferably a modRNA, encoding a hMPV B F protein mutant described in the disclosure and a PIV1 F protein mutant or a nucleic acid, preferably a modRNA, encoding a PIV1 F protein mutant described in the disclosure.

In some embodiments, the immunogenic composition comprises a hMPV B F protein mutant or a nucleic acid, preferably a modRNA, encoding a hMPV B F protein mutant described in the disclosure and a PIV3 antigen.

In some embodiments, the immunogenic composition comprises a hMPV B F protein mutant or a nucleic acid, preferably a modRNA, encoding a hMPV B F protein mutant described in the disclosure and a PIV3 F protein mutant or a nucleic acid, preferably a modRNA, encoding a PIV3 F protein mutant described in the disclosure or a PIV3 F protein mutant or a nucleic acid, preferably a modRNA, encoding a PIV3 F protein mutant disclosed in WO2018081289 or WO22207839.

In some embodiments, the immunogenic composition comprises a hMPV B F protein mutant or a nucleic acid, preferably a modRNA, encoding a hMPV B F protein mutant described in the disclosure, a PIV1 antigen and a PIV3 antigen.

In some embodiments, the immunogenic composition comprises a hMPV B F protein mutant or a nucleic acid, preferably a modRNA, encoding a hMPV B F protein mutant described in the disclosure, a PIV1 protein mutant or a nucleic acid, preferably a modRNA, encoding a PIV1 F protein mutant described in the disclosure and a PIV3 protein mutant or a nucleic acid, preferably a modRNA, encoding a PIV3 F protein mutant described in the disclosure or disclosed in WO2018081289 or WO22207839.

In some embodiments, the immunogenic composition comprises a hMPV B F protein mutant or a nucleic acid, preferably a modRNA, encoding a hMPV B F protein mutant described in the disclosure, a hMPV A antigen antigen and a PIV3 antigen.

In some embodiments, the immunogenic composition comprises a hMPV B F protein mutant or a nucleic acid, preferably a modRNA, encoding a hMPV B F protein mutant described in the disclosure, a hMPV A F protein mutant or a nucleic acid, preferably a modRNA, encoding a hMPV A F protein mutant described in the disclosure and a PIV3 protein mutant or a nucleic acid, preferably a modRNA, encoding a PIV3 F protein mutant described in the disclosure or disclosed in WO2018081289 or WO22207839.

In some embodiments, the immunogenic composition comprises a hMPV B F protein mutant or a nucleic acid, preferably a modRNA, encoding a hMPV B F protein mutant described in the disclosure, a hMPV A antigen antigen and a PIV1 antigen.

In some embodiments, the immunogenic composition comprises a hMPV B F protein mutant or a nucleic acid, preferably a modRNA, encoding a hMPV B F protein mutant described in the disclosure, a hMPV A F protein mutant or a nucleic acid, preferably a modRNA, encoding a hMPV A F protein mutant described in the disclosure and a PIV1 protein mutant or a nucleic acid, preferably a modRNA, encoding a PIV1 F protein mutant described in the disclosure.

In some embodiments, the immunogenic composition comprises a hMPV B F protein mutant or a nucleic acid, preferably a modRNA, encoding a hMPV B F protein mutant described in the disclosure, a hMPV A antigen, a PIV1 antigen and a PIV3 antigen.

In some embodiments, the immunogenic composition comprises hMPV B F protein mutant or a nucleic acid, preferably a modRNA, encoding a hMPV B F protein mutant described in the disclosure, a hMPV A F protein mutant or a nucleic acid, preferably a modRNA, encoding a hMPV A F protein mutant described in the disclosure, a PIV3 protein mutant or a nucleic acid, preferably a modRNA, encoding a PIV3 F protein mutant described in the disclosure or disclosed in WO2018081289 or WO22207839 and a PIV1 protein or protein mutant or a nucleic acid, preferably a modRNA, encoding a PIV1 F protein mutant described in the disclosure.

In some embodiments, the immunogenic composition comprises a PIV1 F protein mutant or a nucleic acid, preferably a modRNA, encoding a PIV1 F protein mutant described in the disclosure.

In some embodiments, the immunogenic composition comprises a PIV1 F protein mutant or a nucleic acid, preferably a modRNA, encoding a PIV1 F protein mutant mutant described in the disclosure and a hMPV A antigen. In some embodiments, the hMPV A antigen is selected from mutants of a wild-type hMPV A F protein and a nucleic acids encoding a mutant of a wild-type hMPV A F protein disclosed in any of WO16103238, WO20234300, WO21222639, WO22076669, WO22214678, WO23102373, WO23110618, WO23217988 and WO23102388. In some embodiments, the hMPV A antigen is a mutant of a wild-type hMPV A F protein or a nucleic acid encoding a mutant of a wild-type hMPV A F protein comprising the mutations of mutant 115-BV as disclosed in Battles et al, Nature communication 8:1528 (2017).

In some embodiments, the immunogenic composition comprises a PIV1 F protein mutant or a nucleic acid, preferably a modRNA, encoding a PIV1 F protein mutant mutant described in the disclosure and a hMPV A F protein mutant or a nucleic acid, preferably a modRNA, encoding a hMPV A protein mutant mutant described in the disclosure.

In some embodiments, the immunogenic composition comprises a PIV1 F protein mutant or a nucleic acid, preferably a modRNA, encoding a PIV1 F protein mutant described in the disclosure and a hMPV B antigen. In some embodiments, the hMPV B antigen is selected from mutants of a wild-type hMPV B F protein and a nucleic acids encoding a mutant of a wild-type hMPV B F protein disclosed in any of WO16103238, WO20234300, WO21222639, WO22076669, WO22214678, WO23102373, WO23110618, WO23217988 and WO23102388. In some embodiments, the hMPV B antigen is a mutant of a wild-type hMPV B F protein or a nucleic acid encoding a mutant of a wild-type hMPV B F protein comprising the mutations of mutant 115-BV as disclosed in Battles et al, Nature communication 8:1528 (2017).

In some embodiments, the immunogenic composition comprises a PIV1 F protein mutant or a nucleic acid, preferably a modRNA, encoding a PIV1 F protein mutant described in the disclosure and a hMPV B F protein mutant or a nucleic acid, preferably a modRNA, encoding a hMPV B F protein mutant described in the disclosure.

In some embodiments, the immunogenic composition comprises a PIV1 F protein mutant or a nucleic acid, preferably a modRNA, encoding a PIV1 F protein mutant described in the disclosure and a PIV3 antigen.

In some embodiments, the immunogenic composition comprises a PIV1 F protein mutant or a nucleic acid, preferably a modRNA, encoding a PIV1 F protein mutant described in the disclosure and a PIV3 F protein mutant or a nucleic acid, preferably a modRNA, encoding a PIV3 F protein mutant described in the disclosure or a PIV3 F protein mutant or a nucleic acid, preferably a modRNA, encoding a PIV3 F protein mutant disclosed in WO2018081289 or WO22207839.

In some embodiments, the immunogenic composition comprises a PIV1 F protein mutant or a nucleic acid, preferably a modRNA, encoding a PIV1 F protein mutant described in the disclosure, a hMPV B antigen and a PIV3 antigen. In some embodiments, the hMPV B antigen is selected from mutants of a wild-type hMPV B F protein and a nucleic acids encoding a mutant of a wild-type hMPV B F protein disclosed in any of WO16103238, WO20234300, WO21222639, WO22076669, WO22214678, WO23102373, WO23110618, WO23217988 and WO23102388. In some embodiments, the hMPV B antigen is a mutant of a wild-type hMPV B F protein or a nucleic acid encoding a mutant of a wild-type hMPV B F protein comprising the mutations of mutant 115-BV as disclosed in Battles et al, Nature communication 8:1528 (2017).

In some embodiments, the immunogenic composition comprises a PIV1 F protein mutant or a nucleic acid, preferably a modRNA, encoding a PIV1 F protein mutant described in the disclosure, a hMPV B protein mutant or a nucleic acid, preferably a modRNA, encoding a hMPV B F protein mutant described in the disclosure and a PIV3 protein mutant or a nucleic acid, preferably a modRNA, encoding a PIV3 F protein mutant described in the disclosure or disclosed in WO2018081289 or WO22207839.

In some embodiments, the immunogenic composition comprises a PIV1 F protein mutant or a nucleic acid, preferably a modRNA, encoding a PIV1 F protein mutant described in the disclosure, a hMPV A antigen and a PIV3 antigen. In some embodiments, the hMPV A antigen is selected from mutants of a wild-type hMPV A F protein and a nucleic acids encoding a mutant of a wild-type hMPV A F protein disclosed in any of WO16103238, WO20234300, WO21222639, WO22076669, WO22214678, WO23102373, WO23110618, WO23217988 and WO23102388. In some embodiments, the hMPV A antigen is a mutant of a wild-type hMPV A F protein or a nucleic acid encoding a mutant of a wild-type hMPV A F protein comprising the mutations of mutant 115-BV as disclosed in Battles et al, Nature communication 8:1528 (2017).

In some embodiments, the immunogenic composition comprises a PIV1 F protein mutant or a nucleic acid, preferably a modRNA, encoding a PIV1 F protein mutant described in the disclosure, a hMPV A F protein mutant or a nucleic acid, preferably a modRNA, encoding a hMPV A F protein mutant described in the disclosure and a PIV3 protein mutant or a nucleic acid, preferably a modRNA, encoding a PIV3 F protein mutant described in the disclosure or disclosed in WO2018081289 or WO22207839.

In some embodiments, the immunogenic composition comprises a PIV1 F protein mutant or a nucleic acid, preferably a modRNA, encoding a PIV1 F protein mutant described in the disclosure, a hMPV A antigen and a hMPV B antigen. In some embodiments, the hMPV A antigen is selected from mutants of a wild-type hMPV A F protein and a nucleic acids encoding a mutant of a wild-type hMPV A F protein disclosed in any of WO16103238, WO20234300, WO21222639, WO22076669, WO22214678, WO23102373, WO23110618, WO23217988 and WO23102388. In some embodiments, the hMPV A antigen is a mutant of a wild-type hMPV A F protein or a nucleic acid encoding a mutant of a wild-type hMPV A F protein comprising the mutations of mutant 115-BV as disclosed in Battles et al, Nature communication 8:1528 (2017). In some embodiments, the hMPV B antigen is selected from mutants of a wild-type hMPV B F protein and a nucleic acids encoding a mutant of a wild-type hMPV B F protein disclosed in any of WO16103238, WO20234300, WO21222639, WO22076669, WO22214678, WO23102373, WO23110618, WO23217988 and WO23102388. In some embodiments, the hMPV B antigen is a mutant of a wild-type hMPV B F protein or a nucleic acid encoding a mutant of a wild-type hMPV B F protein comprising the mutations of mutant 115-BV as disclosed in Battles et al, Nature communication 8:1528 (2017).

In some embodiments, the immunogenic composition comprises a PIV1 F protein mutant or a nucleic acid, preferably a modRNA, encoding a PIV1 F protein mutant described in the disclosure, a hMPV A F protein mutant or a nucleic acid, preferably a modRNA, encoding a hMPV A F protein mutant described in the disclosure and a hMPV B protein mutant or a nucleic acid, preferably a modRNA, encoding a hMPV B F protein mutant described in the disclosure.

In some embodiments, the immunogenic composition comprises a PIV1 protein mutant or a nucleic acid, preferably a modRNA, encoding a PIV1 F protein mutant described in the disclosure, a hMPV A antigen, a hMPV B antigen and a PIV3 antigen. In some embodiments, the hMPV A antigen is selected from mutants of a wild-type hMPV A F protein and a nucleic acids encoding a mutant of a wild-type hMPV A F protein disclosed in any of WO16103238, WO20234300, WO21222639, WO22076669, WO22214678, WO23102373, WO23110618, WO23217988 and WO23102388. In some embodiments, the hMPV A antigen is a mutant of a wild-type hMPV A F protein or a nucleic acid encoding a mutant of a wild-type hMPV A F protein comprising the mutations of mutant 115-BV as disclosed in Battles et al, Nature communication 8:1528 (2017). In some embodiments, the hMPV B antigen is selected from mutants of a wild-type hMPV B F protein and a nucleic acids encoding a mutant of a wild-type hMPV B F protein disclosed in any of WO16103238, WO20234300, WO21222639, WO22076669, WO22214678, WO23102373, WO23110618, WO23217988 and WO23102388. In some embodiments, the hMPV B antigen is a mutant of a wild-type hMPV B F protein or a nucleic acid encoding a mutant of a wild-type hMPV B F protein comprising the mutations of mutant 115-BV as disclosed in Battles et al, Nature communication 8:1528 (2017).

In some embodiments, the immunogenic composition comprises a PIV1 F protein mutant or a nucleic acid, preferably a modRNA, encoding a PIV1 F protein mutant described in the disclosure, a hMPV A F protein mutant or a nucleic acid, preferably a modRNA, encoding a hMPV A F protein mutant described in the disclosure, a hMPV B protein mutant or a nucleic acid, preferably a modRNA, encoding a hMPV B F protein mutant described in the disclosure and a PIV3 F protein mutant or a nucleic acid, preferably a modRNA, encoding a PIV3 F protein mutant described in the disclosure or disclosed in WO2018081289 or WO22207839.

In some embodiments, the immunogenic composition comprises a PIV3 F protein mutant or a nucleic acid, preferably a modRNA, encoding a PIV3 F protein mutant described in the disclosure.

In some embodiments, the immunogenic composition comprises a PIV3 F protein mutant or a nucleic acid, preferably a modRNA, encoding a PIV3 F protein mutant described in the disclosure and a hMPV A antigen. In some embodiments, the hMPV A antigen is selected from mutants of a wild-type hMPV A F protein and a nucleic acids encoding a mutant of a wild-type hMPV A F protein disclosed in any of WO16103238, WO20234300, WO21222639, WO22076669, WO22214678, WO23102373, WO23110618, WO23217988 and WO23102388. In some embodiments, the hMPV A antigen is a mutant of a wild-type hMPV A F protein or a nucleic acid encoding a mutant of a wild-type hMPV A F protein comprising the mutations of mutant 115-BV as disclosed in Battles et al, Nature communication 8:1528 (2017).

In some embodiments, the immunogenic composition comprises a PIV3 F protein mutant or a nucleic acid, preferably a modRNA, encoding a PIV3 F protein mutant described in the disclosure and a hMPV A F protein mutant or a nucleic acid, preferably a modRNA, encoding a hMPV A F protein mutant described in the disclosure.

In some embodiments, the immunogenic composition comprises a PIV3 F protein mutant or a nucleic acid, preferably a modRNA, encoding a PIV3 F protein mutant described in the disclosure and a hMPV B antigen. In some embodiments, the hMPV B antigen is selected from mutants of a wild-type hMPV B F protein and a nucleic acids encoding a mutant of a wild-type hMPV B F protein disclosed in any of WO16103238, WO20234300, WO21222639, WO22076669, WO22214678, WO23102373, WO23110618, WO23217988 and WO23102388. In some embodiments, the hMPV B antigen is a mutant of a wild-type hMPV B F protein or a nucleic acid encoding a mutant of a wild-type hMPV B F protein comprising the mutations of mutant 115-BV as disclosed in Battles et al, Nature communication 8:1528 (2017).

In some embodiments, the immunogenic composition comprises a PIV3 F protein mutant or a nucleic acid, preferably a modRNA, encoding a PIV3 F protein mutant described in the disclosure and a hMPV B F protein mutant or a nucleic acid, preferably a modRNA, encoding a hMPV B F protein mutant described in the disclosure.

In some embodiments, the immunogenic composition comprises a PIV3 F protein mutant or a nucleic acid, preferably a modRNA, encoding a PIV3 F protein mutant described in the disclosure and a PIV1 antigen.

In some embodiments, the immunogenic composition comprises a PIV3 F protein mutant or a nucleic acid, preferably a modRNA, encoding a PIV3 F protein mutant described in the disclosure and a PIV1 F protein mutant or a nucleic acid, preferably a modRNA, encoding a PIV3 F protein mutant described in the disclosure.

In some embodiments, the immunogenic composition comprises a PIV3 F protein mutant or a nucleic acid, preferably a modRNA, encoding a PIV3 F protein mutant described in the disclosure, a hMPV B antigen and a PIV1 antigen. In some embodiments, the hMPV B antigen is selected from mutants of a wild-type hMPV B F protein and a nucleic acids encoding a mutant of a wild-type hMPV B F protein disclosed in any of WO16103238, WO20234300, WO21222639, WO22076669, WO22214678, WO23102373, WO23110618, WO23217988 and WO23102388. In some embodiments, the hMPV B antigen is a mutant of a wild-type hMPV B F protein or a nucleic acid encoding a mutant of a wild-type hMPV B F protein comprising the mutations of mutant 115-BV as disclosed in Battles et al, Nature communication 8:1528 (2017)

In some embodiments, the immunogenic composition comprises a PIV3 F protein mutant or a nucleic acid, preferably a modRNA, encoding a PIV3 F protein mutant described in the disclosure, a hMPV B protein mutant or a nucleic acid, preferably a modRNA, encoding a hMPV B F protein mutant described in the disclosure and a PIV1 protein mutant or a nucleic acid, preferably a modRNA, encoding a PIV1 F protein mutant described in the disclosure.

In some embodiments, the immunogenic composition comprises a PIV3 F protein mutant or a nucleic acid, preferably a modRNA, encoding a PIV3 F protein mutant described in the disclosure, a hMPV A antigen antigen and a PIV1 antigen. In some embodiments, the hMPV A antigen is selected from mutants of a wild-type hMPV A F protein and a nucleic acids encoding a mutant of a wild-type hMPV A F protein disclosed in any of WO16103238, WO20234300, WO21222639, WO22076669, WO22214678, WO23102373, WO23110618, WO23217988 and WO23102388. In some embodiments, the hMPV A antigen is a mutant of a wild-type hMPV A F protein or a nucleic acid encoding a mutant of a wild-type hMPV A F protein comprising the mutations of mutant 115-BV as disclosed in Battles et al, Nature communication 8:1528 (2017).

In some embodiments, the immunogenic composition comprises a PIV3 F protein mutant or a nucleic acid, preferably a modRNA, encoding a PIV3 F protein mutant described in the disclosure, a hMPV A F protein mutant or a nucleic acid, preferably a modRNA, encoding a hMPV A F protein mutant described in the disclosure and a PIV1 protein mutant or a nucleic acid, preferably a modRNA, encoding a PIV1 F protein mutant described in the disclosure.

In some embodiments, the immunogenic composition comprises a PIV3 F protein mutant or a nucleic acid, preferably a modRNA, encoding a PIV3 F protein mutant described in the disclosure, a hMPV A antigen and a hMPV B antigen. In some embodiments, the hMPV A antigen is selected from mutants of a wild-type hMPV A F protein and a nucleic acids encoding a mutant of a wild-type hMPV A F protein disclosed in any of WO16103238, WO20234300, WO21222639, WO22076669, WO22214678, WO23102373, WO23110618, WO23217988 and WO23102388. In some embodiments, the hMPV A antigen is a mutant of a wild-type hMPV A F protein or a nucleic acid encoding a mutant of a wild-type hMPV A F protein comprising the mutations of mutant 115-BV as disclosed in Battles et al, Nature communication 8:1528 (2017). In some embodiments, the hMPV B antigen is selected from mutants of a wild-type hMPV B F protein and a nucleic acids encoding a mutant of a wild-type hMPV B F protein disclosed in any of WO16103238, WO20234300, WO21222639, WO22076669, WO22214678, WO23102373, WO23110618, WO23217988 and WO23102388. In some embodiments, the hMPV B antigen is a mutant of a wild-type hMPV B F protein or a nucleic acid encoding a mutant of a wild-type hMPV B F protein comprising the mutations of mutant 115-BV as disclosed in Battles et al, Nature communication 8:1528 (2017).

In some embodiments, the immunogenic composition comprises a PIV3 F protein mutant or a nucleic acid, preferably a modRNA, encoding a PIV3 F protein mutant described in the disclosure, a hMPV A F protein mutant or a nucleic acid, preferably a modRNA, encoding a hMPV A F protein mutant described in the disclosure and a hMPV B protein mutant or a nucleic acid, preferably a modRNA, encoding a hMPV B F protein mutant described in the disclosure.

In some embodiments, the immunogenic composition comprises a PIV3 F protein mutant or a nucleic acid, preferably a modRNA, encoding a PIV3 F protein mutant described in the disclosure, a hMPV A antigen, a hMPV B antigen and a PIV1 antigen. In some embodiments, the hMPV A antigen is selected from mutants of a wild-type hMPV A F protein and a nucleic acids encoding a mutant of a wild-type hMPV A F protein disclosed in any of WO16103238, WO20234300, WO21222639, WO22076669, WO22214678, WO23102373, WO23110618, WO23217988 and WO23102388. In some embodiments, the hMPV A antigen is a mutant of a wild-type hMPV A F protein or a nucleic acid encoding a mutant of a wild-type hMPV A F protein comprising the mutations of mutant 115-BV as disclosed in Battles et al, Nature communication 8:1528 (2017). In some embodiments, the hMPV B antigen is selected from mutants of a wild-type hMPV B F protein and a nucleic acids encoding a mutant of a wild-type hMPV B F protein disclosed in any of WO16103238, WO20234300, WO21222639, WO22076669, WO22214678, WO23102373, WO23110618, WO23217988 and WO23102388. In some embodiments, the hMPV B antigen is a mutant of a wild-type hMPV B F protein or a nucleic acid encoding a mutant of a wild-type hMPV B F protein comprising the mutations of mutant 115-BV as disclosed in Battles et al, Nature communication 8:1528 (2017).

In some embodiments, the immunogenic composition comprises a PIV3 F protein mutant or a nucleic acid, preferably a modRNA, encoding a PIV3 F protein mutant described in the disclosure, a hMPV A F protein mutant or a nucleic acid, preferably a modRNA, encoding a hMPV A F protein mutant described in the disclosure, a hMPV B protein mutant or a nucleic acid, preferably a modRNA, encoding a hMPV B F protein mutant described in the disclosure and a PIV1 protein mutant or a nucleic acid, preferably a modRNA, encoding a PIV1 F protein mutant described in the disclosure.

In some embodiments, the immunogenic composition further comprises an RSV antigen selected from the group consisting of a mutant of a wild-type RSV F protein of subtype A and a nucleic acid, preferably modRNA encoding a mutant of a wild-type RSV F protein of subtype A. Preferably, the mutant is in the form of a trimer. Preferably, the mutant is in the prefusion conformation. Preferably, the mutant is in the prefusion conformation and is in the form of a trimer.

Preferably, the RSV antigen is disclosed in one of WO2009/079796, WO2010/149745, WO2011/008974, WO2014/160463, WO2014/174018, WO2014/202570, WO2015/013551, WO2015/177312, WO2017/005848, WO2017/174564, WO2017/005844, WO2017/109629, WO2022/002894 and WO2018/109220. In some embodiment, the RSV antigen is a mutant of a wild-type RSV F protein of subtype A or a nucleic acid, preferably modRNA, encoding a mutant of a wild-type RSV F protein of subtype A comprising the mutations T103C, 1148C, S1901, and D486S.

In some embodiments, the composition further comprises an RSV antigen selected from the group consisting of a mutant of a wild-type RSV F protein of subtype B and a nucleic acid, preferably modRNA encoding a mutant of a wild-type RSV F protein of subtype B. Preferably, the mutant is in the form of a trimer. Preferably, the mutant is in the prefusion conformation. Preferably, the mutant is in the prefusion conformation and is in the form of a trimer. Preferably, the RSV antigen is disclosed in one of WO2009/079796, WO2010/149745, WO2011/008974, WO2014/160463, WO2014/174018, WO2014/202570, WO2015/013551, WO2015/177312, WO2017/005848, WO2017/174564, WO2017/005844, WO2017/109629, WO2022/002894 and WO2018/109220. In some embodiment, the RSV antigen is a mutant of a wild-type RSV F protein of subtype B or a nucleic acid, preferably modRNA, encoding a mutant of a wild-type RSV F protein of subtype B comprising the mutations T103C, 1148C, S1901, and D486S.

In some embodiments, the composition further comprises an RSV A antigen selected from the group consisting of a mutant of a wild-type RSV F protein of subtype A and a nucleic acid, preferably modRNA encoding a mutant of a wild-type RSV F protein of subtype A and an RSV B antigen selected from the group consisting of a mutant of a wild-type RSV F protein of subtype B and a nucleic acid, preferably modRNA encoding a mutant of a wild-type RSV F protein of subtype B. Preferably, the mutants are in the form of a trimer. Preferably, the mutants are in the prefusion conformation. Preferably, the mutants are in the prefusion conformation and is in the form of a trimer. Preferably, the RSV A and B antigens are disclosed in one of WO2009/079796, WO2010/149745, WO2011/008974, WO2014/160463, WO2014/174018, WO2014/202570, WO2015/013551, WO2015/177312, WO2017/005848, WO2017/174564, WO2017/005844, WO2017/109629, WO2022/002894 and WO2018/109220. In some embodiment, the RSV A antigen is a mutant of a wild-type RSV F protein of subtype A or a nucleic acid, preferably modRNA, encoding a mutant of a wild-type RSV F protein of subtype A comprising the mutations T103C, 1148C, S1901, and D486S and the RSV B antigen is a mutant of a wild-type RSV F protein of subtype B or a nucleic acid, preferably modRNA, encoding a mutant of a wild-type RSV F protein of subtype B comprising the mutations T103C, 1148C, S1901, and D486S.

In some embodiments, the immunogenic composition is capable of eliciting an immune response against the F protein of hMPV A, hMPV B, PIV1 or PIV3 in the prefusion conformation in a subject.

In some embodiments, the immunogenic composition further comprises a pharmaceutically acceptable carrier.

In some embodiments, the immunogenic composition is a vaccine.

In addition to the immunogenic component, the vaccine may further comprise an immunomodulatory agent, such as an adjuvant. Examples of suitable adjuvants include aluminum salts such as aluminum hydroxide and/or aluminum phosphate; oil-emulsion compositions (or oil-in-water compositions), including squalene-water emulsions, such as MF59 (see e.g., WO 90/14837); saponin formulations, such as, for example, QS21 and Immunostimulating Complexes (ISCOMS) (see e.g., U.S. Pat. No. 5,057,540; WO 90/03184, WO 96/11711, WO 2004/004762, WO 2005/002620); bacterial or microbial derivatives, examples of which are monophosphoryl lipid A (MPL), 3-O-deacylated MPL (3dMPL), CpG-motif containing oligonucleotides, ADP-ribosylating bacterial toxins or mutants thereof, such as E. coli heat labile enterotoxin LT, cholera toxin CT, and the like. It is also possible to use vector-encoded adjuvant, e.g., by using heterologous nucleic acid that encodes a fusion of the oligomerization domain of C4-binding protein (C4 bp) to the antigen of interest (e.g., Solabomi et al., 2008, Infect Immun 76: 3817-23). In certain embodiments the compositions hereof comprise aluminum as an adjuvant, e.g., in the form of aluminum hydroxide, aluminum phosphate, aluminum potassium phosphate, or combinations thereof, in concentrations of 0.05-5 mg, e.g., from 0.075-1.0 mg, of aluminum content per dose.

F. Uses of the hMPV A, hMPV B, PIV1 and/or PIV3 F Protein Mutants, Nucleic Acid Molecules, and Compositions

The present disclosure also relates to use of a hMPV A, hMPV B, PIV1 or PIV3 F protein mutant disclosed herein, nucleic acids encoding a hMPV A, hMPV B, PIV1 or PIV3 F protein mutant disclosed herein, or vectors for expressing a hMPV A, hMPV B, PIV1 or PIV3 F protein mutant disclosed herein, or compositions comprising a hMPV A, hMPV B, PIV1 or PIV3 F protein mutant or nucleic acids disclosed herein.

In several embodiments, the present disclosure provides a method of eliciting an immune response to hMPV A, hMPV B, PIV1 and/or PIV3 in a subject, comprising administering to the subject an effective amount of a hMPV A, hMPV B, PIV1 and/or PIV3 F protein mutant disclosed herein, a nucleic acid molecule encoding a hMPV A, hMPV B, PIV1 and/or PIV3 F protein mutant disclosed herein, or a composition comprising a hMPV A, hMPV B, PIV1 and/or PIV3 F protein mutant or nucleic acid molecule disclosed herein.

In some particular embodiments, the present disclosure provides a method of preventing hMPV A, hMPV B, PIV1 and/or PIV3 infection in a subject, comprising administering to the subject an effective amount of a pharmaceutical composition, such as a vaccine, comprising a hMPV A, hMPV B, PIV1 and/or PIV3 F protein mutant disclosed herein, a nucleic acid encoding a hMPV A, hMPV B, PIV1 and/or PIV3 F protein mutant disclosed herein, or a vector expressing a hMPV A, hMPV B, PIV1 and/or PIV3 F protein mutant disclosed herein. In some embodiments, the subject is a human. In some particular embodiments, the human is a child, such as an infant. In some other particular embodiments, the human is a woman, particularly a pregnant woman.

In several embodiments, the present disclosure provides an hMPV A, hMPV B, PIV1 and/or PIV3 F protein mutant disclosed herein, a nucleic acid molecule encoding a hMPV A, hMPV B, PIV1 and/or PIV3 F protein mutant disclosed herein, or a composition comprising a hMPV A, hMPV B, PIV1 and/or PIV3 F protein mutant or nucleic acid molecule disclosed herein for use as a vaccine.

In several embodiments, the present disclosure provides the use of hMPV A, hMPV B, PIV1 and/or PIV3 F protein mutant disclosed herein, a nucleic acid molecule encoding a hMPV A, hMPV B, PIV1 and/or PIV3 F protein mutant disclosed herein, or a composition comprising a hMPV A, hMPV B, PIV1 and/or PIV3 F protein mutant or nucleic acid molecule disclosed herein for the manufacture of a medicament, preferably a vaccine.

In several embodiments, the present disclosure provides an hMPV A, hMPV B, PIV1 and/or PIV3 F protein mutant disclosed herein, a nucleic acid molecule encoding a hMPV A, hMPV B, PIV1 and/or PIV3 F protein mutant disclosed herein, or a composition comprising a hMPV A, hMPV B, PIV1 and/or PIV3 F protein mutant or nucleic acid molecule disclosed herein for use in a method of eliciting an immune response to hMPV A, hMPV B, PIV1 and/or PIV3 in a subject, said method comprising administering to the subject an effective amount of said protein mutant, nucleic acid molecule or composition.

In several embodiments, the present disclosure provides an hMPV A, hMPV B, PIV1 and/or PIV3 F protein mutant disclosed herein, a nucleic acid molecule encoding a hMPV A, hMPV B, PIV1 and/or PIV3 F protein mutant disclosed herein, or a composition comprising a hMPV A, hMPV B, PIV1 and/or PIV3 F protein mutant or nucleic acid molecule disclosed herein for use in preventing hMPV A, hMPV B, PIV1 and/or PIV3 infection in a subject, said method comprising administering to the subject an effective amount of said protein mutant, nucleic acid molecule or composition.

In some embodiments, the subject is a human. In some particular embodiments, the human is a child, such as an infant. In some other particular embodiments, the human is a woman, particularly a pregnant woman.

The composition may be administered to the subject with or without administration of an adjuvant. The effective amount administered to the subject is an amount that is sufficient to elicit an immune response against an hMPV A, hMPV B, PIV1 and/or PIV3 antigen, such as hMPV A, hMPV B, PIV1 and/or PIV3 F protein, in the subject. Subjects that can be selected for treatment include those that are at risk for developing an hMPV A, hMPV B, PIV1 and/or PIV3 infection because of exposure or the possibility of exposure to hMPV A, hMPV B, PIV1 and/or PIV3. Because nearly all humans are infected with hMPV A, hMPV B, PIV1 and/or PIV3 by the age of 5, the entire birth cohort is included as a relevant population for immunization. This could be done, for example, by beginning an immunization regimen anytime from birth to 6 months of age, from 6 months of age to 5 years of age, in pregnant women (or women of child-bearing age) to protect their infants by passive transfer of antibody, family members of newborn infants or those still in utero, and subjects greater than 50 years of age. Subjects at greatest risk of hMPV A, hMPV B, PIV1 and/or PIV3 infection with severe symptoms (e.g. requiring hospitalization) include children with prematurity, bronchopulmonary dysplasia, and congenital heart disease.

Administration of the compositions provided by the present disclosure, such as pharmaceutical compositions, can be carried out using standard routes of administration. Non-limiting embodiments include parenteral administration, such as intradermal, intramuscular, subcutaneous, transcutaneous, mucosal, or oral administration.

The total dose of the composition provided to a subject during one administration can be varied as is known to the skilled practitioner.

It is also possible to provide one or more booster administrations of one or more of the vaccine compositions. If a boosting vaccination is performed, typically, such a boosting vaccination will be administered to the same subject at a moment between one week and 10 years, preferably between two weeks and six months, after administering the composition to the subject for the first time (which is in such cases referred to as “priming vaccination”). In alternative boosting regimens, it is also possible to administer different vectors, e.g., one or more adenovirus, or other vectors such as modified vaccinia virus of Ankara (MVA), or DNA, or protein, to the subject after the priming vaccination. It is, for instance, possible to administer to the subject a recombinant viral vector hereof as a prime, and boosting with a composition comprising hMPV A, hMPV B, PIV1 and/or PIV3 F protein.

In certain embodiments, the administration comprises a priming administration and at least one booster administration. In certain other embodiments, the administration is provided annually. In still other embodiments, the administration is provided annually together with an influenza vaccine.

The vaccines provided by the present disclosure may be used together with one or more other vaccines. For example, in adults they may be used together with an influenza vaccine, Prevnar, tetanus vaccine, diphtheria vaccine, RSV vaccine such as Abryvso™ or Arexvy™, COVID19 vaccine and pertussis vaccine. For pediatric use, vaccines provided by the present disclosure may be used with any other vaccine indicated for pediatric patients.

### G. RNA Molecule

In some aspects of the present disclosure, an RNA is or comprises messenger RNA (mRNA) that relates to an RNA transcript which encodes a polypeptide. In some aspects, an RNA disclosed herein comprises: a 5′ cap disclosed herein; a 5′ untranslated region comprising a cap proximal sequence (5′ UTR), a sequence encoding a payload (e.g., a hMPV F protein mutant and/or an antigen derived from PIV1 and/or an antigen derived from PIV3); a 3′ untranslated region (3′ UTR); and a polyadenylate (Poly A) sequence. In some aspects, an RNA disclosed herein comprises the following components in 5′ to 3′ orientation: a 5′ cap comprising a 5′ cap disclosed herein; a 5′ untranslated region comprising a cap proximal sequence (5′ UTR), a sequence encoding a payload (e.g., a hMPV F protein mutant and/or an antigen derived from PIV1 and/or an antigen derived from PIV3)); a 3′ untranslated region (3′ UTR); and a Poly-A sequence.

### 1. Modified Nucleobases

In the present disclosure the RNA molecules may comprise modified nucleobases which may be incorporated into modified nucleosides and nucleotides. In some aspects, the RNA molecule may include one or more modified nucleotides. Naturally occurring nucleotide modifications are known in the art.

In some aspects, the RNA molecule may include a modified nucleotide. Non-limiting examples of modified nucleotides that may be included in the RNA molecule include pseudouridine, N1-methylpseudouridine, 5-methyluridine, 3-methyl-uridine, 5-methoxy-uridine, 5-aza-uridine, 6-aza-uridine, 2-thio-5-aza-uridine, 2-thio-uridine, 4-thio-uridine, 4-thio-pseudouridine, 2-thio-pseudouridine, 5-hydroxy-uridine, 5-aminoallyl-uridine, 5-halo-uridine (e.g., 5-iodo-uridine or 5-bromo-uridine), uridine 5-oxyacetic acid, uridine 5-oxyacetic acid methyl ester, 5-carboxymethyl-uridine, 1-carboxymethyl-pseudouridine, 5-carboxy hydroxymethyl-uridine, 5-carboxy hydroxy methyl-uridine methyl ester, 5-methoxycarbonylmethyl-uridine, 5-methoxycarbonylmethyl-2-thio-uridine, 5-aminomethyl-2-thio-uridine, 5-methylaminomethyl-uridine, 1-ethyl-pseudouridine, 5-methylaminomethyl-2-thio-uridine, 5-methylaminomethyl-2-seleno-uridine, 5-carbamoylmethyl-uridine, 5-carboxymethylaminomethyl-uridine, 5-carboxymethylaminomethyl-2-thio-uridine, 5-propynyl-uridine, 1-propynyl-pseudouridine, 5-taurinomethyl-uridine, 1-taurinomethyl-pseudouridine, 5-taurinomethyl-2-thio-uridine, 1-taurinomethyl-4-thio-pseudouridine, 5-methyl-2-thio-uridine, 1-methyl-4-thio-pseudouridine, 4-thio-1-methyl-pseudouridine, 3-methyl-1-pseudouridine, 2-thio-1-methyl-pseudouridine, 1-methyl-1-deaza-pseudouridine, 2-thio-1-methyl-1-deaza-pseudouridine, dihydrouridine, dihydropseudouridine, 5,6-dihydrouridine, 5-methyl-dihydrouridine, 2-thio-dihydrouridine, 2-thio-dihydropseudouridine, 2-methoxy-uridine, 2-methoxy-4-thio-uridine, 4-methoxy-pseudouridine, 4-methoxy-2-thio-pseudouridine, N1-methyl-pseudouridine, 3-(3-amino-3-carboxypropyl)uridine, 1-methyl-3-(3-amino-3-carboxypropyl)pseudouridine, 5-(isopentenylaminomethyl)uridine, 5-(isopentenylaminomethyl)-2-thio-uridine, α-thio-uridine, 2′-O-methyl-uridine, 5,2′-O-dimethyl-uridine, 2′-O-methyl-pseudouridine, 2-thio-2′-O-methyl-uridine, 5-methoxycarbonylmethyl-2′-O-methyl-uridine, 5-carbamoylmethyl-2′-O-methyl-uridine, 5-carboxymethylaminomethyl-2′-O-methyl-uridine, 3,2′-O-dimethyl-uridine, 5-(isopentenylaminomethyl)-2′-O-methyl-uridine, 1-thio-uridine, deoxythymidine, 2′-F-ara-uridine, 2′-F-uridine, 2′-OH-ara-uridine, 5-(2-carbomethoxyvinyl) uridine, 5-[3-(1-E-propenylamino)uridine, any other modified uridine known in the art, or combinations thereof. In some aspects of the present disclosure, modified nucleotides include any one of N1-methylpseudouridine or pseudouridine.

In some aspects, the RNA molecule comprises nucleotides that are N1-methylpseudouridine modified. In some aspects, the RNA molecule comprises nucleotides that are a pseudouridine modified.

In some aspects, an RNA comprises a modified nucleoside in place of at least one uridine. In some aspects, an RNA comprises a modified nucleoside in place of each uridine. In some aspects, the RNA molecule comprises a sequence having at least one uridine replaced by N1-methylpseudouridine. In some aspects, the RNA molecule comprises a sequence having all uridines replaced by N1-methylpseudouridine. N1-methylpseudouridine is designated in sequences as “4”. The term “uracil,” as used herein, describes one of the nucleobases that may occur in the nucleic acid of RNA. The term “uridine,” as used herein, describes one of the nucleosides that may occur in RNA. “Pseudouridine” is one example of a modified nucleoside that is an isomer of uridine, where the uracil is attached to the pentose ring via a carbon-carbon bond instead of a nitrogen-carbon glycosidic bond.

In some aspects, the RNA molecule comprises a nucleic acid sequence having at least one uridine replaced by pseudouridine. In some aspects, the RNA molecule comprises a nucleic acid sequence having at least, at most, exactly, or between any two of 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% of uridines replaced by pseudouridine. In some aspects, the RNA molecule comprises a nucleic acid sequence having all uridines replaced by pseudouridine.

Modifications that may be present in the RNA molecules further include, for example, m5C (5-methylcytidine), m5U (5-methyluridine), m6A (N6-methyladenosine), s2U (2-thiouridine), Um (2′-O-methyluridine), m1A (1-methyladenosine); m2A (2-methyladenosine); Am (2-1-O-methyladenosine); ms2m6A (2-methylthio-N6-methyladenosine); i6A (N6-isopentenyladenosine); ms2i6A (2-methylthio-N6 isopentenyladenosine); io6A (N6-(cis-hydroxyisopentenyl)adenosine); ms2io6A (2-methylthio-N6-(cis-hydroxyisopentenyl) adenosine); g6A (N6-glycinylcarbamoyladenosine); t6A (N6-threonyl carbamoyladenosine); ms2t6A (2-methylthio-N6-threonyl carbamoyladenosine); m6t6A (N6-methyl-N6-threonylcarbamoyladenosine); hn6A (N6-hydroxynorvalylcarbamoyl adenosine); ms2hn6A (2-methylthio-N6-hydroxynorvalyl carbamoyladenosine); Ar(p) (2′-O-ribosyladenosine (phosphate)); I (inosine); mil (1-methylinosine); m′Im (1,2′-O-dimethylinosine); m3C (3-methylcytidine); Cm (2T-O-methylcytidine); s2C (2-thiocytidine); ac4C (N4-acetylcytidine); f5C (5-formylcytosine); m5Cm (5,2-O-dimethylcytidine); ac4Cm (N4acetyl2TOmethylcytidine); k2C (lysidine); m1G (1-methylguanosine); m2G (N2-methylguanosine); m7G (7-methylguanosine); Gm (2′-O-methylguanosine); m22G (N2,N2-dimethylguanosine); m2Gm (N2,2′-O-dimethylguanosine); m22Gm (N2,N2,2′-O-trimethylguanosine); Gr(p) (2′-O-ribosylguanosine (phosphate)); yW (wybutosine); o2yW (peroxywybutosine); OHyW (hydroxywybutosine); OHyW*(undermodified hydroxywybutosine); imG (wyosine); mimG (methylguanosine); Q (queuosine); oQ (epoxyqueuosine); galQ (galtactosyl-queuosine); manQ (mannosyl-queuosine); preQo (7-cyano-7-deazaguanosine); preQi (7-aminomethyl-7-deazaguanosine); G* (archaeosine); D (dihydrouridine); m5Um (5,2′-O-dimethyluridine); s4U (4-thiouridine); m5s2U (5-methyl-2-thiouridine); s2Um (2-thio-2′-O-methyluridine); acp3U (3-(3-amino-3-carboxypropyl)uridine); ho5U (5-hydroxyuridine); mo5U (5-methoxyuridine); cmo5U (uridine 5-oxyacetic acid); mcmo5U (uridine 5-oxyacetic acid methyl ester); chm5U (5-(carboxyhydroxymethyl)uridine)); mchm5U (5-(carboxyhydroxymethyl)uridine methyl ester); mcm5U (5-methoxycarbonyl methyluridine); mcm5Um (S-methoxycarbonylmethyl-2-O-methyluridine); mcm5s2U (5-methoxycarbonylmethyl-2-thiouridine); nm5s2U (5-aminomethyl-2-thiouridine); mnm5U (5-methylaminomethyluridine); mnm5s2U (5-methylaminomethyl-2-thiouridine); mnm5se2U (5-methylaminomethyl-2-selenouridine); ncm5U (5-carbamoylmethyl uridine); ncm5Um (5-carbamoylmethyl-2′-O-methyluridine); cmnm5U (5-carboxymethylaminomethyluridine); cnmm5Um (5-carboxymethy 1 aminomethyl-2-L-O-methyluridine); cmnm5s2U (5-carboxymethylaminomethyl-2-thiouridine); m62A (N6,N6-dimethyladenosine); Tm (2′-O-methylinosine); m4C (N4-methylcytidine); m4Cm (N4,2-O-dimethylcytidine); hm5C (5-hydroxymethylcytidine); m3U (3-methyluridine); cm5U (5-carboxymethyluridine); m6Am (N6,T-O-dimethyladenosine); rn62Am (N6,N6,O-2-trimethyladenosine); m2′7G (N2,7-dimethylguanosine); m2′2′7G (N2,N2,7-trimethylguanosine); m3Um (3,2T-O-dimethyluridine); m5D (5-methyldihydrouridine); f5Cm (5-formyl-2′-O-methylcytidine); m1Gm (1,2′-O-dimethylguanosine); m′Am (1,2-O-dimethyl adenosine) irinomethyluridine); tm5s2U (S-taurinomethyl-2-thiouridine)); imG-14 (4-demethyl guanosine); imG2 (isoguanosine); ac6A (N6-acetyladenosine), hypoxanthine, inosine, 8-oxo-adenine, 7-substituted derivatives thereof, dihydrouracil, pseudouracil, 2-thiouracil, 4-thiouracil, 5-aminouracil, 5-(C1-C6)-alkyluracil, 5-methyluracil, 5-(C2-Ce)-alkenyluracil, 5-(C2-Ce)-alkynyluracil, 5-(hydroxymethyl)uracil, 5-chlorouracil, 5-fluorouracil, 5-bromouracil, 5-hydroxycytosine, 5-(C1-C6)-alkylcytosine, 5-methylcytosine, 5-(C2-C6)-alkenylcytosine, 5-(C2-C6)-alkynylcytosine, 5-chlorocytosine, 5-fluorocytosine, 5-bromocytosine, N2-dimethylguanine, 7-deazaguanine, 8-azaguanine, 7-deaza-7-substituted guanine, 7-deaza-7-(C2-C6)alkynylguanine, 7-deaza-8-substituted guanine, 8-hydroxyguanine, 6-thioguanine, 8-oxoguanine, 2-aminopurine, 2-amino-6-chloropurine, 2,4-diaminopurine, 2,6-diaminopurine, 8-azapurine, substituted 7-deazapurine, 7-deaza-7-substituted purine, 7-deaza-8-substituted purine, hydrogen (abasic residue), m5C, m5U, m6A, s2U, W, or 2′-O-methyl-U. In some aspects, the RNA molecule may include phosphoramidate, phosphorothioate, and/or methylphosphonate linkages.

The sequence of the RNA molecule may be modified if desired, for example to increase the efficacy of expression or replication of the RNA, or to provide additional stability or resistance to degradation. For example, the RNA sequence may be modified with respect to its codon usage, for example, to increase translation efficacy and half-life of the RNA.

In some aspects, the RNA molecule of the present disclosure comprises an open reading frame having at least one codon modified sequence. A codon modified sequence relates to coding sequences that differ in at least one codon (triplets of nucleotides coding for one amino acid) compared to the corresponding wild type coding sequence. A codon modified sequence may show improved resistance to degradation, improved stability, and/or improved translatability.

The sequence of the RNA molecule may be codon optimized or deoptimized for expression in a desired host, such as a human cell.

In some aspects, the RNA molecule may include one or more modified nucleotides in addition to any 5′ cap structure. Naturally occurring nucleotide modifications are known in the art.

In some aspects, the RNA molecule does not include modified nucleotides, e.g., does not include modified nucleobases, and all of the nucleotides in the RNA molecule are conventional standard ribonucleotides A, U, G and C, with the exception of an optional 5′ cap that may include, for example, 7-methylguanosine, which is further described below. In some aspects, the RNA may include a 5′ cap comprising a 7′-methylguanosine, and the first 1, 2 or 3 5′ ribonucleotides may be methylated at the 2′ position of the ribose.

In some aspects, the RNA molecule described herein is a non-coding RNA molecule. A non-coding RNA (ncRNA) molecule includes a functional RNA molecule that is not translated into a peptide or polypeptide. Non-coding RNA molecules may include highly abundant and functionally important RNA molecules. In some aspects, the non-coding RNA is a functional mRNA molecule that is not translated into a peptide or polypeptide. The non-coding RNA may include modified nucleotides as described herein. Preferably, the RNA molecule is an mRNA The RNA molecules of the present disclosure may be prepared by any method know in the art, including chemical synthesis and in vitro methods, such as RNA in vitro transcription.

In some of the aspects, the RNA of the present disclosure is prepared using in vitro transcription. In some aspects, the RNA molecule of the present disclosure is purified, e.g., such as by filtration that may occur via, e.g., ultrafiltration, diafiltration, or, e.g., tangential flow ultrafiltration/diafiltration. In some aspects, the RNA molecule of the present disclosure is lyophilized to be temperature stable.

### 2. 5′ Cap

In some aspects, the RNA molecule described herein includes a 5′ cap which generally “caps” the 5′ end of the RNA and stabilizes the RNA molecule. In some aspects, the 5′ cap moiety is a natural 5′ cap. A “natural 5′ cap” is defined as a cap that includes 7-methylguanosine connected to the 5′ end of an mRNA molecule through a 5′ to 5′ triphosphate linkage. In some aspects, a guanosine nucleoside included in a 5′ cap may be modified, for example, by methylation at one or more positions (e.g., at the 7-position) on a base (guanine), and/or by methylation at one or more positions of a ribose. In some aspects, a guanosine nucleoside included in a 5′ cap comprises a 3′O methylation at a ribose (3′OMeG). In some aspects, a guanosine nucleoside included in a 5′ cap comprises methylation at the 7-position of guanine (m7G). In some aspects, a guanosine nucleoside included in a 5′ cap comprises methylation at the 7-position of guanine and a 3′O methylation at a ribose (m7(3′OMeG)). The 5′ cap may be incorporated during RNA synthesis (e.g., co-transcriptional capping) or may be enzymatically engineered after RNA transcription (e.g., post-transcriptional capping). In some aspects, co-transcriptional capping with a cap disclosed herein improves the capping efficiency of an RNA compared to co-transcriptional capping with an appropriate reference comparator. In some aspects, improving capping efficiency may increase a translation efficiency and/or translation rate of an RNA, and/or increase expression of an encoded polypeptide. In some aspects, capping is performed after purification, e.g., tangential flow filtration, of the RNA molecule.

In some aspects, an RNA described herein comprises a 5′ cap or a 5′ cap analog, e.g., a Cap 0, a Cap 1 or a Cap 2. In some aspects, a provided RNA does not have uncapped 5′-triphosphates. In some aspects, the 5′ end of the RNA is capped with a modified ribonucleotide. In some aspects, the 5′ cap moiety is a 5′ cap analog. In some aspects, an RNA may be capped with a 5′ cap analog. Cap structures include, but are not limited to, 7mG(5′)ppp(5′)N,pN2p (Cap 0) and 7mG(5′)ppp(5′)N1mpNp (Cap 1). In some aspects, an RNA described herein comprises a Cap 0. Cap 0 is a N7-methyl guanosine connected to the 5′ nucleotide through a 5′ to 5′ triphosphate linkage, typically referred to as m7G cap or m7Gppp. In the cell, the Cap 0 structure is essential for efficient translation of the mRNA that carries the cap. An additional methylation on the 2′O position of the initiating nucleotide generates Cap 1, or referred to as m7GpppNm, wherein Nm denotes any nucleotide with a 2′O methylation. In some aspects, an RNA described herein comprises a Cap 1, e.g., as described herein. In some aspects, an RNA described herein comprises a Cap 2.

In some aspects, a Cap 0 structure comprises a guanosine nucleoside methylated at the 7-position of guanine (m7G). In some aspects, a Cap 0 structure is connected to an RNA via a 5′ to 5′-triphosphate linkage and is also referred to herein as m7Gppp or m7G(5′)ppp(5′). A 5′ cap may be methylated with the structure m7G (5′) ppp (5′) N (cap-0 structure) or a derivative thereof, wherein N is the terminal 5′ nucleotide of the nucleic acid carrying the 5′ cap, typically the 5′-end of an mRNA. An exemplary enzymatic reaction for capping may include use of Vaccinia Virus Capping Enzyme (VCE) that includes mRNA triphosphatase, guanylyl-transferase and guanine-7-methytransferase, which catalyzes the construction of N7-monomethylated Cap 0 structures. Cap 0 structure plays an important role in maintaining the stability and translational efficacy of the RNA molecule.

The 5′ cap of the RNA molecule may be further modified by a 2′-O-Methyltransferase which results in the generation of a Cap 1 structure (m7Gppp [m2′-O] N), which may further increase translation efficacy. In some aspects, a Cap 1 structure comprises a guanosine nucleoside methylated at the 7-position of guanine (m7G) and a 2′O methylated first nucleotide in an RNA (2′OmeN₁). In some aspects, a Cap 1 structure is connected to an RNA via a 5′- to 5′-triphosphate linkage and is also referred to herein as m7Gppp(2′OMeN₁) or m7G(5′)ppp(5′)(2′OMeN₁). In some aspects, N₁ is chosen from A, C, G, or U. In some aspects, N₁ is A. In some aspects, N₁ is C. In some aspects, N₁ is G. In some aspects, N₁ is U. In some aspects, a m7G(5′)ppp(5′)(2′OmeN₁) Cap 1 structure comprises a second nucleotide, N₂, which is a cap proximal nucleotide at position 2 and is chosen from A, G, C, or U (m7G(5′)ppp(5′)(2′OmeN₁)N₂). In some aspects, N₂ is A. In some aspects, N₂ is C. In some aspects, N₂ is G. In some aspects, N₂ is U.

In some aspects, a Cap 1 structure comprises a guanosine nucleoside methylated at the 7-position of guanine (m7G) and one or more additional modifications, e.g., methylation on a ribose, and a 2′O methylated first nucleotide in an RNA. In some aspects, a Cap 1 structure comprises a guanosine nucleoside methylated at the 7-position of guanine, a 3′O methylation at a ribose (m7(3′OMeG)), and a 2′O methylated first nucleotide in an RNA (2′OMeN₁). In some aspects, a Cap 1 structure is connected to an RNA via a 5′- to 5′-triphosphate linkage and is also referred to herein as m7(3′OMeG)ppp(2′OMeN₁) or m7(3′OMeG)(5′)ppp(5′)(2′OMeN₁). In some aspects, N₁ is chosen from A, C, G, or U. In some aspects, N₁ is A. In some aspects, N₁ is C. In some aspects, N₁ is G. In some aspects, N₁ is U. In some aspects, a m7(3′OMeG)(5′)ppp(5′)(2′OMeN₁) Cap 1 structure comprises a second nucleotide, N₂, which is a cap proximal nucleotide at position 2 and is chosen from A, G, C, or U (m7(3′OMeG)(5′)ppp(5′)(2′OmeN₁)N₂). In some aspects, N₂ is A. In some aspects, N₂ is C. In some aspects, N₂ is G. In some aspects, N₂ is U.

In some aspects, a second nucleotide in a Cap 1 structure may comprise one or more modifications, e.g., methylation. In some aspects, a Cap 1 structure comprising a second nucleotide comprising a 2′O methylation is a Cap 2 structure.

In some aspects, the RNA molecule may be enzymatically capped at the 5′ end using Vaccinia guanylyltransferase, guanosine triphosphate, and S-adenosyl-L-methionine to yield Cap 0 structure. An inverted 7-methylguanosine cap is added via a 5′ to 5′ triphosphate bridge. Alternatively, use of a 2′O-methyltransferase with Vaccinia guanylyltransferase yields the Cap 1 structure where in addition to the Cap 0 structure, the 2′OH group is methylated on the penultimate nucleotide. S-adenosyl-L-methionine (SAM) is a cofactor utilized as a methyl transfer reagent. Non-limiting examples of 5′ cap structures are those which, among other things, have enhanced binding of cap binding polypeptides, increased half-life, reduced susceptibility to 5′ endonucleases and/or reduced 5′ decapping, as compared to synthetic 5′ cap structures known in the art (or to a wild type, natural or physiological 5′ cap structure). For example, recombinant Vaccinia Virus Capping Enzyme and recombinant 2′ O-methyltransferase enzyme may create a canonical 5′-5′-triphosphate linkage between the 5′-terminal nucleotide of an mRNA and a guanine cap nucleotide wherein the cap guanine includes an N7 methylation and the 5′-terminal nucleotide of the mRNA includes a 2′-O-methyl. Such a structure is termed the Cap 1 structure. This cap results in a higher translational-competency and cellular stability and a reduced activation of cellular pro-inflammatory cytokines, as compared, e.g., to other 5′ cap analog structures known in the art.

In some aspects, the 5′ terminal cap includes a cap analog, for example, a 5′ terminal cap may include a guanine analog. Exemplary guanine analogs include, but are not limited to, inosine, N1-methyl-guanosine, 2′-fluoro-guanosine, 7-deaza-guanosine, 8-oxo-guanosine, 2-amino-guanosine, LNA-guanosine, and 2-azido-guanosine.

In some aspects, the capping region may include a single cap or a series of nucleotides forming the cap. In this aspect the capping region may be from 1 to 10, e.g. 2-9, 3-8, 4-7, 1-5, 5-10, or at least 2, or 10 or fewer nucleotides in length. In this aspect the capping region is at least, at most, exactly, or between any two of 1, 2, 3, 4, 5, 6, 7, 8, 9, or 10 nucleotides in length. In some aspects, the cap is absent. In some aspects, the first and second operational regions may range from 3 to 40, e.g., 5-30, 10-20, 15, or at least 4, or 30 or fewer nucleotides in length and may comprise, in addition to a Start and/or Stop codon, one or more signal and/or restriction sequences. In some aspects, the first and second operational regions are at least, at most, exactly, or between any two of 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 nucleotides in length and may comprise, in addition to a Start and/or Stop codon, one or more signal and/or restriction sequences.

Further examples of 5′ cap structures include, but are not limited to, glyceryl, inverted deoxy abasic residue (moiety), 4′, 5′ methylene nucleotide, 1-(beta-D-erythrofuranosyl) nucleotide, 4′-thio nucleotide, carbocyclic nucleotide, 1,5-anhydrohexitol nucleotide, L-nucleotides, alpha-nucleotide, modified base nucleotide, threo-pentofuranosyl nucleotide, acyclic 3′,4′-seco nucleotide, acyclic 3,4-dihydroxybutyl nucleotide, acyclic 3,5 dihydroxypentyl nucleotide, 3′-3′-inverted nucleotide moiety, 3′-3′-inverted abasic moiety, 3′-2′-inverted nucleotide moiety, 3′-2′-inverted abasic moiety, 1,4-butanediol phosphate, 3′-phosphoramidate, hexylphosphate, aminohexyl phosphate, 3′-phosphate, 3′phosphorothioate, phosphorodithioate, or bridging or non-bridging methylphosphonate moiety. In some aspects, the RNA molecule of the present disclosure comprises at least one 5′ cap structure. In some aspects, the RNA molecule of the present disclosure does not comprise a 5′ cap structure.

In one aspect, the 5′ capping structure comprises a modified 5′ Cap 1 structure (m⁷G⁺m^{3′}-5′-ppp-5′-Am). In one aspect, the 5′ capping structure comprises is (3′OMe)-m₂^{7,3′-O}Gppp (m₁^{2′O})ApG (Trilink). This molecule is identical to the natural RNA cap structure in that it starts with a guanosine methylated at N7, and is linked by a 5′ to 5′ triphosphate linkage to the first coded nucleotide of the transcribed RNA (in this case, an adenosine). This guanosine is also methylated at the 3′ hydroxyl of the ribose to mitigate possible reverse incorporation of the cap molecule. The 2′ hydroxyl of the ribose on the adenosine is methylated, conferring a Cap1 structure.

### 3. Untranslated Regions (UTRS)

The 5′ UTR is a regulatory region situated at the 5′ end of a protein open reading frame that is transcribed into mRNA but not translated into an amino acid sequence or to the corresponding region in an RNA polynucleotide, such as an mRNA molecule. An untranslated region (UTR) may be present 5′ (upstream) of an open reading frame (5′ UTR) and/or 3′ (downstream) of an open reading frame (3′ UTR).

In some aspects, the UTR is derived from an mRNA that is naturally abundant in a specific tissue (e.g., lymphoid tissue), to which the mRNA expression is targeted. In some aspects, the UTR increases protein synthesis. Without being bound by mechanism or theory, the UTR may increase protein synthesis by increasing the time that the mRNA remains in translating polysomes (message stability) and/or the rate at which ribosomes initiate translation on the message (message translation efficiency). Accordingly, the UTR sequence may prolong protein synthesis in a tissue-specific manner.

In some aspects, the 5′ UTR and the 3′ UTR sequences are computationally derived. In some aspects, the 5′ UTR and the 3′ UTRs are derived from a naturally abundant mRNA in a tissue. The tissue may be, for example, liver, a stem cell or lymphoid tissue. The lymphoid tissue may include, for example, any one of a lymphocyte (e.g., a B-lymphocyte, a helper T-lymphocyte, a cytotoxic T-lymphocyte, a regulatory T-lymphocyte, or a natural killer cell), a macrophage, a monocyte, a dendritic cell, a neutrophil, an eosinophil and a reticulocyte. In some aspects, the 5′ UTR and the 3′ UTR are derived from an alphavirus. In some aspects, the 5′ UTR and the 3′ UTR are from a wild type alphavirus.

### 4. 5′ UTRS

In some aspects, an RNA disclosed herein comprises a 5′ UTR. A 5′ UTR, if present, is located at the 5′ end and starts with the transcriptional start site upstream of the start codon of a protein encoding region. A 5′ UTR is downstream of the 5′ cap (if present), e.g. directly adjacent to the 5′ cap. The 5′ UTR may contain various regulatory elements, e.g., 5′ cap structure, stem-loop structure, and an internal ribosome entry site (IRES), which may play a role in the control of translation initiation.

In some aspects, a 5′ UTR disclosed herein comprises a cap proximal sequence, e.g., as disclosed herein. In some aspects, a cap proximal sequence comprises a sequence adjacent to a 5′ cap. In some aspects, a cap proximal sequence comprises nucleotides in positions +1, +2, +3, +4, and/or +5 of an RNA polynucleotide.

In some aspects, a Cap structure comprises one or more polynucleotides of a cap proximal sequence. In some aspects, a Cap structure comprises an m7 Guanosine cap and nucleotide +1 (N₁) of an RNA polynucleotide. In some aspects, a Cap structure comprises an m7 Guanosine cap and nucleotide +2 (N₂) of an RNA polynucleotide. In some aspects, a Cap structure comprises an m7 Guanosine cap and nucleotides +1 and +2 (N₁ and N₂) of an RNA polynucleotide.

Those skilled in the art, reading the present disclosure, will appreciate that, in some aspects, one or more residues of a cap proximal sequence (e.g., one or more of residues +1, +2, +3, +4, and/or +5) may be included in an RNA by virtue of having been included in a cap entity that (e.g., a Cap 1 structure, etc); alternatively, in some aspects, at least some of the residues in a cap proximal sequence may be enzymatically added (e.g., by a polymerase such as a T7 polymerase). For example, in certain exemplified aspects where a (m₂^{7,3′-O})Gppp(m^{2′-O})ApG cap is utilized, +1 and +2 residues are the (m₂^{7,3′-O}) A and G residues of the cap, and +3, +4, and +5 residues are added by polymerase (e.g., T7 polymerase).

In preferred embodiments, the nucleic acid comprises at least one heterologous 5′-UTR, wherein the at least one heterologous 5′-UTR comprises a nucleic acid sequence derived from a 5′-UTR of gene selected from any one of HSD17B4, RPL32, ASAH1, ATP5A1, MP68, NDUFA4, NOSIP, RPL31, SLC7A3, TUBB4B, and UBQLN2, or from a homolog, a fragment or variant of any one of these genes.

In one aspect, an RNA disclosed herein comprises a 5′ UTR comprising a sequence having at least, at most, exactly, or between any two of 99%, 98%, 97%, 96%, 95%, 90%, 85%, or 80% identity to the following sequence: GA4AGGCGGCGCAWGAGAGAAGCCCAGACCAAWWACCWACCCAAA. In another embodiment, the 5′ UTR comprises a sequence having at least, at most, exactly, or between any two of 99%, 98%, 97%, 96%, 95%, 90%, 85%, or 80% identity to the following sequence: GAAWAAAC ΨAGΨAΨΨCΨΨCΨGGΨCCCCA CAGACWCAGA GAGAACCCGC CACC.

### 5. 3′ UTRS

In some aspects, an RNA disclosed herein comprises a 3′ UTR. A 3′ UTR, if present, is situated downstream of a protein coding sequence open reading frame, e.g., downstream of the termination codon of a protein-encoding region. A 3′ UTR is typically the part of an mRNA which is located between the protein coding sequence and the poly-A tail of the mRNA. Thus, in some aspects, the 3′ UTR is upstream of the poly-A sequence (if present), e.g. directly adjacent to the poly-A sequence. The 3′ UTR may be involved in regulatory processes including transcript cleavage, stability and polyadenylation, translation, and mRNA localization.

A 3′ UTR may also comprise elements, which are not encoded in the template, from which an RNA is transcribed, but which are added after transcription during maturation, e.g. a poly-A tail. A 3′ UTR of the mRNA is not translated into an amino acid sequence. In some aspects, an RNA disclosed herein comprises a 3′ UTR comprising an F element and/or an I element. In some aspects, a 3′ UTR or a proximal sequence thereto comprises a restriction site. In some aspects, an RNA disclosed herein comprises a 3′ UTR comprising a sequence having at least, at most, exactly, or between any two of 99%, 98%, 97%, 96%, 95%, 90%, 85%, or 80% identity to CΨCGAGCΨGGΨACΨGCAΨGCA CGCAAΨGCΨA GCΨGCCCCΨΨΨCCCGΨCCΨG GGΨACCCCGA GΨCΨCCCCCG ACCΨCGGGΨC CCAGGΨAΨGC ΨCCCACCΨCC ACCΨGCCCCA CΨCACCACCΨCΨGCΨAGΨΨC CAGACACCΨC CCAAGCACGC AGCAAΨGCAG CΨCAAAACGC ΨΨAGCCΨAGC CACACCCCCA CGGGAAACAG CAGΨGAΨΨAA CCΨΨΨAGCAA ΨAAACGAAAG ΨΨΨAACΨAAG CΨAΨACΨAAC CCCAGGGΨΨG GΨCAAΨΨΨCG ΨGCCAGCCAC ACCCΨGGAGC ΨAGC. In preferred embodiments, the nucleic acid comprises at least one heterologous 3′-UTR, wherein the at least one heterologous 3′-UTR comprises a nucleic acid sequence derived from a 3′-UTR of a gene selected from PSMB3, ALB7, alpha-globin (referred to as “muag”), CASP1, COX6B1, GNAS, NDUFA1 and RPS9, or from a homolog, a fragment or variant of any one of these genes.

### 6. Poly-A Tail

In some aspects, RNA molecules disclosed herein comprise a poly-adenylate (poly-A) sequence. In some aspects, a poly-A sequence is situated downstream of a 3′ UTR, e.g., adjacent to a 3′ UTR. A “poly-A tail” or “poly-A sequence” refers to a stretch of consecutive adenine residues, which may be attached to the 3′ end of the RNA molecule. Poly-A sequences are known to those of skill in the art and may follow the 3′ UTR in the RNA molecules described herein. The poly-A tail may increase the half-life of the RNA molecule.

RNA molecules disclosed herein may have a poly-A sequence attached to the free 3′-end of the RNA by a template-independent RNA polymerase after transcription or a poly-A sequence encoded by DNA and transcribed by a template-dependent RNA polymerase. In some aspects, a poly-A sequence is attached during RNA transcription, e.g., during preparation of in vitro transcribed RNA, based on a DNA template comprising repeated dT nucleotides (deoxythymidylate) in the strand complementary to the coding strand.

In some aspects, the poly-A sequence contained in an RNA polynucleotide described herein essentially consists of adenosine nucleotides, but is interrupted by a random sequence of the four nucleotides (A, C, G, U). Such a random sequence may be at least, at most, exactly, or between any two of 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides in length.

In some aspects, no nucleotides other than adenosine nucleotides flank a poly-A sequence at its 3′-end, e.g., the poly-A sequence, is not masked or followed at its 3′-end by a nucleotide other than adenosine.

The poly-A sequence may be of any length. In some aspects, the poly-A tail may include 5 to 300 nucleotides in length. In some aspects, the RNA molecule includes a poly-A tail that comprises, essentially consists of, or consists of a sequence of about 25 to about 400 adenosine nucleotides, a sequence of about 50 to about 400 adenosine nucleotides, a sequence of about 50 to about 300 adenosine nucleotides, a sequence of about 50 to about 250 adenosine nucleotides, a sequence of about 60 to about 250 adenosine nucleotides, or a sequence of about 40 to about 100 adenosine nucleotides. In some aspects, the poly-A tail comprises, essentially consists of, or consists of at least, at most, exactly, or between any two of 5, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 405, 410, 415, 420, 425, 430, 435, 440, 445, 450, 455, 460, 465, 470, 475, 480, 485, 490, 495, or 500 adenosine nucleotides. In this context, “essentially consists of” means that most nucleotides in the poly-A sequence, typically at least 75%, at least 80%, at least 85%, at least 90%, at least 95%, at least 96%, at least 97%, at least 98%, or at least 99% by number of nucleotides in the poly-A sequence are adenosine nucleotides, but permits that remaining nucleotides are nucleotides other than adenosine nucleotides, such as uridine, guanosine, or cytosine. In this context, “consists of” means that all nucleotides in the poly-A sequence, e.g., 100% by number of nucleotides in the poly-A sequence, are adenosine nucleotides.

In some aspects, the RNA molecule includes a poly-A tail that includes a sequence of greater than 30 adenosine nucleotides. In some aspects, the RNA molecule includes a poly-A tail that includes about 40 adenosine nucleotides. In some aspects, the RNA molecule includes a poly-A tail that includes about 80 adenosine nucleotides. In some aspects, the 3′ poly-A tail has a stretch of at least 10 consecutive adenosine residues and at most 300 consecutive adenosine residues. In some specific aspects, the RNA molecule includes about 40 consecutive adenosine residues. In some aspects, the RNA molecule includes about 80 consecutive adenosine residues. Poly-A tails may play key regulatory roles in enhancing translation efficiency and regulating the efficiency of mRNA quality control and degradation. Short sequences or hyperpolyadenylation may signal for RNA degradation. Some designs include a poly-A tails of about 40 adenosine nucleotides, about adenosine nucleotides.

H. Self-Amplifying RNA (saRNA)

In some aspects, the RNA molecule may be an saRNA. “Self-amplifying RNA,” “self-amplifying RNA,” “self-replicating” and “replicon” may all be used interchangeably, and refer to RNA with the ability to replicate itself. Self-amplifying RNA molecules may be produced by using replication elements derived from, e.g. alphaviruses, and substituting the structural viral polypeptides with a nucleotide sequence encoding a polypeptide of interest. A self-amplifying RNA molecule is typically a positive-strand molecule that may be directly translated after delivery to a cell, and this translation provides an RNA-dependent RNA polymerase which then produces both antisense and sense transcripts from the delivered RNA. The delivered RNA leads to the production of multiple daughter RNA molecules. These daughter RNA molecules, as well as collinear subgenomic transcripts, may be translated themselves to provide in situ expression of an encoded gene of interest, e.g., a viral antigen, or may be transcribed to provide further transcripts with the same sense as the delivered RNA which are translated to provide in situ expression of the antigen. The overall result of this sequence of transcriptions is an amplification in the number of the introduced saRNA molecules and so the encoded gene of interest, e.g., a viral antigen, becomes a major polypeptide product of the cells.

In some aspects, the self-amplifying RNA includes at least one or more genes including any one of viral replicases, viral proteases, viral helicases and other nonstructural viral proteins, or combination thereof. In some aspects, the self-amplifying RNA may also include 5′- and 3′-end tractive replication sequences, and optionally a heterologous sequence that encodes a desired amino acid sequence (e.g., an antigen of interest). A subgenomic promoter that directs expression of the heterologous sequence may be included in the self-amplifying RNA. Optionally, the heterologous sequence (e.g., an antigen of interest) may be fused in frame to other coding regions in the self-amplifying RNA and/or may be under the control of an internal ribosome entry site (IRES).

In some aspects, a self-amplifying RNA molecule described herein encodes (i) an RNA-dependent RNA polymerase that may transcribe RNA from the self-amplifying RNA molecule and (ii) a polypeptide of interest, e.g., a viral antigen. In some aspects, the polymerase may be an alphavirus replicase, e.g., including any one of alphavirus protein nsP1, nsP2, nsP3, nsP4, and any combination thereof.

### I. RNA Encapsulation

The RNA in an RNA product solution may be encapsulated, and the RNA solution may further comprise at least one encapsulating agent. In one aspect, the encapsulating agent comprises a lipid, a lipid nanoparticle (LNP), lipoplexes, polymeric particles, polyplexes, and monolithic delivery systems, and a combination thereof. In some aspects, 1, 2, 3, 4, 5, or more of the foregoing elements may be excluded as an encapsulating agent.

In some aspects, LNPs may be designed to protect RNA molecules (e.g., saRNA, mRNA) from extracellular RNases and/or may be engineered for systemic delivery of the RNA to target cells. In some aspects, such LNPs may be particularly useful to deliver RNA molecules (e.g., mRNA, saRNA, modRNA) when RNA molecules are intravenously administered to a subject in need thereof. In some aspects, such LNPs may be particularly useful to deliver RNA molecules (e.g., saRNA, mRNA) when RNA molecules are intramuscularly administered to a subject in need thereof.

In one aspect, the RNA in the RNA solution is at a concentration of <1 mg/mL. In another aspect, the RNA is at a concentration of at least about 0.05 mg/mL. In another aspect, the RNA is at a concentration of at least about 0.5 mg/mL. In another aspect, the RNA is at a concentration of at least about 1 mg/mL. In another aspect, the RNA concentration is from about 0.05 mg/mL to about 0.5 mg/mL. In another aspect, the RNA is at a concentration of at least 10 mg/mL. In another aspect, the RNA is at a concentration of at least 50 mg/mL. In some aspects, the RNA is at a concentration of at least, at most, exactly, or between any two of about 0.05 mg/mL, 0.5 mg/mL, 1 mg/mL, 10 mg/mL, 50 mg/mL, 75 mg/mL, 100 mg/mL, 150 mg/mL, 200 mg/mL, 250 mg/mL, 300 mg/mL, 400 mg/mL, or more.

The present disclosure provides for an RNA solution and lipid preparation mixture or compositions thereof comprising at least one RNA encoding, e.g., an antigen (e.g., a hMPV F protein mutant and/or an antigen derived from PIV1 and/or an antigen derived from PIV3) complexed with, encapsulated in, and/or formulated with one or more lipids, and forming lipid nanoparticles (LNPs), liposomes, lipoplexes and/or nanoliposomes. In some aspects, the composition comprises a lipid nanoparticle.

Preferably, the LNP comprises (i) at least one cationic lipid; (ii) at least one neutral lipid; (iii) at least one steroid or steroid analogue, preferably cholesterol; and (iv) at least one polymer conjugated lipid, preferably a PEG-lipid; wherein (i) to (iv) are in a molar ratio of about 20-60% cationic lipid, 5-25% neutral lipid, 25-55% sterol, and 0.5-15% polymer conjugated lipid.

In some aspects, the lipid nanoparticles comprise one or more cationic lipids. In one aspect, the lipid nanoparticles comprise (4-hydroxybutyl)azanediyl)bis(hexane-6,1-diyl)bis(2-hexyldecanoate) (ALC-0315), having the formula:

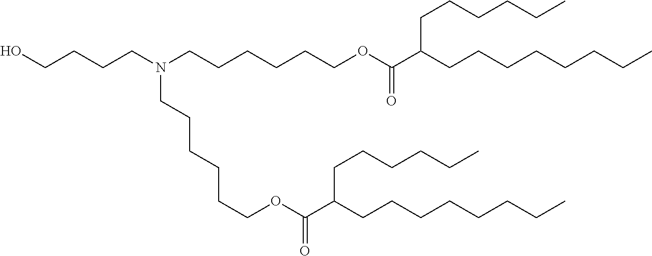 [SMILES: CCCCCCCCC(CCCCCC)C(=O)OCCCCCCN(CCCCO)CCCCCCOC(=O)C(CCCCCC)CCCCCCCC]

In embodiments, the cationic lipid is present in the LNP in an amount from about 30 to about 70 mole percent. In one embodiment, the cationic lipid is present in the LNP in an amount from about 40 to about 60 mole percent, such as about 40, 41, 42, 43, 44, 45, 46, 47, 48, 49, 50, 51, 52, 53, 54, 55, 56, 57, 58, 59 or 60 mole percent, respectively. In embodiments, the cationic lipid is present in the LNP in an amount from about 47 to about 48 mole percent, such as about 47.0, 47.1, 47.2, 47.3, 47.4, 47.5, 47.6, 47.7, 47.8, 47.9, 50.0 mole percent, respectively, wherein 47.7 mole percent are particularly preferred.

In some embodiments, the cationic lipid is present in a ratio of from about 20 mol % to about 70 or 75 mol % or from about 45 to about 65 mol % or about 20, 25, 30, 35, 40, 45, 50, 55, 60, 65, or about 70 mol % of the total lipid present in the LNP. In further embodiments, the LNPs comprise from about 25% to about 75% on a molar basis of cationic lipid, e.g., from about 20 to about 70%, from about 35 to about 65%, from about 45 to about 65%, about 60%, about 57.5%, about 57.1%, about 50% or about 40% on a molar basis (based upon 100% total moles of lipid in the lipid nanoparticle). In some embodiments, the ratio of cationic lipid to nucleic acid (e.g. coding RNA or DNA) is from about 3 to about 15, such as from about 5 to about 13 or from about 7 to about 11.

In some aspects, the LNPs comprise a polymer conjugated lipid. The term “polymer conjugated lipid” refers to a molecule comprising both a lipid portion and a polymer portion. An example of a polymer conjugated lipid is a pegylated lipid. The term “pegylated lipid” refers to a molecule comprising both a lipid portion and a polyethylene glycol portion. Pegylated lipids are known in the art and include 1-(monomethoxy-polyethyleneglycol)-2,3-dimyristoylglycerol (PEG-s-DMG), 2-[(polyethylene glycol)-2000]-N,N-ditetradecylacetamide, and the like.

In some aspects, the lipid nanoparticles comprise a polymer conjugated lipid. In one aspect, the lipid nanoparticle comprises 2-[(polyethylene glycol)-2000]-N,N-ditetradecylacetamide (ALC-0159), having the formula:

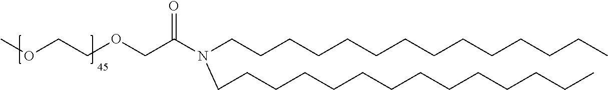

In various aspects, the molar ratio of the cationic lipid to the pegylated lipid ranges from about 100:1 to about 20:1, e.g., from about 20:1, 25:1, 30:1, 35:1, 40:1, 45:1, 50:1, 55:1, 60:1, 65:1, 70:1, 75:1, 80:1, 85:1, 90:1, 95:1, or 100:1, or any range or value derivable therein.

In certain aspects, the PEG-lipid is present in the LNP in an amount from about 1 to about 10 mole percent (mol %) (e.g., at least, at most, exactly, or between any two of 1, 2, 3, 4, 5, 6, 7, 8, 9, or 10 mol %), relative to the total lipid content of the nanoparticle. In some embodiments, LNPs include less than about 3, 2, or 1 mole percent of PEG or PEG-modified lipid, based on the total moles of lipid in the LNP. In further embodiments, LNPs comprise from about 0.1% to about 20% of the PEG-modified lipid on a molar basis, e.g., about 0.5 to about 10%, about 0.5 to about 5%, about 10%, about 5%, about 3.5%, about 3%, about 2.5%, about 2%, about 1.5%, about 1%, about 0.5%, or about 0.3% on a molar basis (based on 100% total moles of lipids in the LNP). In preferred embodiments, LNPs comprise from about 1.0% to about 2.0% of the PEG-modified lipid on a molar basis, e.g., about 1.2 to about 1.9%, about 1.2 to about 1.8%, about 1.3 to about 1.8%, about 1.4 to about 1.8%, about 1.5 to about 1.8%, about 1.6 to about 1.8%, in particular about 1.4%, about 1.5%, about 1.6%, about 1.7%, about 1.8%, about 1.9%, most preferably 1.7% (based on 100% total moles of lipids in the LNP).

In some aspects, provided RNA molecules (e.g., mRNA, saRNA, modRNA) may be formulated with LNPs. In some aspects, the lipid nanoparticles may have a mean diameter of about 1 to 500 nm. In some aspects, the lipid nanoparticles have a mean diameter of from about 30 nm to about 150 nm, from about 40 nm to about 150 nm, from about 50 nm to about 150 nm, from about 60 nm to about 130 nm, from about 70 nm to about 110 nm, from about 70 nm to about 100 nm, from about 80 nm to about 100 nm, from about 90 nm to about 100 nm, from about 70 to about 90 nm, from about 80 nm to about 90 nm, from about 70 nm to about 80 nm, or at least, at most, exactly, or between any two of 30 nm, 35 nm, 40 nm, 45 nm, 50 nm, 55 nm, 60 nm, 65 nm, 70 nm, 75 nm, 80 nm, 85 nm, 90 nm, 95 nm, 100 nm, 105 nm, 110 nm, 115 nm, 120 nm, 125 nm, 130 nm, 135 nm, 140 nm, 145 nm, or 150 nm, and are substantially non-toxic. The term “mean diameter” refers to the mean hydrodynamic diameter of particles as measured by dynamic laser light scattering (DLS) with data analysis using the so-called cumulant algorithm, which provides as results the so-called Z-average with the dimension of a length, and the polydispersity index (PI), which is dimensionless (Koppel, D., J. Chem. Phys. 57, 1972, pp 4814-4820, ISO 13321). Here, “mean diameter,” “diameter,” or “size” for particles is used synonymously with this value of the Z-average.

LNPs described herein may exhibit a polydispersity index less than about 0.5, less than about 0.4, less than about 0.3, or about 0.2 or less. By way of example, the LNPs may exhibit a polydispersity index of at least, at most, exactly, or between any two of 0.1, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, 0.3, 0.31, 0.32, 0.33, 0.34, 0.35, 0.36, 0.37, 0.38, 0.39, 0.4, 0.41, 0.42, 0.43, 0.44, 0.45, 0.46, 0.47, 0.48, 0.49, or 0.5. The polydispersity index is, in some aspects, calculated based on dynamic light scattering measurements by the so-called cumulant analysis as mentioned in the definition of the “average diameter.” Under certain prerequisites, it may be taken as a measure of the size distribution of an ensemble of nanoparticles.

In certain aspects, nucleic acids, when present in the lipid nanoparticles, are resistant in aqueous solution to degradation with a nuclease. Lipid nanoparticles comprising nucleic acids and their method of preparation are disclosed in, e.g., U.S. Patent Publication Nos. 2004/0142025, 2007/0042031 and PCT Pub. Nos. WO 2013/016058 and WO 2013/086373, the full disclosures of which are herein incorporated by reference in their entirety for all purposes.

### J. ANTI-hMPV A and PIV1 F Protein Antibodies

The present disclosure relates to antibodies that specifically bind to one of hMPV and PIV1. The present invention also pertains to related molecules, e.g. nucleic acids which encode such antibodies, compositions, and related methods, e.g., methods for producing and purifying such antibodies, and their use in diagnostics and therapeutics

### 1. EXEMPLARY EMBODIMENTS (E) OF THE INVENTION

E1. An isolated antibody that binds to human metapneumovirus (hMPV), comprising a heavy chain variable region (hMPV-VH) and a light chain variable region (hMPV-VL), comprising the CDR-H1, CDR-H2, and CDR-H3 sequences of SEQ ID NO: 360, and the CDR-L1, CDR-L2, and CDR-L3 sequences of SEQ ID NO: 361;
E2. An isolated antibody that binds to hMPV, comprising a heavy chain variable region (hMPV-VH) and a light chain variable region (hMPV-VL), comprising a CDR-H1 sequence according to SEQ ID NO: 523 or 524; a CDR-H2 sequence according to SEQ ID NO: 525 or 526; a CDR-H3 sequence according to SEQ ID NO: 527 or 528 and comprising a CDR-L1 sequence according to SEQ ID NO: 529; a CDR-L2 sequence according to SEQ ID NO: 530, and a CDR-L3 sequence according to SEQ ID NO: 531.
E2. An isolated antibody that binds to hMPV, comprising a heavy chain variable region (hMPV-VH) and a light chain variable region (hMPV-VL), comprising a CDR-H1 sequence according to SEQ ID NO: 523; a CDR-H2 sequence according to SEQ ID NO: 525; a CDR-H3 sequence according to SEQ ID NO: 527 and comprising a CDR-L1 sequence according to SEQ ID NO: 529; a CDR-L2 sequence according to SEQ ID NO: 530, and a CDR-L3 sequence according to SEQ ID NO: 531.
E3. An isolated antibody that binds to hMPV, comprising a heavy chain variable region (hMPV-VH) and a light chain variable region (hMPV-VL), comprising a CDR-H1 sequence according to SEQ ID NO: 524; a CDR-H2 sequence according to SEQ ID NO: 526; a CDR-H3 sequence according to SEQ ID NO: 528 and comprising a CDR-L1 sequence according to SEQ ID NO: 529; a CDR-L2 sequence according to SEQ ID NO: 530, and a CDR-L3 sequence according to SEQ ID NO: 531.
E4. The antibody of any one of E1-E3, comprising a hMPV-VH sequence at least 90%, 91%, 92%, 93%, 94%, 95%, 96%, 97%, 98%, 99%, or 100% identical to SEQ ID NO: 360, and comprising a hMPV-VL sequence at least 90%, 91%, 92%, 93%, 94%, 95%, 96%, 97%, 98%, 99%, or 100% identical to SEQ ID NO: 361.
E5. The antibody of any one of E1-E4, comprising a hMPV-VH sequence of SEQ ID NO: 360, and comprising a hMPV-VL sequence of SEQ ID NO: 361.
E6. The antibody of any one of E1-E5, comprising a hMPV-VH sequence encoded by a polynucleotide sequence of SEQ ID NO: 545.
E7. The antibody of any one of E1-E6, comprising a hMPV-VL sequence encoded by a nucleic acid sequence of SEQ ID NO 546.
E8. An isolated antibody comprising an hMPV-VH sequence encoded by a nucleic acid sequence of SEQ ID NO: 545 and comprising a, hMPV-VL sequence encoded by a nucleic acid sequence of SEQ ID NO 546.
E9. The antibody of any one of E1-E8, further comprising an Fc domain, wherein the Fc domain is an isotype of IgA (for example IgA₁ or IgA₂), IgD, IgE, IgM, or IgG (for example IgG₁, IgG₂, IgG₃, or IgG₄).
E10. The antibody of any one of E1-E9, comprising a Fc domain of the isotype of IgG.
E11. The antibody of any one of E1-E10, comprising a Fc domain of an isotype of IgG₁.
E12. The antibody of any one of E1-E11, comprising a heavy chain having the amino acid sequence of SEQ ID NO: 532
E13. The antibody of any one of E1-E12, comprising a light chain having the amino acid sequence of SEQ ID NO: 533.
E14. The antibody of any one of E1-E13, comprising a heavy chain having the amino acid sequence of SEQ ID NO: 532, and a light chain having the amino acid sequence of SEQ ID NO: 533.
E15. The antibody of any one of E1-E14, wherein the antibody binds to hMPV F protein.
E16. The antibody of any one of E1-E15, wherein the antibody binds to hMPV A.
E17. The antibody of any one of E1-E16, wherein the antibody binds to hMPV B.
E18 The antibody of any one of E1-E17, wherein the antibody binds to hMPV A and B.
E19. The antibody of any one of E1-E18, wherein the antibody specifically binds to hMPV F protein in prefusion conformation.
E20. The antibody of E19, wherein the antibody specifically binds to hMPV A F protein in prefusion conformation.
E21. The antibody of E19, wherein the antibody specifically binds to hMPV B F protein in prefusion conformation.
E22. The antibody of E19 to E21, wherein the binding KD of the antibody to the prefusion conformation is at least 100 orders of magnitude higher than the binding KD of the antibody to the post fusion conformation, as measured by SPR.
E23. The antibody of E19 to E21, wherein the binding KD of the antibody to the prefusion specific form is at least 1000 orders of magnitude higher than the binding KD of the antibody to the post fusion specific form, as measured by SPR.
E24. The antibody of any one of E1-E23, wherein the antibody neutralizes hMPV.
E25. The antibody of any one of E1-E24, wherein the antibody neutralizes hMPV A.
E26. The antibody of any one of E1-E25, wherein the antibody neutralizes hMPV B.
E27. The antibody of any one of E1-E26, wherein the antibody is characterised by an IC50 of less than 100 μg/ml as determined by plaque reduction neutralization test.
E28. The antibody of any one of E1-E27 wherein the antibody is characterised by an IC50 of less than 50 μg/ml as determined by plaque reduction neutralization test.
E29. The antibody of any one of E1-E27, wherein the antibody is characterised by an IC50 of between 100 μg/ml and 1 μg/ml as determined by plaque reduction neutralization test.
E30. The antibody of any one of E1-E27, wherein the antibody is characterised by an IC50 of about 20 μg/ml as determined by plaque reduction neutralization test.
E31. An isolated antibody that that competes for binding to hMPV with a second antibody comprising a VH having the amino acid sequence of SEQ ID NO: 360, and a VL having the amino acid sequence of SEQ ID NO: 361.
E32. A pharmaceutical composition comprising a therapeutically effective amount of the antibody of any one of E1-E31 and a pharmaceutically acceptable carrier.
E33. An isolated polynucleotide encoding the antibody of any one of E1 to E31.
E34. The polynucleotide of E33, wherein said polynucleotide is RNA.
E35. The polynucleotide of E34, wherein said polynucleotide comprises at least one chemical modification.
E36. The polynucleotide of E35, wherein the chemical modification wherein is selected from pseudouridine, 1-methylpseudouridine. N1-methylpseudouridine, N1-ethylpseudouridine, 2-thiouridine, 4′-thiouridine, 5-methylcytosine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methyluridine,), 5-methoxyuridine and 2′-O-methyl uridine.
E37. The polynucleotide of E34, wherein said polynucleotide does not comprise a chemical modification.
E38. An isolated polynucleotide encoding the VH of an antibody that binds hMPV, wherein the polynucleotide comprises the nucleic acid sequence of SEQ ID NO 545.
E39. An isolated polynucleotide encoding the VL of an antibody that binds hMPV, wherein the polynucleotide comprises the nucleic acid sequence of SEQ ID NO: 546.
E40. An isolated polynucleotide encoding the VH and VL of an antibody that binds hMPV wherein the polynucleotide encoding the VH comprises the nucleic acid sequence of SEQ ID NO: 545 and the polynucleotide encoding the VL comprises the nucleic acid sequence of SEQ ID NO: 546.
E41. An isolated polynucleotide encoding the heavy chain, light chain, or both, of an antibody that binds hMPV, and wherein said nucleic acid comprises: the nucleic acid sequence of SEQ ID NO: 547 the nucleic acid sequence of SEQ ID NO: 548, or both.
E42. The isolated polynucleotide of any of E33 to E41, wherein the antibody binds hMPV A.
E43. The isolated polynucleotide of any of E33 to E41, wherein the antibody binds hMPV B.
E44. The isolated polynucleotide of any of E33 to E41, wherein the antibody binds hMPV A and B.
E45. The isolated polynucleotide of any of E33 to E44, wherein the antibody binds hMPV F protein.
E46. The isolated polynucleotide of any of E33 to E41, wherein the antibody binds hMPV A F protein.
E47. The isolated polynucleotide of any of E33 to E41, wherein the antibody binds hMPV B F protein.
E48. The isolated polynucleotide of any of E33 to E41, wherein the antibody specifically binds to hMPV F protein in prefusion conformation.
E49. The polynucleotide of any of E33 to E41, wherein the antibody specifically binds to hMPV A F protein in prefusion conformation.
E50. A vector comprising the polynucleotide of any one of E33 to E49.
E51. An isolated host cell comprising the polynucleotide of any one of E33 to E49, or the vector of E50.
E52. A method of producing an isolated antibody, comprising culturing the host cell of E51 under conditions that result in production of the antibody, and recovering the antibody.
E53. The antibody of any one of E1-E31, or the pharmaceutical composition of E32, for use as a medicament.
E54. The antibody of any one of E1-E31, or the pharmaceutical composition of E32, for use in the treatment of a respiratory virus infection.
E55. The antibody of any one of E1-E31, or the pharmaceutical composition of E32, for use in the treatment of hMPV infection.
E56. The antibody of any one of E1-E31, or the pharmaceutical composition of E32, for use in the treatment of hMPV A infection.
E57. The antibody of any one of E1-E31, or the pharmaceutical composition of E32, for use in the treatment of hMPV B infection.
E58. A method of treating a medical condition, comprising administering to a subject in need thereof a therapeutically effective amount of the antibody of any one of E1-E31, or the pharmaceutical composition of E32.
E59. The method of E58, wherein the condition is a respiratory virus infection.
E60. The method of E58, wherein the condition is an hMPV infection.
E61. The method of any one of E58 to E60, or the use of E53 to E57, comprising administering said antibody, or pharmaceutical composition, subcutaneously.
E62. The method of any one of E58 to E60, or the use of E53 to E57 wherein said antibody, or pharmaceutical composition, is administered about twice a week, once a week, once every two weeks, once every three weeks, once every four weeks, once every five weeks, once every six weeks, once every seven weeks, once every eight weeks, once every nine weeks, once every ten weeks, twice a month, once a month, once every two months, once every three months, or once every four months.
E63. The use of the antibody of any one of E1-E31 for the manufacture of a medicament for use in the treatment of a respiratory virus infection.
E64. The use of the antibody of any one of E1-E31 for the manufacture of a medicament for use in the treatment of an hMPV infection.
E65. The use of the antibody of any one of E1-E31 for the manufacture of a medicament for use in the treatment of an hMPV A infection.
E66. An isolated antibody that binds to parainfluenza virus type 1 (PIV1), comprising a heavy chain variable region (PIV1-VH) and a light chain variable region (hMPV-VL), comprising the CDR-H1, CDR-H2, and CDR-H3 sequences of SEQ ID NO: 362, and the CDR-L1, CDR-L2, and CDR-L3 sequences of SEQ ID NO: 363;
E67. An isolated antibody that binds to PIV1, comprising a heavy chain variable region (PIV1-VH) and a light chain variable region (PIV1-VL), comprising a CDR-H1 sequence according to SEQ ID NO: 534 or 535; a CDR-H2 sequence according to SEQ ID NO: 536 or 537; a CDR-H3 sequence according to SEQ ID NO: 538 or 539 and comprising a CDR-L1 sequence according to SEQ ID NO: 540; a CDR-L2 sequence according to SEQ ID NO: 541, and a CDR-L3 sequence according to SEQ ID NO: 542.
E68. An isolated antibody that binds to PIV1, comprising a heavy chain variable region (PIV1-VH) and a light chain variable region (PIV1-VL), comprising a CDR-H1 sequence according to SEQ ID NO: 534; a CDR-H2 sequence according to SEQ ID NO: 536; a CDR-H3 sequence according to SEQ ID NO: 538 and comprising a CDR-L1 sequence according to SEQ ID NO: 540; a CDR-L2 sequence according to SEQ ID NO: 541, and a CDR-L3 sequence according to SEQ ID NO: 542.
E69. An isolated antibody that binds to PIV1, comprising a heavy chain variable region (PIV1-VH) and a light chain variable region (PIV1-VL), comprising a CDR-H1 sequence according to SEQ ID NO: 535; a CDR-H2 sequence according to SEQ ID NO: 537; a CDR-H3 sequence according to SEQ ID NO: 539 and comprising a CDR-L1 sequence according to SEQ ID NO: 540; a CDR-L2 sequence according to SEQ ID NO: 541, and a CDR-L3 sequence according to SEQ ID NO: 542.
E70. The antibody of any one of E66 to E69, comprising a PIV-VH sequence at least 90%, 91%, 92%, 93%, 94%, 95%, 96%, 97%, 98%, 99%, or 100% identical to SEQ ID NO: 362, and comprising a PIV1-VL sequence at least 90%, 91%, 92%, 93%, 94%, 95%, 96%, 97%, 98%, 99%, or 100% identical to SEQ ID NO: 363.
E71. The antibody of any one of E66 to E70, comprising a PIV1-VH sequence of SEQ ID NO: 362, and comprising a PIV1-VL sequence of SEQ ID NO: 363.
E72. The antibody of any one of E66 to E71, comprising a PIV1-VH sequence encoded by a polynucleotide sequence of SEQ ID NO: 549.
E73. The antibody of any one of E66 to E72, comprising a PIV1-VL sequence encoded by a nucleic acid sequence of SEQ ID NO 550.
E74 An isolated antibody comprising an PIV1-VH sequence encoded by a nucleic acid sequence of SEQ ID NO: 549 and comprising a, PIV1-VL sequence encoded by a nucleic acid sequence of SEQ ID NO 550.
E75. The antibody of any one of E66 to E74, further comprising an Fc domain, wherein the Fc domain is an isotype of IgA (for example IgA₁ or IgA₂), IgD, IgE, IgM, or IgG (for example IgG₁, IgG₂, IgG₃, or IgG₄).
E76. The antibody of any one of E66 to E75, comprising a Fc domain of the isotype of IgG.
E77. The antibody of any one of E66 to E76, comprising a Fc domain of an isotype of IgG₁.
E78. The antibody of any one of E66 to E77, comprising a heavy chain having the amino acid sequence of SEQ ID NO: 543
E79. The antibody of any one of E66 to E78, comprising a light chain having the amino acid sequence of SEQ ID NO: 544.
E80. The antibody of any one of E66 to E79, comprising a heavy chain having the amino acid sequence of SEQ ID NO: 543, and a light chain having the amino acid sequence of SEQ ID NO: 544.
E81. The antibody of any one of E66 to E80, wherein the antibody binds to PIV1 F protein.
E82. The antibody of any one of E66 to E81, wherein the antibody specifically binds to PIV1 F protein in prefusion conformation.
E83. The antibody of E82, wherein the binding KD of the antibody to the prefusion conformation is at least 100 orders of magnitude higher than the binding KD of the antibody to the post fusion conformation, as measured by SPR.
E84. The antibody of E82 or E83, wherein the binding KD of the antibody to the prefusion specific form is at least 1000 orders of magnitude higher than the binding KD of the antibody to the post fusion specific form, as measured by SPR.
E85. The antibody of any one of E66 to E84, wherein the antibody neutralizes PIV1.
E86. The antibody of any one of E66 to E85, wherein the antibody is characterised by an IC50 of less than 100 ng/ml as determined by plaque reduction neutralization test.
E87. The antibody of any one of E66 to E86, wherein the antibody is characterised by an IC50 of less than 10 ng/ml as determined by plaque reduction neutralization test.
E88. The antibody of any one of E66 to E85, wherein the antibody is characterised by an IC50 of between 100 ng/ml and 1 ng/ml as determined by plaque reduction neutralization test.
E89. The antibody of any one of E66 to E85, wherein the antibody is characterised by an IC50 of about 5 ng/ml as determined by plaque reduction neutralization test.
E90. An isolated antibody that that competes for binding to PIV1 with a second antibody comprising a VH having the amino acid sequence of SEQ ID NO: 362, and a VL having the amino acid sequence of SEQ ID NO: 363.
E91. A pharmaceutical composition comprising a therapeutically effective amount of the antibody of any one of E66 to E90 and a pharmaceutically acceptable carrier.
E92. An isolated polynucleotide encoding the antibody of any one of E66 to E90.
E93. The polynucleotide of E92, wherein said polynucleotide is RNA.
E94. The polynucleotide of E93, wherein said polynucleotide comprises at least one chemical modification.
E95. The polynucleotide of E94, wherein the chemical modification wherein is selected from pseudouridine, 1-methylpseudouridine. N1-methylpseudouridine, N1-ethylpseudouridine, 2-thiouridine, 4′-thiouridine, 5-methylcytosine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methyluridine,), 5-methoxyuridine and 2′-O-methyl uridine.
E96. The polynucleotide of E93, wherein said polynucleotide does not comprise a chemical modification.
E97. An isolated polynucleotide encoding the VH of an antibody that binds PIV1, wherein the polynucleotide comprises the nucleic acid sequence of SEQ ID NO 549.
E98. An isolated polynucleotide encoding the VL of an antibody that binds PIV1, wherein the polynucleotide comprises the nucleic acid sequence of SEQ ID NO: 550.
E99. An isolated polynucleotide encoding the VH and VL of an antibody that binds PIV1 wherein the polynucleotide encoding the VH comprises the nucleic acid sequence of SEQ ID NO: 549 and the polynucleotide encoding the VL comprises the nucleic acid sequence of SEQ ID NO: 550.
E100. An isolated polynucleotide encoding the heavy chain, light chain, or both, of an antibody that binds PIV1, and wherein said nucleic acid comprises: the nucleic acid sequence of SEQ ID NO: 551 the nucleic acid sequence of SEQ ID NO: 552, or both.
E101. A vector comprising the polynucleotide of any one of E92-E100.
E102. An isolated host cell comprising the polynucleotide of any one of E92-E100, or the vector of E101.
E103. A method of producing an isolated antibody, comprising culturing the host cell of E102 under conditions that result in production of the antibody, and recovering the antibody.
E104. The antibody of any one of E66 to E90, or the pharmaceutical composition of E91, for use as a medicament.
E105. The antibody of any one of E66 to E90, or the pharmaceutical composition of E91, for use in the treatment of a respiratory virus infection.
E106. The antibody of any one of E66 to E90, or the pharmaceutical composition of E91, for use in the treatment of PIV1 infection.
E107. A method of treating a medical condition, comprising administering to a subject in need thereof a therapeutically effective amount of the antibody of any one of E66 to E90, or the pharmaceutical composition of E91.
E108. The method of E107, wherein the condition is a respiratory virus infection.
E109. The method of E107, wherein the condition is a PIV1 infection.
E110. The method of any one of E107 to E109, or the use of E104 to E106 comprising administering said antibody, or pharmaceutical composition, subcutaneously.
E111. The method of any one of E107 to E109, or the use of E104 to E106 wherein said antibody, or pharmaceutical composition, is administered about twice a week, once a week, once every two weeks, once every three weeks, once every four weeks, once every five weeks, once every six weeks, once every seven weeks, once every eight weeks, once every nine weeks, once every ten weeks, twice a month, once a month, once every two months, once every three months, or once every four months.
E112. The use of the antibody of any one of E66 to E90 for the manufacture of a medicament for use in the treatment of a respiratory virus infection.
E113. The use of the antibody of any one of E66 to E90 for the manufacture of a medicament for use in the treatment of an PIV1 infection.
   2. Antibodies to hMPV
   The disclosure provides antibodies that bind to human metapneumovirus (hMPV). Human Metapneumovirus (hMPV) is as described herein.

In some embodiments, an antibody may be completely specific for hMPV and may not exhibit cross-reactivity with other viruses. In some embodiments, an antibody binds hMPV F protein. In some embodiments, an antibody may be completely specific for hMPV A F protein and may not exhibit cross-reactivity with other viruses. As used herein the term hMPV refers to naturally occurring human MPV unless contextually dictated otherwise. Therefore, an “hMPV antibody” “anti-hMPV antibody” or other similar designation means any antibody (as defined herein) that binds or reacts with hMPV, an isoform, fragment or derivative thereof. In one embodiment, the antibody hMPV A F protein. In one embodiment, the antibody binds hMPV B F protein. In one embodiment, the antibody binds hMPV A and B F protein. In some embodiments, an antibody may be specific for hMPV F protein in prefusion conformation.

In some embodiments, an anti-hMPV antibody of the disclosure encompasses an antibody that one or both of i) competes for binding to hMPV with or ii) binds the same epitope as, an antibody having the amino acid sequence of a heavy chain variable region set forth as SEQ ID NO:360 and the amino acid sequence of a light chain variable region set forth as SEQ ID NO: 361.

Anti-hMPV antibodies of the present disclosure can encompass monoclonal antibodies, polyclonal antibodies, antibody fragments (e.g., Fab, Fab′, F(ab′)₂, Fv, Fc, etc.), chimeric antibodies, bispecific antibodies, heteroconjugate antibodies, single chain (ScFv), mutants thereof, fusion proteins comprising an antibody fragment (e.g., a domain antibody), humanized antibodies, and any other modified configuration of the immunoglobulin molecule that comprises an antigen binding site of the required specificity, including glycosylation variants of antibodies, amino acid sequence variants of antibodies, and covalently modified antibodies. The antibodies may be murine, rat, human, or any other origin (including chimeric or humanized antibodies). In some embodiments, an anti-hMPV antibody is a monoclonal antibody. In some embodiments, an anti-hMPV antibody is a human or humanized antibody. In some embodiments, an anti-hMPV antibody is a chimeric antibody.

In some embodiments, the invention provides an antibody having a light chain variable region (VL) sequence and a heavy chain variable region (VH) sequence as found in Table 50, or variants thereof.

The invention also provides CDR portions of antibodies to hMPV. Determination of CDR regions is well within the skill of the art. It is understood that in some embodiments, CDRs can be a combination of the Kabat and Chothia CDR (also termed “combined CDRs” or “extended CDRs”). In another approach, referred to herein as the “conformational definition” of CDRs, the positions of the CDRs may be identified as the residues that make enthalpic contributions to antigen binding. See, e.g., Makabe et al., 2008, Journal of Biological Chemistry, 283:1156-1166. In general, “conformational CDRs” include the residue positions in the Kabat CDRs and Vernier zones which are constrained in order to maintain proper loop structure for the antibody to bind a specific antigen. Determination of conformational CDRs is well within the skill of the art. In some embodiments, the CDRs are the Kabat CDRs. In other embodiments, the CDRs are the Chothia CDRs. In other embodiments, the CDRs are the extended, AbM, conformational, or contact CDRs. In other words, in embodiments with more than one CDR, the CDRs may be any of Kabat, Chothia, extended, AbM, conformational, contact CDRs or combinations thereof.

Table 50 provides the CDR sequences of the anti-hMPV antibody provided herein.

In some embodiments, the antibody comprises one or both of i) the full-length heavy chain, with or without the C-terminal lysine, or ii) the full-length light chain of anti-hMPV antibody hMPV-2 mAb. The amino acid sequences of the full-length heavy chain and light chain for antibody hMPV-2 mAb is shown below in Table 50.

In some embodiments, the antibody that binds to hMPV, comprises a heavy chain variable region (hMPV-VH) and a light chain variable region (hMPV-VL), comprising the CDR-H1, CDR-H2, and CDR-H3 sequences of SEQ ID NO: 360, and the CDR-L1, CDR-L2, and CDR-L3 sequences of SEQ ID NO: 361.

In some embodiments, the antibody comprises a heavy chain variable region (hMPV-VH) and a light chain variable region (hMPV-VL), comprising a CDR-H1 sequence according to SEQ ID NO: 523 or 524; a CDR-H2 sequence according to SEQ ID NO: 525 or 526; a CDR-H3 sequence according to SEQ ID NO: 527 or 528 and comprising a CDR-L1 sequence according to SEQ ID NO: 529; a CDR-L2 sequence according to SEQ ID NO: 530, and a CDR-L3 sequence according to SEQ ID NO: 531.

In some embodiments, the antibody comprises a hMPV-VH sequence at least 90%, 91%, 92%, 93%, 94%, 95%, 96%, 97%, 98%, 99%, or 100% identical to SEQ ID NO: 360, and comprising a hMPV-VL sequence at least 90%, 91%, 92%, 93%, 94%, 95%, 96%, 97%, 98%, 99%, or 100% identical to SEQ ID NO: 361.

In some embodiments, the antibody comprises a hMPV-VH sequence of SEQ ID NO: 360 and comprising a hMPV-VL sequence of SEQ ID NO: 361.

In some embodiments, the antibody comprises a hMPV-VH sequence encoded by a nucleic acid sequence of SEQ ID NO: 547. In some embodiments, the antibody comprises a hMPV-VL sequence encoded by a nucleic acid sequence of SEQ ID NO: 548. In some embodiments, the antibody comprises a hMPV-VH sequence encoded by a nucleic acid sequence of SEQ ID NO: 547 and comprises a hMPV-VL sequence encoded by a nucleic acid sequence of SEQ ID NO: 548.

In some embodiments, the antibody comprises a heavy chain having the amino acid sequence of SEQ ID NO: 532. In some embodiments, the antibody comprises a light chain having the amino acid sequence of SEQ ID NO: 533. In some embodiments, the antibody comprises a heavy chain having the amino acid sequence of SEQ ID NO: 532, and a light chain having the amino acid sequence of SEQ ID NO: 533.

### 3. Antibodies to PIV1

The disclosure provides antibodies that bind to parainfluenza virus type 1 (PIV1). Parainfluenza virus type 1 (PIV1) is as described herein.

In some embodiments, an antibody may be completely specific for human PIV1 and may not exhibit cross-reactivity with other viruses. A “PIV1 antibody”, “anti-PIV1 antibody” or other similar designation means any antibody (as defined herein) that binds or reacts with PIV1, an isoform, fragment or derivative thereof. In some embodiments, an antibody may be specific for PIV1 F protein. In some embodiments, an antibody may be specific for PIV1 F protein. In some embodiments, an antibody may be specific for PIV1 F protein in prefusion conformation.

In some embodiments, an anti-PIV1 antibody of the disclosure encompasses an antibody that one or both of i) competes for binding to human PIV1 with or ii) binds the same epitope as, an antibody having the amino acid sequence of a heavy chain variable region set forth as SEQ ID NO: 362 and the amino acid sequence of a light chain variable region set forth as SEQ ID NO: 363.

Anti-PIV1 antibodies of the present disclosure can encompass monoclonal antibodies, polyclonal antibodies, antibody fragments (e.g., Fab, Fab′, F(ab′)₂, Fv, Fc, etc.), chimeric antibodies, bispecific antibodies, heteroconjugate antibodies, single chain (ScFv), mutants thereof, fusion proteins comprising an antibody fragment (e.g., a domain antibody), humanized antibodies, and any other modified configuration of the immunoglobulin molecule that comprises an antigen binding site of the required specificity, including glycosylation variants of antibodies, amino acid sequence variants of antibodies, and covalently modified antibodies. The antibodies may be murine, rat, human, or any other origin (including chimeric or humanized antibodies). In some embodiments, an anti-PIV1 antibody is a monoclonal antibody. In some embodiments, an anti-PIV1 antibody is a human or humanized antibody. In some embodiments, an anti-PIV1 antibody is a chimeric antibody.

In some embodiments, the invention provides an antibody having a light chain variable region (VL) sequence and a heavy chain variable region (VH) sequence as found in Table 50, or variants thereof.

The invention also provides CDR portions of antibodies to PIV1. Determination of CDR regions is well within the skill of the art. It is understood that in some embodiments, CDRs can be a combination of the Kabat and Chothia CDR (also termed “combined CDRs” or “extended CDRs”). In another approach, referred to herein as the “conformational definition” of CDRs, the positions of the CDRs may be identified as the residues that make enthalpic contributions to antigen binding. See, e.g., Makabe et al., 2008, Journal of Biological Chemistry, 283:1156-1166. In general, “conformational CDRs” include the residue positions in the Kabat CDRs and Vernier zones which are constrained in order to maintain proper loop structure for the antibody to bind a specific antigen. Determination of conformational CDRs is well within the skill of the art. In some embodiments, the CDRs are the Kabat CDRs. In other embodiments, the CDRs are the Chothia CDRs. In other embodiments, the CDRs are the extended, AbM, conformational, or contact CDRs. In other words, in embodiments with more than one CDR, the CDRs may be any of Kabat, Chothia, extended, AbM, conformational, contact CDRs or combinations thereof. Unless stated otherwise CDRs sequences herein are numbered by Kabat.

Table 50 provides examples of CDR sequences of anti-PIV1 antibodies provided herein.

In some embodiments, the antibody comprises one or both of i) the full-length heavy chain, with or without the C-terminal lysine, or ii) the full-length light chain of anti-PIV1 antibody PIV1-8 mAb. The amino acid sequences of the full-length heavy chain and light chain for antibody PIV1-8 mAb is shown below.

In some embodiments, the antibody that specifically binds to PIV1 F protein, comprises a heavy chain variable region (PIV1-VH) and a light chain variable region (PIV1-VL), comprising the CDR-H1, CDR-H2, and CDR-H3 sequences of SEQ ID NO: 362, and the CDR-L1, CDR-L2, and CDR-L3 sequences of SEQ ID NO: 363.

In some embodiments, the antibody comprises a heavy chain variable region (PIV1-VH) and a light chain variable region (PIV1-VL), comprising a CDR-H1 sequence according to SEQ ID NO: 534 or 535; a CDR-H2 sequence according to SEQ ID NO: 536 or 537; a CDR-H3 sequence according to SEQ ID NO: 538 or 539 and comprising a CDR-L1 sequence according to SEQ ID NO: 540; a CDR-L2 sequence according to SEQ ID NO: 541, and a CDR-L3 sequence according to SEQ ID NO: 542.

In some embodiments, the antibody comprises a PIV1-VH sequence at least 90%, 91%, 92%, 93%, 94%, 95%, 96%, 97%, 98%, 99%, or 100% identical to SEQ ID NO: 362, and comprising a PIV1-VL sequence at least 90%, 91%, 92%, 93%, 94%, 95%, 96%, 97%, 98%, 99%, or 100% identical to SEQ ID NO: 363.

In some embodiments, the antibody comprises a PIV1-VH sequence of SEQ ID NO: 362 and comprising a PIV1-VL sequence of SEQ ID NO: 363.

In some embodiments, the antibody comprises a PIV1-VH sequence encoded by a nucleic acid sequence of SEQ ID NO: 549. In some embodiments, the antibody comprises a PIV1-VL sequence encoded by a nucleic acid sequence of SEQ ID NO 550. In some embodiments, the antibody comprises a PIV1-VH sequence encoded by a nucleic acid sequence of SEQ ID NO: 549 and comprising a PIV1-VL sequence encoded by a nucleic acid sequence of SEQ ID NO 550.

In some embodiments, the antibody comprises a heavy chain having the amino acid sequence of SEQ ID NO: 543. In some embodiments, the antibody comprises a light chain having the amino acid sequence of SEQ ID NO: 544. In some embodiments, the antibody comprises a heavy chain having the amino acid sequence of SEQ ID NO: 543, and a light chain having the amino acid sequence of SEQ ID NO: 544.

### 4. Polynucleotides Encoding Antibodies of the Invention

The disclosure also provides polynucleotides encoding any of the antibodies of the invention, including antibody portions and modified antibodies described herein. The invention also provides a method of making any of the antibodies and polynucleotides described herein. Polynucleotides can be made and the proteins expressed by procedures known in the art.

If desired, an antibody (monoclonal or polyclonal) of interest may be sequenced and the polynucleotide sequence may then be cloned into a vector for expression or propagation. The sequence encoding the antibody of interest may be maintained in vector in a host cell and the host cell can then be expanded and frozen for future use. Production of recombinant monoclonal antibodies in cell culture can be carried out through cloning of antibody genes from B cells by means known in the art. See, e.g. Tiller et al., 2008, J. Immunol. Methods 329, 112; U.S. Pat. No. 7,314,622.

In some embodiments, provided herein is a polynucleotide comprising a sequence encoding one or both of the heavy chain or the light chain variable regions of an antibodies provided herein. The sequence encoding the antibody of interest may be maintained in a vector in a host cell and the host cell can then be expanded and frozen for future use. Vectors (including expression vectors) and host cells are further described herein.

In some embodiments, the disclosure provides a polynucleotide encoding the amino acid sequences of the PIV1 antibody listed in Table 50.

In one embodiment, the invention provides a polynucleotide encoding the amino acid sequence of the anti-hMPV antibody listed in Table 50.

In some embodiments, the disclosure provides a polynucleotide encoding an anti-hMPV antibody heavy chain polypeptide comprising an amino acid sequence: SEQ ID NO: 532. In some embodiments, the disclosure provides polynucleotide encoding an anti-hMPV antibody light chain polypeptide comprising an amino acid sequence of: SEQ ID NO: 533.

In some embodiments, the disclosure provides a polynucleotide encoding an anti-hMPV antibody VH polypeptide comprising an amino acid sequence of: SEQ ID NO: 360. In some embodiments, the disclosure provides a polynucleotide encoding an anti-hMPV antibody VL polypeptide comprising an amino acid sequence of: SEQ ID NOs: 361.

In some embodiments, the disclosure provides a polynucleotide encoding an anti-PIV1 antibody heavy chain polypeptide comprising an amino acid sequence of: SEQ ID NOs: 543. In some embodiments, the disclosure provides a polynucleotide encoding an anti-PIV1 antibody light chain polypeptide comprising an amino acid sequence of: SEQ ID NO: 544. In some embodiments, the disclosure provides a polynucleotide encoding an anti-PIV1 antibody VH polypeptide comprising an amino acid sequence of: SEQ ID NO: 362. In some embodiments, the disclosure provides a polynucleotide encoding an anti-PIV1 antibody VL polypeptide comprising an amino acid sequence of: SEQ ID NO: 363.

In some embodiments, the disclosure provides polynucleotides encoding the heavy chain, light chain, or both, of an antibody that binds hMPV, and wherein said polynucleotide comprise the nucleic acid sequence of SEQ ID NO: 547, the nucleic acid sequence of SEQ ID NO: 548, or both. In some embodiments, the disclosure provides polynucleotides encoding the heavy chain, light chain, or both, of an antibody that binds PIV1, and wherein said polynucleotide comprises the nucleic acid sequence of SEQ ID NO: 551, the nucleic acid sequence of SEQ ID NO: 552, or both.

In some embodiments, the disclosure provides polynucleotides encoding the variable heavy chain, variable light chain, or both, of an antibody that binds hMPV F protein, and wherein said polynucleotide comprise the nucleic acid sequence of SEQ ID NO: 545, the nucleic acid sequence of SEQ ID NO: 546, or both. In some embodiments, the disclosure provides polynucleotides encoding the variable heavy chain, variable light chain, or both, of an antibody that binds PIV1 F protein, and wherein said polynucleotide comprises the nucleic acid sequence of SEQ ID NO: 549, the nucleic acid sequence of SEQ ID NO: 550, or both.

It will be appreciated by those of ordinary skill in the art that, as a result of the degeneracy of the genetic code, there are many nucleotide sequences that encode a polypeptide as described herein. Some of these polynucleotides bear minimal homology to the nucleotide sequence of any native gene. Nonetheless, polynucleotides that vary due to differences in codon usage are specifically contemplated by the present invention. Further, alleles of the genes comprising the polynucleotide sequences provided herein are within the scope of the present invention. Alleles are endogenous genes that are altered as a result of one or more mutations, such as deletions, additions or substitutions of nucleotides. The resulting mRNA and protein may, but need not, have an altered structure or function. Alleles may be identified using standard techniques (such as hybridization, amplification or database sequence comparison).

In one embodiment, the VH and VL domains or full-length HC or LC, are encoded by separate polynucleotides. Alternatively, both VH and VL, or HC and LC, are encoded by a single polynucleotide.

Polynucleotides complementary to any such sequences are also encompassed by the present disclosure. Polynucleotides may be single-stranded (coding or antisense) or double-stranded, and may be DNA (genomic, cDNA or synthetic) or RNA molecules. RNA molecules include HnRNA molecules, which contain introns and correspond to a DNA molecule in a one-to-one manner, and mRNA molecules, which do not contain introns. Additional coding or non-coding sequences may, but need not, be present within a polynucleotide of the present disclosure, and a polynucleotide may, but need not, be linked to other molecules or support materials.

5. Methods of Manufacture of hMPV and PIV1 Antibodies

Various techniques for the production of antibodies have been described which include the traditional hybridoma method for making monoclonal antibodies, recombinant techniques for making antibodies (including chimeric antibodies, e.g., humanized antibodies), antibody production in transgenic animals and the recently described phage display technology for preparing “fully human” antibodies.

Provided herein are methods of making any of the antibodies provided herein. The antibodies of this invention can be made by procedures known in the art. The polypeptides can be produced by proteolytic or other degradation of the antibodies, by recombinant methods (e.g., single or fusion polypeptides) as described above or by chemical synthesis. Polypeptides of the antibodies, especially shorter polypeptides up to about 50 amino acids, are conveniently made by chemical synthesis. Methods of chemical synthesis are known in the art and are commercially available. For example, an antibody could be produced by an automated polypeptide synthesizer employing the solid phase method. See also, U.S. Pat. Nos. 5,807,715; 4,816,567; and 6,331,415.

The term “interface,” as used herein typically refers to any amino acid residue present in the domain that can be involved in first polypeptide and second polypeptide contacts. An “original amino acid” residue is one which is replaced by an “import amino acid” residue which can have a smaller or larger side chain volume than the original residue. The import amino acid residue can be a naturally occurring or non-naturally occurring amino acid residue, but preferably is the former. “Naturally occurring” amino acid residues are those residues encoded by the genetic code. By “non-naturally occurring” amino acid residue is meant a residue which is not encoded by the genetic code, but which is able to covalently bind adjacent amino acid residue(s) in the polypeptide chain. Examples of non-naturally occurring amino acid residues are norleucine, ornithine, norvaline, homoserine and other amino acid residue analogues such as those described in Ellman et al., Meth. Enzym. 202:301-336 (1991).

The polynucleotides encoding the antibodies of this invention can be obtained using chemical synthesis, recombinant methods, or PCR. Methods of chemical polynucleotide synthesis are well known in the art and need not be described in detail herein. One of skill in the art can use the sequences provided herein and a commercial DNA synthesizer to produce a desired DNA sequence.

For preparing polynucleotides using recombinant methods, a polynucleotide comprising a desired sequence can be inserted into a suitable vector, and the vector in turn can be introduced into a suitable host cell for replication and amplification, as further discussed herein. Polynucleotides may be inserted into host cells by any means known in the art. Cells are transformed by introducing an exogenous polynucleotide by direct uptake, endocytosis, transfection, F-mating or electroporation. Once introduced, the exogenous polynucleotide can be maintained within the cell as a non-integrated vector (such as a plasmid) or integrated into the host cell genome.

Suitable cloning vectors may be constructed according to standard techniques, or may be selected from a large number of cloning vectors available in the art. While the cloning vector selected may vary according to the host cell intended to be used, useful cloning vectors will generally have one or more features such as i) the ability to self-replicate, ii) a single target for a particular restriction endonuclease, or iii) may carry genes for a marker that can be used in selecting clones containing the vector. Suitable examples include plasmids and bacterial viruses, e.g., pUC18, pUC19, Bluescript (e.g., pBS SK+) and its derivatives, mp18, mp19, pBR322, pMB9, ColE1, pCR1, RP4, phage DNAs, and shuttle vectors such as pSA3 and pAT28. These and many other cloning vectors are available from commercial vendors such as BioRad, Strategene, and Invitrogen.

Expression vectors are further provided. Expression vectors generally are replicable polynucleotide constructs that contain a polynucleotide according to the invention. It is implied that an expression vector must be replicable in the host cells either as episomes or as an integral part of the chromosomal DNA. Suitable expression vectors include but are not limited to plasmids, viral vectors, including adenoviruses, adeno-associated viruses, retroviruses, cosmids, and expression vector(s) disclosed in PCT Publication No. WO 87/04462. Vector components may generally include, but are not limited to, one or more of the following: a signal sequence; an origin of replication; one or more marker genes; suitable transcriptional controlling elements (such as promoters, enhancers and terminator). For expression (e.g., translation), one or more translational controlling elements are also usually required, such as ribosome binding sites, translation initiation sites, and stop codons.

The vectors containing the polynucleotides of interest can be introduced into the host cell by any of a number of appropriate means, including electroporation, transfection employing calcium chloride, rubidium chloride, calcium phosphate, DEAE-dextran, or other substances; microprojectile bombardment; lipofection; and infection (e.g., where the vector is an infectious agent such as vaccinia virus). The choice of introducing vectors or polynucleotides will often depend on features of the host cell.

The invention also provides host cells comprising any of the polynucleotides described herein. Any host cells capable of over-expressing heterologous DNAs can be used for the purpose of isolating the genes encoding the antibody, polypeptide or protein of interest. Non-limiting examples of mammalian host cells include but not limited to COS, HeLa, and CHO cells. See also PCT Publication No. WO 87/04462. Suitable non-mammalian host cells include prokaryotes (such as E. coli or B. subtillis) and yeast (such as S. cerevisae, S. pombe; or K. lactis).

Additionally, any number of commercially and non-commercially available cell lines that express polypeptides or proteins may be utilized in accordance with the present invention. One skilled in the art will appreciate that different cell lines might have different nutrition requirements or might require different culture conditions for optimal growth and polypeptide or protein expression, and will be able to modify conditions as needed.

6. Pharmaceutical Compositions of hMPV and PIV1 ANtibodies

In another embodiment, the invention comprises pharmaceutical compositions of the hMPV and PIV1 antibodies.

A “pharmaceutical composition” refers to a mixture of an antibody the invention and one or excipient.

Pharmaceutical compositions of this invention may be in a variety of forms. These include, for example, liquid, semi-solid and solid dosage forms, such as liquid solutions (e.g., injectable and infusible solutions), dispersions or suspensions, and lyophilized powders. The form depends on the intended mode of administration and therapeutic application.

Other excipients and modes of administration known in the pharmaceutical art may also be used. Pharmaceutical compositions of the invention may be prepared by any of the well-known techniques of pharmacy, such as effective formulation and administration procedures. The above considerations in regard to effective formulations and administration procedures are well known in the art and are described in standard textbooks. Formulation of drugs is discussed in, for example, Hoover, John E., Remington's Pharmaceutical Sciences, Mack Publishing Co., Easton, Pennsylvania, 1975; Liberman et al., Eds., Pharmaceutical Dosage Forms, Marcel Decker, New York, N.Y., 1980; and Kibbe et al., Eds., Handbook of Pharmaceutical Excipients (3rd Ed.), American Pharmaceutical Association, Washington, 1999.

Acceptable excipients are nontoxic to recipients at the dosages and concentrations employed, and may comprise buffers such as phosphate, citrate, and other organic acids; salts such as sodium chloride; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens, such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); or non-ionic surfactants such as TWEEN™, PLURONICS™ or polyethylene glycol (PEG).

7. Therapeutic, Diagnostic and Other Methods of Using hMPV and PIV1 Antibodies

The antibodies of the present invention are useful in various applications including, but are not limited to, therapeutic treatment methods and diagnostic treatment methods.

In some embodiments, antibodies of the invention may neutralize hMPV. Antibodies of the invention may be useful in the treatment, prevention, suppression and amelioration of respiratory infections mediated by hMPV. In some embodiments, antibodies of the invention may neutralize PIV1. Antibodies of the invention may be useful in the treatment, prevention, suppression and amelioration of respiratory infections mediated by PIV1.

In one aspect, the invention provides a method for treating respiratory infections. In one aspect, the invention provides a method for treating hMPV infection. In one aspect, the invention provides a method for treating PIV1 infection. In some embodiments, the method of treating respiratory infections in a subject comprises administering to the subject in need thereof an effective amount of a pharmaceutical composition comprising any of the antibodies as described herein. In some embodiments, provided is a method of treating hMPV in a subject, comprising administering to the subject in need thereof an effective amount of a composition comprising an antibody provided herein. In some embodiments, provided is a method of treating PIV1 in a subject, comprising administering to the subject in need thereof an effective amount of a composition comprising an antibody provided herein.

In another aspect, the invention further provides an antibody or pharmaceutical composition as described herein for use in the described method of treating inflammatory disease. In another aspect, the invention further provides an antibody or pharmaceutical composition as described herein for use in the described method of treating hMPV infection. In another aspect, the invention further provides an antibody or pharmaceutical composition as described herein for use in the described method of treating hMPV infection.

In another aspect, provided is a method of one or more of detecting, diagnosing, or monitoring respiratory infection), in particular hPMV or PIV1 Infection. For example, the antibodies as described herein can be labeled with a detectable moiety such as an imaging agent and an enzyme-substrate label. The antibodies as described herein can also be used for in vivo diagnostic assays, such as in vivo imaging (e.g., PET or SPECT), or a staining reagent.

With respect to all methods described herein, reference to antibodies also includes pharmaceutical compositions comprising the antibodies and one or more additional agents.

### 8. Administration and Dosing of the Antibodies of the Invention

Typically, an antibody of the invention is administered in an amount effective to treat a condition as described herein. The antibodies the invention can be administered as an antibody per se, or alternatively, as a pharmaceutical composition containing the antibody.

The antibodies of the invention are administered by any suitable route in the form of a pharmaceutical composition adapted to such a route, and in a dose effective for the treatment intended.

In some embodiments, the antibodies may be administered parenterally, for example directly into the bloodstream, into muscle, or into an internal organ. Suitable means for parenteral administration include intravenous, intraarterial, intraperitoneal, intrathecal, intraventricular, intraurethral, intrasternal, intracranial, intramuscular and subcutaneous. In an embodiment, antibodies may be administered subcutaneously. The Suitable devices for parenteral administration include needle (including microneedle) injectors, needle-free injectors, and infusion techniques.

In another embodiment, the compounds of the invention may also be administered topically to the skin or mucosa, that is, dermally or transdermally. In another embodiment, the compounds of the invention can also be administered intranasally or by inhalation. In another embodiment, the compounds of the invention may be administered rectally or vaginally. In another embodiment, the compounds of the invention may also be administered directly to the eye or ear.

The dosage regimen for the antibodies of the invention or compositions containing said antibodies is based on a variety of factors, including the type, age, weight, sex and medical condition of the subject; the severity of the condition; the route of administration; and the activity of the particular antibody employed. Thus, the dosage regimen may vary widely. In one embodiment, the total daily dose of an antibody of the invention is typically from about 0.01 to about 100 mg/kg (e.g., mg antibody of the invention per kg body weight) for the treatment of the indicated conditions discussed herein. In another embodiment, total daily dose of the antibody of the invention is from about 0.1 to about 50 mg/kg, and in another embodiment, from about 0.5 to about 30 mg/kg.

The antibodies of the invention can be used alone, or in combination with one or more other therapeutic agents. The invention provides any of the uses, methods or compositions as defined herein wherein an antibody of the invention is used in combination with one or more other therapeutic agent discussed herein.

The administration of two or more agents “in combination” means that all of the agents are administered closely enough in time to affect treatment of the subject. The two or more agents may be administered simultaneously or sequentially. Additionally, simultaneous administration may be carried out by mixing the agents prior to administration or by administering the agents at the same point in time but as separate dosage forms at the same or different site of administration.

Various formulations of the antibodies of the present invention may be used for administration. In some embodiments, the antibodies may be administered neat. In some embodiments, the antibody and a pharmaceutically acceptable excipient may be in various formulations. Pharmaceutically acceptable excipients are known in the art and are relatively inert substances that facilitate administration of a pharmacologically effective substance. For example, an excipient can give form or consistency, or act as a diluent. Suitable excipients include but are not limited to stabilizing agents, wetting and emulsifying agents, salts for varying osmolarity, encapsulating agents, buffers, and skin penetration enhancers. Excipients as well as formulations for parenteral and nonparenteral drug delivery are set forth in Remington, The Science and Practice of Pharmacy 21st Ed. Mack Publishing, 2005.

In some embodiments, these agents are formulated for administration by injection (e.g., intraperitoneally, intravenously, subcutaneously, intramuscularly, etc.). Accordingly, these agents can be combined with pharmaceutically acceptable vehicles such as saline, Ringer's solution, dextrose solution, and the like. The particular dosage regimen, e.g., dose, timing and repetition, will depend on the particular individual and that individual's medical history.

The antibodies (e.g., anti-hMPV or PIV1 antibodies) as described herein can be administered using any suitable method, including by injection (e.g., intraperitoneally, intravenously, subcutaneously, intramuscularly, etc.). The antibody, e.g., monoclonal antibody or multispecific antibody, also be administered via inhalation, as described herein. Generally, for administration of the antibody of the present, the dosage depends upon the host treated and the particular mode of administration. In one embodiment, the dose range of the antibody of the present invention will be about 0.001 μg/kg body weight to about 20,000 μg/kg body weight. The term “body weight” is applicable when a patient is being treated. When isolated cells are being treated, “body weight” as used herein refers to a “total cell body weight”. The term “total body weight” may be used to apply to both isolated cell and patient treatment. All concentrations and treatment levels are expressed as “body weight” or simply “kg” in this application are also considered to cover the analogous “total cell body weight” and “total body weight” concentrations. However, those of ordinary skill in the art will recognize the utility of a variety of dosage range, for example, 0.01 μg/kg body weight to 20,000 μg/kg body weight, 0.02 μg/kg body weight to 15,000 μg/kg body weight, 0.03 μg/kg body weight to 10,000 μg/kg body weight, 0.04 μg/kg body weight to 5,000 μg/kg body weight, 0.05 μg/kg body weight to 2,500 μg/kg body weight, 0.06 μg/kg body weight to 1,000 μg/kg body weight, 0.07 μg/kg body weight to 500 μg/kg body weight, 0.08 μg/kg body weight to 400 μg/kg body weight, 0.09 μg/kg body weight to 200 μg/kg body weight or 0.1 μg/kg body weight to 100 μg/kg body weight. Further, those of skill will recognize that a variety of different dosage levels will be of use, for example, one or more selected from the group consisting of 0.0001 μg/kg, 0.0002 μg/kg, 0.0003 μg/kg, 0.0004 μg/kg, 0.005 μg/kg, 0.0007 μg/kg, 0.001 μg/kg, 0.1 μg/kg, 1.0 μg/kg, 1.5 μg/kg, 2.0 μg/kg, 5.0 μg/kg, 10.0 μg/kg, 15.0 μg/kg, 30.0 μg/kg, 50 μg/kg, 75 μg/kg, 80 μg/kg, 90 μg/kg, 100 μg/kg, 120 μg/kg, 140 μg/kg, 150 μg/kg, 160 μg/kg, 180 μg/kg, 200 μg/kg, 225 μg/kg, 250 μg/kg, 275 μg/kg, 300 μg/kg, 325 μg/kg, 350 μg/kg, 375 μg/kg, 400 μg/kg, 450 μg/kg, 500 μg/kg, 550 μg/kg, 600 μg/kg, 700 μg/kg, 750 μg/kg, 800 μg/kg, 900 μg/kg, 1 μg/kg, 5 μg/kg, 10 μg/kg, 12 μg/kg, 15 mg/kg, 20 mg/kg, and 30 mg/kg. All of these dosages are exemplary, and any dosage in-between these points is also expected to be of use in the invention. Any of the above dosage ranges or dosage levels may be employed for an antibody of the present invention. For repeated administrations over several days or longer, depending on the condition, the treatment is sustained until a desired suppression of symptoms occurs or until sufficient therapeutic levels are achieved.

Generally, for administration of antibodies provided herein, the candidate dosage can be administered daily, every week, every other week, every three weeks, every four weeks, every five weeks, every six weeks, every seven weeks, every eight weeks, every ten weeks, every twelve weeks, or more than every twelve weeks.

In some embodiments, the candidate dosage is administered daily with the dosage ranging from about any of 1 μg/kg, to 30 μg/kg, to 300 μg/kg, to 3 mg/kg, to 30 mg/kg, to 100 mg/kg or more, depending on the factors mentioned above. For example, daily dosage of about 0.01 mg/kg, about 0.03 mg/kg, about 0.1 mg/kg, about 0.3 mg/kg, about 1 mg/kg, about 2.5 mg/kg, about 3 mg/kg, about 5 mg/kg, about 10 mg/kg, about 15 mg/kg, and about 25 mg/kg may be used.

In some embodiments, the candidate dosage is administered every week with the dosage ranging from about any of 1 μg/kg, to 30 μg/kg, to 300 μg/kg, to 3 mg/kg, to 30 mg/kg, to 100 mg/kg or more, depending on the factors mentioned above. For example, a weekly dosage of about 0.01 mg/kg, about 0.03 mg/kg, about 0.1 mg/kg, about 0.3 mg/kg, about 0.5 mg/kg, about 1 mg/kg, about 2.5 mg/kg, about 3 mg/kg, about 5 mg/kg, about 10 mg/kg, about 15 mg/kg, about 25 mg/kg, and about 30 mg/kg may be used.

In some embodiments, the candidate dosage is administered every two weeks with the dosage ranging from about any of 1 μg/kg, to 30 μg/kg, to 300 μg/kg, to 3 mg/kg, to 30 mg/kg, to 100 mg/kg or more, depending on the factors mentioned above. For example, a bi-weekly dosage of about 0.1 mg/kg, about 0.3 mg/kg, about 1 mg/kg, about 2.5 mg/kg, about 3 mg/kg, about 5 mg/kg, about 10 mg/kg, about 15 mg/kg, about 25 mg/kg, and about 30 mg/kg may be used.

In some embodiments, the candidate dosage is administered every three weeks with the dosage ranging from about any of 1 μg/kg, to 30 μg/kg, to 300 μg/kg, to 3 mg/kg, to 30 mg/kg, to 100 mg/kg or more, depending on the factors mentioned above. For example, a tri-weekly dosage of about 0.1 mg/kg, about 0.3 mg/kg, about 1 mg/kg, about 2.5 mg/kg, about 3 mg/kg, about 5 mg/kg, about 10 mg/kg, about 15 mg/kg, about 25 mg/kg, about 30 mg/kg, about 35 mg/kg, about 40 mg/kg, about 45 mg/kg, and about 50 mg/k may be used.

In some embodiments, the candidate dosage is administered every month or every four weeks with the dosage ranging from about any of 1 μg/kg, to 30 μg/kg, to 300 μg/kg, to 3 mg/kg, to 30 mg/kg, to 100 mg/kg or more, depending on the factors mentioned above. For example, a monthly dosage of about 0.1 mg/kg, about 0.3 mg/kg, about 1 mg/kg, about 2.5 mg/kg, about 3 mg/kg, about 5 mg/kg, about 10 mg/kg, about 15 mg/kg, about 25 mg/kg, about 30 mg/kg, about 35 mg/kg, about 40 mg/kg, about 45 mg/kg, and about 50 mg/kg may be used.

In other embodiments, the candidate dosage is administered daily with the dosage ranging from about 0.01 mg to about 1200 mg or more, depending on the factors mentioned above. For example, daily dosage of about 0.01 mg, about 0.1 mg, about 1 mg, about 10 mg, about 50 mg, about 100 mg, about 200 mg, about 300 mg, about 400 mg, about 500 mg, about 600 mg, about 700 mg, about 800 mg, about 900 mg, about 1000 mg, about 1100 mg, or about 1200 mg may be used. In one embodiment, a daily dosage of between 0.01 mg and 100 mg may be used. In one embodiment, a daily dosage of between 0.01 mg and 1 mg may be used. In one embodiment, a daily dosage of between 0.1 mg and 100 mg may be used. In one embodiment, a daily dosage of between 1 mg and 100 mg may be used.

In other embodiments, the candidate dosage is administered every week with the dosage ranging from about 0.01 mg to about 2000 mg or more, depending on the factors mentioned above. For example, weekly dosage of about 0.01 mg, about 0.1 mg, about 1 mg, about 10 mg, about 50 mg, about 100 mg, about 200 mg, about 300 mg, about 400 mg, about 500 mg, about 600 mg, about 700 mg, about 800 mg, about 900 mg, about 1000 mg, about 1100 mg, about 1200 mg, about 1300 mg, about 1400 mg, about 1500 mg, about 1600 mg, about 1700 mg, about 1800 mg, about 1900 mg, or about 2000 mg may be used.

In one embodiment, a weekly dosage of between 0.01 mg and 0.1 mg may be used. In one embodiment, a weekly dosage of between 0.01 mg and 100 mg may be used. In one embodiment, a weekly dosage of between 0.01 mg and 1 mg may be used. In one embodiment, a weekly dosage of between 0.1 mg and 100 mg may be used. In one embodiment, a weekly dosage of between 1 mg and 100 mg may be used.

In other embodiments, the candidate dosage is administered every two weeks with the dosage ranging from about 0.01 mg to about 2000 mg or more, depending on the factors mentioned above. For example, bi-weekly dosage of about 0.01 mg, about 0.1 mg, about 1 mg, about 10 mg, about 50 mg, about 100 mg, about 200 mg, about 300 mg, about 400 mg, about 500 mg, about 600 mg, about 700 mg, about 800 mg, about 900 mg, about 1000 mg, about 1100 mg, about 1200 mg, about 1300 mg, about 1400 mg, about 1500 mg, about 1600 mg, about 1700 mg, about 1800 mg, about 1900 mg, or about 2000 mg may be used. In one embodiment, a bi-weekly dosage of between 0.01 mg and 0.1 mg may be used. In one embodiment, a bi-weekly dosage of between 0.01 mg and 100 mg may be used. In one embodiment, a bi-weekly dosage of between 0.01 mg and 1 mg may be used. In one embodiment, a bi-weekly dosage of between 0.1 mg and 100 mg may be used. In one embodiment, a bi-weekly dosage of between 1 mg and 100 mg may be used.

In other embodiments, the candidate dosage is administered every three weeks with the dosage ranging from about 0.01 mg to about 2500 mg or more, depending on the factors mentioned above. For example, tri-weekly dosage of about 0.01 mg, about 0.1 mg, about 1 mg, about 10 mg, about 50 mg, about 100 mg, about 200 mg, about 300 mg, about 400 mg, about 500 mg, about 600 mg, about 700 mg, about 800 mg, about 900 mg, about 1000 mg, about 1100 mg, about 1200 mg, about 1300 mg, about 1400 mg, about 1500 mg, about 1600 mg, about 1700 mg, about 1800 mg, about 1900 mg, about 2000 mg, about 2100 mg, about 2200 mg, about 2300 mg, about 2400 mg, or about 2500 mg may be used. In one embodiment, a tri-weekly dosage of between 0.01 mg and 0.1 mg may be used. In one embodiment, a tri-weekly dosage of between 0.01 mg and 100 mg may be used. In one embodiment, a tri-weekly dosage of between 0.01 mg and 1 mg may be used. In one embodiment, a tri-weekly dosage of between 0.1 mg and 100 mg may be used. In one embodiment, a tri-weekly dosage of between 1 mg and 100 mg may be used.

In other embodiments, the candidate dosage is administered every four weeks or month with the dosage ranging from about 0.01 mg to about 3000 mg or more, depending on the factors mentioned above. For example, monthly dosage of about 0.01 mg, about 0.1 mg, about 1 mg, about 10 mg, about 50 mg, about 100 mg, about 200 mg, about 300 mg, about 400 mg, about 500 mg, about 600 mg, about 700 mg, about 800 mg, about 900 mg, about 1000 mg, about 1100 mg, about 1200 mg, about 1300 mg, about 1400 mg, about 1500 mg, about 1600 mg, about 1700 mg, about 1800 mg, about 1900 mg, about 2000 mg, about 2100 mg, about 2200 mg, about 2300 mg, about 2400 mg, about 2500, about 2600 mg, about 2700 mg, about 2800 mg, about 2900 mg, or about 3000 mg may be used. In one embodiment, a monthly dosage of between 0.01 mg and 0.1 mg may be used. In one embodiment, a monthly dosage of between 0.01 mg and 100 mg may be used. In one embodiment, a monthly dosage of between 0.01 mg and 1 mg may be used. In one embodiment, a monthly dosage of between 0.1 mg and 100 mg may be used. In one embodiment, a monthly dosage of between 1 mg and 100 mg may be used.

Other dosage regimens may also be useful, depending on the pattern of pharmacokinetic decay that the practitioner wishes to achieve. In one embodiment, the antibody of the present invention is administered in an initial priming dose followed by a higher and/or continuous, substantially constant dosage. In some embodiments, dosing from one to four times a week is contemplated. In other embodiments, dosing once a month or once every other month or every three months is contemplated. The progress of this therapy is easily monitored by conventional techniques and assays. The dosing regimen can vary over time.

For the purpose of the present invention, the appropriate dosage of an antibody (e.g., one or more of anti-hMPV or PIV1 antibodies) will depend on the antibody or compositions thereof employed, the type and severity of symptoms to be treated, whether the agent is administered for therapeutic purposes, previous therapy, the patient's clinical history and response to the agent, the patient's clearance rate for the administered agent, and the discretion of the attending physician. Typically, the clinician will administer an antibody until a dosage is reached that achieves the desired result. Dose and/or frequency can vary over course of treatment. Empirical considerations, such as the half-life, generally will contribute to the determination of the dosage. For example, antibodies that are compatible with the human immune system, such as humanized antibodies or fully human antibodies, may be used to prolong half-life of the antibody and to prevent the antibody being attacked by the host's immune system. Frequency of administration may be determined and adjusted over the course of therapy, and is generally, but not necessarily, based on treatment and/or suppression and/or amelioration and/or delay of symptoms. Alternatively, sustained continuous release formulations of antibodies may be appropriate. Various formulations and devices for achieving sustained release are known in the art.

In one embodiment, dosages for an antibody (e.g., one or more of anti-hMPV or PIV1 antibodies) may be determined empirically in individuals who have been given one or more administration(s) of the antibody. Individuals are given incremental dosages of an antibody. To assess efficacy, an indicator of the disease can be followed.

In some embodiments, an antibody provided herein (e.g., one or more of anti-hMPV or PIV1 antibodies) may be administered to a subject that has previously received anti-hMPV F protein and/or PIV1 F protein antibodies therapeutic for treatment of a disease. In some embodiments, an antibody provided herein may be an administered to a subject that has previously received anti-hMPV and/or PIV1 antibody therapeutic for treatment of a disease, and for which the previous anti-hMPV and/or PIV1 antibody therapeutic is of limited or no efficacy in the subject (e.g. for which the subject's disease is resistant to treatment with the prior therapeutic).

Administration of an antibody in accordance with the method in the present invention can be continuous or intermittent, depending, for example, upon the recipient's physiological condition, whether the purpose of the administration is therapeutic or prophylactic, and other factors known to skilled practitioners. The administration of an antibody may be ess Therapeutic formulations of the antibody used in accordance with the present invention are prepared for storage by mixing an antibody having the desired degree of purity with optional pharmaceutically acceptable carriers, excipients or stabilizers (Remington, The Science and Practice of Pharmacy 21st Ed. Mack Publishing, 2005), in the form of lyophilized formulations or aqueous solutions. Acceptable carriers, excipients, or stabilizers are nontoxic to recipients at the dosages and concentrations employed, and may comprise buffers such as phosphate, citrate, and other organic acids; salts such as sodium chloride; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens, such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS™ or polyethylene glycol (PEG).entially continuous over a preselected period of time or may be in a series of spaced doses.

### 9. Kits

Another aspect of the invention provides kits comprising the antibody of the invention or pharmaceutical compositions comprising the antibody. A kit may include, in addition to the antibody of the invention or pharmaceutical composition thereof, diagnostic or therapeutic agents. A kit may also include instructions for use in a diagnostic or therapeutic method. In some embodiments, the kit includes the antibody or a pharmaceutical composition thereof and a diagnostic agent. In other embodiments, the kit includes the antibody or a pharmaceutical composition thereof and one or more therapeutic agents.

In yet another embodiment, the invention comprises kits that are suitable for use in performing the methods of treatment described herein. In one embodiment, the kit contains a first dosage form comprising one or more of the antibodies of the invention in quantities sufficient to carry out the methods of the invention. In another embodiment, the kit comprises one or more antibodies of the invention in quantities sufficient to carry out the methods of the invention and at least a first container for a first dosage and a second container for a second dosage.

A further aspect of the invention is a kit comprising anti-hMPV and/or anti-PIV1 antibodies as disclosed herein above and instructions for use in accordance with any of the methods of the invention described herein. Generally, these instructions comprise a description of administration one or more selected from the group consisting of anti-hMPV and/or anti-PIV1 antibodies for the above-described therapeutic treatments.

A further aspect of the invention is a kit comprising anti-hMPV and/or anti-PIV1 protein antibodies as disclosed herein above and instructions for use in accordance with any of the methods of the invention described herein.

TABLE 50
hMPV and PIV1 antibody amino acid sequences

| | SEQ ID NO: of the indicated antibody portion (Amino Acid) | | | | | | | | | |
|---|---|---|---|---|---|---|---|---|---|---|
| | VH | VH | VH | VL | VL | VL | | | | |
| Antibody | CDR1 | CDR2 | CDR3 | CDR1 | CDR2 | CDR3 | VH | VL | HC | LC |
| Anti- | 523 | 525 | 527 | 529 | 530 | 531 | 360 | 361 | 532 | 533 |
| hMPV - | (KABAT) | (KABAT) | (KABAT) | | | | | | | |
| hMPV-2 | 524 | 526 | 528 | | | | | | | |
| mAb | (Chothia) | (Chothia) | (Chothia) | | | | | | | |
| Anti- | 534 | 536 | 538 | 540 | 541 | 542 | 362 | 363 | 543 | 544 |
| PIV1 - | (KABAT) | (KABAT) | (KABAT | | | | | | | |
| PIV1 -8 | 535 | 537 | 539 | | | | | | | |
| mAb | (Chothia) | (Chothia) | (Chothia) | | | | | | | |

TABLE 51
hMPV and PIV1 antibody DNA sequences

| | SEQ ID NO: of the indicated | | | |
|---|---|---|---|---|
| | antibody portion (DNA) | | | |
| Antibody | VH | VL | HC | LC |
| Anti-hMPV - hMPV-2 mAb | 545 | 546 | 547 | 548 |
| Anti-PIV1 - PIV1 - 8 mAb | 549 | 550 | 551 | 552 |

### K. EXAMPLES

The invention is further described by the following illustrative examples. The examples do not limit the invention in any way. They merely serve to clarify the invention.

### Example 1: Design and Preparation of hMPV a F Protein Mutants

This example illustrates the design and preparation of various hMPV A F protein mutants, which include a fibritin foldon trimerization domain and introduced amino acid mutations, such as engineered disulfide bond mutations, cavity-filling mutations, proline substitution mutations, and glycine replacement mutations, or a combination thereof. Exemplary hMPV A F protein mutants, each of which is identified by a unique identifier, such as hMPV043, hMPV044, etc., are provided in Tables 9-16. Each of these mutants was designed and prepared based on the amino acid sequence set forth in SEQ ID NO: 128. Amino acid residues 1-489 of the sequence of SEQ ID NO:128 are identical to amino acid residues 1-489 of the F0 precursor polypeptide of native hMPV A2b as set forth in SEQ ID NO:1. Therefore, the amino acid sequences of these exemplary F protein mutants are identical except for the introduced amino acid mutations as noted for each mutant listed in Tables 9-16. Each of these hMPV F protein mutants comprises two separate polypeptide chains. One of the polypeptide chains, the F2 polypeptide, comprises amino acids 19-102 of SEQ ID NO:128 except for the introduced mutations as noted. The other polypeptide chain comprises the F1 polypeptide (residues 103-489) linked to a foldon trimerization domain (residues 494-520) via a GGGS linker (residues 490-493). The signal peptide (residues 1-18) of SEQ ID NO:128 were cleaved from the F0 precursor during the expression process.

TABLE 9
Exemplary hMPV A F Protein Mutants
Comprising Engineered Disulfide

| | | Amino Acid Sequence |
|---|---|---|
| | | (residues 103-489 |
| | | for F1 polypeptide |
| Mutant | Muta- | and residues 19-102 |
| ID | tions | for F2 polypeptide) |
| hMPV043 | G366C- | F1 (SEQ ID NO: 23): |
| | D454C | FVLGAIALGVATAAAVTAGV |
| | | AIAKTIRLESEVTAIKNALK |
| | | KTNEAVSTLGNGVRVLATAV |
| | | RELKDFVSKNLTRAINKNKC |
| | | DIDDLKMAVSFSQFNRRFLN |
| | | VVRQFSDNAGITPAISLDLM |
| | | TDAELARAVSNMPTSAGQIK |
| | | LMLENRAMVRRKGFGILIGV |
| | | YGSSVIYMVQLPIFGVIDTP |
| | | CWIVKAAPSCSEKKGNYACL |
| | | LREDQGWYCQNAGSTVYYPN |
| | | EKDCETRGDHVFCDTAAGIN |
| | | VAEQSKECNINISTTNYPCK |
| | | VSTCRHPISMVALSPLGALV |
| | | ACYKGVSCSIGSNRVGIIKQ |
| | | LNKGCSYITNQDADTVTIDN |
| | | TVYQLSKVEGEQHVIKGRPV |
| | | SSSFDPVKFPECQFNVALDQ |
| | | VFENIENSQALVDQSNRILS |
| | | SAEKGNT |
| | | F2 (SEQ ID NO: 24): |
| | | LKESYLEESCSTITEGYLSV |
| | | LRTGWYTNVFTLEVGDVENL |
| | | TCADGPSLIKTELDLTKSAL |
| | | RELKTVSADQLAREEQIENP |
| | | RQSR |
| hMPV044 | T411C- | F1 (SEQ ID NO: 25): |
| | Q434C | FVLGAIALGVATAAAVTAGV |
| | | AIAKTIRLESEVTAIKNALK |
| | | KTNEAVSTLGNGVRVLATAV |
| | | RELKDFVSKNLTRAINKNKC |
| | | DIDDLKMAVSFSQFNRRFLN |
| | | VVRQFSDNAGITPAISLDLM |
| | | TDAELARAVSNMPTSAGQIK |
| | | LMLENRAMVRRKGFGILIGV |
| | | YGSSVIYMVQLPIFGVIDTP |
| | | CWIVKAAPSCSEKKGNYACL |
| | | LREDQGWYCQNAGSTVYYPN |
| | | EKDCETRGDHVFCDTAAGIN |
| | | VAEQSKECNINISTTNYPCK |
| | | VSTGRHPISMVALSPLGALV |
| | | ACYKGVSCSIGSNRVGIIKQ |
| | | LNKGCSYICNQDADTVTIDN |
| | | TVYQLSKVEGECHVIKGRPV |
| | | SSSFDPVKFPEDQFNVALDQ |
| | | VFENIENSQALVDQSNRILS |
| | | SAEKGNT |
| | | F2 (SEQ ID NO: 26): |
| | | LKESYLEESCSTITEGYLSV |
| | | LRTGWYTNVFTLEVGDVENL |
| | | TCADGPSLIKTELDLTKSAL |
| | | RELKTVSADQLAREEQIENP |
| | | RQSR |
| hMPV045 | 1137C- | F1 (SEQ ID NO: 27): |
| | A159C | FVLGAIALGVATAAAVTAGV |
| | | AIAKTIRLESEVTACKNALK |
| | | KTNEAVSTLGNGVRVLCTAV |
| | | RELKDFVSKNLTRAINKNKC |
| | | DIDDLKMAVSFSQFNRRFLN |
| | | VVRQFSDNAGITPAISLDLM |
| | | TDAELARAVSNMPTSAGQIK |
| | | LMLENRAMVRRKGFGILIGV |
| | | YGSSVIYMVQLPIFGVIDTP |
| | | CWIVKAAPSCSEKKGNYACL |
| | | LREDQGWYCQNAGSTVYYPN |
| | | EKDCETRGDHVFCDTAAGIN |
| | | VAEQSKECNINISTTNYPCK |
| | | VSTGRHPISMVALSPLGALV |
| | | ACYKGVSCSIGSNRVGIIKQ |
| | | LNKGCSYITNQDADTVTIDN |
| | | TVYQLSKVEGEQHVIKGRPV |
| | | SSSFDPVKFPEDQFNVALDQ |
| | | VFENIENSQALVDQSNRILS |
| | | SAEKGNT |
| | | F2 (SEQ ID NO: 28): |
| | | LKESYLEESCSTITEGYLSV |
| | | LRTGWYTNVFTLEVGDVENL |
| | | TCADGPSLIKTELDLTKSAL |
| | | RELKTVSADQLAREEQIENP |
| | | RQSR |
| hMPV046 | A140C- | F1 (SEQ ID NO: 29): |
| | S149C | FVLGAIALGVATAAAVTAGV |
| | | AIAKTIRLESEVTAIKNCLK |
| | | KTNEAVCTLGNGVRVLATAV |
| | | RELKDFVSKNLTRAINKNKC |
| | | DIDDLKMAVSFSQFNRRFLN |
| | | VVRQFSDNAGITPAISLDLM |
| | | TDAELARAVSNMPTSAGQIK |
| | | LMLENRAMVRRKGFGILIGV |
| | | YGSSVIYMVQLPIFGVIDTP |
| | | CWIVKAAPSCSEKKGNYACL |
| | | LREDQGWYCQNAGSTVYYPN |
| | | EKDCETRGDHVFCDTAAGIN |
| | | VAEQSKECNINISTTNYPCK |
| | | VSTGRHPISMVALSPLGALV |
| | | ACYKGVSCSIGSNRVGIIKQ |
| | | LNKGCSYITNQDADTVTIDN |
| | | TVYQLSKVEGEQHVIKGRPV |
| | | SSSFDPVKFPEDQFNVALDQ |
| | | VFENIENSQALVDQSNRILS |
| | | SAEKGNT |
| | | F2 (SEQ ID NO: 30): |
| | | LKESYLEESCSTITEGYLSV |
| | | LRTGWYTNVFTLEVGDVENL |
| | | TCADGPSLIKTELDLTKSAL |
| | | RELKTVSADQLAREEQIENP |
| | | RQSR |
| hMPV047 | L141C- | F1 (SEQ ID NO: 31): |
| | A159C | FVLGAIALGVATAAAVTAGV |
| | | AIAKTIRLESEVTAIKNACK |
| | | KTNEAVSTLGNGVRVLCTAV |
| | | RELKDFVSKNLTRAINKNKC |
| | | DIDDLKMAVSFSQFNRRFLN |
| | | VVRQFSDNAGITPAISLDLM |
| | | TDAELARAVSNMPTSAGQIK |
| | | LMLENRAMVRRKGFGILIGV |
| | | YGSSVIYMVQLPIFGVIDTP |
| | | CWIVKAAPSCSEKKGNYACL |
| | | LREDQGWYCQNAGSTVYYPN |
| | | EKDCETRGDHVFCDTAAGIN |
| | | VAEQSKECNINISTTNYPCK |
| | | VSTGRHPISMVALSPLGALV |
| | | ACYKGVSCSIGSNRVGIIKQ |
| | | LNKGCSYITNQDADTVTIDN |
| | | TVYQLSKVEGEQHVIKGRPV |
| | | SSSFDPVKFPEDQFNVALDQ |
| | | VFENIENSQALVDQSNRILS |
| | | SAEKGNT |
| | | F2 (SEQ ID NO: 32): |
| | | LKESYLEESCSTITEGYLSV |
| | | LRTGWYTNVFTLEVGDVENL |
| | | TCADGPSLIKTELDLTKSAL |
| | | RELKTVSADQLAREEQIENP |
| | | RQSR |
| hMPV048 | L141C- | F1 (SEQ ID NO: 33): |
| | A161C | FVLGAIALGVATAAAVTAGV |
| | | AIAKTIRLESEVTAIKNACK |
| | | KTNEAVSTLGNGVRVLATCV |
| | | RELKDFVSKNLTRAINKNKC |
| | | DIDDLKMAVSFSQFNRRFLN |
| | | VVRQFSDNAGITPAISLDLM |
| | | TDAELARAVSNMPTSAGQIK |
| | | LMLENRAMVRRKGFGILIGV |
| | | YGSSVIYMVQLPIFGVIDTP |
| | | CWIVKAAPSCSEKKGNYACL |
| | | LREDQGWYCQNAGSTVYYPN |
| | | EKDCETRGDHVFCDTAAGIN |
| | | VAEQSKECNINISTTNYPCK |
| | | VSTGRHPISMVALSPLGALV |
| | | ACYKGVSCSIGSNRVGIIKQ |
| | | LNKGCSYITNQDADTVTIDN |
| | | TVYQLSKVEGEQHVIKGRPV |
| | | SSSFDPVKFPEDQFNVALDQ |
| | | VFENIENSQALVDQSNRILS |
| | | SAEKGNT |
| | | F2 (SEQ ID NO: 34): |
| | | LKESYLEESCSTITEGYLSV |
| | | LRTGWYTNVFTLEVGDVENL |
| | | TCADGPSLIKTELDLTKSAL |
| | | RELKTVSADQLAREEQIENP |
| | | RQSR |
| hMPV049 | E146C- | F1 (SEQ ID NO: 35): |
| | T160C | FVLGAIALGVATAAAVTAGV |
| | | AIAKTIRLESEVTAIKNALK |
| | | KTNCAVSTLGNGVRVLACAV |
| | | RELKDFVSKNLTRAINKNKC |
| | | DIDDLKMAVSFSQFNRRFLN |
| | | VVRQFSDNAGITPAISLDLM |
| | | TDAELARAVSNMPTSAGQIK |
| | | LMLENRAMVRRKGFGILIGV |
| | | YGSSVIYMVQLPIFGVIDTP |
| | | CWIVKAAPSCSEKKGNYACL |
| | | LREDQGWYCQNAGSTVYYPN |
| | | EKDCETRGDHVFCDTAAGIN |
| | | VAEQSKECNINISTTNYPCK |
| | | VSTGRHPISMVALSPLGALV |
| | | ACYKGVSCSIGSNRVGIIKQ |
| | | LNKGCSYITNQDADTVTIDN |
| | | TVYQLSKVEGEQHVIKGRPV |
| | | SSSFDPVKFPEDQFNVALDQ |
| | | VFENIENSQALVDQSNRILS |
| | | SAEKGNT |
| | | F2 (SEQ ID NO: 36): |
| | | LKESYLEESCSTITEGYLSV |
| | | LRTGWYTNVFTLEVGDVENL |
| | | TCADGPSLIKTELDLTKSAL |
| | | RELKTVSADQLAREEQIENP |
| | | RQSR |
| hMPV050 | V148C- | F1 (SEQ ID NO: 37): |
| | L158C | FVLGAIALGVATAAAVTAGV |
| | | AIAKTIRLESEVTAIKNALK |
| | | KTNEACSTLGNGVRVCATAV |
| | | RELKDFVSKNLTRAINKNKC |
| | | DIDDLKMAVSFSQFNRRFLN |
| | | VVRQFSDNAGITPAISLDLM |
| | | TDAELARAVSNMPTSAGQIK |
| | | LMLENRAMVRRKGFGILIGV |
| | | YGSSVIYMVQLPIFGVIDTP |
| | | CWIVKAAPSCSEKKGNYACL |
| | | LREDQGWYCQNAGSTVYYPN |
| | | EKDCETRGDHVFCDTAAGIN |
| | | VAEQSKECNINISTTNYPCK |
| | | VSTGRHPISMVALSPLGALV |
| | | ACYKGVSCSIGSNRVGIIKQ |
| | | LNKGCSYITNQDADTVTIDN |
| | | TVYQLSKVEGEQHVIKGRPV |
| | | SSSFDPVKFPEDQFNVALDQ |
| | | VFENIENSQALVDQSNRILS |
| | | SAEKGNT |
| | | F2 (SEQ ID NO: 38): |
| | | LKESYLEESCSTITEGYLSV |
| | | LRTGWYTNVFTLEVGDVENL |
| | | TCADGPSLIKTELDLTKSAL |
| | | RELKTVSADQLAREEQIENP |
| | | RQSR |
| hMPV077 | Q476C, | F1 (SEQ ID NO: 386) |
| | S477C | FVLGAIALGVATAAAVTAGV |
| | | AIAKTIRLESEVTAIKNALK |
| | | KTNEAVSTLGNGVRVLATAV |
| | | RELKDFVSKNLTRAINKNKC |
| | | DIDDLKMAVSFSQFNRRFLN |
| | | VVRQFSDNAGITPAISLDLM |
| | | TDAELARAVSNMPTSAGQIK |
| | | LMLENRAMVRRKGFGILIGV |
| | | YGSSVIYMVQLPIFGVIDTP |
| | | CWIVKAAPSCSEKKGNYACL |
| | | LREDQGWYCQNAGSTVYYPN |
| | | EKDCETRGDHVFCDTAAGIN |
| | | VAEQSKECNINISTTNYPCK |
| | | VSTGRHPISMVALSPLGALV |
| | | ACYKGVSCSIGSNRVGIIKQ |
| | | LNKGCSYITNQDADTVTIDN |
| | | TVYQLSKVEGEQHVIKGRPV |
| | | SSSFDPVKFPEDQFNVALDQ |
| | | VFENIENSQALVDCCNRILS |
| | | SAEKGNT |
| | | F2 (SEQ ID NO: 387) |
| | | LKESYLEESCSTITEGYLSV |
| | | LRTGWYTNVFTLEVGDVENL |
| | | TCADGPSLIKTELDLTKSAL |
| | | RELKTVSADQLAREEQIENP |
| | | RQSR |
| hMPV078 | T411C- | F1 (SEQ ID NO: 39): |
| | Q434C | FVLGAIALGVATAAAVTAGV |
| | A140C- | AIAKTIRLESEVTAIKNCLK |
| | S149C | KTNEAVCTLGNGVRVLATAV |
| | | RELKDFVSKNLTRAINKNKC |
| | | DIDDLKMAVSFSQFNRRFLN |
| | | VVRQFSDNAGITPAISLDLM |
| | | TDAELARAVSNMPTSAGQIK |
| | | LMLENRAMVRRKGFGILIGV |
| | | YGSSVIYMVQLPIFGVIDTP |
| | | CWIVKAAPSCSEKKGNYACL |
| | | LREDQGWYCQNAGSTVYYPN |
| | | EKDCETRGDHVFCDTAAGIN |
| | | VAEQSKECNINISTTNYPCK |
| | | VSTGRHPISMVALSPLGALV |
| | | ACYKGVSCSIGSNRVGIIKQ |
| | | LNKGCSYICNQDADTVTIDN |
| | | TVYQLSKVEGECHVIKGRPV |
| | | SSSFDPVKFPEDQFNVALDQ |
| | | VFENIENSQALVDQSNRILS |
| | | SAEKGNT |
| | | F2 (SEQ ID NO: 40): |
| | | LKESYLEESCSTITEGYLSV |
| | | LRTGWYTNVFTLEVGDVENL |
| | | TCADGPSLIKTELDLTKSAL |
| | | RELKTVSADQLAREEQIENP |
| | | RQSR |
| hMPV084 | T411C- | F1 (SEQ ID NO: 41): |
| | Q434C | FVLGAIALGVATAAAVTAGV |
| | L141C- | AIAKTIRLESEVTAIKNACK |
| | A161C | KTNEAVSTLGNGVRVLATCV |
| | | RELKDFVSKNLTRAINKNKC |
| | | DIDDLKMAVSFSQFNRRFLN |
| | | VVRQFSDNAGITPAISLDLM |
| | | TDAELARAVSNMPTSAGQIK |
| | | LMLENRAMVRRKGFGILIGV |
| | | YGSSVIYMVQLPIFGVIDTP |
| | | CWIVKAAPSCSEKKGNYACL |
| | | LREDQGWYCQNAGSTVYYPN |
| | | EKDCETRGDHVFCDTAAGIN |
| | | VAEQSKECNINISTTNYPCK |
| | | VSTGRHPISMVALSPLGALV |
| | | ACYKGVSCSIGSNRVGIIKQ |
| | | LNKGCSYICNQDADTVTIDN |
| | | TVYQLSKVEGECHVIKGRPV |
| | | SSSFDPVKFPEDQFNVALDQ |
| | | VFENIENSQALVDQSNRILS |
| | | SAEKGNT |
| | | F2 (SEQ ID NO: 42): |
| | | LKESYLEESCSTITEGYLSV |
| | | LRTGWYTNVFTLEVGDVENL |
| | | TCADGPSLIKTELDLTKSAL |
| | | RELKTVSADQLAREEQIENP |
| | | RQS |
| | | R |

TABLE 10
Exemplary hMPV A F Protein Mutants
Comprising proline substitution mutations

| | | Amino Acid Sequence |
|---|---|---|
| | | (residues 103-489 for |
| | | F1 polypeptide |
| Mutant | Muta- | and residues 19-102 |
| ID | tions | for F2 polypeptide) |
| hMPV052 | L66P | F1 (SEQ ID NO: 43): |
| | | FVLGAIALGVATAAAVTAGVAIAKTIRLES |
| | | EVTAIKNALKKTNEAVSTLGNGVRVLATAV |
| | | RELKDFVSKNLTRAINKNKCDIDDLKMAVS |
| | | FSQFNRRFLNVVRQFSDNAGITPAISLDLM |
| | | TDAELARAVSNMPTSAGQIKLMLENRAMVR |
| | | RKGFGILIGVYGSSVIYMVQLPIFGVIDTP |
| | | CWIVKAAPSCSEKKGNYACLLREDQGWYCQ |
| | | NAGSTVYYPNEKDCETRGDHVFCDTAAGIN |
| | | VAEQSKECNINISTTNYPCKVSTGRHPISM |
| | | VALSPLGALVACYKGVSCSIGSNRVGIIKQ |
| | | LNKGCSYITNQDADTVTIDNTVYQLSKVEG |
| | | EQHVIKGRPVSSSFDPVKFPEDQFNVALDQ |
| | | VFENIENSQALVDQSNRILSSAEKGNT |
| | | F2 (SEQ ID NO: 44): |
| | | LKESYLEESCSTITEGYLSVLRTGWYTNVF |
| | | TLEVGDVENLTCADGPSPIKTELDLTKSAL |
| | | RELKTVSADQLAREEQIENPRQSR |
| hMPV053 | L110P | F1 (SEQ ID NO: 45): |
| | | FVLGAIAPGVATAAAVTAGVAIAKTIRLES |
| | | EVTAIKNALKKTNEAVSTLGNGVRVLATAV |
| | | RELKDFVSKNLTRAINKNKCDIDDLKMAVS |
| | | FSQFNRRFLNVVRQFSDNAGITPAISLDLM |
| | | TDAELARAVSNMPTSAGQIKLMLENRAMVR |
| | | RKGFGILIGVYGSSVIYMVQLPIFGVIDTP |
| | | CWIVKAAPSCSEKKGNYACLLREDQGWYCQ |
| | | NAGSTVYYPNEKDCETRGDHVFCDTAAGIN |
| | | VAEQSKECNINISTTNYPCKVSTGRHPISM |
| | | VALSPLGALVACYKGVSCSIGSNRVGIIKQ |
| | | LNKGCSYITNQDADTVTIDNTVYQLSKVEG |
| | | EQHVIKGRPVSSSFDPVKFPEDQFNVALDQ |
| | | VFENIENSQALVDQSNRILSSAEKGNT |
| | | F2 (SEQ ID NO: 46): |
| | | LKESYLEESCSTITEGYLSVLRTGWYTNVF |
| | | TLEVGDVENLTCADGPSLIKTELDLTKSAL |
| | | RELKTVSADQLAREEQIENPRQSR |
| hMPV054 | S132P | F1 (SEQ ID NO: 47): |
| | | FVLGAIALGVATAAAVTAGVAIAKTIRLEP |
| | | EVTAIKNALKKTNEAVSTLGNGVRVLATAV |
| | | RELKDFVSKNLTRAINKNKCDIDDLKMAVS |
| | | FSQFNRRFLNVVRQFSDNAGITPAISLDLM |
| | | TDAELARAVSNMPTSAGQIKLMLENRAMVR |
| | | RKGFGILIGVYGSSVIYMVQLPIFGVIDTP |
| | | CWIVKAAPSCSEKKGNYACLLREDQGWYCQ |
| | | NAGSTVYYPNEKDCETRGDHVFCDTAAGIN |
| | | VAEQSKECNINISTTNYPCKVSTGRHPISM |
| | | VALSPLGALVACYKGVSCSIGSNRVGIIKQ |
| | | LNKGCSYITNQDADTVTIDNTVYQLSKVEG |
| | | EQHVIKGRPVSSSFDPVKFPEDQFNVALDQ |
| | | VFENIENSQALVDQSNRILSSAEKGNT |
| | | F2 (SEQ ID NO: 48): |
| | | LKESYLEESCSTITEGYLSVLRTGWYTNVF |
| | | TLEVGDVENLTCADGPSLIKTELDLTKSAL |
| | | RELKTVSADQLAREEQIENPRQSR |
| hMPV055 | N145P | F1 (SEQ ID NO: 49): |
| | | FVLGAIALGVATAAAVTAGVAIAKTIRLES |
| | | EVTAIKNALKKTPEAVSTLGNGVRVLATAV |
| | | RELKDFVSKNLTRAINKNKCDIDDLKMAVS |
| | | FSQFNRRFLNVVRQFSDNAGITPAISLDLM |
| | | TDAELARAVSNMPTSAGQIKLMLENRAMVR |
| | | RKGFGILIGVYGSSVIYMVQLPIFGVIDTP |
| | | CWIVKAAPSCSEKKGNYACLLREDQGWYCQ |
| | | NAGSTVYYPNEKDCETRGDHVFCDTAAGIN |
| | | VAEQSKECNINISTTNYPCKVSTGRHPISM |
| | | VALSPLGALVACYKGVSCSIGSNRVGIIKQ |
| | | LNKGCSYITNQDADTVTIDNTVYQLSKVEG |
| | | EQHVIKGRPVSSSFDPVKFPEDQFNVALDQ |
| | | VFENIENSQALVDQSNRILSSAEKGNT |
| | | F2 (SEQ ID NO: 50): |
| | | LKESYLEESCSTITEGYLSVLRTGWYTNVF |
| | | TLEVGDVENLTCADGPSLIKTELDLTKSAL |
| | | RELKTVSADQLAREEQIENPRQSR |
| hMPV058 | L187P | F1 (SEQ ID NO: 51): |
| | | FVLGAIALGVATAAAVTAGVAIAKTIRLES |
| | | EVTAIKNALKKTNEAVSTLGNGVRVLATAV |
| | | RELKDFVSKNLTRAINKNKCDIDDPKMAVS |
| | | FSQFNRRFLNVVRQFSDNAGITPAISLDLM |
| | | TDAELARAVSNMPTSAGQIKLMLENRAMVR |
| | | RKGFGILIGVYGSSVIYMVQLPIFGVIDTP |
| | | CWIVKAAPSCSEKKGNYACLLREDQGWYCQ |
| | | NAGSTVYYPNEKDCETRGDHVFCDTAAGIN |
| | | VAEQSKECNINISTTNYPCKVSTGRHPISM |
| | | VALSPLGALVACYKGVSCSIGSNRVGIIKQ |
| | | LNKGCSYITNQDADTVTIDNTVYQLSKVEG |
| | | EQHVIKGRPVSSSFDPVKFPEDQFNVALDQ |
| | | VFENIENSQALVDQSNRILSSAEKGNT |
| | | F2 (SEQ ID NO: 52): |
| | | LKESYLEESCSTITEGYLSVLRTGWYTNVF |
| | | TLEVGDVENLTCADGPSLIKTELDLTKSAL |
| | | RELKTVSADQLAREEQIENPRQSR |
| hMPV059 | V449P | F1 (SEQ ID NO: 53): |
| | | FVLGAIALGVATAAAVTAGVAIAKTIRLES |
| | | EVTAIKNALKKTNEAVSTLGNGVRVLATAV |
| | | RELKDFVSKNLTRAINKNKCDIDDLKMAVS |
| | | FSQFNRRFLNVVRQFSDNAGITPAISLDLM |
| | | TDAELARAVSNMPTSAGQIKLMLENRAMVR |
| | | RKGFGILIGVYGSSVIYMVQLPIFGVIDTP |
| | | CWIVKAAPSCSEKKGNYACLLREDQGWYCQ |
| | | NAGSTVYYPNEKDCETRGDHVFCDTAAGIN |
| | | VAEQSKECNINISTTNYPCKVSTGRHPISM |
| | | VALSPLGALVACYKGVSCSIGSNRVGIIKQ |
| | | LNKGCSYITNQDADTVTIDNTVYQLSKVEG |
| | | EQHVIKGRPVSSSFDPPKFPEDQFNVALDQ |
| | | VFENIENSQALVDQSNRILSSAEKGNT |
| | | F2 (SEQ ID NO: 54): |
| | | LKESYLEESCSTITEGYLSVLRTGWYTNVF |
| | | TLEVGDVENLTCADGPSLIKTELDLTKSAL |
| | | RELKTVSADQLAREEQIENPRQSR |
| hMPV060 | A459P | F1 (SEQ ID NO: 55): |
| | | FVLGAIALGVATAAAVTAGVAIAKTIRLES |
| | | EVTAIKNALKKTNEAVSTLGNGVRVLATAV |
| | | RELKDFVSKNLTRAINKNKCDIDDLKMAVS |
| | | FSQFNRRFLNVVRQFSDNAGITPAISLDLM |
| | | TDAELARAVSNMPTSAGQIKLMLENRAMVR |
| | | RKGFGILIGVYGSSVIYMVQLPIFGVIDTP |
| | | CWIVKAAPSCSEKKGNYACLLREDQGWYCQ |
| | | NAGSTVYYPNEKDCETRGDHVFCDTAAGIN |
| | | VAEQSKECNINISTTNYPCKVSTGRHPISM |
| | | VALSPLGALVACYKGVSCSIGSNRVGIIKQ |
| | | LNKGCSYITNQDADTVTIDNTVYQLSKVEG |
| | | EQHVIKGRPVSSSFDPVKFPEDQFNVPLDQ |
| | | VFENIENSQALVDQSNRILSSAEKGNT |
| | | F2 (SEQ ID NO: 56): |
| | | LKESYLEESCSTITEGYLSVLRTGWYTNVF |
| | | TLEVGDVENLTCADGPSLIKTELDLTKSAL |
| | | RELKTVSADQLAREEQIENPRQS |
| | | R |

TABLE 11
Exemplary hMPV A F Protein Mutants Comprising
glycine replacement mutation

| | | Amino Acid Sequence |
|---|---|---|
| | | (residues 103-489 for |
| | | F1 polypeptide |
| Mutant | Muta- | and residues 19-102 |
| ID | tions | for F2 polypeptide) |
| hMPV061 | G106A | F1 (SEQ ID NO: 57): |
| | | FVLAAIALGVATAAAVTAGVAIAKTIRLES |
| | | EVTAIKNALKKTNEAVSTLGNGVRVLATAV |
| | | RELKDFVSKNLTRAINKNKCDIDDLKMAVS |
| | | FSQFNRRFLNVVRQFSDNAGITPAISLDLM |
| | | TDAELARAVSNMPTSAGQIKLMLENRAMVR |
| | | RKGFGILIGVYGSSVIYMVQLPIFGVIDTP |
| | | CWIVKAAPSCSEKKGNYACLLREDQGWYCQ |
| | | NAGSTVYYPNEKDCETRGDHVFCDTAAGIN |
| | | VAEQSKECNINISTTNYPCKVSTGRHPISM |
| | | VALSPLGALVACYKGVSCSIGSNRVGIIKQ |
| | | LNKGCSYITNQDADTVTIDNTVYQLSKVEG |
| | | EQHVIKGRPVSSSFDPVKFPEDQFNVALDQ |
| | | VFENIENSQALVDQSNRILSSAEKGNT |
| | | F2 (SEQ ID NO: 58): |
| | | LKESYLEESCSTITEGYLSVLRTGWYTNVF |
| | | TLEVGDVENLTCADGPSLIKTELDLTKSAL |
| | | RELKTVSADQLAREEQIENPRQSR |
| hMPV062 | G121A | F1 (SEQ ID NO: 59): |
| | | FVLGAIALGVATAAAVTAAVAIAKTIRLES |
| | | EVTAIKNALKKTNEAVSTLGNGVRVLATAV |
| | | RELKDFVSKNLTRAINKNKCDIDDLKMAVS |
| | | FSQFNRRFLNVVRQFSDNAGITPAISLDLM |
| | | TDAELARAVSNMPTSAGQIKLMLENRAMVR |
| | | RKGFGILIGVYGSSVIYMVQLPIFGVIDTP |
| | | CWIVKAAPSCSEKKGNYACLLREDQGWYCQ |
| | | NAGSTVYYPNEKDCETRGDHVFCDTAAGIN |
| | | VAEQSKECNINISTTNYPCKVSTGRHPISM |
| | | VALSPLGALVACYKGVSCSIGSNRVGIIKQ |
| | | LNKGCSYITNQDADTVTIDNTVYQLSKVEG |
| | | EQHVIKGRPVSSSFDPVKFPEDQFNVALDQ |
| | | VFENIENSQALVDQSNRILSSAEKGNT |
| | | F2 (SEQ ID NO: 60): |
| | | LKESYLEESCSTITEGYLSVLRTGWYTNVF |
| | | TLEVGDVENLTCADGPSLIKTELDLTKSAL |
| | | RELKTVSADQLAREEQIENPRQSR |
| hMPV063 | G239A | F1 (SEQ ID NO: 61): |
| | | FVLGAIALGVATAAAVTAGVAIAKTIRLES |
| | | EVTAIKNALKKTNEAVSTLGNGVRVLATAV |
| | | RELKDFVSKNLTRAINKNKCDIDDLKMAVS |
| | | FSQFNRRFLNVVRQFSDNAGITPAISLDLM |
| | | TDAELARAVSNMPTSAAQIKLMLENRAMVR |
| | | RKGFGILIGVYGSSVIYMVQLPIFGVIDTP |
| | | CWIVKAAPSCSEKKGNYACLLREDQGWYCQ |
| | | NAGSTVYYPNEKDCETRGDHVFCDTAAGIN |
| | | VAEQSKECNINISTTNYPCKVSTGRHPISM |
| | | VALSPLGALVACYKGVSCSIGSNRVGIIKQ |
| | | LNKGCSYITNQDADTVTIDNTVYQLSKVEG |
| | | EQHVIKGRPVSSSFDPVKFPEDQFNVALDQ |
| | | VFENIENSQALVDQSNRILSSAEKGN |
| | | F2 (SEQ ID NO: 62): |
| | | LKESYLEESCSTITEGYLSVLRTGWYTNVF |
| | | TLEVGDVENLTCADGPSLIKTELDLTKSAL |
| | | RELKTVSADQLAREEQIENPRQS |
| | | R |

TABLE 12
Exemplary hMPV A F Protein Mutants Comprising
Cavity Filling Mutations

| | | Amino Acid Sequence |
|---|---|---|
| | | (residues 103-489 for |
| | | F1 polypeptide |
| Mutant | Muta- | and residues 19-102 |
| ID | tions | for F2 polypeptide) |
| hMPV066 | T491 | F1 (SEQ ID NO: 63): |
| | | FVLGAIALGVATAAAVTAGVAIAKTIRLES |
| | | EVTAIKNALKKTNEAVSTLGNGVRVLATAV |
| | | RELKDFVSKNLTRAINKNKCDIDDLKMAVS |
| | | FSQFNRRFLNVVRQFSDNAGITPAISLDLM |
| | | TDAELARAVSNMPTSAGQIKLMLENRAMVR |
| | | RKGFGILIGVYGSSVIYMVQLPIFGVIDTP |
| | | CWIVKAAPSCSEKKGNYACLLREDQGWYCQ |
| | | NAGSTVYYPNEKDCETRGDHVFCDTAAGIN |
| | | VAEQSKECNINISTTNYPCKVSTGRHPISM |
| | | VALSPLGALVACYKGVSCSIGSNRVGIIKQ |
| | | LNKGCSYITNQDADTVTIDNTVYQLSKVEG |
| | | EQHVIKGRPVSSSFDPVKFPEDQFNVALDQ |
| | | VFENIENSQALVDQSNRILSSAEKGNT |
| | | F2 (SEQ ID NO: 64): |
| | | LKESYLEESCSTITEGYLSVLRTGWYTNVF |
| | | ILEVGDVENLTCADGPSLIKTELDLTKSAL |
| | | RELKTVSADQLAREEQIENPRQSR |
| hMPV067 | S149T | F1 (SEQ ID NO: 65): |
| | | FVLGAIALGVATAAAVTAGVAIAKTIRLES |
| | | EVTAIKNALKKTNEAVTTLGNGVRVLATAV |
| | | RELKDFVSKNLTRAINKNKCDIDDLKMAVS |
| | | FSQFNRRFLNVVRQFSDNAGITPAISLDLM |
| | | TDAELARAVSNMPTSAGQIKLMLENRAMVR |
| | | RKGFGILIGVYGSSVIYMVQLPIFGVIDTP |
| | | CWIVKAAPSCSEKKGNYACLLREDQGWYCQ |
| | | NAGSTVYYPNEKDCETRGDHVFCDTAAGIN |
| | | VAEQSKECNINISTTNYPCKVSTGRHPISM |
| | | VALSPLGALVACYKGVSCSIGSNRVGIIKQ |
| | | LNKGCSYITNQDADTVTIDNTVYQLSKVEG |
| | | EQHVIKGRPVSSSFDPVKFPEDQFNVALDQ |
| | | VFENIENSQALVDQSNRILSSAEKGNT |
| | | F2 (SEQ ID NO: 66): |
| | | LKESYLEESCSTITEGYLSVLRTGWYTNVF |
| | | TLEVGDVENLTCADGPSLIKTELDLTKSAL |
| | | RELKTVSADQLAREEQIENPRQSR |
| hMPV068 | A159V | F1 (SEQ ID NO: 67): |
| | | FVLGAIALGVATAAAVTAGVAIAKTIRLES |
| | | EVTAIKNALKKTNEAVSTLGNGVRVLVTAV |
| | | RELKDFVSKNLTRAINKNKCDIDDLKMAVS |
| | | FSQFNRRFLNVVRQFSDNAGITPAISLDLM |
| | | TDAELARAVSNMPTSAGQIKLMLENRAMVR |
| | | RKGFGILIGVYGSSVIYMVQLPIFGVIDTP |
| | | CWIVKAAPSCSEKKGNYACLLREDQGWYCQ |
| | | NAGSTVYYPNEKDCETRGDHVFCDTAAGIN |
| | | VAEQSKECNINISTTNYPCKVSTGRHPISM |
| | | VALSPLGALVACYKGVSCSIGSNRVGIIKQ |
| | | LNKGCSYITNQDADTVTIDNTVYQLSKVEG |
| | | EQHVIKGRPVSSSFDPVKFPEDQFNVALDQ |
| | | VFENIENSQALVDQSNRILSSAEKGNT |
| | | F2 (SEQ ID NO: 68): |
| | | LKESYLEESCSTITEGYLSVLRTGWYTNVF |
| | | TLEVGDVENLTCADGPSLIKTELDLTKSAL |
| | | RELKTVSADQLAREEQIENPRQSR |
| hMPV069 | S2911 | F1 (SEQ ID NO: 69): |
| | | FVLGAIALGVATAAAVTAGVAIAKTIRLES |
| | | EVTAIKNALKKTNEAVSTLGNGVRVLATAV |
| | | RELKDFVSKNLTRAINKNKCDIDDL |
| | | KMAVSFSQFNRRFLNVVRQFSDNAGITPAI |
| | | SLDLMTDAELARAVSNMPTSAGQIKLMLEN |
| | | RAMVRRKGFGILIGVYGSSVIYMVQLPIFG |
| | | VIDTPCWIVKAAPICSEKKGNYACLLREDQ |
| | | GWYCQNAGSTVYYPNEKDCETRGDHVFCDT |
| | | AAGINVAEQSKECNINISTTNYPCKVSTGR |
| | | HPISMVALSPLGALVACYKGVSCSIGSNRV |
| | | GIIKQLNKGCSYITNQDADTVTIDNTVYQL |
| | | SKVEGEQHVIKGRPVSSSFDPVKFPEDQFN |
| | | VALDQVFENIENSQALVDQSNRILSSAEKG |
| | | NT |
| | | F2 (SEQ ID NO: 70): |
| | | LKESYLEESCSTITEGYLSVLRTGWYTNVF |
| | | TLEVGDVENLTCADGPSLIKTELDLTKSAL |
| | | RELKTVSADQLAREEQIENPRQSR |
| hMPV071 | T3651 | F1 (SEQ ID NO: 71): |
| | | FVLGAIALGVATAAAVTAGVAIAKTIRLES |
| | | EVTAIKNALKKTNEAVSTLGNGVRVLATAV |
| | | RELKDFVSKNLTRAINKNKCDIDDLKMAVS |
| | | FSQFNRRFLNVVRQFSDNAGITPAISLDLM |
| | | TDAELARAVSNMPTSAGQIKLMLENRAMVR |
| | | RKGFGILIGVYGSSVIYMVQLPIFGVIDTP |
| | | CWIVKAAPSCSEKKGNYACLLREDQGWYCQ |
| | | NAGSTVYYPNEKDCETRGDHVFCDTAAGIN |
| | | VAEQSKECNINISTTNYPCKVSIGRHPISM |
| | | VALSPLGALVACYKGVSCSIGSNRVGIIKQ |
| | | LNKGCSYITNQDADTVTIDNTVYQLSKVEG |
| | | EQHVIKGRPVSSSFDPVKFPEDQFNVALDQ |
| | | VFENIENSQALVDQSNRILSSAEKGNT |
| | | F2 (SEQ ID NO: 72): |
| | | LKESYLEESCSTITEGYLSVLRTGWYTNVF |
| | | TLEVGDVENLTCADGPSLIKTELDLTKSAL |
| | | RELKTVSADQLAREEQIENPRQSR |
| hMPV075 | L473F | F1 (SEQ ID NO: 73): |
| | | FVLGAIALGVATAAAVTAGVAIAKTIRLES |
| | | EVTAIKNALKKTNEAVSTLGNGVRVLATAV |
| | | RELKDFVSKNLTRAINKNKCDIDDLKMAVS |
| | | FSQFNRRFLNVVRQFSDNAGITPAISLDLM |
| | | TDAELARAVSNMPTSAGQIKLMLENRAMVR |
| | | RKGFGILIGVYGSSVIYMVQLPIFGVIDTP |
| | | CWIVKAAPSCSEKKGNYACLLREDQGWYCQ |
| | | NAGSTVYYPNEKDCETRGDHVFCDTAAGIN |
| | | VAEQSKECNINISTTNYPCKVSTGRHPISM |
| | | VALSPLGALVACYKGVSCSIGSNRVGIIKQ |
| | | LNKGCSYITNQDADTVTIDNTVYQLSKVEG |
| | | EQHVIKGRPVSSSFDPVKFPEDQFNVALDQ |
| | | VFENIENSQAFVDQSNRILSSAEKGNT |
| | | F2 (SEQ ID NO: 74): |
| | | LKESYLEESCSTITEGYLSVLRTGWYTNVF |
| | | TLEVGDVENLTCADGPSLIKTELDLTKSAL |
| | | RELKTVSADQLAREEQIENPRQS |
| | | R |

TABLE 13
Exemplary hMPV A F Protein Mutants
Comprising Engineered Disulfide Mutations
and proline substitution mutations

| | | Amino Acid Sequence |
|---|---|---|
| | | (residues 103-489 |
| | | for F1 polypeptide |
| Mutant | Muta- | and residues 19-102 |
| ID | tions | for F2 polypeptide) |
| hMPV079 | T411C- | F1 (SEQ ID NO: 75): |
| | Q434C, | FVLGAIALGVATAAAVTAGVAIAKTIRLES |
| | A140C- | EVTAIKNCLKKTNEAVCTLGNGVRVLATAV |
| | S149C, | RELKDFVSKNLTRAINKNKCDIDDLKMAVS |
| | A459P | FSQFNRRFLNVVRQFSDNAGITPAISLDLM |
| | | TDAELARAVSNMPTSAGQIKLMLENRAMVR |
| | | RKGFGILIGVYGSSVIYMVQLPIFGVIDTP |
| | | CWIVKAAPSCSEKKGNYACLLREDQGWYCQ |
| | | NAGSTVYYPNEKDCETRGDHVFCDTAAGIN |
| | | VAEQSKECNINISTTNYPCKVSTGRHPISM |
| | | VALSPLGALVACYKGVSCSIGSNRVGIIKQ |
| | | LNKGCSYICNQDADTVTIDNTVYQLSKVEG |
| | | ECHVIKGRPVSSSFDPVKFPEDQFNVPLDQ |
| | | VFENIENSQALVDQSNRILSSAEKGNT |
| | | F2 (SEQ ID NO: 76): |
| | | LKESYLEESCSTITEGYLSVLRTGWYTNVF |
| | | TLEVGDVENLTCADGPSLIKTELDLTKSAL |
| | | RELKTVSADQLAREEQIENPRQSR |
| hMPV085 | T411C- | F1 (SEQ ID NO: 77): |
| | Q434C, | FVLGAIALGVATAAAVTAGVAIAKTIRLES |
| | L141C- | EVTAIKNACKKTNEAVSTLGNGVRVLATCV |
| | A161C, | RELKDFVSKNLTRAINKNKCDIDDLKMAVS |
| | A459P | FSQFNRRFLNVVRQFSDNAGITPAISLDLM |
| | | TDAELARAVSNMPTSAGQIKLMLENRAMVR |
| | | RKGFGILIGVYGSSVIYMVQLPIFGVIDTP |
| | | CWIVKAAPSCSEKKGNYACLLREDQGWYCQ |
| | | NAGSTVYYPNEKDCETRGDHVFCDTAAGIN |
| | | VAEQSKECNINISTTNYPCKVSTGRHPISM |
| | | VALSPLGALVACYKGVSCSIGSNRVGIIKQ |
| | | LNKGCSYICNQDADTVTIDNTVYQLSKVEG |
| | | ECHVIKGRPVSSSFDPVKFPEDQFNVPLDQ |
| | | VFENIENSQALVDQSNRILSSAEKGNT |
| | | F2 (SEQ ID NO: 78): |
| | | LKESYLEESCSTITEGYLSVLRTGWYTNVF |
| | | TLEVGDVENLTCADGPSLIKTELDLTKSAL |
| | | RELKTVSADQLAREEQIENPRQSR |

TABLE 14
Exemplary hMPV A F Protein Mutants Comprising
Engineered Disulfide Mutations and
glycine replacement mutation

| | | Amino Acid Sequence |
|---|---|---|
| | | (residues 103-489 |
| | | for F1 polypeptide |
| Mutant | Muta- | and residues 19-102 |
| ID | tions | for F2 polypeptide) |
| hMPV082 | T411C- | F1 (SEQ ID NO: 79): |
| | Q434C, | FVLGAIALGVATAAAVTAGVAIAKTIRLESE |
| | A140C- | VTAIKNCLKKTNEAVCTLGNGVRVLATAVR |
| | S149C, | ELKDFVSKNLTRAINKNKCDIDDLKMAVSF |
| | G239A | SQFNRRFLNVVRQFSDNAGITPAISLDLMT |
| | | DAELARAVSNMPTSAAQIKLMLENRAMVRR |
| | | KGFGILIGVYGSSVIYMVOLPIFGVIDTPC |
| | | WIVKAAPSCSEKKGNYACLLREDQGWYCQN |
| | | AGSTVYYPNEKDCETRGDHVFCDTAAGINV |
| | | AEQSKECNINISTTNYPCKVSTGRHPISMV |
| | | ALSPLGALVACYKGVSCSIGSNRVGIIKQL |
| | | NKGCSYICNQDADTVTIDNTVYQLSKVEGE |
| | | CHVIKGRPVSSSFDPVKFPEDQFNVALDQV |
| | | FENIENSQALVDQSNRILSSAEKGNT |
| | | F2 (SEQ ID NO: 80): |
| | | LKESYLEESCSTITEGYLSVLRTGWYTNVF |
| | | TLEVGDVENLTCADGPSLIKTELDLTKSAL |
| | | RELKTVSADQLAREEQIENPRQSR |
| hMPV088 | T411C- | F1 (SEQ ID NO: 81): |
| | Q434C, | FVLGAIALGVATAAAVTAGVAIAKTIRLES |
| | L141C- | EVTAIKNACKKTNEAVSTLGNGVRVLATCV |
| | A161C, | RELKDFVSKNLTRAINKNKCDIDDLKMAVS |
| | G239A | FSQFNRRFLNVVRQFSDNAGITPAISLDLM |
| | | TDAELARAVSNMPTSAAQIKLMLENRAMVR |
| | | RKGFGILIGVYGSSVIYMVOLPIFGVIDTP |
| | | CWIVKAAPSCSEKKGNYACLLREDQGWYCQ |
| | | NAGSTVYYPNEKDCETRGDHVFCDTAAGIN |
| | | VAEQSKECNINISTTNYPCKVSTGRHPISM |
| | | VALSPLGALVACYKGVSCSIGSNRVGIIKQ |
| | | LNKGCSYICNQDADTVTIDNTVYQLSKVEG |
| | | ECHVIKGRPVSSSFDPVKFPEDQFNVALDQ |
| | | VFENIENSQALVDQSNRILSSAEKGNT |
| | | F2 (SEQ ID NO: 82): |
| | | LKESYLEESCSTITEGYLSVLRTGWYTNVF |
| | | TLEVGDVENLTCADGPSLIKTELDLTKSAL |
| | | RELKTVSADQLAREEQIENPRQSR |

TABLE 15
Exemplary hMPV A F Protein Mutants Comprising
Engineered Disulfide Mutations
and cavity filling mutations

| | | Amino Acid Sequence |
|---|---|---|
| | | (residues 103-489 |
| | | for F1 polypeptide |
| Mutant | Muta- | and residues 19-102 |
| ID | tions | for F2 polypeptide) |
| hMPV080 | T411C- | F1 (SEQ ID NO: 83): |
| | Q434C, | FVLGAIALGVATAAAVTAGVAIAKTIRLES |
| | A140C- | EVTAIKNCLKKTNEAVCTLGNGVRVLATAV |
| | S149C, | RELKDFVSKNLTRAINKNKCDIDDLKMAVS |
| | T491 | FSQFNRRFLNVVRQFSDNAGITPAISLDLM |
| | | TDAELARAVSNMPTSAGQIKLMLENRAMVR |
| | | RKGFGILIGVYGSSVIYMVQLPIFGVIDTP |
| | | CWIVKAAPSCSEKKGNYACLLREDQGWYCQ |
| | | NAGSTVYYPNEKDCETRGDHVFCDTAAGIN |
| | | VAEQSKECNINISTTNYPCKVSTGRHPISM |
| | | VALSPLGALVACYKGVSCSIGSNRVGIIKQ |
| | | LNKGCSYICNQDADTVTIDNTVYQLSKVEG |
| | | ECHVIKGRPVSSSFDPVKFPEDQFNVALDQ |
| | | VFENIENSQALVDQSNRILSSAEKGNT |
| | | F2 (SEQ ID NO: 84): |
| | | LKESYLEESCSTITEGYLSVLRTGWYTNVF |
| | | ILEVGDVENLTCADGPSLIKTELDLTKSAL |
| | | RELKTVSADQLAREEQIENPRQSR |
| hMPV081 | T411C- | F1 (SEQ ID NO: 85): |
| | Q434C, | FVLGAIALGVATAAAVTAGVAIAKTIRLES |
| | A140C- | EVTAIKNCLKKTNEAVCTLGNGVRVLATAV |
| | S149C, | RELKDFVSKNLTRAINKNKCDIDDLKMAVS |
| | T3651 | FSQFNRRFLNVVRQFSDNAGITPAISLDLM |
| | | TDAELARAVSNMPTSAGQIKLMLENRAMVR |
| | | RKGFGILIGVYGSSVIYMVQLPIFGVIDTP |
| | | CWIVKAAPSCSEKKGNYACLLREDQGWYCQ |
| | | NAGSTVYYPNEKDCETRGDHVFCDTAAGIN |
| | | VAEQSKECNINISTTNYPCKVSIGRHPISM |
| | | VALSPLGALVACYKGVSCSIGSNRVGIIKQ |
| | | LNKGCSYICNQDADTVTIDNTVYQLSKVEG |
| | | ECHVIKGRPVSSSFDPVKFPEDQFNVALDQ |
| | | VFENIENSQALVDQSNRILSSAEKGNT |
| | | F2 (SEQ ID NO: 86): |
| | | LKESYLEESCSTITEGYLSVLRTGWYTNVF |
| | | TLEVGDVENLTCADGPSLIKTELDLTKSAL |
| | | RELKTVSADQLAREEQIENPRQSR |
| hMP0086 | T411C- | F1 (SEQ ID NO: 87): |
| | Q434C, | FVLGAIALGVATAAAVTAGVAIAKTIRLES |
| | L141C- | EVTAIKNACKKTNEAVSTLGNGVRVLATCV |
| | A161C, | RELKDFVSKNLTRAINKNKCDIDDLKMAVS |
| | T491 | FSQFNRRFLNVVRQFSDNAGITPAISLDLM |
| | | TDAELARAVSNMPTSAGQIKLMLENRAMVR |
| | | RKGFGILIGVYGSSVIYMVQLPIFGVIDTP |
| | | CWIVKAAPSCSEKKGNYACLLREDQGWYCQ |
| | | NAGSTVYYPNEKDCETRGDHVFCDTAAGIN |
| | | VAEQSKECNINISTTNYPCKVSTGRHPISM |
| | | VALSPLGALVACYKGVSCSIGSNRVGIIKQ |
| | | LNKGCSYICNQDADTVTIDNTVYQLSKVEG |
| | | ECHVIKGRPVSSSFDPVKFPEDQFNVALDQ |
| | | VFENIENSQALVDQSNRILSSAEKGNT |
| | | F2 (SEQ ID NO: 88): |
| | | LKESYLEESCSTITEGYLSVLRTGWYTNVF |
| | | ILEVGDVENLTCADGPSLIKTELDLTKSAL |
| | | RELKTVSADQLAREEQIENPRQSR |
| hMPV087 | T411C- | F1 (SEQ ID NO: 89): |
| | Q434C, | FVLGAIALGVATAAAVTAGVAIAKTIRLES |
| | L141C- | EVTAIKNACKKTNEAVSTLGNGVRVLATCV |
| | A161C, | RELKDFVSKNLTRAINKNKCDIDDLKMAVS |
| | T3651 | FSQFNRRFLNVVRQFSDNAGITPAISLDLM |
| | | TDAELARAVSNMPTSAGQIKLMLENRAMVR |
| | | RKGFGILIGVYGSSVIYMVQLPIFGVIDTP |
| | | CWIVKAAPSCSEKKGNYACLLREDQGWYCQ |
| | | NAGSTVYYPNEKDCETRGDHVFCDTAAGIN |
| | | VAEQSKECNINISTTNYPCKVSIGRHPISM |
| | | VALSPLGALVACYKGVSCSIGSNRVGIIKQ |
| | | LNKGCSYICNQDADTVTIDNTVYQLSKVEG |
| | | ECHVIKGRPVSSSFDPVKFPEDQFNVALDQ |
| | | VFENIENSQALVDQSNRILSSAEKGNT |
| | | F2 (SEQ ID NO: 90): |
| | | LKESYLEESCSTITEGYLSVLRTGWYTNVF |
| | | TLEVGDVENLTCADGPSLIKTELDLTKSAL |
| | | RELKTVSADQLAREEQIENPRQSR |
| hMPV089 | T411C- | F1 (SEQ ID NO: 91): |
| | Q434C, | FVLGAIALGVATAAAVTAGVAIAKTIRLES |
| | L141C- | EVTAIKNACKKTNEAVTTLGNGVRVLATCV |
| | A161C, | RELKDFVSKNLTRAINKNKCDIDDLKMAVS |
| | S149T | FSQFNRRFLNVVRQFSDNAGITPAISLDLM |
| | | TDAELARAVSNMPTSAGQIKLMLENRAMVR |
| | | RKGFGILIGVYGSSVIYMVQLPIFGVIDTP |
| | | CWIVKAAPSCSEKKGNYACLLREDQGWYCQ |
| | | NAGSTVYYPNEKDCETRGDHVFCDTAAGIN |
| | | VAEQSKECNINISTTNYPCKVSTGRHPISM |
| | | VALSPLGALVACYKGVSCSIGSNRVGIIKQ |
| | | LNKGCSYICNQDADTVTIDNTVYQLSKVEG |
| | | ECHVIKGRPVSSSFDPVKFPEDQFNVALDQ |
| | | VFENIENSQALVDQSNRILSSAEKGNT |
| | | F2 (SEQ ID NO: 92): |
| | | LKESYLEESCSTITEGYLSVLRTGWYTNVF |
| | | TLEVGDVENLTCADGPSLIKTELDLTKSAL |
| | | RELKTVSADQLAREEQIENPRQSR |

TABLE 16
Exemplary hMPV A F Protein Mutants Comprising
Engineered Disulfide Mutations proline
substitution mutations, glycine replacement
mutation, and cavity filling mutations

| | | Amino Acid Sequence |
|---|---|---|
| | | (residues 103-489 |
| | | for F1 polypeptide |
| Mutant | Muta- | and residues 19-102 |
| ID | tions | for F2 polypeptide) |
| hMPV083 | T411C- | F1 (SEQ ID NO: 93): |
| | Q434C, | FVLGAIALGVATAAAVTAGVAIAKT |
| | A140C- | IRLESEVTAIKNCLKKTNEAVCTLG |
| | S149C, | NGVRVLATAVRELKDFVSKNLTRAI |
| | A459P, | NKNKCDIDDLKMAVSFSQFNRRFLN |
| | G239A | VVRQFSDNAGITPAISLDLMTDAEL |
| | T491, | ARAVSNMPTSAAQIKLMLENRAMVR |
| | T3651 | RKGFGILIGVYGSSVIYMVOLPIFG |
| | | VIDTPCWIVKAAPSCSEKKGNYACL |
| | | LREDQGWYCQNAGSTVYYPNEKDCE |
| | | TRGDHVFCDTAAGINVAEQSKECNI |
| | | NISTTNYPCKVSIGRHPISMVALSP |
| | | LGALVACYKGVSCSIGSNRVGIIKQ |
| | | LNKGCSYICNQDADTVTIDNTVYQL |
| | | SKVEGECHVIKGRPVSSSFDPVKFP |
| | | EDQFNVPLDQVFENIENSQALVDQS |
| | | NRILSSAEKGNT |
| | | F2 (SEQ ID NO: 94): |
| | | LKESYLEESCSTITEGYLSVLRTGW |
| | | YTNVFILEVGDVENLTCADGPSLIK |
| | | TELDLTKSALRELKTVSADQLAREE |
| | | QIENPRQSR |
| hMPV090 | T411C- | F1 (SEQ ID NO: 95): |
| | Q434C, | FVLGAIALGVATAAAVTAGVAIAKT |
| | L141C- | IRLESEVTAIKNACKKTNEAVTTLG |
| | A161C, | NGVRVLATCVRELKDFVSKNLTRAI |
| | A459P, | NKNKCDIDDLKMAVSFSQFNRRFLN |
| | G239A | VVRQFSDNAGITPAISLDLMTDAEL |
| | T491, | ARAVSNMPTSAAQIKLMLENRAMVR |
| | S149T, | RKGFGILIGVYGSSVIYMVQLPIFG |
| | T3651 | VIDTPCWIVKAAPSCSEKKGNYACL |
| | | LREDQGWYCQNAGSTVYYPNEKDCE |
| | | TRGDHVFCDTAAGINVAEQSKECNI |
| | | NISTTNYPCKVSIGRHPISMVALSP |
| | | LGALVACYKGVSCSIGSNRVGIIKQ |
| | | LNKGCSYICNQDADTVTIDNTVYQL |
| | | SKVEGECHVIKGRPVSSSFDPVKFP |
| | | EDQFNVPLDQVFENIENSQALVDQS |
| | | NRILSSAEKGNT |
| | | F2 (SEQ ID NO: 96): |
| | | LKESYLEESCSTITEGYLSVLRTGW |
| | | YTNVFILEVGDVENLTCADGPSLIK |
| | | TELDLTKSALRELKTVSADQLAREE |
| | | QIENPRQSR |
| hMPV097 | T411C- | F1 (SEQ ID NO: 97): |
| | Q434C, | FVLGAIALGVATAAAVTAGVAIAKT |
| | E146C- | IRLESEVTAIKNALKKTNCAVTTLG |
| | T160C, | NGVRVLACAVRELKDFVSKNLTRAI |
| | A459P, | NKNKCDIDDLKMAVSFSQFNRRFLN |
| | G239A | VVRQFSDNAGITPAISLDLMTDAEL |
| | T491, | ARAVSNMPTSAAQIKLMLENRAMVR |
| | S149T, | RKGFGILIGVYGSSVIYMVQLPIFG |
| | T3651 | VIDTPCWIVKAAPSCSEKKGNYACL |
| | | LREDQGWYCQNAGSTVYYPNEKDCE |
| | | TRGDHVFCDTAAGINVAEQSKECNI |
| | | NISTTNYPCKVSIGRHPISMVALSP |
| | | LGALVACYKGVSCSIGSNRVGIIKQ |
| | | LNKGCSYICNQDADTVTIDNTVYQL |
| | | SKVEGECHVIKGRPVSSSFDPVKFP |
| | | EDQFNVPLDQVFENIENSQALVDQS |
| | | NRILSSAEKGNT |
| | | F2 (SEQ ID NO: 98): |
| | | LKESYLEESCSTITEGYLSVLRTGW |
| | | YTNVFILEVGDVENLTCADGPSLIK |
| | | TELDLTKSALRELKTVSADQLAREE |
| | | QIENPRQSR |

### Example 2. hMPV a F Protein Mutant Expression Vector Construction

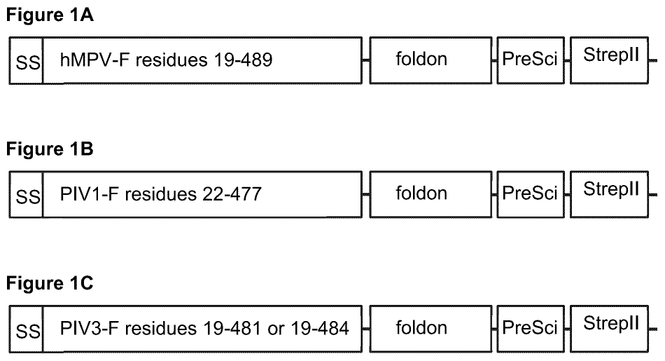

A nucleic acid molecule encoding the truncated hMPV A (strain TN/95/3-54) F0 polypeptide set forth in SEQ ID NO: 128 was mutated using standard molecular biology techniques to encode a precursor polypeptide for a hMPV A F protein mutant having the introduced amino acid mutations disclosed in example 1. The structure and components of the precursor polypeptide are set forth in FIG. 1 and SEQ ID NO: 128. The precursor polypeptide comprises of a signal peptide (residues 1-18), F2 polypeptide (residues 19-102), F1 polypeptide (residues 103-489), T4 fibritin foldon (residues 494-520), PreScission cleavage site (residues 524-531), Strep Tag II (residues 535-542), and linker sequences (residues 490-493, 521-523, 532-534).

The protein sequences of SEQ ID NO: 128 was submitted for mammalian codon optimization by Genscript (Piscataway, NJ). The nucleotide sequence was introduced into a commercially available expression vector, pcDNA3.1/Zeo(+) (ThermoFisher Scientific, Waltham, MA) that has been modified to encode the CAG promoter (Yamamura et al., Gene, 108(2), 193-199, 1991) in place of the CMV promoter. Double stranded DNA fragments were purchased from Integrated DNA Technologies (Coralville, IA). DNA fragments of the mutagenized F allele not synthesized were generated and amplified by polymerase chain reaction (PCR) with Phusion Flash High-Fidelity PCR Master Mix (ThermoFisher Scientific). Following purification of the linearized expression vector digested using BamHI and Notl, gene fragments of the mutagenized F allele were inserted into the expression vector with NEBuilder HiFi DNA Assembly Kit (New England Biolabs, Ipswich, MA). The presence of the intended sequence was confirmed by Sanger DNA sequencing. Plasmid DNA for transfection into ExpiCHO cells was purified with the Qiagen Plasmid Plus Midi Kit (Qiagen, Valencia, CA). For all commercial kits or reagents, procedures were performed according to the manufacturer's protocol.

### Example 3. Transfection of hMPV a F Protein Mutants

Proteins for hMPV A F mutant evaluation were produced by transient transfection of ExpiCHO cells (ThermoFisher Scientific) with DNA plasmids assembled and prepared as described in Example 2. Transient transfections were carried out according to the manufacturer's protocol. On day 5 post transfection, the cultures were centrifuged, and supernatants were separated from cell pellets. The crude cell supernatants were used for in vitro assays described herein.

### Example 4. Expression and Conformational Integrity of hMPV a F Protein Mutants

The OCTET HTX (Sartorius, Gottingen, Germany) instrument was used to evaluate the expression and conformational integrity for each mutant. All measurements were conducted at 30° C. temperature in 96-well black plates (Corning, Corning, NY) with a final volume of 240 μL per well at a constant agitation rate of 1000 RPM.

4A: Quantitation of Expression of hMPV A F Proteins Mutants

Crude cell supernatant was used to quantitate the expression levels of hMPV A F protein mutants. Anti-Murine IgG Quantitation (AMQ) Biosensors (Sartorius) were first equilibrated in phosphate-buffered saline (PBS), 1% bovine serum albumin (BSA) (PB) before being dipped into more PB to establish the initial baseline. Biosensors were incubated with a mouse Strep-Tag® II monoclonal antibody (mAb) (Novagen, EMD Millipore Corporation, Temecula, CA) before being equilibrated in PB to establish the experimental baseline. The mAb-bound biosensors were then dipped into crude cell culture supernatant for 2.5 minutes. OCTET data analysis software (version 12.0, Sartorius) was used to generate a standard curve from a serially diluted purified protein reference within the same assay. Titers for protein mutants were then determined based on the standard curve.

The results are presented in Table 17 and Table 17.1. The highest expressing amino acid substitutions identified from the screens of the individual engineered disulfide mutants, proline mutations, glycine replacement mutations and cavity filling mutants were combined into the combination mutants. The protein expression of combination mutants was measured using the same procedure as described above. The results are presented in Table 18.

TABLE 17
Protein expression and thermal stability of hMPV A F protein mutants.

| | | | Response Shift with | | | Response Shift with | | | |
|---|---|---|---|---|---|---|---|---|---|
| | Protein | | MPE8 mAb | | | hMPV-2 mAb | | | |
| | expression | | | 50° C. | 53° C. | | 50° C. | 53° C. | 50° C. |
| | | Fold | No | stress | stress | No | stress | stress | Fold |
| | | change | stress | resistance | resistance | stress | resistance | resistance | change |
| Mutant ID | mg/L | vs WT | (nm) | (nm) | (nm) | (nm) | (nm) | (nm) | vs WT |
| WT Control | 31.2 | 1.0 | 0.690 | 0.174 | −0.017 | 0.782 | 0.284 | −0.026 | 1.0 |
| hMPV043 | 2.22 | 0.1 | 0.558 | 0.501 | 0.010 | 0.636 | 0.567 | 0.030 | 2.0 |
| hMPV044 | 21.4 | 0.7 | 0.720 | 0.722 | 0.534 | 0.797 | 0.737 | 0.618 | 2.6 |
| hMPV045 | 61.1 | 2.0 | 0.603 | 0.139 | −0.025 | 0.582 | 0.124 | −0.048 | 0.4 |
| hMPV046 | 102.8 | 3.3 | 0.603 | 0.412 | −0.012 | 0.573 | 0.472 | 0.196 | 1.7 |
| hMPV047 | 44.2 | 1.4 | 0.259 | −0.024 | −0.051 | 0.268 | −0.039 | −0.079 | −0.1 |
| hMPV048 | 78.3 | 2.5 | 0.596 | 0.377 | −0.056 | 0.585 | 0.439 | −0.059 | 1.5 |
| hMPV049 | 63.4 | 2.0 | 0.672 | 0.343 | −0.065 | 0.700 | 0.549 | −0.011 | 1.9 |
| hMPV050 | 66.1 | 2.1 | 0.641 | 0.140 | −0.014 | 0.669 | 0.142 | −0.032 | 0.5 |
| hMPV052 | 12.7 | 0.4 | 0.716 | 0.482 | −0.045 | 0.739 | 0.634 | −0.044 | 2.2 |
| hMPV053 | 46.2 | 1.5 | 0.624 | 0.422 | −0.032 | 0.717 | 0.566 | 0.017 | 2.0 |
| hMPV054 | 25.9 | 0.8 | 0.730 | 0.381 | −0.064 | 0.682 | 0.467 | −0.072 | 1.6 |
| hMPV055 | 35.4 | 1.1 | 0.581 | 0.098 | −0.078 | 0.627 | 0.194 | −0.073 | 0.7 |
| hMPV058 | 20.2 | 0.6 | 0.684 | 0.403 | −0.050 | 0.709 | 0.494 | −0.052 | 1.7 |
| hMPV059 | 3.1 | 0.1 | 0.666 | 0.344 | −0.039 | 0.719 | 0.455 | −0.051 | 1.6 |
| hMPV060 | 11.3 | 0.4 | 0.840 | 0.712 | −0.060 | 0.769 | 0.707 | −0.045 | 2.5 |
| hMPV061 | 3.4 | 0.1 | 0.813 | 0.537 | −0.059 | 0.730 | 0.561 | −0.073 | 2.0 |
| hMPV062 | 13.1 | 0.4 | 0.713 | 0.253 | 0.006 | 0.774 | 0.460 | 0.013 | 1.6 |
| hMPV063 | 17.1 | 0.5 | 0.849 | 0.570 | −0.008 | 0.854 | 0.685 | 0.001 | 2.4 |
| hMPV066 | 16.7 | 0.5 | 0.779 | 0.666 | −0.003 | 0.767 | 0.738 | 0.265 | 2.6 |
| hMPV067 | 18.1 | 0.6 | 0.744 | 0.571 | −0.041 | 0.677 | 0.654 | 0.184 | 2.3 |
| hMPV068 | 14.4 | 0.5 | 0.738 | 0.194 | 0.002 | 0.783 | 0.441 | 0.022 | 1.6 |
| hMPV069 | 8.3 | 0.3 | 0.651 | 0.355 | −0.003 | 0.747 | 0.465 | −0.005 | 1.6 |
| hMPV071 | 13.2 | 0.4 | 0.804 | 0.671 | −0.039 | 0.760 | 0.694 | −0.046 | 2.4 |
| hMPV075 | 9.7 | 0.3 | 0.777 | 0.506 | −0.069 | 0.802 | 0.653 | −0.082 | 2.3 |
| hMPV077 | 4.0 | 0.1 | 0.706 | 0.472 | −0.041 | 0.751 | 0.635 | −0.007 | 2.2 |

TABLE 17.1
Protein expression and thermal stability of hMPV A F protein
mutants comprising engineered disulfide mutations and cavity
filling mutations with alternative thermal stress conditions.

| | Protein | Response Shift with MPE8 mAb | | |
|---|---|---|---|---|
| | expression | No stress | 53° C. stress | 68° C. stress |
| Mutant ID | (mg/L) | (nm) | resistance (nm) | resistance (nm) |
| WT Control | 11.4 | 0.102 | 0.037 | 0.025 |
| hMPV080 | 19.2 | 0.787 | 0.798 | −0.075 |

4B: Conformational Integrity of hMPV A F Protein Mutants

Conformation integrity of hMPV A F protein mutants were evaluated by thermal stress experiments. Crude cell culture supernatants were normalized based on the results of the titer quantitation and then incubated for 1 hour at room temperature and two higher temperatures. For the initial testing of hMPV A F protein mutants, the two temperatures were 50° C. and 53° C., as shown in Table 17. Some additional mutants were stressed at higher temperature, 53° C., and 68° C., as shown in Table 17.1. After the incubation, the protein mutants were probed with prefusion specific monoclonal antibody MPE8 (Corti et. al. Nature. 2013; 501:439-443) and hMPV-2 mAb by OCTET HTX. Anti-Human IgG Fc Capture (AHC) biosensors were first equilibrated in PB before being dipped into more PB to establish the initial baseline. Biosensors were incubated with MPE8 or hMPV-2 mAb before being equilibrated in PB to establish the experimental baseline. The mAb-bound biosensors were then dipped into the thermal stressed cell culture supernatants for 5 minutes. The kinetics analysis was done by OCTET data analysis software (version 12.0, Sartorius) based on curve fitting of the entire associate step.

The reactivities of the mutants to MPE8 and hMPV-2 mAb are presented in Table 17 and 17.1. The most stabilizing amino acid substitutions identified from the screens of the individual engineered disulfide mutants, proline mutations, glycine replacement mutations and cavity filling mutants were combined into the combination mutants. The combination mutants were subjected to the thermal stress test at room temperature, 53° C., and 58° C., as shown in Table 18. Notable protein expression and thermal stability improvement were observed for combination mutants.

TABLE 18
Protein expression and thermal stability of hMPV A F protein mutants
comprising engineered disulfide mutations, proline substitution mutations,
glycine replacement mutations, and cavity filling mutations.

| | | Response Shift with | | | Response Shift with | | |
|---|---|---|---|---|---|---|---|
| | | MPE8 mAb | | | hMPV-2 mAb | | |
| | | | 53° C. | 58° C. | | 53° C. | 58° C. |
| | Protein | No | stress | stress | No | stress | stress |
| | expression | stress | resistance | resistance | stress | resistance | resistance |
| Mutant ID | (mg/L) | (nm) | (nm) | (nm) | (nm) | (nm) | (nm) |
| WT Control | 2.9 | 0.926 | 0.096 | 0.006 | 0.898 | −0.014 | 0.015 |
| hMPV078 | 2.1 | 0.955 | 0.939 | 0.939 | 0.860 | 0.757 | 0.769 |
| hMPV079 | 6.7 | 0.887 | 0.861 | 0.867 | 0.791 | 0.730 | 0.736 |
| hMPV081 | 4.9 | 0.955 | 0.915 | 0.945 | 0.837 | 0.778 | 0.804 |
| hMPV082 | 11.3 | 0.949 | 0.985 | 0.928 | 0.854 | 0.816 | 0.804 |
| hMPV083 | 19.0 | 0.805 | 0.796 | 0.768 | 0.847 | 0.755 | 0.822 |
| hMPV084 | 1.6 | 0.924 | 0.908 | 0.665 | 0.862 | 0.776 | 0.183 |
| hMPV085 | 1.9 | 0.877 | 0.889 | 0.757 | 0.766 | 0.672 | 0.076 |
| hMPV086 | 4.7 | 0.834 | 0.860 | 0.736 | 0.851 | 0.746 | 0.735 |
| hMPV087 | 4.2 | 0.979 | 0.903 | 0.805 | 0.867 | 0.709 | 0.763 |
| hMPV088 | 4.7 | 0.974 | 0.937 | 0.812 | 0.878 | 0.816 | 0.492 |
| hMPV089 | 1.6 | 0.959 | 0.928 | 0.917 | 0.891 | 0.801 | N/A |
| hMPV090 | 1.6 | 0.903 | 0.909 | 0.906 | 0.895 | 0.815 | 0.770 |
| hMPV097 | 4.0 | 0.918 | 0.935 | 0.918 | 0.923 | 0.820 | 0.905 |

### Example 5: Design and Preparation of Hmpv B F Protein Mutants

This example illustrates the design and preparation of various hMPV B F protein mutants, which include a fibritin foldon trimerization domain and introduced amino acid mutations, such as engineered disulfide bond mutations, cavity-filling mutations, proline substitution mutations, and glycine replacement mutations, or a combination thereof. Exemplary hMPV B F protein mutants, each of which is identified by a unique identifier, such as hMPV117, hMPV118 etc., are provided in Tables 19-29. Each of these mutants was designed and prepared based on the amino acid sequence set forth in SEQ ID NO: 129. Amino acid residues 1-489 of the sequence of SEQ ID NO:129 are identical to amino acid residues 1-489 of the F0 precursor polypeptide of consensus hMPV B as set forth in SEQ ID NO. Therefore, the amino acid sequences of these exemplary F protein mutants are identical except for the introduced amino acid mutations as noted for each mutant listed in Tables 19-29. Each of these hMPV B F protein mutants comprises two separate polypeptide chains. One of the polypeptide chains, the F2 polypeptide, comprises amino acids 19-102 of SEQ ID NO: 129 except for the introduced mutations as noted. The other polypeptide chain comprises the F1 polypeptide (residues 103-489) linked to a foldon trimerization domain (residues 494-520) via a GGGS linker (residues 490-493). The signal peptide (residues 1-18) of SEQ ID NO: 129 were cleaved from the F0 precursor during the expression process.

TABLE 19
Exemplary hMPV B F Protein Mutants
Comprising Engineered Disulfide

| | | Amino Acid Sequence (residues |
|---|---|---|
| | | 103-489 for F1 polypeptide |
| Mutant | Muta- | and residues 19-102 |
| ID | tions | for F2 polypeptide) |
| hMPV100 | T411C- | F1 (SEQ ID NO: 130): |
| | Q434C | FVLGAIALGVATAAAVTAGIAIAKTIRLES |
| | | EVNAIKGALKTTNEAVSTLGNGVRVLATAV |
| | | RELKEFVSKNLTSAINKNKCDIADLKMAVS |
| | | FSQFNRRFLNVVRQFSDNAGITPAISLDLM |
| | | NDAELARAVSYMPTSAGQIKLMLENRAMVR |
| | | RKGFGILIGVYGSSVIYMVQLPIFGVINTP |
| | | CWIIKAAPSCSEKDGNYACLLREDQGWYCK |
| | | NAGSTVYYPNEKDCETRGDHVFCDTAAGIN |
| | | VAEQSRECNINISTTNYPCKVSTGRHPISM |
| | | VALSPLGALVACYKGVSCSIGSNQVGIIKQ |
| | | LPKGCSYICNQDADTVTIDNTVYQLSKVEG |
| | | ECHVIKGRPVSSSFDPIRFPEDQFNVALDQ |
| | | VFESIENSQALVDQSNKILNSAEKGNT |
| | | F2 (SEQ ID NO: 131): |
| | | LKESYLEESCSTITEGYLSVLRTGWYTNVF |
| | | TLEVGDVENLTCTDGPSLIKTELDLTKSAL |
| | | RELKTVSADQLAREEQIENPRQSR |
| hMPV101 | A140C- | F1 (SEQ ID NO: 132): |
| | S149C | FVLGAIALGVATAAAVTAGIAIAKTIRLES |
| | | EVNAIKGCLKTTNEAVCTLGNGVRVLATAV |
| | | RELKEFVSKNLTSAINKNKCDIADLKMAVS |
| | | FSQFNRRFLNVVRQFSDNAGITPAISLDLM |
| | | NDAELARAVSYMPTSAGQIKLMLENRAMVR |
| | | RKGFGILIGVYGSSVIYMVQLPIFGVINTP |
| | | CWIIKAAPSCSEKDGNYACLLREDQGWYCK |
| | | NAGSTVYYPNEKDCETRGDHVFCDTAAGIN |
| | | VAEQSRECNINISTTNYPCKVSTGRHPISM |
| | | VALSPLGALVACYKGVSCSIGSNQVGIIKQ |
| | | LPKGCSYITNQDADTVTIDNTVYQLSKVEG |
| | | EQHVIKGRPVSSSFDPIRFPEDQFNVALDQ |
| | | VFESIENSQALVDQSNKILNSAEKGNT |
| | | F2 (SEQ ID NO: 133): |
| | | LKESYLEESCSTITEGYLSVLRTGWYTNVF |
| | | TLEVGDVENLTCTDGPSLIKTELDLTKSAL |
| | | RELKTVSADQLAREEQIENPRQSR |
| hMPV102 | L141C- | F1 (SEQ ID NO: 134): |
| | A161C | FVLGAIALGVATAAAVTAGIAIAKTIRLES |
| | | EVNAIKGACKTTNEAVSTLGNGVRVLATCV |
| | | RELKEFVSKNLTSAINKNKCDIADLKMAVS |
| | | FSQFNRRFLNVVRQFSDNAGITPAISLDLM |
| | | NDAELARAVSYMPTSAGQIKLMLENRAMVR |
| | | RKGFGILIGVYGSSVIYMVQLPIFGVINTP |
| | | CWIIKAAPSCSEKDGNYACLLREDQGWYCK |
| | | NAGSTVYYPNEKDCETRGDHVFCDTAAGIN |
| | | VAEQSRECNINISTTNYPCKVSTGRHPISM |
| | | VALSPLGALVACYKGVSCSIGSNQVGIIKQ |
| | | LPKGCSYITNQDADTVTIDNTVYQLSKVEG |
| | | EQHVIKGRPVSSSFDPIRFPEDQFNVALDQ |
| | | VFESIENSQALVDQSNKILNSAEKGNT |
| | | F2 (SEQ ID NO: 135): |
| | | LKESYLEESCSTITEGYLSVLRTGWYTNVF |
| | | TLEVGDVENLTCTDGPSLIKTELDLTKSAL |
| | | RELKTVSADQLAREEQIENPRQSR |
| hMPV103 | E146C- | F1 (SEQ ID NO: 136): |
| | T160C | FVLGAIALGVATAAAVTAGIAIAKTIRLES |
| | | EVNAIKGALKTTNCAVSTLGNGVRVLACAV |
| | | RELKEFVSKNLTSAINKNKCDIADLKMAVS |
| | | FSQFNRRFLNVVRQFSDNAGITPAISLDLM |
| | | NDAELARAVSYMPTSAGQIKLMLENRAMVR |
| | | RKGFGILIGVYGSSVIYMVQLPIFGVINTP |
| | | CWIIKAAPSCSEKDGNYACLLREDQGWYCK |
| | | NAGSTVYYPNEKDCETRGDHVFCDTAAGIN |
| | | VAEQSRECNINISTTNYPCKVSTGRHPISM |
| | | VALSPLGALVACYKGVSCSIGSNQVGIIKQ |
| | | LPKGCSYITNQDADTVTIDNTVYQLSKVEG |
| | | EQHVIKGRPVSSSFDPIRFPEDQFNVALDQ |
| | | VFESIENSQALVDQSNKILNSAEKGNT |
| | | F2 (SEQ ID NO: 137): |
| | | LKESYLEESCSTITEGYLSVLRTGWYTNVF |
| | | TLEVGDVENLTCTDGPSLIKTELDLTKSAL |
| | | RELKTVSADQLAREEQIENPRQSR |
| hMPV116 | T150C- | F1 (SEQ ID NO: 138): |
| | R156C | FVLGAIALGVATAAAVTAGIAIAKTIRLES |
| | | EVNAIKGALKTTNEAVSCLGNGVCVLATAV |
| | | RELKEFVSKNLTSAINKNKCDIADLKMAVS |
| | | FSQFNRRFLNVVRQFSDNAGITPAISLDLM |
| | | NDAELARAVSYMPTSAGQIKLMLENRAMVR |
| | | RKGFGILIGVYGSSVIYMVQLPIFGVINTP |
| | | CWIIKAAPSCSEKDGNYACLLREDQGWYCK |
| | | NAGSTVYYPNEKDCETRGDHVFCDTAAGIN |
| | | VAEQSRECNINISTTNYPCKVSTGRHPISM |
| | | VALSPLGALVACYKGVSCSIGSNQVGIIKQ |
| | | LPKGCSYITNQDADTVTIDNTVYQLSKVEG |
| | | EQHVIKGRPVSSSFDPIRFPEDQFNVALDQ |
| | | VFESIENSQALVDQSNKILNSAEKGNT |
| | | F2 (SEQ ID NO: 139): |
| | | LKESYLEESCSTITEGYLSVLRTGWYTNVF |
| | | TLEVGDVENLTCTDGPSLIKTELDLTKSAL |
| | | RELKTVSADQLAREEQIENPRQSR |
| hMPV120 | A140C- | F1 (SEQ ID NO: 140): |
| | S149C, | FVLGAIALGVATAAAVTAGIAIAKTIRLES |
| | T411C- | EVNAIKGCLKTTNEAVCTLGNGVRVLATAV |
| | Q434C | RELKEFVSKNLTSAINKNKCDIADLKMAVS |
| | | FSQFNRRFLNVVRQFSDNAGITPAISLDLM |
| | | NDAELARAVSYMPTSAGQIKLMLENRAMVR |
| | | RKGFGILIGVYGSSVIYMVQLPIFGVINTP |
| | | CWIIKAAPSCSEKDGNYACLLREDQGWYCK |
| | | NAGSTVYYPNEKDCETRGDHVFCDTAAGIN |
| | | VAEQSRECNINISTTNYPCKVSTGRHPISM |
| | | VALSPLGALVACYKGVSCSIGSNQVGIIKQ |
| | | LPKGCSYICNQDADTVTIDNTVYQLSKVEG |
| | | ECHVIKGRPVSSSFDPIRFPEDQFNVALDQ |
| | | VFESIENSQALVDQSNKILNSAEKGNT |
| | | F2 (SEQ ID NO: 141): |
| | | LKESYLEESCSTITEGYLSVLRTGWYTNVF |
| | | TLEVGDVENLTCTDGPSLIKTELDLTKSAL |
| | | RELKTVSADQLAREEQIENPRQSR |
| hMPV121 | L141C- | F1 (SEQ ID NO: 142): |
| | A161C, | FVLGAIALGVATAAAVTAGIAIAKTIRLES |
| | T411C- | EVNAIKGACKTTNEAVSTLGNGVRVLATCV |
| | Q434C | RELKEFVSKNLTSAINKNKCDIADLKMAVS |
| | | FSQFNRRFLNVVRQFSDNAGITPAISLDLM |
| | | NDAELARAVSYMPTSAGQIKLMLENRAMVR |
| | | RKGFGILIGVYGSSVIYMVQLPIFGVINTP |
| | | CWIIKAAPSCSEKDGNYACLLREDQGWYCK |
| | | NAGSTVYYPNEKDCETRGDHVFCDTAAGIN |
| | | VAEQSRECNINISTTNYPCKVSTGRHPISM |
| | | VALSPLGALVACYKGVSCSIGSNQVGIIKQ |
| | | LPKGCSYICNQDADTVTIDNTVYQLSKVEG |
| | | ECHVIKGRPVSSSFDPIRFPEDQFNVALDQ |
| | | VFESIENSQALVDQSNKILNSAEKGNT |
| | | F2 (SEQ ID NO: 143): |
| | | LKESYLEESCSTITEGYLSVLRTGWYTNVF |
| | | TLEVGDVENLTCTDGPSLIKTELDLTKSAL |
| | | RELKTVSADQLAREEQIENPRQSR |

TABLE 21
Exemplary hMPV B F Protein Mutants Comprising
glycine replacement mutation

| | | Amino Acid Sequence (residues |
|---|---|---|
| | | 103-489 for F1 polypeptide and |
| Mutant | Muta- | residues 19-102 for F2 |
| ID | tions | polypeptide) |
| hMPV105 | G239A | F1 (SEQ ID NO: 146): |
| | | FVLGAIALGVATAAAVTAGIAIAKTIRLESEVNAI |
| | | KGALKTTNEAVSTLGNGVRVLATAVRELKEFVSKN |
| | | LTSAINKNKCDIADLKMAVSFSQFNRRFLNVVRQF |
| | | SDNAGITPAISLDLMNDAELARAVSYMPTSAAQIK |
| | | LMLENRAMVRRKGFGILIGVYGSSVIYMVQLPIFG |
| | | VINTPCWIIKAAPSCSEKDGNYACLLREDQGWYCK |
| | | NAGSTVYYPNEKDCETRGDHVFCDTAAGINVAEQS |
| | | RECNINISTTNYPCKVSTGRHPISMVALSPLGALV |
| | | ACYKGVSCSIGSNQVGIIKQLPKGCSYITNQDADT |
| | | VTIDNTVYQLSKVEGEQHVIKGRPVSSSFDPIRFP |
| | | EDQFNVALDQVFESIENSQALVDQSNKILNSAEKG |
| | | NT |
| | | F2 (SEQ ID NO: 147): |
| | | LKESYLEESCSTITEGYLSVLRTGWYTNVFTLEVG |
| | | DVENLTCTDGPSLIKTELDLTKSALRELKTVSADQ |
| | | LAREEQIENPRQSR |

TABLE 22
Exemplary hMPV B F Protein Mutants Comprising
Cavity Filling Mutations

| | | Amino Acid Sequence (residues |
|---|---|---|
| | | 103-489 for F1 polypeptide and |
| Mutant | Muta- | residues 19-102 for F2 |
| ID | tions | polypeptide) |
| hMPV | T491 | F1 (SEQ ID NO: 148): |
| 106 | | FVLGAIALGVATAAAVTAGIAIAKTIRLESEVNAI |
| | | KGALKTTNEAVSTLGNGVRVLATAVRELKEFVSKN |
| | | LTSAINKNKCDIADLKMAVSFSQFNRRFLNVVRQF |
| | | SDNAGITPAISLDLMNDAELARAVSYMPTSAGQIK |
| | | LMLENRAMVRRKGFGILIGVYGSSVIYMVQLPIFG |
| | | VINTPCWIIKAAPSCSEKDGNYACLLREDQGWYCK |
| | | NAGSTVYYPNEKDCETRGDHVFCDTAAGINVAEQS |
| | | RECNINISTTNYPCKVSTGRHPISMVALSPLGALV |
| | | ACYKGVSCSIGSNQVGIIKQLPKGCSYITNQDADT |
| | | VTIDNTVYQLSKVEGEQHVIKGRPVSSSFDPIRFP |
| | | EDQFNVALDQVFESIENSQALVDQSNKILNSAEKG |
| | | NT |
| | | F2 (SEQ ID NO: 149): |
| | | LKESYLEESCSTITEGYLSVLRTGWYTNVFILEVG |
| | | DVENLTCTDGPSLIKTELDLTKSALRELKTVSADQ |
| | | LAREEQIENPRQSR |
| hMPV | S149T | F1 (SEQ ID NO: 150): |
| 107 | | FVLGAIALGVATAAAVTAGIAIAKTIRLESEVNAI |
| | | KGALKTTNEAVTTLGNGVRVLATAVRELKEFVSKN |
| | | LTSAINKNKCDIADLKMAVSFSQFNRRFLNVVRQF |
| | | SDNAGITPAISLDLMNDAELARAVSYMPTSAGQIK |
| | | LMLENRAMVRRKGFGILIGVYGSSVIYMVQLPIFG |
| | | VINTPCWIIKAAPSCSEKDGNYACLLREDQGWYCK |
| | | NAGSTVYYPNEKDCETRGDHVFCDTAAGINVAEQS |
| | | RECNINISTTNYPCKVSTGRHPISMVALSPLGALV |
| | | ACYKGVSCSIGSNQVGIIKQLPKGCSYITNQDADT |
| | | VTIDNTVYQLSKVEGEQHVIKGRPVSSSFDPIRFP |
| | | EDQFNVALDQVFESIENSQALVDQSNKILNSAEKG |
| | | NT |
| | | F2 (SEQ ID NO: 151): |
| | | LKESYLEESCSTITEGYLSVLRTGWYTNVFTLEVG |
| | | DVENLTCTDGPSLIKTELDLTKSALRELKTVSADQ |
| | | LAREEQIENPRQSR |
| hMPV | T3651 | F1 (SEQ ID NO: 152): |
| 108 | | FVLGAIALGVATAAAVTAGIAIAKTIRLESEVNAI |
| | | KGALKTTNEAVSTLGNGVRVLATAVRELKEFVSKN |
| | | LTSAINKNKCDIADLKMAVSFSQFNRRFLNVVRQF |
| | | SDNAGITPAISLDLMNDAELARAVSYMPTSAGQIK |
| | | LMLENRAMVRRKGFGILIGVYGSSVIYMVQLPIFG |
| | | VINTPCWIIKAAPSCSEKDGNYACLLREDQGWYCK |
| | | NAGSTVYYPNEKDCETRGDHVFCDTAAGINVAEQS |
| | | RECNINISTTNYPCKVSIGRHPISMVALSPLGALV |
| | | ACYKGVSCSIGSNQVGIIKQLPKGCSYITNQDADT |
| | | VTIDNTVYQLSKVEGEQHVIKGRPVSSSFDPIRFP |
| | | EDQFNVALDQVFESIENSQALVDQSNKILNSAEKG |
| | | NT |
| | | F2 (SEQ ID NO: 153): |
| | | LKESYLEESCSTITEGYLSVLRTGWYTNVFTLEVG |
| | | DVENLTCTDGPSLIKTELDLTKSALRELKTVSADQ |
| | | LAREEQIENPRQSR |

TABLE 22.1
Exemplary hMPV B F Protein Mutants Comprising
proline substitution

| | | Amino Acid Sequence (residues |
|---|---|---|
| | | 103-489 for F1 polypeptide and |
| Mutant | Muta- | residues 19-102 for F2 |
| ID | tions | polypeptide) |
| hMPV104 | A459P | F1 (SEQ ID NO: 388): |
| | | FVLGAIALGVATAAAVTAGIAIAKTIRLESEVNAI |
| | | KGALKTTNEAVSTLGNGVRVLATAVRELKEFVSKN |
| | | LTSAINKNKCDIADLKMAVSFSQFNRRFLNVVRQF |
| | | SDNAGITPAISLDLMNDAELARAVSYMPTSAGQIK |
| | | LMLENRAMVRRKGFGILIGVYGSSVIYMVQLPIFG |
| | | VINTPCWIIKAAPSCSEKDGNYACLLREDQGWYCK |
| | | NAGSTVYYPNEKDCETRGDHVFCDTAAGINVAEQS |
| | | RECNINISTTNYPCKVSTGRHPISMVALSPLGALV |
| | | ACYKGVSCSIGSNQVGIIKQLPKGCSYITNQDADT |
| | | VTIDNTVYQLSKVEGEQHVIKGRPVSSSFDPIRFP |
| | | EDQFNVPLDQVFESIENSQALVDQSNKILNSAEKG |
| | | NT |
| | | F2 (SEQ ID NO: 389): |
| | | LKESYLEESCSTITEGYLSVLRTGWYTNVFTLEVG |
| | | DVENLTCTDGPSLIKTELDLTKSALRELKTVSADQ |
| | | LAREEQIENPRQSR |

TABLE 23
Exemplary hMPV B F Protein Mutants Comprising
Engineered Disulfide Mutations
and proline substitution mutations

| | | Amino Acid Sequence (residues |
|---|---|---|
| | | 103-489 for F1 polypeptide and |
| Mutant | Muta- | residues 19-102 for F2 |
| ID | tions | polypeptide) |
| hMPV | T150C- | F1 (SEQ ID NO: 154): |
| 117 | R156C, | FVLGAIALGVATAAAVTAGIAIAKTIRLESEVNAI |
| | A459P | KGALKTTNEAVSCLGNGVCVLATAVRELKEFVSKN |
| | | LTSAINKNKCDIADLKMAVSFSQFNRRFLNVVRQF |
| | | SDNAGITPAISLDLMNDAELARAVSYMPTSAGQIK |
| | | LMLENRAMVRRKGFGILIGVYGSSVIYMVQLPIFG |
| | | VINTPCWIIKAAPSCSEKDGNYACLLREDQGWYCK |
| | | NAGSTVYYPNEKDCETRGDHVFCDTAAGINVAEQS |
| | | RECNINISTTNYPCKVSTGRHPISMVALSPLGALV |
| | | ACYKGVSCSIGSNQVGIIKQLPKGCSYITNQDADT |
| | | VTIDNTVYQLSKVEGEQHVIKGRPVSSSFDPIRFP |
| | | EDQFNVPLDQVFESIENSQALVDQSNKILNSAEKG |
| | | NT |
| | | F2 (SEQ ID NO: 155): |
| | | LKESYLEESCSTITEGYLSVLRTGWYTNVFTLEVG |
| | | DVENLTCTDGPSLIKTELDLTKSALRELKTVSADQ |
| | | LAREEQIENPRQSR |
| hMPV | A140C- | F1 (SEQ ID NO: 156): |
| 122 | S149C, | FVLGAIALGVATAAAVTAGIAIAKTIRLESEVNAI |
| | T411C- | KGCLKTTNEAVCTLGNGVRVLATAVRELKEFVSKN |
| | Q434C, | LTSAINKNKCDIADLKMAVSFSQFNRRFLNVVRQF |
| | A459P | SDNAGITPAISLDLMNDAELARAVSYMPTSAGQIK |
| | | LMLENRAMVRRKGFGILIGVYGSSVIYMVQLPIFG |
| | | VINTPCWIIKAAPSCSEKDGNYACLLREDQGWYCK |
| | | NAGSTVYYPNEKDCETRGDHVFCDTAAGINVAEQS |
| | | RECNINISTTNYPCKVSTGRHPISMVALSPLGALV |
| | | ACYKGVSCSIGSNQVGIIKQLPKGCSYICNQDADT |
| | | VTIDNTVYQLSKVEGECHVIKGRPVSSSFDPIRFP |
| | | EDQFNVPLDQVFESIENSQALVDQSNKILNSAEKG |
| | | NT |
| | | F2 (SEQ ID NO: 157): |
| | | LKESYLEESCSTITEGYLSVLRTGWYTNVFTLEVG |
| | | DVENLTCTDGPSLIKTELDLTKSALRELKTVSADQ |
| | | LAREEQIENPRQSR |
| hMPV | L141C- | F1 (SEQ ID NO: 158): |
| 124 | A161C, | FVLGAIALGVATAAAVTAGIAIAKTIRLESEVNAI |
| | T411C- | KGACKTTNEAVSTLGNGVRVLATCVRELKEFVSKN |
| | Q434C, | LTSAINKNKCDIADLKMAVSFSQFNRRFLNVVRQF |
| | A459P | SDNAGITPAISLDLMNDAELARAVSYMPTSAGQIK |
| | | LMLENRAMVRRKGFGILIGVYGSSVIYMVQLPIFG |
| | | VINTPCWIIKAAPSCSEKDGNYACLLREDQGWYCK |
| | | NAGSTVYYPNEKDCETRGDHVFCDTAAGINVAEQS |
| | | RECNINISTTNYPCKVSTGRHPISMVALSPLGALV |
| | | ACYKGVSCSIGSNQVGIIKQLPKGCSYICNQDADT |
| | | VTIDNTVYQLSKVEGECHVIKGRPVSSSFDPIRFP |
| | | EDQFNVPLDQVFESIENSQALVDQSNKILNSAEKG |
| | | NT |
| | | F2 (SEQ ID NO: 159): |
| | | LKESYLEESCSTITEGYLSVLRTGWYTNVFTLEVG |
| | | DVENLTCTDGPSLIKTELDLTKSALRELKTVSADQ |
| | | LAREEQIENPRQSR |
| hMPV | A140C- | F1 (SEQ ID NO: 160): |
| 136 | S149C, | FVLGAIALGVATAAAVTAGIAIAKTIRLESEVNAI |
| | A459P | KGCLKTTNEAVCTLGNGVRVLATAVRELKEFVSKN |
| | | LTSAINKNKCDIADLKMAVSFSQFNRRFLNVVRQF |
| | | SDNAGITPAISLDLMNDAELARAVSYMPTSAGQIK |
| | | LMLENRAMVRRKGFGILIGVYGSSVIYMVQLPIFG |
| | | VINTPCWIIKAAPSCSEKDGNYACLLREDQGWYCK |
| | | NAGSTVYYPNEKDCETRGDHVFCDTAAGINVAEQS |
| | | RECNINISTTNYPCKVSTGRHPISMVALSPLGALV |
| | | ACYKGVSCSIGSNQVGIIKQLPKGCSYITNQDADT |
| | | VTIDNTVYQLSKVEGEQHVIKGRPVSSSFDPIRFP |
| | | EDQFNVPLDQVFESIENSQALVDQSNKILNSAEKG |
| | | NT |
| | | F2 (SEQ ID NO: 161): |
| | | LKESYLEESCSTITEGYLSVLRTGWYTNVFTLEVG |
| | | DVENLTCTDGPSLIKTELDLTKSALRELKTVSADQ |
| | | LAREEQIENPRQSR |

TABLE 24
Exemplary hMPV B F Protein Mutants Comprising
Engineered Disulfide Mutations and Glycine
Replacement Mutations

| | | Amino Acid Sequence (residues |
|---|---|---|
| | | 103-489 for F1 polypeptide and |
| Mutant | Muta- | residues 19-102 for F2 |
| ID | tions | polypeptide) |
| hMPV | A140C- | F1 (SEQ ID NO: 162): |
| 123 | S149C, | FVLGAIALGVATAAAVTAGIAIAKTIRLESEVNAI |
| | G239A, | KGCLKTTNEAVCTLGNGVRVLATAVRELKEFVSKN |
| | T411C- | LTSAINKNKCDIADLKMAVSFSQFNRRFLNVVRQF |
| | Q434C | SDNAGITPAISLDLMNDAELARAVSYMPTSAAQIK |
| | | LMLENRAMVRRKGFGILIGVYGSSVIYMVQLPIFG |
| | | VINTPCWIIKAAPSCSEKDGNYACLLREDQGWYCK |
| | | NAGSTVYYPNEKDCETRGDHVFCDTAAGINVAEQS |
| | | RECNINISTTNYPCKVSTGRHPISMVALSPLGALV |
| | | ACYKGVSCSIGSNQVGIIKQLPKGCSYICNQDADT |
| | | VTIDNTVYQLSKVEGECHVIKGRPVSSSFDPIRFP |
| | | EDQFNVALDQVFESIENSQALVDQSNKILNSAEKG |
| | | NT |
| | | F2 (SEQ ID NO: 163): |
| | | LKESYLEESCSTITEGYLSVLRTGWYTNVFTLEVG |
| | | DVENLTCTDGPSLIKTELDLTKSALRELKTVSADQ |
| | | LAREEQIENPRQSR |
| hMPV | L141C- | F1 (SEQ ID NO: 164): |
| 125 | A161C, | FVLGAIALGVATAAAVTAGIAIAKTIRLESEVNAI |
| | G239A, | KGACKTTNEAVSTLGNGVRVLATCVRELKEFVSKN |
| | T411C- | LTSAINKNKCDIADLKMAVSFSQFNRRFLNVVRQF |
| | Q434C | SDNAGITPAISLDLMNDAELARAVSYMPTSAAQIK |
| | | LMLENRAMVRRKGFGILIGVYGSSVIYMVQLPIFG |
| | | VINTPCWIIKAAPSCSEKDGNYACLLREDQGWYCK |
| | | NAGSTVYYPNEKDCETRGDHVFCDTAAGINVAEQS |
| | | RECNINISTTNYPCKVSTGRHPISMVALSPLGALV |
| | | ACYKGVSCSIGSNQVGIIKQLPKGCSYICNQDADT |
| | | VTIDNTVYQLSKVEGECHVIKGRPVSSSFDPIRFP |
| | | EDQFNVALDQVFESIENSQALVDQSNKILNSAEKG |
| | | NT |
| | | F2 (SEQ ID NO: 165): |
| | | LKESYLEESCSTITEGYLSVLRTGWYTNVFTLEVG |
| | | DVENLTCTDGPSLIKTELDLTKSALRELKTVSADQ |
| | | LAREEQIENPRQSR |

TABLE 25
Exemplary hMPV B F Protein Mutants Comprising Engineered
Disulfide Mutations and Cavity Filling Mutations

| | | Amino Acid Sequence (residues 103-489 |
|---|---|---|
| Mutant | | for F1 polypeptide and residues 19-102 |
| ID | Mutations | for F2 polypeptide) |
| hMPV118 | T49I, T150C- | F1 (SEQ ID NO: 166): |
| | R156C | FVLGAIALGVATAAAVTAGIAIAKTIRLESEVNAIKGALKTTN |
| | | EAVSCLGNGVCVLATAVRELKEFVSKNLTSAINKNKCDIA |
| | | DLKMAVSFSQFNRRFLNVVRQFSDNAGITPAISLDLMNDA |
| | | ELARAVSYMPTSAGQIKLMLENRAMVRRKGFGILIGVYGS |
| | | SVIYMVQLPIFGVINTPCWIIKAAPSCSEKDGNYACLLRED |
| | | QGWYCKNAGSTVYYPNEKDCETRGDHVFCDTAAGINVA |
| | | EQSRECNINISTTNYPCKVSTGRHPISMVALSPLGALVACY |
| | | KGVSCSIGSNQVGIIKQLPKGCSYITNQDADTVTIDNTVYQ |
| | | LSKVEGEQHVIKGRPVSSSFDPIRFPEDQFNVALDQVFESI |
| | | ENSQALVDQSNKILNSAEKGNT |
| | | F2 (SEQ ID NO: 167): |
| | | LKESYLEESCSTITEGYLSVLRTGWYTNVFILEVGDVENLT |
| | | CTDGPSLIKTELDLTKSALRELKTVSADQLAREEQIENPRQ |
| | | SR |
| hMPV137 | T49I, A140C- | F1 (SEQ ID NO: 168): |
| | S149C | FVLGAIALGVATAAAVTAGIAIAKTIRLESEVNAIKGCLKTTN |
| | | EAVCTLGNGVRVLATAVRELKEFVSKNLTSAINKNKCDIAD |
| | | LKMAVSFSQFNRRFLNVVRQFSDNAGITPAISLDLMNDAE |
| | | LARAVSYMPTSAGQIKLMLENRAMVRRKGFGILIGVYGSS |
| | | VIYMVQLPIFGVINTPCWIIKAAPSCSEKDGNYACLLREDQ |
| | | GWYCKNAGSTVYYPNEKDCETRGDHVFCDTAAGINVAE |
| | | QSRECNINISTTNYPCKVSTGRHPISMVALSPLGALVACYK |
| | | GVSCSIGSNQVGIIKQLPKGCSYITNQDADTVTIDNTVYQL |
| | | SKVEGEQHVIKGRPVSSSFDPIRFPEDQFNVALDQVFESI |
| | | ENSQALVDQSNKILNSAEKGNT |
| | | F2 (SEQ ID NO: 169): |
| | | LKESYLEESCSTITEGYLSVLRTGWYTNVFILEVGDVENLT |
| | | CTDGPSLIKTELDLTKSALRELKTVSADQLAREEQIENPRQ |
| | | SR |
| hMPV139 | T49I, L141C- | F1 (SEQ ID NO: 170): |
| | A161C, | FVLGAIALGVATAAAVTAGIAIAKTIRLESEVNAIKGACKTT |
| | T411C-Q434C | NEAVSTLGNGVRVLATCVRELKEFVSKNLTSAINKNKCDIA |
| | | DLKMAVSFSQFNRRFLNVVRQFSDNAGITPAISLDLMNDA |
| | | ELARAVSYMPTSAGQIKLMLENRAMVRRKGFGILIGVYGS |
| | | SVIYMVQLPIFGVINTPCWIIKAAPSCSEKDGNYACLLRED |
| | | QGWYCKNAGSTVYYPNEKDCETRGDHVFCDTAAGINVA |
| | | EQSRECNINISTTNYPCKVSTGRHPISMVALSPLGALVACY |
| | | KGVSCSIGSNQVGIIKQLPKGCSYICNQDADTVTIDNTVYQ |
| | | LSKVEGECHVIKGRPVSSSFDPIRFPEDQFNVALDQVFESI |
| | | ENSQALVDQSNKILNSAEKGNT |
| | | F2 (SEQ ID NO: 171): |
| | | LKESYLEESCSTITEGYLSVLRTGWYTNVFILEVGDVENLT |
| | | CTDGPSLIKTELDLTKSALRELKTVSADQLAREEQIENPRQ |
| | | SR |
| hMPV141 | L141C-A161C, | F1 (SEQ ID NO: 172): |
| | S149T | FVLGAIALGVATAAAVTAGIAIAKTIRLESEVNAIKGACKTT |
| | | NEAVTTLGNGVRVLATCVRELKEFVSKNLTSAINKNKCDIA |
| | | DLKMAVSFSQFNRRFLNVVRQFSDNAGITPAISLDLMNDA |
| | | ELARAVSYMPTSAGQIKLMLENRAMVRRKGFGILIGVYGS |
| | | SVIYMVQLPIFGVINTPCWIIKAAPSCSEKDGNYACLLRED |
| | | QGWYCKNAGSTVYYPNEKDCETRGDHVFCDTAAGINVA |
| | | EQSRECNINISTTNYPCKVSTGRHPISMVALSPLGALVACY |
| | | KGVSCSIGSNQVGIIKQLPKGCSYITNQDADTVTIDNTVYQ |
| | | LSKVEGEQHVIKGRPVSSSFDPIRFPEDQFNVALDQVFESI |
| | | ENSQALVDQSNKILNSAEKGNT |
| | | F2 (SEQ ID NO: 173): |
| | | LKESYLEESCSTITEGYLSVLRTGWYTNVFTLEVGDVENL |
| | | TCTDGPSLIKTELDLTKSALRELKTVSADQLAREEQIENPR |
| | | QSR |
| hMPV143 | T49I, L141C- | F1 (SEQ ID NO: 174): |
| | A161C, S149T | FVLGAIALGVATAAAVTAGIAIAKTIRLESEVNAIKGACKTT |
| | | NEAVTTLGNGVRVLATCVRELKEFVSKNLTSAINKNKCDIA |
| | | DLKMAVSFSQFNRRFLNVVRQFSDNAGITPAISLDLMNDA |
| | | ELARAVSYMPTSAGQIKLMLENRAMVRRKGFGILIGVYGS |
| | | SVIYMVQLPIFGVINTPCWIIKAAPSCSEKDGNYACLLRED |
| | | QGWYCKNAGSTVYYPNEKDCETRGDHVFCDTAAGINVA |
| | | EQSRECNINISTTNYPCKVSTGRHPISMVALSPLGALVACY |
| | | KGVSCSIGSNQVGIIKQLPKGCSYITNQDADTVTIDNTVYQ |
| | | LSKVEGEQHVIKGRPVSSSFDPIRFPEDQFNVALDQVFESI |
| | | ENSQALVDQSNKILNSAEKGNT |
| | | F2 (SEQ ID NO: 175): |
| | | LKESYLEESCSTITEGYLSVLRTGWYTNVFILEVGDVENLT |
| | | CTDGPSLIKTELDLTKSALRELKTVSADQLAREEQIENPRQ |
| | | SR |

TABLE 26
Exemplary hMPV B F Protein Mutants Comprising
Engineered Disulfide Mutations, Proline
Substitution Mutations, Glycine Replacement
Mutations, and Cavity Filling Mutations

| | | Amino Acid Sequence (residues 103-489 |
|---|---|---|
| Mutant | | for F1 polypeptide and residues 19-102 |
| ID | Mutations | for F2 polypeptide) |
| hMPV126 | T49I, T150C- | F1 (SEQ ID NO: 176): |
| | R156C, | FVLGAIALGVATAAAVTAGIAIAKTIRLESEVNAIKGALKTTN |
| | G239A, A459P | EAVSCLGNGVCVLATAVRELKEFVSKNLTSAINKNKCDIA |
| | | DLKMAVSFSQFNRRFLNVVRQFSDNAGITPAISLDLMNDA |
| | | ELARAVSYMPTSAAQIKLMLENRAMVRRKGFGILIGVYGS |
| | | SVIYMVQLPIFGVINTPCWIIKAAPSCSEKDGNYACLLRED |
| | | QGWYCKNAGSTVYYPNEKDCETRGDHVFCDTAAGINVA |
| | | EQSRECNINISTTNYPCKVSTGRHPISMVALSPLGALVACY |
| | | KGVSCSIGSNQVGIIKQLPKGCSYITNQDADTVTIDNTVYQ |
| | | LSKVEGEQHVIKGRPVSSSFDPIRFPEDQFNVPLDQVFESI |
| | | ENSQALVDQSNKILNSAEKGNT |
| | | F2 (SEQ ID NO: 177): |
| | | LKESYLEESCSTITEGYLSVLRTGWYTNVFILEVGDVENLT |
| | | CTDGPSLIKTELDLTKSALRELKTVSADQLAREEQIENPRQ |
| | | SR |
| hMPV128 | T49I, A140C- | F1 (SEQ ID NO: 178): |
| | S149C, | FVLGAIALGVATAAAVTAGIAIAKTIRLESEVNAIKGCLKTTN |
| | G239A, | EAVCTLGNGVRVLATAVRELKEFVSKNLTSAINKNKCDIAD |
| | T411C-Q434C, | LKMAVSFSQFNRRFLNVVRQFSDNAGITPAISLDLMNDAE |
| | A459P | LARAVSYMPTSAAQIKLMLENRAMVRRKGFGILIGVYGSS |
| | | VIYMVQLPIFGVINTPCWIIKAAPSCSEKDGNYACLLREDQ |
| | | GWYCKNAGSTVYYPNEKDCETRGDHVFCDTAAGINVAE |
| | | QSRECNINISTTNYPCKVSTGRHPISMVALSPLGALVACYK |
| | | GVSCSIGSNQVGIIKQLPKGCSYICNQDADTVTIDNTVYQL |
| | | SKVEGECHVIKGRPVSSSFDPIRFPEDQFNVPLDQVFESI |
| | | ENSQALVDQSNKILNSAEKGNT |
| | | F2 (SEQ ID NO: 179): |
| | | LKESYLEESCSTITEGYLSVLRTGWYTNVFILEVGDVENLT |
| | | CTDGPSLIKTELDLTKSALRELKTVSADQLAREEQIENPRQ |
| | | SR |
| hMPV129 | T49I, A140C- | F1 (SEQ ID NO: 180): |
| | S149C, | FVLGAIALGVATAAAVTAGIAIAKTIRLESEVNAIKGCLKTTN |
| | G239A, T365I, | EAVCTLGNGVRVLATAVRELKEFVSKNLTSAINKNKCDIAD |
| | T411C-Q434C, | LKMAVSFSQFNRRFLNVVRQFSDNAGITPAISLDLMNDAE |
| | A459P | LARAVSYMPTSAAQIKLMLENRAMVRRKGFGILIGVYGSS |
| | | VIYMVQLPIFGVINTPCWIIKAAPSCSEKDGNYACLLREDQ |
| | | GWYCKNAGSTVYYPNEKDCETRGDHVFCDTAAGINVAE |
| | | QSRECNINISTTNYPCKVSIGRHPISMVALSPLGALVACYK |
| | | GVSCSIGSNQVGIIKQLPKGCSYICNQDADTVTIDNTVYQL |
| | | SKVEGECHVIKGRPVSSSFDPIRFPEDQFNVPLDQVFESI |
| | | ENSQALVDQSNKILNSAEKGNT |
| | | F2 (SEQ ID NO: 181): |
| | | LKESYLEESCSTITEGYLSVLRTGWYTNVFILEVGDVENLT |
| | | CTDGPSLIKTELDLTKSALRELKTVSADQLAREEQIENPRQ |
| | | SR |
| hMPV131 | T49I, L141C- | F1 (SEQ ID NO: 182): |
| | A161C, | FVLGAIALGVATAAAVTAGIAIAKTIRLESEVNAIKGACKTT |
| | G239A, | NEAVSTLGNGVRVLATCVRELKEFVSKNLTSAINKNKCDIA |
| | T411C-Q434C, | DLKMAVSFSQFNRRFLNVVRQFSDNAGITPAISLDLMNDA |
| | A459P | ELARAVSYMPTSAAQIKLMLENRAMVRRKGFGILIGVYGS |
| | | SVIYMVQLPIFGVINTPCWIIKAAPSCSEKDGNYACLLRED |
| | | QGWYCKNAGSTVYYPNEKDCETRGDHVFCDTAAGINVA |
| | | EQSRECNINISTTNYPCKVSTGRHPISMVALSPLGALVACY |
| | | KGVSCSIGSNQVGIIKQLPKGCSYICNQDADTVTIDNTVYQ |
| | | LSKVEGECHVIKGRPVSSSFDPIRFPEDQFNVPLDQVFESI |
| | | ENSQALVDQSNKILNSAEKGNT |
| | | F2 (SEQ ID NO: 183): |
| | | LKESYLEESCSTITEGYLSVLRTGWYTNVFILEVGDVENLT |
| | | CTDGPSLIKTELDLTKSALRELKTVSADQLAREEQIENPRQ |
| | | SR |
| hMPV132 | L141C-A161C, | F1 (SEQ ID NO: 184): |
| | S149T, | FVLGAIALGVATAAAVTAGIAIAKTIRLESEVNAIKGACKTT |
| | G239A, | NEAVTTLGNGVRVLATCVRELKEFVSKNLTSAINKNKCDIA |
| | T411C-Q434C, | DLKMAVSFSQFNRRFLNVVRQFSDNAGITPAISLDLMNDA |
| | A459P | ELARAVSYMPTSAAQIKLMLENRAMVRRKGFGILIGVYGS |
| | | SVIYMVQLPIFGVINTPCWIIKAAPSCSEKDGNYACLLRED |
| | | QGWYCKNAGSTVYYPNEKDCETRGDHVFCDTAAGINVA |
| | | EQSRECNINISTTNYPCKVSTGRHPISMVALSPLGALVACY |
| | | KGVSCSIGSNQVGIIKQLPKGCSYICNQDADTVTIDNTVYQ |
| | | LSKVEGECHVIKGRPVSSSFDPIRFPEDQFNVPLDQVFESI |
| | | ENSQALVDQSNKILNSAEKGNT |
| | | F2 (SEQ ID NO: 185): |
| | | LKESYLEESCSTITEGYLSVLRTGWYTNVFTLEVGDVENL |
| | | TCTDGPSLIKTELDLTKSALRELKTVSADQLAREEQIENPR |
| | | QSR |
| hMPV133 | T49I, L141C- | F1 (SEQ ID NO: 186): |
| | A161C, | FVLGAIALGVATAAAVTAGIAIAKTIRLESEVNAIKGACKTT |
| | S149T, | NEAVTTLGNGVRVLATCVRELKEFVSKNLTSAINKNKCDIA |
| | G239A, | DLKMAVSFSQFNRRFLNVVRQFSDNAGITPAISLDLMNDA |
| | T411C-Q434C, | ELARAVSYMPTSAAQIKLMLENRAMVRRKGFGILIGVYGS |
| | A459P | SVIYMVQLPIFGVINTPCWIIKAAPSCSEKDGNYACLLRED |
| | | QGWYCKNAGSTVYYPNEKDCETRGDHVFCDTAAGINVA |
| | | EQSRECNINISTTNYPCKVSTGRHPISMVALSPLGALVACY |
| | | KGVSCSIGSNQVGIIKQLPKGCSYICNQDADTVTIDNTVYQ |
| | | LSKVEGECHVIKGRPVSSSFDPIRFPEDQFNVPLDQVFESI |
| | | ENSQALVDQSNKILNSAEKGNT |
| | | F2 (SEQ ID NO: 187): |
| | | LKESYLEESCSTITEGYLSVLRTGWYTNVFILEVGDVENLT |
| | | CTDGPSLIKTELDLTKSALRELKTVSADQLAREEQIENPRQ |
| | | SR |
| hMPV134 | T49I, L141C- | F1 (SEQ ID NO: 188): |
| | A161C, | FVLGAIALGVATAAAVTAGIAIAKTIRLESEVNAIKGACKTT |
| | S149T, | NEAVTTLGNGVRVLATCVRELKEFVSKNLTSAINKNKCDIA |
| | G239A, T3651, | DLKMAVSFSQFNRRFLNVVRQFSDNAGITPAISLDLMNDA |
| | T411C-Q434C, | ELARAVSYMPTSAAQIKLMLENRAMVRRKGFGILIGVYGS |
| | A459P | SVIYMVQLPIFGVINTPCWIIKAAPSCSEKDGNYACLLRED |
| | | QGWYCKNAGSTVYYPNEKDCETRGDHVFCDTAAGINVA |
| | | EQSRECNINISTTNYPCKVSIGRHPISMVALSPLGALVACY |
| | | KGVSCSIGSNQVGIIKQLPKGCSYICNQDADTVTIDNTVYQ |
| | | LSKVEGECHVIKGRPVSSSFDPIRFPEDQFNVPLDQVFESI |
| | | ENSQALVDQSNKILNSAEKGNT |
| | | F2 (SEQ ID NO: 189): |
| | | LKESYLEESCSTITEGYLSVLRTGWYTNVFILEVGDVENLT |
| | | CTDGPSLIKTELDLTKSALRELKTVSADQLAREEQIENPRQ |
| | | SR |

TABLE 27
Exemplary hMPV B F Protein Mutants Comprising
Engineered Disulfide Mutations, Proline
Substitution Mutations and Cavity Filling
Mutations

| | | Amino Acid Sequence (residues |
|---|---|---|
| | | 103-489 for F1 polypeptide |
| Mutant | Muta- | and residues 19-102 for F2 |
| ID | tions | polypeptide) |
| hMPV | T49I, | F1 (SEQ ID NO: 190): |
| 119 | T150C- | FVLGAIALGVATAAAVTAGIAIAKTIRLESEVNAI |
| | R156C, | KGALKTTNEAVSCLGNGVCVLATAVRELKEFVSKN |
| | A459P | LTSAINKNKCDIADLKMAVSFSQFNRRFLNVVRQF |
| | | SDNAGITPAISLDLMNDAELARAVSYMPTSAGQIK |
| | | LMLENRAMVRRKGFGILIGVYGSSVIYMVQLPIFG |
| | | VINTPCWIIKAAPSCSEKDGNYACLLREDQGWYCK |
| | | NAGSTVYYPNEKDCETRGDHVFCDTAAGINVAEQS |
| | | RECNINISTTNYPCKVSTGRHPISMVALSPLGALV |
| | | ACYKGVSCSIGSNQVGIIKQLPKGCSYITNQDADT |
| | | VTIDNTVYQLSKVEGEQHVIKGRPVSSSFDPIRFP |
| | | EDQFNVPLDQVFESIENSQALVDQSNKILNSAEKG |
| | | NT |
| | | F2 (SEQ ID NO: 191): |
| | | LKESYLEESCSTITEGYLSVLRTGWYTNVFILEVG |
| | | DVENLTCTDGPSLIKTELDLTKSALRELKTVSADQ |
| | | LAREEQIENPRQSR |
| hMPV | T49I, | F1 (SEQ ID NO: 192): |
| 138 | A140C- | FVLGAIALGVATAAAVTAGIAIAKTIRLESEVNAI |
| | S149C, | KGCLKTTNEAVCTLGNGVRVLATAVRELKEFVSKN |
| | A459P | LTSAINKNKCDIADLKMAVSFSQFNRRFLNVVRQF |
| | | SDNAGITPAISLDLMNDAELARAVSYMPTSAGQIK |
| | | LMLENRAMVRRKGFGILIGVYGSSVIYMVQLPIFG |
| | | VINTPCWIIKAAPSCSEKDGNYACLLREDQGWYCK |
| | | NAGSTVYYPNEKDCETRGDHVFCDTAAGINVAEQS |
| | | RECNINISTTNYPCKVSTGRHPISMVALSPLGALV |
| | | ACYKGVSCSIGSNQVGIIKQLPKGCSYITNQDADT |
| | | VTIDNTVYQLSKVEGEQHVIKGRPVSSSFDPIRFP |
| | | EDQFNVPLDQVFESIENSQALVDQSNKILNSAEKG |
| | | NT |
| | | F2 (SEQ ID NO: 193): |
| | | LKESYLEESCSTITEGYLSVLRTGWYTNVFILEVG |
| | | DVENLTCTDGPSLIKTELDLTKSALRELKTVSADQ |
| | | LAREEQIENPRQSR |
| hMPV | T49I, | F1 (SEQ ID NO: 194): |
| 140 | L141C- | FVLGAIALGVATAAAVTAGIAIAKTIRLESEVNAI |
| | A161C, | KGACKTTNEAVSTLGNGVRVLATCVRELKEFVSKN |
| | T411C- | LTSAINKNKCDIADLKMAVSFSQFNRRFLNVVRQF |
| | Q434C, | SDNAGITPAISLDLMNDAELARAVSYMPTSAGQIK |
| | A459P | LMLENRAMVRRKGFGILIGVYGSSVIYMVQLPIFG |
| | | VINTPCWIIKAAPSCSEKDGNYACLLREDQGWYCK |
| | | NAGSTVYYPNEKDCETRGDHVFCDTAAGINVAEQS |
| | | RECNINISTTNYPCKVSTGRHPISMVALSPLGALV |
| | | ACYKGVSCSIGSNQVGIIKQLPKGCSYICNQDADT |
| | | VTIDNTVYQLSKVEGECHVIKGRPVSSSFDPIRFP |
| | | EDQFNVPLDQVFESIENSQALVDQSNKILNSAEKG |
| | | NT |
| | | F2 (SEQ ID NO: 195): |
| | | LKESYLEESCSTITEGYLSVLRTGWYTNVFILEVG |
| | | DVENLTCTDGPSLIKTELDLTKSALRELKTVSADQ |
| | | LAREEQIENPRQSR |
| hMPV | L141C- | F1 (SEQ ID NO: 196): |
| 142 | A161C, | FVLGAIALGVATAAAVTAGIAIAKTIRLESEVNAI |
| | S149T, | KGACKTTNEAVTTLGNGVRVLATCVRELKEFVSKN |
| | A459P | LTSAINKNKCDIADLKMAVSFSQFNRRFLNVVRQF |
| | | SDNAGITPAISLDLMNDAELARAVSYMPTSAGQIK |
| | | LMLENRAMVRRKGFGILIGVYGSSVIYMVQLPIFG |
| | | VINTPCWIIKAAPSCSEKDGNYACLLREDQGWYCK |
| | | NAGSTVYYPNEKDCETRGDHVFCDTAAGINVAEQS |
| | | RECNINISTTNYPCKVSTGRHPISMVALSPLGALV |
| | | ACYKGVSCSIGSNQVGIIKQLPKGCSYITNQDADT |
| | | VTIDNTVYQLSKVEGEQHVIKGRPVSSSFDPIRFP |
| | | EDQFNVPLDQVFESIENSQALVDQSNKILNSAEKG |
| | | NT |
| | | F2 (SEQ ID NO: 197): |
| | | LKESYLEESCSTITEGYLSVLRTGWYTNVFTLEVG |
| | | DVENLTCTDGPSLIKTELDLTKSALRELKTVSADQ |
| | | LAREEQIENPRQSR |
| hMPV | T49I, | F1 (SEQ ID NO: 198): |
| 144 | L141C- | FVLGAIALGVATAAAVTAGIAIAKTIRLESEVNAI |
| | A161C, | KGACKTTNEAVTTLGNGVRVLATCVRELKEFVSKN |
| | S149T, | LTSAINKNKCDIADLKMAVSFSQFNRRFLNVVRQF |
| | A459P | SDNAGITPAISLDLMNDAELARAVSYMPTSAGQIK |
| | | LMLENRAMVRRKGFGILIGVYGSSVIYMVQLPIFG |
| | | VINTPCWIIKAAPSCSEKDGNYACLLREDQGWYCK |
| | | NAGSTVYYPNEKDCETRGDHVFCDTAAGINVAEQS |
| | | RECNINISTTNYPCKVSTGRHPISMVALSPLGALV |
| | | ACYKGVSCSIGSNQVGIIKQLPKGCSYITNQDADT |
| | | VTIDNTVYQLSKVEGEQHVIKGRPVSSSFDPIRFP |
| | | EDQFNVPLDQVFESIENSQALVDQSNKILNSAEKG |
| | | NT |
| | | F2 (SEQ ID NO: 199): |
| | | LKESYLEESCSTITEGYLSVLRTGWYTNVFILEVG |
| | | DVENLTCTDGPSLIKTELDLTKSALRELKTVSADQ |
| | | LAREEQIENPRQSR |

TABLE 28
Exemplary hMPV B F Protein Mutants Comprising
Engineered Disulfide Mutations, Proline
Substitution Mutations and Glycine Replacement
Mutations

| | | Amino Acid Sequence (residues |
|---|---|---|
| | | 103-489 for F1 polypeptide and |
| Mutant | Muta- | residues 19-102 for F2 |
| ID | tions | polypeptide) |
| hMPV | A140C- | F1 (SEQ ID NO: 200): |
| 127 | S149C, | FVLGAIALGVATAAAVTAGIAIAKTIRLESEVNAI |
| | G239A, | KGCLKTTNEAVCTLGNGVRVLATAVRELKEFVSKN |
| | T411C- | LTSAINKNKCDIADLKMAVSFSQFNRRFLNVVRQF |
| | Q434C, | SDNAGITPAISLDLMNDAELARAVSYMPTSAAQIK |
| | A459P | LMLENRAMVRRKGFGILIGVYGSSVIYMVQLPIFG |
| | | VINTPCWIIKAAPSCSEKDGNYACLLREDQGWYCK |
| | | NAGSTVYYPNEKDCETRGDHVFCDTAAGINVAEQS |
| | | RECNINISTTNYPCKVSTGRHPISMVALSPLGALV |
| | | ACYKGVSCSIGSNQVGIIKQLPKGCSYICNQDADT |
| | | VTIDNTVYQLSKVEGECHVIKGRPVSSSFDPIRFP |
| | | EDQFNVPLDQVFESIENSQALVDQSNKILNSAEKG |
| | | NT |
| | | F2 (SEQ ID NO: 201): |
| | | LKESYLEESCSTITEGYLSVLRTGWYTNVFTLEVG |
| | | DVENLTCTDGPSLIKTELDLTKSALRELKTVSADQ |
| | | LAREEQIENPRQSR |
| hMPV | L141C- | F1 (SEQ ID NO: 202): |
| 130 | A161C, | FVLGAIALGVATAAAVTAGIAIAKTIRLESEVNAI |
| | G239A, | KGACKTTNEAVSTLGNGVRVLATCVRELKEFVSKN |
| | T411C- | LTSAINKNKCDIADLKMAVSFSQFNRRFLNVVRQF |
| | Q434C, | SDNAGITPAISLDLMNDAELARAVSYMPTSAAQIK |
| | A459P | LMLENRAMVRRKGFGILIGVYGSSVIYMVQLPIFG |
| | | VINTPCWIIKAAPSCSEKDGNYACLLREDQGWYCK |
| | | NAGSTVYYPNEKDCETRGDHVFCDTAAGINVAEQS |
| | | RECNINISTTNYPCKVSTGRHPISMVALSPLGALV |
| | | ACYKGVSCSIGSNQVGIIKQLPKGCSYICNQDADT |
| | | VTIDNTVYQLSKVEGECHVIKGRPVSSSFDPIRFP |
| | | EDQFNVPLDQVFESIENSQALVDQSNKILNSAEKG |
| | | NT |
| | | F2 (SEQ ID NO: 203): |
| | | LKESYLEESCSTITEGYLSVLRTGWYTNVFTLEVG |
| | | DVENLTCTDGPSLIKTELDLTKSALRELKTVSADQ |
| | | LAREEQIENPRQSR |

TABLE 29
Exemplary hMPV B F Protein Mutants Comprising
Proline Substitution Mutations and Cavity
Filling Mutations

| | | Amino Acid Sequence (residues |
|---|---|---|
| | | 103-489 for F1 polypeptide and |
| Mutant | Muta- | residues 19-102 for F2 |
| ID | tions | polypeptide) |
| hMPV | T49I, | F1 (SEQ ID NO: 204): |
| 135 | S149T, | FVLGAIALGVATAAAVTAGIAIAKTIRLESEVNAI |
| | A459P | KGALKTTNEAVTTLGNGVRVLATAVRELKEFVSKN |
| | | LTSAINKNKCDIADLKMAVSFSQFNRRFLNVVRQF |
| | | SDNAGITPAISLDLMNDAELARAVSYMPTSAGQIK |
| | | LMLENRAMVRRKGFGILIGVYGSSVIYMVQLPIFG |
| | | VINTPCWIIKAAPSCSEKDGNYACLLREDQGWYCK |
| | | NAGSTVYYPNEKDCETRGDHVFCDTAAGINVAEQS |
| | | RECNINISTTNYPCKVSTGRHPISMVALSPLGALV |
| | | ACYKGVSCSIGSNQVGIIKQLPKGCSYITNQDADT |
| | | VTIDNTVYQLSKVEGEQHVIKGRPVSSSFDPIRFP |
| | | EDQFNVPLDQVFESIENSQALVDQSNKILNSAEKG |
| | | NT |
| | | F2 (SEQ ID NO: 205): |
| | | LKESYLEESCSTITEGYLSVLRTGWYTNVFILEVG |
| | | DVENLTCTDGPSLIKTELDLTKSALRELKTVSADQ |
| | | LAREEQIENPRQSR |

### Example 6. hMPV B F Protein Mutant Expression Vector Construction

A nucleic acid molecule encoding the consensus truncated hMPV B F0 polypeptide set forth in SEQ ID NO: 129 was mutated using standard molecular biology techniques to encode a precursor polypeptide for a hMPV F mutant having the introduced amino acid mutations disclosed in example 5. The structure and components of the precursor polypeptide are set forth in FIG. 1 and SEQ ID NO: 129. The precursor polypeptide comprises of a signal peptide (residues 1-18), F2 polypeptide (residues 19-102), F1 polypeptide (residues 103-489), T4 fibritin foldon (residues 494-520), PreScission cleavage site (residues 524-531), Strep Tag II (residues 535-542), and linker sequences (residues 490-493, 521-523, 532-534, 543-546).

The protein sequence of SEQ ID NO: 129 was submitted for mammalian codon optimization by Genscript (Piscataway, NJ). The nucleotide sequence was introduced into a commercially available expression vector, pcDNA3.1/Zeo(+) (ThermoFisher Scientific, Waltham, MA) that has been modified to encode the CAG promoter (Yamamura et al., Gene, 108(2), 193-199, 1991) in place of the CMV promoter. Double stranded DNA fragments were purchased from Integrated DNA Technologies (Coralville, IA). DNA fragments of the mutagenized F allele not synthesized were generated and amplified by polymerase chain reaction (PCR) with Phusion Flash High-Fidelity PCR Master Mix (ThermoFisher Scientific). Following purification of the linearized expression vector digested using BamHI and Notl, gene fragments of the mutagenized F allele were inserted into the expression vector with NEBuilder HiFi DNA Assembly Kit (New England Biolabs, Ipswich, MA). The presence of the intended sequence was confirmed by Sanger DNA sequencing. Plasmid DNA for transfection into ExpiCHO cells was purified with the Qiagen Plasmid Plus Midi Kit (Qiagen, Valencia, CA). For all commercial kits or reagents, procedures were performed according to the manufacturer's protocol.

### Example 7. Transfection OF hMPV B F Protein Mutants

Proteins for hMPV B F mutant evaluation were produced by transient transfection of ExpiCHO cells (ThermoFisher Scientific) with DNA plasmids assembled and prepared as described in Example 6. Transient transfections were carried out according to the manufacturer's protocol. On day 5 post transfection, the cultures were centrifuged, and supernatants were separated from cell pellets. The crude cell supernatants were used for in vitro assays described herein.

### Example 8. Expression and Conformational Integrity OF hMPV B F Protein Mutants

The OCTET HTX (Sartorius, Gottingen, Germany) instrument was used to evaluate the expression and conformational integrity for each mutant. All measurements were conducted at 30° C. temperature in 96-well black plates (Corning, Corning, NY) with a final volume of 240 μL per well at a constant agitation rate of 1000 RPM.
8A: Quantitation of Expression of hMPV B F Protein Mutants

Crude cell supernatant was used to quantitate the expression levels of hMPV B F protein mutants. Anti-Murine IgG Quantitation (AMQ) Biosensors (Sartorius) were first equilibrated in PB before being dipped into more PB to establish the initial baseline. Biosensors were incubated with a mouse Strep-Tag® II monoclonal antibody (mAb) (Novagen, EMD Millipore Corporation, Temecula, CA) before being equilibrated in PB to establish the experimental baseline. The mAb bound biosensors were then dipped into crude cell culture supernatant for 2.5 minutes. OCTET data analysis software (version 12.0 and 12.2, Sartorius) was used to generate a standard curve from a serially diluted purified protein reference within the same assay. Titers for protein mutants were then determined based on the standard curve.

The results are presented in Table 30 and 30.1. The highest expressing amino acid substitutions identified from the screens of the individual engineered disulfide mutants, proline mutations, glycine replacement mutations and cavity filling mutants were combined into the combination mutants. The protein expression of combination mutants was measured using the same procedure as described. The results are presented in Table 31.

TABLE 30
Protein expression and thermal stability
of hMPV B F protein mutants.

| | Protein | Response Shift with hMPV-2 mAb | | |
|---|---|---|---|---|
| | expression | No stress | 53° C. stress | 56° C. stress |
| Mutant ID | (mg/L) | (nm) | resistance (nm) | resistance (nm) |
| WT Control | 3.1 | 0.867 | 0.418 | 0.005 |
| hMPV100 | 2.0 | 0.863 | 0.854 | 0.779 |
| hMPV101 | 9.6 | 0.707 | 0.716 | 0.649 |
| hMPV102 | 12.9 | 0.840 | 0.572 | 0.007 |
| hMPV103 | 9.9 | 0.814 | 0.588 | 0.041 |
| hMPV104 | 8.2 | 0.850 | 0.336 | −0.110 |
| hMPV105 | 2.1 | 0.906 | 0.785 | 0.064 |
| hMPV106 | 3.3 | 0.912 | 0.828 | 0.397 |
| hMPV107 | 2.4 | 0.890 | 0.846 | 0.519 |
| hMPV108 | 1.8 | 0.890 | 0.802 | 0.040 |

TABLE 30.1
Protein expression and thermal stability of hMPV B F protein
mutants with additional thermal stress conditions.

| | Protein | Response Shift with hMPV-2 mAb | | |
|---|---|---|---|---|
| | expression | No stress | 56° C. stress | 63° C. stress |
| Mutant ID | (mg/L) | (nm) | resistance (nm) | resistance (nm) |
| WT Control | 3.7 | 0.820 | 0.122 | 0.017 |
| hMPV116 | 11.6 | 0.855 | 0.792 | −0.010 |

8B: Conformational Integrity of hMPV B F Protein Mutants

Conformation integrity of hMPV B F protein mutants were evaluated by a thermal stress experiment. Crude cell culture supernatants were normalized based on the results of the titer quantitation and then incubated for 1 hour at room temperature and two higher temperatures. For the testing of hMPV B single mutants, the two temperatures were 53° C. and 56° C. Some additional mutants were stressed at higher temperature, 56° C., and 63° C., as shown in Table 31. After the incubation, the protein mutants were probed with prefusion specific monoclonal antibody hMPV-2 by OCTET HTX. Anti-Human IgG Fc Capture (AHC) biosensors were first equilibrated in PB before being dipped into more PB for to establish the initial baseline. Biosensors were incubated with hMPV-2 before being equilibrated in PB to establish the experimental baseline. The mAb-bound biosensors were then dipped into the thermal stresses cell culture supernatants for 5 minutes. The kinetics analysis was done by OCTET data analysis software (version 12.2, Sartorius) based on curve fitting of the entire associate step.

The results are presented in Table 30 and Table 30.1. The most stabilizing amino acid substitutions identified from the screens of the individual engineered disulfide mutants, proline mutations, glycine replacement mutations and cavity filling mutants were combined into the combination mutants. The combination mutants were subjected to the thermal stress test at room temperature, 56° C., and 63° C., as shown in Table 31. Notable protein expression and thermal stability improvement were observed for combination mutants.

TABLE 31
Protein expression and thermal stability of hMPV B F
protein mutants comprising engineered disulfide mutations,
proline substitution mutations, glycine replacement
mutations, and/or cavity filling mutations.

| | | Response Shift with hMPV-2 mAb | | |
|---|---|---|---|---|
| | Protein | No | 56° C. stress | 63° C. stress |
| | expression | stress | resistance | resistance |
| Mutant ID | (mg/L) | (nm) | (nm) | (nm) |
| WT Control | 3.7 | 0.820 | 0.122 | 0.017 |
| hMPV117 | 27.9 | 0.723 | 0.640 | 0.000 |
| hMPV118 | 12.6 | 0.844 | 0.801 | 0.028 |
| hMPV119 | 12.1 | 0.864 | 0.828 | 0.017 |
| hMPV120 | 18.0 | 0.593 | 0.564 | 0.502 |
| hMPV121 | 5.6 | 0.842 | 0.794 | 0.078 |
| hMPV122 | 7.7 | 0.730 | 0.686 | 0.665 |
| hMPV123 | 16.6 | 0.619 | 0.559 | 0.549 |
| hMPV124 | 6.5 | 0.833 | 0.766 | 0.065 |
| hMPV125 | 7.3 | 0.847 | 0.785 | 0.233 |
| hMPV126 | 19.5 | 0.773 | 0.730 | −0.075 |
| hMPV127 | 8.0 | 0.719 | 0.674 | 0.696 |
| hMPV128 | 9.6 | 0.777 | 0.769 | 0.746 |
| hMPV129 | 15.3 | 0.687 | 0.632 | 0.661 |
| hMPV130 | 23.6 | 0.710 | 0.620 | 0.079 |
| hMPV131 | 10.9 | 0.727 | 0.657 | 0.679 |
| hMPV132 | 20.1 | 0.704 | 0.694 | 0.606 |
| hMPV133 | 34.6 | 0.677 | 0.616 | 0.620 |
| hMPV134 | 10.9 | 0.718 | 0.665 | 0.733 |
| hMPV135 | 13.2 | 0.730 | 0.724 | −0.015 |
| hMPV136 | 21.7 | 0.591 | 0.541 | −0.027 |
| hMPV137 | 11.8 | 0.663 | 0.626 | 0.238 |
| hMPV138 | 21.7 | 0.681 | 0.663 | 0.156 |
| hMPV139 | 19.1 | 0.699 | 0.650 | 0.456 |
| hMPV140 | 8.5 | 0.731 | 0.660 | 0.747 |
| hMPV142 | 10.9 | 0.731 | 0.706 | 0.054 |
| hMPV144 | 20.7 | 0.712 | 0.670 | −0.015 |

### Example 9: Design and Preparation of PIV1 F Protein Mutants

This example illustrates the design and preparation of various PIV1 F protein mutants, which include a fibritin foldon trimerization domain and introduced amino acid mutations, such as engineered disulfide bond mutations, cavity filling mutations, proline substitution mutations, glycine replacement mutations, cleavage site mutations, or a combination thereof. Exemplary PIV1 F protein mutants, each of which is identified by a unique identifier, such as PIV1014, PIV1039, etc., are provided in Tables 32-37. Each of these mutants was designed and prepared based on the amino acid sequence set forth in SEQ ID NO:211. Amino acid residues 1-477 of the sequence of SEQ ID NO:211 are identical to amino acid residues 1-477 of the F0 precursor polypeptide of native PIV as set forth in SEQ ID NO:206. Therefore, the amino acid sequences of these exemplary F protein mutants are identical except for the introduced amino acid mutations as noted for each mutant listed in Tables 32-37. Each of these PIV1 F protein mutants comprises two separate polypeptide chains. One of the polypeptide chains, the F2 polypeptide, comprises amino acids 22-112 of SEQ ID NO:211 except for the introduced mutations as noted. The other polypeptide chain comprises the F1 polypeptide (residues 113-477) linked to a foldon trimerization domain (residues 482-508) via a GGGS linker (residues 478-481). The signal peptide (residues 1-21) of SEQ ID NO:211 were cleaved from the F0 precursor during the expression process.

TABLE 32
Exemplary PIV1 F Protein Mutants Comprising
Engineered Disulfide Mutations

| | | Amino Acid Sequence (residues |
|---|---|---|
| | | 113-477 for F1 polypeptide and |
| Mutant | Muta- | residues 22-112 for F2 |
| ID | tions | polypeptide) |
| PIV | Q92C- | F1 (SEQ ID NO: 212): |
| 1014 | G134C | FFGAVIGTIALGVATAAQITACIALAEAREARKDI |
| | | ALIKDSIVKTHNSVEFIQRGIGEQIIALKTLQDFV |
| | | NDEIRPAIGELRCETTALKLGIKLTQHYSELATAF |
| | | SSNLGTIGEKSLTLQALSSLYSANITEILSTIKKD |
| | | KSDIYDIIYTEQVKGTVIDVDLEKYMVTLLVKIPI |
| | | LSEIPGVLIYRASSISYNIEGEEWHVAIPNYIINK |
| | | ASSLGGADVTNCIESKLAYICPRDPTQLIPDNQQK |
| | | CILGDVSKCPVTKVINNLVPKFAFINGGVVANCIA |
| | | STCTCGTNRIPVNQDRSKGVTFLTYTNCGLIGING |
| | | IELYANKRGRDTTWGNQIIKVGPAVSIRPVDISLN |
| | | LASATNFLEESKTEL |
| | | F2 (SEQ ID NO: 213): |
| | | QIPVDKLSNVGVIINEGKLLKIAGSYESRYIVLSL |
| | | VPSIDLQDGCGTTQIIQYKNLLNRLLIPLKDALDL |
| | | CESLITITNDTTVTNDNPQTR |

TABLE 33
Exemplary PIV1 F Protein Mutants Comprising
Proline Substitution Mutations

| | | Amino Acid Sequence (residues |
|---|---|---|
| | | 113-477 for F1 polypeptide and |
| Mutant | Muta- | residues 22-112 for F2 |
| ID | tions | polypeptide) |
| PIV1039 | A128P | F1 (SEQ ID NO: 214): |
| | | FFGAVIGTIALGVATPAQITAGIALAEAREARKDI |
| | | ALIKDSIVKTHNSVEFIQRGIGEQIIALKTLQDFV |
| | | NDEIRPAIGELRCETTALKLGIKLTQHYSELATAF |
| | | SSNLGTIGEKSLTLQALSSLYSANITEILSTIKKD |
| | | KSDIYDIIYTEQVKGTVIDVDLEKYMVTLLVKIPI |
| | | LSEIPGVLIYRASSISYNIEGEEWHVAIPNYIINK |
| | | ASSLGGADVTNCIESKLAYICPRDPTQLIPDNQQK |
| | | CILGDVSKCPVTKVINNLVPKFAFINGGVVANCIA |
| | | STCTCGTNRIPVNQDRSKGVTFLTYTNCGLIGING |
| | | IELYANKRGRDTTWGNQIIKVGPAVSIRPVDISLN |
| | | LASATNFLEESKTEL |
| | | F2 (SEQ ID NO: 215): |
| | | QIPVDKLSNVGVIINEGKLLKIAGSYESRYIVLSL |
| | | VPSIDLQDGCGTTQIIQYKNLLNRLLIPLKDALDL |
| | | QESLITITNDTTVINDNPQTR |

TABLE 34
Exemplary PIV1 F Protein Mutants Comprising Cavity
Filling Mutations

| | | Amino Acid Sequence (residues 113-477 for |
|---|---|---|
| | | F1 polypeptide and residues 22-112 for F2 |
| | | polypeptide. For mutants containing A480L |
| Mutant | | mutation, residues 113-480 for F1 |
| ID | Mutations | polypeptide) |
| PIV1023 | A466L | F1 (SEQ ID NO: 216): |
| | | FFGAVIGTIALGVATAAQITAGIALAEAREARKDIALIKDSIV |
| | | KTHNSVEFIQRGIGEQIIALKTLQDFVNDEIRPAIGELRCET |
| | | TALKLGIKLTQHYSELATAFSSNLGTIGEKSLTLQALSSLYS |
| | | ANITEILSTIKKDKSDIYDIIYTEQVKGTVIDVDLEKYMVTLLV |
| | | KIPILSEIPGVLIYRASSISYNIEGEEWHVAIPNYIINKASSLG |
| | | GADVTNCIESKLAYICPRDPTQLIPDNQQKCILGDVSKCPV |
| | | TKVINNLVPKFAFINGGVVANCIASTCTCGTNRIPVNQDRS |
| | | KGVTFLTYTNCGLIGINGIELYANKRGRDTTWGNQIIKVGP |
| | | AVSIRPVDISLNLASLTNFLEESKTEL |
| | | F2 (SEQ ID NO: 217): |
| | | QIPVDKLSNVGVIINEGKLLKIAGSYESRYIVLSLVPSIDLQD |
| | | GCGTTQIIQYKNLLNRLLIPLKDALDLQESLITITNDTTVIND |
| | | NPQTR |
| PIV1024 | A466V | F1 (SEQ ID NO: 218): |
| | | FFGAVIGTIALGVATAAQITAGIALAEAREARKDIALIKDSIV |
| | | KTHNSVEFIQRGIGEQIIALKTLQDFVNDEIRPAIGELRCET |
| | | TALKLGIKLTQHYSELATAFSSNLGTIGEKSLTLQALSSLYS |
| | | ANITEILSTIKKDKSDIYDIIYTEQVKGTVIDVDLEKYMVTLLV |
| | | KIPILSEIPGVLIYRASSISYNIEGEEWHVAIPNYIINKASSLG |
| | | GADVTNCIESKLAYICPRDPTQLIPDNQQKCILGDVSKCPV |
| | | TKVINNLVPKFAFINGGVVANCIASTCTCGTNRIPVNQDRS |
| | | KGVTFLTYTNCGLIGINGIELYANKRGRDTTWGNQIIKVGP |
| | | AVSIRPVDISLNLASVTNFLEESKTEL |
| | | F2 (SEQ ID NO: 219): |
| | | QIPVDKLSNVGVIINEGKLLKIAGSYESRYIVLSLVPSIDLQD |
| | | GCGTTQIIQYKNLLNRLLIPLKDALDLQESLITITNDTTVTND |
| | | NPQTR |
| PIV1026 | S473V | F1 (SEQ ID NO: 220): |
| | | FFGAVIGTIALGVATAAQITAGIALAEAREARKDIALIKDSIV |
| | | KTHNSVEFIQRGIGEQIIALKTLQDFVNDEIRPAIGELRCET |
| | | TALKLGIKLTQHYSELATAFSSNLGTIGEKSLTLQALSSLYS |
| | | ANITEILSTIKKDKSDIYDIIYTEQVKGTVIDVDLEKYMVTLLV |
| | | KIPILSEIPGVLIYRASSISYNIEGEEWHVAIPNYIINKASSLG |
| | | GADVTNCIESKLAYICPRDPTQLIPDNQQKCILGDVSKCPV |
| | | TKVINNLVPKFAFINGGVVANCIASTCTCGTNRIPVNQDRS |
| | | KGVTFLTYTNCGLIGINGIELYANKRGRDTTWGNQIIKVGP |
| | | AVSIRPVDISLNLASATNFLEEVKTEL |
| | | F2 (SEQ ID NO: 221): |
| | | QIPVDKLSNVGVIINEGKLLKIAGSYESRYIVLSLVPSIDLQD |
| | | GCGTTQIIQYKNLLNRLLIPLKDALDLQESLITITNDTTVIND |
| | | NPQTR |
| PIV1027 | S473L | F1 (SEQ ID NO: 222): |
| | | FFGAVIGTIALGVATAAQITAGIALAEAREARKDIALIKDSIV |
| | | KTHNSVEFIQRGIGEQIIALKTLQDFVNDEIRPAIGELRCET |
| | | TALKLGIKLTQHYSELATAFSSNLGTIGEKSLTLQALSSLYS |
| | | ANITEILSTIKKDKSDIYDIIYTEQVKGTVIDVDLEKYMVTLLV |
| | | KIPILSEIPGVLIYRASSISYNIEGEEWHVAIPNYIINKASSLG |
| | | GADVTNCIESKLAYICPRDPTQLIPDNQQKCILGDVSKCPV |
| | | TKVINNLVPKFAFINGGVVANCIASTCTCGTNRIPVNQDRS |
| | | KGVTFLTYTNCGLIGINGIELYANKRGRDTTWGNQIIKVGP |
| | | AVSIRPVDISLNLASATNFLEELKTEL |
| | | F2 (SEQ ID NO: 223): |
| | | QIPVDKLSNVGVIINEGKLLKIAGSYESRYIVLSLVPSIDLQD |
| | | GCGTTQIIQYKNLLNRLLIPLKDALDLQESLITITNDTTVTND |
| | | NPQTR |
| PIV1028 | A480L | F1 (SEQ ID NO: 224): |
| | | FFGAVIGTIALGVATAAQITAGIALAEAREARKDIALIKDSIV |
| | | KTHNSVEFIQRGIGEQIIALKTLQDFVNDEIRPAIGELRCET |
| | | TALKLGIKLTQHYSELATAFSSNLGTIGEKSLTLQALSSLYS |
| | | ANITEILSTIKKDKSDIYDIIYTEQVKGTVIDVDLEKYMVTLLV |
| | | KIPILSEIPGVLIYRASSISYNIEGEEWHVAIPNYIINKASSLG |
| | | GADVTNCIESKLAYICPRDPTQLIPDNQQKCILGDVSKCPV |
| | | TKVINNLVPKFAFINGGVVANCIASTCTCGTNRIPVNQDRS |
| | | KGVTFLTYTNCGLIGINGIELYANKRGRDTTWGNQIIKVGP |
| | | AVSIRPVDISLNLASATNFLEESKTELMKL |
| | | F2 (SEQ ID NO: 225): |
| | | QIPVDKLSNVGVIINEGKLLKIAGSYESRYIVLSLVPSIDLQD |
| | | GCGTTQIIQYKNLLNRLLIPLKDALDLQESLITITNDTTVIND |
| | | NPQTR |
| PIV1029 | A480V | F1 (SEQ ID NO: 226) |
| | | FFGAVIGTIALGVATAAQITAGIALAEAREARKDIALIKDSIV |
| | | KTHNSVEFIQRGIGEQIIALKTLQDFVNDEIRPAIGELRCET |
| | | TALKLGIKLTQHYSELATAFSSNLGTIGEKSLTLQALSSLYS |
| | | ANITEILSTIKKDKSDIYDIIYTEQVKGTVIDVDLEKYMVTLLV |
| | | KIPILSEIPGVLIYRASSISYNIEGEEWHVAIPNYIINKASSLG |
| | | GADVTNCIESKLAYICPRDPTQLIPDNQQKCILGDVSKCPV |
| | | TKVINNLVPKFAFINGGVVANCIASTCTCGTNRIPVNQDRS |
| | | KGVTFLTYTNCGLIGINGIELYANKRGRDTTWGNQIIKVGP |
| | | AVSIRPVDISLNLASATNFLEESKTELMKV |
| | | F2 (SEQ ID NO: 227): |
| | | QIPVDKLSNVGVIINEGKLLKIAGSYESRYIVLSLVPSIDLQD |
| | | GCGTTQIIQYKNLLNRLLIPLKDALDLQESLITITNDTTVTND |
| | | NPQTR |
| PIV1030 | A466L, | F1 (SEQ ID NO: 228): |
| | S473A | FFGAVIGTIALGVATAAQITAGIALAEAREARKDIALIKDSIV |
| | | KTHNSVEFIQRGIGEQIIALKTLQDFVNDEIRPAIGELRCET |
| | | TALKLGIKLTQHYSELATAFSSNLGTIGEKSLTLQALSSLYS |
| | | ANITEILSTIKKDKSDIYDIIYTEQVKGTVIDVDLEKYMVTLLV |
| | | KIPILSEIPGVLIYRASSISYNIEGEEWHVAIPNYIINKASSLG |
| | | GADVTNCIESKLAYICPRDPTQLIPDNQQKCILGDVSKCPV |
| | | TKVINNLVPKFAFINGGVVANCIASTCTCGTNRIPVNQDRS |
| | | KGVTFLTYTNCGLIGINGIELYANKRGRDTTWGNQIIKVGP |
| | | AVSIRPVDISLNLASLTNFLEEAKTEL |
| | | F2 (SEQ ID NO: 229): |
| | | QIPVDKLSNVGVIINEGKLLKIAGSYESRYIVLSLVPSIDLQD |
| | | GCGTTQIIQYKNLLNRLLIPLKDALDLQESLITITNDTTVTND |
| | | NPQTR |
| PIV1031 | A466L, | F1 (SEQ ID NO: 230): |
| | S473L | FFGAVIGTIALGVATAAQITAGIALAEAREARKDIALIKDSIV |
| | | KTHNSVEFIQRGIGEQIIALKTLQDFVNDEIRPAIGELRCET |
| | | TALKLGIKLTQHYSELATAFSSNLGTIGEKSLTLQALSSLYS |
| | | ANITEILSTIKKDKSDIYDIIYTEQVKGTVIDVDLEKYMVTLLV |
| | | KIPILSEIPGVLIYRASSISYNIEGEEWHVAIPNYIINKASSLG |
| | | GADVTNCIESKLAYICPRDPTQLIPDNQQKCILGDVSKCPV |
| | | TKVINNLVPKFAFINGGVVANCIASTCTCGTNRIPVNQDRS |
| | | KGVTFLTYTNCGLIGINGIELYANKRGRDTTWGNQIIKVGP |
| | | AVSIRPVDISLNLASLTNFLEELKTEL |
| | | F2 (SEQ ID NO: 231): |
| | | QIPVDKLSNVGVIINEGKLLKIAGSYESRYIVLSLVPSIDLQD |
| | | GCGTTQIIQYKNLLNRLLIPLKDALDLQESLITITNDTTVIND |
| | | NPQTR |
| PIV1033 | T198A | F1 (SEQ ID NO: 232): |
| | | FFGAVIGTIALGVATAAQITAGIALAEAREARKDIALIKDSIV |
| | | KTHNSVEFIQRGIGEQIIALKTLQDFVNDEIRPAIGELRCET |
| | | AALKLGIKLTQHYSELATAFSSNLGTIGEKSLTLQALSSLYS |
| | | ANITEILSTIKKDKSDIYDIIYTEQVKGTVIDVDLEKYMVTLLV |
| | | KIPILSEIPGVLIYRASSISYNIEGEEWHVAIPNYIINKASSLG |
| | | GADVTNCIESKLAYICPRDPTQLIPDNQQKCILGDVSKCPV |
| | | TKVINNLVPKFAFINGGVVANCIASTCTCGTNRIPVNQDRS |
| | | KGVTFLTYTNCGLIGINGIELYANKRGRDTTWGNQIIKVGP |
| | | AVSIRPVDISLNLASATNFLEESKTEL |
| | | F2 (SEQ ID NO: 233): |
| | | QIPVDKLSNVGVIINEGKLLKIAGSYESRYIVLSLVPSIDLQD |
| | | GCGTTQIIQYKNLLNRLLIPLKDALDLQESLITITNDTTVIND |
| | | NPQTR |
| PIV1042 | Q92A | F1 (SEQ ID NO: 234): |
| | | FFGAVIGTIALGVATAAQITAGIALAEAREARKDIALIKDSIV |
| | | KTHNSVEFIQRGIGEQIIALKTLQDFVNDEIRPAIGELRCET |
| | | TALKLGIKLTQHYSELATAFSSNLGTIGEKSLTLQALSSLYS |
| | | ANITEILSTIKKDKSDIYDIIYTEQVKGTVIDVDLEKYMVTLLV |
| | | KIPILSEIPGVLIYRASSISYNIEGEEWHVAIPNYIINKASSLG |
| | | GADVTNCIESKLAYICPRDPTQLIPDNQQKCILGDVSKCPV |
| | | TKVINNLVPKFAFINGGVVANCIASTCTCGTNRIPVNQDRS |
| | | KGVTFLTYTNCGLIGINGIELYANKRGRDTTWGNQIIKVGP |
| | | AVSIRPVDISLNLASATNFLEESKTEL |
| | | F2 (SEQ ID NO: 235): |
| | | QIPVDKLSNVGVIINEGKLLKIAGSYESRYIVLSLVPSIDLQD |
| | | GCGTTQIIQYKNLLNRLLIPLKDALDLAESLITITNDTTVIND |
| | | NPQTR |
| PIV1043 | Q92A, | F1 (SEQ ID NO: 236): |
| | G134A | FFGAVIGTIALGVATAAQITAAIALAEAREARKDIALIKDSIVK |
| | | THNSVEFIQRGIGEQIIALKTLQDFVNDEIRPAIGELRCETT |
| | | ALKLGIKLTQHYSELATAFSSNLGTIGEKSLTLQALSSLYSA |
| | | NITEILSTIKKDKSDIYDIIYTEQVKGTVIDVDLEKYMVTLLVK |
| | | IPILSEIPGVLIYRASSISYNIEGEEWHVAIPNYIINKASSLGG |
| | | ADVTNCIESKLAYICPRDPTQLIPDNQQKCILGDVSKCPVT |
| | | KVINNLVPKFAFINGGVVANCIASTCTCGTNRIPVNQDRSK |
| | | GVTFLTYTNCGLIGINGIELYANKRGRDTTWGNQIIKVGPA |
| | | VSIRPVDISLNLASATNFLEESKTEL |
| | | F2 (SEQ ID NO: 237): |
| | | QIPVDKLSNVGVIINEGKLLKIAGSYESRYIVLSLVPSIDLQD |
| | | GCGTTQIIQYKNLLNRLLIPLKDALDLAESLITITNDTTVIND |
| | | NPQTR |
| PIV1044 | Q92L | F1 (SEQ ID NO: 238): |
| | | FFGAVIGTIALGVATAAQITAGIALAEAREARKDIALIKDSIV |
| | | KTHNSVEFIQRGIGEQIIALKTLQDFVNDEIRPAIGELRCET |
| | | TALKLGIKLTQHYSELATAFSSNLGTIGEKSLTLQALSSLYS |
| | | ANITEILSTIKKDKSDIYDIIYTEQVKGTVIDVDLEKYMVTLLV |
| | | KIPILSEIPGVLIYRASSISYNIEGEEWHVAIPNYIINKASSLG |
| | | GADVTNCIESKLAYICPRDPTQLIPDNQQKCILGDVSKCPV |
| | | TKVINNLVPKFAFINGGVVANCIASTCTCGTNRIPVNQDRS |
| | | KGVTFLTYTNCGLIGINGIELYANKRGRDTTWGNQIIKVGP |
| | | AVSIRPVDISLNLASATNFLEESKTEL |
| | | F2 (SEQ ID NO: 239): |
| | | QIPVDKLSNVGVIINEGKLLKIAGSYESRYIVLSLVPSIDLQD |
| | | GCGTTQIIQYKNLLNRLLIPLKDALDLLESLITITNDTTVIND |
| | | NPQTR |
| PIV1045 | Q92L, | F1 (SEQ ID NO: 240): |
| | G134A | FFGAVIGTIALGVATAAQITAAIALAEAREARKDIALIKDSIVK |
| | | THNSVEFIQRGIGEQIIALKTLQDFVNDEIRPAIGELRCETT |
| | | ALKLGIKLTQHYSELATAFSSNLGTIGEKSLTLQALSSLYSA |
| | | NITEILSTIKKDKSDIYDIIYTEQVKGTVIDVDLEKYMVTLLVK |
| | | IPILSEIPGVLIYRASSISYNIEGEEWHVAIPNYIINKASSLGG |
| | | ADVTNCIESKLAYICPRDPTQLIPDNQQKCILGDVSKCPVT |
| | | KVINNLVPKFAFINGGVVANCIASTCTCGTNRIPVNQDRSK |
| | | GVTFLTYTNCGLIGINGIELYANKRGRDTTWGNQIIKVGPA |
| | | VSIRPVDISLNLASATNFLEESKTEL |
| | | F2 (SEQ ID NO: 241): |
| | | QIPVDKLSNVGVIINEGKLLKIAGSYESRYIVLSLVPSIDLQD |
| | | GCGTTQIIQYKNLLNRLLIPLKDALDLLESLITITNDTTVIND |
| | | NPQTR |
| PIV1046 | A466V, | F1 (SEQ ID NO: 242): |
| | S473V | FFGAVIGTIALGVATAAQITAGIALAEAREARKDIALIKDSIV |
| | | KTHNSVEFIQRGIGEQIIALKTLQDFVNDEIRPAIGELRCET |
| | | TALKLGIKLTQHYSELATAFSSNLGTIGEKSLTLQALSSLYS |
| | | ANITEILSTIKKDKSDIYDIIYTEQVKGTVIDVDLEKYMVTLLV |
| | | KIPILSEIPGVLIYRASSISYNIEGEEWHVAIPNYIINKASSLG |
| | | GADVTNCIESKLAYICPRDPTQLIPDNQQKCILGDVSKCPV |
| | | TKVINNLVPKFAFINGGVVANCIASTCTCGTNRIPVNQDRS |
| | | KGVTFLTYTNCGLIGINGIELYANKRGRDTTWGNQIIKVGP |
| | | AVSIRPVDISLNLASVTNFLEEVKTEL |
| | | F2 (SEQ ID NO: 243): |
| | | QIPVDKLSNVGVIINEGKLLKIAGSYESRYIVLSLVPSIDLQD |
| | | GCGTTQIIQYKNLLNRLLIPLKDALDLQESLITITNDTTVTND |
| | | NPQTR |

TABLE 35
Exemplary PIV1 F Protein Mutants Comprising Glycine Replacement Mutation

| | | Amino Acid Sequence (residues 113-477 for F1 |
|---|---|---|
| Mutant ID | Mutations | polypeptide and residues 22-112 for F2 polypeptide) |
| PIV1036 | G134A | F1 (SEQ ID NO: 244): |
| | | FFGAVIGTIALGVATAAQITAAIALAEAREARKDIALIKDSIVK |
| | | THNSVEFIQRGIGEQIIALKTLQDFVNDEIRPAIGELRCETT |
| | | ALKLGIKLTQHYSELATAFSSNLGTIGEKSLTLQALSSLYSA |
| | | NITEILSTIKKDKSDIYDIIYTEQVKGTVIDVDLEKYMVTLLVK |
| | | IPILSEIPGVLIYRASSISYNIEGEEWHVAIPNYIINKASSLGG |
| | | ADVTNCIESKLAYICPRDPTQLIPDNQQKCILGDVSKCPVT |
| | | KVINNLVPKFAFINGGVVANCIASTCTCGTNRIPVNQDRSK |
| | | GVTFLTYTNCGLIGINGIELYANKRGRDTTWGNQIIKVGPA |
| | | VSIRPVDISLNLASATNFLEESKTEL |
| | | F2 (SEQ ID NO: 245): |
| | | QIPVDKLSNVGVIINEGKLLKIAGSYESRYIVLSLVPSIDLQD |
| | | GCGTTQIIQYKNLLNRLLIPLKDALDLQESLITITNDTTVTND |
| | | NPQTR |

TABLE 36
Exemplary PIV1 F Protein Mutants Comprising Engineered Disulfide Mutations and
Hydrophobic Filling Mutations

| | | Amino Acid Sequence (residues 113-477 for F1 |
|---|---|---|
| | | polypeptide and residues 22-112 for F2 |
| Mutant | | polypeptide. For mutants containing |
| ID | Mutations | A480L mutation, residues 113-480 for F1 polypeptide) |
| PIV1048 | Q92C-G134C, | F1 (SEQ ID NO: 246): |
| | A466L, S473L, | FFGAVIGTIALGVATAAQITACIALAEAREARKDIALIKDSIVKT |
| | A480L | HNSVEFIQRGIGEQIIALKTLQDFVNDEIRPAIGELRCETTAL |
| | | KLGIKLTQHYSELATAFSSNLGTIGEKSLTLQALSSLYSANIT |
| | | EILSTIKKDKSDIYDIIYTEQVKGTVIDVDLEKYMVTLLVKIPIL |
| | | SEIPGVLIYRASSISYNIEGEEWHVAIPNYIINKASSLGGADV |
| | | TNCIESKLAYICPRDPTQLIPDNQQKCILGDVSKCPVTKVINN |
| | | LVPKFAFINGGVVANCIASTCTCGTNRIPVNQDRSKGVTFLT |
| | | YTNCGLIGINGIELYANKRGRDTTWGNQIIKVGPAVSIRPVDI |
| | | SLNLASLTNFLEELKTELMKL |
| | | F2 (SEQ ID NO: 247): |
| | | QIPVDKLSNVGVIINEGKLLKIAGSYESRYIVLSLVPSIDLQDG |
| | | CGTTQIIQYKNLLNRLLIPLKDALDLCESLITITNDTTVTNDNP |
| | | QTR |
| PIV1049 | Q92C-G134C, | F1 (SEQ ID NO: 248): |
| | A466L, S473L | FFGAVIGTIALGVATAAQITACIALAEAREARKDIALIKDSIVKT |
| | | HNSVEFIQRGIGEQIIALKTLQDFVNDEIRPAIGELRCETTAL |
| | | KLGIKLTQHYSELATAFSSNLGTIGEKSLTLQALSSLYSANIT |
| | | EILSTIKKDKSDIYDIIYTEQVKGTVIDVDLEKYMVTLLVKIPIL |
| | | SEIPGVLIYRASSISYNIEGEEWHVAIPNYIINKASSLGGADV |
| | | TNCIESKLAYICPRDPTQLIPDNQQKCILGDVSKCPVTKVINN |
| | | LVPKFAFINGGVVANCIASTCTCGTNRIPVNQDRSKGVTFLT |
| | | YTNCGLIGINGIELYANKRGRDTTWGNQIIKVGPAVSIRPVDI |
| | | SLNLASLTNFLEELKTEL |
| | | F2 (SEQ ID NO: 249): |
| | | QIPVDKLSNVGVIINEGKLLKIAGSYESRYIVLSLVPSIDLQDG |
| | | CGTTQIIQYKNLLNRLLIPLKDALDLCESLITITNDTTVTNDNP |
| | | QTR |
| PIV1051 | Q92C-G134C, | F1 (SEQ ID NO: 250): |
| | A466V, | FFGAVIGTIALGVATAAQITACIALAEAREARKDIALIKDSIVKT |
| | S473V, A480V | HNSVEFIQRGIGEQIIALKTLQDFVNDEIRPAIGELRCETTAL |
| | | KLGIKLTQHYSELATAFSSNLGTIGEKSLTLQALSSLYSANIT |
| | | EILSTIKKDKSDIYDIIYTEQVKGTVIDVDLEKYMVTLLVKIPIL |
| | | SEIPGVLIYRASSISYNIEGEEWHVAIPNYIINKASSLGGADV |
| | | TNCIESKLAYICPRDPTQLIPDNQQKCILGDVSKCPVTKVINN |
| | | LVPKFAFINGGVVANCIASTCTCGTNRIPVNQDRSKGVTFLT |
| | | YTNCGLIGINGIELYANKRGRDTTWGNQIIKVGPAVSIRPVDI |
| | | SLNLASVTNFLEEVKTELMKV |
| | | F2 (SEQ ID NO: 251): |
| | | QIPVDKLSNVGVIINEGKLLKIAGSYESRYIVLSLVPSIDLQDG |
| | | CGTTQIIQYKNLLNRLLIPLKDALDLCESLITITNDTTVTNDNP |
| | | QTR |
| PIV1052 | Q92C-G134C, | F1 (SEQ ID NO: 252): |
| | A466V, S473V | FFGAVIGTIALGVATAAQITACIALAEAREARKDIALIKDSIVKT |
| | | HNSVEFIQRGIGEQIIALKTLQDFVNDEIRPAIGELRCETTAL |
| | | KLGIKLTQHYSELATAFSSNLGTIGEKSLTLQALSSLYSANIT |
| | | EILSTIKKDKSDIYDIIYTEQVKGTVIDVDLEKYMVTLLVKIPIL |
| | | SEIPGVLIYRASSISYNIEGEEWHVAIPNYIINKASSLGGADV |
| | | TNCIESKLAYICPRDPTQLIPDNQQKCILGDVSKCPVTKVINN |
| | | LVPKFAFINGGVVANCIASTCTCGTNRIPVNQDRSKGVTFLT |
| | | YTNCGLIGINGIELYANKRGRDTTWGNQIIKVGPAVSIRPVDI |
| | | SLNLASVTNFLEEVKTEL |
| | | F2 (SEQ ID NO: 253): |
| | | QIPVDKLSNVGVIINEGKLLKIAGSYESRYIVLSLVPSIDLQDG |
| | | CGTTQIIQYKNLLNRLLIPLKDALDLCESLITITNDTTVTNDNP |
| | | QTR |
| PIV1054 | A466L, S473L, | F1 (SEQ ID NO: 254): |
| | A480L, G134A | FFGAVIGTIALGVATAAQITAAIALAEAREARKDIALIKDSIVKT |
| | | HNSVEFIQRGIGEQIIALKTLQDFVNDEIRPAIGELRCETTAL |
| | | KLGIKLTQHYSELATAFSSNLGTIGEKSLTLQALSSLYSANIT |
| | | EILSTIKKDKSDIYDIIYTEQVKGTVIDVDLEKYMVTLLVKIPIL |
| | | SEIPGVLIYRASSISYNIEGEEWHVAIPNYIINKASSLGGADV |
| | | TNCIESKLAYICPRDPTQLIPDNQQKCILGDVSKCPVTKVINN |
| | | LVPKFAFINGGVVANCIASTCTCGTNRIPVNQDRSKGVTFLT |
| | | YTNCGLIGINGIELYANKRGRDTTWGNQIIKVGPAVSIRPVDI |
| | | SLNLASLTNFLEELKTELMKL |
| | | F2 (SEQ ID NO: 255): |
| | | QIPVDKLSNVGVIINEGKLLKIAGSYESRYIVLSLVPSIDLQDG |
| | | CGTTQIIQYKNLLNRLLIPLKDALDLQESLITITNDTTVTNDNP |
| | | QTR |
| PIV1055 | A466L, S473L, | F1 (SEQ ID NO: 256): |
| | G134A | FFGAVIGTIALGVATAAQITAAIALAEAREARKDIALIKDSIVKT |
| | | HNSVEFIQRGIGEQIIALKTLQDFVNDEIRPAIGELRCETTAL |
| | | KLGIKLTQHYSELATAFSSNLGTIGEKSLTLQALSSLYSANIT |
| | | EILSTIKKDKSDIYDIIYTEQVKGTVIDVDLEKYMVTLLVKIPIL |
| | | SEIPGVLIYRASSISYNIEGEEWHVAIPNYIINKASSLGGADV |
| | | TNCIESKLAYICPRDPTQLIPDNQQKCILGDVSKCPVTKVINN |
| | | LVPKFAFINGGVVANCIASTCTCGTNRIPVNQDRSKGVTFLT |
| | | YTNCGLIGINGIELYANKRGRDTTWGNQIIKVGPAVSIRPVDI |
| | | SLNLASLTNFLEELKTEL |
| | | F2 (SEQ ID NO: 257): |
| | | QIPVDKLSNVGVIINEGKLLKIAGSYESRYIVLSLVPSIDLQDG |
| | | CGTTQIIQYKNLLNRLLIPLKDALDLQESLITITNDTTVTNDNP |
| | | QTR |
| PIV1057 | A466L, S473L, | F1 (SEQ ID NO: 258): |
| | A480L, Q92A, | FFGAVIGTIALGVATAAQITAAIALAEAREARKDIALIKDSIVKT |
| | G134A | HNSVEFIQRGIGEQIIALKTLQDFVNDEIRPAIGELRCETTAL |
| | | KLGIKLTQHYSELATAFSSNLGTIGEKSLTLQALSSLYSANIT |
| | | EILSTIKKDKSDIYDIIYTEQVKGTVIDVDLEKYMVTLLVKIPIL |
| | | SEIPGVLIYRASSISYNIEGEEWHVAIPNYIINKASSLGGADV |
| | | TNCIESKLAYICPRDPTQLIPDNQQKCILGDVSKCPVTKVINN |
| | | LVPKFAFINGGVVANCIASTCTCGTNRIPVNQDRSKGVTFLT |
| | | YTNCGLIGINGIELYANKRGRDTTWGNQIIKVGPAVSIRPVDI |
| | | SLNLASLTNFLEELKTELMKL |
| | | F2 (SEQ ID NO: 259): |
| | | QIPVDKLSNVGVIINEGKLLKIAGSYESRYIVLSLVPSIDLQDG |
| | | CGTTQIIQYKNLLNRLLIPLKDALDLAESLITITNDTTVTNDNP |
| | | QTR |
| PIV1058 | A466L, S473L, | F1 (SEQ ID NO: 260): |
| | Q92A, G134A | FFGAVIGTIALGVATAAQITAAIALAEAREARKDIALIKDSIVKT |
| | | HNSVEFIQRGIGEQIIALKTLQDFVNDEIRPAIGELRCETTAL |
| | | KLGIKLTQHYSELATAFSSNLGTIGEKSLTLQALSSLYSANIT |
| | | EILSTIKKDKSDIYDIIYTEQVKGTVIDVDLEKYMVTLLVKIPIL |
| | | SEIPGVLIYRASSISYNIEGEEWHVAIPNYIINKASSLGGADV |
| | | TNCIESKLAYICPRDPTQLIPDNQQKCILGDVSKCPVTKVINN |
| | | LVPKFAFINGGVVANCIASTCTCGTNRIPVNQDRSKGVTFLT |
| | | YTNCGLIGINGIELYANKRGRDTTWGNQIIKVGPAVSIRPVDI |
| | | SLNLASLTNFLEELKTEL |
| | | F2 (SEQ ID NO: 261): |
| | | QIPVDKLSNVGVIINEGKLLKIAGSYESRYIVLSLVPSIDLQDG |
| | | CGTTQIIQYKNLLNRLLIPLKDALDLAESLITITNDTTVTNDNP |
| | | QTR |
| PIV1060 | Q92L, G134A, | F1 (SEQ ID NO: 262): |
| | A466L, S473L, | FFGAVIGTIALGVATAAQITAAIALAEAREARKDIALIKDSIVKT |
| | A480L | HNSVEFIQRGIGEQIIALKTLQDFVNDEIRPAIGELRCETTAL |
| | | KLGIKLTQHYSELATAFSSNLGTIGEKSLTLQALSSLYSANIT |
| | | EILSTIKKDKSDIYDIIYTEQVKGTVIDVDLEKYMVTLLVKIPIL |
| | | SEIPGVLIYRASSISYNIEGEEWHVAIPNYIINKASSLGGADV |
| | | TNCIESKLAYICPRDPTQLIPDNQQKCILGDVSKCPVTKVINN |
| | | LVPKFAFINGGVVANCIASTCTCGTNRIPVNQDRSKGVTFLT |
| | | YTNCGLIGINGIELYANKRGRDTTWGNQIIKVGPAVSIRPVDI |
| | | SLNLASLTNFLEELKTELMKL |
| | | F2 (SEQ ID NO: 263): |
| | | QIPVDKLSNVGVIINEGKLLKIAGSYESRYIVLSLVPSIDLQDG |
| | | CGTTQIIQYKNLLNRLLIPLKDALDLLESLITITNDTTVTNDNP |
| | | QTR |
| PIV1061 | Q92L, G134A, | F1 (SEQ ID NO: 264): |
| | A466L, S473L | FFGAVIGTIALGVATAAQITAAIALAEAREARKDIALIKDSIVKT |
| | | HNSVEFIQRGIGEQIIALKTLQDFVNDEIRPAIGELRCETTAL |
| | | KLGIKLTQHYSELATAFSSNLGTIGEKSLTLQALSSLYSANIT |
| | | EILSTIKKDKSDIYDIIYTEQVKGTVIDVDLEKYMVTLLVKIPIL |
| | | SEIPGVLIYRASSISYNIEGEEWHVAIPNYIINKASSLGGADV |
| | | TNCIESKLAYICPRDPTQLIPDNQQKCILGDVSKCPVTKVINN |
| | | LVPKFAFINGGVVANCIASTCTCGTNRIPVNQDRSKGVTFLT |
| | | YTNCGLIGINGIELYANKRGRDTTWGNQIIKVGPAVSIRPVDI |
| | | SLNLASLTNFLEELKTEL |
| | | F2 (SEQ ID NO: 265): |
| | | QIPVDKLSNVGVIINEGKLLKIAGSYESRYIVLSLVPSIDLQDG |
| | | CGTTQIIQYKNLLNRLLIPLKDALDLLESLITITNDTTVTNDNP |
| | | QTR |
| PIV1063 | A466L, S473L, | F1 (SEQ ID NO: 266): |
| | A480L, Q92A, | FFGAVIGTIALGVATAAQITALIALAEAREARKDIALIKDSIVKT |
| | G134L | HNSVEFIQRGIGEQIIALKTLQDFVNDEIRPAIGELRCETTAL |
| | | KLGIKLTQHYSELATAFSSNLGTIGEKSLTLQALSSLYSANIT |
| | | EILSTIKKDKSDIYDIIYTEQVKGTVIDVDLEKYMVTLLVKIPIL |
| | | SEIPGVLIYRASSISYNIEGEEWHVAIPNYIINKASSLGGADV |
| | | TNCIESKLAYICPRDPTQLIPDNQQKCILGDVSKCPVTKVINN |
| | | LVPKFAFINGGVVANCIASTCTCGTNRIPVNQDRSKGVTFLT |
| | | YTNCGLIGINGIELYANKRGRDTTWGNQIIKVGPAVSIRPVDI |
| | | SLNLASLTNFLEELKTELMKLF2 (SEQ ID NO: 267): |
| | | QIPVDKLSNVGVIINEGKLLKIAGSYESRYIVLSLVPSIDLQDG |
| | | CGTTQIIQYKNLLNRLLIPLKDALDLAESLITITNDTTVTNDNP |
| | | QTR |
| PIV1066 | A4661, S4731, | F1 (SEQ ID NO: 268): |
| | A480L, Q92L, | FFGAVIGTIALGVATAAQITAAIALAEAREARKDIALIKDSIVKT |
| | G134A | HNSVEFIQRGIGEQIIALKTLQDFVNDEIRPAIGELRCETTAL |
| | | KLGIKLTQHYSELATAFSSNLGTIGEKSLTLQALSSLYSANIT |
| | | EILSTIKKDKSDIYDIIYTEQVKGTVIDVDLEKYMVTLLVKIPIL |
| | | SEIPGVLIYRASSISYNIEGEEWHVAIPNYIINKASSLGGADV |
| | | TNCIESKLAYICPRDPTQLIPDNQQKCILGDVSKCPVTKVINN |
| | | LVPKFAFINGGVVANCIASTCTCGTNRIPVNQDRSKGVTFLT |
| | | YTNCGLIGINGIELYANKRGRDTTWGNQIIKVGPAVSIRPVDI |
| | | SLNLASITNFLEEIKTELMKL |
| | | F2 (SEQ ID NO: 269): |
| | | QIPVDKLSNVGVIINEGKLLKIAGSYESRYIVLSLVPSIDLQDG |
| | | CGTTQIIQYKNLLNRLLIPLKDALDLLESLITITNDTTVINDNP |
| | | QTR |
| PIV1068 | A4661, S4731, | F1 (SEQ ID NO: 270): |
| | A4801, Q92L, | FFGAVIGTIALGVATAAQITAAIALAEAREARKDIALIKDSIVKT |
| | G134A | HNSVEFIQRGIGEQIIALKTLQDFVNDEIRPAIGELRCETTAL |
| | | KLGIKLTQHYSELATAFSSNLGTIGEKSLTLQALSSLYSANIT |
| | | EILSTIKKDKSDIYDIIYTEQVKGTVIDVDLEKYMVTLLVKIPIL |
| | | SEIPGVLIYRASSISYNIEGEEWHVAIPNYIINKASSLGGADV |
| | | TNCIESKLAYICPRDPTQLIPDNQQKCILGDVSKCPVTKVINN |
| | | LVPKFAFINGGVVANCIASTCTCGTNRIPVNQDRSKGVTFLT |
| | | YTNCGLIGINGIELYANKRGRDTTWGNQIIKVGPAVSIRPVDI |
| | | SLNLASITNFLEEIKTELMKI |
| | | F2 (SEQ ID NO: 271): |
| | | QIPVDKLSNVGVIINEGKLLKIAGSYESRYIVLSLVPSIDLQDG |
| | | CGTTQIIQYKNLLNRLLIPLKDALDLLESLITITNDTTVTNDNP |
| | | QTR |

TABLE 37
Exemplary PIV1 F Protein Mutants Comprising Engineered Disulfide Mutations,
Cavity Filling Mutations, and Cleavage Site Mutations

| Mutant | | Amino Acid Sequence (residues 113-477 for F1 |
|---|---|---|
| ID | Mutations | polypeptide and residues 22-112 for F2 polypeptide) |
| PIV1047 | F113G, F114S, | F1 (SEQ ID NO: 272): |
| | Q92C-G134C, | GSGAVIGTIALGVATAAQITACIALAEAREARKDIALIKDSIV |
| | A466L, S473L, | KTHNSVEFIQRGIGEQIIALKTLQDFVNDEIRPAIGELRCET |
| | A480L | TALKLGIKLTQHYSELATAFSSNLGTIGEKSLTLQALSSLYS |
| | | ANITEILSTIKKDKSDIYDIIYTEQVKGTVIDVDLEKYMVTLLV |
| | | KIPILSEIPGVLIYRASSISYNIEGEEWHVAIPNYIINKASSLG |
| | | GADVTNCIESKLAYICPRDPTQLIPDNQQKCILGDVSKCPV |
| | | TKVINNLVPKFAFINGGVVANCIASTCTCGTNRIPVNQDRS |
| | | KGVTFLTYTNCGLIGINGIELYANKRGRDTTWGNQIIKVGP |
| | | AVSIRPVDISLNLASLTNFLEELKTELMKL |
| | | F2 (SEQ ID NO: 273): |
| | | QIPVDKLSNVGVIINEGKLLKIAGSYESRYIVLSLVPSIDLQD |
| | | GCGTTQIIQYKNLLNRLLIPLKDALDLCESLITITNDTTVIND |
| | | NPQTR |
| PIV1050 | F113G, F114S, | F1 (SEQ ID NO: 274): |
| | Q92C-G134C, | GSGAVIGTIALGVATAAQITACIALAEAREARKDIALIKDSIV |
| | A466V, S473V, | KTHNSVEFIQRGIGEQIIALKTLQDFVNDEIRPAIGELRCET |
| | A480V | TALKLGIKLTQHYSELATAFSSNLGTIGEKSLTLQALSSLYS |
| | | ANITEILSTIKKDKSDIYDIIYTEQVKGTVIDVDLEKYMVTLLV |
| | | KIPILSEIPGVLIYRASSISYNIEGEEWHVAIPNYIINKASSLG |
| | | GADVTNCIESKLAYICPRDPTQLIPDNQQKCILGDVSKCPV |
| | | TKVINNLVPKFAFINGGVVANCIASTCTCGTNRIPVNQDRS |
| | | KGVTFLTYTNCGLIGINGIELYANKRGRDTTWGNQIIKVGP |
| | | AVSIRPVDISLNLASVTNFLEEVKTELMKV |
| | | F2 (SEQ ID NO: 275): |
| | | QIPVDKLSNVGVIINEGKLLKIAGSYESRYIVLSLVPSIDLQD |
| | | GCGTTQIIQYKNLLNRLLIPLKDALDLCESLITITNDTTVIND |
| | | NPQTR |
| PIV1053 | F113G, F114S, | F1 (SEQ ID NO: 276): |
| | A466L, S473L, | GSGAVIGTIALGVATAAQITAAIALAEAREARKDIALIKDSIV |
| | A480L, G134A | KTHNSVEFIQRGIGEQIIALKTLQDFVNDEIRPAIGELRCET |
| | | TALKLGIKLTQHYSELATAFSSNLGTIGEKSLTLQALSSLYS |
| | | ANITEILSTIKKDKSDIYDIIYTEQVKGTVIDVDLEKYMVTLLV |
| | | KIPILSEIPGVLIYRASSISYNIEGEEWHVAIPNYIINKASSLG |
| | | GADVTNCIESKLAYICPRDPTQLIPDNQQKCILGDVSKCPV |
| | | TKVINNLVPKFAFINGGVVANCIASTCTCGTNRIPVNQDRS |
| | | KGVTFLTYTNCGLIGINGIELYANKRGRDTTWGNQIIKVGP |
| | | AVSIRPVDISLNLASLTNFLEELKTELMKL |
| | | F2 (SEQ ID NO: 277): |
| | | QIPVDKLSNVGVIINEGKLLKIAGSYESRYIVLSLVPSIDLQD |
| | | GCGTTQIIQYKNLLNRLLIPLKDALDLQESLITITNDTTVTND |
| | | NPQTR |
| PIV1056 | F113G, F114S, | F1 (SEQ ID NO: 278): |
| | A466L, S473L, | GSGAVIGTIALGVATAAQITAAIALAEAREARKDIALIKDSIV |
| | A480L, Q92A, | KTHNSVEFIQRGIGEQIIALKTLQDFVNDEIRPAIGELRCET |
| | G134A | TALKLGIKLTQHYSELATAFSSNLGTIGEKSLTLQALSSLYS |
| | | ANITEILSTIKKDKSDIYDIIYTEQVKGTVIDVDLEKYMVTLLV |
| | | KIPILSEIPGVLIYRASSISYNIEGEEWHVAIPNYIINKASSLG |
| | | GADVTNCIESKLAYICPRDPTQLIPDNQQKCILGDVSKCPV |
| | | TKVINNLVPKFAFINGGVVANCIASTCTCGTNRIPVNQDRS |
| | | KGVTFLTYTNCGLIGINGIELYANKRGRDTTWGNQIIKVGP |
| | | AVSIRPVDISLNLASLTNFLEELKTELMKL |
| | | F2 (SEQ ID NO: 279): |
| | | QIPVDKLSNVGVIINEGKLLKIAGSYESRYIVLSLVPSIDLQD |
| | | GCGTTQIIQYKNLLNRLLIPLKDALDLAESLITITNDTTVTND |
| | | NPQTR |
| PIV1059 | F113G, F114S, | F1 (SEQ ID NO: 280): |
| | A466L, S473L, | GSGAVIGTIALGVATAAQITAAIALAEAREARKDIALIKDSIV |
| | A480L, G134A, | KTHNSVEFIQRGIGEQIIALKTLQDFVNDEIRPAIGELRCET |
| | Q92L | TALKLGIKLTQHYSELATAFSSNLGTIGEKSLTLQALSSLYS |
| | | ANITEILSTIKKDKSDIYDIIYTEQVKGTVIDVDLEKYMVTLLV |
| | | KIPILSEIPGVLIYRASSISYNIEGEEWHVAIPNYIINKASSLG |
| | | GADVTNCIESKLAYICPRDPTQLIPDNQQKCILGDVSKCPV |
| | | TKVINNLVPKFAFINGGVVANCIASTCTCGTNRIPVNQDRS |
| | | KGVTFLTYTNCGLIGINGIELYANKRGRDTTWGNQIIKVGP |
| | | AVSIRPVDISLNLASLTNFLEELKTELMKL |
| | | F2 (SEQ ID NO: 281): |
| | | QIPVDKLSNVGVIINEGKLLKIAGSYESRYIVLSLVPSIDLQD |
| | | GCGTTQIIQYKNLLNRLLIPLKDALDLLESLITITNDTTVIND |
| | | NPQTR |
| PIV1062 | F113G, F114S, | F1 (SEQ ID NO: 282): |
| | A466L, S473L, | GSGAVIGTIALGVATAAQITALIALAEAREARKDIALIKDSIV |
| | A480L, Q92A, | KTHNSVEFIQRGIGEQIIALKTLQDFVNDEIRPAIGELRCET |
| | G134L | TALKLGIKLTQHYSELATAFSSNLGTIGEKSLTLQALSSLYS |
| | | ANITEILSTIKKDKSDIYDIIYTEQVKGTVIDVDLEKYMVTLLV |
| | | KIPILSEIPGVLIYRASSISYNIEGEEWHVAIPNYIINKASSLG |
| | | GADVTNCIESKLAYICPRDPTQLIPDNQQKCILGDVSKCPV |
| | | TKVINNLVPKFAFINGGVVANCIASTCTCGTNRIPVNQDRS |
| | | KGVTFLTYTNCGLIGINGIELYANKRGRDTTWGNQIIKVGP |
| | | AVSIRPVDISLNLASLTNFLEELKTELMKL |
| | | F2 (SEQ ID NO: 283): |
| | | QIPVDKLSNVGVIINEGKLLKIAGSYESRYIVLSLVPSIDLQD |
| | | GCGTTQIIQYKNLLNRLLIPLKDALDLAESLITITNDTTVIND |
| | | NPQTR |
| PIV1064 | F113G, F114S, | F1 (SEQ ID NO: 284): |
| | Q92C-G134C, | GSGAVIGTIALGVATAAQITACIALAEAREARKDIALIKDSIV |
| | A4661, S4731, | KTHNSVEFIQRGIGEQIIALKTLQDFVNDEIRPAIGELRCET |
| | A480L | TALKLGIKLTQHYSELATAFSSNLGTIGEKSLTLQALSSLYS |
| | | ANITEILSTIKKDKSDIYDIIYTEQVKGTVIDVDLEKYMVTLLV |
| | | KIPILSEIPGVLIYRASSISYNIEGEEWHVAIPNYIINKASSLG |
| | | GADVTNCIESKLAYICPRDPTQLIPDNQQKCILGDVSKCPV |
| | | TKVINNLVPKFAFINGGVVANCIASTCTCGTNRIPVNQDRS |
| | | KGVTFLTYTNCGLIGINGIELYANKRGRDTTWGNQIIKVGP |
| | | AVSIRPVDISLNLASITNFLEEIKTELMKL |
| | | F2 (SEQ ID NO: 285): |
| | | QIPVDKLSNVGVIINEGKLLKIAGSYESRYIVLSLVPSIDLQD |
| | | GCGTTQIIQYKNLLNRLLIPLKDALDLCESLITITNDTTVTND |
| | | NPQTR |
| PIV1065 | F113G, F114S, | F1 (SEQ ID NO: 286): |
| | Q92C-G134C, | GSGAVIGTIALGVATAAQITACIALAEAREARKDIALIKDSIV |
| | A4661, S4731 | KTHNSVEFIQRGIGEQIIALKTLQDFVNDEIRPAIGELRCET |
| | | TALKLGIKLTQHYSELATAFSSNLGTIGEKSLTLQALSSLYS |
| | | ANITEILSTIKKDKSDIYDIIYTEQVKGTVIDVDLEKYMVTLLV |
| | | KIPILSEIPGVLIYRASSISYNIEGEEWHVAIPNYIINKASSLG |
| | | GADVTNCIESKLAYICPRDPTQLIPDNQQKCILGDVSKCPV |
| | | TKVINNLVPKFAFINGGVVANCIASTCTCGTNRIPVNQDRS |
| | | KGVTFLTYTNCGLIGINGIELYANKRGRDTTWGNQIIKVGP |
| | | AVSIRPVDISLNLASITNFLEEIKTEL |
| | | F2 (SEQ ID NO: 287): |
| | | QIPVDKLSNVGVIINEGKLLKIAGSYESRYIVLSLVPSIDLQD |
| | | GCGTTQIIQYKNLLNRLLIPLKDALDLCESLITITNDTTVIND |
| | | NPQTR |
| PIV1067 | F113G, F114S, | F1 (SEQ ID NO: 288): |
| | Q92C-G134C, | GSGAVIGTIALGVATAAQITACIALAEAREARKDIALIKDSIV |
| | A4661, S4731, | KTHNSVEFIQRGIGEQIIALKTLQDFVNDEIRPAIGELRCET |
| | A4801 | TALKLGIKLTQHYSELATAFSSNLGTIGEKSLTLQALSSLYS |
| | | ANITEILSTIKKDKSDIYDIIYTEQVKGTVIDVDLEKYMVTLLV |
| | | KIPILSEIPGVLIYRASSISYNIEGEEWHVAIPNYIINKASSLG |
| | | GADVTNCIESKLAYICPRDPTQLIPDNQQKCILGDVSKCPV |
| | | TKVINNLVPKFAFINGGVVANCIASTCTCGTNRIPVNQDRS |
| | | KGVTFLTYTNCGLIGINGIELYANKRGRDTTWGNQIIKVGP |
| | | AVSIRPVDISLNLASITNFLEEIKTELMKI |
| | | F2 (SEQ ID NO: 289): |
| | | QIPVDKLSNVGVIINEGKLLKIAGSYESRYIVLSLVPSIDLQD |
| | | GCGTTQIIQYKNLLNRLLIPLKDALDLCESLITITNDTTVIND |
| | | NPQTR |
| PIV1069 | F113G, F114S, | F1 (SEQ ID NO: 290): |
| | A466L, S473L, | GSGAVIGTIALGVATAAQITAAIALAEAREARKDIALIKDSIV |
| | G134A | KTHNSVEFIQRGIGEQIIALKTLQDFVNDEIRPAIGELRCET |
| | | TALKLGIKLTQHYSELATAFSSNLGTIGEKSLTLQALSSLYS |
| | | ANITEILSTIKKDKSDIYDIIYTEQVKGTVIDVDLEKYMVTLLV |
| | | KIPILSEIPGVLIYRASSISYNIEGEEWHVAIPNYIINKASSLG |
| | | GADVTNCIESKLAYICPRDPTQLIPDNQQKCILGDVSKCPV |
| | | TKVINNLVPKFAFINGGVVANCIASTCTCGTNRIPVNQDRS |
| | | KGVTFLTYTNCGLIGINGIELYANKRGRDTTWGNQIIKVGP |
| | | AVSIRPVDISLNLASLTNFLEELKTEL |
| | | F2 (SEQ ID NO: 291): |
| | | QIPVDKLSNVGVIINEGKLLKIAGSYESRYIVLSLVPSIDLQD |
| | | GCGTTQIIQYKNLLNRLLIPLKDALDLQESLITITNDTTVTND |
| | | NPQTR |
| PIV1070 | F113G, F114S, | F1 (SEQ ID NO: 292): |
| | A4661, S4731, | GSGAVIGTIALGVATAAQITAAIALAEAREARKDIALIKDSIV |
| | G134A | KTHNSVEFIQRGIGEQIIALKTLQDFVNDEIRPAIGELRCET |
| | | TALKLGIKLTQHYSELATAFSSNLGTIGEKSLTLQALSSLYS |
| | | ANITEILSTIKKDKSDIYDIIYTEQVKGTVIDVDLEKYMVTLLV |
| | | KIPILSEIPGVLIYRASSISYNIEGEEWHVAIPNYIINKASSLG |
| | | GADVTNCIESKLAYICPRDPTQLIPDNQQKCILGDVSKCPV |
| | | TKVINNLVPKFAFINGGVVANCIASTCTCGTNRIPVNQDRS |
| | | KGVTFLTYTNCGLIGINGIELYANKRGRDTTWGNQIIKVGP |
| | | AVSIRPVDISLNLASITNFLEEIKTEL |
| | | F2 (SEQ ID NO: 293): |
| | | QIPVDKLSNVGVIINEGKLLKIAGSYESRYIVLSLVPSIDLQD |
| | | GCGTTQIIQYKNLLNRLLIPLKDALDLQESLITITNDTTVTND |
| | | NPQTR |

### Example 10. PIV1 F Protein Mutant Expression Vector Construction

A nucleic acid molecule encoding the consensus PIV1 F0 polypeptide set forth in SEQ ID NO: 211 was mutated using standard molecular biology techniques to encode a precursor polypeptide for a PIV1 F mutant having the introduced amino acid mutations disclosed in Example 9. The structure and components of the precursor polypeptide are set forth in FIG. 1 and SEQ ID NO: 211. The precursor polypeptide comprises of a signal peptide (residues 1-21), F2 polypeptide (residues 22-112), F1 polypeptide (residues 113-477), T4 fibritin foldon (residues 482-508), PreScission cleavage site (residues 512-519), Strep Tag II (residues 523-530), and linker sequences (residues 478-481, 509-511, 520-522).

The protein sequence of SEQ ID NO: 211 was submitted for mammalian codon optimization and synthesis by Genscript (Piscataway, NJ). The nucleotide sequence was introduced into a commercially available expression vector, pcDNA3.1/Zeo(+) (ThermoFisher Scientific, Waltham, MA) that has been modified to encode the CAG promoter (Yamamura et al., Gene, 108(2), 193-199, 1991) in place of the CMV promoter. Double stranded DNA fragments were purchased from Integrated DNA Technologies (Coralville, IA). DNA fragments of the mutagenized F allele not synthesized were generated and amplified by polymerase chain reaction (PCR) with Phusion Flash High-Fidelity PCR Master Mix (ThermoFisher Scientific). Following purification of the linearized expression vector digested using BamHI and Notl, gene fragments of the mutagenized F allele were inserted into the expression vector with NEBuilder HiFi DNA Assembly Kit (New England Biolabs, Ipswich, MA). The presence of the intended sequence was confirmed by Sanger DNA sequencing. Plasmid DNA for transfection into ExpiCHO cells was purified with the Qiagen Plasmid Plus Midi Kit (Qiagen, Valencia, CA). For all commercial kits or reagents, procedures were performed according to the manufacturer's protocol.

### Example 11. Transfection of PIV1 F Protein Mutants

Protein for PIV1 F protein mutant evaluation was produced by transient transfection of ExpiCHO cells (ThermoFisher Scientific) with DNA constructs assembled and prepared as described in Example 10. Transient transfections were carried out according to the manufacturer's protocol. On day 5 post transfection, the cultures were centrifuged, and supernatants were separated from cell pellets. The crude cell supernatants were used for in vitro assays described herein.

### Example 12. Expression and Conformational Integrity OF PIV1 F Protein Mutants

The OCTET HTX (Sartorius, Gottingen, Germany) instrument was used to evaluate the expression and conformational integrity for each mutant. All measurements were conducted at 30° C. temperature in 96-well black plates (Corning, Corning, NY) with a final volume of 240 μL per well at a constant agitation rate of 1000 RPM.

### 12A: Quantitation of Expression of PIV1 F Protein Mutants

Crude cell supernatant was used to quantitate the expression levels of PIV F protein mutants. Anti-Murine IgG Quantitation (AMQ) Biosensors (Sartorius) were first equilibrated in PB before being dipped into more PB to establish the initial baseline. Biosensors were incubated with a mouse Strep-Tag® II monoclonal antibody (mAb) (Novagen, EMD Millipore Corporation, Temecula, CA before being equilibrated in PB to establish the experimental baseline. The mAb-bound biosensors were then dipped into crude cell culture supernatant for 2.5 minutes. OCTET data analysis software (version 12.2, Sartorius) was used to generate a standard curve from a serially diluted purified protein reference within the same assay. Titers for protein mutants were then determined based on the standard curve.

The results are presented in Table 38 and Table 39. The highest expressing amino acid substitutions identified from the screens of the individual engineered disulfide mutants, proline mutations, glycine replacement mutations and cavity filling mutants were combined into the combination mutants. The protein expression of combination mutants was measured using the same procedure as described above. The results are presented in Table 40.

TABLE 38
Protein expression and thermal stability of PIV1 F protein mutants.

| | | Response Shift with PIV1-8 mAb | | |
|---|---|---|---|---|
| | Protein | No | 50° C. stress | 53° C. stress |
| | expression | stress | resistance | resistance |
| Mutant ID | (mg/L) | (nm) | (nm) | (nm) |
| WT Control | 4.1 | 0.009 | 0.057 | 0.022 |
| PIV1014 | 15.0 | 0.857 | 0.011 | 0.009 |
| PIV1023 | 10.5 | 0.831 | 0.018 | −0.001 |
| PIV1024 | 6.3 | 0.745 | 0.022 | −0.016 |
| PIV1026 | 12.3 | 0.606 | −0.022 | −0.039 |
| PIV1027 | 12.8 | 0.748 | −0.022 | −0.020 |
| PIV1028 | 10.8 | 0.579 | −0.018 | −0.052 |
| PIV1029 | 4.9 | 0.029 | −0.020 | −0.039 |
| PIV1030 | 14.2 | 0.753 | −0.031 | −0.046 |
| PIV1031 | 11.0 | 0.766 | 0.720 | 0.489 |
| PIV1033 | 8.9 | −0.056 | −0.027 | −0.073 |
| PIV1036 | 12.8 | 0.753 | 0.007 | −0.042 |
| PIV1039 | 16.8 | −0.068 | −0.039 | −0.055 |

TABLE 39
Protein expression and thermal stability of PIV1 F protein
mutants with alternative thermal stress conditions.

| | | Response Shift with PIV1-8 mAb | | |
|---|---|---|---|---|
| | Protein | No | 53° C. stress | 56° C. stress |
| | expression | stress | resistance | resistance |
| Mutant ID | (mg/L) | (nm) | (nm) | (nm) |
| WT Control | 9.8 | −0.068 | 0.004 | −0.113 |
| PIV1042 | 4.3 | 0.192 | −0.052 | −0.103 |
| PIV1043 | 10.6 | 0.725 | −0.012 | −0.102 |
| PIV1044 | 0.0 | 0.162 | −0.007 | −0.122 |
| PIV1045 | 10.1 | 0.635 | −0.054 | −0.095 |
| PIV1046 | 10.8 | 0.584 | −0.059 | −0.091 |

### 12B: Conformational Integrity of PIV1 F Protein Mutants

Conformation integrity of PIV1 F protein mutants were evaluated by a thermal stress experiment. Crude cell culture supernatants were normalized based on the results of the titer quantitation and then incubated for 1 hour at room temperature and two higher temperatures. For the initial testing of PIV1 mutants, the two temperatures were 50° C. and 53° C. After the incubation, the protein mutants were probed with prefusion specific monoclonal antibody PIV1-8 by OCTET HTX. Anti-Human IgG Fc Capture (AHC) biosensors were first equilibrated in PB for before being dipped into more PB to establish the initial baseline. Biosensors were incubated with PIV1-8 at before being equilibrated in PB to establish the experimental baseline. The mAb-bound biosensors were then dipped into the thermal stresses cell culture supernatants for 5 minutes. The kinetics analysis was done by OCTET data analysis software (version 12.2, Sartorius) based on curve fitting of the entire associate step.

The reactivities of the mutants to PIV1-8 mAb are presented in Table 40 and 41. The most stabilizing amino acid substitutions identified from the screens of the individual engineered disulfide mutants, proline mutations, glycine replacement mutations and cavity filling mutants were combined into the combination mutants. The combination mutants were subjected to the thermal stress test at room temperature, 53° C., and 56° C., as shown in Table 40. Additionally, a known literature prefusion construct was included (“literature prefusion comparison”, F113G, F114S, A4661, S4731, Stewart-Jones et al., 2018, PNAS 115, 12265-12270 (2018)) as a comparison group to the combination mutants generated. Notable protein expression and thermal stability improvement were observed for combination mutants. After thermal stress at 56° C., almost all tested constructs, with the exception of PV1052 and PIV1063, retained higher reactivity to PIV1-8 mAb compared with both WT control and literature prefusion comparison.

TABLE 40
Protein expression and thermal stability of PIV1 F
protein mutants comprising engineered disulfide mutations,
proline substitution mutations, glycine replacement
mutations, and/or cavity filling mutations.

| | | Response Shift with PIV1-8 mAb | | |
|---|---|---|---|---|
| | Protein | No | 53° C. stress | 56° C. stress |
| | expression | stress | resistance | resistance |
| Mutant ID | (mg/L) | (nm) | (nm) | (nm) |
| WT Control | 9.8 | −0.068 | 0.004 | −0.113 |
| Literature prefusion | 44.5 | 0.709 | 0.646 | 0.119 |
| comparison | | | | |
| PIV1047 | 32.5 | 0.790 | 0.862 | 0.754 |
| PIV1048 | 16.4 | 0.737 | 0.783 | 0.710 |
| PIV1049 | 14.0 | 0.636 | 0.883 | 0.294 |
| PIV1050 | 27.8 | 0.848 | 0.907 | 0.695 |
| PIV1051 | 24.9 | 0.725 | 0.744 | 0.322 |
| PIV1052 | 24.9 | 0.670 | −0.026 | −0.094 |
| PIV1053 | 57.7 | 0.857 | 0.901 | 0.776 |
| PIV1054 | 15.8 | 0.764 | 0.826 | 0.723 |
| PIV1055 | 14.0 | 0.757 | 0.787 | 0.682 |
| PIV1056 | 53.2 | 0.739 | 0.805 | 0.751 |
| PIV1057 | 17.1 | 0.718 | 0.808 | 0.737 |
| PIV1058 | 26.5 | 0.688 | 0.704 | 0.543 |
| PIV1059 | 40.5 | 0.841 | 0.903 | 0.784 |
| PIV1060 | 7.8 | 0.584 | 0.671 | 0.597 |
| PIV1061 | 8.1 | 0.619 | 0.648 | 0.526 |
| PIV1062 | 13.7 | 0.619 | 0.714 | 0.623 |
| PIV1063 | 5.9 | −0.116 | −0.036 | −0.069 |
| PIV1064 | 47.9 | 0.912 | 0.897 | 0.702 |
| PIV1065 | 52.1 | 0.809 | 0.816 | 0.677 |
| PIV1066 | 8.1 | 0.642 | 0.722 | 0.582 |
| PIV1067 | 31.8 | 0.849 | 0.896 | 0.728 |
| PIV1068 | 8.4 | 0.628 | 0.650 | 0.559 |

TABLE 41
Protein expression and thermal stability of PIV1 F protein mutants
comprising engineered cleavage site and cavity filling mutations.

| | | Response Shift with PIV1-8 mAb | | |
|---|---|---|---|---|
| | Protein | No | 53° C. stress | 56° C. stress |
| | expression | stress | resistance | resistance |
| Mutant ID | (mg/L) | (nm) | (nm) | (nm) |
| Literature | 61.4 | 0.769 | 0.717 | 0.458 |
| prefusion | | | | |
| comparison | | | | |
| PIV1069 | 57.4 | 0.857 | 0.847 | 0.819 |
| PIV1070 | 51.8 | 0.876 | 0.840 | 0.859 |

### Example 13: Design and Preparation of PIV3 F Protein Mutants

This example illustrates the design and preparation of various PIV3 F protein mutants, which include a fibritin foldon trimerization domain and introduced amino acid mutations, such as engineered disulfide bond mutations, cavity filling mutations, proline substitution mutations, glycine replacement mutations, electrostatic mutations, cleavage site mutations, or a combination thereof. Exemplary PIV3 F protein mutants, each of which is identified by an unique identifier, such as PIV3025, PIV3031, etc., are provided in Tables 42-47. Each of these mutants was designed and prepared based on the amino acid sequence set forth in SEQ ID NO:305. Amino acid residues 1-481 of the sequence of SEQ ID NO:305 are identical to amino acid residues 1-481 of the F0 precursor polypeptide of native PIV3 as set forth in SEQ ID NO:300. Therefore, the amino acid sequences of these exemplary F mutants are identical except for the introduced amino acid mutations as noted for each mutant listed in Tables 42-47. Each of these PIV3 F protein mutants comprises two polypeptide chains. One of the polypeptide chains, the F2 polypeptide, comprises amino acids 19-109 of SEQ ID NO:305 except for the introduced mutations as noted. The other polypeptide chain comprises the F1 polypeptide (residues 110-481) linked to a foldon trimerization domain (residues 486-512) via a GGGS linker (residues 482-485). The signal peptide (residues 1-18) of SEQ ID NO:305 were cleaved from the F0 precursor during the expression process.

TABLE 42
Exemplary PIV3 F Protein Mutants Comprising Engineered Disulfide Mutations

| | | Amino Acid Sequence (residues 110-481 for F1 |
|---|---|---|
| Mutant ID | Mutations | polypeptide and residues 19-109 for F2 polypeptide) |
| PIV3025 | V175C-A202C | F1 (SEQ ID NO: 306): |
| | | FFGGVIGTIALGVATSAQITAAVALVEAKQARSDIEKLKEAI |
| | | RDTNKAVQSVQSSVGNLIVAIKSCQDYVNKEIVPSIARLGC |
| | | EAAGLQLGICLTQHYSELTNIFGDNIGSLQEKGIKLQGIASL |
| | | YRTNITEIFTTSTVDKYDIYDLLFTESIKVRVIDVDLNDYSITL |
| | | QVRLPLLTRLLNTQIYKVDSISYNIQNREWYIPLPSHIMTKG |
| | | AFLGGADVKECIEAFSSYICPSDPGFVLNHEMESCLSGNI |
| | | SQCPRTTVTSDIVPRYAFVNGGVVANCITTTCTCNGIGNRI |
| | | NQPPDQGVKIITHKECNTIGINGMLFNTNKEGTLAFYTPDD |
| | | ITLNNSVALDPIDISIELNKAKSDLEESKEWIRRSNQKL |
| | | F2 (SEQ ID NO: 307): |
| | | QIDITKLQHVGVLVNSPKGMKISQNFETRYLILSLIPKIEDS |
| | | NSCGDQQIKQYKRLLDRLIIPLYDGLRLQKDVIVTNQESNE |
| | | NTDPRTER |
| PIV3031 | S160C-V170C | F1 (SEQ ID NO: 308): |
| | | FFGGVIGTIALGVATSAQITAAVALVEAKQARSDIEKLKEAI |
| | | RDTNKAVQCVQSSVGNLICAIKSVQDYVNKEIVPSIARLGC |
| | | EAAGLQLGIALTQHYSELTNIFGDNIGSLQEKGIKLQGIASL |
| | | YRTNITEIFTTSTVDKYDIYDLLFTESIKVRVIDVDLNDYSITL |
| | | QVRLPLLTRLLNTQIYKVDSISYNIQNREWYIPLPSHIMTKG |
| | | AFLGGADVKECIEAFSSYICPSDPGFVLNHEMESCLSGNI |
| | | SQCPRTTVTSDIVPRYAFVNGGVVANCITTTCTCNGIGNRI |
| | | NQPPDQGVKIITHKECNTIGINGMLFNTNKEGTLAFYTPDD |
| | | ITLNNSVALDPIDISIELNKAKSDLEESKEWIRRSNQKL |
| | | F2 (SEQ ID NO: 309): |
| | | QIDITKLQHVGVLVNSPKGMKISQNFETRYLILSLIPKIEDS |
| | | NSCGDQQIKQYKRLLDRLIIPLYDGLRLQKDVIVTNQESNE |
| | | NTDPRTER |

TABLE 43
Exemplary PIV3 F Protein Mutants Comprising Proline Substitution Mutations

| | | Amino Acid Sequence (residues 110-481 for F1 |
|---|---|---|
| Mutant ID | Mutations | polypeptide and residues 19-109 for F2 polypeptide) |
| PIV3033 | S164P | F1 (SEQ ID NO: 310): |
| | | FFGGVIGTIALGVATSAQITAAVALVEAKQARSDIEKLKEAI |
| | | RDTNKAVQSVQSPVGNLIVAIKSVQDYVNKEIVPSIARLGC |
| | | EAAGLQLGIALTQHYSELTNIFGDNIGSLQEKGIKLQGIASL |
| | | YRTNITEIFTTSTVDKYDIYDLLFTESIKVRVIDVDLNDYSITL |
| | | QVRLPLLTRLLNTQIYKVDSISYNIQNREWYIPLPSHIMTKG |
| | | AFLGGADVKECIEAFSSYICPSDPGFVLNHEMESCLSGNI |
| | | SQCPRTTVTSDIVPRYAFVNGGVVANCITTTCTCNGIGNRI |
| | | NQPPDQGVKIITHKECNTIGINGMLFNTNKEGTLAFYTPDD |
| | | ITLNNSVALDPIDISIELNKAKSDLEESKEWIRRSNQKL |
| | | F2 (SEQ ID NO: 311): |
| | | QIDITKLQHVGVLVNSPKGMKISQNFETRYLILSLIPKIEDS |
| | | NSCGDQQIKQYKRLLDRLIIPLYDGLRLQKDVIVTNQESNE |
| | | NTDPRTER |

TABLE 44
Exemplary PIV3 F Protein Mutants Comprising Glycine Replacement Mutations,
Electrostatic Mutations and/or Cavity Filing Mutations

| | | Amino Acid Sequence (residues 110-481 for F1 |
|---|---|---|
| Mutant ID | Mutations | polypeptide and residues 19-109 for F2 polypeptide) |
| PIV3037 | G196A | F1 (SEQ ID NO: 312): |
| | | FFGGVIGTIALGVATSAQITAAVALVEAKQARSDIEKLKEAI |
| | | RDTNKAVQSVQSSVGNLIVAIKSVQDYVNKEIVPSIARLGC |
| | | EAAALQLGIALTQHYSELTNIFGDNIGSLQEKGIKLQGIASL |
| | | YRTNITEIFTTSTVDKYDIYDLLFTESIKVRVIDVDLNDYSITL |
| | | QVRLPLLTRLLNTQIYKVDSISYNIQNREWYIPLPSHIMTKG |
| | | AFLGGADVKECIEAFSSYICPSDPGFVLNHEMESCLSGNI |
| | | SQCPRTTVTSDIVPRYAFVNGGVVANCITTTCTCNGIGNRI |
| | | NQPPDQGVKIITHKECNTIGINGMLFNTNKEGTLAFYTPDD |
| | | ITLNNSVALDPIDISIELNKAKSDLEESKEWIRRSNQKL |
| | | F2 (SEQ ID NO: 313): |
| | | QIDITKLQHVGVLVNSPKGMKISQNFETRYLILSLIPKIEDS |
| | | NSCGDQQIKQYKRLLDRLIIPLYDGLRLQKDVIVTNQESNE |
| | | NTDPRTER |
| PIV3039 | G219P | F1 (SEQ ID NO: 314): |
| | | FFGGVIGTIALGVATSAQITAAVALVEAKQARSDIEKLKEAI |
| | | RDTNKAVQSVQSSVGNLIVAIKSVQDYVNKEIVPSIARLGC |
| | | EAAGLQLGIALTQHYSELTNIFGDNIPSLQEKGIKLQGIASL |
| | | YRTNITEIFTTSTVDKYDIYDLLFTESIKVRVIDVDLNDYSITL |
| | | QVRLPLLTRLLNTQIYKVDSISYNIQNREWYIPLPSHIMTKG |
| | | AFLGGADVKECIEAFSSYICPSDPGFVLNHEMESCLSGNI |
| | | SQCPRTTVTSDIVPRYAFVNGGWVANCITTTCTCNGIGNRI |
| | | NQPPDQGVKIITHKECNTIGINGMLFNTNKEGTLAFYTPDD |
| | | ITLNNSVALDPIDISIELNKAKSDLEESKEWIRRSNQKL |
| | | F2 (SEQ ID NO: 315): |
| | | QIDITKLQHVGVLVNSPKGMKISQNFETRYLILSLIPKIEDS |
| | | NSCGDQQIKQYKRLLDRLIIPLYDGLRLQKDVIVTNQESNE |
| | | NTDPRTER |
| PIV3041 | G230A | F1 (SEQ ID NO: 316): |
| | | FFGGVIGTIALGVATSAQITAAVALVEAKQARSDIEKLKEAI |
| | | RDTNKAVQSVQSSVGNLIVAIKSVQDYVNKEIVPSIARLGC |
| | | EAAGLQLGIALTQHYSELTNIFGDNIGSLQEKGIKLQAIASL |
| | | YRTNITEIFTTSTVDKYDIYDLLFTESIKVRVIDVDLNDYSITL |
| | | QVRLPLLTRLLNTQIYKVDSISYNIQNREWYIPLPSHIMTKG |
| | | AFLGGADVKECIEAFSSYICPSDPGFVLNHEMESCLSGNI |
| | | SQCPRTTVTSDIVPRYAFVNGGVVANCITTTCTCNGIGNRI |
| | | NQPPDQGVKIITHKECNTIGINGMLFNTNKEGTLAFYTPDD |
| | | ITLNNSVALDPIDISIELNKAKSDLEESKEWIRRSNQKL |
| | | F2 (SEQ ID NO: 317): |
| | | QIDITKLQHVGVLVNSPKGMKISQNFETRYLILSLIPKIEDSN |
| | | SCGDQQIKQYKRLLDRLIIPLYDGLRLQKDVIVTNQESNEN |
| | | TDPRTER |
| PIV3045 | E182L | F1 (SEQ ID NO: 318): |
| | | FFGGVIGTIALGVATSAQITAAVALVEAKQARSDIEKLKEAI |
| | | RDTNKAVQSVQSSVGNLIVAIKSVQDYVNKLIVPSIARLGC |
| | | EAAGLQLGIALTQHYSELTNIFGDNIGSLQEKGIKLQGIASL |
| | | YRTNITEIFTTSTVDKYDIYDLLFTESIKVRVIDVDLNDYSITL |
| | | QVRLPLLTRLLNTQIYKVDSISYNIQNREWYIPLPSHIMTKG |
| | | AFLGGADVKECIEAFSSYICPSDPGFVLNHEMESCLSGNI |
| | | SQCPRTTVTSDIVPRYAFVNGGWVANCITTTCTCNGIGNRI |
| | | NQPPDQGVKIITHKECNTIGINGMLFNTNKEGTLAFYTPDD |
| | | ITLNNSVALDPIDISIELNKAKSDLEESKEWIRRSNQKL |
| | | F2 (SEQ ID NO: 319): |
| | | QIDITKLQHVGVLVNSPKGMKISQNFETRYLILSLIPKIEDS |
| | | NSCGDQQIKQYKRLLDRLIIPLYDGLRLQKDVIVTNQESNE |
| | | NTDPRTER |
| PIV3051 | S470A | F1 (SEQ ID NO: 320): |
| | | FFGGVIGTIALGVATSAQITAAVALVEAKQARSDIEKLKEAI |
| | | RDTNKAVQSVQSSVGNLIVAIKSVQDYVNKEIVPSIARLGC |
| | | EAAGLQLGIALTQHYSELTNIFGDNIGSLQEKGIKLQGIASL |
| | | YRTNITEIFTTSTVDKYDIYDLLFTESIKVRVIDVDLNDYSITL |
| | | QVRLPLLTRLLNTQIYKVDSISYNIQNREWYIPLPSHIMTKG |
| | | AFLGGADVKECIEAFSSYICPSDPGFVLNHEMESCLSGNI |
| | | SQCPRTTVTSDIVPRYAFVNGGVVANCITTTCTCNGIGNRI |
| | | NQPPDQGVKIITHKECNTIGINGMLFNTNKEGTLAFYTPDD |
| | | ITLNNSVALDPIDISIELNKAKSDLEEAKEWIRRSNQKL |
| | | F2 (SEQ ID NO: 321): |
| | | QIDITKLQHVGVL VNSPKGMKISQNFETRYLILSLIPKIEDSN |
| | | SCGDQQIKQYKRLLDRLIIPLYDGLRLQKDVIVTNQESNEN |
| | | TDPRTER |
| PIV3052 | S477A | F1 (SEQ ID NO: 322): |
| | | FFGGVIGTIALGVATSAQITAAVALVEAKQARSDIEKLKEAI |
| | | RDTNKAVQSVQSSVGNLIVAIKSVQDYVNKEIVPSIARLGC |
| | | EAAGLQLGIALTQHYSELTNIFGDNIGSLQEKGIKLQGIASL |
| | | YRTNITEIFTTSTVDKYDIYDLLFTESIKVRVIDVDLNDYSITL |
| | | QVRLPLLTRLLNTQIYKVDSISYNIQNREWYIPLPSHIMTKG |
| | | AFLGGADVKECIEAFSSYICPSDPGFVLNHEMESCLSGNI |
| | | SQCPRTTVTSDIVPRYAFVNGGWVANCITTTCTCNGIGNRI |
| | | NQPPDQGVKIITHKECNTIGINGMLFNTNKEGTLAFYTPDD |
| | | ITLNNSVALDPIDISIELNKAKSDLEESKEWIRRANQKL |
| | | F2 (SEQ ID NO: 323): |
| | | QIDITKLQHVGVLVNSPKGMKISQNFETRYLILSLIPKIEDS |
| | | NSCGDQQIKQYKRLLDRLIIPLYDGLRLQKDVIVTNQESNE |
| | | NTDPRTER |
| PIV3053 | S470A, S477A | F1 (SEQ ID NO: 324): |
| | | FFGGVIGTIALGVATSAQITAAVALVEAKQARSDIEKLKEAI |
| | | RDTNKAVQSVQSSVGNLIVAIKSVQDYVNKEIVPSIARLGC |
| | | EAAGLQLGIALTQHYSELTNIFGDNIGSLQEKGIKLQGIASL |
| | | YRTNITEIFTTSTVDKYDIYDLLFTESIKVRVIDVDLNDYSITL |
| | | QVRLPLLTRLLNTQIYKVDSISYNIQNREWYIPLPSHIMTKG |
| | | AFLGGADVKECIEAFSSYICPSDPGFVLNHEMESCLSGNI |
| | | SQCPRTTVTSDIVPRYAFVNGGVVANCITTTCTCNGIGNRI |
| | | NQPPDQGVKIITHKECNTIGINGMLFNTNKEGTLAFYTPDD |
| | | ITLNNSVALDPIDISIELNKAKSDLEEAKEWIRRANQKL |
| | | F2 (SEQ ID NO: 325): |
| | | QIDITKLQHVGVLVNSPKGMKISQNFETRYLILSLIPKIEDSN |
| | | SCGDQQIKQYKRLLDRLIIPLYDGLRLQKDVIVTNQESNEN |
| | | TDPRTER |
| PIV3107 | A463L | F1 (SEQ ID NO: 326): |
| | | FFGGVIGTIALGVATSAQITAAVALVEAKQARSDIEKLKEAI |
| | | RDTNKAVQSVQSSVGNLIVAIKSVQDYVNKEIVPSIARLGC |
| | | EAAGLQLGIALTQHYSELTNIFGDNIGSLQEKGIKLQGIASL |
| | | YRTNITEIFTTSTVDKYDIYDLLFTESIKVRVIDVDLNDYSITL |
| | | QVRLPLLTRLLNTQIYKVDSISYNIQNREWYIPLPSHIMTKG |
| | | AFLGGADVKECIEAFSSYICPSDPGFVLNHEMESCLSGNI |
| | | SQCPRTTVTSDIVPRYAFVNGGWVANCITTTCTCNGIGNRI |
| | | NQPPDQGVKIITHKECNTIGINGMLFNTNKEGTLAFYTPDD |
| | | ITLNNSVALDPIDISIELNKLKSDLEESKEWIRRSNQKL |
| | | F2 (SEQ ID NO: 327): |
| | | QIDITKLQHVGVLVNSPKGMKISQNFETRYLILSLIPKIEDS |
| | | NSCGDQQIKQYKRLLDRLIIPLYDGLRLQKDVIVTNQESNE |
| | | NTDPRTER |
| PIV3110 | S470A, | F1 (SEQ ID NO: 328): |
| | S477A, G230A | FFGGVIGTIALGVATSAQITAAVALVEAKQARSDIEKLKEAI |
| | | RDTNKAVQSVQSSVGNLIVAIKSVQDYVNKEIVPSIARLGC |
| | | EAAGLQLGIALTQHYSELTNIFGDNIGSLQEKGIKLQAIASL |
| | | YRTNITEIFTTSTVDKYDIYDLLFTESIKVRVIDVDLNDYSITL |
| | | QVRLPLLTRLLNTQIYKVDSISYNIQNREWYIPLPSHIMTKG |
| | | AFLGGADVKECIEAFSSYICPSDPGFVLNHEMESCLSGNI |
| | | SQCPRTTVTSDIVPRYAFVNGGVVANCITTTCTCNGIGNRI |
| | | NQPPDQGVKIITHKECNTIGINGMLFNTNKEGTLAFYTPDD |
| | | ITLNNSVALDPIDISIELNKAKSDLEEAKEWIRRANQKL |
| | | F2 (SEQ ID NO: 329): |
| | | QIDITKLQHVGVLVNSPKGMKISQNFETRYLILSLIPKIEDS |
| | | NSCGDQQIKQYKRLLDRLIIPLYDGLRLQKDVIVTNQESNE |
| | | NTDPRTER |
| PIV3111 | S470A, | F1 (SEQ ID NO: 330): |
| | S477A, A463L | FFGGVIGTIALGVATSAQITAAVALVEAKQARSDIEKLKEAI |
| | | RDTNKAVQSVQSSVGNLIVAIKSVQDYVNKEIVPSIARLGC |
| | | EAAGLQLGIALTQHYSELTNIFGDNIGSLQEKGIKLQGIASL |
| | | YRTNITEIFTTSTVDKYDIYDLLFTESIKVRVIDVDLNDYSITL |
| | | QVRLPLLTRLLNTQIYKVDSISYNIQNREWYIPLPSHIMTKG |
| | | AFLGGADVKECIEAFSSYICPSDPGFVLNHEMESCLSGNI |
| | | SQCPRTTVTSDIVPRYAFVNGGVVANCITTTCTCNGIGNRI |
| | | NQPPDQGVKIITHKECNTIGINGMLFNTNKEGTLAFYTPDD |
| | | ITLNNSVALDPIDISIELNKLKSDLEEAKEWIRRANQKL |
| | | F2 (SEQ ID NO: 331): |
| | | QIDITKLQHVGVLVNSPKGMKISQNFETRYLILSLIPKIEDS |
| | | NSCGDQQIKQYKRLLDRLIIPLYDGLRLQKDVIVTNQESNE |
| | | NTDPRTER |

TABLE 45
Exemplary PIV3 F Protein Mutants Comprising Electrostatic Mutations

| | | Amino Acid Sequence (residues 110-481 for F1 |
|---|---|---|
| Mutant ID | Mutations | polypeptide and residues 19-109 for F2 polypeptide) |
| PIV3060 | D455S | F1 (SEQ ID NO: 332): |
| | | FFGGVIGTIALGVATSAQITAAVALVEAKQARSDIEKLKEAI |
| | | RDTNKAVQSVQSSVGNLIVAIKSVQDYVNKEIVPSIARLGC |
| | | EAAGLQLGIALTQHYSELTNIFGDNIGSLQEKGIKLQGIASL |
| | | YRTNITEIFTTSTVDKYDIYDLLFTESIKVRVIDVDLNDYSITL |
| | | QVRLPLLTRLLNTQIYKVDSISYNIQNREWYIPLPSHIMTKG |
| | | AFLGGADVKECIEAFSSYICPSDPGFVLNHEMESCLSGNI |
| | | SQCPRTTVTSDIVPRYAFVNGGWVANCITTTCTCNGIGNRI |
| | | NQPPDQGVKIITHKECNTIGINGMLFNTNKEGTLAFYTPDD |
| | | ITLNNSVALDPISISIELNKAKSDLEESKEWIRRSNQKL |
| | | F2 (SEQ ID NO: 333): |
| | | QIDITKLQHVGVLVNSPKGMKISQNFETRYLILSLIPKIEDS |
| | | NSCGDQQIKQYKRLLDRLIIPLYDGLRLQKDVIVTNQESNE |
| | | NTDPRTER |

TABLE 46
Exemplary PIV3 F Protein Mutants Comprising Cleavage Site Mutations

| | | Amino Acid Sequence (residues 110-481 for F1 |
|---|---|---|
| Mutant ID | Mutations | polypeptide and residues 19-109 for F2 polypeptide) |
| PIV3014 | RTER(106- | F1 (SEQ ID NO: 334): |
| | 109)→GSAS | FFGGVIGTIALGVATSAQITAAVALVEAKQARSDIEKLKEAI |
| | | RDTNKAVQSVQSSVGNLIVAIKSVQDYVNKEIVPSIARLGC |
| | | EAAGLQLGIALTQHYSELTNIFGDNIGSLQEKGIKLQGIASL |
| | | YRTNITEIFTTSTVDKYDIYDLLFTESIKVRVIDVDLNDYSITL |
| | | QVRLPLLTRLLNTQIYKVDSISYNIQNREWYIPLPSHIMTKG |
| | | AFLGGADVKECIEAFSSYICPSDPGFVLNHEMESCLSGNI |
| | | SQCPRTTVTSDIVPRYAFVNGGVVANCITTTCTCNGIGNRI |
| | | NQPPDQGVKIITHKECNTIGINGMLFNTNKEGTLAFYTPDD |
| | | ITLNNSVALDPIDISIELNKAKSDLEESKEWIRRSNQKL |
| | | F2 (SEQ ID NO: 335): |
| | | QIDITKLQHVGVLVNSPKGMKISQNFETRYLILSLIPKIEDS |
| | | NSCGDQQIKQYKRLLDRLIIPLYDGLRLQKDVIVTNQESNE |
| | | NTDPGSAS |

TABLE 47
Exemplary PIV3 F Protein Mutants Comprising Engineered Disulfide Mutations and
Cavity Filling Mutations

| | | Amino Acid Sequence (residues 110-481 for F1 |
|---|---|---|
| Mutant ID | Mutations | polypeptide and residues 19-109 for F2 polypeptide) |
| PIV3108 | Q162C-L168C, | F1 (SEQ ID NO: 336): |
| | S470A, S477A | FFGGVIGTIALGVATSAQITAAVALVEAKQARSDIEKLKEAI |
| | | RDTNKAVQSVCSSVGNCIVAIKSVQDYVNKEIVPSIARLGC |
| | | EAAGLQLGIALTQHYSELTNIFGDNIGSLQEKGIKLQGIASL |
| | | YRTNITEIFTTSTVDKYDIYDLLFTESIKVRVIDVDLNDYSITL |
| | | QVRLPLLTRLLNTQIYKVDSISYNIQNREWYIPLPSHIMTKG |
| | | AFLGGADVKECIEAFSSYICPSDPGFVLNHEMESCLSGNI |
| | | SQCPRTTVTSDIVPRYAFVNGGVVANCITTTCTCNGIGNRI |
| | | NQPPDQGVKIITHKECNTIGINGMLFNTNKEGTLAFYTPDD |
| | | ITLNNSVALDPIDISIELNKAKSDLEEAKEWIRRANQKL |
| | | F2 (SEQ ID NO: 337): |
| | | QIDITKLQHVGVLVNSPKGMKISQNFETRYLILSLIPKIEDS |
| | | NSCGDQQIKQYKRLLDRLIIPLYDGLRLQKDVIVTNQESNE |
| | | NTDPRTER |
| PIV3109 | S160C-V170C, | F1 (SEQ ID NO: 338): |
| | S470A, S477A | FFGGVIGTIALGVATSAQITAAVALVEAKQARSDIEKLKEAI |
| | | RDTNKAVQCVQSSVGNLICAIKSVQDYVNKEIVPSIARLGC |
| | | EAAGLQLGIALTQHYSELTNIFGDNIGSLQEKGIKLQGIASL |
| | | YRTNITEIFTTSTVDKYDIYDLLFTESIKVRVIDVDLNDYSITL |
| | | QVRLPLLTRLLNTQIYKVDSISYNIQNREWYIPLPSHIMTKG |
| | | AFLGGADVKECIEAFSSYICPSDPGFVLNHEMESCLSGNI |
| | | SQCPRTTVTSDIVPRYAFVNGGVVANCITTTCTCNGIGNRI |
| | | NQPPDQGVKIITHKECNTIGINGMLFNTNKEGTLAFYTPDD |
| | | ITLNNSVALDPIDISIELNKAKSDLEEAKEWIRRANQKL |
| | | F2 (SEQ ID NO: 339): |
| | | QIDITKLQHVGVLVNSPKGMKISQNFETRYLILSLIPKIEDS |
| | | NSCGDQQIKQYKRLLDRLIIPLYDGLRLQKDVIVTNQESNE |
| | | NTDPRTER |
| PIV3112 | Q162C-L168C, | F1 (SEQ ID NO: 340): |
| | A463V, 1474Y, | FFGGVIGTIALGVATSAQITAAVALVEAKQARSDIEKLKEAI |
| | G230A | RDTNKAVQSVCSSVGNCIVAIKSVQDYVNKEIVPSIARLGC |
| | | EAAGLQLGIALTQHYSELTNIFGDNIGSLQEKGIKLQAIASL |
| | | YRTNITEIFTTSTVDKYDIYDLLFTESIKVRVIDVDLNDYSITL |
| | | QVRLPLLTRLLNTQIYKVDSISYNIQNREWYIPLPSHIMTKG |
| | | AFLGGADVKECIEAFSSYICPSDPGFVLNHEMESCLSGNI |
| | | SQCPRTTVTSDIVPRYAFVNGGWVANCITTTCTCNGIGNRI |
| | | NQPPDQGVKIITHKECNTIGINGMLFNTNKEGTLAFYTPDD |
| | | ITLNNSVALDPIDISIELNKVKSDLEESKEWYRRSNQKL |
| | | F2 (SEQ ID NO: 341): |
| | | QIDITKLQHVGVLVNSPKGMKISQNFETRYLILSLIPKIEDS |
| | | NSCGDQQIKQYKRLLDRLIIPLYDGLRLQKDVIVTNQESNE |
| | | NTDPRTER |
| PIV3113 | Q162C-L168C, | F1 (SEQ ID NO: 342): |
| | S470A, | FFGGVIGTIALGVATSAQITAAVALVEAKQARSDIEKLKEAI |
| | S477A, G230A | RDTNKAVQSVCSSVGNCIVAIKSVQDYVNKEIVPSIARLGC |
| | | EAAGLQLGIALTQHYSELTNIFGDNIGSLQEKGIKLQAIASL |
| | | YRTNITEIFTTSTVDKYDIYDLLFTESIKVRVIDVDLNDYSITL |
| | | QVRLPLLTRLLNTQIYKVDSISYNIQNREWYIPLPSHIMTKG |
| | | AFLGGADVKECIEAFSSYICPSDPGFVLNHEMESCLSGNI |
| | | SQCPRTTVTSDIVPRYAFVNGGVVANCITTTCTCNGIGNRI |
| | | NQPPDQGVKIITHKECNTIGINGMLFNTNKEGTLAFYTPDD |
| | | ITLNNSVALDPIDISIELNKAKSDLEEAKEWIRRANQKL |
| | | F2 (SEQ ID NO: 343): |
| | | QIDITKLQHVGVLVNSPKGMKISQNFETRYLILSLIPKIEDS |
| | | NSCGDQQIKQYKRLLDRLIIPLYDGLRLQKDVIVTNQESNE |
| | | NTDPRTER |
| PIV3114 | Q162C-L168C, | F1 (SEQ ID NO: 344): |
| | G230A, A463L | FFGGVIGTIALGVATSAQITAAVALVEAKQARSDIEKLKEAI |
| | | RDTNKAVQSVCSSVGNCIVAIKSVQDYVNKEIVPSIARLGC |
| | | EAAGLQLGIALTQHYSELTNIFGDNIGSLQEKGIKLQAIASL |
| | | YRTNITEIFTTSTVDKYDIYDLLFTESIKVRVIDVDLNDYSITL |
| | | QVRLPLLTRLLNTQIYKVDSISYNIQNREWYIPLPSHIMTKG |
| | | AFLGGADVKECIEAFSSYICPSDPGFVLNHEMESCLSGNI |
| | | SQCPRTTVTSDIVPRYAFVNGGVVANCITTTCTCNGIGNRI |
| | | NQPPDQGVKIITHKECNTIGINGMLFNTNKEGTLAFYTPDD |
| | | ITLNNSVALDPIDISIELNKLKSDLEESKEWIRRSNQKL |
| | | F2 (SEQ ID NO: 345): |
| | | QIDITKLQHVGVLVNSPKGMKISQNFETRYLILSLIPKIEDS |
| | | NSCGDQQIKQYKRLLDRLIIPLYDGLRLQKDVIVTNQESNE |
| | | NTDPRTER |
| PIV3115 | Q162C-L168C, | F1 (SEQ ID NO: 346): |
| | S470A, | FFGGVIGTIALGVATSAQITAAVALVEAKQARSDIEKLKEAI |
| | S477A, A463L, | RDTNKAVQSVCSSVGNCIVAIKSVQDYVNKEIVPSIARLGC |
| | G230A | EAAGLQLGIALTQHYSELTNIFGDNIGSLQEKGIKLQAIASL |
| | | YRTNITEIFTTSTVDKYDIYDLLFTESIKVRVIDVDLNDYSITL |
| | | QVRLPLLTRLLNTQIYKVDSISYNIQNREWYIPLPSHIMTKG |
| | | AFLGGADVKECIEAFSSYICPSDPGFVLNHEMESCLSGNI |
| | | SQCPRTTVTSDIVPRYAFVNGGWVANCITTTCTCNGIGNRI |
| | | NQPPDQGVKIITHKECNTIGINGMLFNTNKEGTLAFYTPDD |
| | | ITLNNSVALDPIDISIELNKLKSDLEEAKEWIRRANQKL |
| | | F2 (SEQ ID NO: 347): |
| | | QIDITKLQHVGVLVNSPKGMKISQNFETRYLILSLIPKIEDS |
| | | NSCGDQQIKQYKRLLDRLIIPLYDGLRLQKDVIVTNQESNE |
| | | NTDPRTER |
| PIV3116 | S160C-V170C, | F1 (SEQ ID NO: 348): |
| | A463V, 1474Y, | FFGGVIGTIALGVATSAQITAAVALVEAKQARSDIEKLKEAI |
| | G230A | RDTNKAVQCVQSSVGNLICAIKSVQDYVNKEIVPSIARLGC |
| | | EAAGLQLGIALTQHYSELTNIFGDNIGSLQEKGIKLQAIASL |
| | | YRTNITEIFTTSTVDKYDIYDLLFTESIKVRVIDVDLNDYSITL |
| | | QVRLPLLTRLLNTQIYKVDSISYNIQNREWYIPLPSHIMTKG |
| | | AFLGGADVKECIEAFSSYICPSDPGFVLNHEMESCLSGNI |
| | | SQCPRTTVTSDIVPRYAFVNGGVVANCITTTCTONGIGNRI |
| | | NQPPDQGVKIITHKECNTIGINGMLFNTNKEGTLAFYTPDD |
| | | ITLNNSVALDPIDISIELNKVKSDLEESKEWYRRSNQKL |
| | | F2 (SEQ ID NO: 349): |
| | | QIDITKLQHVGVLVNSPKGMKISQNFETRYLILSLIPKIEDS |
| | | NSCGDQQIKQYKRLLDRLIIPLYDGLRLQKDVIVTNQESNE |
| | | NTDPRTER |
| PIV3117 | S160C-V170C, | F1 (SEQ ID NO: 350): |
| | S470A, | FFGGVIGTIALGVATSAQITAAVALVEAKQARSDIEKLKEAI |
| | S477A, G230A | RDTNKAVQCVQSSVGNLICAIKSVQDYVNKEIVPSIARLGC |
| | | EAAGLQLGIALTQHYSELTNIFGDNIGSLQEKGIKLQAIASL |
| | | YRTNITEIFTTSTVDKYDIYDLLFTESIKVRVIDVDLNDYSITL |
| | | QVRLPLLTRLLNTQIYKVDSISYNIQNREWYIPLPSHIMTKG |
| | | AFLGGADVKECIEAFSSYICPSDPGFVLNHEMESCLSGNI |
| | | SQCPRTTVTSDIVPRYAFVNGGWVANCITTTCTCNGIGNRI |
| | | NQPPDQGVKIITHKECNTIGINGMLFNTNKEGTLAFYTPDD |
| | | ITLNNSVALDPIDISIELNKAKSDLEEAKEWIRRANQKL |
| | | F2 (SEQ ID NO: 351): |
| | | QIDITKLQHVGVLVNSPKGMKISQNFETRYLILSLIPKIEDS |
| | | NSCGDQQIKQYKRLLDRLIIPLYDGLRLQKDVIVTNQESNE |
| | | NTDPRTER |
| PIV3118 | S160C-V170C, | F1 (SEQ ID NO: 352): |
| | A463L, G230A | FFGGVIGTIALGVATSAQITAAVALVEAKQARSDIEKLKEAI |
| | | RDTNKAVQCVQSSVGNLICAIKSVQDYVNKEIVPSIARLGC |
| | | EAAGLQLGIALTQHYSELTNIFGDNIGSLQEKGIKLQAIASL |
| | | YRTNITEIFTTSTVDKYDIYDLLFTESIKVRVIDVDLNDYSITL |
| | | QVRLPLLTRLLNTQIYKVDSISYNIQNREWYIPLPSHIMTKG |
| | | AFLGGADVKECIEAFSSYICPSDPGFVLNHEMESCLSGNI |
| | | SQCPRTTVTSDIVPRYAFVNGGVVANCITTTCTCNGIGNRI |
| | | NQPPDQGVKIITHKECNTIGINGMLFNTNKEGTLAFYTPDD |
| | | ITLNNSVALDPIDISIELNKLKSDLEESKEWIRRSNQKL |
| | | F2 (SEQ ID NO: 353): |
| | | QIDITKLQHVGVLVNSPKGMKISQNFETRYLILSLIPKIEDS |
| | | NSCGDQQIKQYKRLLDRLIIPLYDGLRLQKDVIVTNQESNE |
| | | NTDPRTER |
| PIV3119 | S160C-V170C, | F1 (SEQ ID NO: 354): |
| | S470A, | FFGGVIGTIALGVATSAQITAAVALVEAKQARSDIEKLKEAI |
| | S477A, A463L, | RDTNKAVQCVQSSVGNLICAIKSVQDYVNKEIVPSIARLGC |
| | G230A | EAAGLQLGIALTQHYSELTNIFGDNIGSLQEKGIKLQAIASL |
| | | YRTNITEIFTTSTVDKYDIYDLLFTESIKVRVIDVDLNDYSITL |
| | | QVRLPLLTRLLNTQIYKVDSISYNIQNREWYIPLPSHIMTKG |
| | | AFLGGADVKECIEAFSSYICPSDPGFVLNHEMESCLSGNI |
| | | SQCPRTTVTSDIVPRYAFVNGGWVANCITTTCTCNGIGNRI |
| | | NQPPDQGVKIITHKECNTIGINGMLFNTNKEGTLAFYTPDD |
| | | ITLNNSVALDPIDISIELNKLKSDLEEAKEWIRRANQKL |
| | | F2 (SEQ ID NO: 355): |
| | | QIDITKLQHVGVLVNSPKGMKISQNFETRYLILSLIPKIEDS |
| | | NSCGDQQIKQYKRLLDRLIIPLYDGLRLQKDVIVTNQESNE |
| | | NTDPRTER |
| PIV3120 | S160C-V170C, | F1 (SEQ ID NO: 356): |
| | A463L | FFGGVIGTIALGVATSAQITAAVALVEAKQARSDIEKLKEAI |
| | | RDTNKAVQCVQSSVGNLICAIKSVQDYVNKEIVPSIARLGC |
| | | EAAGLQLGIALTQHYSELTNIFGDNIGSLQEKGIKLQGIASL |
| | | YRTNITEIFTTSTVDKYDIYDLLFTESIKVRVIDVDLNDYSITL |
| | | QVRLPLLTRLLNTQIYKVDSISYNIQNREWYIPLPSHIMTKG |
| | | AFLGGADVKECIEAFSSYICPSDPGFVLNHEMESCLSGNI |
| | | SQCPRTTVTSDIVPRYAFVNGGVVANCITTTCTCNGIGNRI |
| | | NQPPDQGVKIITHKECNTIGINGMLFNTNKEGTLAFYTPDD |
| | | ITLNNSVALDPIDISIELNKLKSDLEESKEWIRRSNQKL |
| | | F2 (SEQ ID NO: 357): |
| | | QIDITKLQHVGVLVNSPKGMKISQNFETRYLILSLIPKIEDS |
| | | NSCGDQQIKQYKRLLDRLIIPLYDGLRLQKDVIVTNQESNE |
| | | NTDPRTER |

### Example 14. PIV3 F Protein Mutant Expression Vector Construction

A nucleic acid molecule encoding the consensus PIV3 F0 polypeptide set forth in SEQ ID NO: 305 was mutated using standard molecular biology techniques to encode a precursor polypeptide for a PIV3 F mutant having the introduced amino acid mutations disclosed in example 13. The structure and components of the precursor polypeptide are set forth in FIG. 1 and SEQ ID NO: 305. The precursor polypeptide comprises of a signal peptide (residues 1-18), F2 polypeptide (residues 19-109), F1 polypeptide (residues 110-481), T4 fibritin foldon (residues 486-512), PreScission cleavage site (residues 516-523), Strep Tag II (residues 527-534), and linker sequences (residues 482-485, 513-515, and 524-526).

The protein sequence of SEQ ID NO: 305 was submitted for mammalian codon optimization and synthesis by Genscript (Piscataway, NJ). The synthesized nucleotide sequence was introduced into a commercially available expression vector, pcDNA3.1/Zeo(+) (ThermoFisher Scientific, Waltham, MA) that has been modified to encode the CAG promoter (Yamamura et al., Gene, 108(2), 193-199, 1991) in place of the CMV promoter. Mutagenic oligonucleotides were designed manually and all oligonucleotides, eBlock gene fragments, and gBlock gene fragments were purchased from Integrated DNA Technologies (Coralville, IA). Gene fragments of the mutagenized F allele not synthesized as eBlock or gBlock fragments were generated and amplified by polymerase chain reaction (PCR) with Phusion Flash High-Fidelity PCR Master Mix (ThermoFisher Scientific). Following purification of the linearized expression vector digested using BamHI and Notl, gene fragments of the mutagenized F allele were inserted into the expression vector with NEBuilder HiFi DNA Assembly Kit (New England Biolabs, Ipswich, MA). The presence of the intended sequence was confirmed by DNA sequencing. Plasmid DNA for transfection into ExpiCHO cells was purified with the Qiagen Plasmid Plus Midi Kit (Qiagen, Valencia, CA). For all commercial kits or reagents, procedures were performed according to the manufacturer's protocol.

### Example 15. Transfection of PIV3 F Protein Mutants

Protein for PIV3 F protein mutant evaluation was produced by transient transfection of ExpiCHO cells (ThermoFisher Scientific) with DNA plasmids assembled and prepared as described in Example 14. Transient transfections were carried out according to the manufacturer's protocol. On day 5 post transfection, the cultures were centrifuged, and supernatants were separated from cell pellets. The crude cell supernatants were used for in vitro assays described herein.

### Example 16. Expression and Conformational Integrity of PIV3 F Protein Mutants

The OCTET HTX (Sartorius, Göttingen, Germany) instrument was used to evaluate the expression and conformational integrity for each mutant. All measurements were conducted at 30° C. temperature in 96-well black plates (Corning, Corning, NY) with a final volume of 240 μL per well at a constant agitation rate of 1000 RPM.

### 16A: Quantitation of Expression of PIV3 F Protein Mutants

Crude cell supernatant was used to quantitate the expression levels of PIV3 F protein mutants. Anti-Murine IgG Quantitation (AMQ) Biosensors (Sartorius) were first equilibrated in PB before being dipped into more PB to establish the initial baseline. Biosensors were incubated with a mouse Strep-Tag® II monoclonal antibody (mAb) (Novagen, EMD Millipore Corporation, Temecula, CA) before being equilibrated in PB to establish the experimental baseline. The mAb-bound biosensors were then dipped into crude cell culture supernatant for 2.5 minutes. OCTET data analysis software (version 12.0, Sartorius) was used to generate a standard curve from a serially diluted purified protein reference within the same assay. Titers for protein mutants were then determined based on the standard curve.

The highest expressing amino acid substitutions identified from the screens of the individual engineered disulfide mutants, proline mutations, cavity filling mutants, and electrostatic mutations were combined into the combination mutants. The protein expression of combination mutants was measured using the same procedure as described above. The results are presented in Table 48.

TABLE 48
Protein expression and thermal stability of PIV3 protein mutants.

| | | Response Shift with PIA174 mAb | | |
|---|---|---|---|---|
| | Protein | No | 50° C. stress | 53° C. stress |
| | expression | stress | resistance | resistance |
| Mutant ID | (mg/L) | (nm) | (nm) | (nm) |
| WT Control | 2.9 | 0.600 | ND | ND |
| PIV3014 | 3.3 | 0.654 | 0.507 | 0.440 |
| PIV3025 | 4.9 | −0.001 | −0.014 | −0.054 |
| PIV3031 | 22.0 | 0.812 | 0.333 | 0.273 |
| PIV3033 | 3.0 | 0.651 | 0.332 | 0.296 |
| PIV3037 | 7.3 | 0.550 | 0.329 | 0.245 |
| PIV3039 | 4.7 | 0.892 | −0.003 | −0.040 |
| PIV3041 | 12.7 | 0.822 | 0.204 | 0.159 |
| PIV3045 | 4.4 | −0.034 | −0.041 | −0.058 |
| PIV3051 | 10.7 | 1.172 | 0.052 | 0.022 |
| PIV3052 | 6.8 | 1.124 | 0.103 | 0.100 |
| PIV3053 | 11.8 | 1.211 | 1.060 | 0.902 |
| PIV3060 | 3.5 | 0.690 | 0.371 | 0.321 |
| PIV3107 | 11.6 | 1.209 | 1.023 | 0.666 |

“ND”—Not Determined.

### 16B: Conformational Integrity of PIV3 F Protein Mutants

Conformation integrity of PIV3 F protein mutants were evaluated by a thermal stress experiment. Crude cell culture supernatants were normalized based on the results of the titer quantitation and then incubated for 1 hour at room temperature and two higher temperatures. For the initial testing of PIV3 F protein mutants, the two temperatures were 50° C. and 53° C. After the incubation, the protein mutants were probed with prefusion specific monoclonal antibody PIA174 (Stewart-Jones et al., Proc Natl Acad Sci USA 115, 12265-12270 (2018)) by OCTET HTX. Anti-Human IgG Fc Capture (AHC) biosensors were first equilibrated in PB before being dipped into more PB to establish the initial baseline. Biosensors were incubated with PIA174 before being equilibrated in PB to establish the experimental baseline. The mAb-bound biosensors were then dipped into the thermal stresses cell culture supernatants for 5 minutes. The kinetics analysis was done by OCTET data analysis software (version 12.0, Sartorius) based on curve fitting of the entire associate step.

The reactivities of the mutants to PIA174 mAb are presented in Table 48. The most stabilizing amino acid substitutions identified from the screens of the individual engineered disulfide mutants, proline mutations, cavity filling mutants, and electrostatic mutations were combined into the combination mutants. The combination mutants were subjected to the thermal stress test at room temperature, 53° C., and 56° C., as shown in Table 49. Notable protein expression and thermal stability improvement were observed for combination mutants.

TABLE 49
Protein expression and thermal stability of PIV3 F protein mutants
comprising engineered disulfide mutations, proline substitution mutations,
glycine replacement mutations, and cavity filling mutations.

| | | Response Shift with PIA174 mAb | | |
|---|---|---|---|---|
| | Protein | No | 53° C. stress | 56° C. stress |
| | expression | stress | resistance | resistance |
| Mutant ID | (mg/L) | (nm) | (nm) | (nm) |
| WT Control | 6.7 | 0.542 | ND | ND |
| PIV3108 | 55.8 | 1.580 | 1.503 | 0.205 |
| PIV3109 | 48.1 | 1.415 | 1.312 | 0.119 |
| PIV3110 | 43.8 | 1.414 | 1.254 | 0.055 |
| PIV3111 | 19.1 | 1.522 | 1.442 | 0.533 |
| PIV3112 | 53.9 | 1.543 | 1.034 | 0.030 |
| PIV3113 | 50.2 | 1.583 | 1.535 | 0.323 |
| PIV3115 | 51.8 | 1.489 | 1.546 | 0.848 |
| PIV3116 | 19.7 | 1.162 | 0.082 | 0.066 |
| PIV3117 | 66.5 | 1.425 | 1.421 | 0.120 |
| PIV3118 | 61.8 | 1.492 | 1.450 | 0.051 |
| PIV3119 | 44.7 | 1.462 | 1.513 | 0.658 |
| PIV3120 | 16.6 | 1.500 | ND | ND |

“ND”—Not Determined.

### Example 17. Expression and Purification of hMPV A F Protein Mutants for Immunogenicity Studies

Expression vectors encoding different hMPV A F protein variants as described in Example 2 were used to transfect Expi293 cells (Thermo Fisher Scientific). Cell culture supernatants were harvested and sterile filtered. The F protein was recovered from the filtrate by Strep-tactin based affinity purification (Strep-Tactin XT, Cytiva). The eluted F protein was further polished by size-exclusion chromatography (SEC, Superdex 200 PG, Cytiva) in 1×PBS. The SEC pool was adjusted to 5% Sucrose and 0.02% PS80, followed by sterile filtration. Similar purification methods were applied across F-protein variants.

### EXAMPLE 18: EXPRESSION AND PURIFICATION OF PIV3 F AND PIV1 F PROTEIN MUTANTS FOR IMMUNOGENICITY STUDIES

Expression vectors encoding different F protein variants as described in Examples 9 and 13 were used to transfect ExpiCHO cells (Thermo Fisher Scientific). The PIV1 F study includes a “literature prefusion comparison” protein control, containing F113G, F114S, A4661, S4731 mutations (Stewart-Jones et al., 2018, PNAS 115, 12265-12270). This PIV1 literature prefusion comparison F protein was produced with the same ectodomain, foldon and C-terminal tags as described in Example 10. Cell culture supernatants were harvested and sterile filtered. The F protein was recovered from the filtrate by Strep-tactin based affinity purification (Strep-Tactin XT, Cytiva). The eluted F protein was further polished by size exclusion chromatography (SEC, Superdex 200 PG, Cytiva) in 1×PBS. The SEC pool was adjusted to 5% Sucrose and 0.02% PS80, followed by sterile filtration. Similar purification methods were applied across F protein variants.

### Example 19: Nucleoside-Modified Messenger RNA (modRNA) Production for Immunogenicity Studies

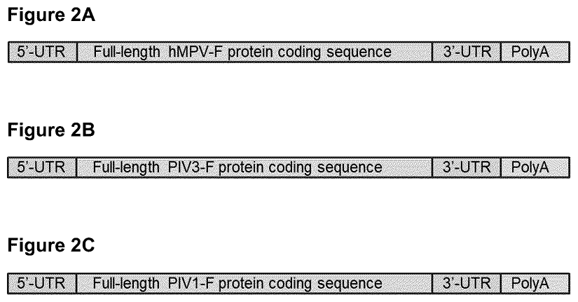

DNA plasmids encoding the full-length F antigens with various engineered mutations were constructed using standard molecular biology techniques. The sequence includes expression elements, such as 5′-untranslated region (5′-UTR), 3′-UTR, and poly-adenosine (poly-A) tail, and a Homo sapiens-codon optimized sequence encoding a full-length F protein with engineered mutations (FIG. 2). The PIV1 F study includes a “literature prefusion comparison” control, containing F113G, F114S, A4661, S4731 mutations within the full-length PIV1 F antigen (Stewart-Jones et al., 2018, PNAS 115, 12265-12270). The same mutant ID is used for modRNA and protein mutant when they comprise the same mutations. However, the structure of the exemplified protein mutant and of the exemplified protein mutant encoded by a modRNA are different as the exemplified modRNAs encode a full length protein mutant comprising the ectodomain, transmembrane domain and cytoplasmic domain of the F protein while the exemplified protein mutants comprise a truncated ectodomain and no intracellular or transmembrane domain. Plasmids were amplified in Escherichia coli and purified using Qiagen Plasmid Maxi kits (Qiagen). Plasmid DNA was linearized immediately following the 3′ end poly-A tail of the modRNA sequence by restriction enzyme digestion and purified by phenol-chloroform. Linearized DNA templates were transcribed into RNA using T7 polymerase, native and 1-methyl pseudouridine (mψU) ribonucleotides, and co-transcriptionally capped using Clean Cap reagent (Trilink). RNA in-vitro transcription reaction was stopped by addition of Turbo DNAse (Thermo Fisher) to digest template DNA and modRNA was purified by LiCI precipitation.

### Example 20: modRNA Formulation into mRNA-Lipid-Nanoparticle (modRNA-LNP) for Immunogenicity Studies

modRNA-LNPs were formulated by combining a modRNA containing aqueous phase and a lipid containing organic phase using a T-mixer. The organic phase was prepared by solubilizing a mixture of ionizable lipid, phospholipid, polyethylene glyco-lipid, and cholesterol at a pre-determined ratio in ethanol. The organic phase and aqueous phase were mixed by syringe pumps. The resultant solution was dialyzed against 10 mM Tris buffer (pH 7.4). Post-dialysis solution was concentrated and spiked with cryo-protectant to a final modRNA-LNP

### Solution

### Example 21: Stabilized hMPV A F Protein Mutants in Prefusion Conformation as Protein Subunit Elicit Neutralizing Responses in Mice

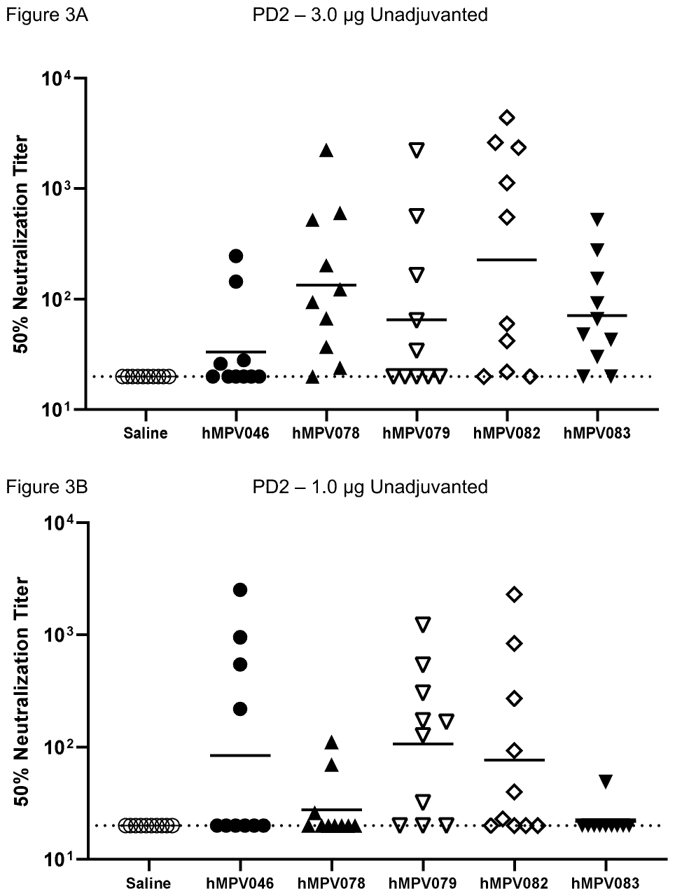

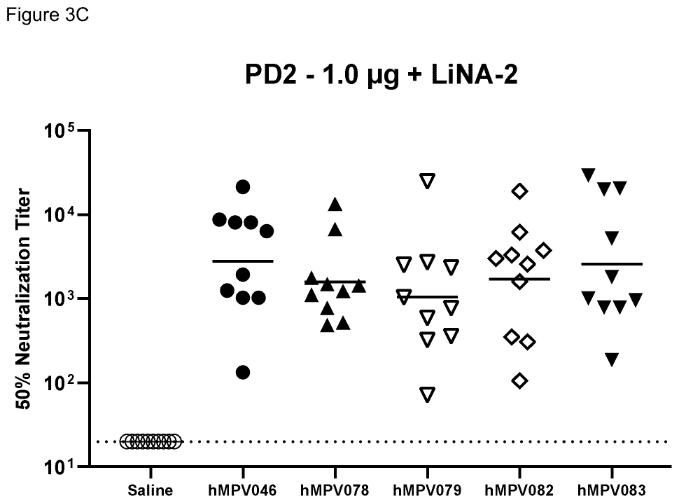

Protein subunit antigens were prepared as described in Example 17 above. Female Balb/c mice were immunized with either 3.0 μg or 1.0 μg of protein subunit with or without LiNA2 (20 μg MPLA,10 μg QS-21 per dose) as adjuvant, of either hMPV A F protein mutants hMPV046, hMPV078, hMPV079, hMPV082, or hMPV083. Immunizations were given intramuscularly at weeks 0 and 3 (Table 52). Post-dose 1 (PD1, week 3) and post-dose 2 (PD2, week 5) sera were evaluated in an hMPV neutralization assay as described with minor modifications (Eyles et al., 2013, J Inf Dis. 208(2):319-29). Briefly, 50% neutralizing titers were determined as the serum dilution factor resulting in a 50% reduction in infectious units. Results are reported as the geometric mean titer (GMT) from 10 mice per group. Sera with no detectable virus neutralization were assigned a titer of 20, the limit of detection of the assay. Fold rise in 50% geometric mean titers are reported as the ratio of post-dose 2 (PD2) of the mutants to the negative control (saline) within each group (Table 53). Overall. all mutants tested elicited a neutralizing response following two immunizations in mice (Table 53, FIG. 3).

TABLE 52
Immunization schedule of the murine immunogenicity
study comparing hMPV A F protein mutants.

| | |
|---|---|
| hMPV A F protein mutant dose | 3.0 μg, 1.0 μg of protein with |
| | and without LiNA-2 |
| Vaccination | Weeks 0, 3 |
| Bleed | Weeks 3 (PD1) |
| | 5 (PD2) |

TABLE 53
Geometric mean and fold-rise of 50% neutralizing titers of Balb/c
mice following two immunizations with hMPV A F protein mutants.

| | 3.0 μg | | 1.0 μg | | 1.0 μg + | |
|---|---|---|---|---|---|---|
| | Unadjuvanted | | Unadjuvanted | | LiNA-2 | |
| Mutant ID | GMT | Fold rise | GMT | Fold rise | GMT | Fold rise |
| hMPV046 | 33 | 1.7 | 84 | 4.2 | 2760 | 138.0 |
| hMPV078 | 135 | 6.8 | 28 | 1.4 | 1570 | 78.5 |
| hMPV079 | 65 | 3.3 | 107 | 5.4 | 1042 | 52.1 |
| hMPV082 | 228 | 11.4 | 77 | 3.9 | 1699 | 85.0 |
| hMPV083 | 71 | 3.6 | 22 | 1.1 | 2566 | 128.3 |
| Saline | 20 | 1.0 | 20 | 1.0 | 20 | 1.0 |

### Example 22: Prefusion-Stabilized, Full-Length hMPV A F Mutants Expressed Through modRNA Elicit Neutralizing Responses in Mice

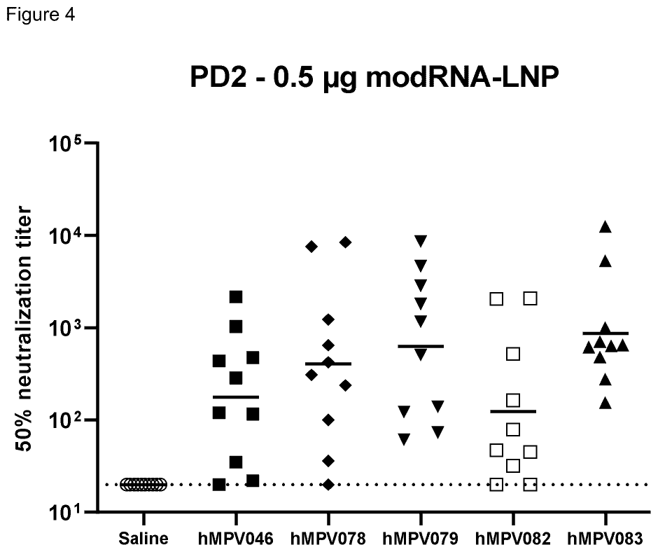

Formulated modRNA-LNPs were prepared as described in Example 19 and 20 above. One lower dose level was selected in order to provide more sensitive results and potentially differentiate different designs. Female Balb/c mice were immunized with 0.5 μg of LNP-formulated modRNA encoding full-length hMPV A F protein mutants hMPV046, hMPV078, hMPV079, hMPV082, or hMPV083. Immunizations were given intramuscularly at weeks 0 and 3 (Table 54). Post-dose 1 (PD1, week 3) and post-dose 2 (PD2, week 5) sera were evaluated in an hMPV neutralization assay as described with minor modifications (Eyles et al., 2013, J Inf Dis. 208(2):319-29). Briefly, neutralizing titers were determined as the serum dilution factor resulting in a 50% reduction in infectious units. Results are reported as the geometric mean titer from 10 mice per group. Fold rise in 50% geometric mean titers are reported as the ratio of post-dose 2 (PD2) of the mutants to the negative control (saline) titer within each group (Table 55). Overall, all mutants tested elicited a neutralizing response following two immunizations in mice compared with the saline reference (FIG. 4). hMPV078, hMPV079, and hMPV083 showed notably higher neutralizing GMTs, suggesting these mutants are the more immunogenic forms of a stabilized hMPV A prefusion antigen encoded from modRNA.

TABLE 54
Immunization schedule of the murine immunogenicity study comparing
full-length hMPV A F protein mutants encoded from modRNA.

| | |
|---|---|
| hMPV A F protein mutant | 0.5 μg modRNA-LNP |
| modRNA dose | |
| Vaccination | Weeks 0, 3 |
| Bleed | Weeks 3 (PD1) |
| | 5 (PD2) |

TABLE 55
Geometric mean and fold-rise of 50% neutralizing
titers of Balb/c mice following two immunizations
with modRNA-LNP of hMPV A F mutants.

0.5 μg modRNA-LNP

| Mutant ID | GMT | Fold rise |
|---|---|---|
| hMPV046 | 176 | 8.8 |
| hMPV078 | 403 | 20.2 |
| hMPV079 | 626 | 31.3 |
| hMPV082 | 123 | 6.2 |
| hMPV083 | 869 | 43.5 |
| Saline | 20 | 1.0 |

### Example 23: Stabilized PIV3 F Protein Mutants in Prefusion Conformation as Protein Subunit Elicit Neutralizing Responses in Mice

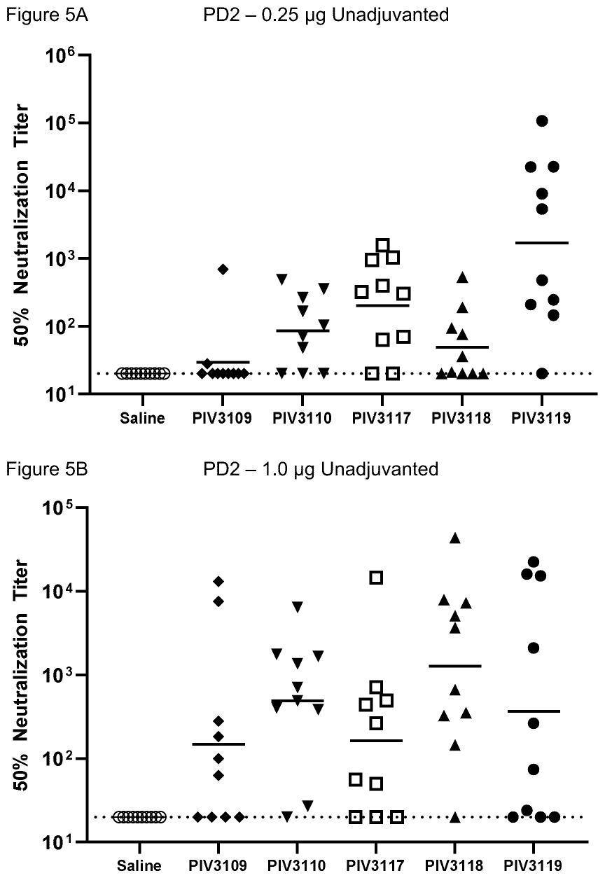

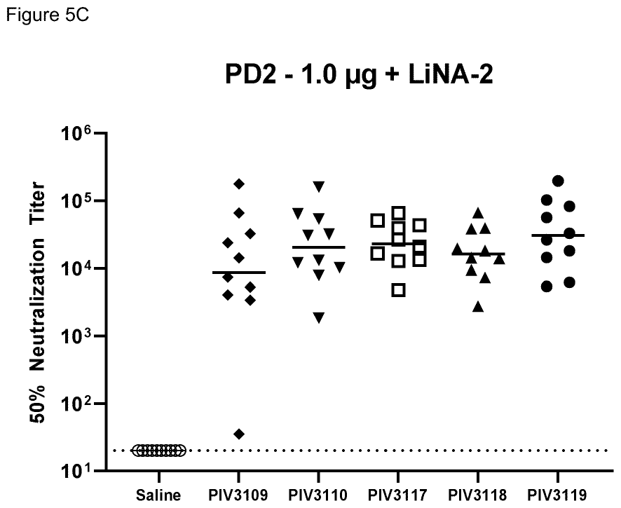

Protein subunit antigens were prepared as described in Example 18 above. Female Balb/c mice were immunized with either 0.25 μg or 1.0 μg of protein subunit with or without LiNA2 (20 μg MPLA, 10 μg QS-21 per dose) as adjuvant, of either PIV3 F protein mutants PIV3109, PIV3110, PIV3117, PIV3118, or PIV3119. Immunizations were given intramuscularly at weeks 0 and 3 (Table 56). Post-dose 1 (PD1, week 3) and post-dose 2 (PD2, week 5) sera were evaluated in an PIV3 neutralization assay as described with minor modifications (Eyles et al., 2013, J Inf Dis. 208(2):319-29). Briefly, neutralizing titers were determined as the serum dilution factor resulting in a 50% reduction in infectious units. Results are reported as the geometric mean titer from 10 mice per group. Sera with no detectable virus neutralization were assigned a titer of 20. Fold rise in 50% geometric mean titers are reported as the ratio of post-dose 2 (PD2) of the mutants to the negative control (saline) within each group. Overall, all mutants tested elicited a neutralizing response following two immunizations in mice (Table 57, FIG. 5). At 1.0 μg with adjuvant, PIV3110, PIV3117 and PIV3119 showed the highest neutralizing titers. At 1.0 μg dose without adjuvant, PIV3110, PIV3118 and PIV3119 are higher than the other two mutants, whereas at the lower 0.25 μg dose, PIV3117 and PIV3119 elicit higher neutralizing titers than the other antigens tested in the study.

TABLE 56
Immunization schedule of the murine immunogenicity
study comparing PIV3 F protein mutants.

| | |
|---|---|
| PIV3 F protein mutant dose | 0.25 μg, 1.0 μg of protein with |
| | and without LiNA-2 |
| Vaccination | Weeks 0, 3 |
| Bleed | Weeks 3 (PD1) |
| | 5 (PD2) |

TABLE 57
Geometric mean and fold-rise of 50% neutralizing titers of Balb/c
mice following two immunizations with PIV3 F protein mutants.

| | 0.25 μg | | 1.0 μg | | 1.0 μg + | |
|---|---|---|---|---|---|---|
| | Unadjuvanted | | Unadjuvanted | | LiNA-2 | |
| Mutant ID | GMT | Fold rise | GMT | Fold rise | GMT | Fold rise |
| PIV3109 | 29 | 1.5 | 148 | 7.4 | 15998 | 799.9 |
| PIV3110 | 86 | 4.3 | 487 | 24.4 | 20460 | 1023.0 |
| PIV3117 | 200 | 10.0 | 163 | 8.2 | 23128 | 1156.4 |
| PIV3118 | 49 | 2.5 | 1269 | 63.5 | 16358 | 817.9 |
| PIV3119 | 1683 | 84.2 | 366 | 18.3 | 30626 | 1531.3 |
| Saline | 20 | 1.0 | 20 | 1.0 | 20 | 1.0 |

### Example 24: Prefusion-Stabilized, Full-Length PIV3 F Protein Mutants Expressed Through modRNA Elicit Neutralizing Responses in Mice

Formulated modRNA-LNPs were prepared as described in Example 19 and 20 above. Two lower dose levels were selected in order to provide more sensitive results and potentially differentiate different designs. Female Balb/c mice were immunized with 0.05 μg or 0.2 μg of LNP-formulated modRNA encoding full-length PIV3 F protein mutants PIV3109, PIV3110, PIV3117, PIV3118, or PIV3119. Immunizations were given intramuscularly at weeks 0 and 3 (Table 58). Post-dose 1 (PD1, week 3) and post-dose 2 (PD2, week 5) sera were evaluated in an PIV3 neutralization assay as described with minor modifications (Eyles et al., 2013, J Inf Dis. 208(2):319-29). Briefly, neutralizing titers were determined as the serum dilution factor resulting in a 50% reduction in infectious units. Results are reported as the geometric mean titer from 10 mice per group. Fold rise in 50% geometric mean titers are reported as the ratio of post-dose 2 (PD2) of the mutants to the negative control (saline) titer within each group (Table 59).

TABLE 58
Immunization schedule of the murine immunogenicity study comparing
full-length PIV3 F protein mutants encoded from modRNA.

| | |
|---|---|
| PIV3 F protein mutant | 0.05 μg, 0.2 μg modRNA-LNP |
| modRNA dose | |
| Vaccination | Weeks 0, 3 |
| Bleed | Weeks 3 (PD1) |
| | 5 (PD2) |

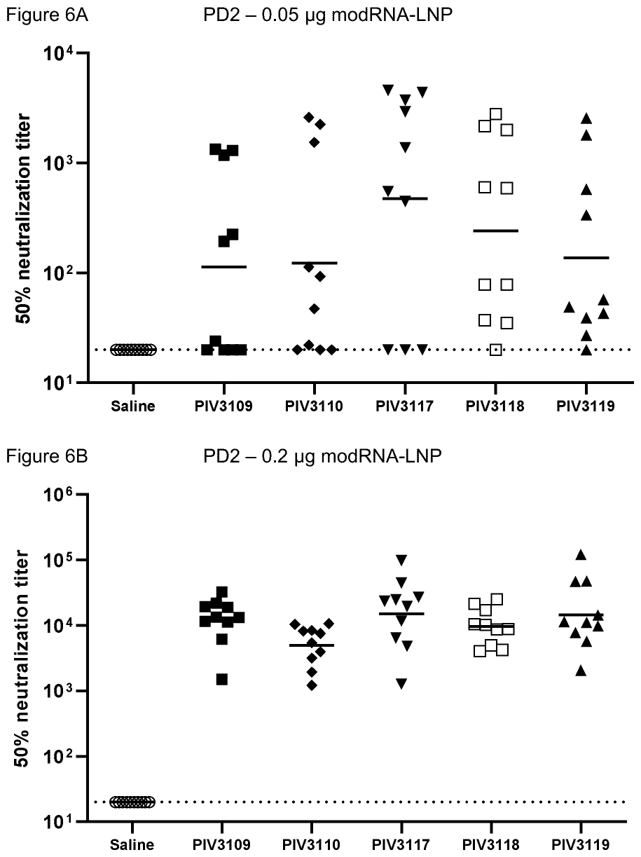

All mutants tested elicited a neutralizing response following two immunizations in mice compared with the saline reference (FIG. 6). For 0.2 μg dose, antibody titers of PD2 were higher for mutants PIV3109, PIV3117, and PIV3119, suggesting these mutants were the more immunogenic form of a stabilized PIV3 F prefusion antigens expressed through modRNA (Table 59, FIG. 6).

TABLE 59
Geometric mean and fold-rise of 50% neutralizing
titers of Balb/c mice following two immunizations
with modRNA-LNP of PIV3 F mutants.

| | 0.05 μg modRNA-LNP | | 0.2 μg modRNA-LNP | |
|---|---|---|---|---|
| Mutant ID | GMT | Fold rise | GMT | Fold rise |
| PIV3109 | 113 | 5.7 | 11843 | 592.2 |
| PIV3110 | 123 | 6.2 | 4962 | 248.1 |
| PIV3117 | 475 | 23.8 | 15052 | 752.6 |
| PIV3118 | 241 | 12.1 | 9546 | 477.3 |
| PIV3119 | 137 | 6.9 | 14606 | 730.3 |
| Saline | 20 | 1.0 | 20 | 1.0 |

### Example 25: Stabilized PIV1 F Protein Mutants in Prefusion Conformation as Protein Subunit Elicit Neutralizing Responses in Mice

Protein subunit antigens were prepared as described in Example 18 above. Female Balb/c mice were immunized with either 2.0 μg or 0.5 μg of protein subunit with or without LiNA-2 (20 μg MPLA, 10 μg QS-21 per dose) as adjuvant, of either literature prefusion comparison PIV1 F protein, F protein mutants PIV1047, PIV1053, PIV1054, or PIV1069. The literature prefusion comparison design is defined earlier in Example 18 (Stewart-Jones et al., 2018, PNAS 115, 12265-12270). Immunizations were given intramuscularly at weeks 0 and 3 (Table 60). Post-dose 1 (PD1, week 3) and post-dose 2 (PD2, week 5) sera were evaluated in an PIV1 neutralization assay as described with minor modifications (Eyles et al., 2013, J Inf Dis. 208(2):319-29). Briefly, neutralizing titers were determined as the serum dilution factor resulting in a 50% reduction in infectious units. Results are reported as the geometric mean titer from 10 mice per group. Fold rise in 50% geometric mean titers are reported as the ratio of post-dose 2 (PD2) of the mutants to the negative control (saline) titer within each group.

TABLE 60
Immunization schedule of the murine immunogenicity
study comparing PIV1 F protein mutants.

| | |
|---|---|
| PIV1 F protein mutants dose | 2.0 μg, 0.5 μg of protein with |
| | and without LiNA-2 |
| Vaccination | Weeks 0, 3 |
| Bleed | Weeks 3 (PD1) |
| | 5 (PD2) |

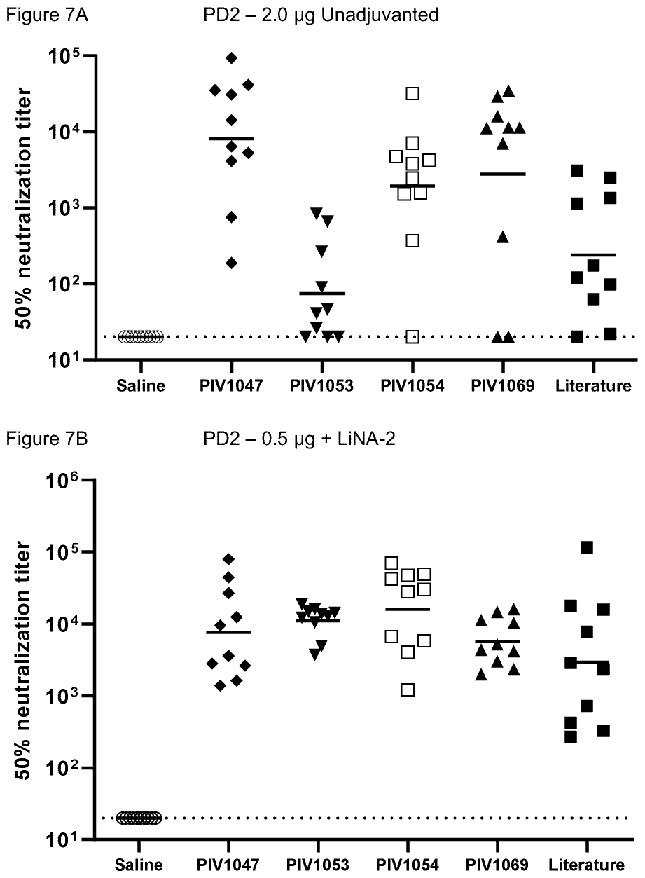

All mutants tested elicited neutralizing responses following two immunizations in mice compared with the saline control group (FIG. 7). Overall, antibody titers of PD2 were consistently higher with both 2.0 μg unadjuvanted and 0.5 μg adjuvanted (with LiNA-2) formulations for mutants PIV1047, PIV1054, and PIV1069 than the literature prefusion comparison design, suggesting these mutants were the more immunogenic form of a stabilized PIV1 F prefusion protein (Table 61, FIG. 7).

TABLE 61
Geometric mean and fold-rise of 50% neutralizing titers of Balb/c
mice following two immunizations with PIV1 F protein mutants.

| | 2.0 μg Unadjuvanted | | 0.5 μg + LiNA-2 | |
|---|---|---|---|---|
| Mutant ID | GMT | Fold rise | GMT | Fold rise |
| PIV1047 | 8112 | 405.6 | 7619 | 381.0 |
| PIV1053 | 74 | 3.7 | 10996 | 549.8 |
| PIV1054 | 1941 | 97.1 | 16002 | 800.1 |
| PIV1069 | 2784 | 139.2 | 5715 | 285.8 |
| Literature prefusion | 241 | 12.1 | 2925 | 146.3 |
| comparison | | | | |
| Saline | 20 | 1.0 | 20 | 1.0 |

### Example 26: Prefusion-Stabilized, Full-Length PIV1 F Protein Mutants Expressed Through modRNA Elicit Neutralizing Responses in Mice

Formulated modRNA-LNPs were prepared as described in Example 19 and 20 above. One lower dose level was selected in order to provide more sensitive results and potentially differentiate different designs. Female Balb/c mice were immunized with 0.2 μg of LNP-formulated modRNA encoding either a literature prefusion comparison F design or full length PIV1 F protein mutants PIV1047, PIV1053, PIV1054, or PIV1069. The literature prefusion comparison design is defined earlier in Example 18 (Stewart-Jones et al., 2018, PNAS 115, 12265-12270). Immunizations were given intramuscularly at weeks 0 and 3 (Table 62). Post-dose 1 (PD1, week 3) and post-dose 2 (PD2, week 5) sera were evaluated in an PIV1 neutralization assay as described with minor modifications (Eyles et al., 2013, J Inf Dis. 208(2):319-29). Briefly, neutralizing titers were determined as the serum dilution factor resulting in a 50% reduction in infectious units. Results are reported as the geometric mean titer from 10 mice per group. Fold rise in 50% geometric mean titers are reported as the ratio of post-dose 2 (PD2) of the mutants to the negative control (saline) titer within each group (Table 63).

TABLE 62
Immunization schedule of the murine immunogenicity study comparing
full-length PIV1 F protein mutants encoded from modRNA.

| | |
|---|---|
| PIV1 F protein mutant | 0.2 μg modRNA-LNP |
| modRNA dose | |
| Vaccination | Weeks 0, 3 |
| Bleed | Weeks 3 (PD1) |
| | 5 (PD2) |

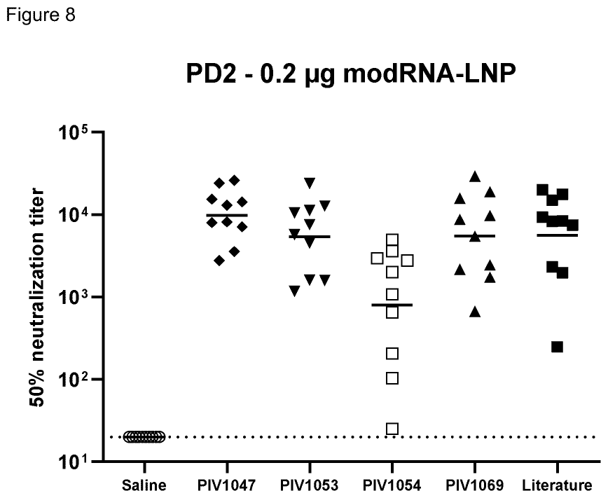

All mutants tested elicited a neutralizing response following two immunizations in mice compared with the saline reference (FIG. 8). Overall, antibody titers of PD2 were comparable or trending higher for mutants PIV1047, PIV1053, and PIV1069 than the literature prefusion comparison design, suggesting these mutants were the more immunogenic form of a stabilized PIV1 F prefusion antigens expressed through modRNA (Table 63, FIG. 8).

TABLE 63
Geometric mean and fold-rise of 50% neutralizing
titers of Balb/c mice following two immunizations
with modRNA-LNP of PIV1 F mutants.

0.2 μg modRNA-LNP

| Mutant ID | GMT | Fold rise |
|---|---|---|
| PIV1047 | 9779 | 489.0 |
| PIV1053 | 5371 | 268.6 |
| PIV1054 | 800 | 40.0 |
| PIV1069 | 5485 | 274.3 |
| Literature prefusion | 5563 | 278.2 |
| comparison | | |
| Saline | 20 | 1.0 |

### Example 27: Design and Preparation of Additional PIV3 F Protein Mutants

This example illustrates the design and preparation of various PIV3 F protein mutants, which include a fibritin foldon and introduced amino acid mutations, such as engineered disulfide bond mutations, cavity filling mutations, electrostatic mutations, cleavage site mutations, or a combination thereof. Exemplary PIV3 F protein mutants, each of which is identified by an unique identifier, such as PIV3134, PIV3135, etc., are provided in Tables 64-69. Each of these mutants was designed and prepared based on the amino acid sequence set forth in SEQ ID NO:305 or 420. Amino acid residues 1-481 of the sequence of SEQ ID NO:305 are identical to amino acid residues 1-481 of the F0 precursor polypeptide of native PIV3 as set forth in SEQ ID NO:300. For PIV3 F protein mutants comprising extended ectodomain, amino acid residues 1-484 of the sequence of SEQ ID NO:420 are identical to amino acid residues 1-484 of the F0 precursor polypeptide of native PIV3 as set forth in SEQ ID NO:300. Therefore, the amino acid sequences of these exemplary F mutants are identical except for the introduced amino acid mutations as noted for each mutant listed in Tables 64-69. Each of these PIV3 F protein mutants comprises two polypeptide chains. One of the polypeptide chains, the F2 polypeptide, comprises amino acids 19-109 of SEQ ID NO:305 except for the introduced mutations as noted. The other polypeptide chain comprises the F1 polypeptide (residues 110-481) of SEQ ID NO:305 except for the introduced mutations as noted linked to a foldon trimerization domain (residues 486-512) via a GGGS linker (residues 482-485). The F1 polypeptide (residues 110-484) of the mutants comprising extended ectodomain are linked to a foldon trimerization domain (residues 489-515) via a GGGS linker (residues 485-488). The signal peptide (residues 1-18) of SEQ ID NO:305 and 420 were cleaved from the F0 precursor during the expression process.

TABLE 64
Exemplary PIV3 F Protein Mutants Comprising Engineered Disulfide Mutations

| | | Amino Acid Sequence (residues 110-481 for F1 polypeptide and |
|---|---|---|
| Mutant ID | Mutations | residues 19-109 for F2 polypeptide) |
| PIV3135 | E209C- | F1 (SEQ ID NO: 423): |
| | L234C | FFGGVIGTIALGVATSAQITAAVALVEAKQARSDIEKLKEAIRDTN |
| | | KAVQSVQSSVGNLIVAIKSVQDYVNKEIVPSIARLGCEAAGLQLG |
| | | IALTQHYSCLTNIFGDNIGSLQEKGIKLQGIASCYRTNITEIFTTST |
| | | VDKYDIYDLLFTESIKVRVIDVDLNDYSITLQVRLPLLTRLLNTQIY |
| | | KVDSISYNIQNREWYIPLPSHIMTKGAFLGGADVKECIEAFSSYIC |
| | | PSDPGFVLNHEMESCLSGNISQCPRTTVTSDIVPRYAFVNGGV |
| | | VANCITTTCTCNGIGNRINQPPDQGVKIITHKECNTIGINGMLFNT |
| | | NKEGTLAFYTPDDITLNNSVALDPIDISIELNKAKSDLEESKEWIR |
| | | RSNQKL |
| | | F2 (SEQ ID NO: 424): |
| | | QIDITKLQHVGVLVNSPKGMKISQNFETRYLILSLIPKIEDSNSCG |
| | | DQQIKQYKRLLDRLIIPLYDGLRLQKDVIVTNQESNENTDPRTER |
| PIV3136 | E209C- | F1 (SEQ ID NO: 425): |
| | S233C | FFGGVIGTIALGVATSAQITAAVALVEAKQARSDIEKLKEAIRDTN |
| | | KAVQSVQSSVGNLIVAIKSVQDYVNKEIVPSIARLGCEAAGLQLG |
| | | IALTQHYSCLTNIFGDNIGSLQEKGIKLQGIACLYRTNITEIFTTST |
| | | VDKYDIYDLLFTESIKVRVIDVDLNDYSITLQVRLPLLTRLLNTQIY |
| | | KVDSISYNIQNREWYIPLPSHIMTKGAFLGGADVKECIEAFSSYIC |
| | | PSDPGFVLNHEMESCLSGNISQCPRTTVTSDIVPRYAFVNGGV |
| | | VANCITTTCTCNGIGNRINQPPDQGVKIITHKECNTIGINGMLFNT |
| | | NKEGTLAFYTPDDITLNNSVALDPIDISIELNKAKSDLEESKEWIR |
| | | RSNQKL |
| | | F2 (SEQ ID NO: 426): |
| | | QIDITKLQHVGVLVNSPKGMKISQNFETRYLILSLIPKIEDSNSCG |
| | | DQQIKQYKRLLDRLIIPLYDGLRLQKDVIVTNQESNENTDPRTER |
| PIV3137 | G85C- | F1 (SEQ ID NO: 427): |
| | E209C | FFGGVIGTIALGVATSAQITAAVALVEAKQARSDIEKLKEAIRDTN |
| | | KAVQSVQSSVGNLIVAIKSVQDYVNKEIVPSIARLGCEAAGLQLG |
| | | IALTQHYSCLTNIFGDNIGSLQEKGIKLQGIASLYRTNITEIFTTST |
| | | VDKYDIYDLLFTESIKVRVIDVDLNDYSITLQVRLPLLTRLLNTQIY |
| | | KVDSISYNIQNREWYIPLPSHIMTKGAFLGGADVKECIEAFSSYIC |
| | | PSDPGFVLNHEMESCLSGNISQCPRTTVTSDIVPRYAFVNGGV |
| | | VANCITTTCTCNGIGNRINQPPDQGVKIITHKECNTIGINGMLFNT |
| | | NKEGTLAFYTPDDITLNNSVALDPIDISIELNKAKSDLEESKEWIR |
| | | RSNQKL |
| | | F2 (SEQ ID NO: 428): |
| | | QIDITKLQHVGVLVNSPKGMKISQNFETRYLILSLIPKIEDSNSCG |
| | | DQQIKQYKRLLDRLIIPLYDCLRLQKDVIVTNQESNENTDPRTER |

TABLE 65
Exemplary PIV3 F Protein Mutants Comprising Cavity Filing Mutations or
Electrostatic Mutations

| Mutant | | Amino Acid Sequence (residues 110-481 for F1 polypeptide and |
|---|---|---|
| ID | Mutations | residues 19-109 for F2 polypeptide) |
| PIV3134 | T277V | F1 (SEQ ID NO: 429): |
| | | FFGGVIGTIALGVATSAQITAAVALVEAKQARSDIEKLKEAIRDTNK |
| | | AVQSVQSSVGNLIVAIKSVQDYVNKEIVPSIARLGCEAAGLQLGIA |
| | | LTQHYSELTNIFGDNIGSLQEKGIKLQGIASLYRTNITEIFTTSTVDK |
| | | YDIYDLLFTESIKVRVIDVDLNDYSIVLQVRLPLLTRLLNTQIYKVDS |
| | | ISYNIQNREWYIPLPSHIMTKGAFLGGADVKECIEAFSSYICPSDP |
| | | GFVLNHEMESCLSGNISQCPRTTVTSDIVPRYAFVNGGVVANCIT |
| | | TTCTCNGIGNRINQPPDQGVKIITHKECNTIGINGMLFNTNKEGTL |
| | | AFYTPDDITLNNSVALDPIDISIELNKAKSDLEESKEWIRRSNQKL |
| | | F2 (SEQ ID NO: 430): |
| | | QIDITKLQHVGVLVNSPKGMKISQNFETRYLILSLIPKIEDSNSCGD |
| | | QQIKQYKRLLDRLIIPLYDGLRLQKDVIVTNQESNENTDPRTER |
| PIV3138 | A463L, | F1 (SEQ ID NO: 431): |
| | S470L | FFGGVIGTIALGVATSAQITAAVALVEAKQARSDIEKLKEAIRDTNK |
| | | AVQSVQSSVGNLIVAIKSVQDYVNKEIVPSIARLGCEAAGLQLGIA |
| | | LTQHYSELTNIFGDNIGSLQEKGIKLQGIASLYRTNITEIFTTSTVDK |
| | | YDIYDLLFTESIKVRVIDVDLNDYSITLQVRLPLLTRLLNTQIYKVDSI |
| | | SYNIQNREWYIPLPSHIMTKGAFLGGADVKECIEAFSSYICPSDPG |
| | | FVLNHEMESCLSGNISQCPRTTVTSDIVPRYAFVNGGVVANCITT |
| | | TCTCNGIGNRINQPPDQGVKIITHKECNTIGINGMLFNTNKEGTLA |
| | | FYTPDDITLNNSVALDPIDISIELNKLKSDLEELKEWIRRSNQKL |
| | | F2 (SEQ ID NO: 432): |
| | | QIDITKLQHVGVLVNSPKGMKISQNFETRYLILSLIPKIEDSNSCGD |
| | | QQIKQYKRLLDRLIIPLYDGLRLQKDVIVTNQESNENTDPRTER |
| PIV3139 | A463L, | F1 (SEQ ID NO: 433): |
| | I474F | FFGGVIGTIALGVATSAQITAAVALVEAKQARSDIEKLKEAIRDTNK |
| | | AVQSVQSSVGNLIVAIKSVQDYVNKEIVPSIARLGCEAAGLQLGIA |
| | | LTQHYSELTNIFGDNIGSLQEKGIKLQGIASLYRTNITEIFTTSTVDK |
| | | YDIYDLLFTESIKVRVIDVDLNDYSITLQVRLPLLTRLLNTQIYKVDSI |
| | | SYNIQNREWYIPLPSHIMTKGAFLGGADVKECIEAFSSYICPSDPG |
| | | FVLNHEMESCLSGNISQCPRTTVTSDIVPRYAFVNGGVVANCITT |
| | | TCTCNGIGNRINQPPDQGVKIITHKECNTIGINGMLFNTNKEGTLA |
| | | FYTPDDITLNNSVALDPIDISIELNKLKSDLEESKEWFRRSNQKL |
| | | F2 (SEQ ID NO: 434): |
| | | QIDITKLQHVGVLVNSPKGMKISQNFETRYLILSLIPKIEDSNSCGD |
| | | QQIKQYKRLLDRLIIPLYDGLRLQKDVIVTNQESNENTDPRTER |
| PIV3152 | A4631, | F1 (SEQ ID NO: 435): |
| | S4701 | FFGGVIGTIALGVATSAQITAAVALVEAKQARSDIEKLKEAIRDTNK |
| | | AVQSVQSSVGNLIVAIKSVQDYVNKEIVPSIARLGCEAAGLQLGIA |
| | | LTQHYSELTNIFGDNIGSLQEKGIKLQGIASLYRTNITEIFTTSTVDK |
| | | YDIYDLLFTESIKVRVIDVDLNDYSITLQVRLPLLTRLLNTQIYKVDSI |
| | | SYNIQNREWYIPLPSHIMTKGAFLGGADVKECIEAFSSYICPSDPG |
| | | FVLNHEMESCLSGNISQCPRTTVTSDIVPRYAFVNGGVVANCITT |
| | | TCTCNGIGNRINQPPDQGVKIITHKECNTIGINGMLFNTNKEGTLA |
| | | FYTPDDITLNNSVALDPIDISIELNKIKSDLEEIKEWIRRSNQKL |
| | | F2 (SEQ ID NO: 436): |
| | | QIDITKLQHVGVLVNSPKGMKISQNFETRYLILSLIPKIEDSNSCGD |
| | | QQIKQYKRLLDRLIIPLYDGLRLQKDVIVTNQESNENTDPRTER |

TABLE 66
Exemplary PIV3 F Protein Mutants Comprising Engineered Disulfide Mutations,
Electrostatic Mutations, and Cavity Filling Mutations

| Mutant | | Amino Acid Sequence (residues 110-481 for F1 polypeptide |
|---|---|---|
| ID | Mutations | and residues 19-109 for F2 polypeptide) |
| PIV3140 | S160C-V170C, | F1 (SEQ ID NO: 437): |
| | E209C-L234C, | FFGGVIGTIALGVATSAQITAAVALVEAKQARSDIEKLKEAIRD |
| | A463L, S470L | TNKAVQCVQSSVGNLICAIKSVQDYVNKEIVPSIARLGCEAAG |
| | | LQLGIALTQHYSCLTNIFGDNIGSLQEKGIKLQGIASCYRTNIT |
| | | EIFTTSTVDKYDIYDLLFTESIKVRVIDVDLNDYSITLQVRLPLL |
| | | TRLLNTQIYKVDSISYNIQNREWYIPLPSHIMTKGAFLGGADV |
| | | KECIEAFSSYICPSDPGFVLNHEMESCLSGNISQCPRTTVTS |
| | | DIVPRYAFVNGGVVANCITTTCTCNGIGNRINQPPDQGVKIIT |
| | | HKECNTIGINGMLFNTNKEGTLAFYTPDDITLNNSVALDPIDISI |
| | | ELNKLKSDLEELKEWIRRSNQKL |
| | | F2 (SEQ ID NO: 438): |
| | | QIDITKLQHVGVLVNSPKGMKISQNFETRYLILSLIPKIEDSNS |
| | | CGDQQIKQYKRLLDRLIIPLYDGLRLQKDVIVTNQESNENTDP |
| | | RTER |
| PIV3141 | S160C-V170C, | F1 (SEQ ID NO: 439): |
| | E209C-L234C, | FFGGVIGTIALGVATSAQITAAVALVEAKQARSDIEKLKEAIRD |
| | A463L, I474F | TNKAVQCVQSSVGNLICAIKSVQDYVNKEIVPSIARLGCEAAG |
| | | LQLGIALTQHYSCLTNIFGDNIGSLQEKGIKLQGIASCYRTNIT |
| | | EIFTTSTVDKYDIYDLLFTESIKVRVIDVDLNDYSITLQVRLPLL |
| | | TRLLNTQIYKVDSISYNIQNREWYIPLPSHIMTKGAFLGGADV |
| | | KECIEAFSSYICPSDPGFVLNHEMESCLSGNISQCPRTTVTS |
| | | DIVPRYAFVNGGVVANCITTTCTCNGIGNRINQPPDQGVKIIT |
| | | HKECNTIGINGMLFNTNKEGTLAFYTPDDITLNNSVALDPIDISI |
| | | ELNKLKSDLEESKEWFRRSNQKL |
| | | F2 (SEQ ID NO: 440): |
| | | QIDITKLQHVGVLVNSPKGMKISQNFETRYLILSLIPKIEDSNS |
| | | CGDQQIKQYKRLLDRLIIPLYDGLRLQKDVIVTNQESNENTDP |
| | | RTER |
| PIV3142 | S160C-V170C, | F1 (SEQ ID NO: 441): |
| | E209C-S233C, | FFGGVIGTIALGVATSAQITAAVALVEAKQARSDIEKLKEAIRD |
| | A463L, S470L | TNKAVQCVQSSVGNLICAIKSVQDYVNKEIVPSIARLGCEAAG |
| | | LOLGIALTQHYSCLTNIFGDNIGSLQEKGIKLQGIACLYRTNIT |
| | | EIFTTSTVDKYDIYDLLFTESIKVRVIDVDLNDYSITLQVRLPLL |
| | | TRLLNTQIYKVDSISYNIQNREWYIPLPSHIMTKGAFLGGADV |
| | | KECIEAFSSYICPSDPGFVLNHEMESCLSGNISQCPRTTVTS |
| | | DIVPRYAFVNGGVVANCITTTCTCNGIGNRINQPPDQGVKIIT |
| | | HKECNTIGINGMLFNTNKEGTLAFYTPDDITLNNSVALDPIDISI |
| | | ELNKLKSDLEELKEWIRRSNQKL |
| | | F2 (SEQ ID NO: 442): |
| | | QIDITKLQHVGVLVNSPKGMKISQNFETRYLILSLIPKIEDSNS |
| | | CGDQQIKQYKRLLDRLIIPLYDGLRLQKDVIVTNQESNENTDP |
| | | RTER |
| PIV3143 | S160C-V170C, | F1 (SEQ ID NO: 443): |
| | E209C-S233C, | FFGGVIGTIALGVATSAQITAAVALVEAKQARSDIEKLKEAIRD |
| | A463L, I474F | TNKAVQCVQSSVGNLICAIKSVQDYVNKEIVPSIARLGCEAAG |
| | | LQLGIALTQHYSCLTNIFGDNIGSLQEKGIKLQGIACLYRTNIT |
| | | EIFTTSTVDKYDIYDLLFTESIKVRVIDVDLNDYSITLQVRLPLL |
| | | TRLLNTQIYKVDSISYNIQNREWYIPLPSHIMTKGAFLGGADV |
| | | KECIEAFSSYICPSDPGFVLNHEMESCLSGNISQCPRTTVTS |
| | | DIVPRYAFVNGGVVANCITTTCTCNGIGNRINQPPDQGVKIIT |
| | | HKECNTIGINGMLFNTNKEGTLAFYTPDDITLNNSVALDPIDISI |
| | | ELNKLKSDLEESKEWFRRSNQKL |
| | | F2 (SEQ ID NO: 444): |
| | | QIDITKLQHVGVLVNSPKGMKISQNFETRYLILSLIPKIEDSNS |
| | | CGDQQIKQYKRLLDRLIIPLYDGLRLQKDVIVTNQESNENTDP |
| | | RTER |
| PIV3144 | S160C-V170C, | F1 (SEQ ID NO: 445): |
| | G85C-E209C, | FFGGVIGTIALGVATSAQITAAVALVEAKQARSDIEKLKEAIRD |
| | A463L, S470L | TNKAVQCVQSSVGNLICAIKSVQDYVNKEIVPSIARLGCEAAG |
| | | LQLGIALTQHYSCLTNIFGDNIGSLQEKGIKLQGIASLYRTNITE |
| | | IFTTSTVDKYDIYDLLFTESIKVRVIDVDLNDYSITLQVRLPLLT |
| | | RLLNTQIYKVDSISYNIQNREWYIPLPSHIMTKGAFLGGADVK |
| | | ECIEAFSSYICPSDPGFVLNHEMESCLSGNISQCPRTTVTSDI |
| | | VPRYAFVNGGVVANCITTTCTCNGIGNRINQPPDQGVKIITHK |
| | | ECNTIGINGMLFNTNKEGTLAFYTPDDITLNNSVALDPIDISIEL |
| | | NKLKSDLEELKEWIRRSNQKL |
| | | F2 (SEQ ID NO: 446): |
| | | QIDITKLQHVGVLVNSPKGMKISQNFETRYLILSLIPKIEDSNS |
| | | CGDQQIKQYKRLLDRLIIPLYDCLRLQKDVIVTNQESNENTDP |
| | | RTER |
| PIV3145 | S160C-V170C, | F1 (SEQ ID NO: 447): |
| | G85C-E209C, | FFGGVIGTIALGVATSAQITAAVALVEAKQARSDIEKLKEAIRD |
| | A463L, I474F | TNKAVQCVQSSVGNLICAIKSVQDYVNKEIVPSIARLGCEAAG |
| | | LQLGIALTQHYSCLTNIFGDNIGSLQEKGIKLQGIASLYRTNITE |
| | | IFTTSTVDKYDIYDLLFTESIKVRVIDVDLNDYSITLQVRLPLLT |
| | | RLLNTQIYKVDSISYNIQNREWYIPLPSHIMTKGAFLGGADVK |
| | | ECIEAFSSYICPSDPGFVLNHEMESCLSGNISQCPRTTVTSDI |
| | | VPRYAFVNGGVVANCITTTCTCNGIGNRINQPPDQGVKIITHK |
| | | ECNTIGINGMLFNTNKEGTLAFYTPDDITLNNSVALDPIDISIEL |
| | | NKLKSDLEESKEWFRRSNQKL |
| | | F2 (SEQ ID NO: 448): |
| | | QIDITKLQHVGVLVNSPKGMKISQNFETRYLILSLIPKIEDSNS |
| | | CGDQQIKQYKRLLDRLIIPLYDCLRLQKDVIVTNQESNENTDP |
| | | RTER |
| PIV3146 | S160C-V170C, | F1 (SEQ ID NO: 449): |
| | E209C-L234C, | FFGGVIGTIALGVATSAQITAAVALVEAKQARSDIEKLKEAIRD |
| | T277V, A463L, | TNKAVQCVQSSVGNLICAIKSVQDYVNKEIVPSIARLGCEAAG |
| | S470L | LQLGIALTQHYSCLTNIFGDNIGSLQEKGIKLQGIASCYRTNIT |
| | | EIFTTSTVDKYDIYDLLFTESIKVRVIDVDLNDYSIVLQVRLPLL |
| | | TRLLNTQIYKVDSISYNIQNREWYIPLPSHIMTKGAFLGGADV |
| | | KECIEAFSSYICPSDPGFVLNHEMESCLSGNISQCPRTTVTS |
| | | DIVPRYAFVNGGVVANCITTTCTCNGIGNRINQPPDQGVKIIT |
| | | HKECNTIGINGMLFNTNKEGTLAFYTPDDITLNNSVALDPIDISI |
| | | ELNKLKSDLEELKEWIRRSNQKL |
| | | F2 (SEQ ID NO: 450): |
| | | QIDITKLQHVGVLVNSPKGMKISQNFETRYLILSLIPKIEDSNS |
| | | CGDQQIKQYKRLLDRLIIPLYDGLRLQKDVIVTNQESNENTDP |
| | | RTER |
| PIV3147 | S160C-V170C, | F1 (SEQ ID NO: 451): |
| | E209C-L234C, | FFGGVIGTIALGVATSAQITAAVALVEAKQARSDIEKLKEAIRD |
| | T277V, A463L, | TNKAVQCVQSSVGNLICAIKSVQDYVNKEIVPSIARLGCEAAG |
| | I474F | LQLGIALTQHYSCLTNIFGDNIGSLQEKGIKLQGIASCYRTNIT |
| | | EIFTTSTVDKYDIYDLLFTESIKVRVIDVDLNDYSIVLQVRLPLL |
| | | TRLLNTQIYKVDSISYNIQNREWYIPLPSHIMTKGAFLGGADV |
| | | KECIEAFSSYICPSDPGFVLNHEMESCLSGNISQCPRTTVTS |
| | | DIVPRYAFVNGGVVANCITTTCTCNGIGNRINQPPDQGVKIIT |
| | | HKECNTIGINGMLFNTNKEGTLAFYTPDDITLNNSVALDPIDISI |
| | | ELNKLKSDLEESKEWFRRSNQKL |
| | | F2 (SEQ ID NO: 452): |
| | | QIDITKLQHVGVLVNSPKGMKISQNFETRYLILSLIPKIEDSNS |
| | | CGDQQIKQYKRLLDRLIIPLYDGLRLQKDVIVTNQESNENTDP |
| | | RTER |
| PIV3148 | S160C-V170C, | F1 (SEQ ID NO: 453): |
| | E209C-S233C, | FFGGVIGTIALGVATSAQITAAVALVEAKQARSDIEKLKEAIRD |
| | T277V, A463L, | TNKAVQCVQSSVGNLICAIKSVQDYVNKEIVPSIARLGCEAAG |
| | S470L | LQLGIALTQHYSCLTNIFGDNIGSLQEKGIKLQGIACLYRTNIT |
| | | EIFTTSTVDKYDIYDLLFTESIKVRVIDVDLNDYSIVLQVRLPLL |
| | | TRLLNTQIYKVDSISYNIQNREWYIPLPSHIMTKGAFLGGADV |
| | | KECIEAFSSYICPSDPGFVLNHEMESCLSGNISQCPRTTVTS |
| | | DIVPRYAFVNGGVVANCITTTCTCNGIGNRINQPPDQGVKIIT |
| | | HKECNTIGINGMLFNTNKEGTLAFYTPDDITLNNSVALDPIDISI |
| | | ELNKLKSDLEELKEWIRRSNQKL |
| | | F2 (SEQ ID NO: 454): |
| | | QIDITKLQHVGVLVNSPKGMKISQNFETRYLILSLIPKIEDSNS |
| | | CGDQQIKQYKRLLDRLIIPLYDGLRLQKDVIVTNQESNENTDP |
| | | RTER |
| PIV3149 | S160C-V170C, | F1 (SEQ ID NO: 455): |
| | E209C-S233C, | FFGGVIGTIALGVATSAQITAAVALVEAKQARSDIEKLKEAIRD |
| | T277V, A463L, | TNKAVQCVQSSVGNLICAIKSVQDYVNKEIVPSIARLGCEAAG |
| | I474F | LQLGIALTQHYSCLTNIFGDNIGSLQEKGIKLQGIACLYRTNIT |
| | | EIFTTSTVDKYDIYDLLFTESIKVRVIDVDLNDYSIVLQVRLPLL |
| | | TRLLNTQIYKVDSISYNIQNREWYIPLPSHIMTKGAFLGGADV |
| | | KECIEAFSSYICPSDPGFVLNHEMESCLSGNISQCPRTTVTS |
| | | DIVPRYAFVNGGVVANCITTTCTCNGIGNRINQPPDQGVKIIT |
| | | HKECNTIGINGMLFNTNKEGTLAFYTPDDITLNNSVALDPIDISI |
| | | ELNKLKSDLEESKEWFRRSNQKL |
| | | F2 (SEQ ID NO: 456): |
| | | QIDITKLQHVGVLVNSPKGMKISQNFETRYLILSLIPKIEDSNS |
| | | CGDQQIKQYKRLLDRLIIPLYDGLRLQKDVIVTNQESNENTDP |
| | | RTER |
| PIV3151 | S160C-V170C, | F1 (SEQ ID NO: 457): |
| | G85C-E209C, | FFGGVIGTIALGVATSAQITAAVALVEAKQARSDIEKLKEAIRD |
| | T277V, A463L, | TNKAVQCVQSSVGNLICAIKSVQDYVNKEIVPSIARLGCEAAG |
| | I474F | LQLGIALTQHYSCLTNIFGDNIGSLQEKGIKLQGIASLYRTNITE |
| | | IFTTSTVDKYDIYDLLFTESIKVRVIDVDLNDYSIVLQVRLPLLT |
| | | RLLNTQIYKVDSISYNIQNREWYIPLPSHIMTKGAFLGGADVK |
| | | ECIEAFSSYICPSDPGFVLNHEMESCLSGNISQCPRTTVTSDI |
| | | VPRYAFVNGGVVANCITTTCTCNGIGNRINQPPDQGVKIITHK |
| | | ECNTIGINGMLFNTNKEGTLAFYTPDDITLNNSVALDPIDISIEL |
| | | NKLKSDLEESKEWFRRSNQKL |
| | | F2 (SEQ ID NO: 458): |
| | | QIDITKLQHVGVLVNSPKGMKISQNFETRYLILSLIPKIEDSNS |
| | | CGDQQIKQYKRLLDRLIIPLYDCLRLQKDVIVTNQESNENTDP |
| | | RTER |
| PIV3159 | S160C-V170C, | F1 (SEQ ID NO: 459): |
| | E209C-L234C, | FFGGVIGTIALGVATSAQITAAVALVEAKQARSDIEKLKEAIRD |
| | D455S, | TNKAVQCVQSSVGNLICAIKSVQDYVNKEIVPSIARLGCEAAG |
| | A463L, S470L | LQLGIALTQHYSCLTNIFGDNIGSLQEKGIKLQGIASCYRTNIT |
| | | EIFTTSTVDKYDIYDLLFTESIKVRVIDVDLNDYSITLQVRLPLL |
| | | TRLLNTQIYKVDSISYNIQNREWYIPLPSHIMTKGAFLGGADV |
| | | KECIEAFSSYICPSDPGFVLNHEMESCLSGNISQCPRTTVTS |
| | | DIVPRYAFVNGGVVANCITTTCTCNGIGNRINQPPDQGVKIIT |
| | | HKECNTIGINGMLFNTNKEGTLAFYTPDDITLNNSVALDPISISI |
| | | ELNKLKSDLEELKEWIRRSNQKL |
| | | F2 (SEQ ID NO: 460): |
| | | QIDITKLQHVGVLVNSPKGMKISQNFETRYLILSLIPKIEDSNS |
| | | CGDQQIKQYKRLLDRLIIPLYDGLRLQKDVIVTNQESNENTDP |
| | | RTER |
| PIV3160 | S160C-V170C, | F1 (SEQ ID NO: 461): |
| | E209C-S233C, | FFGGVIGTIALGVATSAQITAAVALVEAKQARSDIEKLKEAIRD |
| | D455S, | TNKAVQCVQSSVGNLICAIKSVQDYVNKEIVPSIARLGCEAAG |
| | A463L, S470L | LQLGIALTQHYSCLTNIFGDNIGSLQEKGIKLQGIACLYRTNIT |
| | | EIFTTSTVDKYDIYDLLFTESIKVRVIDVDLNDYSITLQVRLPLL |
| | | TRLLNTQIYKVDSISYNIQNREWYIPLPSHIMTKGAFLGGADV |
| | | KECIEAFSSYICPSDPGFVLNHEMESCLSGNISQCPRTTVTS |
| | | DIVPRYAFVNGGVVANCITTTCTCNGIGNRINQPPDQGVKIIT |
| | | HKECNTIGINGMLFNTNKEGTLAFYTPDDITLNNSVALDPISISI |
| | | ELNKLKSDLEELKEWIRRSNQKL |
| | | F2 (SEQ ID NO: 462): |
| | | QIDITKLQHVGVLVNSPKGMKISQNFETRYLILSLIPKIEDSNS |
| | | CGDQQIKQYKRLLDRLIIPLYDGLRLQKDVIVTNQESNENTDP |
| | | RTER |
| PIV3161 | S160C-V170C, | F1 (SEQ ID NO: 463): |
| | G85C-E209C, | FFGGVIGTIALGVATSAQITAAVALVEAKQARSDIEKLKEAIRD |
| | D455S, | TNKAVQCVQSSVGNLICAIKSVQDYVNKEIVPSIARLGCEAAG |
| | A463L, S470L | LQLGIALTQHYSCLTNIFGDNIGSLQEKGIKLQGIASLYRTNITE |
| | | IFTTSTVDKYDIYDLLFTESIKVRVIDVDLNDYSITLQVRLPLLT |
| | | RLLNTQIYKVDSISYNIQNREWYIPLPSHIMTKGAFLGGADVK |
| | | ECIEAFSSYICPSDPGFVLNHEMESCLSGNISQCPRTTVTSDI |
| | | VPRYAFVNGGVVANCITTTCTCNGIGNRINQPPDQGVKIITHK |
| | | ECNTIGINGMLFNTNKEGTLAFYTPDDITLNNSVALDPISISIEL |
| | | NKLKSDLEELKEWIRRSNQKL |
| | | F2 (SEQ ID NO: 464): |
| | | QIDITKLQHVGVLVNSPKGMKISQNFETRYLILSLIPKIEDSNS |
| | | CGDQQIKQYKRLLDRLIIPLYDCLRLQKDVIVTNQESNENTDP |
| | | RTER |
| PIV3162 | S160C-V170C, | F1 (SEQ ID NO: 465): |
| | E209C-L234C, | FFGGVIGTIALGVATSAQITAAVALVEAKQARSDIEKLKEAIRD |
| | T277V, | TNKAVQCVQSSVGNLICAIKSVQDYVNKEIVPSIARLGCEAAG |
| | D455S, | LQLGIALTQHYSCLTNIFGDNIGSLQEKGIKLQGIASCYRTNIT |
| | A463L, S470L | EIFTTSTVDKYDIYDLLFTESIKVRVIDVDLNDYSIVLQVRLPLL |
| | | TRLLNTQIYKVDSISYNIQNREWYIPLPSHIMTKGAFLGGADV |
| | | KECIEAFSSYICPSDPGFVLNHEMESCLSGNISQCPRTTVTS |
| | | DIVPRYAFVNGGVVANCITTTCTCNGIGNRINQPPDQGVKIIT |
| | | HKECNTIGINGMLFNTNKEGTLAFYTPDDITLNNSVALDPISISI |
| | | ELNKLKSDLEELKEWIRRSNQKL |
| | | F2 (SEQ ID NO: 466): |
| | | QIDITKLQHVGVLVNSPKGMKISQNFETRYLILSLIPKIEDSNS |
| | | CGDQQIKQYKRLLDRLIIPLYDGLRLQKDVIVTNQESNENTDP |
| | | RTER |
| PIV3163 | S160C-V170C, | F1 (SEQ ID NO: 467): |
| | E209C-S233C, | FFGGVIGTIALGVATSAQITAAVALVEAKQARSDIEKLKEAIRD |
| | T277V, | TNKAVQCVQSSVGNLICAIKSVQDYVNKEIVPSIARLGCEAAG |
| | D455S, | LQLGIALTQHYSCLTNIFGDNIGSLQEKGIKLQGIACLYRTNIT |
| | A463L, S470L | EIFTTSTVDKYDIYDLLFTESIKVRVIDVDLNDYSIVLQVRLPLL |
| | | TRLLNTQIYKVDSISYNIQNREWYIPLPSHIMTKGAFLGGADV |
| | | KECIEAFSSYICPSDPGFVLNHEMESCLSGNISQCPRTTVTS |
| | | DIVPRYAFVNGGVVANCITTTCTCNGIGNRINQPPDQGVKIIT |
| | | HKECNTIGINGMLFNTNKEGTLAFYTPDDITLNNSVALDPISISI |
| | | ELNKLKSDLEELKEWIRRSNQKL |
| | | F2 (SEQ ID NO: 468): |
| | | QIDITKLQHVGVLVNSPKGMKISQNFETRYLILSLIPKIEDSNS |
| | | CGDQQIKQYKRLLDRLIIPLYDGLRLQKDVIVTNQESNENTDP |
| | | RTER |
| PIV3164 | S160C-V170C, | F1 (SEQ ID NO: 469): |
| | G85C-E209C, | FFGGVIGTIALGVATSAQITAAVALVEAKQARSDIEKLKEAIRD |
| | T277V, | TNKAVQCVQSSVGNLICAIKSVQDYVNKEIVPSIARLGCEAAG |
| | D455S, | LQLGIALTQHYSCLTNIFGDNIGSLQEKGIKLQGIASLYRTNITE |
| | A463L, S470L | IFTTSTVDKYDIYDLLFTESIKVRVIDVDLNDYSIVLQVRLPLLT |
| | | RLLNTQIYKVDSISYNIQNREWYIPLPSHIMTKGAFLGGADVK |
| | | ECIEAFSSYICPSDPGFVLNHEMESCLSGNISQCPRTTVTSDI |
| | | VPRYAFVNGGVVANCITTTCTCNGIGNRINQPPDQGVKIITHK |
| | | ECNTIGINGMLFNTNKEGTLAFYTPDDITLNNSVALDPISISIEL |
| | | NKLKSDLEELKEWIRRSNQKL |
| | | F2 (SEQ ID NO: 470): |
| | | QIDITKLQHVGVLVNSPKGMKISQNFETRYLILSLIPKIEDSNS |
| | | CGDQQIKQYKRLLDRLIIPLYDCLRLQKDVIVTNQESNENTDP |
| | | RTER |
| PIV3191 | S160C-V170C, | F1 (SEQ ID NO: 471): |
| | A463L, S470L | FFGGVIGTIALGVATSAQITAAVALVEAKQARSDIEKLKEAIRD |
| | | TNKAVQCVQSSVGNLICAIKSVQDYVNKEIVPSIARLGCEAAG |
| | | LQLGIALTQHYSELTNIFGDNIGSLQEKGIKLQGIASLYRTNITE |
| | | IFTTSTVDKYDIYDLLFTESIKVRVIDVDLNDYSITLQVRLPLLT |
| | | RLLNTQIYKVDSISYNIQNREWYIPLPSHIMTKGAFLGGADVK |
| | | ECIEAFSSYICPSDPGFVLNHEMESCLSGNISQCPRTTVTSDI |
| | | VPRYAFVNGGVVANCITTTCTCNGIGNRINQPPDQGVKIITHK |
| | | ECNTIGINGMLFNTNKEGTLAFYTPDDITLNNSVALDPIDISIEL |
| | | NKLKSDLEELKEWIRRSNQKL |
| | | F2 (SEQ ID NO: 472): |
| | | QIDITKLQHVGVLVNSPKGMKISQNFETRYLILSLIPKIEDSNS |
| | | CGDQQIKQYKRLLDRLIIPLYDGLRLQKDVIVTNQESNENTDP |
| | | RTER |
| PIV3192 | S160C-V170C, | F1 (SEQ ID NO: 473): |
| | S470L | FFGGVIGTIALGVATSAQITAAVALVEAKQARSDIEKLKEAIRD |
| | | TNKAVQCVQSSVGNLICAIKSVQDYVNKEIVPSIARLGCEAAG |
| | | LQLGIALTQHYSELTNIFGDNIGSLQEKGIKLQGIASLYRTNITE |
| | | IFTTSTVDKYDIYDLLFTESIKVRVIDVDLNDYSITLQVRLPLLT |
| | | RLLNTQIYKVDSISYNIQNREWYIPLPSHIMTKGAFLGGADVK |
| | | ECIEAFSSYICPSDPGFVLNHEMESCLSGNISQCPRTTVTSDI |
| | | VPRYAFVNGGVVANCITTTCTCNGIGNRINQPPDQGVKIITHK |
| | | ECNTIGINGMLFNTNKEGTLAFYTPDDITLNNSVALDPIDISIEL |
| | | NKAKSDLEELKEWIRRSNQKL |
| | | F2 (SEQ ID NO: 474): |
| | | QIDITKLQHVGVLVNSPKGMKISQNFETRYLILSLIPKIEDSNS |
| | | CGDQQIKQYKRLLDRLIIPLYDGLRLQKDVIVTNQESNENTDP |
| | | RTER |

TABLE 67
Exemplary PIV3 F Protein Mutants Comprising Engineered Disulfide Mutations,
Cleavage Site Mutations, and Cavity Filling Mutations

| | | Amino Acid Sequence (residues 110-481 for F1 polypeptide |
|---|---|---|
| Mutant ID | Mutations | and residues 19-109 for F2 polypeptide) |
| PIV3156 | RTER(106-109) | F1 (SEQ ID NO: 475): |
| | → GSAS, | FFGGVIGTIALGVATSAQITAAVALVEAKQARSDIEKLKEAIR |
| | S160C-V170C, | DTNKAVQCVQSSVGNLICAIKSVQDYVNKEIVPSIARLGCE |
| | E209C-L234C, | AAGLQLGIALTQHYSCLTNIFGDNIGSLQEKGIKLQGIASCY |
| | A463L, S470L | RTNITEIFTTSTVDKYDIYDLLFTESIKVRVIDVDLNDYSITLQ |
| | | VRLPLLTRLLNTQIYKVDSISYNIQNREWYIPLPSHIMTKGA |
| | | FLGGADVKECIEAFSSYICPSDPGFVLNHEMESCLSGNISQ |
| | | CPRTTVTSDIVPRYAFVNGGVVANCITTTCTCNGIGNRINQ |
| | | PPDQGVKIITHKECNTIGINGMLFNTNKEGTLAFYTPDDITL |
| | | NNSVALDPIDISIELNKLKSDLEELKEWIRRSNQKL |
| | | F2 (SEQ ID NO: 476): |
| | | QIDITKLQHVGVLVNSPKGMKISQNFETRYLILSLIPKIEDSN |
| | | SCGDQQIKQYKRLLDRLIIPLYDGLRLQKDVIVTNQESNEN |
| | | TDPGSAS |
| PIV3157 | RTER(106-109) | F1 (SEQ ID NO: 477): |
| | → GSAS, | FFGGVIGTIALGVATSAQITAAVALVEAKQARSDIEKLKEAIR |
| | S160C-V170C, | DTNKAVQCVQSSVGNLICAIKSVQDYVNKEIVPSIARLGCE |
| | E209C-S233C, | AAGLQLGIALTQHYSCLTNIFGDNIGSLQEKGIKLQGIACLY |
| | A463L, S470L | RTNITEIFTTSTVDKYDIYDLLFTESIKVRVIDVDLNDYSITLQ |
| | | VRLPLLTRLLNTQIYKVDSISYNIQNREWYIPLPSHIMTKGA |
| | | FLGGADVKECIEAFSSYICPSDPGFVLNHEMESCLSGNISQ |
| | | CPRTTVTSDIVPRYAFVNGGVVANCITTTCTCNGIGNRINQ |
| | | PPDQGVKIITHKECNTIGINGMLFNTNKEGTLAFYTPDDITL |
| | | NNSVALDPIDISIELNKLKSDLEELKEWIRRSNQKL |
| | | F2 (SEQ ID NO: 478): |
| | | QIDITKLQHVGVLVNSPKGMKISQNFETRYLILSLIPKIEDSN |
| | | SCGDQQIKQYKRLLDRLIIPLYDGLRLQKDVIVTNQESNEN |
| | | TDPGSAS |
| PIV3158 | RTER(106-109) | F1 (SEQ ID NO: 479): |
| | → GSAS, | FFGGVIGTIALGVATSAQITAAVALVEAKQARSDIEKLKEAIR |
| | S160C-V170C, | DTNKAVQCVQSSVGNLICAIKSVQDYVNKEIVPSIARLGCE |
| | G85C-E209C, | AAGLQLGIALTQHYSCLTNIFGDNIGSLQEKGIKLQGIASLY |
| | A463L, S470L | RTNITEIFTTSTVDKYDIYDLLFTESIKVRVIDVDLNDYSITLQ |
| | | VRLPLLTRLLNTQIYKVDSISYNIQNREWYIPLPSHIMTKGA |
| | | FLGGADVKECIEAFSSYICPSDPGFVLNHEMESCLSGNISQ |
| | | CPRTTVTSDIVPRYAFVNGGVVANCITTTCTCNGIGNRINQ |
| | | PPDQGVKIITHKECNTIGINGMLFNTNKEGTLAFYTPDDITL |
| | | NNSVALDPIDISIELNKLKSDLEELKEWIRRSNQKL |
| | | F2 (SEQ ID NO: 480): |
| | | QIDITKLQHVGVLVNSPKGMKISQNFETRYLILSLIPKIEDSN |
| | | SCGDQQIKQYKRLLDRLIIPLYDCLRLQKDVIVTNQESNEN |
| | | TDPGSAS |
| PIV3165 | F110G, F111S, | F1 (SEQ ID NO: 481): |
| | S160C-V170C, | GSGGVIGTIALGVATSAQITAAVALVEAKQARSDIEKLKEAI |
| | E209C-L234C, | RDTNKAVQCVQSSVGNLICAIKSVQDYVNKEIVPSIARLGC |
| | A463L, S470L | EAAGLQLGIALTQHYSCLTNIFGDNIGSLQEKGIKLQGIASC |
| | | YRTNITEIFTTSTVDKYDIYDLLFTESIKVRVIDVDLNDYSITL |
| | | QVRLPLLTRLLNTQIYKVDSISYNIQNREWYIPLPSHIMTKG |
| | | AFLGGADVKECIEAFSSYICPSDPGFVLNHEMESCLSGNIS |
| | | QCPRTTVTSDIVPRYAFVNGGVVANCITTTCTCNGIGNRIN |
| | | QPPDQGVKIITHKECNTIGINGMLFNTNKEGTLAFYTPDDIT |
| | | LNNSVALDPIDISIELNKLKSDLEELKEWIRRSNQKL |
| | | F2 (SEQ ID NO: 482): |
| | | QIDITKLQHVGVLVNSPKGMKISQNFETRYLILSLIPKIEDSN |
| | | SCGDQQIKQYKRLLDRLIIPLYDGLRLQKDVIVTNQESNEN |
| | | TDPRTER |
| PIV3166 | F110G, F111S, | F1 (SEQ ID NO: 483): |
| | S160C-V170C, | GSGGVIGTIALGVATSAQITAAVALVEAKQARSDIEKLKEAI |
| | E209C-S233C, | RDTNKAVQCVQSSVGNLICAIKSVQDYVNKEIVPSIARLGC |
| | A463L, S470L | EAAGLQLGIALTQHYSCLTNIFGDNIGSLQEKGIKLQGIACL |
| | | YRTNITEIFTTSTVDKYDIYDLLFTESIKVRVIDVDLNDYSITL |
| | | QVRLPLLTRLLNTQIYKVDSISYNIQNREWYIPLPSHIMTKG |
| | | AFLGGADVKECIEAFSSYICPSDPGFVLNHEMESCLSGNIS |
| | | QCPRTTVTSDIVPRYAFVNGGVVANCITTTCTCNGIGNRIN |
| | | QPPDQGVKIITHKECNTIGINGMLFNTNKEGTLAFYTPDDIT |
| | | LNNSVALDPIDISIELNKLKSDLEELKEWIRRSNQKL |
| | | F2 (SEQ ID NO: 484): |
| | | QIDITKLQHVGVLVNSPKGMKISQNFETRYLILSLIPKIEDSN |
| | | SCGDQQIKQYKRLLDRLIIPLYDGLRLQKDVIVTNQESNEN |
| | | TDPRTER |
| PIV3189 | F110G, F111S, | F1 (SEQ ID NO: 485): |
| | S160C-V170C, | GSGGVIGTIALGVATSAQITAAVALVEAKQARSDIEKLKEAI |
| | A463L, S470L | RDTNKAVQCVQSSVGNLICAIKSVQDYVNKEIVPSIARLGC |
| | | EAAGLQLGIALTQHYSELTNIFGDNIGSLQEKGIKLQGIASL |
| | | YRTNITEIFTTSTVDKYDIYDLLFTESIKVRVIDVDLNDYSITL |
| | | QVRLPLLTRLLNTQIYKVDSISYNIQNREWYIPLPSHIMTKG |
| | | AFLGGADVKECIEAFSSYICPSDPGFVLNHEMESCLSGNIS |
| | | QCPRTTVTSDIVPRYAFVNGGVVANCITTTCTCNGIGNRIN |
| | | QPPDQGVKIITHKECNTIGINGMLFNTNKEGTLAFYTPDDIT |
| | | LNNSVALDPIDISIELNKLKSDLEELKEWIRRSNQKL |
| | | F2 (SEQ ID NO: 486): |
| | | QIDITKLQHVGVLVNSPKGMKISQNFETRYLILSLIPKIEDSN |
| | | SCGDQQIKQYKRLLDRLIIPLYDGLRLQKDVIVTNQESNEN |
| | | TDPRTER |
| PIV3190 | F110G, F111S, | F1 (SEQ ID NO: 487): |
| | S160C-V170C, | GSGGVIGTIALGVATSAQITAAVALVEAKQARSDIEKLKEAI |
| | S470L | RDTNKAVQCVQSSVGNLICAIKSVQDYVNKEIVPSIARLGC |
| | | EAAGLQLGIALTQHYSELTNIFGDNIGSLQEKGIKLQGIASL |
| | | YRTNITEIFTTSTVDKYDIYDLLFTESIKVRVIDVDLNDYSITL |
| | | QVRLPLLTRLLNTQIYKVDSISYNIQNREWYIPLPSHIMTKG |
| | | AFLGGADVKECIEAFSSYICPSDPGFVLNHEMESCLSGNIS |
| | | QCPRTTVTSDIVPRYAFVNGGVVANCITTTCTCNGIGNRIN |
| | | QPPDQGVKIITHKECNTIGINGMLFNTNKEGTLAFYTPDDIT |
| | | LNNSVALDPIDISIELNKAKSDLEELKEWIRRSNQKL |
| | | F2 (SEQ ID NO: 488): |
| | | QIDITKLQHVGVLVNSPKGMKISQNFETRYLILSLIPKIEDSN |
| | | SCGDQQIKQYKRLLDRLIIPLYDGLRLQKDVIVTNQESNEN |
| | | TDPRTER |

TABLE 68
Exemplary PIV3 F Protein Mutants Comprising Engineered Disulfide Mutations,
Cavity Filling Mutations, and Extended Ectodomain

| | | Amino Acid Sequence (residues 110-484 for F1 polypeptide |
|---|---|---|
| Mutant ID | Mutations | and residues 19-109 for F2 polypeptide) |
| PIV3167 | S160C-V170C, | F1 (SEQ ID NO: 489): |
| | A463L, S470L | FFGGVIGTIALGVATSAQITAAVALVEAKQARSDIEKLKEAIR |
| | | DTNKAVQCVQSSVGNLICAIKSVQDYVNKEIVPSIARLGCE |
| | | AAGLQLGIALTQHYSELTNIFGDNIGSLQEKGIKLQGIASLY |
| | | RTNITEIFTTSTVDKYDIYDLLFTESIKVRVIDVDLNDYSITLQ |
| | | VRLPLLTRLLNTQIYKVDSISYNIQNREWYIPLPSHIMTKGA |
| | | FLGGADVKECIEAFSSYICPSDPGFVLNHEMESCLSGNISQ |
| | | CPRTTVTSDIVPRYAFVNGGVVANCITTTCTCNGIGNRINQ |
| | | PPDQGVKIITHKECNTIGINGMLFNTNKEGTLAFYTPDDITL |
| | | NNSVALDPIDISIELNKLKSDLEELKEWIRRSNQKLDSI |
| | | F2 (SEQ ID NO: 490): |
| | | QIDITKLQHVGVLVNSPKGMKISQNFETRYLILSLIPKIEDSN |
| | | SCGDQQIKQYKRLLDRLIIPLYDGLRLQKDVIVTNQESNEN |
| | | TDPRTER |
| PIV3168 | S160C-V170C, | F1 (SEQ ID NO: 491): |
| | A463L, S477L | FFGGVIGTIALGVATSAQITAAVALVEAKQARSDIEKLKEAIR |
| | | DTNKAVQCVQSSVGNLICAIKSVQDYVNKEIVPSIARLGCE |
| | | AAGLQLGIALTQHYSELTNIFGDNIGSLQEKGIKLQGIASLY |
| | | RTNITEIFTTSTVDKYDIYDLLFTESIKVRVIDVDLNDYSITLQ |
| | | VRLPLLTRLLNTQIYKVDSISYNIQNREWYIPLPSHIMTKGA |
| | | FLGGADVKECIEAFSSYICPSDPGFVLNHEMESCLSGNISQ |
| | | CPRTTVTSDIVPRYAFVNGGVVANCITTTCTCNGIGNRINQ |
| | | PPDQGVKIITHKECNTIGINGMLFNTNKEGTLAFYTPDDITL |
| | | NNSVALDPIDISIELNKLKSDLEESKEWIRRLNQKLDSI |
| | | F2 (SEQ ID NO: 492): |
| | | QIDITKLQHVGVLVNSPKGMKISQNFETRYLILSLIPKIEDSN |
| | | SCGDQQIKQYKRLLDRLIIPLYDGLRLQKDVIVTNQESNEN |
| | | TDPRTER |
| PIV3187 | S160C-V170C, | F1 (SEQ ID NO: 493): |
| | E209C-L234C, | FFGGVIGTIALGVATSAQITAAVALVEAKQARSDIEKLKEAIR |
| | A463L, S470L | DTNKAVQCVQSSVGNLICAIKSVQDYVNKEIVPSIARLGCE |
| | | AAGLQLGIALTQHYSCLTNIFGDNIGSLQEKGIKLQGIASCY |
| | | RTNITEIFTTSTVDKYDIYDLLFTESIKVRVIDVDLNDYSITLQ |
| | | VRLPLLTRLLNTQIYKVDSISYNIQNREWYIPLPSHIMTKGA |
| | | FLGGADVKECIEAFSSYICPSDPGFVLNHEMESCLSGNISQ |
| | | CPRTTVTSDIVPRYAFVNGGVVANCITTTCTCNGIGNRINQ |
| | | PPDQGVKIITHKECNTIGINGMLFNTNKEGTLAFYTPDDITL |
| | | NNSVALDPIDISIELNKLKSDLEELKEWIRRSNQKLDSI |
| | | F2 (SEQ ID NO: 494): |
| | | QIDITKLQHVGVLVNSPKGMKISQNFETRYLILSLIPKIEDSN |
| | | SCGDQQIKQYKRLLDRLIIPLYDGLRLQKDVIVTNQESNEN |
| | | TDPRTER |
| PIV3193 | S160C-V170C, | F1 (SEQ ID NO: 495): |
| | S470L | FFGGVIGTIALGVATSAQITAAVALVEAKQARSDIEKLKEAIR |
| | | DTNKAVQCVQSSVGNLICAIKSVQDYVNKEIVPSIARLGCE |
| | | AAGLQLGIALTQHYSELTNIFGDNIGSLQEKGIKLQGIASLY |
| | | RTNITEIFTTSTVDKYDIYDLLFTESIKVRVIDVDLNDYSITLQ |
| | | VRLPLLTRLLNTQIYKVDSISYNIQNREWYIPLPSHIMTKGA |
| | | FLGGADVKECIEAFSSYICPSDPGFVLNHEMESCLSGNISQ |
| | | CPRTTVTSDIVPRYAFVNGGVVANCITTTCTCNGIGNRINQ |
| | | PPDQGVKIITHKECNTIGINGMLFNTNKEGTLAFYTPDDITL |
| | | NNSVALDPIDISIELNKAKSDLEELKEWIRRSNQKLDSI |
| | | F2 (SEQ ID NO: 496): |
| | | QIDITKLQHVGVLVNSPKGMKISQNFETRYLILSLIPKIEDSN |
| | | SCGDQQIKQYKRLLDRLIIPLYDGLRLQKDVIVTNQESNEN |
| | | TDPRTER |

TABLE 69
Exemplary PIV3 F Protein Mutants Comprising Engineered Disulfide Mutations,
Cleavage Site Mutations, Cavity Filling Mutations, and Extended Ectodomain

| | | Amino Acid Sequence (residues 110-484 for F1 polypeptide |
|---|---|---|
| Mutant ID | Mutations | and residues 19-109 for F2 polypeptide) |
| PIV3186 | F110G, F111S, | F1 (SEQ ID NO: 497): |
| | S160C-V170C, | GSGGVIGTIALGVATSAQITAAVALVEAKQARSDIEKLKEAI |
| | A463L, S470L | RDTNKAVQCVQSSVGNLICAIKSVQDYVNKEIVPSIARLGC |
| | | EAAGLQLGIALTQHYSELTNIFGDNIGSLQEKGIKLQGIASL |
| | | YRTNITEIFTTSTVDKYDIYDLLFTESIKVRVIDVDLNDYSITL |
| | | QVRLPLLTRLLNTQIYKVDSISYNIQNREWYIPLPSHIMTKG |
| | | AFLGGADVKECIEAFSSYICPSDPGFVLNHEMESCLSGNIS |
| | | QCPRTTVTSDIVPRYAFVNGGVVANCITTTCTCNGIGNRIN |
| | | QPPDQGVKIITHKECNTIGINGMLFNTNKEGTLAFYTPDDIT |
| | | LNNSVALDPIDISIELNKLKSDLEELKEWIRRSNQKLDSI |
| | | F2 (SEQ ID NO: 498): |
| | | QIDITKLQHVGVLVNSPKGMKISQNFETRYLILSLIPKIEDSN |
| | | SCGDQQIKQYKRLLDRLIIPLYDGLRLQKDVIVTNQESNEN |
| | | TDPRTER |
| PIV3188 | F110G, F111S, | F1 (SEQ ID NO: 499): |
| | S160C-V170C, | GSGGVIGTIALGVATSAQITAAVALVEAKQARSDIEKLKEAI |
| | E209C-L234C, | RDTNKAVQCVQSSVGNLICAIKSVQDYVNKEIVPSIARLGC |
| | A463L, S470L | EAAGLQLGIALTQHYSCLTNIFGDNIGSLQEKGIKLQGIASC |
| | | YRTNITEIFTTSTVDKYDIYDLLFTESIKVRVIDVDLNDYSITL |
| | | QVRLPLLTRLLNTQIYKVDSISYNIQNREWYIPLPSHIMTKG |
| | | AFLGGADVKECIEAFSSYICPSDPGFVLNHEMESCLSGNIS |
| | | QCPRTTVTSDIVPRYAFVNGGVVANCITTTCTCNGIGNRIN |
| | | QPPDQGVKIITHKECNTIGINGMLFNTNKEGTLAFYTPDDIT |
| | | LNNSVALDPIDISIELNKLKSDLEELKEWIRRSNQKLDSI |
| | | F2 (SEQ ID NO: 500): |
| | | QIDITKLQHVGVLVNSPKGMKISQNFETRYLILSLIPKIEDSN |
| | | SCGDQQIKQYKRLLDRLIIPLYDGLRLQKDVIVTNQESNEN |
| | | TDPRTER |
| PIV3194 | F110G, F111S, | F1 (SEQ ID NO: 501): |
| | S160C-V170C, | GSGGVIGTIALGVATSAQITAAVALVEAKQARSDIEKLKEAI |
| | S470L | RDTNKAVQCVQSSVGNLICAIKSVQDYVNKEIVPSIARLGC |
| | | EAAGLQLGIALTQHYSELTNIFGDNIGSLQEKGIKLQGIASL |
| | | YRTNITEIFTTSTVDKYDIYDLLFTESIKVRVIDVDLNDYSITL |
| | | QVRLPLLTRLLNTQIYKVDSISYNIQNREWYIPLPSHIMTKG |
| | | AFLGGADVKECIEAFSSYICPSDPGFVLNHEMESCLSGNIS |
| | | QCPRTTVTSDIVPRYAFVNGGVVANCITTTCTCNGIGNRIN |
| | | QPPDQGVKIITHKECNTIGINGMLFNTNKEGTLAFYTPDDIT |
| | | LNNSVALDPIDISIELNKAKSDLEELKEWIRRSNQKLDSI |
| | | F2 (SEQ ID NO: 502): |
| | | QIDITKLQHVGVLVNSPKGMKISQNFETRYLILSLIPKIEDSN |
| | | SCGDQQIKQYKRLLDRLIIPLYDGLRLQKDVIVTNQESNEN |
| | | TDPRTER |

### Example 28. Additional PIV3 F Protein Mutant Expression Vector Construction

A nucleic acid molecule encoding the consensus PIV3 F0 polypeptide set forth in SEQ ID NO:305 or 420 was mutated using standard molecular biology techniques to encode a precursor polypeptide for a PIV3 F mutant having the introduced amino acid mutations disclosed in Example 27. The structure and components of the precursor polypeptide are set forth in FIG. 1 and SEQ ID NO:305 or 420. The precursor polypeptide of mutants set forth in SEQ ID NO:305 comprises residues as described in Example 14. For mutants set forth in SEQ ID NO:420, the precursor polypeptide comprises identical residues 1-109 and 482-534 set forth in SEQ ID:305, with the exemption of a longer F1 polypeptide (residues 110-484).

The cloning of PIV3 F protein mutants in expression vector and the preparation of the plasmid DNA for transfection were performed as described in Example 14.

### Example 29. Expression and Conformational Integrity of Additional PIV3 F Protein Mutants

Transient transfection of the PIV3 F protein mutants and preparation of crude cell supernatants were performed as described in Example 15.

The OCTET HTX (Sartorius, Gottingen, Germany) instrument was used to evaluate the expression and conformational integrity for each mutant. All measurements were conducted at 30° C. temperature in 96-well black plates (Corning, Corning, NY) with a final volume of 240 μL per well at a constant agitation rate of 1000 RPM.

### 29A: Quantitation of Expression of PIV3 F Protein Mutants

Crude cell supernatant was used to quantitate the expression levels of PIV3 F protein mutants. The protein expression of mutants was measured using the same procedure as described in Example 16A. The results for single and combination mutants are presented in Tables 70, 71, 72, and 73. More than two-fold increase in protein expression than WT control was observed in 4 out of 8 single mutants, PIV3136, PIV3138, PIV3119 and PIV3152 (Table 70). In addition, 20 out of 31 combination mutants showed more than a 4-fold increase in protein expression compared with the WT control (Tables 71 and 73).

### 29B: Conformational Integrity of PIV3 F Protein Mutants

Conformational integrity of PIV3 F protein mutants was evaluated by a thermal stress experiment. Crude cell culture supernatants were normalized based on the results of the titer quantitation and then incubated for 1 hour at room temperature, 53° C., and 56° C. The thermal-stress assay and analysis were performed as described in Example 16B. The reactivities of the single and combination mutants to PIA174 mAb are presented in Tables 70, 71, 72 and 73. After thermal stress at 56° C., PIV3134 and PIV3138 were the only single mutants retaining higher reactivity to PIA174 mAb compared with WT control (Table 70). In addition, 14 out of 31 combination mutants retained much higher reactivity to PIA174 mAb compared with WT control after thermal stress at 56° C. (Tables 71 and 73). Two additional mutants, PIV3167 and PIV3168, were evaluated in a separate experiment, which included mutant PIV3165 as an internal control. While PIV3165 showed similar trend in thermal stability as shown in Table 71, both PIV3167 and PIV3168 also showed comparable reactivity to PIA174 mAb as PIV3165 after thermal stress at 56° C. (Table 72).

TABLE 70
Protein expression and thermal stability of PIV3 F protein mutants.

| | | Response Shift with PIA174 mAb | | |
|---|---|---|---|---|
| | Protein | | 53° C. stress | 56° C. stress |
| | expression | No stress | resistance | resistance |
| Mutant ID | (mg/L) | (nm) | (nm) | (nm) |
| WT control | 5.3 | 0.536 | 0.434 | 0.410 |
| PIV3134 | 5.8 | 0.842 | 0.764 | 0.758 |
| PIV3135 | 4.7 | 0.729 | 0.028 | 0.055 |
| PIV3136 | 10.0 | 0.018 | 0.013 | 0.008 |
| PIV3137 | 2.6 | 0.027 | 0.013 | −0.008 |
| PIV3138 | 11.7 | 1.451 | 1.416 | 0.651 |
| PIV3139 | 17.4 | 1.456 | 0.918 | −0.006 |
| PIV3152 | 11.7 | 1.565 | 1.382 | 0.197 |

TABLE 71
Protein expression and thermal stability of PIV3 F protein mutants
comprising engineered disulfide mutations, cleavage site mutations,
extended ectodomain, alternative trimerization domain, electrostatic
mutations, and cavity filling mutations.

| | | Response Shift with PIA174 mAb | | |
|---|---|---|---|---|
| | Protein | | 53° C. stress | 56° C. stress |
| | expression | No stress | resistance | resistance |
| Mutant ID | (mg/L) | (nm) | (nm) | (nm) |
| WT Control | 5.3 | 0.536 | 0.434 | 0.410 |
| PIV3140 | 22.2 | 1.618 | 1.479 | 0.857 |
| PIV3141 | 28.3 | 1.533 | 1.415 | 0.074 |
| PIV3142 | 24.2 | 1.234 | 0.252 | −0.001 |
| PIV3143 | 28.4 | 1.306 | 0.064 | 0.010 |
| PIV3144 | 3.6 | 0.967 | 0.866 | 0.335 |
| PIV3145 | 8.1 | 1.207 | 1.159 | 0.151 |
| PIV3146 | 37.0 | 1.579 | 1.491 | 0.971 |
| PIV3147 | 45.7 | 1.457 | 1.372 | 0.044 |
| PIV3148 | 33.2 | 1.232 | 0.348 | 0.015 |
| PIV3149 | 22.2 | 1.447 | 0.180 | −0.024 |
| PIV3151 | 9.6 | 1.097 | 1.027 | 0.178 |
| PIV3156 | 54.3 | 1.175 | 1.169 | 0.587 |
| PIV3157 | 14.7 | 1.303 | 0.711 | −0.007 |
| PIV3158 | 4.7 | 1.088 | 0.981 | 0.550 |
| PIV3159 | 17.8 | 1.597 | 1.438 | 0.546 |
| PIV3160 | 6.8 | 1.205 | 0.226 | 0.092 |
| PIV3161 | 4.9 | 0.880 | 0.637 | 0.158 |
| PIV3162 | 51.6 | 1.295 | 1.174 | 0.361 |
| PIV3163 | 5.8 | 1.129 | 0.169 | 0.014 |
| PIV3164 | 2.9 | 0.604 | 0.420 | 0.140 |
| PIV3165 | 23.0 | 1.554 | 1.452 | 1.343 |
| PIV3166 | 17.8 | 1.219 | 0.824 | 0.092 |

TABLE 72
Protein expression and thermal stability of PIV3 F protein
mutants comprising engineered disulfide mutations, extended
ectodomain, and cavity filling mutations.

| | | Response Shift with PIA174 mAb | | |
|---|---|---|---|---|
| | Protein | | 53° C. stress | 56° C. stress |
| | expression | No stress | resistance | resistance |
| Mutant ID | (mg/L) | (nm) | (nm) | (nm) |
| PIV3165 | 92 | 1.108 | 0.938 | 0.814 |
| PIV3167 | 30 | 1.275 | 1.025 | 0.617 |
| PIV3168 | 76 | 1.299 | 1.048 | 0.800 |

TABLE 73
Protein expression and thermal stability of PIV3 F protein mutants
comprising engineered disulfide mutations, cleavage site mutations,
extended ectodomain, and cavity filling mutations.

| | | Response Shift with PIA174 mAb | | |
|---|---|---|---|---|
| | Protein | | 53° C. stress | 56° C. stress |
| | expression | No stress | resistance | resistance |
| Mutant ID | (mg/L) | (nm) | (nm) | (nm) |
| WT Control | 5.6 | 0.552 | 0.409 | 0.360 |
| PIV3186 | 27.6 | 1.204 | 1.097 | 0.980 |
| PIV3187 | 35.6 | 1.361 | 1.257 | 0.968 |
| PIV3188 | 45.0 | 1.260 | 1.206 | 1.106 |
| PIV3189 | 83.4 | 1.262 | 1.187 | 0.982 |
| PIV3190 | 78.6 | 1.091 | 1.158 | 0.964 |
| PIV3191 | 30.5 | 1.302 | 1.325 | 0.909 |
| PIV3192 | 49.9 | 1.260 | 1.228 | 0.983 |
| PIV3193 | 30.1 | 1.432 | 1.358 | 1.160 |
| PIV3194 | 46.0 | 1.278 | 1.262 | 1.065 |

### Example 30: Additional Stabilized PIV3 F Protein Mutants in Prefusion Conformation as Protein Subunit Elicit Neutralizing Responses in Mice

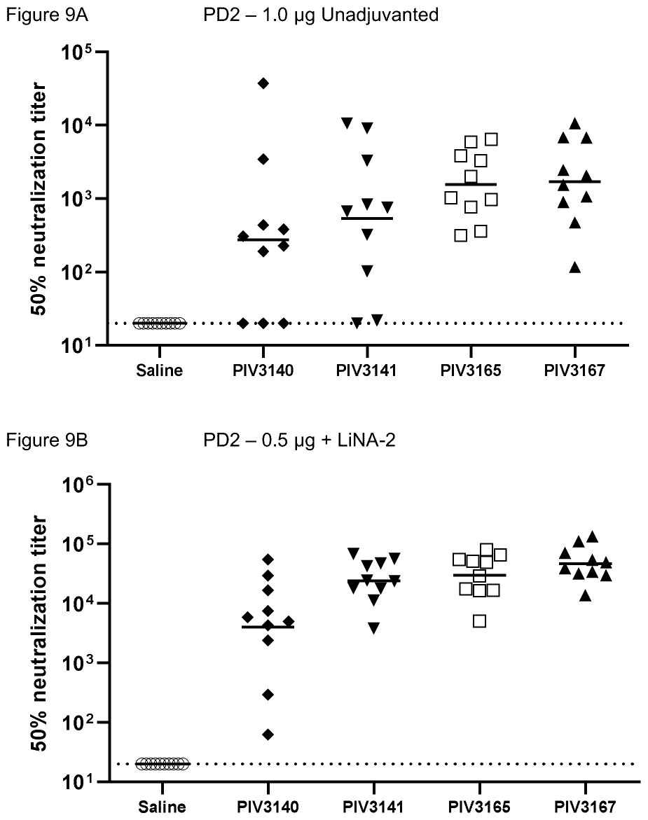

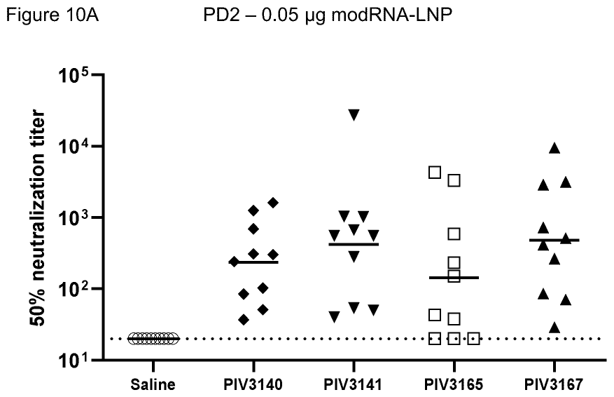

Protein subunit antigens were prepared as described in Example 18 above. Female Balb/c mice were immunized with either 1.0 μg or 0.5 μg of protein subunit with LiNA2 (20 μg MPLA, 10 μg QS-21 per dose) as adjuvant, of either PIV3 F protein mutants PIV3140, PIV3141, PIV3165, or PIV3167. Immunizations were given intramuscularly at weeks 0 and 3 (Table 74). Post-dose 1 (PD1, week 3) and post-dose 2 (PD2, week 5) sera were evaluated in a PIV3 neutralization assay, and the results are reported as described in Example 23. Overall, all combination mutants tested elicited a neutralizing response following two immunizations in mice (Table 75, FIG. 9). At 0.5 μg dose with adjuvant, PIV3141, PIV3165 and PIV3167 showed the highest neutralizing titers. At 1.0 μg without adjuvant, PIV3165 and PIV3167 showed higher neutralizing titers than the other two mutants.

TABLE 74
Immunization schedule of the murine immunogenicity
study comparing PIV3 F protein mutants.

| | |
|---|---|
| PIV3 F protein mutant dose | 1.0 μg, 0.5 ug of protein with LiNA-2 |
| Vaccination | Weeks 0, 3 |
| Bleed | Weeks 3 (PD1) |
| | 5 (PD2) |

TABLE 75
Geometric mean and fold-rise of 50% neutralizing titers of Balb/c
mice following two immunizations with PIV3 F protein mutants.

| | |
|---|---|
| 1.0 μg Unadjuvanted | 0.5 μg + LiNA-2 |

| Mutant ID | GMT | Fold rise | GMT | Fold rise |
|---|---|---|---|---|
| PIV3140 | 273 | 13.7 | 4015 | 200.8 |
| PIV3141 | 535 | 26.8 | 23909 | 1195.5 |
| PIV3165 | 1557 | 77.9 | 29592 | 1479.6 |
| PIV3167 | 1704 | 85.2 | 46732 | 2336.6 |
| Saline | 20 | 1.0 | 20 | 1.0 |

### Example 31: ADDITIONAL PREFUSION-STABILIZED, FULL-LENGTH PIV3 F PROTEIN Mutants Expressed Through modRNA Elicit Neutralizing Responses in Mice

Formulated modRNA-LNPs were prepared as described in Examples 19 and 20 above. Two lower dose levels were selected in order to provide more sensitive results and potentially differentiate different designs. Female Balb/c mice were immunized with 0.05 μg or 0.2 μg of LNP-formulated modRNA encoding full length PIV3 F protein mutants PIV3031, PIV3135, PIV3138, PIV3140, PIV3141, PIV3165, or PIV3167. Immunizations were given intramuscularly at weeks 0 and 3 (Table 76). Post-dose 1 (PD1, week 3) and post-dose 2 (PD2, week 5) sera were evaluated in a PIV3 neutralization assay, and the results are reported as described in Example 24 (Table 77).

TABLE 76
Immunization schedule of the murine immunogenicity study comparing
full-length PIV3 F protein mutants encoded from modRNA.

| | |
|---|---|
| PIV3 F protein mutant modRNA dose | 0.05 μg, 0.2 μg modRNA-LNP |
| Vaccination | Weeks 0, 3 |
| Bleed | Weeks 3 (PD1) |
| | 5 (PD2) |

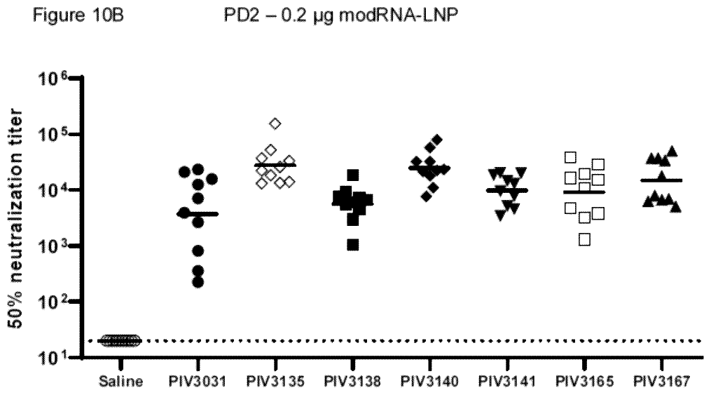

Overall, all combination mutants tested elicited a neutralizing response following two immunizations in mice (Table 77, FIG. 10). At 0.05 μg modRNA-LNP dose, PIV3141 and PIV3167 showed higher neutralizing titers than other tested mutants. At 0.2 μg modRNA-LNP dose, PIV3135 and PIV3140 elicited higher neutralizing titers than other tested mutants.

TABLE 77
Geometric mean and fold-rise of 50% neutralizing
titers of Balb/c mice following two immunizations
with modRNA-LIP of PIV3 F mutants.

| | 0.05 μg modRNA-LNP | | 0.2 μg modRNA-LNP | |
|---|---|---|---|---|
| Mutant ID | GMT | Fold rise | GMT | Fold rise |
| PIV3031 | ND | ND | 3694 | 184.7 |
| PIV3135 | ND | ND | 27695 | 1384.8 |
| PIV3138 | ND | ND | 5600 | 280.0 |
| PIV3140 | 235 | 11.8 | 24683 | 1234.2 |
| PIV3141 | 422 | 21.1 | 9881 | 494.1 |
| PIV3165 | 144 | 7.2 | 9249 | 462.5 |
| PIV3167 | 480 | 24.0 | 14792 | 739.6 |
| Saline | 20 | 1.0 | 20 | 1.0 |

“ND”—Not Determined.

### Example 32: In Vitro Expression (IVE) Imaging Assay for Hmpv B Antigen Screening

Additional modRNA encoding hMPV B antigens were prepared as described in Example 19 above. To characterize hMPVB antigens encoded from modRNA, an in vitro expression (IVE) imaging assay was performed in HeLa cells. Cells were plated in 384 well PDL coated imaging plates (PerkinElmer) and transfected with modRNAs formulated with Lipofectamine MessengerMAX (Thermo Fisher Scientific). ModRNAs encoded full-length wild type (WT) hMPV B F protein or full-length hMPV B F protein mutants hMPV170, hMPV171, hMPV172, hMPV173, hMPV174, hMPV175, hMPV176 and hMPV177. ModRNAs were diluted in Opti-MEM (Thermo Fisher Scientific) media to create an 11 point 2-fold dilution series for each construct. Expression of hMPVB F protein was examined at 22 hours post transfection by immunofluorescence imaging using hMPV-2 mAb, which specifically binds hMPV F in its prefusion form. To image the plate, cells were fixed with 4% paraformaldehyde, washed, and blocked with 6% BSA (Fraction V). Subsequently, plates were incubated with hMPV-2 mAb at 0.4 μg/ml in DPBS containing 6% BSA overnight at 4° C., followed by DPBS wash and anti-human AlexaFluor-488 labeled secondary antibody (0.2 μg/ml) incubation for 2 hours at RT. Hoechst nuclear stain is included at 0.2 μg/ml to allow cell count. The plates were subjected to final washes by DPBS to remove excess secondary antibody before imaging on the Opera Phenix High Content Imager. The images were analyzed with Signals Image Artist software and multiple endpoints were calculated, including MFI (mean fluorescence intensity), cell count (as a measure of toxicity/cell death) and % hMPVB-F positive cells. For the percent hMPVB-F positive cells readout, WT hMPVB-F modRNA at 25 ng/well was used as the 100% control and Lipofectamine MessengerMax alone without modRNA was used as the negative control. EC₅₀ curves were generated using Signals GeneData Screener software. Mean EC₅₀ values of the percent hMPVB-F positive cells readout reported in Table 78 below were used for hMPVB-F antigen selection. Several mutant constructs showed a trend of improved EC50 compared to WT.

TABLE 78
EC50 of hMPVB antigens IVE in Hela measured by hMPV-2 mab

| | | Mean EC₅₀ | | |
|---|---|---|---|---|
| Antigen ID | Mutation | (ng/ml) | SD | Ns |
| hMPV163 | WT | 2.62 | 0.604 | 9 |
| hMPV170 | L66P | 1.75 | 0.912 | 3 |
| hMPV171 | L187P | 1.08 | 0.378 | 3 |
| hMPV172 | L187P, Q100R, S101R | 1.68 | 0.249 | 3 |
| hMPV173 | A140C, S149C, L187P | 3.28 | 0.174 | 3 |
| hMPV174 | A140C, S149C, L187P, | 4.02 | 0.890 | 3 |
| | Q100R, S101R | | | |
| hMPV175 | T49I | 2.16 | 0.416 | 3 |
| hMPV176 | T365I | 2.30 | 0.536 | 3 |
| hMPV177 | T49I, T365I | 1.35 | 0.705 | 3 |

Ns: number of independent IVE experiments.

### Example 33: Generation of hMPV and PIV1 Antibodies

Human Monoclonal Antibodies Derived from B-cells were isolated from peripheral blood mononuclear cells (PBMCs) of healthy adults.

PIV1 and hMPV F protein in pre- and postfusion conformation (1 mg) were brought to PBS pH 7.2 with Zebra™ spin desalting columns (ThermoFisher), followed by incubation with 20-fold molar excess (13 μL) of 10 mM fresh prepared EZ-Link™ Sulfo-NHS-LC-LC-Biotin (ThermoFisher) for 2 hours on ice. The 20-fold molar excess biotin was calculated to achieve labelling of 4-6 biotins per molecule while occupying less than 10% of lysine of full protein. This aims to avoid blocking of potential antibody epitopes. The excess of non-reacted and hydrolyzed biotin reagent was removed with Zeba™ spin desalting columns. The confirmation of biotinylation was detected by Octet HTX instrument (ForteBio/Sartorius). Briefly, Streptavidin biosensors were used to detect biotinylated protein. Following establishment of a baseline signal in the appropriate matrix (PBS, 1% BSA pH 7.2) for 1 min, 3.6 μg biotinylated F-protein were loaded for 5 min onto the biosensors for detection. BLI data acquisition and analyses were accomplished with software version 12.2.

Flow cytometry was used to isolate antigen (fusion protein F) specific B cells. Briefly, PBMCs from vaccinated subjects were first incubated with fixable viability dye eFluor™ 780 (Invitrogen) in 1% BSA/PBS at 4° C. for 20 minutes to exclude dead cells. Cells were then washed with 1% BSA/PBS to remove the eFluor™ 780, and then centrifuged at 500×g, 5 minutes. The PBMCs were then incubated with 50 nM of biotinylated F protein at 4° C. for 1 hour, followed by washing 3 times with 1% BSA/PBS. A secondary antibody cocktail comprised of anti-CD19-BV421 (Biolegend), anti-human IgG-AF488 (H+L) (Jackson Immunoresearch) or anti-human IgG (Hc)-AF488 (BD Bioscience), Streptavidin-AF647 and Streptavidin-PE (Jackson Immunoresearch) was used to identify the antigen-reactive memory B cells. FACS sorting was carried out with a BD FACSAria Fusion or a FACSAria II sorting machine. A 2-step sorting strategy was applied for higher efficiency and better resolution. The first step of high-speed sorting was run at a speed of around 15,000 events/second. The sample tube was kept at 4° C. during sorting. The antigen-reactive B cells were gated as CD19+ IgG+ Antigen+ and sorted into 1.5 mL Eppendorf tube with 1% BSA/PBS. The sorted cells were loaded for a second-step single cell sort with a low speed of 10-20 events/second. The antigen-reactive single B cells were then sorted into 96-well plates containing 20 μL of lysis buffer (5 μL of 5× SuperScript IV reverse transcriptase buffer, 0.5 μL of RNase inhibitor [New England Biolab], 1.25 μL of 0.1 M DTT [Invitrogen], 0.625 μL of 10% NP40 [ThermoFisher]). Plates were then snap-frozen on dry ice and stored at −80° C. until needed.

Antibody heavy and light chain variable region cDNA were amplified from the sorted single B cells by reverse transcription and nested PCR, then cloned into linear pTT5 expression vectors to produce complete IgG1 antibodies.

Paired plasmids containing heavy chain (Hc) and light chain (Lc) from resulting colonies of transformed E. coli 10G competent cells (Lucigen) were amplified with a Templiphi DNA amplification kit (Cytiva). Briefly, individual colonies were picked and lysed in 5 μL sample buffer at 95° C. for 3 minutes. Then 5 μL of enzyme master mix was added and the reaction was incubated at 30° C. for 16-18 hours. Deactivation of the enzyme was accomplished by incubation for 10 minutes at 65° C. The resulting Hc and Lc DNA were co-transfected into Expi293F cells grown in 2 mL 96-deep well plates with ExpiFectamine™ 293 transfection kit (Thermo Fisher). Each well of the 96-deep well containing around 1.5×10⁶ cells in 500 μL. A total of 0.5 μg of Hc and Lc DNA (1:2 or 1:3 ration) was diluted in 25 μL OptiMEM medium, separately 1.35 μL of ExpiFectamine™ 293 was diluted in 25 μL OptiMEM medium for each well. After 5 minutes incubation, the diluted ExpiFectamine™ 293 and the diluted DNA were mixed and incubated for 20-25 minutes. The complex was added to the Expi293F cells and cultured overnight. Eighteen-22 hours after transfection, 2.5 μL Enhancer 1 (1:200 of total volume) and 25 μL enhancer 2 (1:20 of total volume) was added to each well. Transfected cells in 96-deep well were cultured on an orbital shaker (3 mm orbit diameter) at 1000 rpm at 37° C. with 8% CO2 humidity. The transfection product was harvested on day 5 after transfection.

### Example 34: ANTIBODY PRODUCTION AND PURIFICATION

Heavy chain and light chain plasmids were purified with Qiagen Plasmid Midi Plus kit (Qiagen) and transfected into Expi293F cells with ExpiFectamine 293 transfection kit (ThermoFisher) in a 1:2 ratio. For 50 mL expression, a total of 50 μg of heavy chain and light chain plasmids were diluted in 2.5 mL OptiMEM medium, and 135 uL of ExpiFectamine 293 Reagent was diluted in 2.5 mL OptiMEM medium, then the DNA and ExpiFectamine 293 Reagent was mixed and incubated at room temperature for approximately 20 minutes. After incubation, the transfection mix was added to 42.5 mL of Expi293F cells and cultured at 165 rpm at 37° C. with 8% CO2 humidity. Approximately 18-22h post-transfection, 250 μL ExpiFectamine Enhancer 1 plus 2.5 mL Enhancer 2 were added to the transfection culture flasks. The supernatant was collected on the 5^{th} day of transfection and followed by determination of IgG concentration by BioLayer Interferometry using Protein A biosensors (Fortebio/Sartorius). For larger volume of transfection, the transfection reaction was scaled up based on the 50 mL transfection.

Expressed antibodies from 50 mL culture were purified by Magnetic Protein A beads (Promega). For transfection volumes larger than 100 mL, antibodies were purified by affinity chromatography using a pre-packed 5 mL HiTrap Protein A column or a XK20-50 (Cytiva, US)) column shell manually packed with Protein A resin (Cytiva, US)G). The size of the purification column was determined based on the amount of antibody in the supernatants and the approximate 20 mg/mL resin binding capacity of human IgG. The HiTrap Protein A was equilibrated with at least 5 column volumes (CV) PBS buffer at 4 mL/mL on an AEKTA Avant chromatography system (GE Healthcare, PA). Antibody application was done with a reduced flow rate at 1.5-2 mL/min. Bound antibody was eluted with 0.2M glycine, pH3.5 with collection of 2 mL fractions. To neutralize the low pH after elution, 0.75 mL of 1M Tris-Cl pH 9.0 was added to each collection tube prior to sample collection.

XK20-50 (GE Healthcare, PA) column was packed and equilibrated with three column volumes of PBS buffer and run at 8 mL/min on an AKTA Avant chromatography system (GE Healthcare, PA). Bound antibody was eluted with 0.2M glycine, pH 3 with collection of 25 mL fractions. To neutralize the low pH after elution, 4 mL of 1 M Tris-Cl pH 9.0 was added to each collection tube prior to sample collection. Peak fractions were analyzed by SDS-PAGE with Coomassie staining in reducing and non-reducing conditions.

### 34 AVerification of Purified Antibody by SDS-PAGE Followed by Coomassie Staining

SDS-PAGE was performed with the Novex Mini-Cell electrophoresis system (Life Technologies/ThermoFisher, NJ) using precast 4-12% Bis-Tris gels and NuPAGE MES SDS Running Buffer. Samples were analyzed using non-reducing and reducing (with 100 mM dithiothreitol) conditions and visualized using SimplyBlue SafeStain (Life Technologies/ThermoFisher, NJ) according to the manufacturer's protocol. The purity of IgG was estimated by image analysis of the destained gels using a GS-900 Calibrated Densitometer (BioRAD,CA) with ImageQuant 5.2.1 software.

### 34B Determination of Purity by Absorbance

IgG concentration was determined by absorbance at 280 nm measured with a NanoDrop 8000 spectrophotometer (ThermoScientific, NJ) according to the manufacturer's instructions. The extinction coefficient used for IgG was 1.37, which is the A280 absorbance of a 1 mg/mL IgG solution. The formula used for calculation of IgG concentration was A280/1.37=Concentration (mg/mL)

### Example 35: Determination of HPVI1-8 and HMPV-2 MABS Binding of Protein by Biolayer Interferometry (BLI)

Antigen-binding capacity of the transfected culture supernatants containing individual mAbs was measured by Octet HTX instrument (ForteBio/Sartorius). First, IgG concentration of transiently transfected supernatants containing human mAbs were quantitated with a Protein A biosensor. Then the IgG concentration of the supernatants was adjusted individually to 10 μg/mL in PBS, 1% BSA pH 7.2. Either Protein A or Anti-human Fc coupled to the biosensor were used to capture human mAbs. Following establishment of a baseline signal in the appropriate matrix (PBS, 1% BSA pH 7.2) for 1 min, transfection culture supernatants containing expressed human mAbs diluted to 10 μg/mL were loaded for 5 min onto the biosensors and then washed with the respective matrix medium for 3 min. The F-protein was then associated at 50 nM onto the captured antibodies for 5 min followed by washing with the appropriate matched matrix medium for 3 min. BLI data acquisition and analyses were accomplished with Octet Data Acquisition and Analysis software, version 12. Antigen-binding capacity of the antibodies was reported as a change of wavelength (Response [nm] shift) on the Octet sensorgram. Results of the binding response of PIV1-8 and hMPV-2 to stabilized hPIV1, hPIV3 and hMPV F protein trimers in pre-fusion conformation are shown below in Table 79.

TABLE 79
Binding of hPIV1-8 and hMPV-2 to stabilized hPIV1,
hPIV3 and hMPV F protein trimer in pre-fusion conformation
as determined by BioLayer Interferometry

| | Binding response BLI [nm] | | |
|---|---|---|---|
| mAb ID | HPIV1 | HPIV3 | hMPV |
| hPIV1-8 | 1.96 | −5.2E−02 | −5.1E−02 |
| hMPV-2 | Not | Not | 1.21 |
| | determined | determined | |

### Example 36: Detection of HPIV1-8 and HMPV-2 Antibodies Antigen Binding Ability by Enzyme-Linked Immunosorbant Assay (ELISA)

ELISA was performed to measure the binding ability of the purified monoclonal antibodies (mAb) hPIV1-8 and hMPV-2 for stabilized hPIV1 and hMPV F protein trimer in pre-fusion conformation. Briefly, 96-well microtiter plates were coated with 100 μL/well of individual F protein at 2 μg/mL, and then incubated at 4° C. overnight. After washing with 0.01M TBS/0.1% Brij-35, the plates were blocked with 5% non-fat milk for 1 hr at RT. Then, 100 μL/well of a 1:4 dilution series, starting at 25 nM of the purified mAbs was added to the plates and incubated for 1 hr at RT. After 3 washes with 0.01M TBS/0.1% Brij-35, 100 μL/well of secondary antibody (HRP-anti-human Fc) in a 1:3000 dilution was added to the plates and incubated for 1 hr RT. After 3 washes with 0.01M TBS/0.1% Brij-35, 100 μL/well of TMB-ELISA Substrate Solution was added to the plates. The reaction was stopped with addition of 100 μL/well of 2N H2SO4 after 2 minutes. The plates were then read on a SpectraMax microplate reader with SoftMax Pro software at OD 450 nm (Molecular Devices). The data was analyzed with Graphpad Prism software. EC50 values are shown in Table 80. The data indicate that the hPIV1-8 antibody binds to the prefusion conformation of hPIV1 F protein with high affinity and hMPV-2 binds to the prefusion conformation of hMPV F protein with high affinity.

TABLE 80
Binding of hPVI1-8 and hMPV-2 to stabilized HPIV1 and
hMPV pre-fusion F protein as determined by ELISA

| | HPIV1 pre-fusion F | hMPV pre-fusion F |
|---|---|---|
| | protein binding EC50 | protein binding EC50 |
| mAb ID | [nM] | [nM] |
| hPIV1-8 | 0.103 | Not determined |
| hMPV-2 | Not determined | 0.097 |

### Example 37: Evaluation of Hpiv and hMPV Binding Using Surface Plasmon Resonance (SPR, BIACORE)

Surface Plasmon Resonance, a label-free technology was used to determine affinity of discovered monoclonal antibodies targeting stabilized PIV and/or hMPV F protein trimer in pre fusion or post fusion form as set out in Table 81 below. To avoid conformation changes of F protein while coupling to SPR sensor chip an orientation-specific binding of antibodies through constant (Fc) region was chosen to determine affinity of the newly discovered antibodies using Biacore 8K+ system (Cytiva) and corresponding InSight Software (Cytiva) for analysis. Antibodies had been diluted in 1×HBS-EP+ (10 mM Hepes, 3 mM EDTA, 0.05% Tween20, pH 7.6, Teknova) to 2.5 ug/mL in 339 L total volume (262 μL line1, 77 μl line2) for capture to Protein A Chip (Cytiva) for 60 see with 10 μL/mL flow rate. Unbound antibodies were washed off with 1×HBS-P buffer. Binding affinity was determined by two-fold dilutions series of PIV/hMPV F protein starting from 10 μg/mL to 0.3 μg/mL with seven titration points and a contact time for 60 see at a flow rate of 30 μL/min. Dissociation time was set to 600 seconds to allow for elongated fitting model. Chip was regenerated with two cycles of 4.5M MgCI2 solution. The numbers shown in Table 81 are the equilibrium dissociation constant (KD) calculated as ratio of Koff/Kon between the antibody and the target PIV/hMPV F protein. Protein A and protein G chips had been used to bind first the antibodies via constant region followed by determination of association to F-protein in buffer solution. Affinity of antibodies were reported in molar concentrations as ratio of dissociation and association. As shown in Table 81, hPIV1-8 mAb demonstrated high binding affinity to preF-hPIV1, but no binding to postF-hPIV1. hMPV-2 mAbs demonstrated high affinity binding to preF-hMPV.

TABLE 81
Binding of hPIV1-8 and hMPV-2 to stabilised hPIV1
and hMPV F protein trimer as determined by SPR

Kd (SPR)

| mAb ID | preF-hPIV1 | postF-hPIV1 | preF-hMPV |
|---|---|---|---|
| hPIV1-8 | 8.85E−10 | 0 (no binding) | Not determined |
| hMPV-2 | Not determined | Not determined | 6.85E−10 |

### Example 38: Viral Neutralization Activities of MABS Against hMPV or PIV1 Measured by Neutralization Assay

HPIV1 and hMPV neutralization assays were performed as described with minor modifications (Eyles et al., 2013, J Inf Dis. 208(2):319-29). Briefly, serial dilutions of purified hPIV1 F- or hMPV F-mAbs were mixed with either hPIV1, hMPV A, or hMPV B and transferred to a mammalian cell monolayer. The mixture containing the cells, virus, and purified mAb was incubated for 22-24 hours. After the incubation, fluorescently labeled viral foci were enumerated by a CTL Immunospot Analyzer (Cellular Technology Limited). The data were expressed as IC50 that were calculated as the concentration of the mAb resulting in a 50% reduction in infectious units compared to control wells without the mAb. Results are shown in Table 82.

TABLE 82
Neutralization values for PIV1 F and hMPV F mAbs.

| mAb | Virus | IC50 (μg/ml) |
|---|---|---|
| hPIV1-8 | hPIV1 | 0.005 |
| hMPV-2 | hMPV A | 19.997 |
| hMPV-2 | hMPV B | 23.204 |

### L. Listing of Raw Sequences

SEQ ID NO: 7: Amino acid Sequence of the T4 Fibritin Foldon:
GYIPEAPRDGQAYVRKDGEWVLLSTFL
SEQ ID NO: 8: Linker Sequence:
GS
SEQ ID NO: 9: Linker Sequence:
GGGS
SEQ ID NO: 10: Linker Sequence:
SAIG
SEQ ID NO: 128 - hMPVA precursor polypeptide (ectodomain with foldon and tags)
MSWKWVIIFSLLITPQHGLKESYLEESCSTITEGYLSVLRTGWYTNVFTLEVGDVENLTCAD
GPSLIKTELDLTKSALRELKTVSADQLAREEQIENPRQSRFVLGAIALGVATAAAVTAGVAIA
KTIRLESEVTAIKNALKKTNEAVSTLGNGVRVLATAVRELKDFVSKNLTRAINKNKCDIDDLK
MAVSFSQFNRRFLNVVRQFSDNAGITPAISLDLMTDAELARAVSNMPTSAGQIKLMLENRA
MVRRKGFGILIGVYGSSVIYMVQLPIFGVIDTPCWIVKAAPSCSEKKGNYACLLREDQGWYC
QNAGSTVYYPNEKDCETRGDHVFCDTAAGINVAEQSKECNINISTTNYPCKVSTGRHPISM
VALSPLGALVACYKGVSCSIGSNRVGIIKQLNKGCSYITNQDADTVTIDNTVYQLSKVEGEQH
VIKGRPVSSSFDPVKFPEDQFNVALDQVFENIENSQALVDQSNRILSSAEKGNTGGGSGYIP
EAPRDGQAYVRKDGEWVLLSTFLGRSLEVLFQGPGSAWSHPQFEKAG
Annotated sequence of the hMPV-A precursor polypeptide. Underlined sequence represents
the signal peptide, bolded sequence represents F2 polypeptide, and underlined and bolded
sequence represents the T4 fibritin foldon, PreScission cleavage site, Strep Tag II and linker
sequences.
SEQ ID NO: 129 - hMPV B precursor polypeptide (ectodomain with foldon and tags)
MSWKVMIIISLLITPQHGLKESYLEESCSTITEGYLSVLRTGWYTNVFTLEVGDVENLTCTDG
PSLIKTELDLTKSALRELKTVSADQLAREEQIENPRQSRFVLGAIALGVATAAAVTAGIAIAK
TIRLESEVNAIKGALKTTNEAVSTLGNGVRVLATAVRELKEFVSKNLTSAINKNKCDIADLKMA
VSFSQFNRRFLNVVRQFSDNAGITPAISLDLMNDAELARAVSYMPTSAGQIKLMLENRAMV
RRKGFGILIGVYGSSVIYMVQLPIFGVINTPCWIIKAAPSCSEKDGNYACLLREDQGWYCKNA
GSTVYYPNEKDCETRGDHVFCDTAAGINVAEQSRECNINISTTNYPCKVSTGRHPISMVALS
PLGALVACYKGVSCSIGSNQVGIIKQLPKGCSYITNQDADTVTIDNTVYQLSKVEGEQHVIKG
RPVSSSFDPIRFPEDQFNVALDQVFESIENSQALVDQSNKILNSAEKGNTGGGSGYIPEAPR
DGQAYVRKDGEWVLLSTFLGRSLEVLFQGPGSAWSHPQFEKAG
Annotated sequence of the hMPV-B precursor polypeptide. Underlined sequence represents
the signal peptide, bolded sequence represents F2 polypeptide, and underlined and bolded
sequence represents the T4 fibritin foldon, PreScission cleavage site, Strep Tag II and linker
sequences.
SEQ ID NO: 211 - PIV1 precursor polypeptide (ectodomain with foldon and tags)
MQSSEILLLVYSSLLLSSSLCQIPVDKLSNVGVIINEGKLLKIAGSYESRYIVLSLVPSIDLQDG
CGTTQIIQYKNLLNRLLIPLKDALDLQESLITITNDTTVTNDNPQTRFFGAVIGTIALGVATAA
QITAGIALAEAREARKDIALIKDSIVKTHNSVEFIQRGIGEQIIALKTLQDFVNDEIRPAIGELRC
ETTALKLGIKLTQHYSELATAFSSNLGTIGEKSLTLQALSSLYSANITEILSTIKKDKSDIYDIIYT
EQVKGTVIDVDLEKYMVTLLVKIPILSEIPGVLIYRASSISYNIEGEEWHVAIPNYIINKASSLGG
ADVTNCIESKLAYICPRDPTQLIPDNQQKCILGDVSKCPVTKVINNLVPKFAFINGGVVANCIA
STCTCGTNRIPVNQDRSKGVTFLTYTNCGLIGINGIELYANKRGRDTTWGNQIIKVGPAVSIR
PVDISLNLASATNFLEESKTELGGGSGYIPEAPRDGQAYVRKDGEWVLLSTFLGRSLEVLF
QGPGSAWSHPQFEKAG
Annotated sequence of the PIV1 precursor polypeptide. Underlined sequence represents the
signal peptide, bolded sequence represents F2 polypeptide, and underlined and bolded
sequence represents the T4 fibritin foldon, PreScission cleavage site, Strep Tag II and linker
sequences.
SEQ ID NO: 305 - PIV3 precursor polypeptide (ectodomain with foldon and tags)
MLISILLIITTMIMASHCQIDITKLQHVGVLVNSPKGMKISQNFETRYLILSLIPKIEDSNSCGDQ
QIKQYKRLLDRLIIPLYDGLRLQKDVIVTNQESNENTDPRTERFFGGVIGTIALGVATSAQITA
AVALVEAKQARSDIEKLKEAIRDTNKAVQSVQSSVGNLIVAIKSVQDYVNKEIVPSIARLGCEA
AGLQLGIALTQHYSELTNIFGDNIGSLQEKGIKLQGIASLYRTNITEIFTTSTVDKYDIYDLLFTE
SIKVRVIDVDLNDYSITLQVRLPLLTRLLNTQIYKVDSISYNIQNREWYIPLPSHIMTKGAFLGG
ADVKECIEAFSSYICPSDPGFVLNHEMESCLSGNISQCPRTTVTSDIVPRYAFVNGGVVANCI
TTTCTCNGIGNRINQPPDQGVKIITHKECNTIGINGMLFNTNKEGTLAFYTPDDITLNNSVALD
PIDISIELNKAKSDLEESKEWIRRSNQKLGGGSGYIPEAPRDGQAYVRKDGEWVLLSTFLGR
SLEVLFQGPGSAWSHPQFEKAG
Annotated sequence of the PIV3 precursor polypeptide. Underlined sequence represents the
signal peptide, bolded sequence represents F2 polypeptide, and underlined and bolded
sequence represents the T4 fibritin foldon, PreScission cleavage site, Strep tag II and linker
sequences.
SEQ ID NO: 358: Amino Acid Sequence of Heavy Chain Variable Domain of Antibody
MPE8 mAb:
EVQLVESGGGLVKPGGSLRLSCAASGFTFSSYSMNWVRQAPGKGLEWVSSISASSSYSDY
ADSAKGRFTISRDNAKTSLFLQMNSLRAEDTAIYFCARARATGYSSITPYFDIWGQGTLVTV
SS
SEQ ID NO: 359: Amino Acid Sequence of Light Chain Variable Domain of Antibody
MPE8 mAb:
QSVVTQTPSVSGAPGQRVTISCTGSSSNIGAGYDVHWYQQLPGTAPKLLIYDNNNRPSGVP
DRFSASKSGTSASLAITGLQAEDEADYYCQSYDRNLSGVFGTGTKVTVL
SEQ ID NO: 360: Amino Acid Sequence of Heavy Chain Variable Domain of Antibody
hMPV-2 mAb:
QVQLQQSGAEVKKPGASVKVSCKASGYTFTSYGISWVRQAPGQGLEWMGWISGYNGNTN
YAQTFQGTFTMTTDTSTSTAYMELRRLRSGDTAVYYCARDRYYASGSYNGMDVWGQGTT
VTVSS
SEQ ID NO: 361: Amino Acid Sequence of Light Chain Variable Domain of Antibody
hMPV-2 mAb:
QSALTQPPSASGAPGQRVTVSCSGSSSNVGSSSVYWYQQLPGTAPKLLIYRNNQRPSGVP
DRFSGSKSGTSASLAISGLRSEDEADYYCAAWDDSLSGWVFGGGTKLTVL
SEQ ID NO: 362: Amino Acid Sequence of Heavy Chain Variable Domain of Antibody
PIV1-8 mAb:
QVQLVQSGAEVKKPGSSVKVSCEASGGTFSNYVISWVRQAPGQGLEWMGGIIPVFATAKY
AQKFQGRVTIIADQSTNTAYLELSSLRSDDTAVYFCASVDSGGTWWDAFAIWGQGTMVTVS
S
SEQ ID NO: 363: Amino Acid Sequence of Light Chain Variable Domain of Antibody
PIV1-8 mAb:
QAVVTQPRSVSGSPGQAVTISCTGTSSDVGGYNYVSWYQHHPGKAPKLIIYDVTKRPSGVP
DRFSGSKSGNTASLTISGLQTEYEADYYCCSYAGSYTLVFGGGTKLTVL
SEQ ID NO: 364: Amino Acid Sequence of Heavy Chain Variable Domain of Antibody
PIA174 mAb:
VQLQESGPGLVKPSETLSLTCTVSGGSVSSYYWSWIRQPPGKGLEWIGNIYYSGTTKYNPS
LKSRVTISVDVSKNQFSLNLISVTAADTAVYFCARQVKSGWFVQPFDYWGQGALVTVSSAS
TKGPSVFPLAPSSKSTSGGTAALGCLVKDYFPEPVTVSWNSGALTSGVHTFPAVLQSSGLY
SLSSVVTVPSSSLGTQTYICNVNHKPSNTKVDKKVEPK
SEQ ID NO: 365: Amino Acid Sequence of Light Chain Variable Domain of Antibody
PIA174 mAb:
QMTQSPPSLSAYVGDRVTITCRASQAIANYLAWFQQKPGKAPKSLIYAASTLQSGVPSRFS
GSGSGTDFTLTISSLQPEDFATYYCHQYNTYPITFGQGTRLEIKRRTVAAPSVFIFPPSDEQL
KSGTASVVCLLNNFYPREAKVQWKVDNALQSGNSQESVTEQDSKDSTYSLSSTLTLSKAD
YEKHKVYACEVTHQGLSSPVTKS
SEQ ID NO: 553: Amino Acid Sequence of Heavy Chain Variable Domain of Antibody
PIA174 mAb:
VQLQESGPGLVKPSETLSLTCTVSGGSVSSYYWSWIRQPPGKGLEWIGNIYYSGTTKYNPS
LKSRVTISVDVSKNQFSLNLISVTAADTAVYFCARQVKSGWFVQPFDYWGQGALVTVSS
SEQ ID NO: 554: Amino Acid Sequence of Light Chain Variable Domain of Antibody
PIA174 mAb:
QMTQSPPSLSAYVGDRVTITCRASQAIANYLAWFQQKPGKAPKSLIYAASTLQSGVPSRFS
GSGSGTDFTLTISSLQPEDFATYYCHQYNTYPITFGQGTRLEIK
>hMPV046_DNA (SEQ ID NO: 390)
ATGAGCTGGAAGGTGGTCATCATCTTCAGCCTGCTGATCACCCCTCAGCACGGCCTGA
AAGAGAGCTACCTGGAAGAAAGCTGCAGCACCATCACCGAGGGCTACCTGAGCGTGCT
GAGAACCGGCTGGTACACCAACGTGTTCACCCTGGAAGTGGGCGACGTGGAAAACCTG
ACCTGTGCCGATGGACCCAGCCTGATCAAGACCGAGCTGGACCTGACAAAGAGCGCC
CTGCGGGAACTGAAAACCGTGTCTGCAGATCAGCTGGCCAGAGAGGAACAGATCGAGA
ACCCCAGACAGAGCAGATTCGTGCTGGGAGCTATCGCCCTGGGAGTTGCTACAGCTGC
TGCTGTGACAGCTGGCGTGGCCATTGCCAAGACCATCCGGCTGGAAAGCGAAGTGACC
GCCATCAAGAACTGCCTGAAAAAGACCAACGAGGCCGTGTGCACCCTCGGCAATGGCG
TTAGAGTGCTGGCCACAGCCGTCCGCGAGCTGAAGGATTTCGTGTCCAAGAACCTGAC
CAGGGCCATCAACAAGAACAAGTGCGACATCGACGACCTGAAGATGGCCGTGTCCTTC
AGCCAGTTCAACCGGCGGTTCCTGAATGTCGTGCGGCAGTTCTCTGACAACGCCGGCA
TCACACCAGCCATCAGCCTGGATCTGATGACCGATGCCGAACTGGCTAGAGCCGTGTC
CAACATGCCTACATCTGCCGGCCAGATCAAGCTGATGCTGGAAAACAGAGCCATGGTC
CGACGGAAAGGCTTCGGCATCCTGATCGGCGTGTACGGCAGCAGCGTGATCTACATGG
TGCAGCTGCCTATCTTCGGCGTGATCGACACCCCTTGCTGGATCGTGAAAGCCGCTCC
TAGCTGCAGCGAGAAGAAGGGCAATTACGCCTGCCTGCTGAGAGAGGACCAAGGCTG
GTACTGTCAGAATGCCGGCAGCACCGTGTACTACCCCAACGAGAAGGACTGCGAGACA
AGAGGCGACCACGTGTTCTGTGATACCGCCGCTGGAATCAACGTGGCCGAGCAGAGC
AAAGAGTGCAACATCAACATCAGCACCACAAACTACCCCTGCAAGGTGTCCACCGGCA
GACACCCTATCAGCATGGTGGCTCTGTCTCCACTGGGAGCCCTGGTGGCTTGTTATAA
GGGCGTGTCCTGTAGCATCGGCAGCAACAGAGTGGGCATCATCAAGCAGCTGAACAAG
GGCTGCTCCTACATCACCAACCAGGACGCCGATACCGTGACCATCGACAATACCGTGT
ATCAGCTGAGCAAGGTGGAAGGCGAACAGCACGTGATCAAGGGCAGACCTGTGTCCA
GCAGCTTCGACCCCGTGAAGTTCCCCGAGGACCAGTTCAATGTGGCCCTGGACCAGGT
GTTCGAGAACATCGAGAACTCTCAGGCTCTGGTGGACCAGTCCAACCGGATTCTGTCTA
GCGCCGAGAAGGGAAACACCGGCTTCATCATCGTGATCATCCTGATTGCCGTGCTGGG
CTCCAGCATGATCCTGGTGTCCATCTTTATCATCATCAAAAAGACGAAGAAGCCCACAG
GCGCCCCTCCAGAACTGTCTGGCGTGACCAACAATGGCTTCATCCCTCACAGC
>hMPV046_mRNA (SEQ ID NO: 391)
AUGAGCUGGAAGGUGGUCAUCAUCUUCAGCCUGCUGAUCACCCCUCAGCACGGCCU
GAAAGAGAGCUACCUGGAAGAAAGCUGCAGCACCAUCACCGAGGGCUACCUGAGCGU
GCUGAGAACCGGCUGGUACACCAACGUGUUCACCCUGGAAGUGGGCGACGUGGAAA
ACCUGACCUGUGCCGAUGGACCCAGCCUGAUCAAGACCGAGCUGGACCUGACAAAGA
GCGCCCUGCGGGAACUGAAAACCGUGUCUGCAGAUCAGCUGGCCAGAGAGGAACAG
AUCGAGAACCCCAGACAGAGCAGAUUCGUGCUGGGAGCUAUCGCCCUGGGAGUUGC
UACAGCUGCUGCUGUGACAGCUGGCGUGGCCAUUGCCAAGACCAUCCGGCUGGAAA
GCGAAGUGACCGCCAUCAAGAACUGCCUGAAAAAGACCAACGAGGCCGUGUGCACCC
UCGGCAAUGGCGUUAGAGUGCUGGCCACAGCCGUCCGCGAGCUGAAGGAUUUCGUG
UCCAAGAACCUGACCAGGGCCAUCAACAAGAACAAGUGCGACAUCGACGACCUGAAG
AUGGCCGUGUCCUUCAGCCAGUUCAACCGGCGGUUCCUGAAUGUCGUGCGGCAGUU
CUCUGACAACGCCGGCAUCACACCAGCCAUCAGCCUGGAUCUGAUGACCGAUGCCGA
ACUGGCUAGAGCCGUGUCCAACAUGCCUACAUCUGCCGGCCAGAUCAAGCUGAUGC
UGGAAAACAGAGCCAUGGUCCGACGGAAAGGCUUCGGCAUCCUGAUCGGCGUGUAC
GGCAGCAGCGUGAUCUACAUGGUGCAGCUGCCUAUCUUCGGCGUGAUCGACACCCC
UUGCUGGAUCGUGAAAGCCGCUCCUAGCUGCAGCGAGAAGAAGGGCAAUUACGCCU
GCCUGCUGAGAGAGGACCAAGGCUGGUACUGUCAGAAUGCCGGCAGCACCGUGUAC
UACCCCAACGAGAAGGACUGCGAGACAAGAGGCGACCACGUGUUCUGUGAUACCGC
CGCUGGAAUCAACGUGGCCGAGCAGAGCAAAGAGUGCAACAUCAACAUCAGCACCAC
AAACUACCCCUGCAAGGUGUCCACCGGCAGACACCCUAUCAGCAUGGUGGCUCUGU
CUCCACUGGGAGCCCUGGUGGCUUGUUAUAAGGGCGUGUCCUGUAGCAUCGGCAGC
AACAGAGUGGGCAUCAUCAAGCAGCUGAACAAGGGCUGCUCCUACAUCACCAACCAG
GACGCCGAUACCGUGACCAUCGACAAUACCGUGUAUCAGCUGAGCAAGGUGGAAGG
CGAACAGCACGUGAUCAAGGGCAGACCUGUGUCCAGCAGCUUCGACCCCGUGAAGU
UCCCCGAGGACCAGUUCAAUGUGGCCCUGGACCAGGUGUUCGAGAACAUCGAGAAC
UCUCAGGCUCUGGUGGACCAGUCCAACCGGAUUCUGUCUAGCGCCGAGAAGGGAAA
CACCGGCUUCAUCAUCGUGAUCAUCCUGAUUGCCGUGCUGGGCUCCAGCAUGAUCC
UGGUGUCCAUCUUUAUCAUCAUCAAAAAGACGAAGAAGCCCACAGGCGCCCCUCCAG
AACUGUCUGGCGUGACCAACAAUGGCUUCAUCCCUCACAGC
>hMPV078_DNA (SEQ ID NO: 392)
ATGAGCTGGAAGGTGGTCATCATCTTCAGCCTGCTGATCACCCCTCAGCACGGCCTGA
AAGAGAGCTACCTGGAAGAAAGCTGCAGCACCATCACCGAGGGCTACCTGAGCGTGCT
GAGAACCGGCTGGTACACCAACGTGTTCACCCTGGAAGTGGGCGACGTGGAAAACCTG
ACCTGTGCCGATGGACCCAGCCTGATCAAGACCGAGCTGGACCTGACAAAGAGCGCC
CTGCGGGAACTGAAAACCGTGTCTGCAGATCAGCTGGCCAGAGAGGAACAGATCGAGA
ACCCCAGACAGAGCAGATTCGTGCTGGGAGCTATCGCCCTGGGAGTTGCTACAGCTGC
TGCTGTGACAGCTGGCGTGGCCATTGCCAAGACCATCCGGCTGGAAAGCGAAGTGACC
GCCATCAAGAACTGCCTGAAAAAGACCAACGAGGCCGTGTGCACCCTCGGCAATGGCG
TTAGAGTGCTGGCCACAGCCGTCCGCGAGCTGAAGGATTTCGTGTCCAAGAACCTGAC
CAGGGCCATCAACAAGAACAAGTGCGACATCGACGACCTGAAGATGGCCGTGTCCTTC
AGCCAGTTCAACCGGCGGTTCCTGAATGTCGTGCGGCAGTTCTCTGACAACGCCGGCA
TCACACCAGCCATCAGCCTGGATCTGATGACCGATGCCGAACTGGCTAGAGCCGTGTC
CAACATGCCTACATCTGCCGGCCAGATCAAGCTGATGCTGGAAAACAGAGCCATGGTC
CGACGGAAAGGCTTCGGCATCCTGATCGGCGTGTACGGCAGCAGCGTGATCTACATGG
TGCAGCTGCCTATCTTCGGCGTGATCGACACCCCTTGCTGGATCGTGAAAGCCGCTCC
TAGCTGCAGCGAGAAGAAGGGCAATTACGCCTGCCTGCTGAGAGAGGACCAAGGCTG
GTACTGTCAGAATGCCGGCAGCACCGTGTACTACCCCAACGAGAAGGACTGCGAGACA
AGAGGCGACCACGTGTTCTGTGATACCGCCGCTGGAATCAACGTGGCCGAGCAGAGC
AAAGAGTGCAACATCAACATCAGCACCACAAACTACCCCTGCAAGGTGTCCACCGGCA
GACACCCTATCAGCATGGTGGCTCTGTCTCCACTGGGAGCCCTGGTGGCTTGTTATAA
GGGCGTGTCCTGTAGCATCGGCAGCAACAGAGTGGGCATCATCAAGCAGCTGAACAAG
GGCTGCTCCTACATCTGCAACCAGGACGCCGATACCGTGACCATCGACAATACCGTGT
ATCAGCTGAGCAAGGTGGAAGGCGAATGCCACGTGATCAAGGGCAGACCTGTGTCCAG
CAGCTTCGACCCCGTGAAGTTCCCCGAGGACCAGTTCAATGTGGCCCTGGACCAGGTG
TTCGAGAACATCGAGAACTCTCAGGCTCTGGTGGACCAGTCCAACCGGATTCTGTCTAG
CGCCGAGAAGGGAAACACCGGCTTCATCATCGTGATCATCCTGATTGCCGTGCTGGGC
TCCAGCATGATCCTGGTGTCCATCTTTATCATCATCAAAAAGACGAAGAAGCCCACAGG
CGCCCCTCCAGAACTGTCTGGCGTGACCAACAATGGCTTCATCCCTCACAGC
>hMPV078_mRNA (SEQ ID NO: 393)
AUGAGCUGGAAGGUGGUCAUCAUCUUCAGCCUGCUGAUCACCCCUCAGCACGGCCU
GAAAGAGAGCUACCUGGAAGAAAGCUGCAGCACCAUCACCGAGGGCUACCUGAGCGU
GCUGAGAACCGGCUGGUACACCAACGUGUUCACCCUGGAAGUGGGCGACGUGGAAA
ACCUGACCUGUGCCGAUGGACCCAGCCUGAUCAAGACCGAGCUGGACCUGACAAAGA
GCGCCCUGCGGGAACUGAAAACCGUGUCUGCAGAUCAGCUGGCCAGAGAGGAACAG
AUCGAGAACCCCAGACAGAGCAGAUUCGUGCUGGGAGCUAUCGCCCUGGGAGUUGC
UACAGCUGCUGCUGUGACAGCUGGCGUGGCCAUUGCCAAGACCAUCCGGCUGGAAA
GCGAAGUGACCGCCAUCAAGAACUGCCUGAAAAAGACCAACGAGGCCGUGUGCACCC
UCGGCAAUGGCGUUAGAGUGCUGGCCACAGCCGUCCGCGAGCUGAAGGAUUUCGUG
UCCAAGAACCUGACCAGGGCCAUCAACAAGAACAAGUGCGACAUCGACGACCUGAAG
AUGGCCGUGUCCUUCAGCCAGUUCAACCGGCGGUUCCUGAAUGUCGUGCGGCAGUU
CUCUGACAACGCCGGCAUCACACCAGCCAUCAGCCUGGAUCUGAUGACCGAUGCCGA
ACUGGCUAGAGCCGUGUCCAACAUGCCUACAUCUGCCGGCCAGAUCAAGCUGAUGC
UGGAAAACAGAGCCAUGGUCCGACGGAAAGGCUUCGGCAUCCUGAUCGGCGUGUAC
GGCAGCAGCGUGAUCUACAUGGUGCAGCUGCCUAUCUUCGGCGUGAUCGACACCCC
UUGCUGGAUCGUGAAAGCCGCUCCUAGCUGCAGCGAGAAGAAGGGCAAUUACGCCU
GCCUGCUGAGAGAGGACCAAGGCUGGUACUGUCAGAAUGCCGGCAGCACCGUGUAC
UACCCCAACGAGAAGGACUGCGAGACAAGAGGCGACCACGUGUUCUGUGAUACCGC
CGCUGGAAUCAACGUGGCCGAGCAGAGCAAAGAGUGCAACAUCAACAUCAGCACCAC
AAACUACCCCUGCAAGGUGUCCACCGGCAGACACCCUAUCAGCAUGGUGGCUCUGU
CUCCACUGGGAGCCCUGGUGGCUUGUUAUAAGGGCGUGUCCUGUAGCAUCGGCAGC
AACAGAGUGGGCAUCAUCAAGCAGCUGAACAAGGGCUGCUCCUACAUCUGCAACCAG
GACGCCGAUACCGUGACCAUCGACAAUACCGUGUAUCAGCUGAGCAAGGUGGAAGG
CGAAUGCCACGUGAUCAAGGGCAGACCUGUGUCCAGCAGCUUCGACCCCGUGAAGU
UCCCCGAGGACCAGUUCAAUGUGGCCCUGGACCAGGUGUUCGAGAACAUCGAGAAC
UCUCAGGCUCUGGUGGACCAGUCCAACCGGAUUCUGUCUAGCGCCGAGAAGGGAAA
CACCGGCUUCAUCAUCGUGAUCAUCCUGAUUGCCGUGCUGGGCUCCAGCAUGAUCC
UGGUGUCCAUCUUUAUCAUCAUCAAAAAGACGAAGAAGCCCACAGGCGCCCCUCCAG
AACUGUCUGGCGUGACCAACAAUGGCUUCAUCCCUCACAGC
>hMPV079_DNA (SEQ ID NO: 394)
ATGAGCTGGAAGGTGGTCATCATCTTCAGCCTGCTGATCACCCCTCAGCACGGCCTGA
AAGAGAGCTACCTGGAAGAAAGCTGCAGCACCATCACCGAGGGCTACCTGAGCGTGCT
GAGAACCGGCTGGTACACCAACGTGTTCACCCTGGAAGTGGGCGACGTGGAAAACCTG
ACCTGTGCCGATGGACCCAGCCTGATCAAGACCGAGCTGGACCTGACAAAGAGCGCC
CTGCGGGAACTGAAAACCGTGTCTGCAGATCAGCTGGCCAGAGAGGAACAGATCGAGA
ACCCCAGACAGAGCAGATTCGTGCTGGGAGCTATCGCCCTGGGAGTTGCTACAGCTGC
TGCTGTGACAGCTGGCGTGGCCATTGCCAAGACCATCCGGCTGGAAAGCGAAGTGACC
GCCATCAAGAACTGCCTGAAAAAGACCAACGAGGCCGTGTGCACCCTCGGCAATGGCG
TTAGAGTGCTGGCCACAGCCGTCCGCGAGCTGAAGGATTTCGTGTCCAAGAACCTGAC
CAGGGCCATCAACAAGAACAAGTGCGACATCGACGACCTGAAGATGGCCGTGTCCTTC
AGCCAGTTCAACCGGCGGTTCCTGAATGTCGTGCGGCAGTTCTCTGACAACGCCGGCA
TCACACCAGCCATCAGCCTGGATCTGATGACCGATGCCGAACTGGCTAGAGCCGTGTC
CAACATGCCTACATCTGCCGGCCAGATCAAGCTGATGCTGGAAAACAGAGCCATGGTC
CGACGGAAAGGCTTCGGCATCCTGATCGGCGTGTACGGCAGCAGCGTGATCTACATGG
TGCAGCTGCCTATCTTCGGCGTGATCGACACCCCTTGCTGGATCGTGAAAGCCGCTCC
TAGCTGCAGCGAGAAGAAGGGCAATTACGCCTGCCTGCTGAGAGAGGACCAAGGCTG
GTACTGTCAGAATGCCGGCAGCACCGTGTACTACCCCAACGAGAAGGACTGCGAGACA
AGAGGCGACCACGTGTTCTGTGATACCGCCGCTGGAATCAACGTGGCCGAGCAGAGC
AAAGAGTGCAACATCAACATCAGCACCACAAACTACCCCTGCAAGGTGTCCACCGGCA
GACACCCTATCAGCATGGTGGCTCTGTCTCCACTGGGAGCCCTGGTGGCTTGTTATAA
GGGCGTGTCCTGTAGCATCGGCAGCAACAGAGTGGGCATCATCAAGCAGCTGAACAAG
GGCTGCTCCTACATCTGCAACCAGGACGCCGATACCGTGACCATCGACAATACCGTGT
ATCAGCTGAGCAAGGTGGAAGGCGAATGCCACGTGATCAAGGGCAGACCTGTGTCCAG
CAGCTTCGACCCCGTGAAGTTCCCCGAGGACCAGTTCAATGTGCCCCTGGACCAGGTG
TTCGAGAACATCGAGAACTCTCAGGCTCTGGTGGACCAGTCCAACCGGATTCTGTCTAG
CGCCGAGAAGGGAAACACCGGCTTCATCATCGTGATCATCCTGATTGCCGTGCTGGGC
TCCAGCATGATCCTGGTGTCCATCTTTATCATCATCAAAAAGACGAAGAAGCCCACAGG
CGCCCCTCCAGAACTGTCTGGCGTGACCAACAATGGCTTCATCCCTCACAGC
>hMPV079_mRNA (SEQ ID NO: 395)
AUGAGCUGGAAGGUGGUCAUCAUCUUCAGCCUGCUGAUCACCCCUCAGCACGGCCU
GAAAGAGAGCUACCUGGAAGAAAGCUGCAGCACCAUCACCGAGGGCUACCUGAGCGU
GCUGAGAACCGGCUGGUACACCAACGUGUUCACCCUGGAAGUGGGCGACGUGGAAA
ACCUGACCUGUGCCGAUGGACCCAGCCUGAUCAAGACCGAGCUGGACCUGACAAAGA
GCGCCCUGCGGGAACUGAAAACCGUGUCUGCAGAUCAGCUGGCCAGAGAGGAACAG
AUCGAGAACCCCAGACAGAGCAGAUUCGUGCUGGGAGCUAUCGCCCUGGGAGUUGC
UACAGCUGCUGCUGUGACAGCUGGCGUGGCCAUUGCCAAGACCAUCCGGCUGGAAA
GCGAAGUGACCGCCAUCAAGAACUGCCUGAAAAAGACCAACGAGGCCGUGUGCACCC
UCGGCAAUGGCGUUAGAGUGCUGGCCACAGCCGUCCGCGAGCUGAAGGAUUUCGUG
UCCAAGAACCUGACCAGGGCCAUCAACAAGAACAAGUGCGACAUCGACGACCUGAAG
AUGGCCGUGUCCUUCAGCCAGUUCAACCGGCGGUUCCUGAAUGUCGUGCGGCAGUU
CUCUGACAACGCCGGCAUCACACCAGCCAUCAGCCUGGAUCUGAUGACCGAUGCCGA
ACUGGCUAGAGCCGUGUCCAACAUGCCUACAUCUGCCGGCCAGAUCAAGCUGAUGC
UGGAAAACAGAGCCAUGGUCCGACGGAAAGGCUUCGGCAUCCUGAUCGGCGUGUAC
GGCAGCAGCGUGAUCUACAUGGUGCAGCUGCCUAUCUUCGGCGUGAUCGACACCCC
UUGCUGGAUCGUGAAAGCCGCUCCUAGCUGCAGCGAGAAGAAGGGCAAUUACGCCU
GCCUGCUGAGAGAGGACCAAGGCUGGUACUGUCAGAAUGCCGGCAGCACCGUGUAC
UACCCCAACGAGAAGGACUGCGAGACAAGAGGCGACCACGUGUUCUGUGAUACCGC
CGCUGGAAUCAACGUGGCCGAGCAGAGCAAAGAGUGCAACAUCAACAUCAGCACCAC
AAACUACCCCUGCAAGGUGUCCACCGGCAGACACCCUAUCAGCAUGGUGGCUCUGU
CUCCACUGGGAGCCCUGGUGGCUUGUUAUAAGGGCGUGUCCUGUAGCAUCGGCAGC
AACAGAGUGGGCAUCAUCAAGCAGCUGAACAAGGGCUGCUCCUACAUCUGCAACCAG
GACGCCGAUACCGUGACCAUCGACAAUACCGUGUAUCAGCUGAGCAAGGUGGAAGG
CGAAUGCCACGUGAUCAAGGGCAGACCUGUGUCCAGCAGCUUCGACCCCGUGAAGU
UCCCCGAGGACCAGUUCAAUGUGCCCCUGGACCAGGUGUUCGAGAACAUCGAGAAC
UCUCAGGCUCUGGUGGACCAGUCCAACCGGAUUCUGUCUAGCGCCGAGAAGGGAAA
CACCGGCUUCAUCAUCGUGAUCAUCCUGAUUGCCGUGCUGGGCUCCAGCAUGAUCC
UGGUGUCCAUCUUUAUCAUCAUCAAAAAGACGAAGAAGCCCACAGGCGCCCCUCCAG
AACUGUCUGGCGUGACCAACAAUGGCUUCAUCCCUCACAGC
>hMPV082_DNA (SEQ ID NO: 396)
ATGAGCTGGAAGGTGGTCATCATCTTCAGCCTGCTGATCACCCCTCAGCACGGCCTGA
AAGAGAGCTACCTGGAAGAAAGCTGCAGCACCATCACCGAGGGCTACCTGAGCGTGCT
GAGAACCGGCTGGTACACCAACGTGTTCACCCTGGAAGTGGGCGACGTGGAAAACCTG
ACCTGTGCCGATGGACCCAGCCTGATCAAGACCGAGCTGGACCTGACAAAGAGCGCC
CTGCGGGAACTGAAAACCGTGTCTGCAGATCAGCTGGCCAGAGAGGAACAGATCGAGA
ACCCCAGACAGAGCAGATTCGTGCTGGGAGCTATCGCCCTGGGAGTTGCTACAGCTGC
TGCTGTGACAGCTGGCGTGGCCATTGCCAAGACCATCCGGCTGGAAAGCGAAGTGACC
GCCATCAAGAACTGCCTGAAAAAGACCAACGAGGCCGTGTGCACCCTCGGCAATGGCG
TTAGAGTGCTGGCCACAGCCGTCCGCGAGCTGAAGGATTTCGTGTCCAAGAACCTGAC
CAGGGCCATCAACAAGAACAAGTGCGACATCGACGACCTGAAGATGGCCGTGTCCTTC
AGCCAGTTCAACCGGCGGTTCCTGAATGTCGTGCGGCAGTTCTCTGACAACGCCGGCA
TCACACCAGCCATCAGCCTGGATCTGATGACCGATGCCGAACTGGCTAGAGCCGTGTC
CAACATGCCTACATCTGCCGCCCAGATCAAGCTGATGCTGGAAAACAGAGCCATGGTC
CGACGGAAAGGCTTCGGCATCCTGATCGGCGTGTACGGCAGCAGCGTGATCTACATGG
TGCAGCTGCCTATCTTCGGCGTGATCGACACCCCTTGCTGGATCGTGAAAGCCGCTCC
TAGCTGCAGCGAGAAGAAGGGCAATTACGCCTGCCTGCTGAGAGAGGACCAAGGCTG
GTACTGTCAGAATGCCGGCAGCACCGTGTACTACCCCAACGAGAAGGACTGCGAGACA
AGAGGCGACCACGTGTTCTGTGATACCGCCGCTGGAATCAACGTGGCCGAGCAGAGC
AAAGAGTGCAACATCAACATCAGCACCACAAACTACCCCTGCAAGGTGTCCACCGGCA
GACACCCTATCAGCATGGTGGCTCTGTCTCCACTGGGAGCCCTGGTGGCTTGTTATAA
GGGCGTGTCCTGTAGCATCGGCAGCAACAGAGTGGGCATCATCAAGCAGCTGAACAAG
GGCTGCTCCTACATCTGCAACCAGGACGCCGATACCGTGACCATCGACAATACCGTGT
ATCAGCTGAGCAAGGTGGAAGGCGAATGCCACGTGATCAAGGGCAGACCTGTGTCCAG
CAGCTTCGACCCCGTGAAGTTCCCCGAGGACCAGTTCAATGTGGCCCTGGACCAGGTG
TTCGAGAACATCGAGAACTCTCAGGCTCTGGTGGACCAGTCCAACCGGATTCTGTCTAG
CGCCGAGAAGGGAAACACCGGCTTCATCATCGTGATCATCCTGATTGCCGTGCTGGGC
TCCAGCATGATCCTGGTGTCCATCTTTATCATCATCAAAAAGACGAAGAAGCCCACAGG
CGCCCCTCCAGAACTGTCTGGCGTGACCAACAATGGCTTCATCCCTCACAGC
>hMPV082_mRNA (SEQ ID NO: 397)
AUGAGCUGGAAGGUGGUCAUCAUCUUCAGCCUGCUGAUCACCCCUCAGCACGGCCU
GAAAGAGAGCUACCUGGAAGAAAGCUGCAGCACCAUCACCGAGGGCUACCUGAGCGU
GCUGAGAACCGGCUGGUACACCAACGUGUUCACCCUGGAAGUGGGCGACGUGGAAA
ACCUGACCUGUGCCGAUGGACCCAGCCUGAUCAAGACCGAGCUGGACCUGACAAAGA
GCGCCCUGCGGGAACUGAAAACCGUGUCUGCAGAUCAGCUGGCCAGAGAGGAACAG
AUCGAGAACCCCAGACAGAGCAGAUUCGUGCUGGGAGCUAUCGCCCUGGGAGUUGC
UACAGCUGCUGCUGUGACAGCUGGCGUGGCCAUUGCCAAGACCAUCCGGCUGGAAA
GCGAAGUGACCGCCAUCAAGAACUGCCUGAAAAAGACCAACGAGGCCGUGUGCACCC
UCGGCAAUGGCGUUAGAGUGCUGGCCACAGCCGUCCGCGAGCUGAAGGAUUUCGUG
UCCAAGAACCUGACCAGGGCCAUCAACAAGAACAAGUGCGACAUCGACGACCUGAAG
AUGGCCGUGUCCUUCAGCCAGUUCAACCGGCGGUUCCUGAAUGUCGUGCGGCAGUU
CUCUGACAACGCCGGCAUCACACCAGCCAUCAGCCUGGAUCUGAUGACCGAUGCCGA
ACUGGCUAGAGCCGUGUCCAACAUGCCUACAUCUGCCGCCCAGAUCAAGCUGAUGC
UGGAAAACAGAGCCAUGGUCCGACGGAAAGGCUUCGGCAUCCUGAUCGGCGUGUAC
GGCAGCAGCGUGAUCUACAUGGUGCAGCUGCCUAUCUUCGGCGUGAUCGACACCCC
UUGCUGGAUCGUGAAAGCCGCUCCUAGCUGCAGCGAGAAGAAGGGCAAUUACGCCU
GCCUGCUGAGAGAGGACCAAGGCUGGUACUGUCAGAAUGCCGGCAGCACCGUGUAC
UACCCCAACGAGAAGGACUGCGAGACAAGAGGCGACCACGUGUUCUGUGAUACCGC
CGCUGGAAUCAACGUGGCCGAGCAGAGCAAAGAGUGCAACAUCAACAUCAGCACCAC
AAACUACCCCUGCAAGGUGUCCACCGGCAGACACCCUAUCAGCAUGGUGGCUCUGU
CUCCACUGGGAGCCCUGGUGGCUUGUUAUAAGGGCGUGUCCUGUAGCAUCGGCAGC
AACAGAGUGGGCAUCAUCAAGCAGCUGAACAAGGGCUGCUCCUACAUCUGCAACCAG
GACGCCGAUACCGUGACCAUCGACAAUACCGUGUAUCAGCUGAGCAAGGUGGAAGG
CGAAUGCCACGUGAUCAAGGGCAGACCUGUGUCCAGCAGCUUCGACCCCGUGAAGU
UCCCCGAGGACCAGUUCAAUGUGGCCCUGGACCAGGUGUUCGAGAACAUCGAGAAC
UCUCAGGCUCUGGUGGACCAGUCCAACCGGAUUCUGUCUAGCGCCGAGAAGGGAAA
CACCGGCUUCAUCAUCGUGAUCAUCCUGAUUGCCGUGCUGGGCUCCAGCAUGAUCC
UGGUGUCCAUCUUUAUCAUCAUCAAAAAGACGAAGAAGCCCACAGGCGCCCCUCCAG
AACUGUCUGGCGUGACCAACAAUGGCUUCAUCCCUCACAGC
>hMPV083_DNA (SEQ ID NO: 398)
ATGAGCTGGAAGGTGGTCATCATCTTCAGCCTGCTGATCACCCCTCAGCACGGCCTGA
AAGAGAGCTACCTGGAAGAAAGCTGCAGCACCATCACCGAGGGCTACCTGAGCGTGCT
GAGAACCGGCTGGTACACCAACGTGTTCATCCTGGAAGTGGGCGACGTGGAAAACCTG
ACCTGTGCCGATGGACCCAGCCTGATCAAGACCGAGCTGGACCTGACAAAGAGCGCC
CTGCGGGAACTGAAAACCGTGTCTGCAGATCAGCTGGCCAGAGAGGAACAGATCGAGA
ACCCCAGACAGAGCAGATTCGTGCTGGGAGCTATCGCCCTGGGAGTTGCTACAGCTGC
TGCTGTGACAGCTGGCGTGGCCATTGCCAAGACCATCCGGCTGGAAAGCGAAGTGACC
GCCATCAAGAACTGCCTGAAAAAGACCAACGAGGCCGTGTGCACCCTCGGCAATGGCG
TTAGAGTGCTGGCCACAGCCGTCCGCGAGCTGAAGGATTTCGTGTCCAAGAACCTGAC
CAGGGCCATCAACAAGAACAAGTGCGACATCGACGACCTGAAGATGGCCGTGTCCTTC
AGCCAGTTCAACCGGCGGTTCCTGAATGTCGTGCGGCAGTTCTCTGACAACGCCGGCA
TCACACCAGCCATCAGCCTGGATCTGATGACCGATGCCGAACTGGCTAGAGCCGTGTC
CAACATGCCTACATCTGCCGCCCAGATCAAGCTGATGCTGGAAAACAGAGCCATGGTC
CGACGGAAAGGCTTCGGCATCCTGATCGGCGTGTACGGCAGCAGCGTGATCTACATGG
TGCAGCTGCCTATCTTCGGCGTGATCGACACCCCTTGCTGGATCGTGAAAGCCGCTCC
TAGCTGCAGCGAGAAGAAGGGCAATTACGCCTGCCTGCTGAGAGAGGACCAAGGCTG
GTACTGTCAGAATGCCGGCAGCACCGTGTACTACCCCAACGAGAAGGACTGCGAGACA
AGAGGCGACCACGTGTTCTGTGATACCGCCGCTGGAATCAACGTGGCCGAGCAGAGC
AAAGAGTGCAACATCAACATCAGCACCACAAACTACCCCTGCAAGGTGTCCATCGGCA
GACACCCTATCAGCATGGTGGCTCTGTCTCCACTGGGAGCCCTGGTGGCTTGTTATAA
GGGCGTGTCCTGTAGCATCGGCAGCAACAGAGTGGGCATCATCAAGCAGCTGAACAAG
GGCTGCTCCTACATCTGCAACCAGGACGCCGATACCGTGACCATCGACAATACCGTGT
ATCAGCTGAGCAAGGTGGAAGGCGAATGCCACGTGATCAAGGGCAGACCTGTGTCCAG
CAGCTTCGACCCCGTGAAGTTCCCCGAGGACCAGTTCAATGTGCCCCTGGACCAGGTG
TTCGAGAACATCGAGAACTCTCAGGCTCTGGTGGACCAGTCCAACCGGATTCTGTCTAG
CGCCGAGAAGGGAAACACCGGCTTCATCATCGTGATCATCCTGATTGCCGTGCTGGGC
TCCAGCATGATCCTGGTGTCCATCTTTATCATCATCAAAAAGACGAAGAAGCCCACAGG
CGCCCCTCCAGAACTGTCTGGCGTGACCAACAATGGCTTCATCCCTCACAGC
>hMPV083_mRNA (SEQ ID NO: 399)
AUGAGCUGGAAGGUGGUCAUCAUCUUCAGCCUGCUGAUCACCCCUCAGCACGGCCU
GAAAGAGAGCUACCUGGAAGAAAGCUGCAGCACCAUCACCGAGGGCUACCUGAGCGU
GCUGAGAACCGGCUGGUACACCAACGUGUUCAUCCUGGAAGUGGGCGACGUGGAAA
ACCUGACCUGUGCCGAUGGACCCAGCCUGAUCAAGACCGAGCUGGACCUGACAAAGA
GCGCCCUGCGGGAACUGAAAACCGUGUCUGCAGAUCAGCUGGCCAGAGAGGAACAG
AUCGAGAACCCCAGACAGAGCAGAUUCGUGCUGGGAGCUAUCGCCCUGGGAGUUGC
UACAGCUGCUGCUGUGACAGCUGGCGUGGCCAUUGCCAAGACCAUCCGGCUGGAAA
GCGAAGUGACCGCCAUCAAGAACUGCCUGAAAAAGACCAACGAGGCCGUGUGCACCC
UCGGCAAUGGCGUUAGAGUGCUGGCCACAGCCGUCCGCGAGCUGAAGGAUUUCGUG
UCCAAGAACCUGACCAGGGCCAUCAACAAGAACAAGUGCGACAUCGACGACCUGAAG
AUGGCCGUGUCCUUCAGCCAGUUCAACCGGCGGUUCCUGAAUGUCGUGCGGCAGUU
CUCUGACAACGCCGGCAUCACACCAGCCAUCAGCCUGGAUCUGAUGACCGAUGCCGA
ACUGGCUAGAGCCGUGUCCAACAUGCCUACAUCUGCCGCCCAGAUCAAGCUGAUGC
UGGAAAACAGAGCCAUGGUCCGACGGAAAGGCUUCGGCAUCCUGAUCGGCGUGUAC
GGCAGCAGCGUGAUCUACAUGGUGCAGCUGCCUAUCUUCGGCGUGAUCGACACCCC
UUGCUGGAUCGUGAAAGCCGCUCCUAGCUGCAGCGAGAAGAAGGGCAAUUACGCCU
GCCUGCUGAGAGAGGACCAAGGCUGGUACUGUCAGAAUGCCGGCAGCACCGUGUAC
UACCCCAACGAGAAGGACUGCGAGACAAGAGGCGACCACGUGUUCUGUGAUACCGC
CGCUGGAAUCAACGUGGCCGAGCAGAGCAAAGAGUGCAACAUCAACAUCAGCACCAC
AAACUACCCCUGCAAGGUGUCCAUCGGCAGACACCCUAUCAGCAUGGUGGCUCUGU
CUCCACUGGGAGCCCUGGUGGCUUGUUAUAAGGGCGUGUCCUGUAGCAUCGGCAGC
AACAGAGUGGGCAUCAUCAAGCAGCUGAACAAGGGCUGCUCCUACAUCUGCAACCAG
GACGCCGAUACCGUGACCAUCGACAAUACCGUGUAUCAGCUGAGCAAGGUGGAAGG
CGAAUGCCACGUGAUCAAGGGCAGACCUGUGUCCAGCAGCUUCGACCCCGUGAAGU
UCCCCGAGGACCAGUUCAAUGUGCCCCUGGACCAGGUGUUCGAGAACAUCGAGAAC
UCUCAGGCUCUGGUGGACCAGUCCAACCGGAUUCUGUCUAGCGCCGAGAAGGGAAA
CACCGGCUUCAUCAUCGUGAUCAUCCUGAUUGCCGUGCUGGGCUCCAGCAUGAUCC
UGGUGUCCAUCUUUAUCAUCAUCAAAAAGACGAAGAAGCCCACAGGCGCCCCUCCAG
AACUGUCUGGCGUGACCAACAAUGGCUUCAUCCCUCACAGC
>PIV1_LIT_prefusion_comparison_DNA (SEQ ID NO: 400)
ATGCAGAGCAGCGAGATCCTGCTGCTGGTGTACAGCTCTCTGCTGCTGAGCAGCTCCC
TGTGTCAGATCCCCGTGGACAAGCTGAGCAACGTGGGCGTGATCATCAACGAGGGCAA
GCTGCTGAAGATCGCCGGCAGCTACGAGAGCCGGTACATCGTGCTGTCTCTGGTGCCC
AGCATCGACCTGCAGGATGGCTGTGGCACCACACAGATCATCCAGTACAAGAACCTGC
TGAACCGGCTGCTGATCCCTCTGAAGGATGCCCTGGATCTGCAAGAGAGCCTGATCAC
CATCACCAACGACACCACCGTGACCAACGATAACCCTCAGACCAGAGGCTCCGGCGCC
GTGATCGGAACAATCGCCCTGGGAGTTGCTACCGCCGCTCAGATCACAGCCGGAATTG
CTCTGGCCGAAGCCAGAGAGGCCAGAAAGGATATCGCCCTGATCAAGGACAGCATCGT
GAAAACCCACAACAGCGTGGAATTCATCCAGCGCGGCATCGGCGAGCAGATCATTGCC
CTGAAAACCCTGCAGGACTTCGTGAACGACGAGATCAGACCCGCCATCGGAGAGCTGA
GATGCGAGACAACAGCCCTGAAGCTGGGCATCAAGCTGACCCAGCACTACAGCGAACT
GGCCACCGCCTTTAGCAGCAACCTGGGCACAATCGGCGAGAAGTCCCTGACACTGCAG
GCCCTGAGCAGCCTGTACAGCGCCAATATCACAGAGATCCTGTCCACCATCAAGAAGG
ACAAGAGCGACATCTACGACATCATCTACACCGAGCAAGTGAAGGGCACCGTGATCGA
CGTGGACCTGGAAAAGTACATGGTCACCCTGCTGGTCAAGATCCCCATCCTGTCTGAG
ATCCCTGGCGTGCTGATCTACAGAGCCAGCAGCATCAGCTACAACATCGAGGGCGAAG
AATGGCACGTGGCAATCCCCAACTACATCATCAACAAGGCCAGCAGCCTCGGCGGAGC
CGATGTGACAAATTGCATCGAGAGCAAGCTGGCCTACATCTGCCCCAGAGATCCCACA
CAGCTGATCCCCGACAACCAGCAGAAGTGCATCCTGGGCGACGTGTCCAAGTGTCCTG
TGACCAAAGTGATCAACAACCTGGTGCCTAAGTTCGCCTTCATCAACGGCGGCGTGGT
GGCCAACTGTATCGCCAGCACATGCACATGCGGCACCAACAGAATCCCCGTGAACCAG
GACAGATCCAAGGGCGTGACCTTCCTGACCTACACCAACTGTGGCCTGATCGGCATCA
ATGGCATCGAGCTGTACGCCAACAAGCGGGGCAGAGATACCACCTGGGGCAACCAGA
TCATCAAAGTGGGCCCTGCCGTGTCCATCCGGCCTGTGGATATCTCTCTGAACCTGGC
CAGCATCACCAACTTTCTGGAAGAAATCAAGACCGAACTCATGAAGGCCAGAGCCATCA
TCTCTGCCGTTGGCGGCTGGCACAACAAAGAGAGCACCCAGATCATTATCATCATCATC
GTCTGCATCCTGATCATCATTATCTGCGGCATCCTGTACTACCTGTACAGAGTGCGGAG
ACTGCTCATCATGATCAACAGCACCAACAATAGCCCCATCAACGCCTACACACTGGAAA
GCCGGATGAAGAACCCCTACATGGGCAACCACAGCAAC
>PIV1_LIT_prefusion_comparison_mRNA (SEQ ID NO: 401)
AUGCAGAGCAGCGAGAUCCUGCUGCUGGUGUACAGCUCUCUGCUGCUGAGCAGCUC
CCUGUGUCAGAUCCCCGUGGACAAGCUGAGCAACGUGGGCGUGAUCAUCAACGAGG
GCAAGCUGCUGAAGAUCGCCGGCAGCUACGAGAGCCGGUACAUCGUGCUGUCUCUG
GUGCCCAGCAUCGACCUGCAGGAUGGCUGUGGCACCACACAGAUCAUCCAGUACAA
GAACCUGCUGAACCGGCUGCUGAUCCCUCUGAAGGAUGCCCUGGAUCUGCAAGAGA
GCCUGAUCACCAUCACCAACGACACCACCGUGACCAACGAUAACCCUCAGACCAGAG
GCUCCGGCGCCGUGAUCGGAACAAUCGCCCUGGGAGUUGCUACCGCCGCUCAGAUC
ACAGCCGGAAUUGCUCUGGCCGAAGCCAGAGAGGCCAGAAAGGAUAUCGCCCUGAU
CAAGGACAGCAUCGUGAAAACCCACAACAGCGUGGAAUUCAUCCAGCGCGGCAUCGG
CGAGCAGAUCAUUGCCCUGAAAACCCUGCAGGACUUCGUGAACGACGAGAUCAGACC
CGCCAUCGGAGAGCUGAGAUGCGAGACAACAGCCCUGAAGCUGGGCAUCAAGCUGA
CCCAGCACUACAGCGAACUGGCCACCGCCUUUAGCAGCAACCUGGGCACAAUCGGC
GAGAAGUCCCUGACACUGCAGGCCCUGAGCAGCCUGUACAGCGCCAAUAUCACAGAG
AUCCUGUCCACCAUCAAGAAGGACAAGAGCGACAUCUACGACAUCAUCUACACCGAG
CAAGUGAAGGGCACCGUGAUCGACGUGGACCUGGAAAAGUACAUGGUCACCCUGCU
GGUCAAGAUCCCCAUCCUGUCUGAGAUCCCUGGCGUGCUGAUCUACAGAGCCAGCA
GCAUCAGUACAACAUCGAGGGCGAAGAAUGGCACGUGGCAAUCCCCAACUACAUCA
UCAACAAGGCCAGCAGCCUCGGCGGAGCCGAUGUGACAAAUUGCAUCGAGAGCAAG
CUGGCCUACAUCUGCCCCAGAGAUCCCACACAGCUGAUCCCCGACAACCAGCAGAAG
UGCAUCCUGGGCGACGUGUCCAAGUGUCCUGUGACCAAAGUGAUCAACAACCUGGU
GCCUAAGUUCGCCUUCAUCAACGGCGGCGUGGUGGCCAACUGUAUCGCCAGCACAU
GCACAUGCGGCACCAACAGAAUCCCCGUGAACCAGGACAGAUCCAAGGGCGUGACCU
UCCUGACCUACACCAACUGUGGCCUGAUCGGCAUCAAUGGCAUCGAGCUGUACGCC
AACAAGCGGGGCAGAGAUACCACCUGGGGCAACCAGAUCAUCAAAGUGGGCCCUGC
CGUGUCCAUCCGGCCUGUGGAUAUCUCUCUGAACCUGGCCAGCAUCACCAACUUUC
UGGAAGAAAUCAAGACCGAACUCAUGAAGGCCAGAGCCAUCAUCUCUGCCGUUGGCG
GCUGGCACAACAAAGAGAGCACCCAGAUCAUUAUCAUCAUCAUCGUCUGCAUCCUGA
UCAUCAUUAUCUGCGGCAUCCUGUACUACCUGUACAGAGUGCGGAGACUGCUCAUCA
UGAUCAACAGCACCAACAAUAGCCCCAUCAACGCCUACACACUGGAAAGCCGGAUGA
AGAACCCCUACAUGGGCAACCACAGCAAC
>PIV1047_DNA (SEQ ID NO: 402)
ATGCAGAGCAGCGAGATCCTGCTGCTGGTGTACAGCTCTCTGCTGCTGAGCAGCTCCC
TGTGTCAGATCCCCGTGGACAAGCTGAGCAACGTGGGCGTGATCATCAACGAGGGCAA
GCTGCTGAAGATCGCCGGCAGCTACGAGAGCCGGTACATCGTGCTGTCTCTGGTGCCC
AGCATCGACCTGCAGGATGGCTGTGGCACCACACAGATCATCCAGTACAAGAACCTGC
TGAACCGGCTGCTGATCCCTCTGAAGGATGCCCTGGATCTGTGCGAGAGCCTGATCAC
CATCACCAACGACACCACCGTGACCAACGATAACCCTCAGACCAGAGGCAGCGGCGCC
GTGATCGGAACAATCGCCCTGGGAGTTGCTACCGCCGCTCAGATCACAGCCTGCATTG
CTCTGGCCGAAGCCAGAGAGGCCAGAAAGGATATCGCCCTGATCAAGGACAGCATCGT
GAAAACCCACAACAGCGTGGAATTCATCCAGCGCGGCATCGGCGAGCAGATCATTGCC
CTGAAAACCCTGCAGGACTTCGTGAACGACGAGATCAGACCCGCCATCGGAGAGCTGA
GATGCGAGACAACAGCCCTGAAGCTGGGCATCAAGCTGACCCAGCACTACAGCGAACT
GGCCACCGCCTTTAGCAGCAACCTGGGCACAATCGGCGAGAAGTCCCTGACACTGCAG
GCCCTGAGCAGCCTGTACAGCGCCAATATCACAGAGATCCTGTCCACCATCAAGAAGG
ACAAGAGCGACATCTACGACATCATCTACACCGAGCAAGTGAAGGGCACCGTGATCGA
CGTGGACCTGGAAAAGTACATGGTCACCCTGCTGGTCAAGATCCCCATCCTGTCTGAG
ATCCCTGGCGTGCTGATCTACAGAGCCAGCAGCATCAGCTACAACATCGAGGGCGAAG
AATGGCACGTGGCAATCCCCAACTACATCATCAACAAGGCCAGCAGCCTCGGCGGAGC
CGATGTGACAAATTGCATCGAGAGCAAGCTGGCCTACATCTGCCCCAGAGATCCCACA
CAGCTGATCCCCGACAACCAGCAGAAGTGCATCCTGGGCGACGTGTCCAAGTGTCCTG
TGACCAAAGTGATCAACAACCTGGTGCCTAAGTTCGCCTTCATCAACGGCGGCGTGGT
GGCCAACTGTATCGCCAGCACATGCACATGCGGCACCAACAGAATCCCCGTGAACCAG
GACAGATCCAAGGGCGTGACCTTCCTGACCTACACCAACTGTGGCCTGATCGGCATCA
ATGGCATCGAGCTGTACGCCAACAAGCGGGGCAGAGATACCACCTGGGGCAACCAGA
TCATCAAAGTGGGCCCTGCCGTGTCCATCCGGCCTGTGGATATCTCTCTGAACCTGGC
CAGCCTGACCAACTTTCTGGAAGAACTGAAGACCGAACTCATGAAGCTGAGAGCCATCA
TCTCTGCCGTTGGCGGCTGGCACAACAAAGAGAGCACCCAGATCATTATCATCATCATC
GTCTGCATCCTGATCATCATTATCTGCGGCATCCTGTACTACCTGTACAGAGTGCGGAG
ACTGCTCATCATGATCAACAGCACCAACAATAGCCCCATCAACGCCTACACACTGGAAA
GCCGGATGAAGAACCCCTACATGGGCAACCACAGCAAC
>PIV1047_mRNA (SEQ ID NO: 403)
AUGCAGAGCAGCGAGAUCCUGCUGCUGGUGUACAGCUCUCUGCUGCUGAGCAGCUC
CCUGUGUCAGAUCCCCGUGGACAAGCUGAGCAACGUGGGCGUGAUCAUCAACGAGG
GCAAGCUGCUGAAGAUCGCCGGCAGCUACGAGAGCCGGUACAUCGUGCUGUCUCUG
GUGCCCAGCAUCGACCUGCAGGAUGGCUGUGGCACCACACAGAUCAUCCAGUACAA
GAACCUGCUGAACCGGCUGCUGAUCCCUCUGAAGGAUGCCCUGGAUCUGUGCGAGA
GCCUGAUCACCAUCACCAACGACACCACCGUGACCAACGAUAACCCUCAGACCAGAG
GCAGCGGCGCCGUGAUCGGAACAAUCGCCCUGGGAGUUGCUACCGCCGCUCAGAUC
ACAGCCUGCAUUGCUCUGGCCGAAGCCAGAGAGGCCAGAAAGGAUAUCGCCCUGAU
CAAGGACAGCAUCGUGAAAACCCACAACAGCGUGGAAUUCAUCCAGCGCGGCAUCGG
CGAGCAGAUCAUUGCCCUGAAAACCCUGCAGGACUUCGUGAACGACGAGAUCAGACC
CGCCAUCGGAGAGCUGAGAUGCGAGACAACAGCCCUGAAGCUGGGCAUCAAGCUGA
CCCAGCACUACAGCGAACUGGCCACCGCCUUUAGCAGCAACCUGGGCACAAUCGGC
GAGAAGUCCCUGACACUGCAGGCCCUGAGCAGCCUGUACAGCGCCAAUAUCACAGAG
AUCCUGUCCACCAUCAAGAAGGACAAGAGCGACAUCUACGACAUCAUCUACACCGAG
CAAGUGAAGGGCACCGUGAUCGACGUGGACCUGGAAAAGUACAUGGUCACCCUGCU
GGUCAAGAUCCCCAUCCUGUCUGAGAUCCCUGGCGUGCUGAUCUACAGAGCCAGCA
GCAUCAGCUACAACAUCGAGGGCGAAGAAUGGCACGUGGCAAUCCCCAACUACAUCA
UCAACAAGGCCAGCAGCCUCGGCGGAGCCGAUGUGACAAAUUGCAUCGAGAGCAAG
CUGGCCUACAUCUGCCCCAGAGAUCCCACACAGCUGAUCCCCGACAACCAGCAGAAG
UGCAUCCUGGGCGACGUGUCCAAGUGUCCUGUGACCAAAGUGAUCAACAACCUGGU
GCCUAAGUUCGCCUUCAUCAACGGCGGCGUGGUGGCCAACUGUAUCGCCAGCACAU
GCACAUGCGGCACCAACAGAAUCCCCGUGAACCAGGACAGAUCCAAGGGCGUGACCU
UCCUGACCUACACCAACUGUGGCCUGAUCGGCAUCAAUGGCAUCGAGCUGUACGCC
AACAAGCGGGGCAGAGAUACCACCUGGGGCAACCAGAUCAUCAAAGUGGGCCCUGC
CGUGUCCAUCCGGCCUGUGGAUAUCUCUCUGAACCUGGCCAGCCUGACCAACUUUC
UGGAAGAACUGAAGACCGAACUCAUGAAGCUGAGAGCCAUCAUCUCUGCCGUUGGC
GGCUGGCACAACAAAGAGAGCACCCAGAUCAUUAUCAUCAUCAUCGUCUGCAUCCUG
AUCAUCAUUAUCUGCGGCAUCCUGUACUACCUGUACAGAGUGCGGAGACUGCUCAUC
AUGAUCAACAGCACCAACAAUAGCCCCAUCAACGCCUACACACUGGAAAGCCGGAUG
AAGAACCCCUACAUGGGCAACCACAGCAAC
>PIV1053_DNA (SEQ ID NO: 404)
ATGCAGAGCAGCGAGATCCTGCTGCTGGTGTACAGCTCTCTGCTGCTGAGCAGCTCCC
TGTGTCAGATCCCCGTGGACAAGCTGAGCAACGTGGGCGTGATCATCAACGAGGGCAA
GCTGCTGAAGATCGCCGGCAGCTACGAGAGCCGGTACATCGTGCTGTCTCTGGTGCCC
AGCATCGACCTGCAGGATGGCTGTGGCACCACACAGATCATCCAGTACAAGAACCTGC
TGAACCGGCTGCTGATCCCTCTGAAGGATGCCCTGGATCTGCAAGAGAGCCTGATCAC
CATCACCAACGACACCACCGTGACCAACGATAACCCTCAGACCAGAGGCAGCGGCGCC
GTGATCGGAACAATCGCCCTGGGAGTTGCTACCGCCGCTCAGATCACAGCCGCCATTG
CTCTGGCCGAAGCCAGAGAGGCCAGAAAGGATATCGCCCTGATCAAGGACAGCATCGT
GAAAACCCACAACAGCGTGGAATTCATCCAGCGCGGCATCGGCGAGCAGATCATTGCC
CTGAAAACCCTGCAGGACTTCGTGAACGACGAGATCAGACCCGCCATCGGAGAGCTGA
GATGCGAGACAACAGCCCTGAAGCTGGGCATCAAGCTGACCCAGCACTACAGCGAACT
GGCCACCGCCTTTAGCAGCAACCTGGGCACAATCGGCGAGAAGTCCCTGACACTGCAG
GCCCTGAGCAGCCTGTACAGCGCCAATATCACAGAGATCCTGTCCACCATCAAGAAGG
ACAAGAGCGACATCTACGACATCATCTACACCGAGCAAGTGAAGGGCACCGTGATCGA
CGTGGACCTGGAAAAGTACATGGTCACCCTGCTGGTCAAGATCCCCATCCTGTCTGAG
ATCCCTGGCGTGCTGATCTACAGAGCCAGCAGCATCAGCTACAACATCGAGGGCGAAG
AATGGCACGTGGCAATCCCCAACTACATCATCAACAAGGCCAGCAGCCTCGGCGGAGC
CGATGTGACAAATTGCATCGAGAGCAAGCTGGCCTACATCTGCCCCAGAGATCCCACA
CAGCTGATCCCCGACAACCAGCAGAAGTGCATCCTGGGCGACGTGTCCAAGTGTCCTG
TGACCAAAGTGATCAACAACCTGGTGCCTAAGTTCGCCTTCATCAACGGCGGCGTGGT
GGCCAACTGTATCGCCAGCACATGCACATGCGGCACCAACAGAATCCCCGTGAACCAG
GACAGATCCAAGGGCGTGACCTTCCTGACCTACACCAACTGTGGCCTGATCGGCATCA
ATGGCATCGAGCTGTACGCCAACAAGCGGGGCAGAGATACCACCTGGGGCAACCAGA
TCATCAAAGTGGGCCCTGCCGTGTCCATCCGGCCTGTGGATATCTCTCTGAACCTGGC
CAGCCTGACCAACTTTCTGGAAGAACTGAAGACCGAACTCATGAAGCTGAGAGCCATCA
TCTCTGCCGTTGGCGGCTGGCACAACAAAGAGAGCACCCAGATCATTATCATCATCATC
GTCTGCATCCTGATCATCATTATCTGCGGCATCCTGTACTACCTGTACAGAGTGCGGAG
ACTGCTCATCATGATCAACAGCACCAACAATAGCCCCATCAACGCCTACACACTGGAAA
GCCGGATGAAGAACCCCTACATGGGCAACCACAGCAAC
>PIV1053_mRNA (SEQ ID NO: 405)
AUGCAGAGCAGCGAGAUCCUGCUGCUGGUGUACAGCUCUCUGCUGCUGAGCAGCUC
CCUGUGUCAGAUCCCCGUGGACAAGCUGAGCAACGUGGGCGUGAUCAUCAACGAGG
GCAAGCUGCUGAAGAUCGCCGGCAGCUACGAGAGCCGGUACAUCGUGCUGUCUCUG
GUGCCCAGCAUCGACCUGCAGGAUGGCUGUGGCACCACACAGAUCAUCCAGUACAA
GAACCUGCUGAACCGGCUGCUGAUCCCUCUGAAGGAUGCCCUGGAUCUGCAAGAGA
GCCUGAUCACCAUCACCAACGACACCACCGUGACCAACGAUAACCCUCAGACCAGAG
GCAGCGGCGCCGUGAUCGGAACAAUCGCCCUGGGAGUUGCUACCGCCGCUCAGAUC
ACAGCCGCCAUUGCUCUGGCCGAAGCCAGAGAGGCCAGAAAGGAUAUCGCCCUGAU
CAAGGACAGCAUCGUGAAAACCCACAACAGCGUGGAAUUCAUCCAGCGCGGCAUCGG
CGAGCAGAUCAUUGCCCUGAAAACCCUGCAGGACUUCGUGAACGACGAGAUCAGACC
CGCCAUCGGAGAGCUGAGAUGCGAGACAACAGCCCUGAAGCUGGGCAUCAAGCUGA
CCCAGCACUACAGCGAACUGGCCACCGCCUUUAGCAGCAACCUGGGCACAAUCGGC
GAGAAGUCCCUGACACUGCAGGCCCUGAGCAGCCUGUACAGCGCCAAUAUCACAGAG
AUCCUGUCCACCAUCAAGAAGGACAAGAGCGACAUCUACGACAUCAUCUACACCGAG
CAAGUGAAGGGCACCGUGAUCGACGUGGACCUGGAAAAGUACAUGGUCACCCUGCU
GGUCAAGAUCCCCAUCCUGUCUGAGAUCCCUGGCGUGCUGAUCUACAGAGCCAGCA
GCAUCAGCUACAACAUCGAGGGCGAAGAAUGGCACGUGGCAAUCCCCAACUACAUCA
UCAACAAGGCCAGCAGCCUCGGCGGAGCCGAUGUGACAAAUUGCAUCGAGAGCAAG
CUGGCCUACAUCUGCCCCAGAGAUCCCACACAGCUGAUCCCCGACAACCAGCAGAAG
UGCAUCCUGGGCGACGUGUCCAAGUGUCCUGUGACCAAAGUGAUCAACAACCUGGU
GCCUAAGUUCGCCUUCAUCAACGGCGGCGUGGUGGCCAACUGUAUCGCCAGCACAU
GCACAUGCGGCACCAACAGAAUCCCCGUGAACCAGGACAGAUCCAAGGGCGUGACCU
UCCUGACCUACACCAACUGUGGCCUGAUCGGCAUCAAUGGCAUCGAGCUGUACGCC
AACAAGCGGGGCAGAGAUACCACCUGGGGCAACCAGAUCAUCAAAGUGGGCCCUGC
CGUGUCCAUCCGGCCUGUGGAUAUCUCUCUGAACCUGGCCAGCCUGACCAACUUUC
UGGAAGAACUGAAGACCGAACUCAUGAAGCUGAGAGCCAUCAUCUCUGCCGUUGGC
GGCUGGCACAACAAAGAGAGCACCCAGAUCAUUAUCAUCAUCAUCGUCUGCAUCCUG
AUCAUCAUUAUCUGCGGCAUCCUGUACUACCUGUACAGAGUGCGGAGACUGCUCAUC
AUGAUCAACAGCACCAACAAUAGCCCCAUCAACGCCUACACACUGGAAAGCCGGAUG
AAGAACCCCUACAUGGGCAACCACAGCAAC
>PIV1054_DNA (SEQ ID NO: 406)
ATGCAGAGCAGCGAGATCCTGCTGCTGGTGTACAGCTCTCTGCTGCTGAGCAGCTCCC
TGTGTCAGATCCCCGTGGACAAGCTGAGCAACGTGGGCGTGATCATCAACGAGGGCAA
GCTGCTGAAGATCGCCGGCAGCTACGAGAGCCGGTACATCGTGCTGTCTCTGGTGCCC
AGCATCGACCTGCAGGATGGCTGTGGCACCACACAGATCATCCAGTACAAGAACCTGC
TGAACCGGCTGCTGATCCCTCTGAAGGATGCCCTGGATCTGCAAGAGAGCCTGATCAC
CATCACCAACGACACCACCGTGACCAACGATAACCCTCAGACCAGATTCTTCGGCGCC
GTGATCGGAACAATCGCCCTGGGAGTTGCTACCGCCGCTCAGATCACAGCCGCCATTG
CTCTGGCCGAAGCCAGAGAGGCCAGAAAGGATATCGCCCTGATCAAGGACAGCATCGT
GAAAACCCACAACAGCGTGGAATTCATCCAGCGCGGCATCGGCGAGCAGATCATTGCC
CTGAAAACCCTGCAGGACTTCGTGAACGACGAGATCAGACCCGCCATCGGAGAGCTGA
GATGCGAGACAACAGCCCTGAAGCTGGGCATCAAGCTGACCCAGCACTACAGCGAACT
GGCCACCGCCTTTAGCAGCAACCTGGGCACAATCGGCGAGAAGTCCCTGACACTGCAG
GCCCTGAGCAGCCTGTACAGCGCCAATATCACAGAGATCCTGTCCACCATCAAGAAGG
ACAAGAGCGACATCTACGACATCATCTACACCGAGCAAGTGAAGGGCACCGTGATCGA
CGTGGACCTGGAAAAGTACATGGTCACCCTGCTGGTCAAGATCCCCATCCTGTCTGAG
ATCCCTGGCGTGCTGATCTACAGAGCCAGCAGCATCAGCTACAACATCGAGGGCGAAG
AATGGCACGTGGCAATCCCCAACTACATCATCAACAAGGCCAGCAGCCTCGGCGGAGC
CGATGTGACAAATTGCATCGAGAGCAAGCTGGCCTACATCTGCCCCAGAGATCCCACA
CAGCTGATCCCCGACAACCAGCAGAAGTGCATCCTGGGCGACGTGTCCAAGTGTCCTG
TGACCAAAGTGATCAACAACCTGGTGCCTAAGTTCGCCTTCATCAACGGCGGCGTGGT
GGCCAACTGTATCGCCAGCACATGCACATGCGGCACCAACAGAATCCCCGTGAACCAG
GACAGATCCAAGGGCGTGACCTTCCTGACCTACACCAACTGTGGCCTGATCGGCATCA
ATGGCATCGAGCTGTACGCCAACAAGCGGGGCAGAGATACCACCTGGGGCAACCAGA
TCATCAAAGTGGGCCCTGCCGTGTCCATCCGGCCTGTGGATATCTCTCTGAACCTGGC
CAGCCTGACCAACTTTCTGGAAGAACTGAAGACCGAACTCATGAAGCTGAGAGCCATCA
TCTCTGCCGTTGGCGGCTGGCACAACAAAGAGAGCACCCAGATCATTATCATCATCATC
GTCTGCATCCTGATCATCATTATCTGCGGCATCCTGTACTACCTGTACAGAGTGCGGAG
ACTGCTCATCATGATCAACAGCACCAACAATAGCCCCATCAACGCCTACACACTGGAAA
GCCGGATGAAGAACCCCTACATGGGCAACCACAGCAAC
>PIV1054_mRNA (SEQ ID NO: 407)
AUGCAGAGCAGCGAGAUCCUGCUGCUGGUGUACAGCUCUCUGCUGCUGAGCAGCUC
CCUGUGUCAGAUCCCCGUGGACAAGCUGAGCAACGUGGGCGUGAUCAUCAACGAGG
GCAAGCUGCUGAAGAUCGCCGGCAGCUACGAGAGCCGGUACAUCGUGCUGUCUCUG
GUGCCCAGCAUCGACCUGCAGGAUGGCUGUGGCACCACACAGAUCAUCCAGUACAA
GAACCUGCUGAACCGGCUGCUGAUCCCUCUGAAGGAUGCCCUGGAUCUGCAAGAGA
GCCUGAUCACCAUCACCAACGACACCACCGUGACCAACGAUAACCCUCAGACCAGAU
UCUUCGGCGCCGUGAUCGGAACAAUCGCCCUGGGAGUUGCUACCGCCGCUCAGAUC
ACAGCCGCCAUUGCUCUGGCCGAAGCCAGAGAGGCCAGAAAGGAUAUCGCCCUGAU
CAAGGACAGCAUCGUGAAAACCCACAACAGCGUGGAAUUCAUCCAGCGCGGCAUCGG
CGAGCAGAUCAUUGCCCUGAAAACCCUGCAGGACUUCGUGAACGACGAGAUCAGACC
CGCCAUCGGAGAGCUGAGAUGCGAGACAACAGCCCUGAAGCUGGGCAUCAAGCUGA
CCCAGCACUACAGCGAACUGGCCACCGCCUUUAGCAGCAACCUGGGCACAAUCGGC
GAGAAGUCCCUGACACUGCAGGCCCUGAGCAGCCUGUACAGCGCCAAUAUCACAGAG
AUCCUGUCCACCAUCAAGAAGGACAAGAGCGACAUCUACGACAUCAUCUACACCGAG
CAAGUGAAGGGCACCGUGAUCGACGUGGACCUGGAAAAGUACAUGGUCACCCUGCU
GGUCAAGAUCCCCAUCCUGUCUGAGAUCCCUGGCGUGCUGAUCUACAGAGCCAGCA
GCAUCAGCUACAACAUCGAGGGCGAAGAAUGGCACGUGGCAAUCCCCAACUACAUCA
UCAACAAGGCCAGCAGCCUCGGCGGAGCCGAUGUGACAAAUUGCAUCGAGAGCAAG
CUGGCCUACAUCUGCCCCAGAGAUCCCACACAGCUGAUCCCCGACAACCAGCAGAAG
UGCAUCCUGGGCGACGUGUCCAAGUGUCCUGUGACCAAAGUGAUCAACAACCUGGU
GCCUAAGUUCGCCUUCAUCAACGGCGGCGUGGUGGCCAACUGUAUCGCCAGCACAU
GCACAUGCGGCACCAACAGAAUCCCCGUGAACCAGGACAGAUCCAAGGGCGUGACCU
UCCUGACCUACACCAACUGUGGCCUGAUCGGCAUCAAUGGCAUCGAGCUGUACGCC
AACAAGCGGGGCAGAGAUACCACCUGGGGCAACCAGAUCAUCAAAGUGGGCCCUGC
CGUGUCCAUCCGGCCUGUGGAUAUCUCUCUGAACCUGGCCAGCCUGACCAACUUUC
UGGAAGAACUGAAGACCGAACUCAUGAAGCUGAGAGCCAUCAUCUCUGCCGUUGGC
GGCUGGCACAACAAAGAGAGCACCCAGAUCAUUAUCAUCAUCAUCGUCUGCAUCCUG
AUCAUCAUUAUCUGCGGCAUCCUGUACUACCUGUACAGAGUGCGGAGACUGCUCAUC
AUGAUCAACAGCACCAACAAUAGCCCCAUCAACGCCUACACACUGGAAAGCCGGAUG
AAGAACCCCUACAUGGGCAACCACAGCAAC
>PIV1069_DNA (SEQ ID NO: 408)
ATGCAGAGCAGCGAGATCCTGCTGCTGGTGTACAGCTCTCTGCTGCTGAGCAGCTCCC
TGTGTCAGATCCCCGTGGACAAGCTGAGCAACGTGGGCGTGATCATCAACGAGGGCAA
GCTGCTGAAGATCGCCGGCAGCTACGAGAGCCGGTACATCGTGCTGTCTCTGGTGCCC
AGCATCGACCTGCAGGATGGCTGTGGCACCACACAGATCATCCAGTACAAGAACCTGC
TGAACCGGCTGCTGATCCCTCTGAAGGATGCCCTGGATCTGCAAGAGAGCCTGATCAC
CATCACCAACGACACCACCGTGACCAACGATAACCCTCAGACCAGAGGCAGCGGCGCC
GTGATCGGAACAATCGCCCTGGGAGTTGCTACCGCCGCTCAGATCACAGCCGCCATTG
CTCTGGCCGAAGCCAGAGAGGCCAGAAAGGATATCGCCCTGATCAAGGACAGCATCGT
GAAAACCCACAACAGCGTGGAATTCATCCAGCGCGGCATCGGCGAGCAGATCATTGCC
CTGAAAACCCTGCAGGACTTCGTGAACGACGAGATCAGACCCGCCATCGGAGAGCTGA
GATGCGAGACAACAGCCCTGAAGCTGGGCATCAAGCTGACCCAGCACTACAGCGAACT
GGCCACCGCCTTTAGCAGCAACCTGGGCACAATCGGCGAGAAGTCCCTGACACTGCAG
GCCCTGAGCAGCCTGTACAGCGCCAATATCACAGAGATCCTGTCCACCATCAAGAAGG
ACAAGAGCGACATCTACGACATCATCTACACCGAGCAAGTGAAGGGCACCGTGATCGA
CGTGGACCTGGAAAAGTACATGGTCACCCTGCTGGTCAAGATCCCCATCCTGTCTGAG
ATCCCTGGCGTGCTGATCTACAGAGCCAGCAGCATCAGCTACAACATCGAGGGCGAAG
AATGGCACGTGGCAATCCCCAACTACATCATCAACAAGGCCAGCAGCCTCGGCGGAGC
CGATGTGACAAATTGCATCGAGAGCAAGCTGGCCTACATCTGCCCCAGAGATCCCACA
CAGCTGATCCCCGACAACCAGCAGAAGTGCATCCTGGGCGACGTGTCCAAGTGTCCTG
TGACCAAAGTGATCAACAACCTGGTGCCTAAGTTCGCCTTCATCAACGGCGGCGTGGT
GGCCAACTGTATCGCCAGCACATGCACATGCGGCACCAACAGAATCCCCGTGAACCAG
GACAGATCCAAGGGCGTGACCTTCCTGACCTACACCAACTGTGGCCTGATCGGCATCA
ATGGCATCGAGCTGTACGCCAACAAGCGGGGCAGAGATACCACCTGGGGCAACCAGA
TCATCAAAGTGGGCCCTGCCGTGTCCATCCGGCCTGTGGATATCTCTCTGAACCTGGC
CAGCCTGACCAACTTTCTGGAAGAACTGAAGACCGAACTCATGAAGGCCAGAGCCATC
ATCTCTGCCGTTGGCGGCTGGCACAACAAAGAGAGCACCCAGATCATTATCATCATCAT
CGTCTGCATCCTGATCATCATTATCTGCGGCATCCTGTACTACCTGTACAGAGTGCGGA
GACTGCTCATCATGATCAACAGCACCAACAATAGCCCCATCAACGCCTACACACTGGAA
AGCCGGATGAAGAACCCCTACATGGGCAACCACAGCAAC
>PIV1069_mRNA (SEQ ID NO: 409)
AUGCAGAGCAGCGAGAUCCUGCUGCUGGUGUACAGCUCUCUGCUGCUGAGCAGCUC
CCUGUGUCAGAUCCCCGUGGACAAGCUGAGCAACGUGGGCGUGAUCAUCAACGAGG
GCAAGCUGCUGAAGAUCGCCGGCAGCUACGAGAGCCGGUACAUCGUGCUGUCUCUG
GUGCCCAGCAUCGACCUGCAGGAUGGCUGUGGCACCACACAGAUCAUCCAGUACAA
GAACCUGCUGAACCGGCUGCUGAUCCCUCUGAAGGAUGCCCUGGAUCUGCAAGAGA
GCCUGAUCACCAUCACCAACGACACCACCGUGACCAACGAUAACCCUCAGACCAGAG
GCAGCGGCGCCGUGAUCGGAACAAUCGCCCUGGGAGUUGCUACCGCCGCUCAGAUC
ACAGCCGCCAUUGCUCUGGCCGAAGCCAGAGAGGCCAGAAAGGAUAUCGCCCUGAU
CAAGGACAGCAUCGUGAAAACCCACAACAGCGUGGAAUUCAUCCAGCGCGGCAUCGG
CGAGCAGAUCAUUGCCCUGAAAACCCUGCAGGACUUCGUGAACGACGAGAUCAGACC
CGCCAUCGGAGAGCUGAGAUGCGAGACAACAGCCCUGAAGCUGGGCAUCAAGCUGA
CCCAGCACUACAGCGAACUGGCCACCGCCUUUAGCAGCAACCUGGGCACAAUCGGC
GAGAAGUCCCUGACACUGCAGGCCCUGAGCAGCCUGUACAGCGCCAAUAUCACAGAG
AUCCUGUCCACCAUCAAGAAGGACAAGAGCGACAUCUACGACAUCAUCUACACCGAG
CAAGUGAAGGGCACCGUGAUCGACGUGGACCUGGAAAAGUACAUGGUCACCCUGCU
GGUCAAGAUCCCCAUCCUGUCUGAGAUCCCUGGCGUGCUGAUCUACAGAGCCAGCA
GCAUCAGCUACAACAUCGAGGGCGAAGAAUGGCACGUGGCAAUCCCCAACUACAUCA
UCAACAAGGCCAGCAGCCUCGGCGGAGCCGAUGUGACAAAUUGCAUCGAGAGCAAG
CUGGCCUACAUCUGCCCCAGAGAUCCCACACAGCUGAUCCCCGACAACCAGCAGAAG
UGCAUCCUGGGCGACGUGUCCAAGUGUCCUGUGACCAAAGUGAUCAACAACCUGGU
GCCUAAGUUCGCCUUCAUCAACGGCGGCGUGGUGGCCAACUGUAUCGCCAGCACAU
GCACAUGCGGCACCAACAGAAUCCCCGUGAACCAGGACAGAUCCAAGGGCGUGACCU
UCCUGACCUACACCAACUGUGGCCUGAUCGGCAUCAAUGGCAUCGAGCUGUACGCC
AACAAGCGGGGCAGAGAUACCACCUGGGGCAACCAGAUCAUCAAAGUGGGCCCUGC
CGUGUCCAUCCGGCCUGUGGAUAUCUCUCUGAACCUGGCCAGCCUGACCAACUUUC
UGGAAGAACUGAAGACCGAACUCAUGAAGGCCAGAGCCAUCAUCUCUGCCGUUGGC
GGCUGGCACAACAAAGAGAGCACCCAGAUCAUUAUCAUCAUCAUCGUCUGCAUCCUG
AUCAUCAUUAUCUGCGGCAUCCUGUACUACCUGUACAGAGUGCGGAGACUGCUCAUC
AUGAUCAACAGCACCAACAAUAGCCCCAUCAACGCCUACACACUGGAAAGCCGGAUG
AAGAACCCCUACAUGGGCAACCACAGCAAC
>PIV3109_DNA (SEQ ID NO: 410)
ATGCTGATCTCCATCCTGCTGATCATCACCACAATGATCATGGCCAGCCACTGCCAGAT
CGACATCACCAAGCTGCAGCACGTGGGCGTGCTGGTCAATAGCCCTAAGGGCATGAAG
ATCAGCCAGAACTTCGAGACACGCTACCTGATCCTGTCTCTGATCCCCAAGATCGAGGA
CAGCAACAGCTGCGGCGACCAGCAGATCAAGCAGTACAAGCGGCTGCTGGACAGACT
GATCATCCCTCTGTACGACGGCCTGCGGCTGCAGAAAGATGTGATCGTGACCAATCAA
GAGAGCAACGAGAACACAGACCCCAGAACCGAGAGATTCTTCGGCGGCGTGATCGGC
ACAATCGCCCTGGGAGTTGCTACAAGCGCCCAGATTACAGCCGCCGTGGCTCTGGTGG
AAGCCAAGCAGGCCAGAAGCGACATCGAGAAGCTGAAAGAGGCCATCCGGGACACCA
ACAAGGCCGTGCAGTGCGTGCAAAGCAGCGTGGGCAATCTGATCTGCGCCATTAAGAG
CGTGCAGGACTACGTGAACAAAGAGATCGTCCCCTCTATCGCCAGACTGGGATGTGAA
GCTGCCGGACTGCAGCTGGGAATTGCCCTGACACAGCACTACAGCGAGCTGACCAACA
TCTTCGGCGACAACATCGGCAGCCTGCAAGAGAAGGGCATTAAGCTGCAGGGAATCGC
CAGCCTGTACCGCACCAACATCACCGAGATCTTCACCACCAGCACCGTGGATAAGTAC
GACATCTACGACCTGCTGTTCACCGAGAGCATCAAAGTGCGCGTGATCGACGTGGACC
TGAACGACTACAGCATCACCCTGCAAGTGCGGCTGCCTCTGCTGACCAGACTGCTGAA
CACCCAGATCTACAAGGTGGACAGCATCTCCTACAACATCCAGAACCGCGAGTGGTAC
ATCCCACTGCCTAGCCACATTATGACCAAGGGCGCCTTTCTCGGCGGAGCCGATGTGA
AAGAGTGCATCGAGGCCTTCAGCAGCTACATCTGCCCTAGCGATCCTGGCTTCGTGCT
GAACCACGAGATGGAAAGCTGCCTGAGCGGCAACATCTCTCAGTGCCCTAGAACCACC
GTGACCTCCGACATCGTGCCCAGATACGCCTTTGTGAATGGCGGCGTGGTGGCCAACT
GCATCACCACCACCTGTACCTGCAACGGCATCGGCAACCGGATCAACCAGCCTCCAGA
TCAGGGCGTGAAGATTATCACCCACAAAGAGTGTAACACCATCGGCATCAACGGCATG
CTGTTCAATACCAACAAAGAGGGCACCCTGGCCTTCTACACCCCTGACGATATCACCCT
GAACAACAGCGTGGCCCTGGATCCTATCGACATCTCCATCGAGCTGAACAAGGCCAAG
AGCGACCTGGAAGAAGCCAAAGAGTGGATCCGGCGGGCCAACCAGAAGCTGGATAGC
ATCGGAAGCTGGCACCAGAGCAGCACCACCATCATCGTGATCCTGATTATGATGATTAT
CCTGTTCATCATCAACATTACCATCATCACGATCGCCATCAAGTACTACCGGATCCAGAA
ACGGAACCGCGTGGACCAGAATGACAAGCCCTACGTGCTGACAAACAAG
>PIV3109_mRNA (SEQ ID NO: 411)
AUGCUGAUCUCCAUCCUGCUGAUCAUCACCACAAUGAUCAUGGCCAGCCACUGCCAG
AUCGACAUCACCAAGCUGCAGCACGUGGGCGUGCUGGUCAAUAGCCCUAAGGGCAU
GAAGAUCAGCCAGAACUUCGAGACACGCUACCUGAUCCUGUCUCUGAUCCCCAAGAU
CGAGGACAGCAACAGCUGCGGCGACCAGCAGAUCAAGCAGUACAAGCGGCUGCUGG
ACAGACUGAUCAUCCCUCUGUACGACGGCCUGCGGCUGCAGAAAGAUGUGAUCGUG
ACCAAUCAAGAGAGCAACGAGAACACAGACCCCAGAACCGAGAGAUUCUUCGGCGGC
GUGAUCGGCACAAUCGCCCUGGGAGUUGCUACAAGCGCCCAGAUUACAGCCGCCGU
GGCUCUGGUGGAAGCCAAGCAGGCCAGAAGCGACAUCGAGAAGCUGAAAGAGGCCA
UCCGGGACACCAACAAGGCCGUGCAGUGCGUGCAAAGCAGCGUGGGCAAUCUGAUC
UGCGCCAUUAAGAGCGUGCAGGACUACGUGAACAAAGAGAUCGUCCCCUCUAUCGCC
AGACUGGGAUGUGAAGCUGCCGGACUGCAGCUGGGAAUUGCCCUGACACAGCACUA
CAGCGAGCUGACCAACAUCUUCGGCGACAACAUCGGCAGCCUGCAAGAGAAGGGCAU
UAAGCUGCAGGGAAUCGCCAGCCUGUACCGCACCAACAUCACCGAGAUCUUCACCAC
CAGCACCGUGGAUAAGUACGACAUCUACGACCUGCUGUUCACCGAGAGCAUCAAAGU
GCGCGUGAUCGACGUGGACCUGAACGACUACAGCAUCACCCUGCAAGUGCGGCUGC
CUCUGCUGACCAGACUGCUGAACACCCAGAUCUACAAGGUGGACAGCAUCUCCUACA
ACAUCCAGAACCGCGAGUGGUACAUCCCACUGCCUAGCCACAUUAUGACCAAGGGCG
CCUUUCUCGGCGGAGCCGAUGUGAAAGAGUGCAUCGAGGCCUUCAGCAGCUACAUC
UGCCCUAGCGAUCCUGGCUUCGUGCUGAACCACGAGAUGGAAAGCUGCCUGAGCGG
CAACAUCUCUCAGUGCCCUAGAACCACCGUGACCUCCGACAUCGUGCCCAGAUACGC
CUUUGUGAAUGGCGGCGUGGUGGCCAACUGCAUCACCACCACCUGUACCUGCAACG
GCAUCGGCAACCGGAUCAACCAGCCUCCAGAUCAGGGCGUGAAGAUUAUCACCCACA
AAGAGUGUAACACCAUCGGCAUCAACGGCAUGCUGUUCAAUACCAACAAAGAGGGCA
CCCUGGCCUUCUACACCCCUGACGAUAUCACCCUGAACAACAGCGUGGCCCUGGAUC
CUAUCGACAUCUCCAUCGAGCUGAACAAGGCCAAGAGCGACCUGGAAGAAGCCAAAG
AGUGGAUCCGGCGGGCCAACCAGAAGCUGGAUAGCAUCGGAAGCUGGCACCAGAGC
AGCACCACCAUCAUCGUGAUCCUGAUUAUGAUGAUUAUCCUGUUCAUCAUCAACAUU
ACCAUCAUCACGAUCGCCAUCAAGUACUACCGGAUCCAGAAACGGAACCGCGUGGAC
CAGAAUGACAAGCCCUACGUGCUGACAAACAAG
>PIV3110_DNA (SEQ ID NO: 412)
ATGCTGATCTCCATCCTGCTGATCATCACCACAATGATCATGGCCAGCCACTGCCAGAT
CGACATCACCAAGCTGCAGCACGTGGGCGTGCTGGTCAATAGCCCTAAGGGCATGAAG
ATCAGCCAGAACTTCGAGACACGCTACCTGATCCTGTCTCTGATCCCCAAGATCGAGGA
CAGCAACAGCTGCGGCGACCAGCAGATCAAGCAGTACAAGCGGCTGCTGGACAGACT
GATCATCCCTCTGTACGACGGCCTGCGGCTGCAGAAAGATGTGATCGTGACCAATCAA
GAGAGCAACGAGAACACAGACCCCAGAACCGAGAGATTCTTCGGCGGCGTGATCGGC
ACAATCGCCCTGGGAGTTGCTACAAGCGCCCAGATTACAGCCGCCGTGGCTCTGGTGG
AAGCCAAGCAGGCCAGAAGCGACATCGAGAAGCTGAAAGAGGCCATCCGGGACACCA
ACAAGGCCGTGCAGTCTGTGCAAAGCAGCGTGGGCAATCTGATCGTGGCCATTAAGAG
CGTGCAGGACTACGTGAACAAAGAGATCGTCCCCTCTATCGCCAGACTGGGATGTGAA
GCTGCCGGACTGCAGCTGGGAATTGCCCTGACACAGCACTACAGCGAGCTGACCAACA
TCTTCGGCGACAACATCGGCAGCCTGCAAGAGAAGGGCATTAAGCTGCAGGCCATCGC
CAGCCTGTACCGCACCAACATCACCGAGATCTTCACCACCAGCACCGTGGATAAGTAC
GACATCTACGACCTGCTGTTCACCGAGAGCATCAAAGTGCGCGTGATCGACGTGGACC
TGAACGACTACAGCATCACCCTGCAAGTGCGGCTGCCTCTGCTGACCAGACTGCTGAA
CACCCAGATCTACAAGGTGGACAGCATCTCCTACAACATCCAGAACCGCGAGTGGTAC
ATCCCACTGCCTAGCCACATTATGACCAAGGGCGCCTTTCTCGGCGGAGCCGATGTGA
AAGAGTGCATCGAGGCCTTCAGCAGCTACATCTGCCCTAGCGATCCTGGCTTCGTGCT
GAACCACGAGATGGAAAGCTGCCTGAGCGGCAACATCTCTCAGTGCCCTAGAACCACC
GTGACCTCCGACATCGTGCCCAGATACGCCTTTGTGAATGGCGGCGTGGTGGCCAACT
GCATCACCACCACCTGTACCTGCAACGGCATCGGCAACCGGATCAACCAGCCTCCAGA
TCAGGGCGTGAAGATTATCACCCACAAAGAGTGTAACACCATCGGCATCAACGGCATG
CTGTTCAATACCAACAAAGAGGGCACCCTGGCCTTCTACACCCCTGACGATATCACCCT
GAACAACAGCGTGGCCCTGGATCCTATCGACATCTCCATCGAGCTGAACAAGGCCAAG
AGCGACCTGGAAGAAGCCAAAGAGTGGATCCGGCGGGCCAACCAGAAGCTGGATAGC
ATCGGAAGCTGGCACCAGAGCAGCACCACCATCATCGTGATCCTGATTATGATGATTAT
CCTGTTCATCATCAACATTACCATCATCACGATCGCCATCAAGTACTACCGGATCCAGAA
ACGGAACCGCGTGGACCAGAATGACAAGCCCTACGTGCTGACAAACAAG
>PIV3110_mRNA (SEQ ID NO: 413)
AUGCUGAUCUCCAUCCUGCUGAUCAUCACCACAAUGAUCAUGGCCAGCCACUGCCAG
AUCGACAUCACCAAGCUGCAGCACGUGGGCGUGCUGGUCAAUAGCCCUAAGGGCAU
GAAGAUCAGCCAGAACUUCGAGACACGCUACCUGAUCCUGUCUCUGAUCCCCAAGAU
CGAGGACAGCAACAGCUGCGGCGACCAGCAGAUCAAGCAGUACAAGCGGCUGCUGG
ACAGACUGAUCAUCCCUCUGUACGACGGCCUGCGGCUGCAGAAAGAUGUGAUCGUG
ACCAAUCAAGAGAGCAACGAGAACACAGACCCCAGAACCGAGAGAUUCUUCGGCGGC
GUGAUCGGCACAAUCGCCCUGGGAGUUGCUACAAGCGCCCAGAUUACAGCCGCCGU
GGCUCUGGUGGAAGCCAAGCAGGCCAGAAGCGACAUCGAGAAGCUGAAAGAGGCCA
UCCGGGACACCAACAAGGCCGUGCAGUCUGUGCAAAGCAGCGUGGGCAAUCUGAUC
GUGGCCAUUAAGAGCGUGCAGGACUACGUGAACAAAGAGAUCGUCCCCUCUAUCGC
CAGACUGGGAUGUGAAGCUGCCGGACUGCAGCUGGGAAUUGCCCUGACACAGCACU
ACAGCGAGCUGACCAACAUCUUCGGCGACAACAUCGGCAGCCUGCAAGAGAAGGGCA
UUAAGCUGCAGGCCAUCGCCAGCCUGUACCGCACCAACAUCACCGAGAUCUUCACCA
CCAGCACCGUGGAUAAGUACGACAUCUACGACCUGCUGUUCACCGAGAGCAUCAAAG
UGCGCGUGAUCGACGUGGACCUGAACGACUACAGCAUCACCCUGCAAGUGCGGCUG
CCUCUGCUGACCAGACUGCUGAACACCCAGAUCUACAAGGUGGACAGCAUCUCCUAC
AACAUCCAGAACCGCGAGUGGUACAUCCCACUGCCUAGCCACAUUAUGACCAAGGGC
GCCUUUCUCGGCGGAGCCGAUGUGAAAGAGUGCAUCGAGGCCUUCAGCAGCUACAU
CUGCCCUAGCGAUCCUGGCUUCGUGCUGAACCACGAGAUGGAAAGCUGCCUGAGCG
GCAACAUCUCUCAGUGCCCUAGAACCACCGUGACCUCCGACAUCGUGCCCAGAUACG
CCUUUGUGAAUGGCGGCGUGGUGGCCAACUGCAUCACCACCACCUGUACCUGCAAC
GGCAUCGGCAACCGGAUCAACCAGCCUCCAGAUCAGGGCGUGAAGAUUAUCACCCAC
AAAGAGUGUAACACCAUCGGCAUCAACGGCAUGCUGUUCAAUACCAACAAAGAGGGC
ACCCUGGCCUUCUACACCCCUGACGAUAUCACCCUGAACAACAGCGUGGCCCUGGAU
CCUAUCGACAUCUCCAUCGAGCUGAACAAGGCCAAGAGCGACCUGGAAGAAGCCAAA
GAGUGGAUCCGGCGGGCCAACCAGAAGCUGGAUAGCAUCGGAAGCUGGCACCAGAG
CAGCACCACCAUCAUCGUGAUCCUGAUUAUGAUGAUUAUCCUGUUCAUCAUCAACAU
UACCAUCAUCACGAUCGCCAUCAAGUACUACCGGAUCCAGAAACGGAACCGCGUGGA
CCAGAAUGACAAGCCCUACGUGCUGACAAACAAG
>PIV3117_DNA (SEQ ID NO: 414)
ATGCTGATCTCCATCCTGCTGATCATCACCACAATGATCATGGCCAGCCACTGCCAGAT
CGACATCACCAAGCTGCAGCACGTGGGCGTGCTGGTCAATAGCCCTAAGGGCATGAAG
ATCAGCCAGAACTTCGAGACACGCTACCTGATCCTGTCTCTGATCCCCAAGATCGAGGA
CAGCAACAGCTGCGGCGACCAGCAGATCAAGCAGTACAAGCGGCTGCTGGACAGACT
GATCATCCCTCTGTACGACGGCCTGCGGCTGCAGAAAGATGTGATCGTGACCAATCAA
GAGAGCAACGAGAACACAGACCCCAGAACCGAGAGATTCTTCGGCGGCGTGATCGGC
ACAATCGCCCTGGGAGTTGCTACAAGCGCCCAGATTACAGCCGCCGTGGCTCTGGTGG
AAGCCAAGCAGGCCAGAAGCGACATCGAGAAGCTGAAAGAGGCCATCCGGGACACCA
ACAAGGCCGTGCAGTGCGTGCAAAGCAGCGTGGGCAATCTGATCTGCGCCATTAAGAG
CGTGCAGGACTACGTGAACAAAGAGATCGTCCCCTCTATCGCCAGACTGGGATGTGAA
GCTGCCGGACTGCAGCTGGGAATTGCCCTGACACAGCACTACAGCGAGCTGACCAACA
TCTTCGGCGACAACATCGGCAGCCTGCAAGAGAAGGGCATTAAGCTGCAGGCCATCGC
CAGCCTGTACCGCACCAACATCACCGAGATCTTCACCACCAGCACCGTGGATAAGTAC
GACATCTACGACCTGCTGTTCACCGAGAGCATCAAAGTGCGCGTGATCGACGTGGACC
TGAACGACTACAGCATCACCCTGCAAGTGCGGCTGCCTCTGCTGACCAGACTGCTGAA
CACCCAGATCTACAAGGTGGACAGCATCTCCTACAACATCCAGAACCGCGAGTGGTAC
ATCCCACTGCCTAGCCACATTATGACCAAGGGCGCCTTTCTCGGCGGAGCCGATGTGA
AAGAGTGCATCGAGGCCTTCAGCAGCTACATCTGCCCTAGCGATCCTGGCTTCGTGCT
GAACCACGAGATGGAAAGCTGCCTGAGCGGCAACATCTCTCAGTGCCCTAGAACCACC
GTGACCTCCGACATCGTGCCCAGATACGCCTTTGTGAATGGCGGCGTGGTGGCCAACT
GCATCACCACCACCTGTACCTGCAACGGCATCGGCAACCGGATCAACCAGCCTCCAGA
TCAGGGCGTGAAGATTATCACCCACAAAGAGTGTAACACCATCGGCATCAACGGCATG
CTGTTCAATACCAACAAAGAGGGCACCCTGGCCTTCTACACCCCTGACGATATCACCCT
GAACAACAGCGTGGCCCTGGATCCTATCGACATCTCCATCGAGCTGAACAAGGCCAAG
AGCGACCTGGAAGAAGCCAAAGAGTGGATCCGGCGGGCCAACCAGAAGCTGGATAGC
ATCGGAAGCTGGCACCAGAGCAGCACCACCATCATCGTGATCCTGATTATGATGATTAT
CCTGTTCATCATCAACATTACCATCATCACGATCGCCATCAAGTACTACCGGATCCAGAA
ACGGAACCGCGTGGACCAGAATGACAAGCCCTACGTGCTGACAAACAAG
>PIV3117_mRNA (SEQ ID NO: 415)
AUGCUGAUCUCCAUCCUGCUGAUCAUCACCACAAUGAUCAUGGCCAGCCACUGCCAG
AUCGACAUCACCAAGCUGCAGCACGUGGGCGUGCUGGUCAAUAGCCCUAAGGGCAU
GAAGAUCAGCCAGAACUUCGAGACACGCUACCUGAUCCUGUCUCUGAUCCCCAAGAU
CGAGGACAGCAACAGCUGCGGCGACCAGCAGAUCAAGCAGUACAAGCGGCUGCUGG
ACAGACUGAUCAUCCCUCUGUACGACGGCCUGCGGCUGCAGAAAGAUGUGAUCGUG
ACCAAUCAAGAGAGCAACGAGAACACAGACCCCAGAACCGAGAGAUUCUUCGGCGGC
GUGAUCGGCACAAUCGCCCUGGGAGUUGCUACAAGCGCCCAGAUUACAGCCGCCGU
GGCUCUGGUGGAAGCCAAGCAGGCCAGAAGCGACAUCGAGAAGCUGAAAGAGGCCA
UCCGGGACACCAACAAGGCCGUGCAGUGCGUGCAAAGCAGCGUGGGCAAUCUGAUC
UGCGCCAUUAAGAGCGUGCAGGACUACGUGAACAAAGAGAUCGUCCCCUCUAUCGCC
AGACUGGGAUGUGAAGCUGCCGGACUGCAGCUGGGAAUUGCCCUGACACAGCACUA
CAGCGAGCUGACCAACAUCUUCGGCGACAACAUCGGCAGCCUGCAAGAGAAGGGCAU
UAAGCUGCAGGCCAUCGCCAGCCUGUACCGCACCAACAUCACCGAGAUCUUCACCAC
CAGCACCGUGGAUAAGUACGACAUCUACGACCUGCUGUUCACCGAGAGCAUCAAAGU
GCGCGUGAUCGACGUGGACCUGAACGACUACAGCAUCACCCUGCAAGUGCGGCUGC
CUCUGCUGACCAGACUGCUGAACACCCAGAUCUACAAGGUGGACAGCAUCUCCUACA
ACAUCCAGAACCGCGAGUGGUACAUCCCACUGCCUAGCCACAUUAUGACCAAGGGCG
CCUUUCUCGGCGGAGCCGAUGUGAAAGAGUGCAUCGAGGCCUUCAGCAGCUACAUC
UGCCCUAGCGAUCCUGGCUUCGUGCUGAACCACGAGAUGGAAAGCUGCCUGAGCGG
CAACAUCUCUCAGUGCCCUAGAACCACCGUGACCUCCGACAUCGUGCCCAGAUACGC
CUUUGUGAAUGGCGGCGUGGUGGCCAACUGCAUCACCACCACCUGUACCUGCAACG
GCAUCGGCAACCGGAUCAACCAGCCUCCAGAUCAGGGCGUGAAGAUUAUCACCCACA
AAGAGUGUAACACCAUCGGCAUCAACGGCAUGCUGUUCAAUACCAACAAAGAGGGCA
CCCUGGCCUUCUACACCCCUGACGAUAUCACCCUGAACAACAGCGUGGCCCUGGAUC
CUAUCGACAUCUCCAUCGAGCUGAACAAGGCCAAGAGCGACCUGGAAGAAGCCAAAG
AGUGGAUCCGGCGGGCCAACCAGAAGCUGGAUAGCAUCGGAAGCUGGCACCAGAGC
AGCACCACCAUCAUCGUGAUCCUGAUUAUGAUGAUUAUCCUGUUCAUCAUCAACAUU
ACCAUCAUCACGAUCGCCAUCAAGUACUACCGGAUCCAGAAACGGAACCGCGUGGAC
CAGAAUGACAAGCCCUACGUGCUGACAAACAAG
>PIV3118_DNA (SEQ ID NO: 416)
ATGCTGATCTCCATCCTGCTGATCATCACCACAATGATCATGGCCAGCCACTGCCAGAT
CGACATCACCAAGCTGCAGCACGTGGGCGTGCTGGTCAATAGCCCTAAGGGCATGAAG
ATCAGCCAGAACTTCGAGACACGCTACCTGATCCTGTCTCTGATCCCCAAGATCGAGGA
CAGCAACAGCTGCGGCGACCAGCAGATCAAGCAGTACAAGCGGCTGCTGGACAGACT
GATCATCCCTCTGTACGACGGCCTGCGGCTGCAGAAAGATGTGATCGTGACCAATCAA
GAGAGCAACGAGAACACAGACCCCAGAACCGAGAGATTCTTCGGCGGCGTGATCGGC
ACAATCGCCCTGGGAGTTGCTACAAGCGCCCAGATTACAGCCGCCGTGGCTCTGGTGG
AAGCCAAGCAGGCCAGAAGCGACATCGAGAAGCTGAAAGAGGCCATCCGGGACACCA
ACAAGGCCGTGCAGTGCGTGCAAAGCAGCGTGGGCAATCTGATCTGCGCCATTAAGAG
CGTGCAGGACTACGTGAACAAAGAGATCGTCCCCTCTATCGCCAGACTGGGATGTGAA
GCTGCCGGACTGCAGCTGGGAATTGCCCTGACACAGCACTACAGCGAGCTGACCAACA
TCTTCGGCGACAACATCGGCAGCCTGCAAGAGAAGGGCATTAAGCTGCAGGCCATCGC
CAGCCTGTACCGCACCAACATCACCGAGATCTTCACCACCAGCACCGTGGATAAGTAC
GACATCTACGACCTGCTGTTCACCGAGAGCATCAAAGTGCGCGTGATCGACGTGGACC
TGAACGACTACAGCATCACCCTGCAAGTGCGGCTGCCTCTGCTGACCAGACTGCTGAA
CACCCAGATCTACAAGGTGGACAGCATCTCCTACAACATCCAGAACCGCGAGTGGTAC
ATCCCACTGCCTAGCCACATTATGACCAAGGGCGCCTTTCTCGGCGGAGCCGATGTGA
AAGAGTGCATCGAGGCCTTCAGCAGCTACATCTGCCCTAGCGATCCTGGCTTCGTGCT
GAACCACGAGATGGAAAGCTGCCTGAGCGGCAACATCTCTCAGTGCCCTAGAACCACC
GTGACCTCCGACATCGTGCCCAGATACGCCTTTGTGAATGGCGGCGTGGTGGCCAACT
GCATCACCACCACCTGTACCTGCAACGGCATCGGCAACCGGATCAACCAGCCTCCAGA
TCAGGGCGTGAAGATTATCACCCACAAAGAGTGTAACACCATCGGCATCAACGGCATG
CTGTTCAATACCAACAAAGAGGGCACCCTGGCCTTCTACACCCCTGACGATATCACCCT
GAACAACAGCGTGGCCCTGGATCCTATCGACATCTCCATCGAGCTGAACAAGCTCAAG
AGCGACCTGGAAGAAAGCAAAGAGTGGATCCGGCGGAGCAACCAGAAGCTGGATAGC
ATCGGAAGCTGGCACCAGAGCAGCACCACCATCATCGTGATCCTGATTATGATGATTAT
CCTGTTCATCATCAACATTACCATCATCACGATCGCCATCAAGTACTACCGGATCCAGAA
ACGGAACCGCGTGGACCAGAATGACAAGCCCTACGTGCTGACAAACAAG
>PIV3118_mRNA (SEQ ID NO: 417)
AUGCUGAUCUCCAUCCUGCUGAUCAUCACCACAAUGAUCAUGGCCAGCCACUGCCAG
AUCGACAUCACCAAGCUGCAGCACGUGGGCGUGCUGGUCAAUAGCCCUAAGGGCAU
GAAGAUCAGCCAGAACUUCGAGACACGCUACCUGAUCCUGUCUCUGAUCCCCAAGAU
CGAGGACAGCAACAGCUGCGGCGACCAGCAGAUCAAGCAGUACAAGCGGCUGCUGG
ACAGACUGAUCAUCCCUCUGUACGACGGCCUGCGGCUGCAGAAAGAUGUGAUCGUG
ACCAAUCAAGAGAGCAACGAGAACACAGACCCCAGAACCGAGAGAUUCUUCGGCGGC
GUGAUCGGCACAAUCGCCCUGGGAGUUGCUACAAGCGCCCAGAUUACAGCCGCCGU
GGCUCUGGUGGAAGCCAAGCAGGCCAGAAGCGACAUCGAGAAGCUGAAAGAGGCCA
UCCGGGACACCAACAAGGCCGUGCAGUGCGUGCAAAGCAGCGUGGGCAAUCUGAUC
UGCGCCAUUAAGAGCGUGCAGGACUACGUGAACAAAGAGAUCGUCCCCUCUAUCGCC
AGACUGGGAUGUGAAGCUGCCGGACUGCAGCUGGGAAUUGCCCUGACACAGCACUA
CAGCGAGCUGACCAACAUCUUCGGCGACAACAUCGGCAGCCUGCAAGAGAAGGGCAU
UAAGCUGCAGGCCAUCGCCAGCCUGUACCGCACCAACAUCACCGAGAUCUUCACCAC
CAGCACCGUGGAUAAGUACGACAUCUACGACCUGCUGUUCACCGAGAGCAUCAAAGU
GCGCGUGAUCGACGUGGACCUGAACGACUACAGCAUCACCCUGCAAGUGCGGCUGC
CUCUGCUGACCAGACUGCUGAACACCCAGAUCUACAAGGUGGACAGCAUCUCCUACA
ACAUCCAGAACCGCGAGUGGUACAUCCCACUGCCUAGCCACAUUAUGACCAAGGGCG
CCUUUCUCGGCGGAGCCGAUGUGAAAGAGUGCAUCGAGGCCUUCAGCAGCUACAUC
UGCCCUAGCGAUCCUGGCUUCGUGCUGAACCACGAGAUGGAAAGCUGCCUGAGCGG
CAACAUCUCUCAGUGCCCUAGAACCACCGUGACCUCCGACAUCGUGCCCAGAUACGC
CUUUGUGAAUGGCGGCGUGGUGGCCAACUGCAUCACCACCACCUGUACCUGCAACG
GCAUCGGCAACCGGAUCAACCAGCCUCCAGAUCAGGGCGUGAAGAUUAUCACCCACA
AAGAGUGUAACACCAUCGGCAUCAACGGCAUGCUGUUCAAUACCAACAAAGAGGGCA
CCCUGGCCUUCUACACCCCUGACGAUAUCACCCUGAACAACAGCGUGGCCCUGGAUC
CUAUCGACAUCUCCAUCGAGCUGAACAAGCUCAAGAGCGACCUGGAAGAAAGCAAAG
AGUGGAUCCGGCGGAGCAACCAGAAGCUGGAUAGCAUCGGAAGCUGGCACCAGAGC
AGCACCACCAUCAUCGUGAUCCUGAUUAUGAUGAUUAUCCUGUUCAUCAUCAACAUU
ACCAUCAUCACGAUCGCCAUCAAGUACUACCGGAUCCAGAAACGGAACCGCGUGGAC
CAGAAUGACAAGCCCUACGUGCUGACAAACAAG
>PIV3119_DNA (SEQ ID NO: 418)
ATGCTGATCTCCATCCTGCTGATCATCACCACAATGATCATGGCCAGCCACTGCCAGAT
CGACATCACCAAGCTGCAGCACGTGGGCGTGCTGGTCAATAGCCCTAAGGGCATGAAG
ATCAGCCAGAACTTCGAGACACGCTACCTGATCCTGTCTCTGATCCCCAAGATCGAGGA
CAGCAACAGCTGCGGCGACCAGCAGATCAAGCAGTACAAGCGGCTGCTGGACAGACT
GATCATCCCTCTGTACGACGGCCTGCGGCTGCAGAAAGATGTGATCGTGACCAATCAA
GAGAGCAACGAGAACACAGACCCCAGAACCGAGAGATTCTTCGGCGGCGTGATCGGC
ACAATCGCCCTGGGAGTTGCTACAAGCGCCCAGATTACAGCCGCCGTGGCTCTGGTGG
AAGCCAAGCAGGCCAGAAGCGACATCGAGAAGCTGAAAGAGGCCATCCGGGACACCA
ACAAGGCCGTGCAGTGCGTGCAAAGCAGCGTGGGCAATCTGATCTGCGCCATTAAGAG
CGTGCAGGACTACGTGAACAAAGAGATCGTCCCCTCTATCGCCAGACTGGGATGTGAA
GCTGCCGGACTGCAGCTGGGAATTGCCCTGACACAGCACTACAGCGAGCTGACCAACA
TCTTCGGCGACAACATCGGCAGCCTGCAAGAGAAGGGCATTAAGCTGCAGGCCATCGC
CAGCCTGTACCGCACCAACATCACCGAGATCTTCACCACCAGCACCGTGGATAAGTAC
GACATCTACGACCTGCTGTTCACCGAGAGCATCAAAGTGCGCGTGATCGACGTGGACC
TGAACGACTACAGCATCACCCTGCAAGTGCGGCTGCCTCTGCTGACCAGACTGCTGAA
CACCCAGATCTACAAGGTGGACAGCATCTCCTACAACATCCAGAACCGCGAGTGGTAC
ATCCCACTGCCTAGCCACATTATGACCAAGGGCGCCTTTCTCGGCGGAGCCGATGTGA
AAGAGTGCATCGAGGCCTTCAGCAGCTACATCTGCCCTAGCGATCCTGGCTTCGTGCT
GAACCACGAGATGGAAAGCTGCCTGAGCGGCAACATCTCTCAGTGCCCTAGAACCACC
GTGACCTCCGACATCGTGCCCAGATACGCCTTTGTGAATGGCGGCGTGGTGGCCAACT
GCATCACCACCACCTGTACCTGCAACGGCATCGGCAACCGGATCAACCAGCCTCCAGA
TCAGGGCGTGAAGATTATCACCCACAAAGAGTGTAACACCATCGGCATCAACGGCATG
CTGTTCAATACCAACAAAGAGGGCACCCTGGCCTTCTACACCCCTGACGATATCACCCT
GAACAACAGCGTGGCCCTGGATCCTATCGACATCTCCATCGAGCTGAACAAGCTCAAG
AGCGACCTGGAAGAAGCCAAAGAGTGGATCCGGGGGGCCAACCAGAAGCTGGATAGC
ATCGGAAGCTGGCACCAGAGCAGCACCACCATCATCGTGATCCTGATTATGATGATTAT
CCTGTTCATCATCAACATTACCATCATCACGATCGCCATCAAGTACTACCGGATCCAGAA
ACGGAACCGCGTGGACCAGAATGACAAGCCCTACGTGCTGACAAACAAG
>PIV3119_mRNA (SEQ ID NO: 419)
AUGCUGAUCUCCAUCCUGCUGAUCAUCACCACAAUGAUCAUGGCCAGCCACUGCCAG
AUCGACAUCACCAAGCUGCAGCACGUGGGCGUGCUGGUCAAUAGCCCUAAGGGCAU
GAAGAUCAGCCAGAACUUCGAGACACGCUACCUGAUCCUGUCUCUGAUCCCCAAGAU
CGAGGACAGCAACAGCUGCGGCGACCAGCAGAUCAAGCAGUACAAGCGGCUGCUGG
ACAGACUGAUCAUCCCUCUGUACGACGGCCUGCGGCUGCAGAAAGAUGUGAUCGUG
ACCAAUCAAGAGAGCAACGAGAACACAGACCCCAGAACCGAGAGAUUCUUCGGCGGC
GUGAUCGGCACAAUCGCCCUGGGAGUUGCUACAAGCGCCCAGAUUACAGCCGCCGU
GGCUCUGGUGGAAGCCAAGCAGGCCAGAAGCGACAUCGAGAAGCUGAAAGAGGCCA
UCCGGGACACCAACAAGGCCGUGCAGUGCGUGCAAAGCAGCGUGGGCAAUCUGAUC
UGCGCCAUUAAGAGCGUGCAGGACUACGUGAACAAAGAGAUCGUCCCCUCUAUCGCC
AGACUGGGAUGUGAAGCUGCCGGACUGCAGCUGGGAAUUGCCCUGACACAGCACUA
CAGCGAGCUGACCAACAUCUUCGGCGACAACAUCGGCAGCCUGCAAGAGAAGGGCAU
UAAGCUGCAGGCCAUCGCCAGCCUGUACCGCACCAACAUCACCGAGAUCUUCACCAC
CAGCACCGUGGAUAAGUACGACAUCUACGACCUGCUGUUCACCGAGAGCAUCAAAGU
GCGCGUGAUCGACGUGGACCUGAACGACUACAGCAUCACCCUGCAAGUGCGGCUGC
CUCUGCUGACCAGACUGCUGAACACCCAGAUCUACAAGGUGGACAGCAUCUCCUACA
ACAUCCAGAACCGCGAGUGGUACAUCCCACUGCCUAGCCACAUUAUGACCAAGGGCG
CCUUUCUCGGCGGAGCCGAUGUGAAAGAGUGCAUCGAGGCCUUCAGCAGCUACAUC
UGCCCUAGCGAUCCUGGCUUCGUGCUGAACCACGAGAUGGAAAGCUGCCUGAGCGG
CAACAUCUCUCAGUGCCCUAGAACCACCGUGACCUCCGACAUCGUGCCCAGAUACGC
CUUUGUGAAUGGCGGCGUGGUGGCCAACUGCAUCACCACCACCUGUACCUGCAACG
GCAUCGGCAACCGGAUCAACCAGCCUCCAGAUCAGGGCGUGAAGAUUAUCACCCACA
AAGAGUGUAACACCAUCGGCAUCAACGGCAUGCUGUUCAAUACCAACAAAGAGGGCA
CCCUGGCCUUCUACACCCCUGACGAUAUCACCCUGAACAACAGCGUGGCCCUGGAUC
CUAUCGACAUCUCCAUCGAGCUGAACAAGCUCAAGAGCGACCUGGAAGAAGCCAAAG
AGUGGAUCCGGCGGGCCAACCAGAAGCUGGAUAGCAUCGGAAGCUGGCACCAGAGC
AGCACCACCAUCAUCGUGAUCCUGAUUAUGAUGAUUAUCCUGUUCAUCAUCAACAUU
ACCAUCAUCACGAUCGCCAUCAAGUACUACCGGAUCCAGAAACGGAACCGCGUGGAC
CAGAAUGACAAGCCCUACGUGCUGACAAACAAG
SEQ ID NO: 420 - PIV3 precursor polypeptide (extended ectodomain with foldon and
tags)
MLISILLIITTMIMASHCQIDITKLQHVGVLVNSPKGMKISQNFETRYLILSLIPKIEDSNSCGDQ
QIKQYKRLLDRLIIPLYDGLRLQKDVIVTNQESNENTDPRTERFFGGVIGTIALGVATSAQITA
AVALVEAKQARSDIEKLKEAIRDTNKAVQSVQSSVGNLIVAIKSVQDYVNKEIVPSIARLGCEA
AGLQLGIALTQHYSELTNIFGDNIGSLQEKGIKLQGIASLYRTNITEIFTTSTVDKYDIYDLLFTE
SIKVRVIDVDLNDYSITLQVRLPLLTRLLNTQIYKVDSISYNIQNREWYIPLPSHIMTKGAFLGG
ADVKECIEAFSSYICPSDPGFVLNHEMESCLSGNISQCPRTTVTSDIVPRYAFVNGGVVANCI
TTTCTCNGIGNRINQPPDQGVKIITHKECNTIGINGMLFNTNKEGTLAFYTPDDITLNNSVALD
PIDISIELNKAKSDLEESKEWIRRSNQKLDSIGGGSGYIPEAPRDGQAYVRKDGEWVLLSTF
LGRSLEVLFQGPGSAWSHPQFEKAG
Annotated sequence of the PIV3 precursor polypeptide. Underlined sequence represents the
signal peptide, bolded sequence represents F2 polypeptide, and underlined and bolded
sequence represents the T4 fibritin foldon, PreScission cleavage site, Strep Tag Il and linker
sequences.
>PIV3031_DNA (SEQ ID NO: 294)
ATGCTGATCTCCATCCTGCTGATCATCACCACAATGATCATGGCCAGCCACTGCCAGAT
CGACATCACCAAGCTGCAGCACGTGGGCGTGCTGGTCAATAGCCCTAAGGGCATGAAG
ATCAGCCAGAACTTCGAGACACGCTACCTGATCCTGTCTCTGATCCCCAAGATCGAGGA
CAGCAACAGCTGCGGCGACCAGCAGATCAAGCAGTACAAGCGGCTGCTGGACAGACT
GATCATCCCTCTGTACGACGGCCTGCGGCTGCAGAAAGATGTGATCGTGACCAATCAA
GAGAGCAACGAGAACACAGACCCCAGAACCGAGAGATTCTTCGGCGGCGTGATCGGC
ACAATCGCCCTGGGAGTTGCTACAAGCGCCCAGATTACAGCCGCCGTGGCTCTGGTGG
AAGCCAAGCAGGCCAGAAGCGACATCGAGAAGCTGAAAGAGGCCATCCGGGACACCA
ACAAGGCCGTGCAGTGCGTGCAAAGCAGCGTGGGCAATCTGATCTGCGCCATTAAGAG
CGTGCAGGACTACGTGAACAAAGAGATCGTCCCCTCTATCGCCAGACTGGGATGTGAA
GCTGCCGGACTGCAGCTGGGAATTGCCCTGACACAGCACTACAGCGAGCTGACCAACA
TCTTCGGCGACAACATCGGCAGCCTGCAAGAGAAGGGCATTAAGCTGCAGGGAATCGC
CAGCCTGTACCGCACCAACATCACCGAGATCTTCACCACCAGCACCGTGGATAAGTAC
GACATCTACGACCTGCTGTTCACCGAGAGCATCAAAGTGCGCGTGATCGACGTGGACC
TGAACGACTACAGCATCACCCTGCAAGTGCGGCTGCCTCTGCTGACCAGACTGCTGAA
CACCCAGATCTACAAGGTGGACAGCATCTCCTACAACATCCAGAACCGCGAGTGGTAC
ATCCCACTGCCTAGCCACATTATGACCAAGGGCGCCTTTCTCGGCGGAGCCGATGTGA
AAGAGTGCATCGAGGCCTTCAGCAGCTACATCTGCCCTAGCGATCCTGGCTTCGTGCT
GAACCACGAGATGGAAAGCTGCCTGAGCGGCAACATCTCTCAGTGCCCTAGAACCACC
GTGACCTCCGACATCGTGCCCAGATACGCCTTTGTGAATGGCGGCGTGGTGGCCAACT
GCATCACCACCACCTGTACCTGCAACGGCATCGGCAACCGGATCAACCAGCCTCCAGA
TCAGGGCGTGAAGATTATCACCCACAAAGAGTGTAACACCATCGGCATCAACGGCATG
CTGTTCAATACCAACAAAGAGGGCACCCTGGCCTTCTACACCCCTGACGATATCACCCT
GAACAACAGCGTGGCCCTGGATCCTATCGACATCTCCATCGAGCTGAACAAGGCCAAG
AGCGACCTGGAAGAAAGCAAAGAGTGGATCCGGCGGAGCAACCAGAAGCTGGATAGC
ATCGGAAGCTGGCACCAGAGCAGCACCACCATCATCGTGATCCTGATTATGATGATTAT
CCTGTTCATCATCAACATTACCATCATCACGATCGCCATCAAGTACTACCGGATCCAGAA
ACGGAACCGCGTGGACCAGAATGACAAGCCCTACGTGCTGACAAACAAG
>PIV3031_mRNA (SEQ ID NO: 295)
AUGCUGAUCUCCAUCCUGCUGAUCAUCACCACAAUGAUCAUGGCCAGCCACUGCCAG
AUCGACAUCACCAAGCUGCAGCACGUGGGCGUGCUGGUCAAUAGCCCUAAGGGCAU
GAAGAUCAGCCAGAACUUCGAGACACGCUACCUGAUCCUGUCUCUGAUCCCCAAGAU
CGAGGACAGCAACAGCUGCGGCGACCAGCAGAUCAAGCAGUACAAGCGGCUGCUGG
ACAGACUGAUCAUCCCUCUGUACGACGGCCUGCGGCUGCAGAAAGAUGUGAUCGUG
ACCAAUCAAGAGAGCAACGAGAACACAGACCCCAGAACCGAGAGAUUCUUCGGCGGC
GUGAUCGGCACAAUCGCCCUGGGAGUUGCUACAAGCGCCCAGAUUACAGCCGCCGU
GGCUCUGGUGGAAGCCAAGCAGGCCAGAAGCGACAUCGAGAAGCUGAAAGAGGCCA
UCCGGGACACCAACAAGGCCGUGCAGUGCGUGCAAAGCAGCGUGGGCAAUCUGAUC
UGCGCCAUUAAGAGCGUGCAGGACUACGUGAACAAAGAGAUCGUCCCCUCUAUCGCC
AGACUGGGAUGUGAAGCUGCCGGACUGCAGCUGGGAAUUGCCCUGACACAGCACUA
CAGCGAGCUGACCAACAUCUUCGGCGACAACAUCGGCAGCCUGCAAGAGAAGGGCAU
UAAGCUGCAGGGAAUCGCCAGCCUGUACCGCACCAACAUCACCGAGAUCUUCACCAC
CAGCACCGUGGAUAAGUACGACAUCUACGACCUGCUGUUCACCGAGAGCAUCAAAGU
GCGCGUGAUCGACGUGGACCUGAACGACUACAGCAUCACCCUGCAAGUGCGGCUGC
CUCUGCUGACCAGACUGCUGAACACCCAGAUCUACAAGGUGGACAGCAUCUCCUACA
ACAUCCAGAACCGCGAGUGGUACAUCCCACUGCCUAGCCACAUUAUGACCAAGGGCG
CCUUUCUCGGCGGAGCCGAUGUGAAAGAGUGCAUCGAGGCCUUCAGCAGCUACAUC
UGCCCUAGCGAUCCUGGCUUCGUGCUGAACCACGAGAUGGAAAGCUGCCUGAGCGG
CAACAUCUCUCAGUGCCCUAGAACCACCGUGACCUCCGACAUCGUGCCCAGAUACGC
CUUUGUGAAUGGCGGCGUGGUGGCCAACUGCAUCACCACCACCUGUACCUGCAACG
GCAUCGGCAACCGGAUCAACCAGCCUCCAGAUCAGGGCGUGAAGAUUAUCACCCACA
AAGAGUGUAACACCAUCGGCAUCAACGGCAUGCUGUUCAAUACCAACAAAGAGGGCA
CCCUGGCCUUCUACACCCCUGACGAUAUCACCCUGAACAACAGCGUGGCCCUGGAUC
CUAUCGACAUCUCCAUCGAGCUGAACAAGGCCAAGAGCGACCUGGAAGAAAGCAAAG
AGUGGAUCCGGCGGAGCAACCAGAAGCUGGAUAGCAUCGGAAGCUGGCACCAGAGC
AGCACCACCAUCAUCGUGAUCCUGAUUAUGAUGAUUAUCCUGUUCAUCAUCAACAUU
ACCAUCAUCACGAUCGCCAUCAAGUACUACCGGAUCCAGAAACGGAACCGCGUGGAC
CAGAAUGACAAGCCCUACGUGCUGACAAACAAG
>PIV3135_DNA (SEQ ID NO: 296)
ATGCTGATCTCCATCCTGCTGATCATCACCACAATGATCATGGCCAGCCACTGCCAGAT
CGACATCACCAAGCTGCAGCACGTGGGCGTGCTGGTCAATAGCCCTAAGGGCATGAAG
ATCAGCCAGAACTTCGAGACACGCTACCTGATCCTGTCTCTGATCCCCAAGATCGAGGA
CAGCAACAGCTGCGGCGACCAGCAGATCAAGCAGTACAAGCGGCTGCTGGACAGACT
GATCATCCCTCTGTACGACGGCCTGCGGCTGCAGAAAGATGTGATCGTGACCAATCAA
GAGAGCAACGAGAACACAGACCCCAGAACCGAGAGATTCTTCGGCGGCGTGATCGGC
ACAATCGCCCTGGGAGTTGCTACAAGCGCCCAGATTACAGCCGCCGTGGCTCTGGTGG
AAGCCAAGCAGGCCAGAAGCGACATCGAGAAGCTGAAAGAGGCCATCCGGGACACCA
ACAAGGCCGTGCAGTCTGTGCAAAGCAGCGTGGGCAATCTGATCGTGGCCATTAAGAG
CGTGCAGGACTACGTGAACAAAGAGATCGTCCCCTCTATCGCCAGACTGGGATGTGAA
GCTGCCGGACTGCAGCTGGGAATTGCCCTGACACAGCACTACAGCTGCCTGACCAACA
TCTTCGGCGACAACATCGGCAGCCTGCAAGAGAAGGGCATTAAGCTGCAGGGAATCGC
CAGCTGCTACCGCACCAACATCACCGAGATCTTCACCACCAGCACCGTGGATAAGTAC
GACATCTACGACCTGCTGTTCACCGAGAGCATCAAAGTGCGCGTGATCGACGTGGACC
TGAACGACTACAGCATCACCCTGCAAGTGCGGCTGCCTCTGCTGACCAGACTGCTGAA
CACCCAGATCTACAAGGTGGACAGCATCTCCTACAACATCCAGAACCGCGAGTGGTAC
ATCCCACTGCCTAGCCACATTATGACCAAGGGCGCCTTTCTCGGCGGAGCCGATGTGA
AAGAGTGCATCGAGGCCTTCAGCAGCTACATCTGCCCTAGCGATCCTGGCTTCGTGCT
GAACCACGAGATGGAAAGCTGCCTGAGCGGCAACATCTCTCAGTGCCCTAGAACCACC
GTGACCTCCGACATCGTGCCCAGATACGCCTTTGTGAATGGCGGCGTGGTGGCCAACT
GCATCACCACCACCTGTACCTGCAACGGCATCGGCAACCGGATCAACCAGCCTCCAGA
TCAGGGCGTGAAGATTATCACCCACAAAGAGTGTAACACCATCGGCATCAACGGCATG
CTGTTCAATACCAACAAAGAGGGCACCCTGGCCTTCTACACCCCTGACGATATCACCCT
GAACAACAGCGTGGCCCTGGATCCTATCGACATCTCCATCGAGCTGAACAAGGCCAAG
AGCGACCTGGAAGAAAGCAAAGAGTGGATCCGGCGGAGCAACCAGAAGCTGGATAGC
ATCGGAAGCTGGCACCAGAGCAGCACCACCATCATCGTGATCCTGATTATGATGATTAT
CCTGTTCATCATCAACATTACCATCATCACGATCGCCATCAAGTACTACCGGATCCAGAA
ACGGAACCGCGTGGACCAGAATGACAAGCCCTACGTGCTGACAAACAAG
>PIV3135_mRNA (SEQ ID NO: 297)
AUGCUGAUCUCCAUCCUGCUGAUCAUCACCACAAUGAUCAUGGCCAGCCACUGCCAG
AUCGACAUCACCAAGCUGCAGCACGUGGGCGUGCUGGUCAAUAGCCCUAAGGGCAU
GAAGAUCAGCCAGAACUUCGAGACACGCUACCUGAUCCUGUCUCUGAUCCCCAAGAU
CGAGGACAGCAACAGCUGCGGCGACCAGCAGAUCAAGCAGUACAAGCGGCUGCUGG
ACAGACUGAUCAUCCCUCUGUACGACGGCCUGCGGCUGCAGAAAGAUGUGAUCGUG
ACCAAUCAAGAGAGCAACGAGAACACAGACCCCAGAACCGAGAGAUUCUUCGGCGGC
GUGAUCGGCACAAUCGCCCUGGGAGUUGCUACAAGCGCCCAGAUUACAGCCGCCGU
GGCUCUGGUGGAAGCCAAGCAGGCCAGAAGCGACAUCGAGAAGCUGAAAGAGGCCA
UCCGGGACACCAACAAGGCCGUGCAGUCUGUGCAAAGCAGCGUGGGCAAUCUGAUC
GUGGCCAUUAAGAGCGUGCAGGACUACGUGAACAAAGAGAUCGUCCCCUCUAUCGC
CAGACUGGGAUGUGAAGCUGCCGGACUGCAGCUGGGAAUUGCCCUGACACAGCACU
ACAGCUGCCUGACCAACAUCUUCGGCGACAACAUCGGCAGCCUGCAAGAGAAGGGCA
UUAAGCUGCAGGGAAUCGCCAGCUGCUACCGCACCAACAUCACCGAGAUCUUCACCA
CCAGCACCGUGGAUAAGUACGACAUCUACGACCUGCUGUUCACCGAGAGCAUCAAAG
UGCGCGUGAUCGACGUGGACCUGAACGACUACAGCAUCACCCUGCAAGUGCGGCUG
CCUCUGCUGACCAGACUGCUGAACACCCAGAUCUACAAGGUGGACAGCAUCUCCUAC
AACAUCCAGAACCGCGAGUGGUACAUCCCACUGCCUAGCCACAUUAUGACCAAGGGC
GCCUUUCUCGGCGGAGCCGAUGUGAAAGAGUGCAUCGAGGCCUUCAGCAGCUACAU
CUGCCCUAGCGAUCCUGGCUUCGUGCUGAACCACGAGAUGGAAAGCUGCCUGAGCG
GCAACAUCUCUCAGUGCCCUAGAACCACCGUGACCUCCGACAUCGUGCCCAGAUACG
CCUUUGUGAAUGGCGGCGUGGUGGCCAACUGCAUCACCACCACCUGUACCUGCAAC
GGCAUCGGCAACCGGAUCAACCAGCCUCCAGAUCAGGGCGUGAAGAUUAUCACCCAC
AAAGAGUGUAACACCAUCGGCAUCAACGGCAUGCUGUUCAAUACCAACAAAGAGGGC
ACCCUGGCCUUCUACACCCCUGACGAUAUCACCCUGAACAACAGCGUGGCCCUGGAU
CCUAUCGACAUCUCCAUCGAGCUGAACAAGGCCAAGAGCGACCUGGAAGAAAGCAAA
GAGUGGAUCCGGCGGAGCAACCAGAAGCUGGAUAGCAUCGGAAGCUGGCACCAGAG
CAGCACCACCAUCAUCGUGAUCCUGAUUAUGAUGAUUAUCCUGUUCAUCAUCAACAU
UACCAUCAUCACGAUCGCCAUCAAGUACUACCGGAUCCAGAAACGGAACCGCGUGGA
CCAGAAUGACAAGCCCUACGUGCUGACAAACAAG
>PIV3138_DNA (SEQ ID NO: 298)
ATGCTGATCTCCATCCTGCTGATCATCACCACAATGATCATGGCCAGCCACTGCCAGAT
CGACATCACCAAGCTGCAGCACGTGGGCGTGCTGGTCAATAGCCCTAAGGGCATGAAG
ATCAGCCAGAACTTCGAGACACGCTACCTGATCCTGTCTCTGATCCCCAAGATCGAGGA
CAGCAACAGCTGCGGCGACCAGCAGATCAAGCAGTACAAGCGGCTGCTGGACAGACT
GATCATCCCTCTGTACGACGGCCTGCGGCTGCAGAAAGATGTGATCGTGACCAATCAA
GAGAGCAACGAGAACACAGACCCCAGAACCGAGAGATTCTTCGGCGGCGTGATCGGC
ACAATCGCCCTGGGAGTTGCTACAAGCGCCCAGATTACAGCCGCCGTGGCTCTGGTGG
AAGCCAAGCAGGCCAGAAGCGACATCGAGAAGCTGAAAGAGGCCATCCGGGACACCA
ACAAGGCCGTGCAGTCTGTGCAAAGCAGCGTGGGCAATCTGATCGTGGCCATTAAGAG
CGTGCAGGACTACGTGAACAAAGAGATCGTCCCCTCTATCGCCAGACTGGGATGTGAA
GCTGCCGGACTGCAGCTGGGAATTGCCCTGACACAGCACTACAGCGAGCTGACCAACA
TCTTCGGCGACAACATCGGCAGCCTGCAAGAGAAGGGCATTAAGCTGCAGGGAATCGC
CAGCCTGTACCGCACCAACATCACCGAGATCTTCACCACCAGCACCGTGGATAAGTAC
GACATCTACGACCTGCTGTTCACCGAGAGCATCAAAGTGCGCGTGATCGACGTGGACC
TGAACGACTACAGCATCACCCTGCAAGTGCGGCTGCCTCTGCTGACCAGACTGCTGAA
CACCCAGATCTACAAGGTGGACAGCATCTCCTACAACATCCAGAACCGCGAGTGGTAC
ATCCCACTGCCTAGCCACATTATGACCAAGGGCGCCTTTCTCGGCGGAGCCGATGTGA
AAGAGTGCATCGAGGCCTTCAGCAGCTACATCTGCCCTAGCGATCCTGGCTTCGTGCT
GAACCACGAGATGGAAAGCTGCCTGAGCGGCAACATCTCTCAGTGCCCTAGAACCACC
GTGACCTCCGACATCGTGCCCAGATACGCCTTTGTGAATGGCGGCGTGGTGGCCAACT
GCATCACCACCACCTGTACCTGCAACGGCATCGGCAACCGGATCAACCAGCCTCCAGA
TCAGGGCGTGAAGATTATCACCCACAAAGAGTGTAACACCATCGGCATCAACGGCATG
CTGTTCAATACCAACAAAGAGGGCACCCTGGCCTTCTACACCCCTGACGATATCACCCT
GAACAACAGCGTGGCCCTGGATCCTATCGACATCTCCATCGAGCTGAACAAGCTCAAG
AGCGACCTGGAAGAACTGAAAGAGTGGATCCGGCGGAGCAACCAGAAGCTGGATAGC
ATCGGAAGCTGGCACCAGAGCAGCACCACCATCATCGTGATCCTGATTATGATGATTAT
CCTGTTCATCATCAACATTACCATCATCACGATCGCCATCAAGTACTACCGGATCCAGAA
ACGGAACCGCGTGGACCAGAATGACAAGCCCTACGTGCTGACAAACAAG
>PIV3138_mRNA (SEQ ID NO: 299)
AUGCUGAUCUCCAUCCUGCUGAUCAUCACCACAAUGAUCAUGGCCAGCCACUGCCAG
AUCGACAUCACCAAGCUGCAGCACGUGGGCGUGCUGGUCAAUAGCCCUAAGGGCAU
GAAGAUCAGCCAGAACUUCGAGACACGCUACCUGAUCCUGUCUCUGAUCCCCAAGAU
CGAGGACAGCAACAGCUGCGGCGACCAGCAGAUCAAGCAGUACAAGCGGCUGCUGG
ACAGACUGAUCAUCCCUCUGUACGACGGCCUGCGGCUGCAGAAAGAUGUGAUCGUG
ACCAAUCAAGAGAGCAACGAGAACACAGACCCCAGAACCGAGAGAUUCUUCGGCGGC
GUGAUCGGCACAAUCGCCCUGGGAGUUGCUACAAGCGCCCAGAUUACAGCCGCCGU
GGCUCUGGUGGAAGCCAAGCAGGCCAGAAGCGACAUCGAGAAGCUGAAAGAGGCCA
UCCGGGACACCAACAAGGCCGUGCAGUCUGUGCAAAGCAGCGUGGGCAAUCUGAUC
GUGGCCAUUAAGAGCGUGCAGGACUACGUGAACAAAGAGAUCGUCCCCUCUAUCGC
CAGACUGGGAUGUGAAGCUGCCGGACUGCAGCUGGGAAUUGCCCUGACACAGCACU
ACAGCGAGCUGACCAACAUCUUCGGCGACAACAUCGGCAGCCUGCAAGAGAAGGGCA
UUAAGCUGCAGGGAAUCGCCAGCCUGUACCGCACCAACAUCACCGAGAUCUUCACCA
CCAGCACCGUGGAUAAGUACGACAUCUACGACCUGCUGUUCACCGAGAGCAUCAAAG
UGCGCGUGAUCGACGUGGACCUGAACGACUACAGCAUCACCCUGCAAGUGCGGCUG
CCUCUGCUGACCAGACUGCUGAACACCCAGAUCUACAAGGUGGACAGCAUCUCCUAC
AACAUCCAGAACCGCGAGUGGUACAUCCCACUGCCUAGCCACAUUAUGACCAAGGGC
GCCUUUCUCGGCGGAGCCGAUGUGAAAGAGUGCAUCGAGGCCUUCAGCAGCUACAU
CUGCCCUAGCGAUCCUGGCUUCGUGCUGAACCACGAGAUGGAAAGCUGCCUGAGCG
GCAACAUCUCUCAGUGCCCUAGAACCACCGUGACCUCCGACAUCGUGCCCAGAUACG
CCUUUGUGAAUGGCGGCGUGGUGGCCAACUGCAUCACCACCACCUGUACCUGCAAC
GGCAUCGGCAACCGGAUCAACCAGCCUCCAGAUCAGGGCGUGAAGAUUAUCACCCAC
AAAGAGUGUAACACCAUCGGCAUCAACGGCAUGCUGUUCAAUACCAACAAAGAGGGC
ACCCUGGCCUUCUACACCCCUGACGAUAUCACCCUGAACAACAGCGUGGCCCUGGAU
CCUAUCGACAUCUCCAUCGAGCUGAACAAGCUCAAGAGCGACCUGGAAGAACUGAAA
GAGUGGAUCCGGCGGAGCAACCAGAAGCUGGAUAGCAUCGGAAGCUGGCACCAGAG
CAGCACCACCAUCAUCGUGAUCCUGAUUAUGAUGAUUAUCCUGUUCAUCAUCAACAU
UACCAUCAUCACGAUCGCCAUCAAGUACUACCGGAUCCAGAAACGGAACCGCGUGGA
CCAGAAUGACAAGCCCUACGUGCUGACAAACAAG
>PIV3140_DNA (SEQ ID NO: 503)
ATGCTGATCTCCATCCTGCTGATCATCACCACAATGATCATGGCCAGCCACTGCCAGAT
CGACATCACCAAGCTGCAGCACGTGGGCGTGCTGGTCAATAGCCCTAAGGGCATGAAG
ATCAGCCAGAACTTCGAGACACGCTACCTGATCCTGTCTCTGATCCCCAAGATCGAGGA
CAGCAACAGCTGCGGCGACCAGCAGATCAAGCAGTACAAGCGGCTGCTGGACAGACT
GATCATCCCTCTGTACGACGGCCTGCGGCTGCAGAAAGATGTGATCGTGACCAATCAA
GAGAGCAACGAGAACACAGACCCCAGAACCGAGAGATTCTTCGGCGGCGTGATCGGC
ACAATCGCCCTGGGAGTTGCTACAAGCGCCCAGATTACAGCCGCCGTGGCTCTGGTGG
AAGCCAAGCAGGCCAGAAGCGACATCGAGAAGCTGAAAGAGGCCATCCGGGACACCA
ACAAGGCCGTGCAGTGCGTGCAAAGCAGCGTGGGCAATCTGATCTGCGCCATTAAGAG
CGTGCAGGACTACGTGAACAAAGAGATCGTCCCCTCTATCGCCAGACTGGGATGTGAA
GCTGCCGGACTGCAGCTGGGAATTGCCCTGACACAGCACTACAGCTGCCTGACCAACA
TCTTCGGCGACAACATCGGCAGCCTGCAAGAGAAGGGCATTAAGCTGCAGGGAATCGC
CAGCTGCTACCGCACCAACATCACCGAGATCTTCACCACCAGCACCGTGGATAAGTAC
GACATCTACGACCTGCTGTTCACCGAGAGCATCAAAGTGCGCGTGATCGACGTGGACC
TGAACGACTACAGCATCACCCTGCAAGTGCGGCTGCCTCTGCTGACCAGACTGCTGAA
CACCCAGATCTACAAGGTGGACAGCATCTCCTACAACATCCAGAACCGCGAGTGGTAC
ATCCCACTGCCTAGCCACATTATGACCAAGGGCGCCTTTCTCGGCGGAGCCGATGTGA
AAGAGTGCATCGAGGCCTTCAGCAGCTACATCTGCCCTAGCGATCCTGGCTTCGTGCT
GAACCACGAGATGGAAAGCTGCCTGAGCGGCAACATCTCTCAGTGCCCTAGAACCACC
GTGACCTCCGACATCGTGCCCAGATACGCCTTTGTGAATGGCGGCGTGGTGGCCAACT
GCATCACCACCACCTGTACCTGCAACGGCATCGGCAACCGGATCAACCAGCCTCCAGA
TCAGGGCGTGAAGATTATCACCCACAAAGAGTGTAACACCATCGGCATCAACGGCATG
CTGTTCAATACCAACAAAGAGGGCACCCTGGCCTTCTACACCCCTGACGATATCACCCT
GAACAACAGCGTGGCCCTGGATCCTATCGACATCTCCATCGAGCTGAACAAGCTCAAG
AGCGACCTGGAAGAACTGAAAGAGTGGATCCGGCGGAGCAACCAGAAGCTGGATAGC
ATCGGAAGCTGGCACCAGAGCAGCACCACCATCATCGTGATCCTGATTATGATGATTAT
CCTGTTCATCATCAACATTACCATCATCACGATCGCCATCAAGTACTACCGGATCCAGAA
ACGGAACCGCGTGGACCAGAATGACAAGCCCTACGTGCTGACAAACAAG
>PIV3140_mRNA (SEQ ID NO: 504)
AUGCUGAUCUCCAUCCUGCUGAUCAUCACCACAAUGAUCAUGGCCAGCCACUGCCAG
AUCGACAUCACCAAGCUGCAGCACGUGGGCGUGCUGGUCAAUAGCCCUAAGGGCAU
GAAGAUCAGCCAGAACUUCGAGACACGCUACCUGAUCCUGUCUCUGAUCCCCAAGAU
CGAGGACAGCAACAGCUGCGGCGACCAGCAGAUCAAGCAGUACAAGCGGCUGCUGG
ACAGACUGAUCAUCCCUCUGUACGACGGCCUGCGGCUGCAGAAAGAUGUGAUCGUG
ACCAAUCAAGAGAGCAACGAGAACACAGACCCCAGAACCGAGAGAUUCUUCGGCGGC
GUGAUCGGCACAAUCGCCCUGGGAGUUGCUACAAGCGCCCAGAUUACAGCCGCCGU
GGCUCUGGUGGAAGCCAAGCAGGCCAGAAGCGACAUCGAGAAGCUGAAAGAGGCCA
UCCGGGACACCAACAAGGCCGUGCAGUGCGUGCAAAGCAGCGUGGGCAAUCUGAUC
UGCGCCAUUAAGAGCGUGCAGGACUACGUGAACAAAGAGAUCGUCCCCUCUAUCGCC
AGACUGGGAUGUGAAGCUGCCGGACUGCAGCUGGGAAUUGCCCUGACACAGCACUA
CAGCUGCCUGACCAACAUCUUCGGCGACAACAUCGGCAGCCUGCAAGAGAAGGGCAU
UAAGCUGCAGGGAAUCGCCAGCUGCUACCGCACCAACAUCACCGAGAUCUUCACCAC
CAGCACCGUGGAUAAGUACGACAUCUACGACCUGCUGUUCACCGAGAGCAUCAAAGU
GCGCGUGAUCGACGUGGACCUGAACGACUACAGCAUCACCCUGCAAGUGCGGCUGC
CUCUGCUGACCAGACUGCUGAACACCCAGAUCUACAAGGUGGACAGCAUCUCCUACA
ACAUCCAGAACCGCGAGUGGUACAUCCCACUGCCUAGCCACAUUAUGACCAAGGGCG
CCUUUCUCGGCGGAGCCGAUGUGAAAGAGUGCAUCGAGGCCUUCAGCAGCUACAUC
UGCCCUAGCGAUCCUGGCUUCGUGCUGAACCACGAGAUGGAAAGCUGCCUGAGCGG
CAACAUCUCUCAGUGCCCUAGAACCACCGUGACCUCCGACAUCGUGCCCAGAUACGC
CUUUGUGAAUGGCGGCGUGGUGGCCAACUGCAUCACCACCACCUGUACCUGCAACG
GCAUCGGCAACCGGAUCAACCAGCCUCCAGAUCAGGGCGUGAAGAUUAUCACCCACA
AAGAGUGUAACACCAUCGGCAUCAACGGCAUGCUGUUCAAUACCAACAAAGAGGGCA
CCCUGGCCUUCUACACCCCUGACGAUAUCACCCUGAACAACAGCGUGGCCCUGGAUC
CUAUCGACAUCUCCAUCGAGCUGAACAAGCUCAAGAGCGACCUGGAAGAACUGAAAG
AGUGGAUCCGGCGGAGCAACCAGAAGCUGGAUAGCAUCGGAAGCUGGCACCAGAGC
AGCACCACCAUCAUCGUGAUCCUGAUUAUGAUGAUUAUCCUGUUCAUCAUCAACAUU
ACCAUCAUCACGAUCGCCAUCAAGUACUACCGGAUCCAGAAACGGAACCGCGUGGAC
CAGAAUGACAAGCCCUACGUGCUGACAAACAAG
>PIV3141_DNA (SEQ ID NO: 505)
ATGCTGATCTCCATCCTGCTGATCATCACCACAATGATCATGGCCAGCCACTGCCAGAT
CGACATCACCAAGCTGCAGCACGTGGGCGTGCTGGTCAATAGCCCTAAGGGCATGAAG
ATCAGCCAGAACTTCGAGACACGCTACCTGATCCTGTCTCTGATCCCCAAGATCGAGGA
CAGCAACAGCTGCGGCGACCAGCAGATCAAGCAGTACAAGCGGCTGCTGGACAGACT
GATCATCCCTCTGTACGACGGCCTGCGGCTGCAGAAAGATGTGATCGTGACCAATCAA
GAGAGCAACGAGAACACAGACCCCAGAACCGAGAGATTCTTCGGCGGCGTGATCGGC
ACAATCGCCCTGGGAGTTGCTACAAGCGCCCAGATTACAGCCGCCGTGGCTCTGGTGG
AAGCCAAGCAGGCCAGAAGCGACATCGAGAAGCTGAAAGAGGCCATCCGGGACACCA
ACAAGGCCGTGCAGTGCGTGCAAAGCAGCGTGGGCAATCTGATCTGCGCCATTAAGAG
CGTGCAGGACTACGTGAACAAAGAGATCGTCCCCTCTATCGCCAGACTGGGATGTGAA
GCTGCCGGACTGCAGCTGGGAATTGCCCTGACACAGCACTACAGCTGCCTGACCAACA
TCTTCGGCGACAACATCGGCAGCCTGCAAGAGAAGGGCATTAAGCTGCAGGGAATCGC
CAGCTGCTACCGCACCAACATCACCGAGATCTTCACCACCAGCACCGTGGATAAGTAC
GACATCTACGACCTGCTGTTCACCGAGAGCATCAAAGTGCGCGTGATCGACGTGGACC
TGAACGACTACAGCATCACCCTGCAAGTGCGGCTGCCTCTGCTGACCAGACTGCTGAA
CACCCAGATCTACAAGGTGGACAGCATCTCCTACAACATCCAGAACCGCGAGTGGTAC
ATCCCACTGCCTAGCCACATTATGACCAAGGGCGCCTTTCTCGGCGGAGCCGATGTGA
AAGAGTGCATCGAGGCCTTCAGCAGCTACATCTGCCCTAGCGATCCTGGCTTCGTGCT
GAACCACGAGATGGAAAGCTGCCTGAGCGGCAACATCTCTCAGTGCCCTAGAACCACC
GTGACCTCCGACATCGTGCCCAGATACGCCTTTGTGAATGGCGGCGTGGTGGCCAACT
GCATCACCACCACCTGTACCTGCAACGGCATCGGCAACCGGATCAACCAGCCTCCAGA
TCAGGGCGTGAAGATTATCACCCACAAAGAGTGTAACACCATCGGCATCAACGGCATG
CTGTTCAATACCAACAAAGAGGGCACCCTGGCCTTCTACACCCCTGACGATATCACCCT
GAACAACAGCGTGGCCCTGGATCCTATCGACATCTCCATCGAGCTGAACAAGCTCAAG
AGCGACCTGGAAGAAAGCAAAGAGTGGTTCCGGCGGAGCAACCAGAAGCTGGATAGC
ATCGGAAGCTGGCACCAGAGCAGCACCACCATCATCGTGATCCTGATTATGATGATTAT
CCTGTTCATCATCAACATTACCATCATCACGATCGCCATCAAGTACTACCGGATCCAGAA
ACGGAACCGCGTGGACCAGAATGACAAGCCCTACGTGCTGACAAACAAG
>PIV3141_mRNA (SEQ ID NO: 506)
AUGCUGAUCUCCAUCCUGCUGAUCAUCACCACAAUGAUCAUGGCCAGCCACUGCCAG
AUCGACAUCACCAAGCUGCAGCACGUGGGCGUGCUGGUCAAUAGCCCUAAGGGCAU
GAAGAUCAGCCAGAACUUCGAGACACGCUACCUGAUCCUGUCUCUGAUCCCCAAGAU
CGAGGACAGCAACAGCUGCGGCGACCAGCAGAUCAAGCAGUACAAGCGGCUGCUGG
ACAGACUGAUCAUCCCUCUGUACGACGGCCUGCGGCUGCAGAAAGAUGUGAUCGUG
ACCAAUCAAGAGAGCAACGAGAACACAGACCCCAGAACCGAGAGAUUCUUCGGCGGC
GUGAUCGGCACAAUCGCCCUGGGAGUUGCUACAAGCGCCCAGAUUACAGCCGCCGU
GGCUCUGGUGGAAGCCAAGCAGGCCAGAAGCGACAUCGAGAAGCUGAAAGAGGCCA
UCCGGGACACCAACAAGGCCGUGCAGUGCGUGCAAAGCAGCGUGGGCAAUCUGAUC
UGCGCCAUUAAGAGCGUGCAGGACUACGUGAACAAAGAGAUCGUCCCCUCUAUCGCC
AGACUGGGAUGUGAAGCUGCCGGACUGCAGCUGGGAAUUGCCCUGACACAGCACUA
CAGCUGCCUGACCAACAUCUUCGGCGACAACAUCGGCAGCCUGCAAGAGAAGGGCAU
UAAGCUGCAGGGAAUCGCCAGCUGCUACCGCACCAACAUCACCGAGAUCUUCACCAC
CAGCACCGUGGAUAAGUACGACAUCUACGACCUGCUGUUCACCGAGAGCAUCAAAGU
GCGCGUGAUCGACGUGGACCUGAACGACUACAGCAUCACCCUGCAAGUGCGGCUGC
CUCUGCUGACCAGACUGCUGAACACCCAGAUCUACAAGGUGGACAGCAUCUCCUACA
ACAUCCAGAACCGCGAGUGGUACAUCCCACUGCCUAGCCACAUUAUGACCAAGGGCG
CCUUUCUCGGCGGAGCCGAUGUGAAAGAGUGCAUCGAGGCCUUCAGCAGCUACAUC
UGCCCUAGCGAUCCUGGCUUCGUGCUGAACCACGAGAUGGAAAGCUGCCUGAGCGG
CAACAUCUCUCAGUGCCCUAGAACCACCGUGACCUCCGACAUCGUGCCCAGAUACGC
CUUUGUGAAUGGCGGCGUGGUGGCCAACUGCAUCACCACCACCUGUACCUGCAACG
GCAUCGGCAACCGGAUCAACCAGCCUCCAGAUCAGGGCGUGAAGAUUAUCACCCACA
AAGAGUGUAACACCAUCGGCAUCAACGGCAUGCUGUUCAAUACCAACAAAGAGGGCA
CCCUGGCCUUCUACACCCCUGACGAUAUCACCCUGAACAACAGCGUGGCCCUGGAUC
CUAUCGACAUCUCCAUCGAGCUGAACAAGCUCAAGAGCGACCUGGAAGAAAGCAAAG
AGUGGUUCCGGCGGAGCAACCAGAAGCUGGAUAGCAUCGGAAGCUGGCACCAGAGC
AGCACCACCAUCAUCGUGAUCCUGAUUAUGAUGAUUAUCCUGUUCAUCAUCAACAUU
ACCAUCAUCACGAUCGCCAUCAAGUACUACCGGAUCCAGAAACGGAACCGCGUGGAC
CAGAAUGACAAGCCCUACGUGCUGACAAACAAG
>PIV3165_DNA (SEQ ID NO: 507)
ATGCTGATCTCCATCCTGCTGATCATCACCACAATGATCATGGCCAGCCACTGCCAGAT
CGACATCACCAAGCTGCAGCACGTGGGCGTGCTGGTCAATAGCCCTAAGGGCATGAAG
ATCAGCCAGAACTTCGAGACACGCTACCTGATCCTGTCTCTGATCCCCAAGATCGAGGA
CAGCAACAGCTGCGGCGACCAGCAGATCAAGCAGTACAAGCGGCTGCTGGACAGACT
GATCATCCCTCTGTACGACGGCCTGCGGCTGCAGAAAGATGTGATCGTGACCAATCAA
GAGAGCAACGAGAACACAGACCCCAGAACCGAGAGAGGCAGCGGCGGCGTGATCGGC
ACAATCGCCCTGGGAGTTGCTACAAGCGCCCAGATTACAGCCGCCGTGGCTCTGGTGG
AAGCCAAGCAGGCCAGAAGCGACATCGAGAAGCTGAAAGAGGCCATCCGGGACACCA
ACAAGGCCGTGCAGTGCGTGCAAAGCAGCGTGGGCAATCTGATCTGCGCCATTAAGAG
CGTGCAGGACTACGTGAACAAAGAGATCGTCCCCTCTATCGCCAGACTGGGATGTGAA
GCTGCCGGACTGCAGCTGGGAATTGCCCTGACACAGCACTACAGCTGCCTGACCAACA
TCTTCGGCGACAACATCGGCAGCCTGCAAGAGAAGGGCATTAAGCTGCAGGGAATCGC
CAGCTGCTACCGCACCAACATCACCGAGATCTTCACCACCAGCACCGTGGATAAGTAC
GACATCTACGACCTGCTGTTCACCGAGAGCATCAAAGTGCGCGTGATCGACGTGGACC
TGAACGACTACAGCATCACCCTGCAAGTGCGGCTGCCTCTGCTGACCAGACTGCTGAA
CACCCAGATCTACAAGGTGGACAGCATCTCCTACAACATCCAGAACCGCGAGTGGTAC
ATCCCACTGCCTAGCCACATTATGACCAAGGGCGCCTTTCTCGGCGGAGCCGATGTGA
AAGAGTGCATCGAGGCCTTCAGCAGCTACATCTGCCCTAGCGATCCTGGCTTCGTGCT
GAACCACGAGATGGAAAGCTGCCTGAGCGGCAACATCTCTCAGTGCCCTAGAACCACC
GTGACCTCCGACATCGTGCCCAGATACGCCTTTGTGAATGGCGGCGTGGTGGCCAACT
GCATCACCACCACCTGTACCTGCAACGGCATCGGCAACCGGATCAACCAGCCTCCAGA
TCAGGGCGTGAAGATTATCACCCACAAAGAGTGTAACACCATCGGCATCAACGGCATG
CTGTTCAATACCAACAAAGAGGGCACCCTGGCCTTCTACACCCCTGACGATATCACCCT
GAACAACAGCGTGGCCCTGGATCCTATCGACATCTCCATCGAGCTGAACAAGCTCAAG
AGCGACCTGGAAGAACTGAAAGAGTGGATCCGGCGGAGCAACCAGAAGCTGGATAGC
ATCGGAAGCTGGCACCAGAGCAGCACCACCATCATCGTGATCCTGATTATGATGATTAT
CCTGTTCATCATCAACATTACCATCATCACGATCGCCATCAAGTACTACCGGATCCAGAA
ACGGAACCGCGTGGACCAGAATGACAAGCCCTACGTGCTGACAAACAAG
>PIV3165_mRNA (SEQ ID NO: 508)
AUGCUGAUCUCCAUCCUGCUGAUCAUCACCACAAUGAUCAUGGCCAGCCACUGCCAG
AUCGACAUCACCAAGCUGCAGCACGUGGGCGUGCUGGUCAAUAGCCCUAAGGGCAU
GAAGAUCAGCCAGAACUUCGAGACACGCUACCUGAUCCUGUCUCUGAUCCCCAAGAU
CGAGGACAGCAACAGCUGCGGCGACCAGCAGAUCAAGCAGUACAAGCGGCUGCUGG
ACAGACUGAUCAUCCCUCUGUACGACGGCCUGCGGCUGCAGAAAGAUGUGAUCGUG
ACCAAUCAAGAGAGCAACGAGAACACAGACCCCAGAACCGAGAGAGGCAGCGGCGGC
GUGAUCGGCACAAUCGCCCUGGGAGUUGCUACAAGCGCCCAGAUUACAGCCGCCGU
GGCUCUGGUGGAAGCCAAGCAGGCCAGAAGCGACAUCGAGAAGCUGAAAGAGGCCA
UCCGGGACACCAACAAGGCCGUGCAGUGCGUGCAAAGCAGCGUGGGCAAUCUGAUC
UGCGCCAUUAAGAGCGUGCAGGACUACGUGAACAAAGAGAUCGUCCCCUCUAUCGCC
AGACUGGGAUGUGAAGCUGCCGGACUGCAGCUGGGAAUUGCCCUGACACAGCACUA
CAGCUGCCUGACCAACAUCUUCGGCGACAACAUCGGCAGCCUGCAAGAGAAGGGCAU
UAAGCUGCAGGGAAUCGCCAGCUGCUACCGCACCAACAUCACCGAGAUCUUCACCAC
CAGCACCGUGGAUAAGUACGACAUCUACGACCUGCUGUUCACCGAGAGCAUCAAAGU
GCGCGUGAUCGACGUGGACCUGAACGACUACAGCAUCACCCUGCAAGUGCGGCUGC
CUCUGCUGACCAGACUGCUGAACACCCAGAUCUACAAGGUGGACAGCAUCUCCUACA
ACAUCCAGAACCGCGAGUGGUACAUCCCACUGCCUAGCCACAUUAUGACCAAGGGCG
CCUUUCUCGGCGGAGCCGAUGUGAAAGAGUGCAUCGAGGCCUUCAGCAGCUACAUC
UGCCCUAGCGAUCCUGGCUUCGUGCUGAACCACGAGAUGGAAAGCUGCCUGAGCGG
CAACAUCUCUCAGUGCCCUAGAACCACCGUGACCUCCGACAUCGUGCCCAGAUACGC
CUUUGUGAAUGGCGGCGUGGUGGCCAACUGCAUCACCACCACCUGUACCUGCAACG
GCAUCGGCAACCGGAUCAACCAGCCUCCAGAUCAGGGCGUGAAGAUUAUCACCCACA
AAGAGUGUAACACCAUCGGCAUCAACGGCAUGCUGUUCAAUACCAACAAAGAGGGCA
CCCUGGCCUUCUACACCCCUGACGAUAUCACCCUGAACAACAGCGUGGCCCUGGAUC
CUAUCGACAUCUCCAUCGAGCUGAACAAGCUCAAGAGCGACCUGGAAGAACUGAAAG
AGUGGAUCCGGCGGAGCAACCAGAAGCUGGAUAGCAUCGGAAGCUGGCACCAGAGC
AGCACCACCAUCAUCGUGAUCCUGAUUAUGAUGAUUAUCCUGUUCAUCAUCAACAUU
ACCAUCAUCACGAUCGCCAUCAAGUACUACCGGAUCCAGAAACGGAACCGCGUGGAC
CAGAAUGACAAGCCCUACGUGCUGACAAACAAG
>PIV3167_DNA (SEQ ID NO: 509)
ATGCTGATCTCCATCCTGCTGATCATCACCACAATGATCATGGCCAGCCACTGCCAGAT
CGACATCACCAAGCTGCAGCACGTGGGCGTGCTGGTCAATAGCCCTAAGGGCATGAAG
ATCAGCCAGAACTTCGAGACACGCTACCTGATCCTGTCTCTGATCCCCAAGATCGAGGA
CAGCAACAGCTGCGGCGACCAGCAGATCAAGCAGTACAAGCGGCTGCTGGACAGACT
GATCATCCCTCTGTACGACGGCCTGCGGCTGCAGAAAGATGTGATCGTGACCAATCAA
GAGAGCAACGAGAACACAGACCCCAGAACCGAGAGATTCTTCGGCGGCGTGATCGGC
ACAATCGCCCTGGGAGTTGCTACAAGCGCCCAGATTACAGCCGCCGTGGCTCTGGTGG
AAGCCAAGCAGGCCAGAAGCGACATCGAGAAGCTGAAAGAGGCCATCCGGGACACCA
ACAAGGCCGTGCAGTGCGTGCAAAGCAGCGTGGGCAATCTGATCTGCGCCATTAAGAG
CGTGCAGGACTACGTGAACAAAGAGATCGTCCCCTCTATCGCCAGACTGGGATGTGAA
GCTGCCGGACTGCAGCTGGGAATTGCCCTGACACAGCACTACAGCGAGCTGACCAACA
TCTTCGGCGACAACATCGGCAGCCTGCAAGAGAAGGGCATTAAGCTGCAGGGAATCGC
CAGCCTGTACCGCACCAACATCACCGAGATCTTCACCACCAGCACCGTGGATAAGTAC
GACATCTACGACCTGCTGTTCACCGAGAGCATCAAAGTGCGCGTGATCGACGTGGACC
TGAACGACTACAGCATCACCCTGCAAGTGCGGCTGCCTCTGCTGACCAGACTGCTGAA
CACCCAGATCTACAAGGTGGACAGCATCTCCTACAACATCCAGAACCGCGAGTGGTAC
ATCCCACTGCCTAGCCACATTATGACCAAGGGCGCCTTTCTCGGCGGAGCCGATGTGA
AAGAGTGCATCGAGGCCTTCAGCAGCTACATCTGCCCTAGCGATCCTGGCTTCGTGCT
GAACCACGAGATGGAAAGCTGCCTGAGCGGCAACATCTCTCAGTGCCCTAGAACCACC
GTGACCTCCGACATCGTGCCCAGATACGCCTTTGTGAATGGCGGCGTGGTGGCCAACT
GCATCACCACCACCTGTACCTGCAACGGCATCGGCAACCGGATCAACCAGCCTCCAGA
TCAGGGCGTGAAGATTATCACCCACAAAGAGTGTAACACCATCGGCATCAACGGCATG
CTGTTCAATACCAACAAAGAGGGCACCCTGGCCTTCTACACCCCTGACGATATCACCCT
GAACAACAGCGTGGCCCTGGATCCTATCGACATCTCCATCGAGCTGAACAAGCTCAAG
AGCGACCTGGAAGAACTGAAAGAGTGGATCCGGCGGAGCAACCAGAAGCTGGATAGC
ATCGGAAGCTGGCACCAGAGCAGCACCACCATCATCGTGATCCTGATTATGATGATTAT
CCTGTTCATCATCAACATTACCATCATCACGATCGCCATCAAGTACTACCGGATCCAGAA
ACGGAACCGCGTGGACCAGAATGACAAGCCCTACGTGCTGACAAACAAG
>PIV3167_mRNA (SEQ ID NO: 510)
AUGCUGAUCUCCAUCCUGCUGAUCAUCACCACAAUGAUCAUGGCCAGCCACUGCCAG
AUCGACAUCACCAAGCUGCAGCACGUGGGCGUGCUGGUCAAUAGCCCUAAGGGCAU
GAAGAUCAGCCAGAACUUCGAGACACGCUACCUGAUCCUGUCUCUGAUCCCCAAGAU
CGAGGACAGCAACAGCUGCGGCGACCAGCAGAUCAAGCAGUACAAGCGGCUGCUGG
ACAGACUGAUCAUCCCUCUGUACGACGGCCUGCGGCUGCAGAAAGAUGUGAUCGUG
ACCAAUCAAGAGAGCAACGAGAACACAGACCCCAGAACCGAGAGAUUCUUCGGCGGC
GUGAUCGGCACAAUCGCCCUGGGAGUUGCUACAAGCGCCCAGAUUACAGCCGCCGU
GGCUCUGGUGGAAGCCAAGCAGGCCAGAAGCGACAUCGAGAAGCUGAAAGAGGCCA
UCCGGGACACCAACAAGGCCGUGCAGUGCGUGCAAAGCAGCGUGGGCAAUCUGAUC
UGCGCCAUUAAGAGCGUGCAGGACUACGUGAACAAAGAGAUCGUCCCCUCUAUCGCC
AGACUGGGAUGUGAAGCUGCCGGACUGCAGCUGGGAAUUGCCCUGACACAGCACUA
CAGCGAGCUGACCAACAUCUUCGGCGACAACAUCGGCAGCCUGCAAGAGAAGGGCAU
UAAGCUGCAGGGAAUCGCCAGCCUGUACCGCACCAACAUCACCGAGAUCUUCACCAC
CAGCACCGUGGAUAAGUACGACAUCUACGACCUGCUGUUCACCGAGAGCAUCAAAGU
GCGCGUGAUCGACGUGGACCUGAACGACUACAGCAUCACCCUGCAAGUGCGGCUGC
CUCUGCUGACCAGACUGCUGAACACCCAGAUCUACAAGGUGGACAGCAUCUCCUACA
ACAUCCAGAACCGCGAGUGGUACAUCCCACUGCCUAGCCACAUUAUGACCAAGGGCG
CCUUUCUCGGCGGAGCCGAUGUGAAAGAGUGCAUCGAGGCCUUCAGCAGCUACAUC
UGCCCUAGCGAUCCUGGCUUCGUGCUGAACCACGAGAUGGAAAGCUGCCUGAGCGG
CAACAUCUCUCAGUGCCCUAGAACCACCGUGACCUCCGACAUCGUGCCCAGAUACGC
CUUUGUGAAUGGCGGCGUGGUGGCCAACUGCAUCACCACCACCUGUACCUGCAACG
GCAUCGGCAACCGGAUCAACCAGCCUCCAGAUCAGGGCGUGAAGAUUAUCACCCACA
AAGAGUGUAACACCAUCGGCAUCAACGGCAUGCUGUUCAAUACCAACAAAGAGGGCA
CCCUGGCCUUCUACACCCCUGACGAUAUCACCCUGAACAACAGCGUGGCCCUGGAUC
CUAUCGACAUCUCCAUCGAGCUGAACAAGCUCAAGAGCGACCUGGAAGAACUGAAAG
AGUGGAUCCGGCGGAGCAACCAGAAGCUGGAUAGCAUCGGAAGCUGGCACCAGAGC
AGCACCACCAUCAUCGUGAUCCUGAUUAUGAUGAUUAUCCUGUUCAUCAUCAACAUU
ACCAUCAUCACGAUCGCCAUCAAGUACUACCGGAUCCAGAAACGGAACCGCGUGGAC
CAGAAUGACAAGCCCUACGUGCUGACAAACAAG
>hMPV170_DNA (SEQ ID NO: 511)
ATGAGCTGGAAAGTCATGATCATCATCAGCCTGCTGATCACCCCTCAGCACGGCCTGAA
AGAGAGCTACCTGGAAGAAAGCTGCAGCACCATCACCGAGGGCTACCTGAGCGTGCTG
AGAACCGGCTGGTACACCAACGTGTTCACCCTGGAAGTGGGCGACGTGGAAAACCTGA
CCTGCACAGATGGCCCCAGCCCCATCAAGACCGAGCTGGATCTGACAAAGAGCGCCCT
GCGCGAGCTGAAAACCGTGTCTGCAGATCAGCTGGCCAGAGAGGAACAGATCGAGAA
CCCCAGACAGAGCAGATTCGTGCTGGGAGCTATCGCCCTGGGAGTTGCTACAGCTGCT
GCTGTGACAGCCGGAATCGCCATTGCCAAGACCATCCGGCTGGAAAGCGAAGTGAACG
CCATCAAGGGCGCACTGAAAACCACCAACGAGGCCGTGTCTACCCTCGGCAACGGTGT
TAGAGTGCTGGCCACAGCCGTGCGGGAACTGAAAGAATTCGTGTCCAAGAACCTGACC
AGCGCCATCAACAAGAACAAGTGCGACATTGCCGACCTGAAGATGGCCGTGTCCTTCA
GCCAGTTCAACCGGCGGTTCCTGAATGTCGTGCGGCAGTTCTCTGACAACGCCGGCAT
CACACCAGCCATTAGCCTGGACCTGATGAACGACGCCGAACTGGCTAGAGCCGTGTCT
TACATGCCTACCTCTGCCGGCCAGATCAAGCTGATGCTGGAAAACAGAGCCATGGTCC
GACGGAAAGGCTTCGGCATCCTGATCGGCGTGTACGGCAGCAGCGTGATCTACATGGT
GCAGCTGCCTATCTTCGGCGTGATCAACACCCCTTGCTGGATCATCAAGGCCGCTCCT
AGCTGCAGCGAGAAGGACGGCAATTACGCCTGCCTGCTGAGAGAGGACCAAGGCTGG
TACTGCAAGAATGCCGGCAGCACCGTGTACTACCCCAACGAGAAGGATTGCGAGACAC
GGGGCGATCACGTGTTCTGTGATACAGCCGCCGGAATCAACGTGGCCGAGCAGAGCA
GAGAGTGCAACATCAACATCAGCACCACAAACTACCCCTGCAAGGTGTCCACCGGCAG
ACACCCTATCAGCATGGTGGCTCTGTCTCCACTGGGAGCCCTGGTGGCTTGTTATAAG
GGCGTGTCCTGTAGCATCGGCAGCAATCAAGTGGGCATCATCAAGCAGCTGCCCAAGG
GCTGCTCCTACATCACCAATCAGGACGCCGACACCGTGACCATCGACAATACCGTGTAT
CAGCTGAGCAAGGTGGAAGGCGAACAGCACGTGATCAAGGGCAGACCTGTGTCCAAC
AGCTTCGACCCCATCAGATTCCCCGAGGACCAGTTCAATGTGGCCCTGGACCAGGTGT
TCGAGAGCATCGAGAATAGCCAGGCTCTGGTGGACCAGTCCAACAAGATCCTGAACTC
CGCCGAGAAGGGCAACACCGGCTTCATCATCGTGATCATCCTGATTGCCGTGCTGGGC
CTGACCATGATCAGCGTGTCCATCATCATTATCATCAAGAAAACGCGGAAGCCCGCCG
GCGCCCCTCCAGAACTTAATGGCGTGACCAACGGCGGCTTCATTCCCCACTCT
>hMPV170_mRNA (SEQ ID NO: 512)
AUGAGCUGGAAAGUCAUGAUCAUCAUCAGCCUGCUGAUCACCCCUCAGCACGGCCUG
AAAGAGAGCUACCUGGAAGAAAGCUGCAGCACCAUCACCGAGGGCUACCUGAGCGUG
CUGAGAACCGGCUGGUACACCAACGUGUUCACCCUGGAAGUGGGCGACGUGGAAAA
CCUGACCUGCACAGAUGGCCCCAGCCCCAUCAAGACCGAGCUGGAUCUGACAAAGAG
CGCCCUGCGCGAGCUGAAAACCGUGUCUGCAGAUCAGCUGGCCAGAGAGGAACAGA
UCGAGAACCCCAGACAGAGCAGAUUCGUGCUGGGAGCUAUCGCCCUGGGAGUUGCU
ACAGCUGCUGCUGUGACAGCCGGAAUCGCCAUUGCCAAGACCAUCCGGCUGGAAAG
CGAAGUGAACGCCAUCAAGGGCGCACUGAAAACCACCAACGAGGCCGUGUCUACCCU
CGGCAACGGUGUUAGAGUGCUGGCCACAGCCGUGCGGGAACUGAAAGAAUUCGUGU
CCAAGAACCUGACCAGCGCCAUCAACAAGAACAAGUGCGACAUUGCCGACCUGAAGA
UGGCCGUGUCCUUCAGCCAGUUCAACCGGCGGUUCCUGAAUGUCGUGCGGCAGUUC
UCUGACAACGCCGGCAUCACACCAGCCAUUAGCCUGGACCUGAUGAACGACGCCGAA
CUGGCUAGAGCCGUGUCUUACAUGCCUACCUCUGCCGGCCAGAUCAAGCUGAUGCU
GGAAAACAGAGCCAUGGUCCGACGGAAAGGCUUCGGCAUCCUGAUCGGCGUGUACG
GCAGCAGCGUGAUCUACAUGGUGCAGCUGCCUAUCUUCGGCGUGAUCAACACCCCU
UGCUGGAUCAUCAAGGCCGCUCCUAGCUGCAGCGAGAAGGACGGCAAUUACGCCUG
CCUGCUGAGAGAGGACCAAGGCUGGUACUGCAAGAAUGCCGGCAGCACCGUGUACU
ACCCCAACGAGAAGGAUUGCGAGACACGGGGCGAUCACGUGUUCUGUGAUACAGCC
GCCGGAAUCAACGUGGCCGAGCAGAGCAGAGAGUGCAACAUCAACAUCAGCACCACA
AACUACCCCUGCAAGGUGUCCACCGGCAGACACCCUAUCAGCAUGGUGGCUCUGUC
UCCACUGGGAGCCCUGGUGGCUUGUUAUAAGGGCGUGUCCUGUAGCAUCGGCAGCA
AUCAAGUGGGCAUCAUCAAGCAGCUGCCCAAGGGCUGCUCCUACAUCACCAAUCAGG
ACGCCGACACCGUGACCAUCGACAAUACCGUGUAUCAGCUGAGCAAGGUGGAAGGC
GAACAGCACGUGAUCAAGGGCAGACCUGUGUCCAACAGCUUCGACCCCAUCAGAUUC
CCCGAGGACCAGUUCAAUGUGGCCCUGGACCAGGUGUUCGAGAGCAUCGAGAAUAG
CCAGGCUCUGGUGGACCAGUCCAACAAGAUCCUGAACUCCGCCGAGAAGGGCAACAC
CGGCUUCAUCAUCGUGAUCAUCCUGAUUGCCGUGCUGGGCCUGACCAUGAUCAGCG
UGUCCAUCAUCAUUAUCAUCAAGAAAACGCGGAAGCCCGCCGGCGCCCCUCCAGAAC
UUAAUGGCGUGACCAACGGCGGCUUCAUUCCCCACUCU
>hMPV171_DNA (SEQ ID NO: 513)
ATGAGCTGGAAAGTCATGATCATCATCAGCCTGCTGATCACCCCTCAGCACGGCCTGAA
AGAGAGCTACCTGGAAGAAAGCTGCAGCACCATCACCGAGGGCTACCTGAGCGTGCTG
AGAACCGGCTGGTACACCAACGTGTTCACCCTGGAAGTGGGCGACGTGGAAAACCTGA
CCTGCACAGATGGCCCCAGCCTGATCAAGACCGAGCTGGATCTGACAAAGAGCGCCCT
GCGCGAGCTGAAAACCGTGTCTGCAGATCAGCTGGCCAGAGAGGAACAGATCGAGAA
CCCCAGACAGAGCAGATTCGTGCTGGGAGCTATCGCCCTGGGAGTTGCTACAGCTGCT
GCTGTGACAGCCGGAATCGCCATTGCCAAGACCATCCGGCTGGAAAGCGAAGTGAACG
CCATCAAGGGCGCACTGAAAACCACCAACGAGGCCGTGTCTACCCTCGGCAACGGTGT
TAGAGTGCTGGCCACAGCCGTGCGGGAACTGAAAGAATTCGTGTCCAAGAACCTGACC
AGCGCCATCAACAAGAACAAGTGCGACATTGCCGACCCCAAGATGGCCGTGTCCTTCA
GCCAGTTCAACCGGCGGTTCCTGAATGTCGTGCGGCAGTTCTCTGACAACGCCGGCAT
CACACCAGCCATTAGCCTGGACCTGATGAACGACGCCGAACTGGCTAGAGCCGTGTCT
TACATGCCTACCTCTGCCGGCCAGATCAAGCTGATGCTGGAAAACAGAGCCATGGTCC
GACGGAAAGGCTTCGGCATCCTGATCGGCGTGTACGGCAGCAGCGTGATCTACATGGT
GCAGCTGCCTATCTTCGGCGTGATCAACACCCCTTGCTGGATCATCAAGGCCGCTCCT
AGCTGCAGCGAGAAGGACGGCAATTACGCCTGCCTGCTGAGAGAGGACCAAGGCTGG
TACTGCAAGAATGCCGGCAGCACCGTGTACTACCCCAACGAGAAGGATTGCGAGACAC
GGGGCGATCACGTGTTCTGTGATACAGCCGCCGGAATCAACGTGGCCGAGCAGAGCA
GAGAGTGCAACATCAACATCAGCACCACAAACTACCCCTGCAAGGTGTCCACCGGCAG
ACACCCTATCAGCATGGTGGCTCTGTCTCCACTGGGAGCCCTGGTGGCTTGTTATAAG
GGCGTGTCCTGTAGCATCGGCAGCAATCAAGTGGGCATCATCAAGCAGCTGCCCAAGG
GCTGCTCCTACATCACCAATCAGGACGCCGACACCGTGACCATCGACAATACCGTGTAT
CAGCTGAGCAAGGTGGAAGGCGAACAGCACGTGATCAAGGGCAGACCTGTGTCCAAC
AGCTTCGACCCCATCAGATTCCCCGAGGACCAGTTCAATGTGGCCCTGGACCAGGTGT
TCGAGAGCATCGAGAATAGCCAGGCTCTGGTGGACCAGTCCAACAAGATCCTGAACTC
CGCCGAGAAGGGCAACACCGGCTTCATCATCGTGATCATCCTGATTGCCGTGCTGGGC
CTGACCATGATCAGCGTGTCCATCATCATTATCATCAAGAAAACGCGGAAGCCCGCCG
GCGCCCCTCCAGAACTTAATGGCGTGACCAACGGCGGCTTCATTCCCCACTCT
>hMPV171_mRNA (SEQ ID NO: 514)
AUGAGCUGGAAAGUCAUGAUCAUCAUCAGCCUGCUGAUCACCCCUCAGCACGGCCUG
AAAGAGAGCUACCUGGAAGAAAGCUGCAGCACCAUCACCGAGGGCUACCUGAGCGUG
CUGAGAACCGGCUGGUACACCAACGUGUUCACCCUGGAAGUGGGCGACGUGGAAAA
CCUGACCUGCACAGAUGGCCCCAGCCUGAUCAAGACCGAGCUGGAUCUGACAAAGAG
CGCCCUGCGCGAGCUGAAAACCGUGUCUGCAGAUCAGCUGGCCAGAGAGGAACAGA
UCGAGAACCCCAGACAGAGCAGAUUCGUGCUGGGAGCUAUCGCCCUGGGAGUUGCU
ACAGCUGCUGCUGUGACAGCCGGAAUCGCCAUUGCCAAGACCAUCCGGCUGGAAAG
CGAAGUGAACGCCAUCAAGGGCGCACUGAAAACCACCAACGAGGCCGUGUCUACCCU
CGGCAACGGUGUUAGAGUGCUGGCCACAGCCGUGCGGGAACUGAAAGAAUUCGUGU
CCAAGAACCUGACCAGCGCCAUCAACAAGAACAAGUGCGACAUUGCCGACCCCAAGA
UGGCCGUGUCCUUCAGCCAGUUCAACCGGCGGUUCCUGAAUGUCGUGCGGCAGUUC
UCUGACAACGCCGGCAUCACACCAGCCAUUAGCCUGGACCUGAUGAACGACGCCGAA
CUGGCUAGAGCCGUGUCUUACAUGCCUACCUCUGCCGGCCAGAUCAAGCUGAUGCU
GGAAAACAGAGCCAUGGUCCGACGGAAAGGCUUCGGCAUCCUGAUCGGCGUGUACG
GCAGCAGCGUGAUCUACAUGGUGCAGCUGCCUAUCUUCGGCGUGAUCAACACCCCU
UGCUGGAUCAUCAAGGCCGCUCCUAGCUGCAGCGAGAAGGACGGCAAUUACGCCUG
CCUGCUGAGAGAGGACCAAGGCUGGUACUGCAAGAAUGCCGGCAGCACCGUGUACU
ACCCCAACGAGAAGGAUUGCGAGACACGGGGCGAUCACGUGUUCUGUGAUACAGCC
GCCGGAAUCAACGUGGCCGAGCAGAGCAGAGAGUGCAACAUCAACAUCAGCACCACA
AACUACCCCUGCAAGGUGUCCACCGGCAGACACCCUAUCAGCAUGGUGGCUCUGUC
UCCACUGGGAGCCCUGGUGGCUUGUUAUAAGGGCGUGUCCUGUAGCAUCGGCAGCA
AUCAAGUGGGCAUCAUCAAGCAGCUGCCCAAGGGCUGCUCCUACAUCACCAAUCAGG
ACGCCGACACCGUGACCAUCGACAAUACCGUGUAUCAGCUGAGCAAGGUGGAAGGC
GAACAGCACGUGAUCAAGGGCAGACCUGUGUCCAACAGCUUCGACCCCAUCAGAUUC
CCCGAGGACCAGUUCAAUGUGGCCCUGGACCAGGUGUUCGAGAGCAUCGAGAAUAG
CCAGGCUCUGGUGGACCAGUCCAACAAGAUCCUGAACUCCGCCGAGAAGGGCAACAC
CGGCUUCAUCAUCGUGAUCAUCCUGAUUGCCGUGCUGGGCCUGACCAUGAUCAGCG
UGUCCAUCAUCAUUAUCAUCAAGAAAACGCGGAAGCCCGCCGGCGCCCCUCCAGAAC
UUAAUGGCGUGACCAACGGCGGCUUCAUUCCCCACUCU
>hMPV172_DNA (SEQ ID NO: 515)
ATGAGCTGGAAAGTCATGATCATCATCAGCCTGCTGATCACCCCTCAGCACGGCCTGAA
AGAGAGCTACCTGGAAGAAAGCTGCAGCACCATCACCGAGGGCTACCTGAGCGTGCTG
AGAACCGGCTGGTACACCAACGTGTTCACCCTGGAAGTGGGCGACGTGGAAAACCTGA
CCTGCACAGATGGCCCCAGCCTGATCAAGACCGAGCTGGATCTGACAAAGAGCGCCCT
GCGCGAGCTGAAAACCGTGTCTGCAGATCAGCTGGCCAGAGAGGAACAGATCGAGAA
CCCCAGACGGCGGAGATTCGTGCTGGGAGCTATCGCCCTGGGAGTTGCTACAGCTGC
TGCTGTGACAGCCGGAATCGCCATTGCCAAGACCATCCGGCTGGAAAGCGAAGTGAAC
GCCATCAAGGGCGCACTGAAAACCACCAACGAGGCCGTGTCTACCCTCGGCAACGGT
GTTAGAGTGCTGGCCACAGCCGTGCGGGAACTGAAAGAATTCGTGTCCAAGAACCTGA
CCAGCGCCATCAACAAGAACAAGTGCGACATTGCCGACCCCAAGATGGCCGTGTCCTT
CAGCCAGTTCAACCGGCGGTTCCTGAATGTCGTGCGGCAGTTCTCTGACAACGCCGGC
ATCACACCAGCCATTAGCCTGGACCTGATGAACGACGCCGAACTGGCTAGAGCCGTGT
CTTACATGCCTACCTCTGCCGGCCAGATCAAGCTGATGCTGGAAAACAGAGCCATGGT
CCGACGGAAAGGCTTCGGCATCCTGATCGGCGTGTACGGCAGCAGCGTGATCTACATG
GTGCAGCTGCCTATCTTCGGCGTGATCAACACCCCTTGCTGGATCATCAAGGCCGCTC
CTAGCTGCAGCGAGAAGGACGGCAATTACGCCTGCCTGCTGAGAGAGGACCAAGGCT
GGTACTGCAAGAATGCCGGCAGCACCGTGTACTACCCCAACGAGAAGGATTGCGAGAC
ACGGGGCGATCACGTGTTCTGTGATACAGCCGCCGGAATCAACGTGGCCGAGCAGAG
CAGAGAGTGCAACATCAACATCAGCACCACAAACTACCCCTGCAAGGTGTCCACCGGC
AGACACCCTATCAGCATGGTGGCTCTGTCTCCACTGGGAGCCCTGGTGGCTTGTTATAA
GGGCGTGTCCTGTAGCATCGGCAGCAATCAAGTGGGCATCATCAAGCAGCTGCCCAAG
GGCTGCTCCTACATCACCAATCAGGACGCCGACACCGTGACCATCGACAATACCGTGT
ATCAGCTGAGCAAGGTGGAAGGCGAACAGCACGTGATCAAGGGCAGACCTGTGTCCAA
CAGCTTCGACCCCATCAGATTCCCCGAGGACCAGTTCAATGTGGCCCTGGACCAGGTG
TTCGAGAGCATCGAGAATAGCCAGGCTCTGGTGGACCAGTCCAACAAGATCCTGAACT
CCGCCGAGAAGGGCAACACCGGCTTCATCATCGTGATCATCCTGATTGCCGTGCTGGG
CCTGACCATGATCAGCGTGTCCATCATCATTATCATCAAGAAAACGCGGAAGCCCGCCG
GCGCCCCTCCAGAACTTAATGGCGTGACCAACGGCGGCTTCATTCCCCACTCT
>hMPV172_mRNA (SEQ ID NO: 516)
AUGAGCUGGAAAGUCAUGAUCAUCAUCAGCCUGCUGAUCACCCCUCAGCACGGCCUG
AAAGAGAGCUACCUGGAAGAAAGCUGCAGCACCAUCACCGAGGGCUACCUGAGCGUG
CUGAGAACCGGCUGGUACACCAACGUGUUCACCCUGGAAGUGGGCGACGUGGAAAA
CCUGACCUGCACAGAUGGCCCCAGCCUGAUCAAGACCGAGCUGGAUCUGACAAAGAG
CGCCCUGCGCGAGCUGAAAACCGUGUCUGCAGAUCAGCUGGCCAGAGAGGAACAGA
UCGAGAACCCCAGACGGCGGAGAUUCGUGCUGGGAGCUAUCGCCCUGGGAGUUGCU
ACAGCUGCUGCUGUGACAGCCGGAAUCGCCAUUGCCAAGACCAUCCGGCUGGAAAG
CGAAGUGAACGCCAUCAAGGGCGCACUGAAAACCACCAACGAGGCCGUGUCUACCCU
CGGCAACGGUGUUAGAGUGCUGGCCACAGCCGUGCGGGAACUGAAAGAAUUCGUGU
CCAAGAACCUGACCAGCGCCAUCAACAAGAACAAGUGCGACAUUGCCGACCCCAAGA
UGGCCGUGUCCUUCAGCCAGUUCAACCGGCGGUUCCUGAAUGUCGUGCGGCAGUUC
UCUGACAACGCCGGCAUCACACCAGCCAUUAGCCUGGACCUGAUGAACGACGCCGAA
CUGGCUAGAGCCGUGUCUUACAUGCCUACCUCUGCCGGCCAGAUCAAGCUGAUGCU
GGAAAACAGAGCCAUGGUCCGACGGAAAGGCUUCGGCAUCCUGAUCGGCGUGUACG
GCAGCAGCGUGAUCUACAUGGUGCAGCUGCCUAUCUUCGGCGUGAUCAACACCCCU
UGCUGGAUCAUCAAGGCCGCUCCUAGCUGCAGCGAGAAGGACGGCAAUUACGCCUG
CCUGCUGAGAGAGGACCAAGGCUGGUACUGCAAGAAUGCCGGCAGCACCGUGUACU
ACCCCAACGAGAAGGAUUGCGAGACACGGGGCGAUCACGUGUUCUGUGAUACAGCC
GCCGGAAUCAACGUGGCCGAGCAGAGCAGAGAGUGCAACAUCAACAUCAGCACCACA
AACUACCCCUGCAAGGUGUCCACCGGCAGACACCCUAUCAGCAUGGUGGCUCUGUC
UCCACUGGGAGCCCUGGUGGCUUGUUAUAAGGGCGUGUCCUGUAGCAUCGGCAGCA
AUCAAGUGGGCAUCAUCAAGCAGCUGCCCAAGGGCUGCUCCUACAUCACCAAUCAGG
ACGCCGACACCGUGACCAUCGACAAUACCGUGUAUCAGCUGAGCAAGGUGGAAGGC
GAACAGCACGUGAUCAAGGGCAGACCUGUGUCCAACAGCUUCGACCCCAUCAGAUUC
CCCGAGGACCAGUUCAAUGUGGCCCUGGACCAGGUGUUCGAGAGCAUCGAGAAUAG
CCAGGCUCUGGUGGACCAGUCCAACAAGAUCCUGAACUCCGCCGAGAAGGGCAACAC
CGGCUUCAUCAUCGUGAUCAUCCUGAUUGCCGUGCUGGGCCUGACCAUGAUCAGCG
UGUCCAUCAUCAUUAUCAUCAAGAAAACGCGGAAGCCCGCCGGCGCCCCUCCAGAAC
UUAAUGGCGUGACCAACGGCGGCUUCAUUCCCCACUCU
>hMPV173_DNA (SEQ ID NO: 517)
ATGAGCTGGAAAGTCATGATCATCATCAGCCTGCTGATCACCCCTCAGCACGGCCTGAA
AGAGAGCTACCTGGAAGAAAGCTGCAGCACCATCACCGAGGGCTACCTGAGCGTGCTG
AGAACCGGCTGGTACACCAACGTGTTCACCCTGGAAGTGGGCGACGTGGAAAACCTGA
CCTGCACAGATGGCCCCAGCCTGATCAAGACCGAGCTGGATCTGACAAAGAGCGCCCT
GCGCGAGCTGAAAACCGTGTCTGCAGATCAGCTGGCCAGAGAGGAACAGATCGAGAA
CCCCAGACAGAGCAGATTCGTGCTGGGAGCTATCGCCCTGGGAGTTGCTACAGCTGCT
GCTGTGACAGCCGGAATCGCCATTGCCAAGACCATCCGGCTGGAAAGCGAAGTGAACG
CCATCAAGGGCTGCCTGAAAACCACCAACGAGGCCGTGTGCACCCTCGGCAACGGTGT
TAGAGTGCTGGCCACAGCCGTGCGGGAACTGAAAGAATTCGTGTCCAAGAACCTGACC
AGCGCCATCAACAAGAACAAGTGCGACATTGCCGACCCCAAGATGGCCGTGTCCTTCA
GCCAGTTCAACCGGCGGTTCCTGAATGTCGTGCGGCAGTTCTCTGACAACGCCGGCAT
CACACCAGCCATTAGCCTGGACCTGATGAACGACGCCGAACTGGCTAGAGCCGTGTCT
TACATGCCTACCTCTGCCGGCCAGATCAAGCTGATGCTGGAAAACAGAGCCATGGTCC
GACGGAAAGGCTTCGGCATCCTGATCGGCGTGTACGGCAGCAGCGTGATCTACATGGT
GCAGCTGCCTATCTTCGGCGTGATCAACACCCCTTGCTGGATCATCAAGGCCGCTCCT
AGCTGCAGCGAGAAGGACGGCAATTACGCCTGCCTGCTGAGAGAGGACCAAGGCTGG
TACTGCAAGAATGCCGGCAGCACCGTGTACTACCCCAACGAGAAGGATTGCGAGACAC
GGGGCGATCACGTGTTCTGTGATACAGCCGCCGGAATCAACGTGGCCGAGCAGAGCA
GAGAGTGCAACATCAACATCAGCACCACAAACTACCCCTGCAAGGTGTCCACCGGCAG
ACACCCTATCAGCATGGTGGCTCTGTCTCCACTGGGAGCCCTGGTGGCTTGTTATAAG
GGCGTGTCCTGTAGCATCGGCAGCAATCAAGTGGGCATCATCAAGCAGCTGCCCAAGG
GCTGCTCCTACATCACCAATCAGGACGCCGACACCGTGACCATCGACAATACCGTGTAT
CAGCTGAGCAAGGTGGAAGGCGAACAGCACGTGATCAAGGGCAGACCTGTGTCCAAC
AGCTTCGACCCCATCAGATTCCCCGAGGACCAGTTCAATGTGGCCCTGGACCAGGTGT
TCGAGAGCATCGAGAATAGCCAGGCTCTGGTGGACCAGTCCAACAAGATCCTGAACTC
CGCCGAGAAGGGCAACACCGGCTTCATCATCGTGATCATCCTGATTGCCGTGCTGGGC
CTGACCATGATCAGCGTGTCCATCATCATTATCATCAAGAAAACGCGGAAGCCCGCCG
GCGCCCCTCCAGAACTTAATGGCGTGACCAACGGCGGCTTCATTCCCCACTCT
>hMPV173_mRNA (SEQ ID NO: 518)
AUGAGCUGGAAAGUCAUGAUCAUCAUCAGCCUGCUGAUCACCCCUCAGCACGGCCUG
AAAGAGAGCUACCUGGAAGAAAGCUGCAGCACCAUCACCGAGGGCUACCUGAGCGUG
CUGAGAACCGGCUGGUACACCAACGUGUUCACCCUGGAAGUGGGCGACGUGGAAAA
CCUGACCUGCACAGAUGGCCCCAGCCUGAUCAAGACCGAGCUGGAUCUGACAAAGAG
CGCCCUGCGCGAGCUGAAAACCGUGUCUGCAGAUCAGCUGGCCAGAGAGGAACAGA
UCGAGAACCCCAGACAGAGCAGAUUCGUGCUGGGAGCUAUCGCCCUGGGAGUUGCU
ACAGCUGCUGCUGUGACAGCCGGAAUCGCCAUUGCCAAGACCAUCCGGCUGGAAAG
CGAAGUGAACGCCAUCAAGGGCUGCCUGAAAACCACCAACGAGGCCGUGUGCACCCU
CGGCAACGGUGUUAGAGUGCUGGCCACAGCCGUGCGGGAACUGAAAGAAUUCGUGU
CCAAGAACCUGACCAGCGCCAUCAACAAGAACAAGUGCGACAUUGCCGACCCCAAGA
UGGCCGUGUCCUUCAGCCAGUUCAACCGGCGGUUCCUGAAUGUCGUGCGGCAGUUC
UCUGACAACGCCGGCAUCACACCAGCCAUUAGCCUGGACCUGAUGAACGACGCCGAA
CUGGCUAGAGCCGUGUCUUACAUGCCUACCUCUGCCGGCCAGAUCAAGCUGAUGCU
GGAAAACAGAGCCAUGGUCCGACGGAAAGGCUUCGGCAUCCUGAUCGGCGUGUACG
GCAGCAGCGUGAUCUACAUGGUGCAGCUGCCUAUCUUCGGCGUGAUCAACACCCCU
UGCUGGAUCAUCAAGGCCGCUCCUAGCUGCAGCGAGAAGGACGGCAAUUACGCCUG
CCUGCUGAGAGAGGACCAAGGCUGGUACUGCAAGAAUGCCGGCAGCACCGUGUACU
ACCCCAACGAGAAGGAUUGCGAGACACGGGGCGAUCACGUGUUCUGUGAUACAGCC
GCCGGAAUCAACGUGGCCGAGCAGAGCAGAGAGUGCAACAUCAACAUCAGCACCACA
AACUACCCCUGCAAGGUGUCCACCGGCAGACACCCUAUCAGCAUGGUGGCUCUGUC
UCCACUGGGAGCCCUGGUGGCUUGUUAUAAGGGCGUGUCCUGUAGCAUCGGCAGCA
AUCAAGUGGGCAUCAUCAAGCAGCUGCCCAAGGGCUGCUCCUACAUCACCAAUCAGG
ACGCCGACACCGUGACCAUCGACAAUACCGUGUAUCAGCUGAGCAAGGUGGAAGGC
GAACAGCACGUGAUCAAGGGCAGACCUGUGUCCAACAGCUUCGACCCCAUCAGAUUC
CCCGAGGACCAGUUCAAUGUGGCCCUGGACCAGGUGUUCGAGAGCAUCGAGAAUAG
CCAGGCUCUGGUGGACCAGUCCAACAAGAUCCUGAACUCCGCCGAGAAGGGCAACAC
CGGCUUCAUCAUCGUGAUCAUCCUGAUUGCCGUGCUGGGCCUGACCAUGAUCAGCG
UGUCCAUCAUCAUUAUCAUCAAGAAAACGCGGAAGCCCGCCGGCGCCCCUCCAGAAC
UUAAUGGCGUGACCAACGGCGGCUUCAUUCCCCACUCU
>hMPV174_DNA (SEQ ID NO: 519)
ATGAGCTGGAAAGTCATGATCATCATCAGCCTGCTGATCACCCCTCAGCACGGCCTGAA
AGAGAGCTACCTGGAAGAAAGCTGCAGCACCATCACCGAGGGCTACCTGAGCGTGCTG
AGAACCGGCTGGTACACCAACGTGTTCACCCTGGAAGTGGGCGACGTGGAAAACCTGA
CCTGCACAGATGGCCCCAGCCTGATCAAGACCGAGCTGGATCTGACAAAGAGCGCCCT
GCGCGAGCTGAAAACCGTGTCTGCAGATCAGCTGGCCAGAGAGGAACAGATCGAGAA
CCCCAGACGGCGGAGATTCGTGCTGGGAGCTATCGCCCTGGGAGTTGCTACAGCTGC
TGCTGTGACAGCCGGAATCGCCATTGCCAAGACCATCCGGCTGGAAAGCGAAGTGAAC
GCCATCAAGGGCTGCCTGAAAACCACCAACGAGGCCGTGTGCACCCTCGGCAACGGT
GTTAGAGTGCTGGCCACAGCCGTGCGGGAACTGAAAGAATTCGTGTCCAAGAACCTGA
CCAGCGCCATCAACAAGAACAAGTGCGACATTGCCGACCCCAAGATGGCCGTGTCCTT
CAGCCAGTTCAACCGGCGGTTCCTGAATGTCGTGCGGCAGTTCTCTGACAACGCCGGC
ATCACACCAGCCATTAGCCTGGACCTGATGAACGACGCCGAACTGGCTAGAGCCGTGT
CTTACATGCCTACCTCTGCCGGCCAGATCAAGCTGATGCTGGAAAACAGAGCCATGGT
CCGACGGAAAGGCTTCGGCATCCTGATCGGCGTGTACGGCAGCAGCGTGATCTACATG
GTGCAGCTGCCTATCTTCGGCGTGATCAACACCCCTTGCTGGATCATCAAGGCCGCTC
CTAGCTGCAGCGAGAAGGACGGCAATTACGCCTGCCTGCTGAGAGAGGACCAAGGCT
GGTACTGCAAGAATGCCGGCAGCACCGTGTACTACCCCAACGAGAAGGATTGCGAGAC
ACGGGGCGATCACGTGTTCTGTGATACAGCCGCCGGAATCAACGTGGCCGAGCAGAG
CAGAGAGTGCAACATCAACATCAGCACCACAAACTACCCCTGCAAGGTGTCCACCGGC
AGACACCCTATCAGCATGGTGGCTCTGTCTCCACTGGGAGCCCTGGTGGCTTGTTATAA
GGGCGTGTCCTGTAGCATCGGCAGCAATCAAGTGGGCATCATCAAGCAGCTGCCCAAG
GGCTGCTCCTACATCACCAATCAGGACGCCGACACCGTGACCATCGACAATACCGTGT
ATCAGCTGAGCAAGGTGGAAGGCGAACAGCACGTGATCAAGGGCAGACCTGTGTCCAA
CAGCTTCGACCCCATCAGATTCCCCGAGGACCAGTTCAATGTGGCCCTGGACCAGGTG
TTCGAGAGCATCGAGAATAGCCAGGCTCTGGTGGACCAGTCCAACAAGATCCTGAACT
CCGCCGAGAAGGGCAACACCGGCTTCATCATCGTGATCATCCTGATTGCCGTGCTGGG
CCTGACCATGATCAGCGTGTCCATCATCATTATCATCAAGAAAACGCGGAAGCCCGCCG
GCGCCCCTCCAGAACTTAATGGCGTGACCAACGGCGGCTTCATTCCCCACTCT
>hMPV174_mRNA (SEQ ID NO: 520)
AUGAGCUGGAAAGUCAUGAUCAUCAUCAGCCUGCUGAUCACCCCUCAGCACGGCCUG
AAAGAGAGCUACCUGGAAGAAAGCUGCAGCACCAUCACCGAGGGCUACCUGAGCGUG
CUGAGAACCGGCUGGUACACCAACGUGUUCACCCUGGAAGUGGGCGACGUGGAAAA
CCUGACCUGCACAGAUGGCCCCAGCCUGAUCAAGACCGAGCUGGAUCUGACAAAGAG
CGCCCUGCGCGAGCUGAAAACCGUGUCUGCAGAUCAGCUGGCCAGAGAGGAACAGA
UCGAGAACCCCAGACGGCGGAGAUUCGUGCUGGGAGCUAUCGCCCUGGGAGUUGCU
ACAGCUGCUGCUGUGACAGCCGGAAUCGCCAUUGCCAAGACCAUCCGGCUGGAAAG
CGAAGUGAACGCCAUCAAGGGCUGCCUGAAAACCACCAACGAGGCCGUGUGCACCCU
CGGCAACGGUGUUAGAGUGCUGGCCACAGCCGUGCGGGAACUGAAAGAAUUCGUGU
CCAAGAACCUGACCAGCGCCAUCAACAAGAACAAGUGCGACAUUGCCGACCCCAAGA
UGGCCGUGUCCUUCAGCCAGUUCAACCGGCGGUUCCUGAAUGUCGUGCGGCAGUUC
UCUGACAACGCCGGCAUCACACCAGCCAUUAGCCUGGACCUGAUGAACGACGCCGAA
CUGGCUAGAGCCGUGUCUUACAUGCCUACCUCUGCCGGCCAGAUCAAGCUGAUGCU
GGAAAACAGAGCCAUGGUCCGACGGAAAGGCUUCGGCAUCCUGAUCGGCGUGUACG
GCAGCAGCGUGAUCUACAUGGUGCAGCUGCCUAUCUUCGGCGUGAUCAACACCCCU
UGCUGGAUCAUCAAGGCCGCUCCUAGCUGCAGCGAGAAGGACGGCAAUUACGCCUG
CCUGCUGAGAGAGGACCAAGGCUGGUACUGCAAGAAUGCCGGCAGCACCGUGUACU
ACCCCAACGAGAAGGAUUGCGAGACACGGGGCGAUCACGUGUUCUGUGAUACAGCC
GCCGGAAUCAACGUGGCCGAGCAGAGCAGAGAGUGCAACAUCAACAUCAGCACCACA
AACUACCCCUGCAAGGUGUCCACCGGCAGACACCCUAUCAGCAUGGUGGCUCUGUC
UCCACUGGGAGCCCUGGUGGCUUGUUAUAAGGGCGUGUCCUGUAGCAUCGGCAGCA
AUCAAGUGGGCAUCAUCAAGCAGCUGCCCAAGGGCUGCUCCUACAUCACCAAUCAGG
ACGCCGACACCGUGACCAUCGACAAUACCGUGUAUCAGCUGAGCAAGGUGGAAGGC
GAACAGCACGUGAUCAAGGGCAGACCUGUGUCCAACAGCUUCGACCCCAUCAGAUUC
CCCGAGGACCAGUUCAAUGUGGCCCUGGACCAGGUGUUCGAGAGCAUCGAGAAUAG
CCAGGCUCUGGUGGACCAGUCCAACAAGAUCCUGAACUCCGCCGAGAAGGGCAACAC
CGGCUUCAUCAUCGUGAUCAUCCUGAUUGCCGUGCUGGGCCUGACCAUGAUCAGCG
UGUCCAUCAUCAUUAUCAUCAAGAAAACGCGGAAGCCCGCCGGCGCCCCUCCAGAAC
UUAAUGGCGUGACCAACGGCGGCUUCAUUCCCCACUCU
>hMPV175_DNA (SEQ ID NO: 521)
ATGAGCTGGAAAGTCATGATCATCATCAGCCTGCTGATCACCCCTCAGCACGGCCTGAA
AGAGAGCTACCTGGAAGAAAGCTGCAGCACCATCACCGAGGGCTACCTGAGCGTGCTG
AGAACCGGCTGGTACACCAACGTGTTCATCCTGGAAGTGGGCGACGTGGAAAACCTGA
CCTGCACAGATGGCCCCAGCCTGATCAAGACCGAGCTGGATCTGACAAAGAGCGCCCT
GCGCGAGCTGAAAACCGTGTCTGCAGATCAGCTGGCCAGAGAGGAACAGATCGAGAA
CCCCAGACAGAGCAGATTCGTGCTGGGAGCTATCGCCCTGGGAGTTGCTACAGCTGCT
GCTGTGACAGCCGGAATCGCCATTGCCAAGACCATCCGGCTGGAAAGCGAAGTGAACG
CCATCAAGGGCGCACTGAAAACCACCAACGAGGCCGTGTCTACCCTCGGCAACGGTGT
TAGAGTGCTGGCCACAGCCGTGCGGGAACTGAAAGAATTCGTGTCCAAGAACCTGACC
AGCGCCATCAACAAGAACAAGTGCGACATTGCCGACCTGAAGATGGCCGTGTCCTTCA
GCCAGTTCAACCGGCGGTTCCTGAATGTCGTGCGGCAGTTCTCTGACAACGCCGGCAT
CACACCAGCCATTAGCCTGGACCTGATGAACGACGCCGAACTGGCTAGAGCCGTGTCT
TACATGCCTACCTCTGCCGGCCAGATCAAGCTGATGCTGGAAAACAGAGCCATGGTCC
GACGGAAAGGCTTCGGCATCCTGATCGGCGTGTACGGCAGCAGCGTGATCTACATGGT
GCAGCTGCCTATCTTCGGCGTGATCAACACCCCTTGCTGGATCATCAAGGCCGCTCCT
AGCTGCAGCGAGAAGGACGGCAATTACGCCTGCCTGCTGAGAGAGGACCAAGGCTGG
TACTGCAAGAATGCCGGCAGCACCGTGTACTACCCCAACGAGAAGGATTGCGAGACAC
GGGGCGATCACGTGTTCTGTGATACAGCCGCCGGAATCAACGTGGCCGAGCAGAGCA
GAGAGTGCAACATCAACATCAGCACCACAAACTACCCCTGCAAGGTGTCCACCGGCAG
ACACCCTATCAGCATGGTGGCTCTGTCTCCACTGGGAGCCCTGGTGGCTTGTTATAAG
GGCGTGTCCTGTAGCATCGGCAGCAATCAAGTGGGCATCATCAAGCAGCTGCCCAAGG
GCTGCTCCTACATCACCAATCAGGACGCCGACACCGTGACCATCGACAATACCGTGTAT
CAGCTGAGCAAGGTGGAAGGCGAACAGCACGTGATCAAGGGCAGACCTGTGTCCAAC
AGCTTCGACCCCATCAGATTCCCCGAGGACCAGTTCAATGTGGCCCTGGACCAGGTGT
TCGAGAGCATCGAGAATAGCCAGGCTCTGGTGGACCAGTCCAACAAGATCCTGAACTC
CGCCGAGAAGGGCAACACCGGCTTCATCATCGTGATCATCCTGATTGCCGTGCTGGGC
CTGACCATGATCAGCGTGTCCATCATCATTATCATCAAGAAAACGCGGAAGCCCGCCG
GCGCCCCTCCAGAACTTAATGGCGTGACCAACGGCGGCTTCATTCCCCACTCT
>hMPV175_mRNA (SEQ ID NO: 522)
AUGAGCUGGAAAGUCAUGAUCAUCAUCAGCCUGCUGAUCACCCCUCAGCACGGCCUG
AAAGAGAGCUACCUGGAAGAAAGCUGCAGCACCAUCACCGAGGGCUACCUGAGCGUG
CUGAGAACCGGCUGGUACACCAACGUGUUCAUCCUGGAAGUGGGCGACGUGGAAAA
CCUGACCUGCACAGAUGGCCCCAGCCUGAUCAAGACCGAGCUGGAUCUGACAAAGAG
CGCCCUGCGCGAGCUGAAAACCGUGUCUGCAGAUCAGCUGGCCAGAGAGGAACAGA
UCGAGAACCCCAGACAGAGCAGAUUCGUGCUGGGAGCUAUCGCCCUGGGAGUUGCU
ACAGCUGCUGCUGUGACAGCCGGAAUCGCCAUUGCCAAGACCAUCCGGCUGGAAAG
CGAAGUGAACGCCAUCAAGGGCGCACUGAAAACCACCAACGAGGCCGUGUCUACCCU
CGGCAACGGUGUUAGAGUGCUGGCCACAGCCGUGCGGGAACUGAAAGAAUUCGUGU
CCAAGAACCUGACCAGCGCCAUCAACAAGAACAAGUGCGACAUUGCCGACCUGAAGA
UGGCCGUGUCCUUCAGCCAGUUCAACCGGCGGUUCCUGAAUGUCGUGCGGCAGUUC
UCUGACAACGCCGGCAUCACACCAGCCAUUAGCCUGGACCUGAUGAACGACGCCGAA
CUGGCUAGAGCCGUGUCUUACAUGCCUACCUCUGCCGGCCAGAUCAAGCUGAUGCU
GGAAAACAGAGCCAUGGUCCGACGGAAAGGCUUCGGCAUCCUGAUCGGCGUGUACG
GCAGCAGCGUGAUCUACAUGGUGCAGCUGCCUAUCUUCGGCGUGAUCAACACCCCU
UGCUGGAUCAUCAAGGCCGCUCCUAGCUGCAGCGAGAAGGACGGCAAUUACGCCUG
CCUGCUGAGAGAGGACCAAGGCUGGUACUGCAAGAAUGCCGGCAGCACCGUGUACU
ACCCCAACGAGAAGGAUUGCGAGACACGGGGCGAUCACGUGUUCUGUGAUACAGCC
GCCGGAAUCAACGUGGCCGAGCAGAGCAGAGAGUGCAACAUCAACAUCAGCACCACA
AACUACCCCUGCAAGGUGUCCACCGGCAGACACCCUAUCAGCAUGGUGGCUCUGUC
UCCACUGGGAGCCCUGGUGGCUUGUUAUAAGGGCGUGUCCUGUAGCAUCGGCAGCA
AUCAAGUGGGCAUCAUCAAGCAGCUGCCCAAGGGCUGCUCCUACAUCACCAAUCAGG
ACGCCGACACCGUGACCAUCGACAAUACCGUGUAUCAGCUGAGCAAGGUGGAAGGC
GAACAGCACGUGAUCAAGGGCAGACCUGUGUCCAACAGCUUCGACCCCAUCAGAUUC
CCCGAGGACCAGUUCAAUGUGGCCCUGGACCAGGUGUUCGAGAGCAUCGAGAAUAG
CCAGGCUCUGGUGGACCAGUCCAACAAGAUCCUGAACUCCGCCGAGAAGGGCAACAC
CGGCUUCAUCAUCGUGAUCAUCCUGAUUGCCGUGCUGGGCCUGACCAUGAUCAGCG
UGUCCAUCAUCAUUAUCAUCAAGAAAACGCGGAAGCCCGCCGGCGCCCCUCCAGAAC
UUAAUGGCGUGACCAACGGCGGCUUCAUUCCCCACUCU
>hMPV176_DNA (SEQ ID NO: 102)
ATGAGCTGGAAAGTCATGATCATCATCAGCCTGCTGATCACCCCTCAGCACGGCCTGAA
AGAGAGCTACCTGGAAGAAAGCTGCAGCACCATCACCGAGGGCTACCTGAGCGTGCTG
AGAACCGGCTGGTACACCAACGTGTTCACCCTGGAAGTGGGCGACGTGGAAAACCTGA
CCTGCACAGATGGCCCCAGCCTGATCAAGACCGAGCTGGATCTGACAAAGAGCGCCCT
GCGCGAGCTGAAAACCGTGTCTGCAGATCAGCTGGCCAGAGAGGAACAGATCGAGAA
CCCCAGACAGAGCAGATTCGTGCTGGGAGCTATCGCCCTGGGAGTTGCTACAGCTGCT
GCTGTGACAGCCGGAATCGCCATTGCCAAGACCATCCGGCTGGAAAGCGAAGTGAACG
CCATCAAGGGCGCACTGAAAACCACCAACGAGGCCGTGTCTACCCTCGGCAACGGTGT
TAGAGTGCTGGCCACAGCCGTGCGGGAACTGAAAGAATTCGTGTCCAAGAACCTGACC
AGCGCCATCAACAAGAACAAGTGCGACATTGCCGACCTGAAGATGGCCGTGTCCTTCA
GCCAGTTCAACCGGCGGTTCCTGAATGTCGTGCGGCAGTTCTCTGACAACGCCGGCAT
CACACCAGCCATTAGCCTGGACCTGATGAACGACGCCGAACTGGCTAGAGCCGTGTCT
TACATGCCTACCTCTGCCGGCCAGATCAAGCTGATGCTGGAAAACAGAGCCATGGTCC
GACGGAAAGGCTTCGGCATCCTGATCGGCGTGTACGGCAGCAGCGTGATCTACATGGT
GCAGCTGCCTATCTTCGGCGTGATCAACACCCCTTGCTGGATCATCAAGGCCGCTCCT
AGCTGCAGCGAGAAGGACGGCAATTACGCCTGCCTGCTGAGAGAGGACCAAGGCTGG
TACTGCAAGAATGCCGGCAGCACCGTGTACTACCCCAACGAGAAGGATTGCGAGACAC
GGGGCGATCACGTGTTCTGTGATACAGCCGCCGGAATCAACGTGGCCGAGCAGAGCA
GAGAGTGCAACATCAACATCAGCACCACAAACTACCCCTGCAAGGTGTCCATCGGCAG
ACACCCTATCAGCATGGTGGCTCTGTCTCCACTGGGAGCCCTGGTGGCTTGTTATAAG
GGCGTGTCCTGTAGCATCGGCAGCAATCAAGTGGGCATCATCAAGCAGCTGCCCAAGG
GCTGCTCCTACATCACCAATCAGGACGCCGACACCGTGACCATCGACAATACCGTGTAT
CAGCTGAGCAAGGTGGAAGGCGAACAGCACGTGATCAAGGGCAGACCTGTGTCCAAC
AGCTTCGACCCCATCAGATTCCCCGAGGACCAGTTCAATGTGGCCCTGGACCAGGTGT
TCGAGAGCATCGAGAATAGCCAGGCTCTGGTGGACCAGTCCAACAAGATCCTGAACTC
CGCCGAGAAGGGCAACACCGGCTTCATCATCGTGATCATCCTGATTGCCGTGCTGGGC
CTGACCATGATCAGCGTGTCCATCATCATTATCATCAAGAAAACGCGGAAGCCCGCCG
GCGCCCCTCCAGAACTTAATGGCGTGACCAACGGCGGCTTCATTCCCCACTCT
>hMPV176 mRNA (SEQ ID NO: 103)
AUGAGCUGGAAAGUCAUGAUCAUCAUCAGCCUGCUGAUCACCCCUCAGCACGGCCUG
AAAGAGAGCUACCUGGAAGAAAGCUGCAGCACCAUCACCGAGGGCUACCUGAGCGUG
CUGAGAACCGGCUGGUACACCAACGUGUUCACCCUGGAAGUGGGCGACGUGGAAAA
CCUGACCUGCACAGAUGGCCCCAGCCUGAUCAAGACCGAGCUGGAUCUGACAAAGAG
CGCCCUGCGCGAGCUGAAAACCGUGUCUGCAGAUCAGCUGGCCAGAGAGGAACAGA
UCGAGAACCCCAGACAGAGCAGAUUCGUGCUGGGAGCUAUCGCCCUGGGAGUUGCU
ACAGCUGCUGCUGUGACAGCCGGAAUCGCCAUUGCCAAGACCAUCCGGCUGGAAAG
CGAAGUGAACGCCAUCAAGGGCGCACUGAAAACCACCAACGAGGCCGUGUCUACCCU
CGGCAACGGUGUUAGAGUGCUGGCCACAGCCGUGCGGGAACUGAAAGAAUUCGUGU
CCAAGAACCUGACCAGCGCCAUCAACAAGAACAAGUGCGACAUUGCCGACCUGAAGA
UGGCCGUGUCCUUCAGCCAGUUCAACCGGCGGUUCCUGAAUGUCGUGCGGCAGUUC
UCUGACAACGCCGGCAUCACACCAGCCAUUAGCCUGGACCUGAUGAACGACGCCGAA
CUGGCUAGAGCCGUGUCUUACAUGCCUACCUCUGCCGGCCAGAUCAAGCUGAUGCU
GGAAAACAGAGCCAUGGUCCGACGGAAAGGCUUCGGCAUCCUGAUCGGCGUGUACG
GCAGCAGCGUGAUCUACAUGGUGCAGCUGCCUAUCUUCGGCGUGAUCAACACCCCU
UGCUGGAUCAUCAAGGCCGCUCCUAGCUGCAGCGAGAAGGACGGCAAUUACGCCUG
CCUGCUGAGAGAGGACCAAGGCUGGUACUGCAAGAAUGCCGGCAGCACCGUGUACU
ACCCCAACGAGAAGGAUUGCGAGACACGGGGCGAUCACGUGUUCUGUGAUACAGCC
GCCGGAAUCAACGUGGCCGAGCAGAGCAGAGAGUGCAACAUCAACAUCAGCACCACA
AACUACCCCUGCAAGGUGUCCAUCGGCAGACACCCUAUCAGCAUGGUGGCUCUGUC
UCCACUGGGAGCCCUGGUGGCUUGUUAUAAGGGCGUGUCCUGUAGCAUCGGCAGCA
AUCAAGUGGGCAUCAUCAAGCAGCUGCCCAAGGGCUGCUCCUACAUCACCAAUCAGG
ACGCCGACACCGUGACCAUCGACAAUACCGUGUAUCAGCUGAGCAAGGUGGAAGGC
GAACAGCACGUGAUCAAGGGCAGACCUGUGUCCAACAGCUUCGACCCCAUCAGAUUC
CCCGAGGACCAGUUCAAUGUGGCCCUGGACCAGGUGUUCGAGAGCAUCGAGAAUAG
CCAGGCUCUGGUGGACCAGUCCAACAAGAUCCUGAACUCCGCCGAGAAGGGCAACAC
CGGCUUCAUCAUCGUGAUCAUCCUGAUUGCCGUGCUGGGCCUGACCAUGAUCAGCG
UGUCCAUCAUCAUUAUCAUCAAGAAAACGCGGAAGCCCGCCGGCGCCCCUCCAGAAC
UUAAUGGCGUGACCAACGGCGGCUUCAUUCCCCACUCU
>hMPV177_DNA (SEQ ID NO: 104)
ATGAGCTGGAAAGTCATGATCATCATCAGCCTGCTGATCACCCCTCAGCACGGCCTGAA
AGAGAGCTACCTGGAAGAAAGCTGCAGCACCATCACCGAGGGCTACCTGAGCGTGCTG
AGAACCGGCTGGTACACCAACGTGTTCATCCTGGAAGTGGGCGACGTGGAAAACCTGA
CCTGCACAGATGGCCCCAGCCTGATCAAGACCGAGCTGGATCTGACAAAGAGCGCCCT
GCGCGAGCTGAAAACCGTGTCTGCAGATCAGCTGGCCAGAGAGGAACAGATCGAGAA
CCCCAGACAGAGCAGATTCGTGCTGGGAGCTATCGCCCTGGGAGTTGCTACAGCTGCT
GCTGTGACAGCCGGAATCGCCATTGCCAAGACCATCCGGCTGGAAAGCGAAGTGAACG
CCATCAAGGGCGCACTGAAAACCACCAACGAGGCCGTGTCTACCCTCGGCAACGGTGT
TAGAGTGCTGGCCACAGCCGTGCGGGAACTGAAAGAATTCGTGTCCAAGAACCTGACC
AGCGCCATCAACAAGAACAAGTGCGACATTGCCGACCTGAAGATGGCCGTGTCCTTCA
GCCAGTTCAACCGGCGGTTCCTGAATGTCGTGCGGCAGTTCTCTGACAACGCCGGCAT
CACACCAGCCATTAGCCTGGACCTGATGAACGACGCCGAACTGGCTAGAGCCGTGTCT
TACATGCCTACCTCTGCCGGCCAGATCAAGCTGATGCTGGAAAACAGAGCCATGGTCC
GACGGAAAGGCTTCGGCATCCTGATCGGCGTGTACGGCAGCAGCGTGATCTACATGGT
GCAGCTGCCTATCTTCGGCGTGATCAACACCCCTTGCTGGATCATCAAGGCCGCTCCT
AGCTGCAGCGAGAAGGACGGCAATTACGCCTGCCTGCTGAGAGAGGACCAAGGCTGG
TACTGCAAGAATGCCGGCAGCACCGTGTACTACCCCAACGAGAAGGATTGCGAGACAC
GGGGCGATCACGTGTTCTGTGATACAGCCGCCGGAATCAACGTGGCCGAGCAGAGCA
GAGAGTGCAACATCAACATCAGCACCACAAACTACCCCTGCAAGGTGTCCATCGGCAG
ACACCCTATCAGCATGGTGGCTCTGTCTCCACTGGGAGCCCTGGTGGCTTGTTATAAG
GGCGTGTCCTGTAGCATCGGCAGCAATCAAGTGGGCATCATCAAGCAGCTGCCCAAGG
GCTGCTCCTACATCACCAATCAGGACGCCGACACCGTGACCATCGACAATACCGTGTAT
CAGCTGAGCAAGGTGGAAGGCGAACAGCACGTGATCAAGGGCAGACCTGTGTCCAAC
AGCTTCGACCCCATCAGATTCCCCGAGGACCAGTTCAATGTGGCCCTGGACCAGGTGT
TCGAGAGCATCGAGAATAGCCAGGCTCTGGTGGACCAGTCCAACAAGATCCTGAACTC
CGCCGAGAAGGGCAACACCGGCTTCATCATCGTGATCATCCTGATTGCCGTGCTGGGC
CTGACCATGATCAGCGTGTCCATCATCATTATCATCAAGAAAACGCGGAAGCCCGCCG
GCGCCCCTCCAGAACTTAATGGCGTGACCAACGGCGGCTTCATTCCCCACTCT
>hMPV177 mRNA (SEQ ID NO: 105)
AUGAGCUGGAAAGUCAUGAUCAUCAUCAGCCUGCUGAUCACCCCUCAGCACGGCCUG
AAAGAGAGCUACCUGGAAGAAAGCUGCAGCACCAUCACCGAGGGCUACCUGAGCGUG
CUGAGAACCGGCUGGUACACCAACGUGUUCAUCCUGGAAGUGGGCGACGUGGAAAA
CCUGACCUGCACAGAUGGCCCCAGCCUGAUCAAGACCGAGCUGGAUCUGACAAAGAG
CGCCCUGCGCGAGCUGAAAACCGUGUCUGCAGAUCAGCUGGCCAGAGAGGAACAGA
UCGAGAACCCCAGACAGAGCAGAUUCGUGCUGGGAGCUAUCGCCCUGGGAGUUGCU
ACAGCUGCUGCUGUGACAGCCGGAAUCGCCAUUGCCAAGACCAUCCGGCUGGAAAG
CGAAGUGAACGCCAUCAAGGGCGCACUGAAAACCACCAACGAGGCCGUGUCUACCCU
CGGCAACGGUGUUAGAGUGCUGGCCACAGCCGUGCGGGAACUGAAAGAAUUCGUGU
CCAAGAACCUGACCAGCGCCAUCAACAAGAACAAGUGCGACAUUGCCGACCUGAAGA
UGGCCGUGUCCUUCAGCCAGUUCAACCGGCGGUUCCUGAAUGUCGUGCGGCAGUUC
UCUGACAACGCCGGCAUCACACCAGCCAUUAGCCUGGACCUGAUGAACGACGCCGAA
CUGGCUAGAGCCGUGUCUUACAUGCCUACCUCUGCCGGCCAGAUCAAGCUGAUGCU
GGAAAACAGAGCCAUGGUCCGACGGAAAGGCUUCGGCAUCCUGAUCGGCGUGUACG
GCAGCAGCGUGAUCUACAUGGUGCAGCUGCCUAUCUUCGGCGUGAUCAACACCCCU
UGCUGGAUCAUCAAGGCCGCUCCUAGCUGCAGCGAGAAGGACGGCAAUUACGCCUG
CCUGCUGAGAGAGGACCAAGGCUGGUACUGCAAGAAUGCCGGCAGCACCGUGUACU
ACCCCAACGAGAAGGAUUGCGAGACACGGGGCGAUCACGUGUUCUGUGAUACAGCC
GCCGGAAUCAACGUGGCCGAGCAGAGCAGAGAGUGCAACAUCAACAUCAGCACCACA
AACUACCCCUGCAAGGUGUCCAUCGGCAGACACCCUAUCAGCAUGGUGGCUCUGUC
UCCACUGGGAGCCCUGGUGGCUUGUUAUAAGGGCGUGUCCUGUAGCAUCGGCAGCA
AUCAAGUGGGCAUCAUCAAGCAGCUGCCCAAGGGCUGCUCCUACAUCACCAAUCAGG
ACGCCGACACCGUGACCAUCGACAAUACCGUGUAUCAGCUGAGCAAGGUGGAAGGC
GAACAGCACGUGAUCAAGGGCAGACCUGUGUCCAACAGCUUCGACCCCAUCAGAUUC
CCCGAGGACCAGUUCAAUGUGGCCCUGGACCAGGUGUUCGAGAGCAUCGAGAAUAG
CCAGGCUCUGGUGGACCAGUCCAACAAGAUCCUGAACUCCGCCGAGAAGGGCAACAC
CGGCUUCAUCAUCGUGAUCAUCCUGAUUGCCGUGCUGGGCCUGACCAUGAUCAGCG
UGUCCAUCAUCAUUAUCAUCAAGAAAACGCGGAAGCCCGCCGGCGCCCCUCCAGAAC
UUAAUGGCGUGACCAACGGCGGCUUCAUUCCCCACUCU
SEQ ID No: 523 Amino Acid Sequence of the CDRH 1 domain (KABAT) of Antibody hMPV-2
mAb
SYGIS
SEQ ID No: 524 Amino Acid Sequence of the CDRH1 domain (Chothia) of Antibody hMPV-2
mAb
GYTFTSY
SEQ ID No: 525 Amino Acid Sequence of the CDRH2 domain (KABAT) of Antibody hMPV-2
mAb
WISGYNGNTNYAQTFQG
SEQ ID No: 526 Amino Acid Sequence of the CDRH2 domain (Chothia) of Antibody hMPV-2
mAb
SGYNGN
SEQ ID No: 527 Amino Acid Sequence of the CDRH3 domain (KABAT) of Antibody hMPV-2
mAb
DRYYASGSYNGMDV
SEQ ID No: 528 Amino Acid Sequence of the CDRH3 domain (Chothia) of Antibody hMPV-2
mAb
DRYYASGSYNGMDV
SEQ ID No: 529 Amino Acid Sequence of the CDRL1 domain of Antibody hMPV-2 mAb
SGSSSNVGSSSVY
SEQ ID No: 530 Amino Acid Sequence of the CDRL2 domain of Antibody hMPV-2 mAb
RNNQRPS
SEQ ID No: 531 Amino Acid Sequence of the CDRL3 domain of Antibody hMPV-2 mAb
AAWDDSLSGVV
SEQ ID No: 532 Amino Acid Sequence of the Heavy Chain of Antibody hMPV-2 mAb
QVQLQQSGAEVKKPGASVKVSCKASGYTFTSYGISWVRQAPGQGLEWMGWISGYNGNTN
YAQTFQGTFTMTTDTSTSTAYMELRRLRSGDTAVYYCARDRYYASGSYNGMDVWGQGTT
VTVSSASTKGPSVFPLAPSSKSTSGGTAALGCLVKDYFPEPVTVSWNSGALTSGVHTFPAV
LQSSGLYSLSSVVTVPSSSLGTQTYICNVNHKPSNTKVDKKVEPKSCDKTHTCPPCPAPELL
GGPSVFLFPPKPKDTLMISRTPEVTCVVVDVSHEDPEVKFNWYVDGVEVHNAKTKPREEQ
YNSTYRVVSVLTVLHQDWLNGKEYKCKVSNKALPAPIEKTISKAKGQPREPQVYTLPPSREE
MTKNQVSLTCLVKGFYPSDIAVEWESNGQPENNYKTTPPVLDSDGSFFLYSKLTVDKSRW
QQGNVFSCSVMHEALHNHYTQKSLSLSPGK
SEQ ID No: 533 Amino Acid Sequence of the Light Chain of Antibody hMPV-2 mAb
QSALTQPPSASGAPGQRVTVSCSGSSSNVGSSSVYWYQQLPGTAPKLLIYRNNQRPSGVP
DRFSGSKSGTSASLAISGLRSEDEADYYCAAWDDSLSGWVFGGGTKLTVLGQPKAAPSVT
LFPPSSEELQANKATLVCLISDFYPGAVTVAWKADSSPVKAGVETTTPSKQSNNKYAASSYL
SLTPEQWKSHRSYSCQVTHEGSTVEKTVAPTECS
SEQ ID No: 534 Amino Acid Sequence of the CDRH1 domain (KABAT) of Antibody PIV1-8
mAb
NYVIS
SEQ ID No: 535 Amino Acid Sequence of the CDRH1 domain (Chothia) of Antibody PIV1-8
mAb
GGTFSNY
SEQ ID No: 536 Amino Acid Sequence of the CDRH2 domain (KABAT) of Antibody PIV1-8
mAb
GIIPVFATAKYAQKFQG
SEQ ID No: 537 Amino Acid Sequence of the CDRH2 domain (Chothia) of Antibody PIV1-8
mAb
IPVFAT
SEQ ID No: 538 Amino Acid Sequence of the CDRH3 domain (KABAT) of Antibody PIV1-8
mAb
VDSGGTWWDAFAI
SEQ ID No: 539 Amino Acid Sequence of the CDRH3 domain (Chothia) of Antibody PIV1-8
mAb
VDSGGTWWDAFAI
SEQ ID No: 540 Amino Acid Sequence of the CDRL1 domain of Antibody PIV1-8 mAb
TGTSSDVGGYNYVS
SEQ ID No: 541 Amino Acid Sequence of the CDRL2 domain of Antibody PIV1-8 mAb
DVTKRPS
SEQ ID No: 542 Amino Acid Sequence of the CDRL3 domain of Antibody PIV1-8 mAb
CSYAGSYTLV
SEQ ID No: 543 Amino Acid Sequence of the Heavy Chain of Antibody PIV1-8 mAb
QVQLVQSGAEVKKPGSSVKVSCEASGGTFSNYVISWVRQAPGQGLEWMGGIIPVFATAKY
AQKFQGRVTIIADQSTNTAYLELSSLRSDDTAVYFCASVDSGGTWWDAFAIWGQGTMVTVS
SASTKGPSVFPLAPSSKSTSGGTAALGCLVKDYFPEPVTVSWNSGALTSGVHTFPAVLQSS
GLYSLSSVVTVPSSSLGTQTYICNVNHKPSNTKVDKKVEPKSCDKTHTCPPCPAPELLGGP
SVFLFPPKPKDTLMISRTPEVTCVVVDVSHEDPEVKFNWYVDGVEVHNAKTKPREEQYNST
YRVVSVLTVLHQDWLNGKEYKCKVSNKALPAPIEKTISKAKGQPREPQVYTLPPSREEMTK
NQVSLTCLVKGFYPSDIAVEWESNGQPENNYKTTPPVLDSDGSFFLYSKLTVDKSRWQQG
NVFSCSVMHEALHNHYTQKSLSLSPGK
SEQ ID No: 544 Amino Acid Sequence of the Light Chain of Antibody PIV1-8 mAb
QAVVTQPRSVSGSPGQAVTISCTGTSSDVGGYNYVSWYQHHPGKAPKLIIYDVTKRPSGVP
DRFSGSKSGNTASLTISGLQTEYEADYYCCSYAGSYTLVFGGGTKLTVLGQPKAAPSVTLF
PPSSEELQANKATLVCLISDFYPGAVTVAWKADSSPVKAGVETTTPSKQSNNKYAASSYLSL
TPEQWKSHRSYSCQVTHEGSTVEKTVAPTECS
SEQ ID No: 545 DNA Sequence Encoding the Heavy Chain Variable domain of Antibody
hMPV-2 mAb
CAGGTGCAGCTACAGCAGTCTGGAGCTGAGGTGAAGAAGCCTGGGGCCTCTGTGAAG
GTCTCATGCAAGGCTTCTGGTTACACCTTTACCAGCTATGGTATCAGCTGGGTGCGCCA
GGCCCCTGGACAAGGGCTTGAGTGGATGGGGTGGATCAGCGGTTACAATGGAAACAC
AAACTATGCACAGACGTTCCAGGGCACTTTCACCATGACCACAGACACATCCACGAGCA
CAGCCTACATGGAGCTGAGGAGGCTGCGATCTGGCGACACGGCCGTATACTACTGTGC
GAGAGATCGCTACTATGCTTCGGGGAGTTACAACGGTATGGACGTCTGGGGCCAAGGG
ACCACGGTCACTGTCTCCTCA
SEQ ID No: 546 DNA Sequence Encoding the Light Chain Variable domain of Antibody
hMPV-2 mAb
CAGTCTGCCCTGACTCAGCCACCCTCAGCGTCTGGGGCCCCCGGGCAGAGGGTCACC
GTCTCTTGTTCTGGAAGCAGCTCCAACGTCGGAAGTAGTTCTGTATACTGGTACCAGCA
GCTGCCAGGAACGGCCCCCAAACTCCTCATCTATAGGAATAATCAGCGGCCCTCAGGG
GTCCCTGACCGATTCTCTGGCTCCAAGTCTGGCACCTCAGCCTCCCTGGCCATCAGTG
GGCTCCGGTCCGAGGATGAGGCTGATTATTACTGTGCAGCATGGGATGACAGCCTGAG
TGGTTGGGTCTTCGGCGGAGGGACCAAGCTGACCGTCCTA
SEQ ID No: 547 DNA Sequence Encoding the Heavy Chain of Antibody hMPV-2 mAb
CAGGTGCAGCTACAGCAGTCTGGAGCTGAGGTGAAGAAGCCTGGGGCCTCTGTGAAG
GTCTCATGCAAGGCTTCTGGTTACACCTTTACCAGCTATGGTATCAGCTGGGTGCGCCA
GGCCCCTGGACAAGGGCTTGAGTGGATGGGGTGGATCAGCGGTTACAATGGAAACAC
AAACTATGCACAGACGTTCCAGGGCACTTTCACCATGACCACAGACACATCCACGAGCA
CAGCCTACATGGAGCTGAGGAGGCTGCGATCTGGCGACACGGCCGTATACTACTGTGC
GAGAGATCGCTACTATGCTTCGGGGAGTTACAACGGTATGGACGTCTGGGGCCAAGGG
ACCACGGTCACTGTCTCCTCAGCGTCGACCAAGGGCCCATCGGTCTTCCCCCTGGCAC
CCTCCTCCAAGAGCACCTCTGGGGGCACAGCGGCCCTGGGCTGCCTGGTCAAGGACT
ACTTCCCCGAACCGGTGACGGTGTCGTGGAACTCAGGCGCCCTGACCAGCGGCGTGC
ACACCTTCCCGGCTGTCCTACAGTCCTCAGGACTCTACTCCCTCAGCAGCGTGGTGAC
CGTGCCCTCCAGCAGCTTGGGCACCCAGACCTACATCTGCAACGTGAATCACAAGCCC
AGCAACACCAAGGTGGACAAGAAAGTTGAGCCCAAATCTTGTGACAAAACTCACACATG
CCCACCGTGCCCAGCACCTGAACTCCTGGGGGGACCGTCAGTCTTCCTCTTCCCCCCA
AAACCCAAGGACACCCTCATGATCTCCCGGACCCCTGAGGTCACATGCGTGGTGGTGG
ACGTGAGCCACGAAGACCCTGAGGTCAAGTTCAACTGGTACGTGGACGGCGTGGAGG
TGCATAATGCCAAGACAAAGCCGCGGGAGGAGCAGTACAACAGCACGTACCGTGTGGT
CAGCGTCCTCACCGTCCTGCACCAGGACTGGCTGAATGGCAAGGAGTACAAGTGCAAG
GTCTCCAACAAAGCCCTCCCAGCCCCCATCGAGAAAACCATCTCCAAAGCCAAAGGGC
AGCCCCGAGAACCACAGGTGTACACCCTGCCCCCATCCCGGGAGGAGATGACCAAGA
ACCAGGTCAGCCTGACCTGCCTGGTCAAAGGCTTCTATCCCAGCGACATCGCCGTGGA
GTGGGAGAGCAATGGGCAGCCGGAGAACAACTACAAGACCACGCCTCCCGTGCTGGA
CTCCGACGGCTCCTTCTTCCTCTATAGCAAGCTCACCGTGGACAAGAGCAGGTGGCAG
CAGGGGAACGTCTTCTCATGCTCCGTGATGCATGAGGCTCTGCACAACCACTACACGC
AGAAGAGCCTCTCCCTGTCCCCGGGTAAA
SEQ ID No: 548 DNA Sequence Encoding the Light Chain of Antibody hMPV-2 mAb
CAGTCTGCCCTGACTCAGCCACCCTCAGCGTCTGGGGCCCCCGGGCAGAGGGTCACC
GTCTCTTGTTCTGGAAGCAGCTCCAACGTCGGAAGTAGTTCTGTATACTGGTACCAGCA
GCTGCCAGGAACGGCCCCCAAACTCCTCATCTATAGGAATAATCAGCGGCCCTCAGGG
GTCCCTGACCGATTCTCTGGCTCCAAGTCTGGCACCTCAGCCTCCCTGGCCATCAGTG
GGCTCCGGTCCGAGGATGAGGCTGATTATTACTGTGCAGCATGGGATGACAGCCTGAG
TGGTTGGGTCTTCGGCGGAGGGACCAAGCTGACCGTCCTAGGTCAGCCCAAGGCTGC
CCCCTCGGTCACTCTGTTCCCGCCCTCCTCTGAGGAGCTTCAAGCCAACAAGGCCACA
CTGGTGTGTCTCATAAGTGACTTCTACCCGGGAGCCGTGACAGTGGCCTGGAAGGCAG
ATAGCAGCCCCGTCAAGGCGGGAGTGGAGACCACCACACCCTCCAAACAAAGCAACAA
CAAGTACGCGGCCAGCAGCTATCTGAGCCTGACGCCTGAGCAGTGGAAGTCCCACAGA
AGCTACAGCTGCCAGGTCACGCATGAAGGGAGCACCGTGGAGAAGACAGTGGCCCCT
ACAGAATGTTCA
SEQ ID No: 549 DNA Sequence Encoding the Heavy Chain Variable domain of Antibody
PIV1-8 mAb
CAGGTCCAGCTTGTACAGTCTGGGGCTGAGGTGAAGAAGCCTGGGTCCTCGGTGAAG
GTCTCCTGCGAGGCTTCTGGAGGCACCTTCAGCAATTACGTTATCAGTTGGGTGCGAC
AGGCCCCTGGACAAGGGCTTGAGTGGATGGGAGGGATCATCCCAGTCTTTGCTACAGC
AAAATATGCACAGAAGTTCCAGGGCAGAGTCACGATTATCGCGGACCAATCCACGAATA
CAGCCTACTTGGAGCTGAGCAGCCTGAGATCTGACGACACTGCCGTTTATTTCTGTGCG
AGCGTGGATAGTGGTGGTACTTGGTGGGATGCTTTTGCTATCTGGGGCCAAGGGACAA
TGGTCACCGTCTCCTCA
SEQ ID No: 550 DNA Sequence Encoding the Light Chain Variable domain of Antibody
PIV1-8 mAb
CAGGCTGTGGTGACCCAGCCTCGCTCAGTGTCCGGGTCTCCTGGACAGGCAGTCACC
ATCTCCTGCACTGGAACCAGCAGTGATGTTGGTGGTTATAACTATGTCTCCTGGTACCA
GCACCACCCAGGCAAAGCCCCCAAACTCATCATTTATGATGTCACTAAGCGGCCCTCAG
GGGTCCCTGATCGGTTCTCTGGCTCCAAGTCTGGCAACACGGCCTCCCTGACCATCTC
TGGGCTCCAGACTGAGTATGAGGCTGATTATTACTGCTGCTCATATGCAGGCAGCTACA
CTTTGGTTTTCGGCGGAGGGACCAAGCTGACCGTCCTA
SEQ ID No: 551 DNA Sequence Encoding the Heavy Chain of Antibody PIV1-8 mAb
CAGGTCCAGCTTGTACAGTCTGGGGCTGAGGTGAAGAAGCCTGGGTCCTCGGTGAAG
GTCTCCTGCGAGGCTTCTGGAGGCACCTTCAGCAATTACGTTATCAGTTGGGTGCGAC
AGGCCCCTGGACAAGGGCTTGAGTGGATGGGAGGGATCATCCCAGTCTTTGCTACAGC
AAAATATGCACAGAAGTTCCAGGGCAGAGTCACGATTATCGCGGACCAATCCACGAATA
CAGCCTACTTGGAGCTGAGCAGCCTGAGATCTGACGACACTGCCGTTTATTTCTGTGCG
AGCGTGGATAGTGGTGGTACTTGGTGGGATGCTTTTGCTATCTGGGGCCAAGGGACAA
TGGTCACCGTCTCCTCAGCGTCGACCAAGGGCCCATCGGTCTTCCCCCTGGCACCCTC
CTCCAAGAGCACCTCTGGGGGCACAGCGGCCCTGGGCTGCCTGGTCAAGGACTACTT
CCCCGAACCGGTGACGGTGTCGTGGAACTCAGGCGCCCTGACCAGCGGCGTGCACAC
CTTCCCGGCTGTCCTACAGTCCTCAGGACTCTACTCCCTCAGCAGCGTGGTGACCGTG
CCCTCCAGCAGCTTGGGCACCCAGACCTACATCTGCAACGTGAATCACAAGCCCAGCA
ACACCAAGGTGGACAAGAAAGTTGAGCCCAAATCTTGTGACAAAACTCACACATGCCCA
CCGTGCCCAGCACCTGAACTCCTGGGGGGACCGTCAGTCTTCCTCTTCCCCCCAAAAC
CCAAGGACACCCTCATGATCTCCCGGACCCCTGAGGTCACATGCGTGGTGGTGGACGT
GAGCCACGAAGACCCTGAGGTCAAGTTCAACTGGTACGTGGACGGCGTGGAGGTGCA
TAATGCCAAGACAAAGCCGCGGGAGGAGCAGTACAACAGCACGTACCGTGTGGTCAGC
GTCCTCACCGTCCTGCACCAGGACTGGCTGAATGGCAAGGAGTACAAGTGCAAGGTCT
CCAACAAAGCCCTCCCAGCCCCCATCGAGAAAACCATCTCCAAAGCCAAAGGGCAGCC
CCGAGAACCACAGGTGTACACCCTGCCCCCATCCCGGGAGGAGATGACCAAGAACCA
GGTCAGCCTGACCTGCCTGGTCAAAGGCTTCTATCCCAGCGACATCGCCGTGGAGTGG
GAGAGCAATGGGCAGCCGGAGAACAACTACAAGACCACGCCTCCCGTGCTGGACTCC
GACGGCTCCTTCTTCCTCTATAGCAAGCTCACCGTGGACAAGAGCAGGTGGCAGCAGG
GGAACGTCTTCTCATGCTCCGTGATGCATGAGGCTCTGCACAACCACTACACGCAGAA
GAGCCTCTCCCTGTCCCCGGGTAAA
SEQ ID No: 552 DNA Sequence Encoding the Light Chain of Antibody PIV1-8 mAb
CAGGCTGTGGTGACCCAGCCTCGCTCAGTGTCCGGGTCTCCTGGACAGGCAGTCACC
ATCTCCTGCACTGGAACCAGCAGTGATGTTGGTGGTTATAACTATGTCTCCTGGTACCA
GCACCACCCAGGCAAAGCCCCCAAACTCATCATTTATGATGTCACTAAGCGGCCCTCAG
GGGTCCCTGATCGGTTCTCTGGCTCCAAGTCTGGCAACACGGCCTCCCTGACCATCTC
TGGGCTCCAGACTGAGTATGAGGCTGATTATTACTGCTGCTCATATGCAGGCAGCTACA
CTTTGGTTTTCGGCGGAGGGACCAAGCTGACCGTCCTAGGTCAGCCCAAGGCTGCCCC
CTCGGTCACTCTGTTCCCGCCCTCCTCTGAGGAGCTTCAAGCCAACAAGGCCACACTG
GTGTGTCTCATAAGTGACTTCTACCCGGGAGCCGTGACAGTGGCCTGGAAGGCAGATA
GCAGCCCCGTCAAGGCGGGAGTGGAGACCACCACACCCTCCAAACAAAGCAACAACAA
GTACGCGGCCAGCAGCTATCTGAGCCTGACGCCTGAGCAGTGGAAGTCCCACAGAAG
CTACAGCTGCCAGGTCACGCATGAAGGGAGCACCGTGGAGAAGACAGTGGCCCCTAC
AGAATGTTCATGAG

1. A mutant of a wild-type PIV1 F protein, which mutant comprises a F1 polypeptide and a F2 polypeptide, wherein the mutant comprises at least one amino acid mutation relative to the amino acid sequence of the wild-type PIV1 F protein, and wherein the amino acid mutation is selected from the group consisting of:
(1) at least one engineered disulfide bond mutation;
(2) at least one cavity filling mutation;
(3) at least one proline substitution mutation;
(4) at least one glycine replacement mutation; and,
(5) a cleavage site mutation.

2. The mutant according to claim 1 wherein the mutant comprises an engineered disulfide mutation.

3. The mutant according to claim 1 or 2 wherein the engineered disulfide mutation is Q92C-G134C.

4. The mutant according to any one of claims 1 to 3, wherein the mutant comprises a cavity filling mutation.

5. The mutant according to any one of claims 1 to 4, wherein the cavity filling mutation is selected from T198A, Q92A, Q92L, A466L, A466V, A4661, S473V, S473L, S4731, S473A, A480L and A480V.

6. The mutant according to claim 5, wherein the cavity filling mutation is A466L.

7. The mutant according to claim 5, wherein the cavity filling mutation is S473L.

8. The mutant according to claim 5, wherein the cavity filling mutation is A480L.

9. The mutant according to any one of claims 1 to 4, wherein the mutant comprises two or three cavity filling mutations selected from T198A, Q92A, Q92L, A466L, A466V, A4661, S473V, S473L, S4731, S473A, A480L and A480V.

10. The mutant according to claim 9, wherein the cavity filling mutations are A466L and S473L.

11. The mutant according to claim 9 or 10 further comprising the cavity filling mutation A480L or A480V.

12. The mutant according to any one of claims 1 to 11, wherein the mutant comprises a proline substitution mutation.

13. The mutant according to claim 12, wherein the proline substitution mutation is A128P.

14. The mutant according to any one of claims 1 to 13, wherein the mutant comprises a glycine replacement mutation.

15. The mutant according to claim 14, wherein the glycine replacement mutation is G134 Aor G134L.

16. The mutant according to claim 15, wherein the glycine replacement mutation is G134A.

17. The mutant according to any one of claims 1 to 16 wherein the mutant comprises a cleavage site mutation.

18. The mutant according to 17, wherein the cleavage site mutation is F113G and F114S.

19. The mutant according to claim 1, wherein the mutant comprises the mutations selected from the group consisting of:
(1) Q92C-G134C;
(2) A466L;
(3) A466V;
(4) S473V;
(5) S473L;
(6) A480L;
(7) A466L and S473A;
(8) A466L and S473L;
(9) T198A;
(10) G134A;
(11) A128P;
(12) F113G, F114S, Q92C-G134C, A466L, S473L and A480L;
(13) Q92C-G134C, A466L, S473L and A480L;
(14) Q92C-G134C, A466L and S473L;
(15) F113G, F114S, Q92C-G134C, A466V, S473V and A480V;
(16) Q92C-G134C, A466V, S473V and A480V;
(17) Q92C-G134C, A466V and S473V;
(18) F113G, F114S, A466L, S473L, A480L and G134A;
(19) A466L, S473L, A480L and G134A;
(20) A466L, S473L and G134A;
(21) F113G, F114S, A466L, S473L, A480L, Q92A and G134A;
(22) F113G, F114S, A466L, S473L and G134A;
(23) A466L, S473L, A480L, Q92A, G134A;
(24) A466L, S473L, Q92A, G134A;
(25) F113G, F114S, Q92L, G134A;
(26) A466L, S473L, A480L, Q92L and G134A;
(27) A466L, S473L, Q92L and G134A;
(28) F113G, F114S, A466L, S473L, A480L, Q92A and G134L;
(29) A466L, S473L, A480L, Q92A and G134L;
(30) F113G, F114S, Q92C-G134C, A4661, S4731 and A480L;
(31) F113G, F114S, Q92C-G134C, A4661 and, S4731; and,
(32) A4661, S4731, A480L, Q92L and G134A.

20. The mutant according to claim 1, wherein the mutant comprises the mutations A466L, S473L, A480L and G134A.

21. The mutant according to claim 1, wherein the mutant comprises the mutations F113G, F114S, A466L, S473L and G134A.

22. The mutant according to claim 1, wherein the mutant comprises the mutations F113G, F114S, A466L, S473L, A480L and G134A.

23. The mutant according to claim 1, wherein the mutant comprises the mutations F113G, F114S, Q92C, G134C, A466L, S473L and A480L.

24. The mutant according to claim 1 wherein the mutant comprises a leucine at position 466, 473 and 480 (466L, 473L and 480L) and an alanine at position 134 (134A) and wherein the mutant comprises a F1 polypeptide and a F2 polypeptide selected from the group consisting of:
(1) a F2 polypeptide comprising the amino acid sequence of SEQ ID NO:255 and a F1 polypeptide comprising the amino acid sequence of SEQ ID NO:254;
(2) a F2 polypeptide comprising an amino acid sequence that is at least 97%, 98% or 99% identical to the amino acid sequence of SEQ ID NO:255 and a F1 polypeptide comprising an amino acid sequence that is at least 97%, 98%, or 99% identical to the amino acid sequence of SEQ ID NO:254.

25. The mutant according to claim 1 wherein the mutant comprises a glycine (G) at position 113 (113G), a serine at position 114 (114S), a leucine at position 466 and 473 (466L and 473L) and an alanine at position 134 (134A) and wherein the mutant comprises a F1 polypeptide and a F2 polypeptide selected from the group consisting of:
(1) a F2 polypeptide comprising the amino acid sequence of SEQ ID NO:291 and a F1 polypeptide comprising the amino acid sequence of SEQ ID NO:290;
(2) a F2 polypeptide comprising an amino acid sequence that is at least 97%, 98% or 99% identical to the amino acid sequence of SEQ ID NO:291 and a F1 polypeptide comprising an amino acid sequence that is at least 97%, 98%, or 99% identical to the amino acid sequence of SEQ ID NO:290.

26. The mutant according to claim 1 wherein the mutant comprises a glycine (G) at position 113 (113G), a serine at position 114 (114S), a leucine at position 466, 473 and 480 (466L, 473L and 480L) and an alanine at position 134 (134A) and wherein the mutant comprises a F1 polypeptide and a F2 polypeptide selected from the group consisting of:
(1) a F2 polypeptide comprising the amino acid sequence of SEQ ID NO:277 and a F1 polypeptide comprising the amino acid sequence of SEQ ID NO:276;
(2) a F2 polypeptide comprising an amino acid sequence that is at least 97%, 98% or 99% identical to the amino acid sequence of SEQ ID NO:277 and a F1 polypeptide comprising an amino acid sequence that is at least 97%, 98%, or 99% identical to the amino acid sequence of SEQ ID NO:276.

27. The mutant according to claim 1 wherein the mutant comprises a glycine (G) at position 113 (113G), a serine at position 114 (114S), a leucine at position 466, 473 and 480 (466L, 473L and 480L) and a cysteine at position 92 and 134 (92C and 134C) wherein the mutant comprises a F1 polypeptide and a F2 polypeptide selected from the group consisting of:
(1) a F2 polypeptide comprising the amino acid sequence of SEQ ID NO:273 and a F1 polypeptide comprising the amino acid sequence of SEQ ID NO:272;
(2) a F2 polypeptide comprising an amino acid sequence that is at least 97%, 98% or 99% identical to the amino acid sequence of SEQ ID NO:273 and a F1 polypeptide comprising an amino acid sequence that is at least 97%, 98%, or 99% identical to the amino acid sequence of SEQ ID NO:272.

28. The mutant according to any one of claims 1 to 27, wherein the F1 polypeptide lacks the cytoplasmic domain and the transmembrane domain.

29. The mutant according to any one of claims 1 to 27, wherein the F1 polypeptide lacks the cytoplasmic domain and the a portion of the transmembrane domain.

30. The mutant according to any one of claims 1 to 27 wherein the F1 polypeptide comprises or consists of amino acid residues 113 to 477 or comprises or consists of amino acid residues 113 to 480.

31. The mutant according to any one of claims 1 to 27, wherein the F1 polypeptide comprises the ectodomain, the transmembrane domain and the cytoplasmic domain.

32. The mutant according to any one of claims 1 to 31, wherein the mutant is linked to a trimerization domain.

33. The mutant according to claim 32, wherein the trimerization domain is a phage T4 fibritin foldon.

34. The mutant according to claim 33, wherein the trimerization domain is a phage T4 fibritin foldon of SEQ ID NO.7.

35. The mutant according to any one of claims 32 to 34, wherein the trimerization domain is linked to the C-terminus of the F1 polypeptide.

36. The mutant according to any one of claims 32 to 35, wherein the trimerization domain is linked to the C-terminus of the F1 polypeptide via a linker.

37. The mutant according to claim 36, wherein the linker is GGGS.

38. The mutant according to any one of claims 1 to 37, wherein the mutant is in the form of a trimer.

39. The mutant according to any one of claims 1 to 38, wherein the mutant is in the prefusion conformation.

40. The mutant according to any one of claims 1 to 39, wherein the mutant is in the prefusion conformation and specifically binds to an antibody (such as PIV1-8 mAb) specific for the PIV1 F ectodomain in the prefusion, but not postfusion, conformation.

41. The mutant of any one of claims 1 to 40 wherein the wild-type PIV1 is SEQ ID NO:206.

42. The mutant of any one of claim 1 to 41 wherein the amino acid positions correspond to the amino acid sequence of a reference of SEQ ID NO:206.

43. A nucleic acid comprising at least one coding sequence encoding at least one mutant of a wild-type PIV 1 F protein according to any one of claims 1 to 42, or an immunogenic fragment or immunogenic variant thereof, wherein the nucleic acid comprises at least one heterologous untranslated region (UTR).

44. A nucleic acid according to claim 43, wherein the at least one heterologous untranslated region is selected from at least one heterologous 5′-UTR and/or at least one heterologous 3′-UTR.

45. A nucleic acid according to claim 43 or 44, wherein the at least one heterologous 3′-UTR comprises or consists of a nucleic acid sequence having at least, at most, exactly, or between any two of 99%, 98%, 97%, 96%, 95%, 90%, 85%, or 80% identity to CΨCGAGCΨGGΨACΨGCAΨGCA CGCAAΨGCΨAGCΨGCCCCΨΨΨCCCGΨCCΨG GGΨACCCCGA GΨCΨCCCCCG ACCΨCGGGΨC CCAGGΨAΨGC ΨCCCACCΨCC ACCYGCCCCA CYCACCACCY CΨGCΨAGΨΨC CAGACACCYC CCAAGCACGC AGCAAΨGCAG CΨCAAAACGC ΨΨAGCCΨAGC CACACCCCCA CGGGAAACAG CAGΨGAΨΨAA CCΨΨΨAGCAA ΨAAACGAAAG ΨΨΨAACΨAAG CΨAΨACΨAAC CCCAGGGΨΨG GΨCAAΨΨΨCG ΨGCCAGCCAC ACCCΨGGAGC YAGC.

46. A nucleic acid according to any one of claims 43 to 45, wherein the at least one heterologous 5′-UTR comprises or consists of a nucleic acid sequence having at least, at most, exactly, or between any two of 99%, 98%, 97%, 96%, 95%, 90%, 85%, or 80% identity to GAAΨAAAC ΨAGΨAΨΨCΨΨCYGGYCCCCA CAGACYCAGA GAGAACCCGC CACC.

47. A nucleic acid according to any one of claims 43 to 46, wherein the nucleic acid comprises at least one poly(A) sequence, preferably comprising 30 to 200 adenosine nucleotides and/or at least one poly(C) sequence, preferably comprising 10 to 40 cytosine nucleotides.

48. A nucleic acid according to any one of claims 43 to 47, wherein the nucleic acid is a DNA or an RNA.

49. A nucleic acid according to claim 48, wherein the nucleic acid is a coding RNA.

50. A nucleic acid according to claim 49, wherein the coding RNA is an mRNA, a self-replicating RNA, a circular RNA, or a replicon RNA.

51. A nucleic acid according to claim 50, wherein the nucleic acid, preferably the coding RNA, is an mRNA.

52. A nucleic acid according to claim 51, wherein the mRNA is not a replicon RNA or a self-replicating RNA.

53. A nucleic acid according to any one of claims 50 to 52, wherein the mRNA comprises at least one poly(A) sequence comprising 30 to 200 adenosine nucleotides and the 3′ terminal nucleotide is an adenosine.

54. A nucleic acid according to any one of claims 48 to 53, wherein the RNA, preferably the coding RNA, comprises a 5′-cap structure, preferably m7G, capO, cap1, cap2, a modified capO or a modified cap1 structure, preferably a 5′-cap1 structure.

55. A nucleic acid according to any one of claims 48 to 54, wherein the RNA is codon-optimized.

56. A nucleic acid according to any one of claims 48 to 55, wherein the RNA comprises a chemically modified nucleotide.

57. A nucleic acid according to any one of claims 49 to 56, wherein the RNA comprises 1-methylpseudouridine substitution.

58. A nucleic acid according to claim 57, wherein all the uridines of the RNA are replaced by 1-methylpseudouridine.

59. A nucleic acid according to any one of claims 49 to 58, wherein the RNA is a purified RNA, preferably an RNA that has been purified by RP-HPLC and/or TFF.

60. A nucleic according to any one of claims 48 to 59 wherein the RNA comprises the nucleic acid sequence of any of SEQ ID NO:403, SEQ ID NO:405, SEQ ID NO:407 and SEQ ID NO:409.

61. A composition comprising at least one nucleic acid according to any one of claims 43 to 60.

62. A composition comprising at least one nucleic acid according to any one of claims 43 to 60, wherein the composition comprises at least one pharmaceutically acceptable carrier.

63. A composition comprising at least one nucleic acid according to any one of claims 43 to 60, wherein the composition is a multivalent composition comprising a plurality or at least more than one of the nucleic acid according to any one of claims 43 to 60.

64. A composition comprising at least one nucleic acid according to any one of claims 43 to 60, wherein the composition comprises RNA with an RNA integrity of 70% or more.

65. A composition comprising at least one nucleic acid according any one of claims 43 to 60, wherein the composition comprises RNA with a capping degree of 70% or more, preferably wherein at least 70%, 80%, or 90% of the mRNA species comprise a Cap1 structure.

66. A composition comprising at least one nucleic acid according to any one of claims 43 to 60, wherein the at least one nucleic acid is complexed or associated with or at least partially complexed or partially associated with one or more cationic or polycationic compound, preferably cationic or polycationic polymer, cationic or polycationic polysaccharide, cationic or polycationic lipid, cationic or polycationic protein, cationic or polycationic peptide, or any combinations thereof.

67. A composition comprising at least one nucleic acid according to any one of claims 43 to 60, wherein the at least one nucleic acid is complexed or associated with one or more lipids or lipid-based carriers, thereby forming liposomes, lipid nanoparticles (LNP), lipoplexes, and/or nanoliposomes, preferably encapsulating the at least one nucleic acid.

68. A composition comprising at least one nucleic acid according to any one of claims 43 to 60, wherein the at least one nucleic acid is complexed with one or more lipids thereby forming lipid nanoparticles.

69. A composition according to any one of claim 67 or 68, wherein the LNP comprises a cationic lipid according to formula III-3:

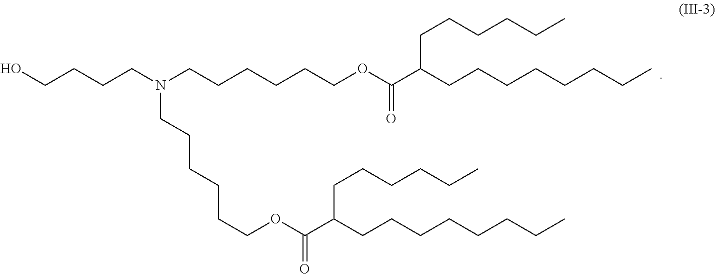 [SMILES: CCCCCCCCC(CCCCCC)C(=O)OCCCCCCN(CCCCO)CCCCCCOC(=O)C(CCCCCC)CCCCCCCC]

70. A composition according to any one of claims 67 to 69, wherein the LNP comprises a PEG lipid of formula (IVa):

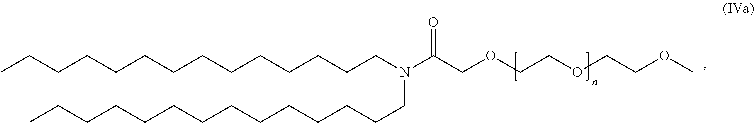

71. A composition according to claim 70, wherein n has a mean value ranging from 30 to 60, preferably wherein n has a mean value of about 45, 46, 47, 48, 49, 50, 51, 52, 53, 54, most preferably wherein n has a mean value of 49 or 45.

72. A composition according to any one of claims 67 to 71, wherein the LNP comprises a PEG lipid of formula (IVa): 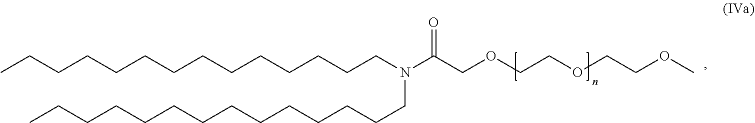
wherein n is an integer selected such that the average molecular weight of the PEG lipid is about 2500 g/mol.

73. A composition according to any one of claims 67 to 72, wherein the LNP comprises one or more neutral lipids and/or one or more steroid or steroid analogues.

74. A composition according to claim 73, wherein the neutral lipid is 1,2-distearoyl-sn-glycero-3-phosphocholine (DSPC), preferably wherein the molar ratio of the cationic lipid to DSPC is in the range from about 2:1 to about 8:1.

75. A composition according to claim 73 or 74, wherein the steroid is cholesterol, preferably wherein the molar ratio of the cationic lipid to cholesterol is in the range from about 2:1 to about 1:1.

76. A composition according to any one of claims 67 to 75, wherein the LNP comprises (i) at least one cationic lipid, preferably a lipid of formula (III), more preferably lipid III-3; (ii) at least one neutral lipid, preferably 1,2-distearoyl-sn-glycero-3-phosphocholine (DSPC); (iii) at least one steroid or steroid analogue, preferably cholesterol; and (iv) at least one polymer conjugated lipid, preferably a PEG-lipid derived from formula (IVa, with n=49), wherein (i) to (iv) are in a molar ratio of about 20-60% cationic lipid, 5-25% neutral lipid, 25-55% sterol, and 0.5-15% PEG-lipid.

77. A composition according to any one of claims 67 to 76, wherein the LNP comprises (i) at least one cationic lipid, preferably a lipid of formula (III), more preferably lipid 111-3; (ii) at least one neutral lipid, preferably 1,2-distearoyl-sn-glycero-3-phosphocholine (DSPC); (iii) at least one steroid or steroid analogue, preferably cholesterol; and (iv) at least one polymer conjugated lipid, preferably a PEG-lipid derived from formula (IVa, with n=45), wherein (i) to (iv) are in a molar ratio of about 20-60% cationic lipid, 5-25% neutral lipid, 25-55% sterol, and 0.5-15% PEG-lipid.

78. A composition according to claim 76 or 77, wherein (i) to (iv) are in a molar ratio of about 50:10:38.5:1.5, preferably 47.5:10:40.8:1.7 or more preferably 47.4:10:40.9:1.7.

79. A composition according to any one of claims 67 to 78, wherein the nucleic acid is RNA and the composition comprises less than about 20% free (non complexed or non-encapsulated) RNA, preferably less than about 15% free RNA, more preferably less than about 10% free RNA.

80. A composition according to any one of claims 67 to 79, wherein the wt/wt ratio of lipid to nucleic acid is from about 10:1 to about 60:1, preferably from about 20:1 to about 30:1, for example about 25:1.

81. A composition according to any one of claims 67 to 80, wherein the n/p ratio of the LNPs encapsulating the nucleic acid is in a range from about 1 to about 10, preferably in a range from about 5 to about 7, more preferably about 6.

82. A composition according to any one of claims 67 to 81, wherein the composition has a polydispersity index (PDI) value of less than about 0.4, preferably of less than about 0.3, more preferably of less than about 0.2, most preferably of less than about 0.1.

83. A composition comprise according to any one of claims 67 to 82, wherein the LNPs have a Z-average size in a range of about 60 nm to about 120 nm, preferably less than about 120 nm, more preferably less than about 100 nm, most preferably less than about 80 nm.

84. A composition according to any one of claims 67 to 83, wherein the LNPs comprise less than about 10%, 9%, 8%, 7%, 6%, 5%, 4%, 3%, 2%, 1% LNPs that have a particle size exceeding about 500 nm.

85. A composition according to any one of claims 67 to 84, wherein the LNPs comprise less than about 10%, 9%, 8%, 7%, 6%, 5%, 4%, 3%, 2%, 1% LNPs that have a particle size smaller than about 20 nm.

86. A composition according to any one of claims 67 to 85, wherein the LNP comprises (i) at least one cationic lipid; (ii) at least one neutral lipid; (iii) at least one steroid or steroid analogue;
and (iv) at least one PEG-lipid, wherein (i) to (iv) are in a molar ratio of about 20-60% cationic lipid, 5-25% neutral lipid, 25-55% sterol, and 0.5-15% PEG-lipid.

87. A composition according to any one of claims 67 to 86, wherein the LNP comprises (i) at least one cationic lipid according to formula III-3; (ii) DSPC; (iii) cholesterol; and (iv) a PEG-lipid, according to formula IVa, wherein (i) to (iv) are in a molar ratio of about 20-60% cationic lipid, 5-25% neutral lipid, 25-55% sterol, and 0.5-15% PEG-lipid.

88. A composition according to any one of claims 67 to 87, wherein the composition is a lyophilized composition.

89. An immunogenic composition comprising a mutant according to any one of claims 1 to 42 or a nucleic acid according to any one of claims 43 to 60 or a composition according to any one of claims 61 to 88.

90. An immunogenic composition according to claim 89, further comprising a hMPV A antigen selected from the group consisting of a mutant of a wild-type hMPV A F protein and a nucleic acid encoding a mutant of a wild-type hMPV A F protein.

91. An immunogenic composition according to claim 90, wherein the hMPV A antigen is a mutant of a wild-type hMPV A F protein.

92. An immunogenic composition according to claim 91, wherein the hMPV A antigen is a mutant of a wild-type hMPV A F protein from the present disclosure, preferably from any of E1 to E72 of section B of the present disclosure.

93. An immunogenic composition according to claim 90, wherein the hMPV A antigen comprises a nucleic acid encoding a mutant of a wild-type hMPV A F protein.

94. An immunogenic composition according to claim 93, wherein the hMPV A antigen comprises a nucleic acid encoding a mutant of a wild-type hMPV A F protein from the present disclosure, preferably from any of E73 to E89 of section B of the present disclosure.

95. An immunogenic composition according to any one of claims 89 to 94, further comprising a hMPV B antigen selected from the group consisting of a mutant of a wild-type hMPV B F protein and a nucleic acid encoding a mutant of a wild-type hMPV B F protein.

96. An immunogenic composition according to claim 95, wherein the hMPV B antigen is a mutant of a wild-type hMPV B F protein.

97. An immunogenic composition according to claim 96, wherein the hMPV B antigen is a mutant of a wild-type hMPV B F protein from the present disclosure, preferably from any of E1 to E72 of section B of the present disclosure.

98. An immunogenic composition according to claim 95, wherein the hMPV B antigen comprises is a nucleic acid encoding a mutant of a wild-type hMPV B F protein.

99. An immunogenic composition according to claim 97, wherein the hMPV B antigen comprises a nucleic acid encoding a mutant of a wild-type hMPV B F protein from the present disclosure, preferably from any of E73 to E89 of section B of the present disclosure.

100. An immunogenic composition according to any one of claims 89 to 99, further comprising PIV3 antigen selected from the group consisting of a mutant of a wild-type PIV3 F protein and a nucleic acid encoding a mutant of a wild-type PIV3 F protein.

101. An immunogenic composition according to claim 100, wherein the PIV3 antigen is a mutant of a wild-type PIV3 F protein.

102. An immunogenic composition according to claim 101, wherein the PIV3 antigen is a mutant of a wild-type PIV3 F protein from the present disclosure, preferably from any of E1 to E52 of section D of the present disclosure.

103. An immunogenic composition according to claim 101, wherein the PIV3 antigen is a mutant of a wild-type PIV3 F protein as disclosed in WO2018081289 or WO2022207839.

104. An immunogenic composition according to claim 100, wherein the PIV3 antigen comprises a nucleic acid encoding a mutant of a wild-type PIV3 F protein.

105. An immunogenic composition according to claim 104, wherein the PIV3 antigen comprises a nucleic acid encoding a mutant of a wild-type PIV3 F protein from the present disclosure, preferably from any of E53 to E69 of section D of the present disclosure.

106. An immunogenic composition according to claim 104, wherein the PIV3 antigen comprises a nucleic acid encoding a mutant of a wild-type PIV3 F protein as disclosed in WO2018081289 or WO2022207839.

107. An immunogenic composition according to any one of claims 89 to 106, further comprising an RSV antigen selected from the group consisting of a mutant of a wild-type RSV F protein of subtype A and a nucleic acid encoding a mutant of a wild-type RSV F protein of subtype A.

108. An immunogenic composition according to claim 107, wherein the RSV antigen is a mutant of a wild-type RSV F protein of subtype A.

109. An immunogenic composition according to claim 107, wherein the RSV antigen is a nucleic acid encoding a mutant of a wild-type RSV F protein of subtype A.

110. An immunogenic composition according to claim 108, wherein the mutant of a wild-type RSV F protein of subtype A is disclosed in one of WO2009/079796, WO2010/149745, WO2011/008974, WO2014/160463, WO2014/174018, WO2014/202570, WO2015/013551, WO2015/177312, WO2017/005848, WO2017/174564, WO2017/005844, WO2017/109629, WO2022/002894 and WO2018/109220.

111. An immunogenic composition according to any one of claim 89 to claim 110, further comprising an RSV antigen selected from the group consisting of a mutant of a wild-type RSV F protein of subtype B and a nucleic acid encoding a mutant of a wild-type RSV F protein of subtype B.

112. An immunogenic composition according to claim 111, wherein the RSV antigen is a mutant of a wild-type RSV F protein of subtype B.

113. An immunogenic composition according to claim 111, wherein the RSV antigen is a nucleic acid encoding a mutant of a wild-type RSV F protein of subtype B.

114. An immunogenic composition according to claim 111, wherein the mutant of a wild-type RSV F protein of subtype B is disclosed in one of WO2009/079796, WO2010/149745, WO2011/008974, WO2014/160463, WO2014/174018, WO2014/202570, WO2015/013551, WO2015/177312, WO2017/005848, WO2017/174564, WO2017/005844, WO2017/109629, WO2022/002894 and WO2018/109220.

115. A mutant of a wild-type hMPV F protein as defined in any of embodiment E1 to E73 disclosed in above section B.

116. A nucleic acid comprising at least one coding sequence encoding at least one mutant of a wild-type hMPV F protein, said nucleic acid being as defined in any of embodiments E74 to E89 disclosed in above section B.

117. A composition as defined in any of embodiments E90 to E117 disclosed in above section B.

118. An immunogenic composition as defined in any of embodiments E118 to E139 disclosed in above section B.

119. A mutant of a wild-type PIV3 F protein as defined in any of embodiment E1 to E52 disclosed in above section D.

120. The mutant according to claim 119 wherein the mutant comprises the mutations E209C and L234C.

121. The mutant according to claim 120 wherein the mutant comprises the mutations E209C, L234C, S160C and V170C.

122. The mutant according to claim 119 wherein the mutant comprises the mutations selected from the group consisting of
(1) G230A, S470A and S477A;
(2) S160C, V170C, G230A and A463L;
(3) S160C, V170C, S470A and S477A;
(4) S160C, V170C, G230A, S470A and S477A;
(5) S160C, V170C, G230A, A463L, S470A and S477 Å (6) S160C, V170C, E209C, L234C, A463L and S470L;
(7) S160C, V170C, E209C, L234C, A463L and 1474F;
(8) S160C, V170C, E209C, L234C, A463L, S470L, F110G, F111S;
(9) S160C, V170C, A463L and S470L, and,
(10) E209C, L234C.

123. The mutant according to claim 119 wherein,
(a) the mutant comprises a cysteine at position 160 (160C), 170 (170C), 209 (209C) and 234 (234C) and a leucine at position 463 (463L) and 470 (470L), wherein the mutant comprises a F1 polypeptide and a F2 polypeptide selected from the group consisting of:
(1) a F2 polypeptide comprising the amino acid sequence of SEQ ID NO:438 and a F1 polypeptide comprising the amino acid sequence of SEQ ID NO:437;
(2) a F2 polypeptide comprising an amino acid sequence that is at least 97%, 98% or 99% identical to the amino acid sequence of SEQ ID NO:438 and a F1 polypeptide comprising an amino acid sequence that is at least 97%, 98%, or 99% identical to the amino acid sequence of SEQ ID NO:437; or,
(b) the mutant comprises a cysteine at position 160 (160C), 170 (170C), 209 (209C) and 234 (234C) and a leucine at position 463 (463L) and a phenylalanine at position 474 (474F), wherein the mutant comprises a F1 polypeptide and a F2 polypeptide selected from the group consisting of:
(1) a F2 polypeptide comprising the amino acid sequence of SEQ ID NO:440 and a F1 polypeptide comprising the amino acid sequence of SEQ ID NO:439;
(2) a F2 polypeptide comprising an amino acid sequence that is at least 97%, 98% or 99% identical to the amino acid sequence of SEQ ID NO:440 and a F1 polypeptide comprising an amino acid sequence that is at least 97%, 98%, or 99% identical to the amino acid sequence of SEQ ID NO:439; or,
(c) the mutant comprises a cysteine at position 160 (160C), 170 (170C), 209 (209C) and 234 (234C), a leucine at position 463 (463L) and 470 (470L), a glycine at position 110 (110G) and a serine at position 111 (111S) wherein the mutant comprises a F1 polypeptide and a F2 polypeptide selected from the group consisting of:
(1) a F2 polypeptide comprising the amino acid sequence of SEQ ID NO:482 and a F1 polypeptide comprising the amino acid sequence of SEQ ID NO:481;
(2) a F2 polypeptide comprising an amino acid sequence that is at least 97%, 98% or 99% identical to the amino acid sequence of SEQ ID NO:482 and a F1 polypeptide comprising an amino acid sequence that is at least 97%, 98%, or 99% identical to the amino acid sequence of SEQ ID NO:481; or,
(d) the mutant comprises a cysteine at position 160 (160C) and 170 (170C) and a leucine at position 463 (463L) and 470 (470L), wherein the mutant comprises a F1 polypeptide and a F2 polypeptide selected from the group consisting of:
(1) a F2 polypeptide comprising the amino acid sequence of SEQ ID NO:494 and a F1 polypeptide comprising the amino acid sequence of SEQ ID NO:493;
(2) a F2 polypeptide comprising an amino acid sequence that is at least 97%, 98% or 99% identical to the amino acid sequence of SEQ ID NO:494 and a F1 polypeptide comprising an amino acid sequence that is at least 97%, 98%, or 99% identical to the amino acid sequence of SEQ ID NO:493.

124. A nucleic acid comprising at least one coding sequence encoding at least one mutant of a wild-type PIV3 F protein, said nucleic acid being as defined in any of embodiments E53 to E69 disclosed in above section D.

125. A nucleic acid according to claim 124 wherein the nucleic acid is an mRNA and comprises the nucleic acid sequence of any of SEQ ID NO:411, SEQ ID NO:413, SEQ ID NO:415, SEQ ID NO:417 and SEQ ID NO:419, SEQ ID NO:504, SEQ ID NO:506, SEQ ID NO:508 and SEQ ID NO:510, SEQ ID NO: 295, SEQ ID NO: 297 and SEQ ID NO: 299.

126. A composition as defined in any of embodiments E70 to E97 disclosed in above section D.

127. An immunogenic composition as defined in any of embodiments E98 to E122 disclosed in above section D.

128. An isolated antibody that binds to human metapneumovirus (hMPV), comprising a heavy chain variable region (hMPV-VH) and a light chain variable region (hMPV-VL), comprising the CDR-H1, CDR-H2, and CDR-H3 sequences of SEQ ID NO: 360, and the CDR-L1, CDR-L2, and CDR-L3 sequences of SEQ ID NO: 361.

129. An isolated antibody that binds to hMPV, comprising a heavy chain variable region (hMPV-VH) and a light chain variable region (hMPV-VL), comprising a CDR-H1 sequence according to SEQ ID NO: 523 or 524; a CDR-H2 sequence according to SEQ ID NO: 525 or 526; a CDR-H3 sequence according to SEQ ID NO: 527 or 528 and comprising a CDR-L1 sequence according to SEQ ID NO: 529; a CDR-L2 sequence according to SEQ ID NO: 530, and a CDR-L3 sequence according to SEQ ID NO: 531.

130. The antibody of either claim 128 or 129, comprising a hMPV-VH sequence of SEQ ID NO: 360, and comprising a hMPV-VL sequence of SEQ ID NO: 361.

131. An isolated antibody that binds to parainfluenza virus type 1 (PIV1), comprising a heavy chain variable region (PIV1-VH) and a light chain variable region (hMPV-VL), comprising the CDR-H1, CDR-H2, and CDR-H3 sequences of SEQ ID NO: 362, and the CDR-L1, CDR-L2, and CDR-L3 sequences of SEQ ID NO: 363.

132. An isolated antibody that binds to PIV1, comprising a heavy chain variable region (PIV1-VH) and a light chain variable region (PIV1-VL), comprising a CDR-H1 sequence according to SEQ ID NO: 534 or 535; a CDR-H2 sequence according to SEQ ID NO: 536 or 537; a CDR-H3 sequence according to SEQ ID NO: 538 or 539 and comprising a CDR-L1 sequence according to SEQ ID NO: 540; a CDR-L2 sequence according to SEQ ID NO: 541, and a CDR-L3 sequence according to SEQ ID NO: 542.

133. The antibody of either claim 132 or 133, comprising a PIV1-VH sequence of SEQ ID NO: 362, and comprising a PIV1-VL sequence of SEQ ID NO: 363.
